(19) United States
(12) Patent Application Publication
(10) Pub. No.: US 2016/0120863 A1
(43) Pub. Date: May 5, 2016

(54) SLC2A TRANSPORTER INHIBITORS

(71) Applicant: IOMET PHARMA LTD., Edinburgh (GB)

(72) Inventors: Phillip Martin Cowley, Edinburgh (GB); Alan Wise, Edinburgh (GB); Thomas James Brown, Edinburgh (GB); Matthew Isherwood, Galen (SG); Anjan Chakrabarti, Galen (SG)

(21) Appl. No.: 14/893,513

(22) PCT Filed: May 22, 2014
(86) PCT No.: PCT/EP2014/060586

§ 371 Date: Nov. 23, 2015

(30) Foreign Application Priority Data
May 24, 2013 (GB) 1309405.7
Feb. 11, 2014 (GB) 1402341.0

(51) Int. Cl. A61K 31/496; G01N 33/50; A61K 45/06; C07D 471/04; A61K 31/5377

(52) U.S. Cl. CPC A61K 31/496; C07D 471/04; A61K 31/5377; A61K 45/06; G01N 33/502; G01N 2500/10; G01N 2500/04; G01N 2333/705

(57) ABSTRACT

Provided is a SLC2A class I transporter inhibitor compound for use in medicine, which compound comprises the following formula:

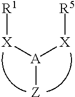

wherein A and Z may be the same or different and are each independently selected from C, N, O and S; each X may be the same or different and is independently selected from C, N, O and S; R¹ and R⁵ may be present or absent and may be the same or different and are each selected from H and a substituted or unsubstituted organic group; Z completes a ring with each X, each ring comprising from 3 to 8 ring atoms including the X, A, and Z, each ring atom being independently selected from C, N, O and S, and each ring atom being unsubstituted or independently substituted with H or a substituted or unsubstituted organic group; and wherein the bonds between all of the atoms in the rings including the X, A, and Z may independently be single bonds or double bonds, provided that when X or a ring atom is O or S the bonds to X are single bonds.

The present invention relates to SLC2A class I transporter inhibitors (such as GLUT1 and GLUT2 inhibitors), and in particular SLC2A class I transporter inhibitors for use in medicine. The inhibitors of the invention may be used in pharmaceutical compositions, and in particular pharmaceutical compositions for treating a cancer, an inflammatory condition, a metabolic condition, a neurological condition, a proliferative disorder, and/or an autoimmune condition. The invention also relates to methods of screening for such inhibitors, methods of manufacture of such inhibitors, and methods of treatment using such inhibitors.

Glucose is an essential substrate for metabolism in most cells. It provides energy in the form of ATP through glycolysis and the citric acid cycle, and reducing power in the form of NADPH through the pentose phosphate shunt. It is also used in the synthesis of glycerol for triglyceride production and provides intermediates for synthesis of nonessential amino acids. Because glucose is a polar molecule, transport through biological membranes requires specific transport proteins. Hence, the plasma membranes of virtually all mammalian cells possess one or more transport systems to allow glucose movement either into or out of the cells.

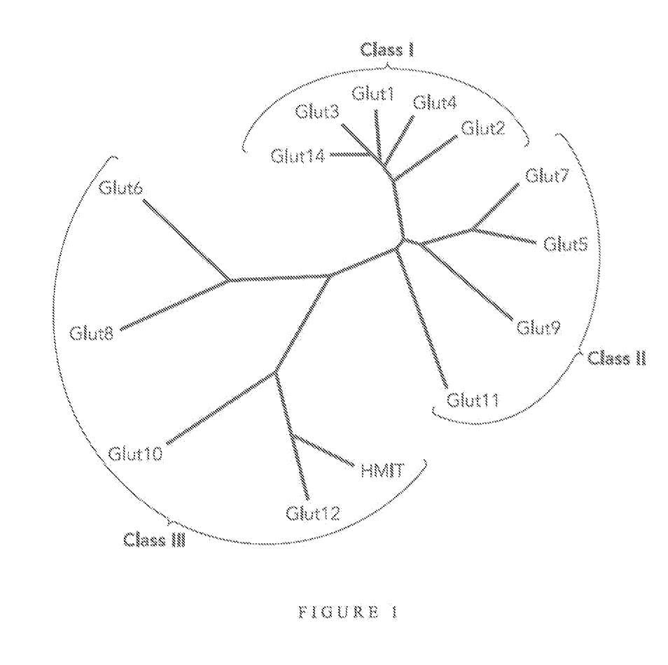

Mammalian cells take up glucose from extracellular fluid into the cell through two families of structurally related glucose transporters. The facilitative glucose transporter family (solute carriers SLC2A, protein symbol GLUT) mediates a bidirectional and energy-independent process of glucose transport in most tissues and cells, while the Na+/glucose co-transporter family (solute carriers SLC5A, protein symbol SGLT) mediates an active, Na+-linked transport process against an electrochemical gradient (see Table 1). The GLUT family consists of fourteen members (GLUT1 to 12, 14 and HMIT). Phylogenetically, the members of the GLUT family are split into three classes (class I, class II and class III) based on structural and protein sequence similarities (see FIG. 1). The class I SLC2A GLUT family comprises five members: GLUT1 to 4 and GLUT14. The facilitative transport mediated by the GLUT family is inhibitable by cytochalasin-B, or phloretin. In the epithelial cell brush border of the small intestine and the kidney proximal convoluted tubules only, glucose is absorbed or reabsorbed against its electrochemical gradient by the SGLT-mediated secondary active transport mechanism using the sodium concentration gradient established by Na+/K+/ATP pumps. Up to present, at least six members of the SGLT family have been cloned (SGLT1-6) but only SGLT-1 and SGLT-2 have been well characterized.

TABLE 1
Summary of the Properties of Facilitative Glucose Transporter and Na+/Glucose co-Transporter Family Members: (from F.
Q. Zhao and A. F. Keating, Functional properties and genomics of glucose transporters, Curr Genomics, 2007, 113-128)

| | | | | |
|---|---|---|---|---|
| | Major | Kₘ² | | |
| Protein | isoform (aa)¹ | (mM) | Major sites of expression | Proposed function |

Facilitative glucose transporters (GLUT)

| | | | | |
|---|---|---|---|---|
| GLUT1 | 492 | 3-7 | Ubiquitous distribution in tissues and culture cells | Basal glucose uptake, transport across blood tissue barriers |
| GLUT2 | 524 | 17 | Liver, islets, kidney, small intestine | High-capacity low-affinity transport |
| GLUT3 | 496 | 1.4 | Brain and nerves cells | Neuronal transport |
| GLUT4 | 509 | 6.6 | Muscle, fat, heart | Insulin-regulated transport in muscle and fat |
| GLUT5 | 501 | | Intestine, kidney, testis | Transport of fructose |
| GLUT6 | 507 | ?³ | Spleen, leukocytes, brain | |
| GLUT7 | 524 | 0.3 | Small intestine, colon, testis | Transport of fructose |
| GLUT8 | 477 | 2 | Testis, blastocyst, brain, muscle, adipocytes | Fuel supply of mature spermatozoa; |
| | | | | Insulin-responsive transport in blastocyst |
| GLUT9 | 511/540 | ? | Liver, kidney | |
| GLUT10 | 541 | 0.3 | Liver, pancreas | |
| GLUT11 | 496 | ? | Heart, muscle | Muscle-specific; fructose transporter |
| GLUT12 | 617 | ? | Heart, prostate, mammary gland | |
| HMIT | 618/629 | ? | Brain | H⁻/myo-inositol co-transporter |

Na⁻/glucose cotransporter (SGLT)

| | | | | |
|---|---|---|---|---|
| SGLT1 | 664 | 0.2 | Kidney, intestine | Glucose reabsorption in intestine and kidney |
| SGLT2 | 672 | 10 | Kidney | Low affinity and high selectivity for glucose |
| SGLT3 | 660 | 2 | Small intestine, skeletal muscle | Glucose activated Na⁻ channel |

¹aa, amino acids.
²Net influx for 2-Deoxyglucose or glucose;
³? = unknown

Unlike normal cells, cancer cells have an unusual metabolic profile, exhibiting an addiction to glucose and a high rate of aerobic glycolysis to supply them with sufficient energy to meet their needs for rapid growth. This phenomenon, known as the ‘Warburg Effect’ is independent of the availability of oxygen and results in increased levels of lactate and low ATP production (O. Warburg, On respiratory impairment in cancer cells, Science, 1956, 269-270). This metabolic trait confers advantages to cancer cells by establishing a means of providing building blocks to support biomass synthesis for growth and proliferation, whilst still supplying the cells with sufficient energy production, even in the hypoxic environments often encountered in tumour tissue. The enhanced glucose uptake that accompanies the elevated rate of glycolysis in cancer cells is utilised to image cancers in the clinic using the glucose analogue 2-(18F)-fluoro-2-deoxy-D-glucose (FDG) by positron emission tomography (PET), and is the most commonly used tumour diagnostic tool (S. M. Larson and H. Schoder, Advances in positron emission tomography applications for urologic cancers, Curr Opin Urol, 2008, 65-70). However, this altered metabolism makes cancer cells more dependent on their primary energy source, glucose, than normal cells (G. Kroemer and J. Pouyssegur, Tumor cell metabolism: cancer's Achilles' heel, Cancer Cell, 2008, 472-482).

There is an established body of literature demonstrating up-regulation of class I SLC2A family sugar transporters in a variety of tumour types and modulation of their function either via gene knockdown or small molecule inhibition has shown significant effects on cancer cell growth in vitro and tumour growth in in vivo animal models of disease (reviewed in: M. B. Calvo, A. Figueroa, E. G. Pulido, R. G. Campelo and L. A. Aparicio, Potential role of sugar transporters in cancer and their relationship with anticancer therapy, Int J Endocrinol, 2010). Elevated levels of glucose uptake, one of the hallmarks of malignant cells, are induced by activated ras or src oncogenes which are key elements in the transduction of multiple signalling pathways. In this regard, it has been recently published that, in colorectal cancer cell lines, mutations in KRAS (v-Ki-ras2 Kirsten rat sarcoma viral oncogene homolog) or BRAF (vraf murine sarcoma viral oncogene homolog B1) genes, are able to trigger an overexpression of GLUT1 and an increase of glucose uptake (J. S. Flier, M. M. Mueckler, P. Usher and H. F. Lodish, Elevated levels of glucose transport and transporter messenger RNA are induced by ras or src oncogenes, Science, 1987, 1492-1495). The MYC oncogene also promotes up-regulation of both GLUT1 and GLUT3 and the concomitant elevation of glucose uptake (R. C. Osthus, H. Shim, S. Kim, Q. Li, R. Reddy, M. Mukherjee et al., Deregulation of glucose transporter 1 and glycolytic gene expression by c-Myc, J Biol Chem, 2000, 21797-21800).

The glucose transporter GLUT1 is the main protein responsible for glucose uptake into many cancer cells to enable them to fuel themselves and sustain their exaggerated growth. The expression of GLUT1 is induced under hypoxia and it is repressed by the tumour suppressor p53. Its expression level is correlated with invasiveness and metastasis potential of cancers indicating the importance of upregulation of glucose transport in cancer cell growth, and in the severity of cancer cell malignancy (M. B. Calvo, A. Figueroa, E. G. Pulido, R. G. Campelo and L. A. Aparicio, Potential role of sugar transporters in cancer and their relationship with anticancer therapy, Int J Endocrinol, 2010). Both FDG uptake and GLUT1 expression appear to be associated with increased tumour size. For example in several tumours such as NSCLC, colon cancer, bladder cancer, breast cancer and thyroid cancers, increased GLUT1 expression not only confers a malignant phenotype but also predicts for inferior overall survival (reviewed in M. B. Calvo, A. Figueroa, E. G. Pulido, R. G. Campelo and L. A. Aparicio, Potential role of sugar transporters in cancer and their relationship with anticancer therapy, Int J Endocrinol, 2010). In vitro Studies have also shown RNA-interference against GLUT1 expression reduces tumorigenicity (T. Amann, U. Maegdefrau, A. Hartmann, A. Agaimy, J. Marienhagen, T. S. Weiss et al., GLUT1 expression is increased in hepatocellular carcinoma and promotes tumorigenesis, Am J Pathol, 2009, 1544-1552). GLUT1 antibodies induce growth arrest and apoptosis in human cancer cell lines (S. Rastogi, S. Banerjee, S. Chellappan and G. R. Simon, GLUT1 antibodies induce growth arrest and apoptosis in human cancer cell lines, Cancer Lett, 2007, 244-251). Recently, small molecule inhibitors of SLC2A class I transporters have been demonstrated to selectively impair the growth of cancer cells in culture and in animal xenograft models (D. A. Chan, P. D. Sutphin, P. Nguyen, S. Turcotte, E. W. Lai, A. Banh et al., Targeting GLUT1 and the Warburg effect in renal cell carcinoma by chemical synthetic lethality, Sci Transl Med, 2011, 94ra70; Y. Liu, Y. Cao, W. Zhang, S. Bergmeier, Y. Qian, H. Akbar et al., A small-molecule inhibitor of glucose transporter 1 downregulates glycolysis, induces cell-cycle arrest, and inhibits cancer cell growth in vitro and in vivo, Mol Cancer Ther, 2012, 1672-1682)..

Tumour hypoxia has a well-defined role in driving tumour progression and metastasis as well as resistance to therapy. A key mediator of hypoxic stress is the heterodimeric transcription factor Hypoxia Inducible Factor, HIF. HIF is composed of an oxygen-labile subunit (HIF-α) and a constitutive subunit (HIF-3). In the presence of oxygen, hydroxylation on proline residues 564 and 402 by prolyl hydroxylases marks HIF-α for recognition and binding with the E3 ubiquitin ligase Von Hippel-Landau protein (pVHL), leading to the proteasomal degradation of HIF-α. Under hypoxia, activity of PHDs decrease, which prevents the recognition of HIF-α by pVHL (O. Iliopoulos, A. Kibel, S. Gray and W. G. Kaelin, Jr., Tumour suppression by the human von Hippel-Lindau gene product, Nat Med, 1995, 822-826). In cells lacking VHL, stabilized HIF-α binds HIF-β to activate transcription of genes including GLUT1 that are involved in diverse processes such as glycolysis, angiogenesis, tissue remodelling and epithelial permeability. Together, these processes act to promote tumour growth and survival in hypoxic conditions (N. P. Jones and A. Schulze, Targeting cancer metabolism-aiming at a tumour's sweet-spot, Drug Discov Today, 2012, 232-241).

Hence, the inventors have determined that an additional therapeutic application for GLUT1 inhibition would be in the treatment of patients defective in the von Hippel-Landau gene and diseases associated with such defects and in diseases mediated by HIF (e.g. angiomatosis, hemangioblastomas, pheochromocytomas and pancreatic cysts).

Renal cell carcinoma (RCC), the most common type of kidney cancer is a particularly intractable disease, often being resistant to both standard chemotherapies and radiation treatment. One key distinguishing feature in RCC is the loss of the VHL gene leading to exaggerated levels of HIF-α and its downstream targets (P. D. Sutphin, D. A. Chan, J. M. Li, S. Turcotte, A. J. Krieg and A. J. Giaccia, Targeting the loss of the von Hippel-Lindau tumor suppressor gene in renal cell carcinoma cells, Cancer Res, 2007, 5896-5905). Indeed, small molecule inhibition of GLUT1 activity has been found to be chemically synthetically lethal in VHL mutant RCC cell lines (D. A. Chan, P. D. Sutphin, P. Nguyen, S. Turcotte, E. W. Lai, A. Banh et al., Targeting GLUT1 and the Warburg effect in renal cell carcinoma by chemical synthetic lethality, Sci Transl Med, 2011, 94ra70). In the case of chemical synthetic lethality, the first mutation is essential to cancer development whilst the product of a second gene is inhibited by a small molecule resulting in cytotoxic cell death (L. H. Hartwell, P. Szankasi, C. J. Roberts, A. W. Murray and S. H. Friend, Integrating genetic approaches into the discovery of anticancer drugs, Science, 1997, 1064-1068). The inventors believe that this approach is attractive because it should not affect normal non-cancerous cells/tissue. Other cancers associated with VHL disruption include tumours of the eye, brain, spinal cord, kidney, pancreas and adrenal glands.

The highly related class I SLC2A transporter GLUT2 has also been shown to be highly expressed in a number of tumour samples (A. Godoy, V. Ulloa, F. Rodriguez, K. Reinicke, A. J. Yanez, L. Garcia Mde et al., Differential subcellular distribution of glucose transporters GLUT1-6 and GLUT9 in human cancer: ultrastructural localization of GLUT1 and GLUT5 in breast tumor tissues, J Cell Physiol, 2006, 614-627) and its expression has been positively associated with FDG uptake in hepatocellular carcinoma patient samples implying a role in tumour progression (B. Paudyal, N. Oriuchi, P. Paudyal, Y. Tsushima, Y. lida, T. Higuchi et al., Early diagnosis of recurrent hepatocellular carcinoma with 18F-FDG PET after radiofrequency ablation therapy, Oncol Rep, 2007, 1469-1473).

Up-regulation of another class I SLC2A family member GLUT3 has also been strongly linked to tumorigenesis. GLUT3 isoform, which is HIF and MYC-inducible, appears to be a predominant glucose transporter in highly malignant glial cells of human brain. Similarly targeting of GLUT3, which is involved in neovascularisation in glioblastoma has been suggested to prevent resistance to conventional therapy (B. Le Calve, M. Rynkowski, M. Le Mercier, C. Bruyere, C. Lonez, T. Gras et al., Long-term in vitro treatment of human glioblastoma cells with temozolomide increases resistance in vivo through up-regulation of GLUT transporter and aldo-keto reductase enzyme AKRIC expression, Neoplasia, 2010, 727-739). Oligonucleotide microarray analysis revealed that SLC2A3 (the gene encoding GLUT3) overexpression was correlated with tumour size, pathologic stage and recurrence in oral tongue carcinoma (C. L. Estilo, O. c. P, S. Talbot, N. D. Socci, D. L. Carlson, R. Ghossein et al., Oral tongue cancer gene expression profiling: Identification of novel potential prognosticators by oligonucleotide microarray analysis, BMC Cancer, 2009, 11). GLUT3 protein expression was also evaluated by immunohistochemistry as an indicator of poor prognosis outcome in non-small lung carcinoma, oral squamous cell carcinoma and laryngeal carcinoma (F. R. Ayala, R. M. Rocha, K. C. Carvalho, A. L. Carvalho, I. W. da Cunha, S. V. Lourenco et al., GLUT1 and GLUT3 as potential prognostic markers for Oral Squamous Cell Carcinoma, Molecules, 2010, 2374-2387; S. Baer, L. Casaubon, M. R. Schwartz, A. Marcogliese and M. Younes, GLUT3 expression in biopsy specimens of laryngeal carcinoma is associated with poor survival, Laryngoscope, 2002, 393-396; M. Younes, R. W. Brown, M. Stephenson, M. Gondo and P. T. Cagle, Overexpression of GLUT1 and GLUT3 in stage I nonsmall cell lung carcinoma is associated with poor survival, Cancer, 1997, 1046-1051). More recently, significantly higher GLUT1 and GLUT3 expression was found in poorly differentiated breast and endometrial (grade 2 and 3) tumours than in well-differentiated tumours (grade 1) (A. Krzeslak, K. Wojcik-Krowiranda, E. Forma, P. Jozwiak, H. Romanowicz, A. Bienkiewicz et al., Expression of GLUT1 and GLUT3 glucose transporters in endometrial and breast cancers, Pathol Oncol Res, 2012, 721-728). In addition, GLUT4, similar to GLUT1, displays an interesting connection with cancer, as both transporters are transcriptionally repressed by p53, a tumour suppressor protein important in cell cycle control and apoptosis, processes that are altered usually in cancer (F. Schwartzenberg-Bar-Yoseph, M. Armoni and E. Karnieli, The tumor suppressor p53 down-regulates glucose transporters GLUT1 and GLUT4 gene expression, Cancer Res, 2004, 2627-2633).

Up-regulation of glycolysis has also been demonstrated in a number of T and B cell driven leukemias such as AML, ALL (L. J. Akers, W. Fang, A. G. Levy, A. R. Franklin, P. Huang and P. A. Zweidler-McKay, Targeting glycolysis in leukemia: a novel inhibitor 3-BrOP in combination with rapamycin, Leuk Res, 2011, 814-820), Burkitt's lymphoma (A. Malenda, A. Skrobanska, T. Issat, M. Winiarska, J. Bil, B. Oleszczak et al., Statins impair glucose uptake in tumor cells, Neoplasia, 2012, 311-323), non-Hodgkins lymphoma and the related primary effusion lymphoma (A. P. Bhatt, S. R. Jacobs, A. J. Freemerman, L. Makowski, J. C. Rathmell, D. P. Dittmer et al., Dysregulation of fatty acid synthesis and glycolysis in non-Hodgkin lymphoma, Proc Natl Acad Sci USA, 2012, 11818-11823) and glycolytic inhibitors such as 2DG and 3-bromopyruvate have been shown to inhibit the growth of leukemia cells in culture (L. J. Akers, W. Fang, A. G. Levy, A. R. Franklin, P. Huang and P. A. Zweidler-McKay, Targeting glycolysis in leukemia: a novel inhibitor 3-BrOP in combination with rapamycin, Leuk Res, 2011, 814-820; A. P. Bhatt, S. R. Jacobs, A. J. Freemerman, L. Makowski, J. C. Rathmell, D. P. Dittmer et al., Dysregulation of fatty acid synthesis and glycolysis in non-Hodgkin lymphoma, Proc Natl Acad Sci USA, 2012, 11818-11823).

Hence, the inventors have determined that a strong rationale exists for the therapeutic utility of drugs which block the activity of SLC2A class I sugar transporters at a variety of solid and liquid cancers.

Immune (T and B) cell activation potently stimulates cellular metabolism to support the elevated energetic and biosynthetic demands of growth, proliferation, and effector function (V. A. Gerriets and J. C. Rathmell, Metabolic pathways in T cell fate and function, Trends Immunol, 2012, 168-173). Activation of effector T cells leads to increased glucose uptake, glycolysis, and lipid synthesis to support growth and proliferation. This increase in glucose metabolism is controlled by many of the same metabolic regulators that play an important role in cancer, including PI3K/mTOR, HIF1α, Myc and ERRα. Treg and memory CD8+ T cells instead mainly utilize fatty acids for energy. Regulation of glucose uptake and expression of GLUT1 was found to be limiting in T cell activation (S. R. Jacobs, C. E. Herman, N. J. Maciver, J. A. Wofford, H. L. Wieman, J. J. Hammen et al., Glucose uptake is limiting in T cell activation and requires CD28-mediated Akt-dependent and independent pathways, J Immunol, 2008, 4476-4486). Improperly controlled T cell metabolism can lead to chronic T cell activation and inflammatory disease. Indeed, direct manipulation of glucose metabolism in vivo has been shown to modulate inflammatory disease. GLUT1 is the primary glucose transporter in hematopoietic cells and is significantly up-regulated upon T cell activation. Overexpression of GLUT1 leads to increased glucose uptake and glycolysis, and transgenic expression of GLUT1 specifically in T cells leads to increased T cell proliferation, survival and cytokine production (S. R. Jacobs, C. E. Herman, N. J. Maciver, J. A. Wofford, H. L. Wieman, J. J. Hammen et al., Glucose uptake is limiting in T cell activation and requires CD28-mediated Akt-dependent and independent pathways, J Immunol, 2008, 4476-4486; R. D. Michalek, V. A. Gerriets, S. R. Jacobs, A. N. Macintyre, N. J. Maclver, E. F. Mason et al., Cutting edge: distinct glycolytic and lipid oxidative metabolic programs are essential for effector and regulatory CD4+ T cell subsets, J Immunol, 2011, 3299-3303). Targeting glucose metabolism has been shown to be effective to reduce T cell effector function. Treatment of mice with the glycolytic inhibitor 2-deoxyglucose suppressed experimental autoimmune encephalomyelitis (EAE) suggesting a potential role for the inhibition of SLC2A glucose transporters in autoimmune disorders such as multiple sclerosis (L. Z. Shi, R. Wang, G. Huang, P. Vogel, G. Neale, D. R. Green et al., HIF1alpha-dependent glycolytic pathway orchestrates a metabolic checkpoint for the differentiation of TH 17 and Treg cells, J Exp Med, 2011, 1367-1376). In humans, chronically activated T cells in allergic asthma patients have been shown to be highly glycolytically active producing high levels of lactate and overexpressing pyruvate dehydrogenase kinase 1 (PDK1). PDK1 acts to inhibit pyruvate dehydrogenase and thus restrict entrance of pyruvate into the mitochondrial citric acid cycle, instead promoting aerobic glycolysis and the production of lactic acid (M. Ostroukhova, N. Goplen, M. Z. Karim, L. Michalec, L. Guo, Q. Liang et al., The role of low-level lactate production in airway inflammation in asthma, Am J Physiol Lung Cell Mol Physiol, 2012, L300-307). Treatment of CD4+ T cells isolated from asthma patients with the PDK1 inhibitor dichloroacetate (DCA) promotes pyruvate oxidation in the mitochondria and prevented inflammatory cytokine production and T cell proliferation. As in human patients, T cells from mice in models of asthma produce high levels of lactate. Treatment of these mice with DCA reduced lactate production and inhibited airway inflammation in vivo. Inhibition of aerobic glycolysis with DCA also inhibited collagen-induced arthritis in female mice (L. Bian, E. Josefsson, I. M. Jonsson, M. Verdrengh, C. Ohlsson, M. Bokarewa et al., Dichloroacetate alleviates development of collagen II-induced arthritis in female DBA/1 mice, Arthritis Res Ther, 2009, R132). The differences in metabolism between effector and regulatory T cells may provide an opportunity to modulate the balance between effector and regulatory T cells or to inhibit autoreactive and inflammatory T cells with minimal effect on healthy lymphocytes. Targeting T cell metabolism may, therefore, provide new directions to modulate the immune response and treat an array of inflammatory diseases or to potentially impact T cell responses to infection.

Hence, the inventors have determined that a strong rationale exists for the therapeutic utility of drugs which block the activity of SLC2A class I sugar transporters at reducing aberrant T and B cell immune responses in inflammatory and autoimmune conditions.

Recently, it was demonstrated that cells from polycystic kidney disease patients exhibited elevated levels of glycolysis and inhibition of glycolysis resulted in improved kidney function in mouse models of this chronic progressive disease (I. Rowe, M. Chiaravalli, V. Mannella, V. Ulisse, G. Quilici, M. Pema et al., Defective glucose metabolism in polycystic kidney disease identifies a new therapeutic strategy, Nat Med, 2013, 488-493). In addition, GLUT1 expression is increased in hepatocellular carcinoma (HCC), where GLUT1 acts as a tumour promoter. Hyperglycemia is one of the factors known to induce and promote hepatic fibrogenesis, and the activation of hepatic stellate cells (HSCs) is the key event of hepatic fibrosis. GLUT1 suppression has been shown to impair glucose uptake and lactate secretion of HSCs indicative for reduced anaerobic glycolysis. Functional analysis demonstrated that reduced GLUT1 expression using siRNA led to reduced glucose uptake, lactate secretion and lower apoptosis resistance of HSCs (B. Czech, D. Valletta, M. Saugspier, M. Müller, A. K. Bosserhoff and C. Hellerbrand, Effect of increased glucose transporter 1 (GLUT1) expression in activated hepatic stellate cells, Z Gastroenterol, 2013, P_1_10). Finally, intimal hyperplasia is characterized by exaggerated proliferation of vascular smooth muscle cells (VSMCs). Enhanced VSMC growth is dependent on increased glucose uptake and metabolism. Studies have demonstrated that GLUT1 overexpression contributes to phenotypic changes in VSMCs (R. Pyla, N. Poulose, J. Y. Jun and L. Segar, Expression of conventional and novel glucose transporters, GLUT1, -9, -10, and -12, in vascular smooth muscle cells, Am J Physiol Cell Physiol, 2013, C574-589).

Hence, the inventors have determined that a strong rationale exists for the therapeutic utility of drugs which block the activity of SLC2A class I sugar transporters, in particular GLUT1, at treating proliferative disorders such as intimal hyperplasia, chronic kidney and liver disease including fibrosis and cirrhosis.

Metabolic regulation of neuronal excitability is recognized as a factor in the pathogenesis and control of seizures. Inhibiting or bypassing glycolysis may be one way through which the ketogenic diet provides an anticonvulsant effect. 2-deoxy-D-glucose (2DG), a nonmetabolizable glucose analog that partially inhibits glycolysis. 2DG has antiepileptic effects by retarding the progression of kindled seizures (C. E. Stafstrom, J. C. Ockuly, L. Murphree, M. T. Valley, A. Roopra and T. P. Sutula, Anticonvulsant and antiepileptic actions of 2-deoxy-D-glucose in epilepsy models, Ann Neurol, 2009, 435-447; C. E. Stafstrom, A. Roopra and T. P. Sutula, Seizure suppression via glycolysis inhibition with 2-deoxy-D-glucose (2DG), Epilepsia, 2008, 97-100).

Therefore, the inventors have determined that a strong rationale exists for the therapeutic utility of drugs which block the activity of SLC2A class I sugar transporters, in particular GLUT1 and GLUT3, in treating epilepsy.

There is an established body of work investigating the utility of fused bicyclic imidazolyl compounds and related compounds in medicine. In WO 2012/130322 Elara Pharmaceuticals describe imidazopyridine compounds that inhibit Hypoxia Inducible Factor (HIF)-medicated transcription and signalling under hypoxic conditions. The compounds are suggested to be of use in the treatment or prevention of diseases including inflammatory disease, a hyperproliferative disease or disorder, a hypoxia-related pathology and a disease characterized by excessive vascularization. Sanghani et al. describe the preparation of a ten imidazopyridines and their antibacterial and antifungal activities in Archives of Applied Science Research, 2010, 2, 444-450. In European Journal of Medicinal Chemistry, 2010, 45, 5208-5216 Myadaraboina et al. describe SAR studies around imidazopyrazine molecules which show activity against cancer cell lines. The authors conclude that bromine substitution at R7 is important for activity against cancer cell lines. Almirante et al (Journal of Medicinal Chemistry, 1965, 8, 305-312) describe imidazopyridines with analgesic, anti-inflammatory, antipyretic and anticonvulsant activity. WO 2008/116665 from Santhera Pharmaceuticals describes imidazopyridines bearing amide substituents at the R3 position as melanocortin-4 receptor antagonists that may be of use for the treatment of diseases such as cancer cachexia, muscle wasting, anorexia, amytrophic lateral sclerosis, anxiety and depression. WO 2009/143156 from Sepracor describes imidazopyridines as GABAA receptor modulators that may therefore be of use in the treatment of various conditions, including anxiety. EP 172096A from Synthelabo describes imidazopyridines that may be of use as anxiolytics, anticonvulsants or for treating other CNS disorders. However, these disclosures are concerned with different mechanisms than those currently of interest to the present inventors. Thus, inhibition of GLUT1 is not studied, or considered, in any of these references.

The inventors have determined that a strong rationale exists for the therapeutic utility of fused bicyclic imidazolyl compounds, and similar related compounds, which block the activity of SLC2A class I sugar transporters, in particular GLUT1.

Thus, it is an aim of the present invention to provide SLC2A class I transporter inhibitors, and in particular GLUT1, GLUT2, GLUT3, GLUT4 and GLUT14 inhibitors. It is especially an aim to provide SLC2A class I transporter inhibitors, such as GLUT1 GLUT2, GLUT3, GLUT4 and GLUT14 inhibitors for use in medicine. It is a further aim to provide pharmaceutical compositions comprising such inhibitors, and in particular to provide compounds and pharmaceutical compositions for treating a cancer, an inflammatory condition, an autoimmune condition, a neurological condition, a proliferative disorder, and/or a metabolic condition. It is also an aim to provide methods of synthesis of the inhibitor compounds, and methods of screening for new SLC2A class I transporter inhibitors.

Accordingly, the present invention provides a SLC2A class I transporter inhibitor compound for use in medicine, which compound comprises the following formula:

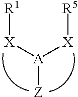

wherein A and Z may be the same or different and are each independently selected from C and N; each X may be the same or different and is independently selected from C, N, O and S; R¹ and R⁵ may be present or absent and may be the same or different and are each selected from H and a substituted or unsubstituted organic group; R¹ and R⁵ may together form a substituted or unsubstituted saturated or unsaturated aliphatic or aromatic homocyclic or heterocyclic ring; Z completes a ring with each X, each ring comprising from 3 to 8 ring atoms including the X, A, and Z, each ring atom being independently selected from C, N, O and S, and each ring atom being unsubstituted or independently substituted with H or a substituted or unsubstituted organic group; and wherein the bonds between all of the atoms in the rings including the X, A, and Z may independently be single bonds or double bonds, provided that when X or a ring atom is O or S the bonds to X are single bonds.

In the above structure, when A is C it may comprise a further substituent selected from H or an organic group, or alternatively it may be double bonded to one X. This further substituent may, together with either R¹ and/or R⁵, form a substituted or unsubstituted saturated or unsaturated aliphatic or aromatic homocyclic or heterocyclic ring. As with rings formed together by R¹ and R⁵, the homocyclic ring is typically a carbocyclic ring.

In the context of the present invention, a compound is considered to be a SLC2A Class I transporter inhibitor if it is capable of reducing transport of a species (such as glucose) across an SLC2A Class I transporter receptor (such as GLUT1, GLUT2, GLUT3, GLUT4 and/or GLUT14) (e.g. into or out of a cell having a SLC2A Class I transporter receptor on its surface) as compared with transport of the same species in the absence of the compound.

The compounds are thus compounds with a fused ring system comprising two rings fused through the A and Z atoms. The curved lines each represent the completion of each ring of the system between the Z and the X atoms. Each ring in the system may comprise from 3 to 8 ring atoms. When a ring comprises three ring atoms, its X and Z atoms are directly bonded to each other. When a ring has 4, 5, 6, 7 or 8 atoms, its X and Z atoms are bonded via 1, 2, 3, 4 or 5 further ring atoms respectively. These further ring atoms may be selected from C, N, O and S, and may be joined to each other by single or double bonds depending upon the nature of each ring atom and whether it possesses a substituent. The ring atoms are typically selected from C and N atoms, and more typically in the left ring all ring atoms are C whilst in the right ring one of the ring atoms or X is N and the rest of the atoms in the ring are C. Alternatively, in some embodiments, in each of the two rings one of the ring atoms or X is N and the rest of the atoms in the ring are C. In preferred embodiments, the number of atoms in each ring of the fused ring system are 3:3, 3:4, 3:5, 3:6, 3:7, 3:8, 4:3, 4:4, 4:5, 4:6, 4:7, 4:8, 5:3, 5:4, 5:5, 5:6, 5:7, 5:8, 6:3, 6:4, 6:5, 6:6, 6:7, 6:8, 7:3, 7:4, 7:5, 7:6, 7:7, 7:8, 8:3, 8:4, 8:5, 8:6, 8:7, and 8:8, where in this notation the first number represents the left-hand ring of the above system and the second number represents the right-hand ring of the system. It is more preferred that the ring system is 5:5, 5:6, 5:7, 6:5, 6:6, 6:7, 7:5, 7:6 and 7:7 and 5:6 and 6:5 systems are the most preferred.

In particularly preferred embodiments the ring system is planar (i.e. both rings are co-planar with each other). In such compounds, A is typically N or a C which is double-bonded to one of the X groups. In such embodiments, typically both rings are aromatic, although in other embodiments one or both of the rings may be non-aromatic (such as unsaturated rings) and co-planar.

In all of the embodiments mentioned in connection with this invention, both above and in the following, the substituents are selected from H and an organic group. Thus, both above and in the following, the terms ‘substituent’ and ‘organic group’ are not especially limited and may be any functional group or any atom, especially any functional group or atom common in organic chemistry. Thus, ‘substituent’ and ‘organic group’ may have any of the following meanings.

The substituent may comprise any organic group and/or one or more atoms from any of groups IIIA, IVA, VA, VIA or VIIA of the Periodic Table, such as a B, Si, N, P, O, or S atom (e.g. OH, OR, NH₂, NHR, NR₂, SH, SR, SO₂R, SO₃H, PO₄H₂) or a halogen atom (e.g. F, Cl, Br or I) where R is a linear or branched lower hydrocarbon (1-6 C atoms) or a linear or branched higher hydrocarbon (7 C atoms or more, e.g. 7-40 C atoms).

When the substituent comprises an organic group, the organic group preferably comprises a hydrocarbon group. The hydrocarbon group may comprise a straight chain, a branched chain or a cyclic group. Independently, the hydrocarbon group may comprise an aliphatic or an aromatic group. Also independently, the hydrocarbon group may comprise a saturated or unsaturated group.

When the hydrocarbon comprises an unsaturated group, it may comprise one or more alkene functionalities and/or one or more alkyne functionalities. When the hydrocarbon comprises a straight or branched chain group, it may comprise one or more primary, secondary and/or tertiary alkyl groups. When the hydrocarbon comprises a cyclic group it may comprise an aromatic ring, an aliphatic ring, a heterocyclic group, and/or fused ring derivatives of these groups. The cyclic group may thus comprise a benzene, naphthalene, anthracene, indene, fluorene, pyridine, quinoline, pyrrolidine, piperidine, morpholine, thiophene, benzothiophene, furan, benzofuran, pyrrole, indole, imidazole, thiazole, and/or an oxazole group, as well as regioisomers of the above groups.

The number of carbon atoms in the hydrocarbon group is not especially limited, but preferably the hydrocarbon group comprises from 1-40 C atoms. The hydrocarbon group may thus be a lower hydrocarbon (1-6 C atoms) or a higher hydrocarbon (7 C atoms or more, e.g. 7-40 C atoms). The lower hydrocarbon group may be a methyl, ethyl, propyl, butyl, pentyl or hexyl group or regioisomers of these, such as isopropyl, isobutyl, tert-butyl, etc. The number of atoms in the ring of the cyclic group is not especially limited, but preferably the ring of the cyclic group comprises from 3-10 atoms, such as 3, 4, 5, 6, 7, 8, 9 or 10 atoms.

The groups comprising heteroatoms described above, as well as any of the other groups defined above, may comprise one or more heteroatoms from any of groups IIIA, IVA, VA, VIA or VIIA of the Periodic Table, such as a B, Si, N, P, O, or S atom or a halogen atom (e.g. F, Cl, Br or I). Thus the substituent may comprise one or more of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, sulphonyl groups, and phosphate groups etc. The substituent may also comprise derivatives of these groups, such as carboxylic acid anhydrides and carboxylic acid halides.

In addition, any substituent may comprise a combination of two or more of the substituents and/or functional groups defined above.

The invention will now be explained in more detail, by way of example only, with reference to the following Figure.

FIG. 1 shows a phylogenetic tree showing the relationship between the human SLC2A gene family for all 14 members. Distance between branches and length of the lines indicates the degree of evolutionary divergence. From Manolescu et al (2007) Physiology 22:234-240.

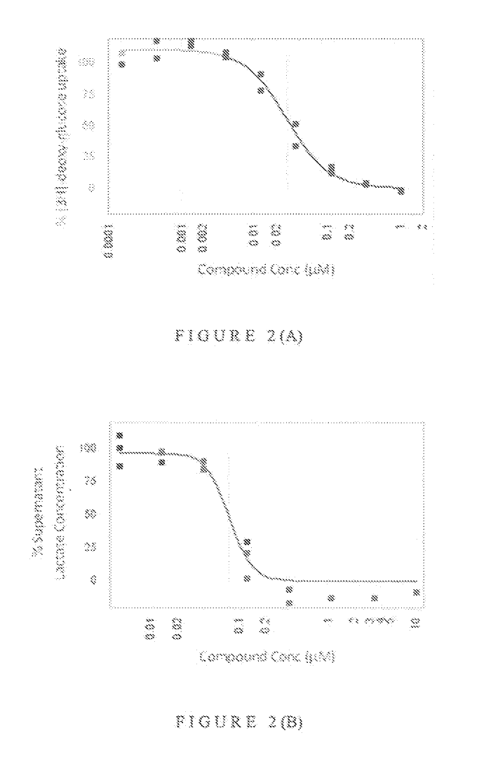

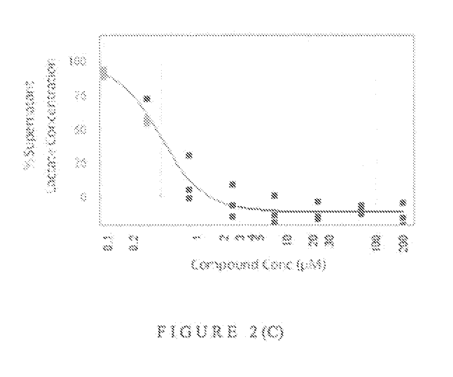

FIG. 2 shows: (A) inhibition, by compound 155 of [3H]-deoxy-D-glucose uptake in HEK293 cells overexpressing human GluT1; (B,C) inhibition of lactate secretion from A549 cells cultured in (B) 5 mM or (C) 17 mM glucose. Lactate levels in the supernatant were assayed after 4 hours of exposure to compound 155 at the indicated concentrations.

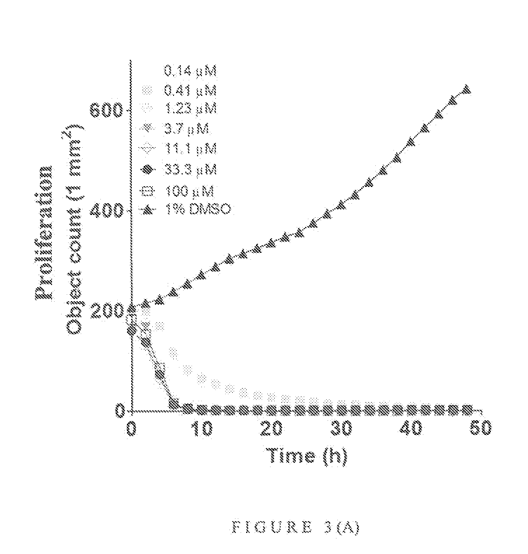

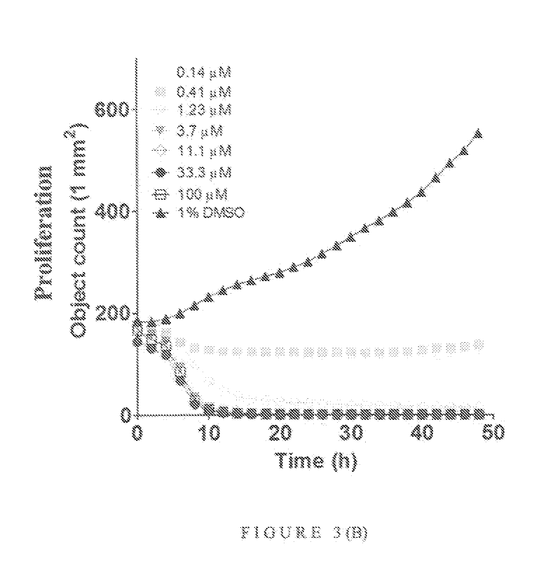

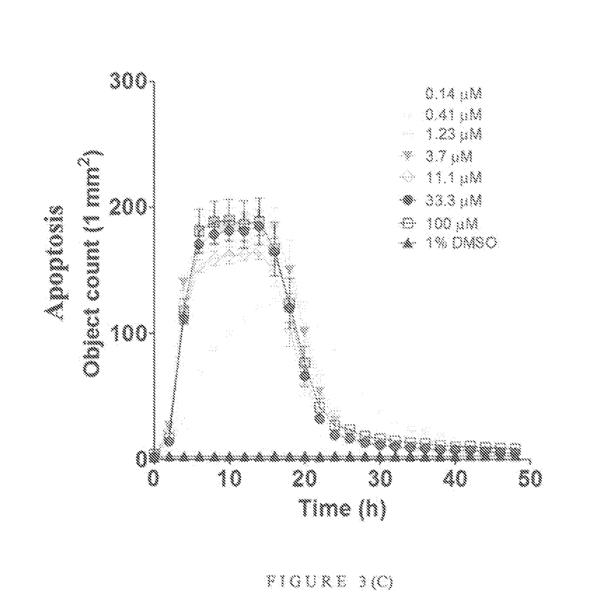

FIG. 3 shows: (A,B) cell proliferation measurement in cells; (C,D) apoptosis induction measurement in cells.

The invention will now be described in more detail.

As has been described, the invention relates to a SLC2A class I transporter inhibitor compound for use in medicine, which compound comprises the following formula:

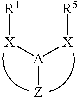

wherein A and Z may be the same or different and are each independently selected from C and N; each X may be the same or different and is independently selected from C, N, O and S; R¹ and R⁵ may be present or absent and may be the same or different and are each selected from H and a substituted or unsubstituted organic group; R¹ and R⁵ may together form a substituted or unsubstituted saturated or unsaturated aliphatic or aromatic homocyclic or heterocyclic ring; Z completes a ring with each X, each ring comprising from 3 to 8 ring atoms including the X, A, and Z, each ring atom being independently selected from C, N, O and S, and each ring atom being unsubstituted or independently substituted with H or a substituted or unsubstituted organic group; and wherein the bonds between all of the atoms in the rings including the X, A, and Z may independently be single bonds or double bonds, provided that when X or a ring atom is O or S the bonds to X are single bonds.

The number of substituents on an X or a ring atom will depend on its valency. Thus, it will be apparent in all of the embodiments of the invention, both above and below, that when X or a ring atom has only single bonds, it will have no substituents if it is O or S, 1 substituent (H or an organic group as defined herein) if it is N, and 2 substituents (each independently chosen from H or an organic group as defined herein) if it is C.

The Z and A atoms are not especially limited and may be the same or different. Thus both Z and A may be carbon atoms, or both Z and A may be nitrogen atoms. However, typically one of Z or A is N, and more preferably Z is N. In the most preferred embodiments, Z is N and A is C.

Thus, in certain embodiments, the compound comprises the following formula:

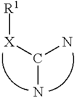

wherein R¹, the rings, and X are as defined above; and wherein the bonds between all of the atoms in the rings including the X, C, and N may independently be single bonds or double bonds.

In more typical embodiments, the compound comprises the following formula:

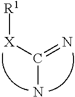

wherein R¹, the rings, and X are as defined above; and wherein there is a double bond between a C and N as shown and otherwise the bonds between all of the atoms in the rings including the X, C, and N may independently be single bonds or double bonds.

In certain embodiments the compound may comprise a formula selected from one of the following:

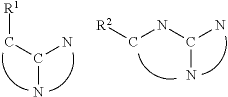

wherein R¹ and the rings are as defined above; R² is selected from H and a substituted or unsubstituted organic group; and wherein the bonds between all of the atoms in the rings including the C and N may independently be single bonds or double bonds.

In more typical embodiments, the compound comprises a formula selected from one of the following:

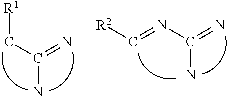

wherein R¹ and the rings are as defined above; R² is selected from H and a substituted or unsubstituted organic group; and wherein there is a double bond between one C and N, or between two C and N, as shown and otherwise the bonds between all of the atoms in the rings including the C and N may independently be single bonds or double bonds.

In certain embodiments, the compound comprises a formula selected from one of the following:

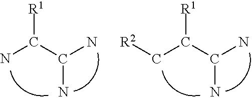

wherein R¹ and the rings are as defined above; R¹ and R² may be the same or different; R² is selected from H and a substituted or unsubstituted organic group; wherein R¹ and R² may together form a substituted or unsubstituted saturated or unsaturated aliphatic or aromatic homocyclic or heterocyclic ring; and wherein the bonds between all of the atoms in the rings including the C and N may independently be single bonds or double bonds.

In still more typical embodiments, the compound comprises a formula selected from one of the following:

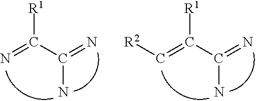

wherein R¹ and the rings are as defined above; R¹ and R² may be the same or different and are as defined above; and wherein there is a double bond between two Cs and Ns, or between one C and C and one C and N, as shown and otherwise the bonds between all of the atoms in the rings including the C and N may independently be single bonds or double bonds. In some embodiments, R¹ and R² may together form a substituted or unsubstituted saturated or unsaturated aliphatic or aromatic homocyclic or heterocyclic ring. Such rings may be of any of the specific types defined above in relation to substituents.

In certain embodiments the compound may comprise a formula selected from one of the following:

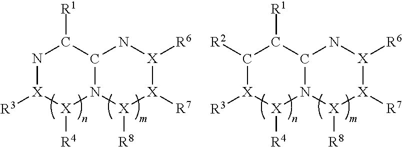

wherein R¹, R², R³, R⁴, R⁶, R⁷ and R⁸ may be the same or different and are independently selected from H and a substituted or unsubstituted organic group; each X is selected from C, N, O and S; when X is O or S the corresponding R group is absent; adjacent R groups may together form a substituted or unsubstituted saturated or unsaturated aliphatic or aromatic homocyclic or heterocyclic ring; n is an integer of 0, 1 or 2; and m is an integer of 0, 1 or 2; and wherein the bonds between all of the atoms in the rings including the X, C and N may independently be single bonds or double bonds; preferably wherein each X is C; and/or preferably wherein n=1; and/or preferably wherein m=0; and/or preferably wherein R¹ is H; and/or preferably wherein R⁴ is H or Me; and/or preferably wherein R³, R⁶ and R⁷ are not H.

In further embodiments, the compound may comprise a formula selected from one of the following:

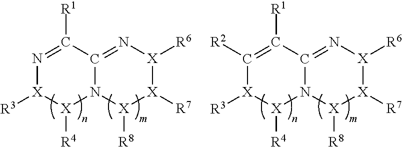

wherein R¹, R², R³, R⁴, R⁶, R⁷ and R⁸ may be the same or different and are independently selected from H and a substituted or unsubstituted organic group; adjacent R groups may together form a substituted or unsubstituted saturated or unsaturated aliphatic or aromatic homocyclic or heterocyclic ring; each X is selected from C, N, O and S; n is an integer of 0, 1 or 2; and m is an integer of 0, 1 or 2; and wherein there is a double bond between two Cs and Ns, or between one C and C and one C and N, as shown and otherwise the bonds between all of the atoms in the rings including the X, C and N may independently be single bonds or double bonds.

Thus, the value of n and m determine the nature of the ring system. In more typical embodiments each X is C, although in some embodiments the X in the left-hand ring is N whilst the X in the right-hand ring is absent or is C. Thus, typically n=1 and m=0, although in some embodiments n=2 and m=0, n=2 and m=1, n=2 and m=2, n=1 and m=1, n=1 and m=2, n=0 and m=0, n=0 and m=1 or n=0 and m=2. In more typical embodiments R¹ is H; and/or R is H or Me. In addition to this, or alternative to this, it is typical that all of R³, R⁶ and R⁷ are not H.

In some compounds, one or more of the following adjacent R groups together form a ring as defined above: R¹ and R²; R² and R³; R³ and R⁴; R⁴ and R⁸; R⁸ and R⁷; and R⁷ and R⁶. Typically the ring may be a substituted or unsubstituted saturated or unsaturated aliphatic or aromatic homocyclic or heterocyclic ring. As with rings defined above, the homocyclic ring is typically a carbocyclic ring.

In yet more typical embodiments, the compound comprises a formula selected from one of the following:

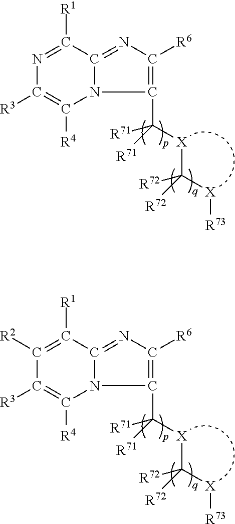

wherein each R⁷¹, each R⁷² may be the same or different and are independently selected from H and a substituted or unsubstituted organic group; two R⁷¹ groups and/or two R⁷² groups may together form a carbonyl group; each X is selected from C, N, O and S; R⁷³ is absent when its X atom is O or S and may be the same or different as R⁷¹ and R⁷² and is independently selected from H and a substituted or unsubstituted organic group; p is an integer of from 0 to 6; and q is an integer of from 0 to 6; and optionally wherein the X groups may complete a ring with each other, each ring atom being the same or different and being independently selected from C, N, O and S, and each ring atom being unsubstituted or independently substituted with H or a substituted or unsubstituted organic group, and wherein the bonds between all of the atoms in the optional ring including X atoms and C atoms may independently be single bonds or double bonds; and wherein an R⁷² and R⁷³ may together form a substituted or unsubstituted saturated or unsaturated aliphatic or aromatic homocyclic or heterocyclic ring as defined above.

Thus p and q determine the distribution of the X atoms relative to the ring system. Whilst p may be 0, 1, 2, 3, 4, 5 or 6, more typically p is 1, 2 or 3 and most typically p is 1. Similarly, whilst q may be 0, 1, 2, 3, 4, 5 or 6, more typically q is 1, 2, or 3 and most typically q is 2.

The dotted curved line represents an optional ring system incorporating both of the X atoms and the carbon atom or atoms associated with q. In typical embodiments this ring system is present. This ring system may comprise from 3 to 8 ring atoms. When a ring comprises three ring atoms, the two X atoms are directly bonded to each other via the dotted ring and q=1, or the two X atoms are directly bonded to each other with q=0 and the ring completed by a further ring atom that may be C, N, O or S and may be unsubstituted or substituted with one or more R⁷² groups. When a ring has 4, 5, 6, 7 or 8 atoms, the two X atoms are bonded via 1, 2, 3, 4, 5 or 6 further ring atoms, depending upon the value of q. As mentioned above, these X atoms may be selected from C, N, O and S, and may be substituted by one or more R⁷ groups. All of the atoms in the ring system may be joined to each other by single or double bonds depending upon the nature of each ring atom and whether it possesses a substituent. However, in typical embodiments, all of the bonds are single bonds.

In more typical embodiments, the X atom closest to the fused ring system is N and the X atom furthest from the fused ring system is selected from N, O and S. In other typical embodiments the X atoms complete a six membered ring in which all ring bonds are single bonds.

When the optional ring is absent, then q is typically 1, 2 or 3 and the X atom closest to the fused ring system is N and the X atom furthest from the fused ring system is selected from N, O and S.

The R⁷³ group is not especially limited, but typically comprises any substituent comprising a substituted or unsubstituted group comprising a carbonyl group. Thus the R⁷³ group may be a group such as —(CO)H, —(CO)R, —CH₂(CO)H and —CH₂(CO)R, where R is an organic group as defined above.

In all of the embodiments above, it is typical that R¹ is H, R² is H, R⁴ is H or Me, R³ is not H, R⁶ is not H, each R⁷¹ is H, and each R⁷² is H.

In some compounds, one or more of the following adjacent R groups together form a ring as defined above: R¹ and R²; R² and R³; R³ and R⁴; R⁷¹ and R⁷²; and R⁷² and R⁷³. Typically the ring may be a substituted or unsubstituted saturated or unsaturated aliphatic or aromatic homocyclic or heterocyclic ring. As with rings defined above, the homocyclic ring is typically a carbocyclic ring.

In still more typical embodiments, the compound comprises a formula selected from one of the following:

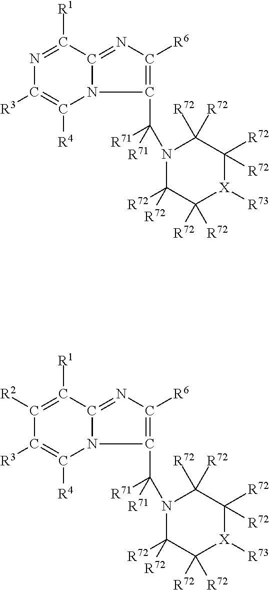

wherein X is independently selected from N, O and S; wherein R⁷³ is absent when its X is O or S and is selected from H and a substituted or unsubstituted organic group, as defined above.

As mentioned above, the R⁷³ group is not especially limited, and may comprise any substituted or unsubstituted group comprising a carbonyl group. Thus, the R⁷³ group may be a group such as —(CO)H, —(CO)R, —CH₂(CO)H and —CH₂(CO)R, where R is an organic group as defined above, and in particular may comprise an unsubstituted or substituted acyl group.

In the above embodiment it is typical that X is N.

In all of the embodiments above, it is typical that R¹ is H, R² is H, R⁴ is H or Me, R³ is not H, R⁶ is not H, each R⁷¹ is H, and each R⁷² is H.

In some compounds, one or more of the following adjacent R groups together form a ring as defined above: R¹ and R²; R² and R³; R³ and R⁴; R⁷¹ and R⁷²; and R⁷² and R⁷³. Typically the ring may be a substituted or unsubstituted saturated or unsaturated aliphatic or aromatic homocyclic or heterocyclic ring. As with rings defined above, the homocyclic ring is typically a carbocyclic ring.

In yet further typical embodiments, the compound comprises a formula selected from one of the following:

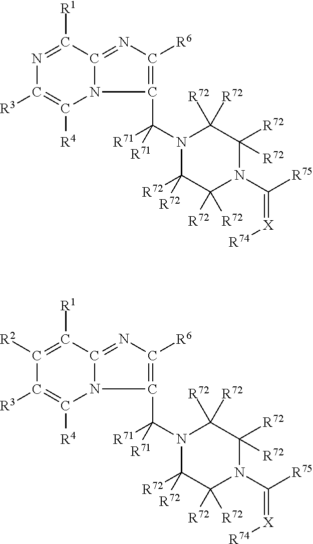

wherein X is independently selected from N, O and S; R⁷⁴ is absent when X is O or S and is selected from H and a substituted or unsubstituted organic group when X is N; and R⁷⁵ is selected from H and a substituted or unsubstituted organic group.

In more typical embodiments X is O.

Typically the R⁷⁵ group is selected from a substituted or unsubstituted linear or branched alkyl group, and an aliphatic or aromatic saturated or unsaturated homocyclic (such as carbocyclic) or heterocyclic ring such as a cycloalkyl group, a saturated or unsaturated heterocyclic group, and an aryl group as defined above. Particularly preferred aryl groups include substituted or unsubstituted phenyl groups or heterocyclic groups as defined above.

In all of the embodiments above, it is typical that R¹ is H, R² is H, R⁴ is H or Me, R³ is not H, R⁶ is not H, each R⁷ is H, and each Rn is H.

In some compounds, one or more of the following adjacent R groups together form a ring as defined above: R¹ and R²; R² and R³; R³ and R⁴; and R⁷¹ and R⁷². Typically the ring may be a substituted or unsubstituted saturated or unsaturated aliphatic or aromatic homocyclic or heterocyclic ring. As with rings defined above, the homocyclic ring is typically a carbocyclic ring.

Further typical compounds of the invention comprises a formula selected from one of the following:

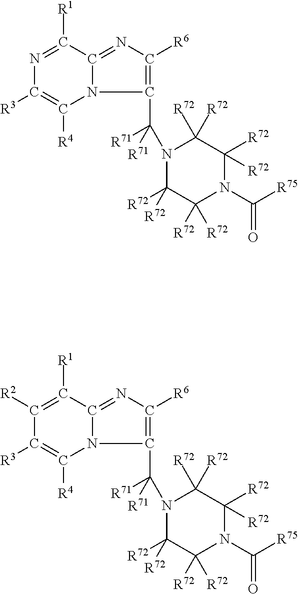

wherein R⁷⁵ is a substituted or unsubstituted organic group selected from: a linear or branched alkyl group, a cycloalkyl group, a saturated or unsaturated heterocyclic group, and an aryl group; preferably wherein R¹ is H, R² is H, R⁴ is H or Me, R³ is not H, R⁶ is not H, each R⁷¹ is H, each R⁷² is H;

preferably wherein R⁷⁵ is a group having the following structure:

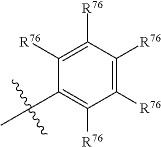

wherein each R⁷⁶ may be the same or different and is independently selected from H and a substituted or unsubstituted organic group.

In further typical embodiments, R¹ is H, R² is H, R⁴ is H or Me, R³ is not H, R⁶ is not H, each R⁷¹ is H, each R⁷² is H, and at least one of R⁷⁶ is not H.

In some compounds, one or more of the following adjacent R groups together form a ring as defined above: R¹ and R²; R² and R³; R³ and R⁴; and R⁷¹ and R⁷². Typically the ring may be a substituted or unsubstituted saturated or unsaturated aliphatic or aromatic homocyclic or heterocyclic ring. As with rings defined above, the homocyclic ring is typically a carbocyclic ring.

In respect of the above, in typical embodiments the following compounds are preferred:

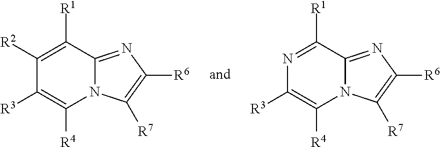

wherein R¹, R², R³, R⁴, R⁵, R⁶ and R⁷ are as described anywhere herein.

Especially preferred are compounds in which R¹, R², and R⁴ are all H:

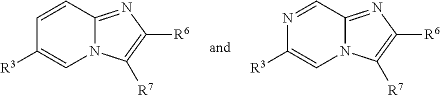

wherein R³, R⁶ and R⁷ are as described anywhere herein.

The compounds of the present invention have been described above with reference to a number of differing formulae. In the following, the substituents referred above and in particular referred to in each of the formulae will be described.

In some embodiments, the R³ group is preferably not H and also preferably not Me and also preferably not Et, and also preferably not an amido group of the form —CO—NRR′ (where R and R′ may be the same or different and are H or organic groups). However, in typical embodiments R³ may be a group selected from the following groups:

A halogen (such as F, Cl, Br and I).

A linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl), preferably a C₃-C₆ alkyl group (such as propyl (Pr), iso-propyl (i-Pr), n-butyl (n-Bu), iso-butyl (i-Bu), tert-butyl (t-Bu), pentyl and hexyl).

A linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph). In this notation Ph means phenyl, (2,3 or 4)F-Ph means a phenyl groups substituted by F at either the 2-, 3- or 4-position.

A linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃).

A linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt).

A amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph. In this notation 2,(3,4,5 or 6)F₂-Ph means a phenyl group substituted by one F at the 2-position and a second F at either the 3, 4, 5, or 6 position. Where there are two substituents the may also be in the 3,(4 or 5) position if desired.

A cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl).

A cyclic C₃-C₅ alkyl group (such as cyclopropyl (cyPr), cyclobutyl (cyBu), cyclopentyl, cyclohexyl, cycloheptyl and cyclooctyl).

A linear or branched C₁-C₆ alcohol group (such as —CH₂OH, —CH₂CH₂OH, —CH₂CH₂CH₂OH, —CH₂CH₂CH₂CH₂OH, —CH₂CH₂CHCH₂CH₂OH, and —CH₂CH₂CH₂CH₂CH₂CH₂OH).

A linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH).

A linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂.

A linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe).

A linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt).

A linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph.

A linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe).

A linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂.

A sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃.

A sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe).

An aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃).

A cyclic aminosulphonyl-group (such as —N(SO₂)(CH₂)₃ and —N(SO₂)(CH₂)₄).

An aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph- , 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-).

A saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

Especially preferred R³ groups include the following:

An unsubstituted, 2-monosubstituted, or 2,6-disubstituted phenyl group, preferably where the substituent is selected from F, Cl and CN (such as Ph-, 2-F-Ph-, 2,6-F₂-Ph-, 2-Cl-Ph-, 2,6-Cl₂-Ph-, 2-CN-Ph-, 2,6-(CN)₂-Ph-, 2,6-F, CN-Ph-, 2,6-F, Cl-Ph-, and 2,6-Cl, CN-Ph-).

An unsubstituted or 3-monosubstituted pyridine-2-yl, or a 2,6-disubstituted pyridine-4-yl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂—Pyr-4-yl. In this context Pyr means pyridine.

An unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl).

A substituted or unsubstituted 1,2,4-oxadiazol-3-yl group.

A cyclic aminosulphonyl-group (such as —N(SO₂)(CH₂)₃ and —N(SO₂)(CH₂)₄).

A linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe.

A linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl).

A linear or branched sulphonyl group (such as —SO₂Me, —SO₂Et, —SO₂Pr, —SO₂iPr).

A thioether group (such as —SMe, —SEt, —SPr, and SiPr).

An isopropyl, cyclopropyl and a propen-2-yl group.

In typical embodiments R⁶ is not H. In such embodiments R⁶ may be a group selected from the following groups:

A halogen (such as F, Cl, Br and I).

A linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl).

A linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph). In this notation Ph means phenyl, (2,3 or 4)F-Ph means a phenyl groups substituted by F at either the 2-, 3- or 4-position.

A linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃).

A linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt).

A amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph. In this notation 2,(3,4,5 or 6)F₂-Ph means a phenyl group substituted by one F at the 2-position and a second F at either the 3, 4, 5, or 6 position. Where there are two substituents the may also be in the 3,(4 or 5) position if desired.

A cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl).

A cyclic C₃-C₅ alkyl group (such as cyclopropyl, cyclobutyl, cyclopentyl, cyclohexyl, cycloheptyl and cyclooctyl).

A linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH).

A linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH).

A linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂.

A linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe).

A linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt).

A linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph.

A linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe).

A linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂.

A sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃.

A sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe).

An aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃).

A cyclic aminosulphonyl-group (such as —N(SO₂)(CH₂)₃ and —N(SO₂)(CH₂)₄).

An aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph- , 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-).

A saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and fiuran-3-yl).

In particularly preferred compounds, preferably R⁶ comprises—an aromatic group selected from Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-

Especially preferred R⁶ groups include the following:

An unsubstituted, 2-, 3- or 4-monosubstituted, and a 2,4 or 3,4-disubstituted phenyl group, preferably wherein the substituent is F, Cl or —OMe (such as 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph).

A substituted or unsubstituted pyridine-2-yl, pyridine-3-yl or pyridine-4-yl group preferably wherein the substituent is F or Cl (such as pyridine-2-yl, pyridine-3-yl, pyridine-4-yl, 3-F-pyridine-2-yl, 3-Cl-pyridine-2-yl, 4-F-pyridine-2-yl, 4-Cl-pyridine-2-yl, 5-F-pyridine-2-yl, 5-Cl-pyridine-2-yl.

A cyclic ether group (such as tetrahydrofuran-2-yl, tetrahydrofuran-3-yl, terahydropyran-2-yl, tetrahydropyran-3-yl and tetrahydropyran-4-yl).

In typical embodiments R⁷ is not H. In such embodiments R⁷ is a group selected from the following groups:

A halogen (such as F, Cl, Br and I).

A linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl).

A linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph). In this notation Ph means phenyl, (2,3 or 4)F-Ph means a phenyl groups substituted by F at either the 2-, 3- or 4-position.

A linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃).

A linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt).

A amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph. In this notation 2,(3,4,5 or 6)F₂-Ph means a phenyl group substituted by one F at the 2-position and a second F at either the 3, 4, 5, or 6 position. Where there are two substituents the may also be in the 3,(4 or 5) position if desired.

A cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl).

A cyclic C₃-C₅ alkyl group (such as cyclopropyl, cyclobutyl, cyclopentyl, cyclohexyl, cycloheptyl and cyclooctyl).

A linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH).

A linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH).

A linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂.

A linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe).

A linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt).

A linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph.

A linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂C, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe).

A linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂.

A sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃.

A sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe).

An aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃).

A cyclic aminosulphonyl- group (such as —N(SO₂)CH₂)₃ and —N(SO₂)(CH₂)₄).

An aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph- , 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-).

A saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

In especially preferred embodiments, the R⁷ group is selected from the following:

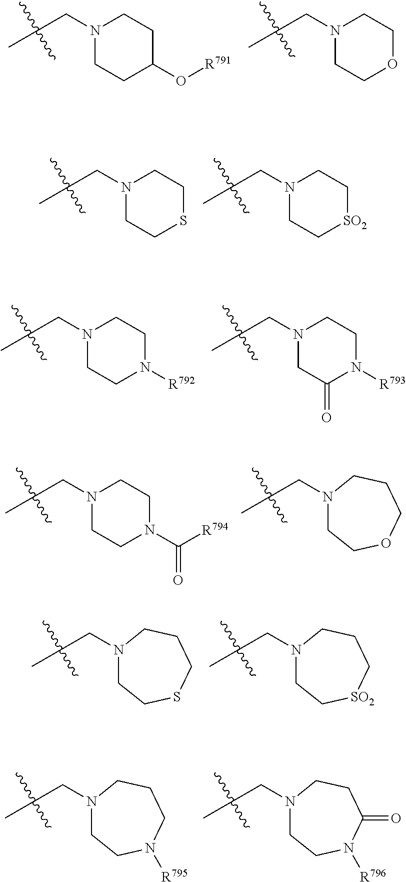

and also from a group having the following structure:

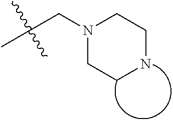 [SMILES: *CN1CCNCC1]

where the ring fused to the piperazine ring is a 5- or 6-membered ring optionally having 1 or more further heteroatoms in the ring selected from N and O and optionally having one or more substituents, preferably wherein this group is selected from the following:

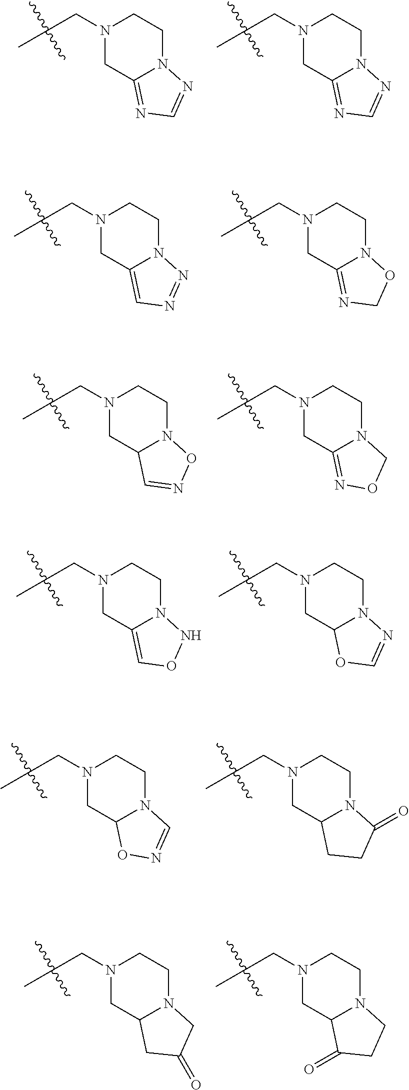

wherein R⁷⁹¹, R⁷⁹², R⁷⁹³, R⁷⁹⁴. R⁷⁹⁵, and R⁷⁹⁶ are selected from the substituents as defined herein, typically from the groups defined for R³ herein, and preferably for R⁷³ and R⁷⁵ herein, and especially preferably are selected from the following:

R⁷⁹¹ is selected from H, Me, Et, Pr, iPr, cyPr, and cyBu, preferably H.

R⁷⁹² is selected from H, Me, Et, Pr, iPr, cyPr, cyBu, —(CO)-Me, —(CO)-Et, —(CO)—Pr, —(CO)iPr, —SO₂Me, and SO₂Et, preferably H, Me, —SO₂Me and —(CO)-Me.

R⁷⁹³ is selected from H, Me, Et, Pr, iPr, cyPr, cyBu, —(CO)-Me, —(CO)Et, —(CO)Pr, and —(CO)iPr, preferably H, Me and —(CO)-Me.

R⁷⁹⁴ is selected from H, Me, Et, Pr, iPr, cyPr, cyBu, an unsubstituted, 2-, 3- or 4-monosubstituted, and a 2,4 or 3,4-disubstituted phenyl group, preferably wherein the substituent is F, ClCN, or —OMe (such as 2-F-Ph, 2-Cl-Ph, 2-CN-Ph, 2-OMe-Ph, 3-F-Ph, 3-Cl-Ph, 3-CN-Ph, 3-OMe-Ph, 4-F-Ph, 4-Cl-Ph, 4-CN-Ph, 4-OMe-Ph, 2,4-F₂-Ph, 2,4-Cl₂-Ph, 2,4-(CN)₂-Ph, 2,4-(OMe)₂-Ph, 3,4-F₂-Ph, 3,4-Cl₂-Ph, 3,4-(CN)₂-Ph, and 3,4-(OMe)₂-Ph); —NH₂, —NHMe, and —NMe₂; —OH, —OMe, —OEt, —OPr, —OiPr, —OⁿBu, and —O^{t}Bu; —CH₂OH, —CHMeOH, —C(Me)₂OH, —CH₂OMe, —CHMeOMe, —C(Me)₂OMe, —CH₂OEt, —CHMeOEt, and —C(Me)₂OEt; and a heterocyclic ring group having from 4-7 ring atoms with at least one heteroatom selected from N, O, and S, which ring group may be saturated or unsaturated and may be substituted or unsubstituted, preferably wherein the heterocyclic ring group is selected from the following groups:

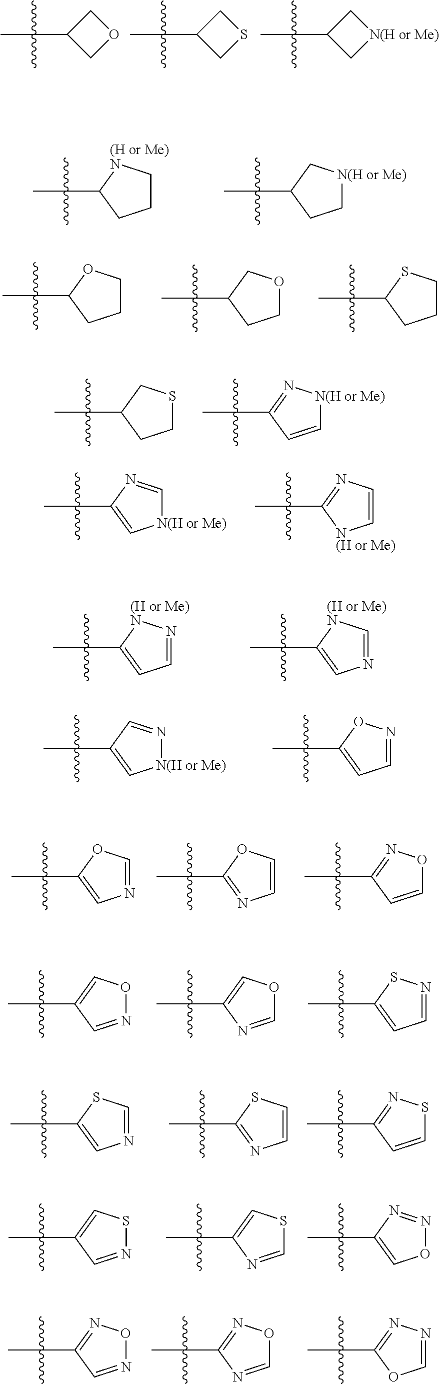

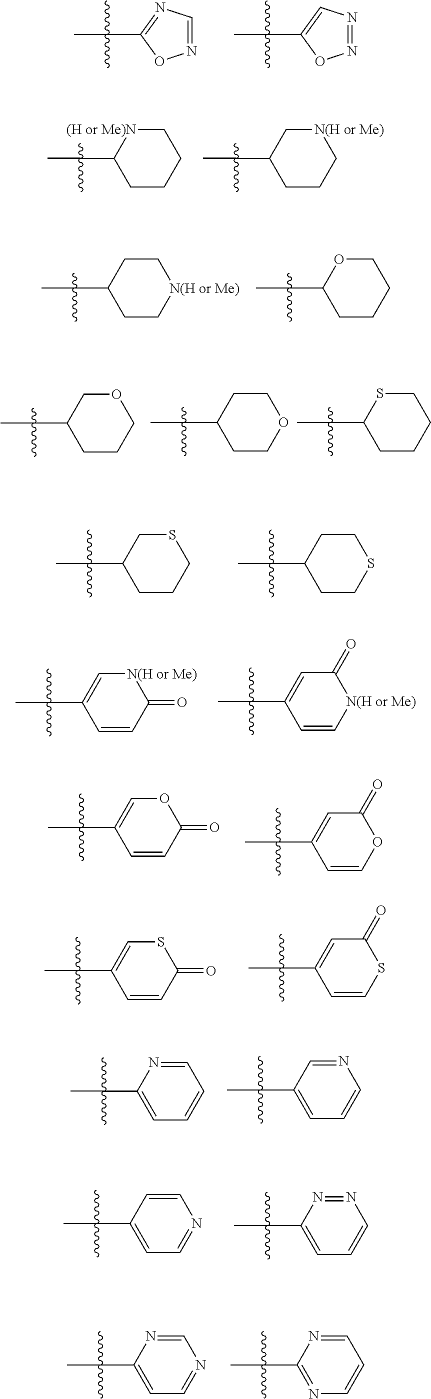

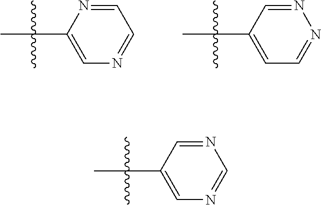 [SMILES: *c1cnccn1 ; *c1ccnnc1 ; *c1cncnc1]

R⁷⁹⁵ is selected from H, Me, Et, Pr, iPr, cyPr, cyBu, —(CO)-Me, —(CO)-Et, —(CO)—Pr, —(CO)iPr, —SO₂Me, and SO₂Et, preferably H, Me, —SO₂Me and —(CO)-Me.

R⁷ is selected from H, Me, Et, Pr, iPr, cyPr, cyBu, —(CO)-Me, —(CO)Et, —(CO)Pr, and —(CO)iPr, preferably H, Me and —(CO)-Me.

In typical embodiments R¹, R², R⁴, R⁵, and R⁸ are all H. Alternatively each may be independently H or a group selected from the following groups:

A halogen (such as F, Cl, Br and I).

A linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl).

A linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph). In this notation Ph means phenyl, (2,3 or 4)F-Ph means a phenyl groups substituted by F at either the 2-, 3- or 4-position.

A linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃).

A linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt).

A amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph. In this notation 2,(3,4,5 or 6)F₂-Ph means a phenyl group substituted by one F at the 2-position and a second F at either the 3, 4, 5, or 6 position. Where there are two substituents the may also be in the 3,(4 or 5) position if desired.

A cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl).

A cyclic C₃-C₈ alkyl group (such as cyclopropyl, cyclobutyl, cyclopentyl, cyclohexyl, cycloheptyl and cyclooctyl).

A linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH).

A linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH).

A linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂.

A linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe).

A linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt).

A linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph.

A linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe).

A linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂.

A sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃.

A sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe).

An aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃).

A cyclic aminosulphonyl- group (such as —N(SO₂)(CH₂)₃ and —N(SO₂)(CH₂)₄).

An aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph- , 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-).

A saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

In especially preferred embodiments R¹, R², R⁴, R⁵, and R⁸ are all H or R¹, R², R⁵, and R⁸ are all H and R⁴ is Me.

In typical embodiments each of R⁷¹, R⁷², R⁷³, R⁷⁴, R⁷⁵, and R⁷⁶ is independently H or a group selected from the following groups.

A halogen (such as F, Cl, Br and I).

A linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl).

A linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph). In this notation Ph means phenyl, (2,3 or 4)F-Ph means a phenyl groups substituted by F at either the 2-, 3- or 4-position.

A linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃).

A linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt).

A amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph. In this notation 2,(3,4,5 or 6)F₂-Ph means a phenyl group substituted by one F at the 2-position and a second F at either the 3, 4, 5, or 6 position. Where there are two substituents the may also be in the 3,(4 or 5) position if desired.

A cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl).

A cyclic C₃-C₅ alkyl group (such as cyclopropyl, cyclobutyl, cyclopentyl, cyclohexyl, cycloheptyl and cyclooctyl).

A linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH).

A linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH).

A linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂.

A linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe).

A linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt).

A linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph.

A linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe).

A linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂.

A sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃.

A sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe).

An aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃).

A cyclic aminosulphonyl- group (such as —N(SO₂)(CH₂)₃ and —N(SO₂)(CH₂)₄).

An aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph- , 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-).

A saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

In more typical embodiments each of R⁷¹ and R⁷² is H. Alternatively, two of R⁷¹ and/or two of R⁷ when attached to the same carbon atom may together form a ketone group. Typically R⁷³ and R⁷⁵ are not H.

In more preferred compounds, R⁷⁵ is a substituted or unsubstituted group selected from a linear or branched alkyl group, a cycloalkyl group, a saturated or unsaturated heterocyclic group, and an aryl group; chosen from the following groups.

A linear or branched C₁-C₆ alkyl group (such as Me, Et, Pr, i-Pr, n-Bu, i-Bu, t-Bu, pentyl and hexyl).

A linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph).

A linear or branched C₁-C₆ halogenated alkyl group (such as —CH₂F, —CH₂Cl, —CH₂Br, —CH₂I, —CF₃, —CCl₃, —CBr₃, and —Cl₃).

A linear or branched primary secondary or tertiary C₁-C₆ alkylamine group (such as —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt).

A cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);

A cyclic C₃-C₅ alkyl group (such as cyclopropyl, cyclobutyl, cyclopentyl, cyclohexyl, cycloheptyl and cyclooctyl).

A linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH).

A linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH).

A linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe).

A linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt).

A linear or branched C₁-C₇ alkoxyalkyl or aryloxyalkyl group (such as CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe).

An aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph- , 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-).

A saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

In some embodiments, two of R⁷¹ and/or two of R⁷² when attached to the same carbon atom may together form a ketone group.

In the present case, especially preferred compounds of the invention are those of the following formulae:

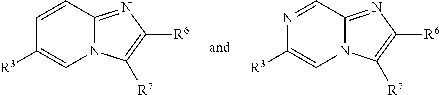

wherein R³, R⁶ and R⁷ are as defined below:

In these especially preferred embodiments R³ may be any substituent as defined above, but is preferably selected from the following:

An unsubstituted, 2-monosubstituted, or 2,6-disubstituted phenyl group, preferably where the substituent is selected from F, Cl and CN (such as Ph-, 2-F-Ph-, 2,6-F₂-Ph-, 2-Cl-Ph-, 2,6-Cl₂-Ph-, 2-CN-Ph-, 2,6-(CN)₂-Ph-, 2,6-F, CN-Ph-, 2,6-F, Cl-Ph-, and 2,6-Cl, CN-Ph-).

An unsubstituted or 3-monosubstituted pyridine-2-yl, or a 2,6-disubstituted pyridine-4-yl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂—Pyr-4-yl. In this context Pyr means pyridine.

An unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl).

A substituted or unsubstituted 1,2,4-oxadiazol-3-yl group.

A cyclic aminosulphonyl- group (such as —N(SO₂)(CH₂)₃ and —N(SO₂)(CH₂)₄).

A linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe.

A linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl).

A linear or branched sulphonyl group (such as —SO₂Me, —SO₂Et, —SO₂Pr, —SO₂iPr).

A thioether group (such as —SMe, —SEt, —SPr, and SiPr).

A branched or cyclic alkyl or alkenyl group having 3 carbon atoms or more, preferably having from 3 to 6 carbon atoms, and preferably selected from an isopropyl, a cyclopropyl and a propen-2-yl group.

In these especially preferred embodiments R⁶ may be any substituent as defined above, but is preferably selected from the following:

An unsubstituted, 2-, 3- or 4-monosubstituted, and a 2,4 or 3,4-disubstituted phenyl group, preferably wherein the substituent is F, Cl or —OMe (such as 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph).

A substituted or unsubstituted pyridine-2-yl, pyridine-3-yl or pyridine-4-yl group preferably wherein the substituent is F or Cl (such as pyridine-2-yl, pyridine-3-yl, pyridine-4-yl, 3-F-pyridine-2-yl, 3-Cl-pyridine-2-yl, 4-F-pyridine-2-yl, 4-Cl-pyridine-2-yl, 5-F-pyridine-2-yl, 5-Cl-pyridine-2-yl.

A cyclic ether group (such as tetrahydrofuran-2-yl, tetrahydrofuran-3-yl, terahydropyran-2-yl, tetrahydropyran-3-yl and tetrahydropyran-4-yl).

In these especially preferred embodiments R⁷ may be any substituent as defined above, but is preferably selected from the following:

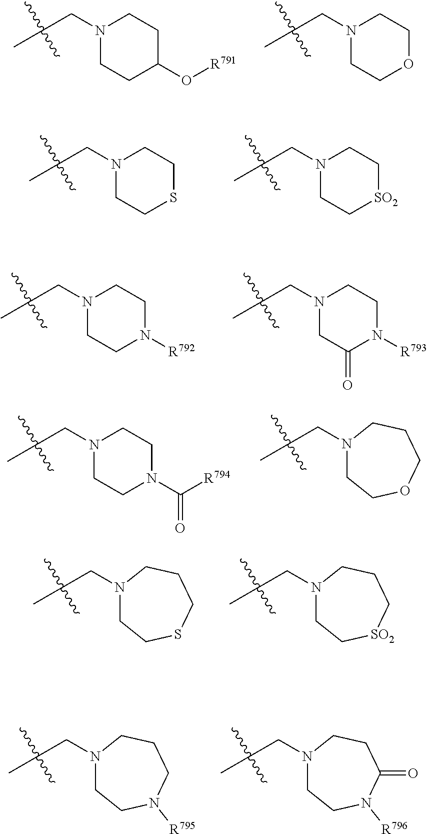

and also from a group having the following structure:

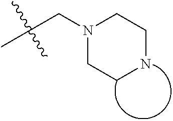 [SMILES: *CN1CCNCC1]

where the ring fused to the piperazine ring is a 5- or 6-membered ring optionally having 1 or more further heteroatoms in the ring selected from N and O and optionally having one or more substituents, preferably wherein this group is selected from the following:

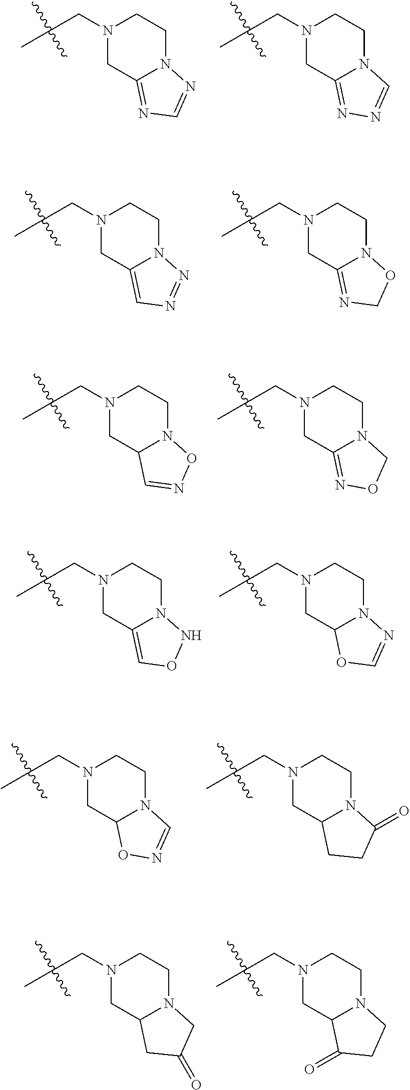

wherein R⁷⁹¹, R⁷⁹², R⁷⁹³, R⁷⁹⁴. R⁷⁹⁵, and R⁷⁹⁶ are selected from the following:

R⁷⁹¹ is selected from H, Me, Et, Pr, iPr, cyPr, and cyBu, preferably H.

R⁷⁹² is selected from H, Me, Et, Pr, iPr, cyPr, cyBu, —(CO)-Me, —(CO)-Et, —(CO)—Pr, —(CO)iPr, —SO₂Me, and SO₂Et, preferably H, Me, —SO₂Me and —(CO)-Me.

R⁷⁹³ is selected from H, Me, Et, Pr, iPr, cyPr, cyBu, —(CO)-Me, —(CO)Et, —(CO)Pr, and —(CO)iPr, preferably H, Me and —(CO)-Me.

R⁷⁹⁴ is selected from H, Me, Et, Pr, iPr, cyPr, cyBu; an unsubstituted, 2-, 3- or 4-monosubstituted, and a 2,4 or 3,4-disubstituted phenyl group, preferably wherein the substituent is F, ClCN, or —OMe (such as 2-F-Ph, 2-Cl-Ph, 2-CN-Ph, 2-OMe-Ph, 3-F-Ph, 3-Cl-Ph, 3-CN-Ph, 3-OMe-Ph, 4-F-Ph, 4-Cl-Ph, 4-CN-Ph, 4-OMe-Ph, 2,4-F₂-Ph, 2,4-Cl₂-Ph, 2,4-(CN)₂-Ph, 2,4-(OMe)₂-Ph, 3,4-F₂-Ph, 3,4-Cl₂-Ph, 3,4-(CN)₂-Ph, and 3,4-(OMe)₂-Ph); —NH₂, —NHMe, and —NMe₂; —OH, —OMe, —OEt, —OPr, —OiPr, —OⁿBu, and —O^{t}Bu; —CH₂OH, —CHMeOH, —C(Me)₂OH, —CH₂OMe, —CHMeOMe, —C(Me)₂OMe, —CH₂OEt, —CHMeOEt, and —C(Me)₂OEt; and a heterocyclic ring group having from 4-7 ring atoms with at least one heteroatom selected from N, O, and S, which ring group may be saturated or unsaturated and may be substituted or unsubstituted, preferably wherein the heterocyclic ring group is selected from the following groups:

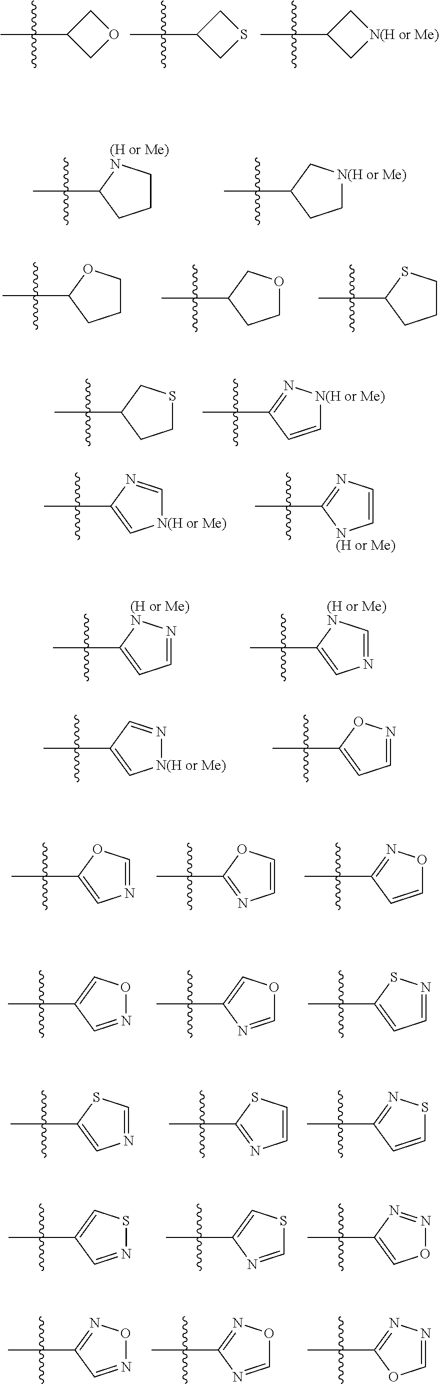

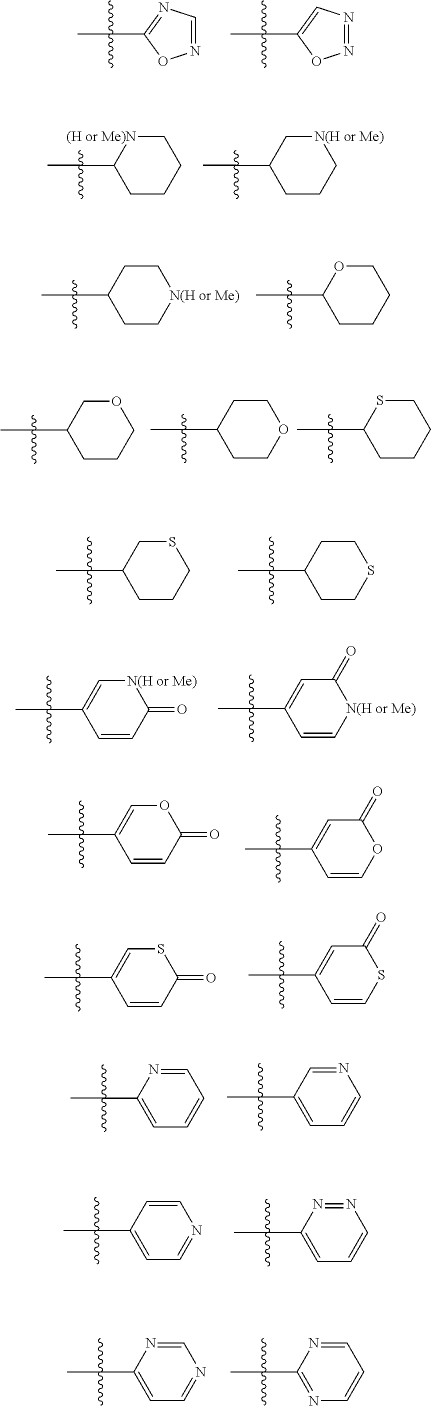

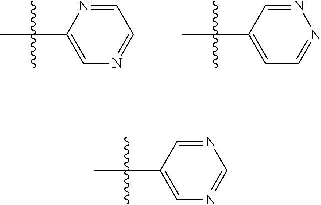 [SMILES: *c1cnccn1 ; *c1ccnnc1 ; *c1cncnc1]

R⁷⁹⁵ is selected from H, Me, Et, Pr, iPr, cyPr, cyBu, —(CO)-Me, —(CO)-Et, —(CO)—Pr, —(CO)iPr, —SO₂Me, and SO₂Et, preferably H, Me, —SO₂Me and —(CO)-Me.

R⁷⁹⁶ is selected from H, Me, Et, Pr, iPr, cyPr, cyBu, —(CO)-Me, —(CO)Et, —(CO)Pr, and —(CO)iPr, preferably H, Me and —(CO)-Me.

In these especially preferred embodiments R¹, R², and R⁴ may be any substituent as defined above, but are preferably all H.

Within these especially preferred compounds, those of the following structures (A1), (A2), (A3), and (A4) are most preferred:

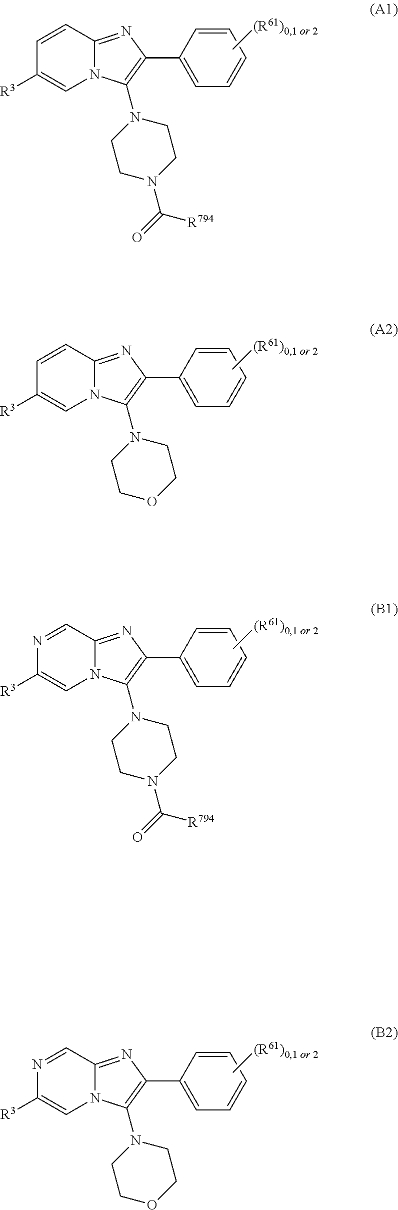

In these structures R³ and R⁷⁹⁴ may be any of the groups as already defined above, but preferably are as follows:

R³ is preferably selected from the following:

An unsubstituted, 2-monosubstituted, or 2,6-disubstituted phenyl group, preferably where the substituent is selected from F, Cl and CN (such as Ph-, 2-F-Ph-, 2,6-F₂-Ph-, 2-Cl-Ph-, 2,6-Cl₂-Ph-, 2-CN-Ph-, 2,6-(CN)₂-Ph-, 2,6-F, CN-Ph-, 2,6-F, Cl-Ph-, and 2,6-Cl, CN-Ph-).

An unsubstituted or 3-monosubstituted pyridine-2-yl, or a 2,6-disubstituted pyridine-4-yl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂—Pyr-4-yl. In this context Pyr means pyridine.

An unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl).

A substituted or unsubstituted 1,2,4-oxadiazol-3-yl group.

A cyclic aminosulphonyl- group (such as —N(SO₂)(CH₂)₃ and —N(SO₂)CH₂)₄).

A linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe.

A linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl).

A linear or branched sulphonyl group (such as —SO₂Me, —SO₂Et, —SO₂Pr, —SO₂iPr).

A thioether group (such as —SMe, —SEt, —SPr, and SiPr).

An isopropyl, cyclopropyl and a propen-2-yl group.

In compounds of type (B1) and (B2), the R³ group is particularly preferably selected from an isopropyl, cyclopropyl and a propen-2-yl group.

R⁷⁹⁴ is preferably selected from the following: H, Me, Et, Pr, iPr, cyPr, cyBu; an unsubstituted, 2-, 3- or 4-monosubstituted, and a 2,4 or 3,4-disubstituted phenyl group, preferably wherein the substituent is F, ClCN, or —OMe (such as 2-F-Ph, 2-Cl-Ph, 2-CN-Ph, 2-OMe-Ph, 3-F-Ph, 3-Cl-Ph, 3-CN-Ph, 3-OMe-Ph, 4-F-Ph, 4-Cl-Ph, 4-CN-Ph, 4-OMe-Ph, 2,4-F₂-Ph, 2,4-Cl₂-Ph, 2,4-(CN)₂-Ph, 2,4-(OMe)₂-Ph, 3,4-F₂-Ph, 3,4-Cl₂-Ph, 3,4-(CN)₂-Ph, and 3,4-(OMe)₂-Ph); —NH₂, —NHMe, and —NMe₂; —OH, —OMe, —OEt, —OPr, —OiPr, —OⁿBu, and —O^{t}Bu; —CH₂OH, —CHMeOH, —C(Me)₂OH, —CH₂OMe, —CHMeOMe, —C(Me)₂OMe, —CH₂OEt, —CHMeOEt, and —C(Me)₂OEt; and a heterocyclic ring group having from 4-7 ring atoms with at least one heteroatom selected from N, O, and S, which ring group may be saturated or unsaturated and may be substituted or unsubstituted, preferably wherein the heterocyclic ring group is selected from the following groups:

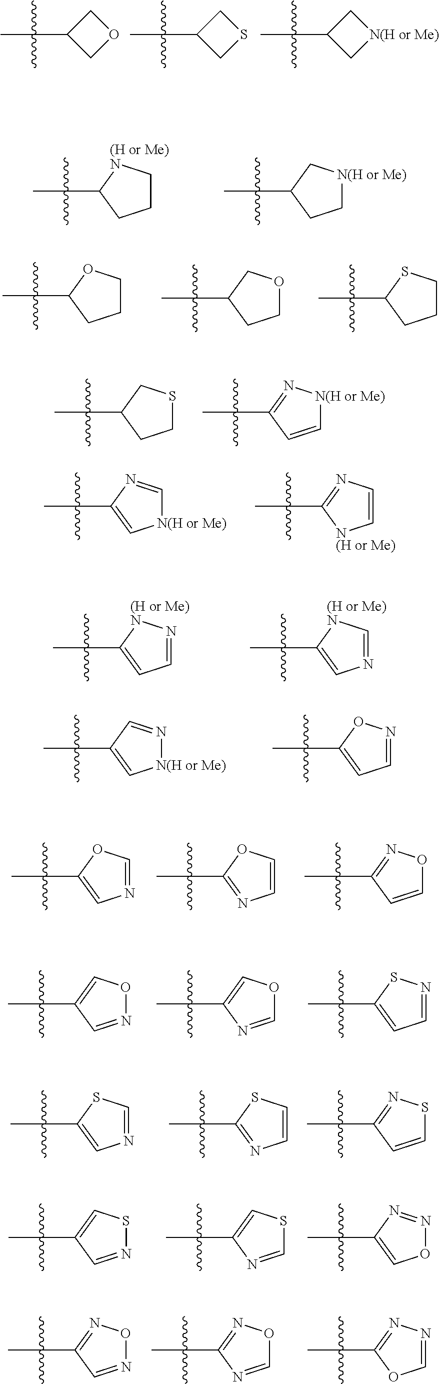

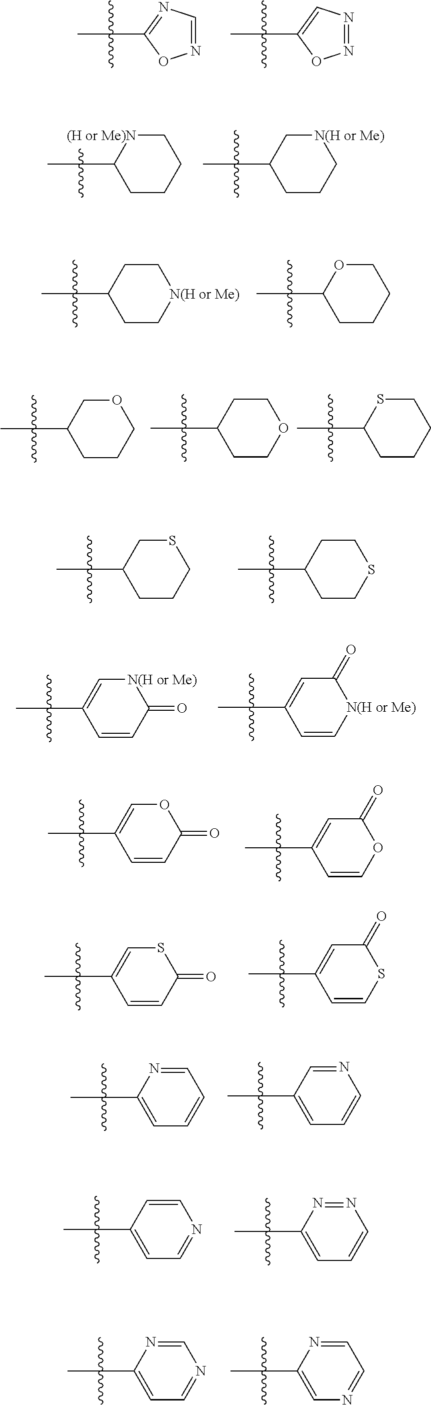

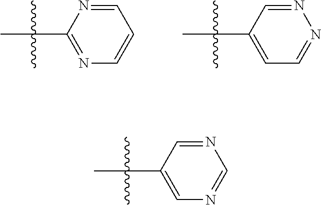 [SMILES: *c1ncccn1 ; *c1ccnnc1 ; *c1cncnc1]

when present, R⁶¹ is a substituent (or two substituents which may be the same or different) on the phenyl ring and may be selected from F, Cl, and —OMe. Thus the phenyl ring may be unsubstituted, 2-, 3- or 4-monosubstituted, or 2,4 or 3,4-disubstituted, such that the -Ph-R⁶¹_{(0,1, or 2)} group as a whole is preferably selected from 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph)

Thus, with regard to the above, the present invention provides the following SLC2A class I transporter inhibitor compounds, typically GLUT1, GLUT2, GLUT3, GLUT4 and/or GLUT14 inhibitor compounds, for use in medicine:

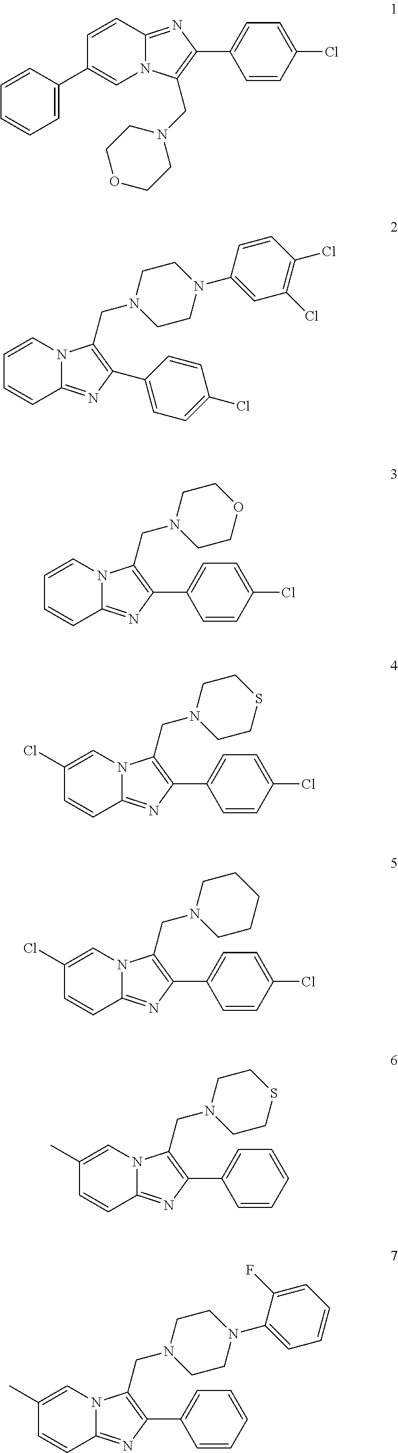

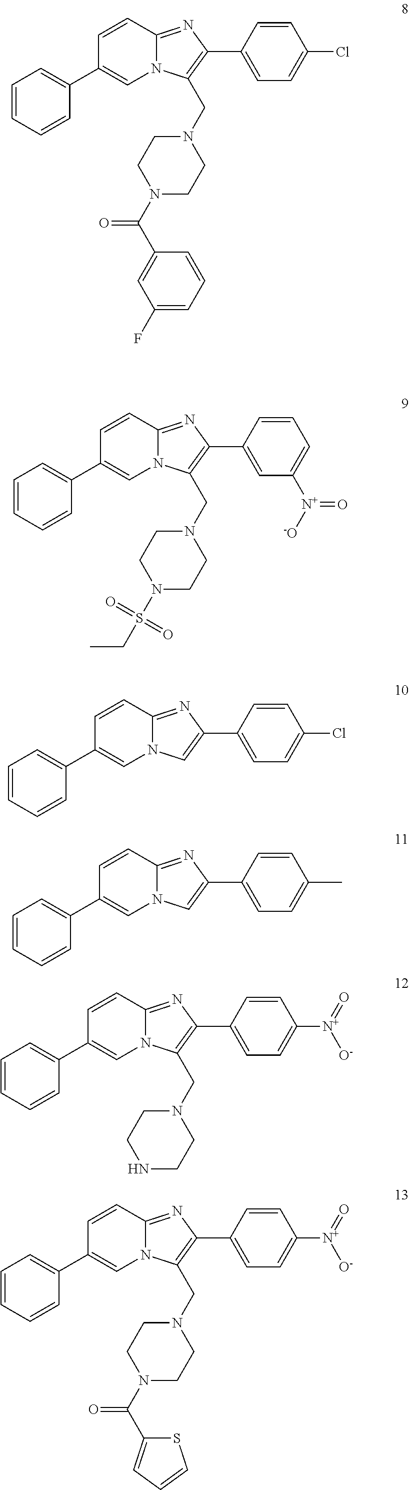

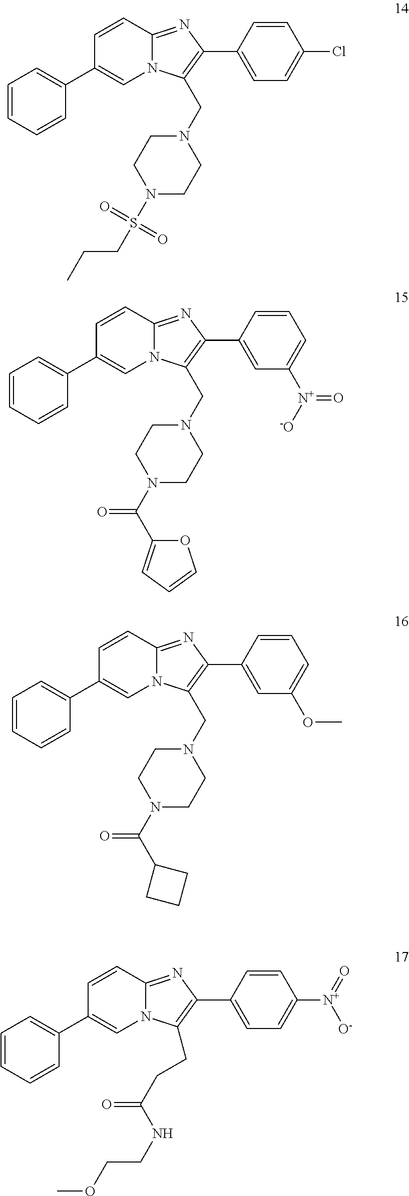

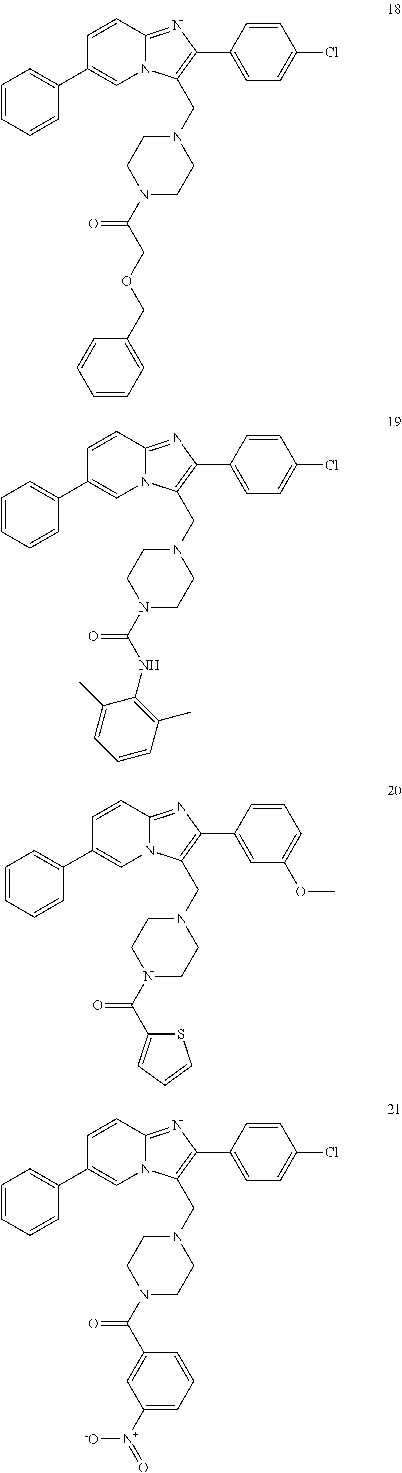

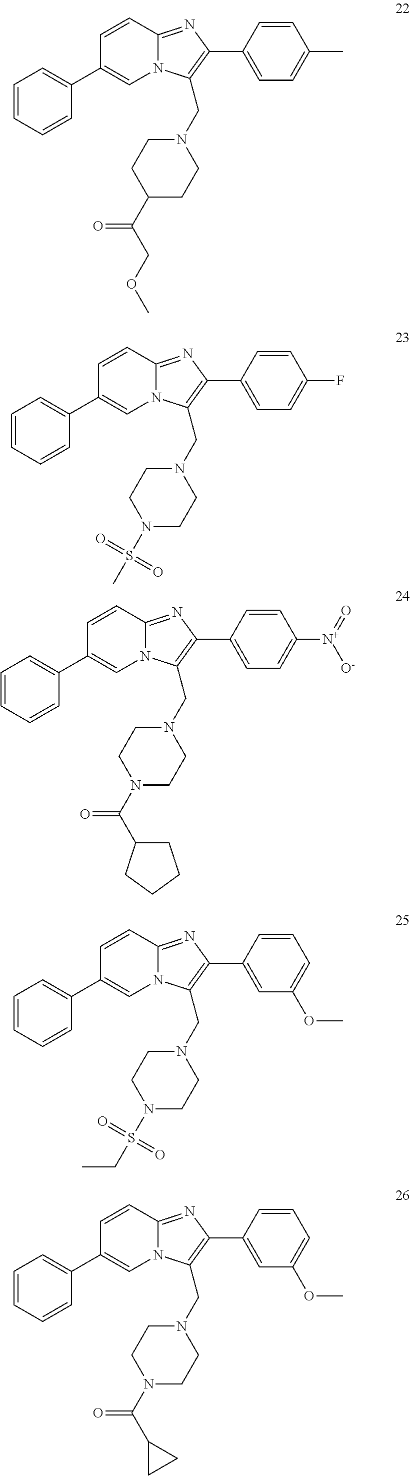

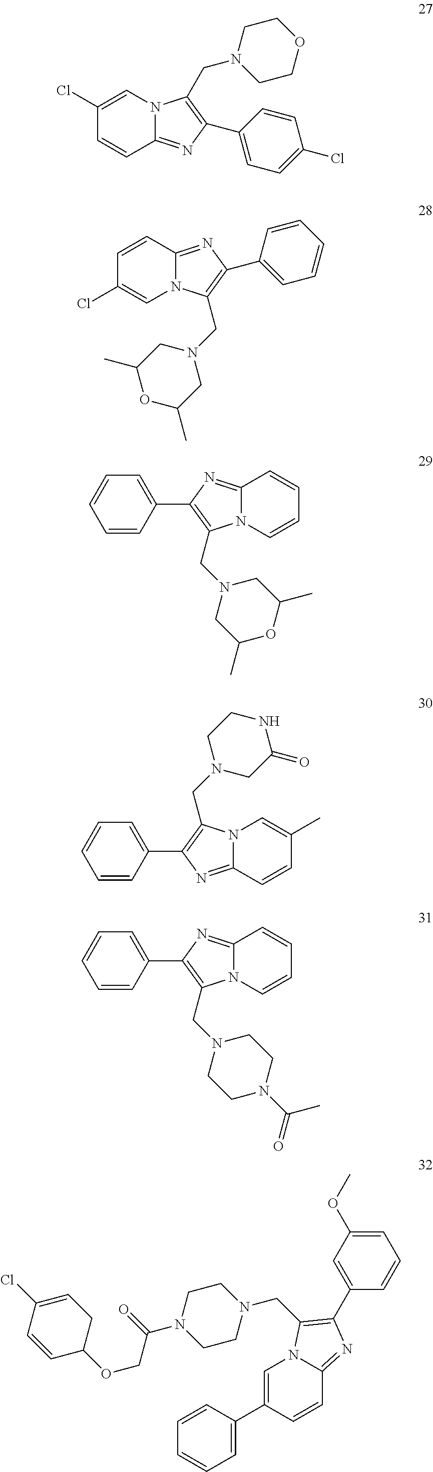

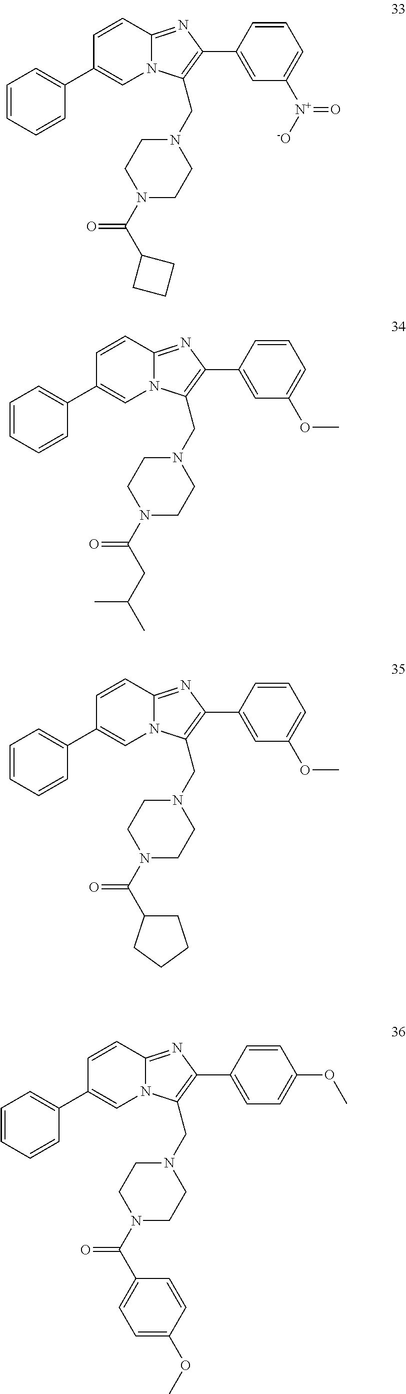

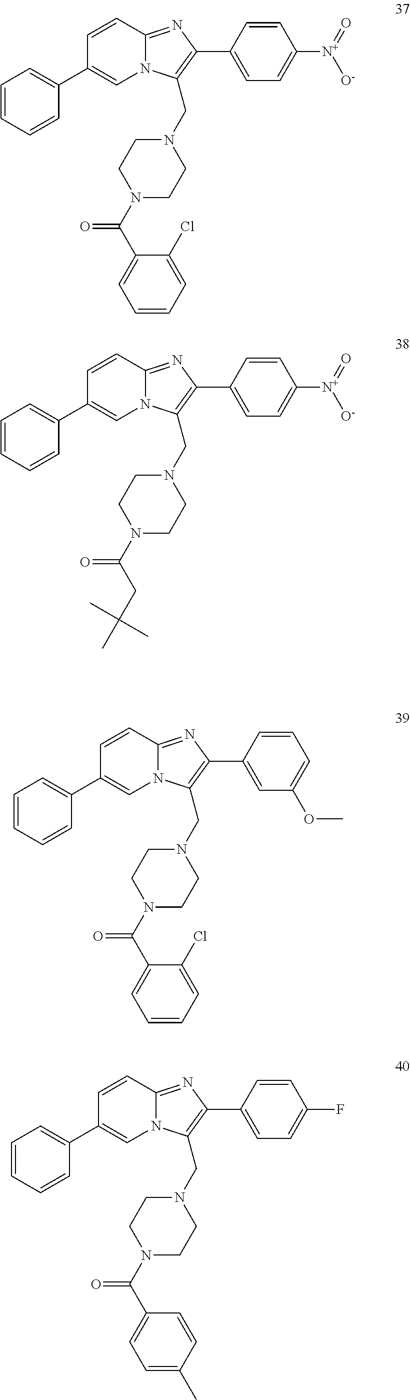

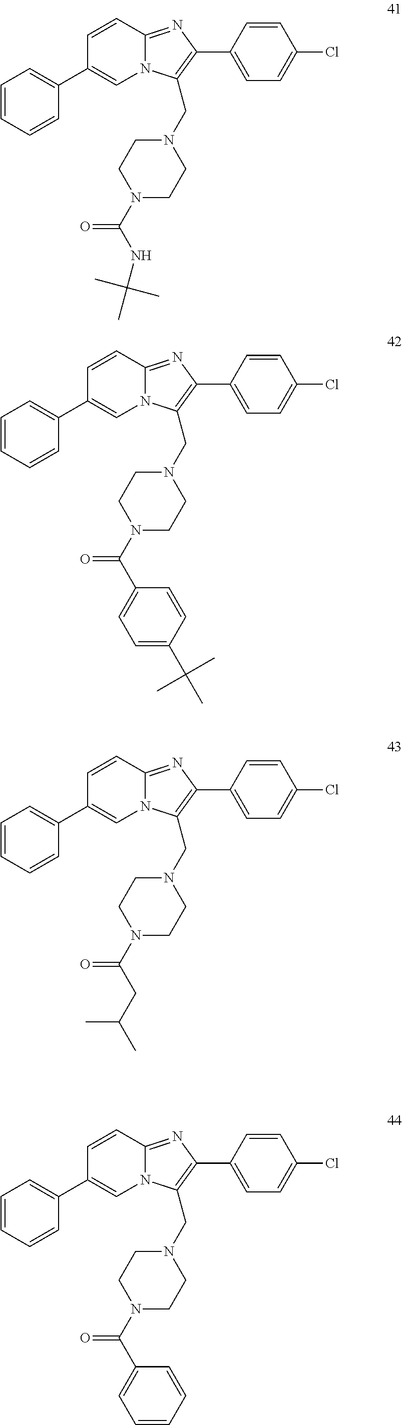

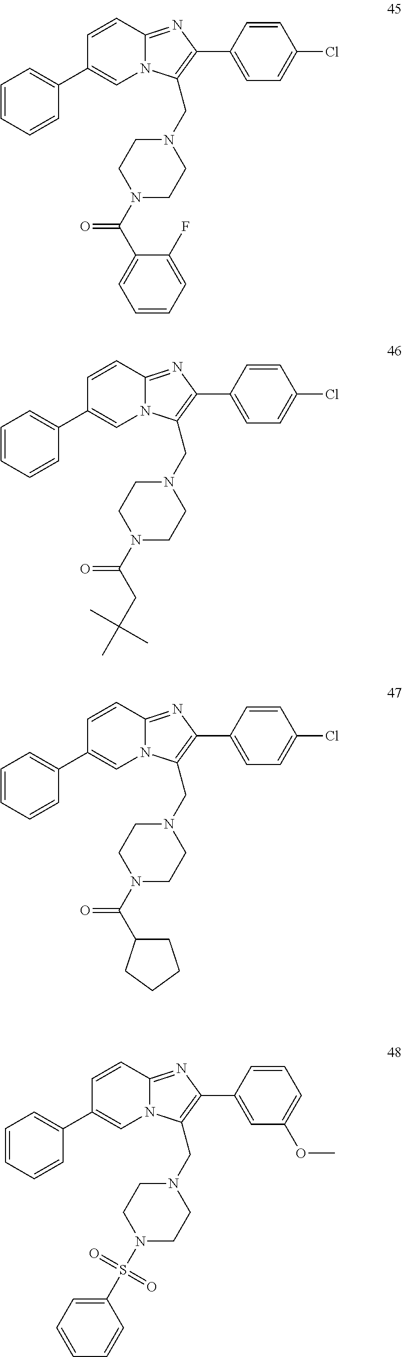 [SMILES: O=C(c1ccccc1F)N1CCN(Cc2c(-c3ccc(Cl)cc3)nc3ccc(-c4ccccc4)cn23)CC1 ; CC(C)(C)CC(=O)N1CCN(Cc2c(-c3ccc(Cl)cc3)nc3ccc(-c4ccccc4)cn23)CC1 ; O=C(C1CCCC1)N1CCN(Cc2c(-c3ccc(Cl)cc3)nc3ccc(-c4ccccc4)cn23)CC1 ; COc1cccc(-c2nc3ccc(-c4ccccc4)cn3c2CN2CCN(S(=O)(=O)c3ccccc3)CC2)c1]

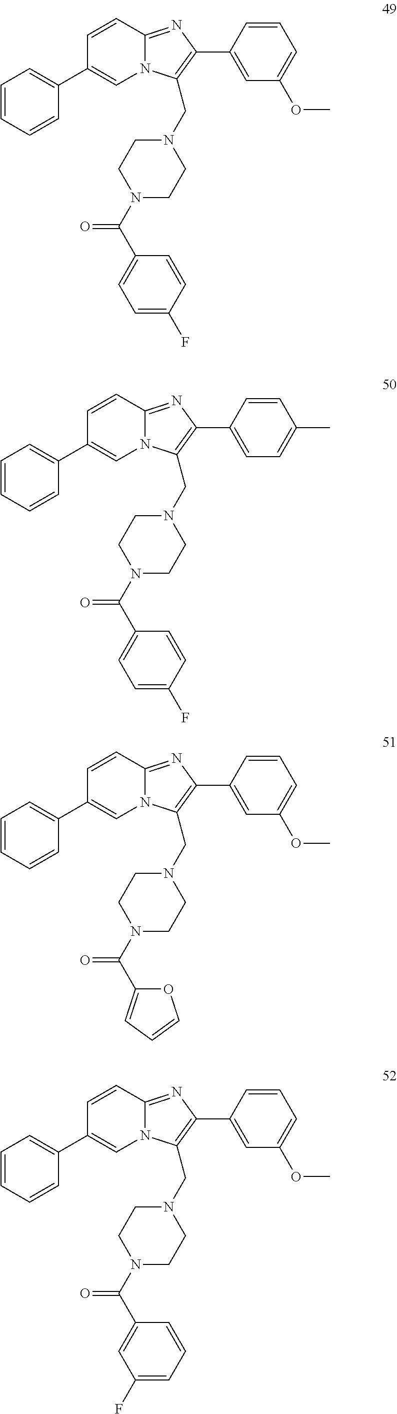

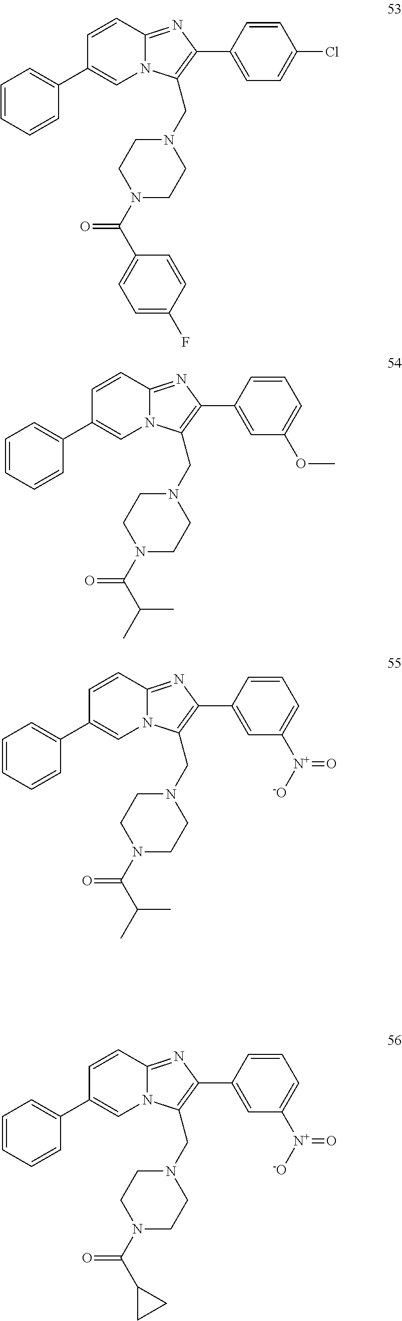

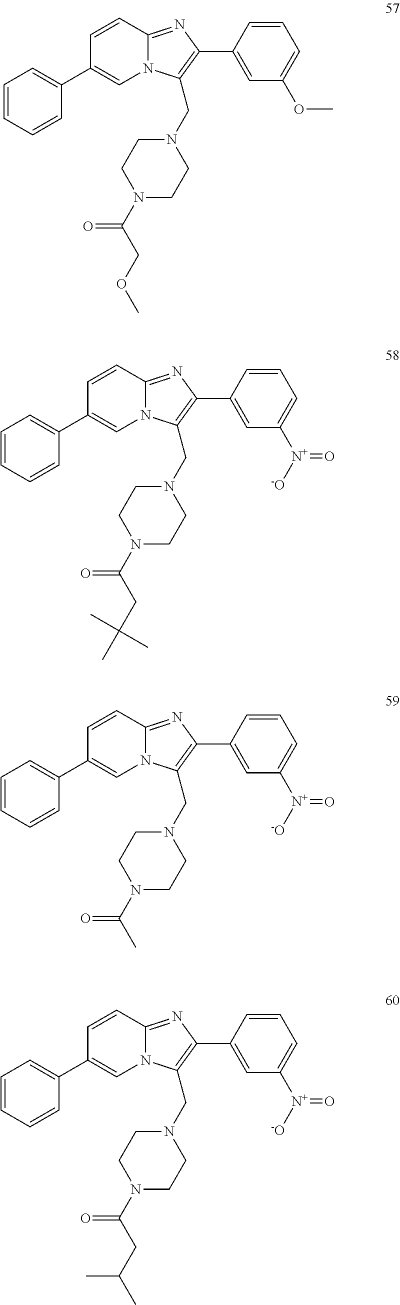

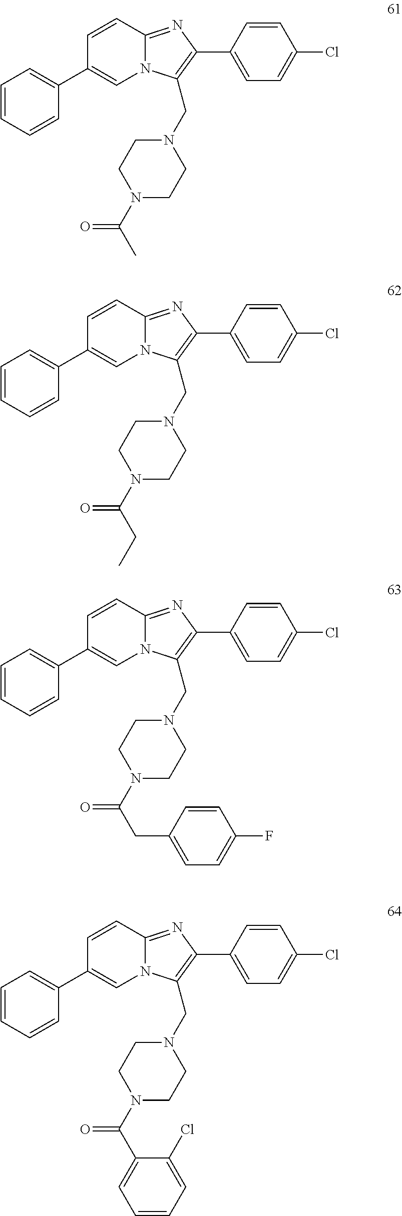

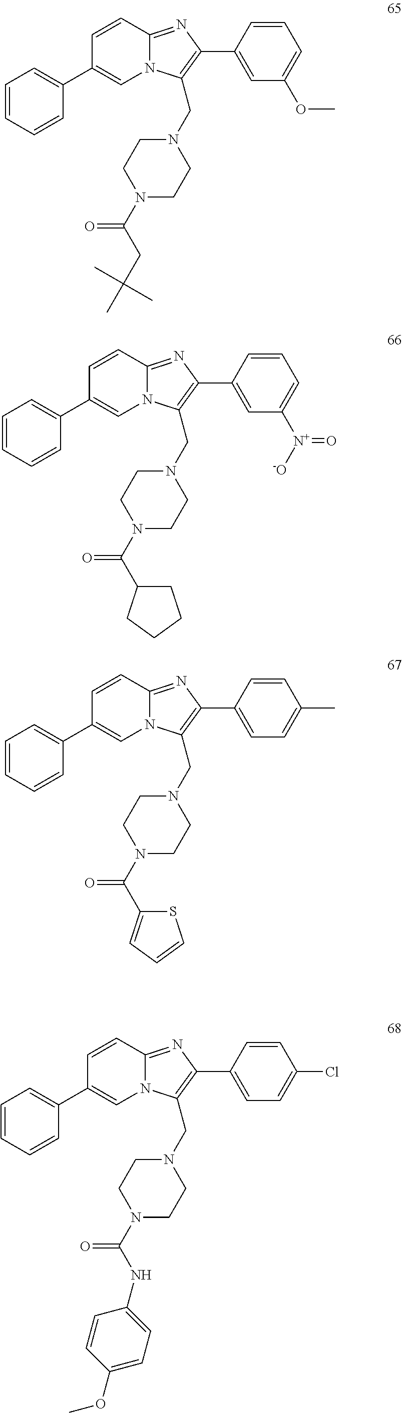

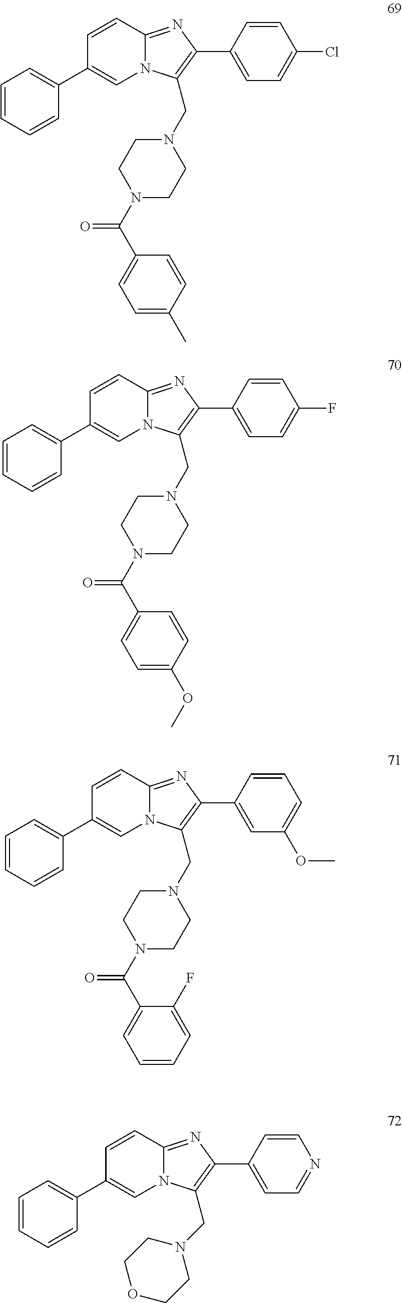

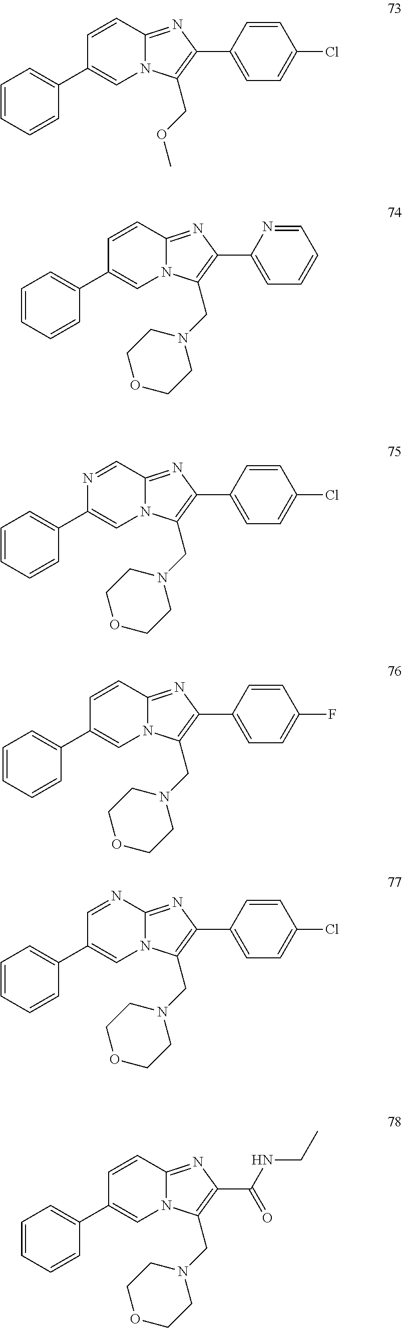 [SMILES: COCc1c(-c2ccc(Cl)cc2)nc2ccc(-c3ccccc3)cn12 ; c1ccc(-c2ccc3nc(-c4ccccn4)c(CN4CCOCC4)n3c2)cc1 ; Clc1ccc(-c2nc3cnc(-c4ccccc4)cn3c2CN2CCOCC2)cc1 ; Fc1ccc(-c2nc3ccc(-c4ccccc4)cn3c2CN2CCOCC2)cc1 ; Clc1ccc(-c2nc3ncc(-c4ccccc4)cn3c2CN2CCOCC2)cc1 ; CCNC(=O)c1nc2ccc(-c3ccccc3)cn2c1CN1CCOCC1]

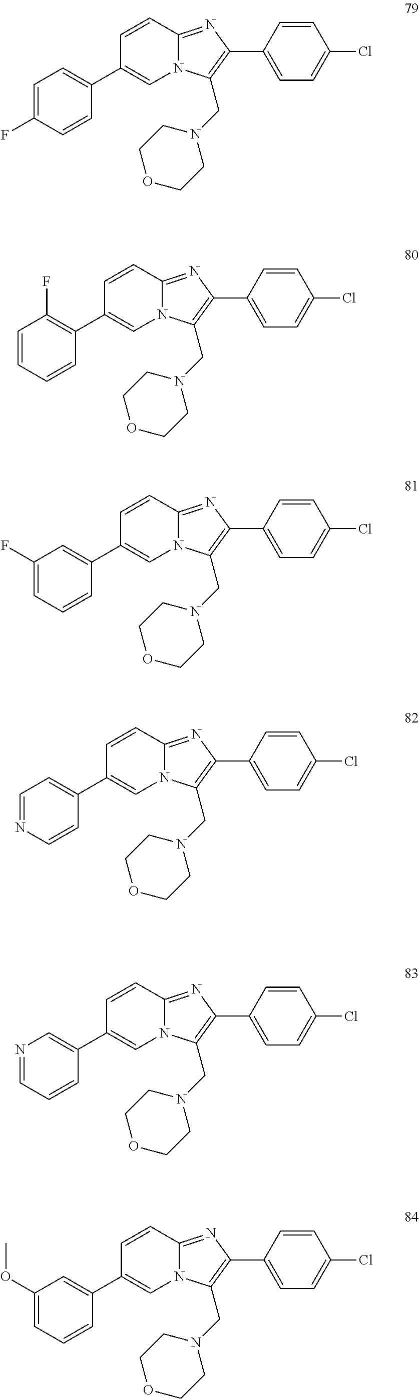 [SMILES: Fc1ccc(-c2ccc3nc(-c4ccc(Cl)cc4)c(CN4CCOCC4)n3c2)cc1 ; Fc1ccccc1-c1ccc2nc(-c3ccc(Cl)cc3)c(CN3CCOCC3)n2c1 ; Fc1cccc(-c2ccc3nc(-c4ccc(Cl)cc4)c(CN4CCOCC4)n3c2)c1 ; Clc1ccc(-c2nc3ccc(-c4ccncc4)cn3c2CN2CCOCC2)cc1 ; Clc1ccc(-c2nc3ccc(-c4cccnc4)cn3c2CN2CCOCC2)cc1 ; COc1cccc(-c2ccc3nc(-c4ccc(Cl)cc4)c(CN4CCOCC4)n3c2)c1]

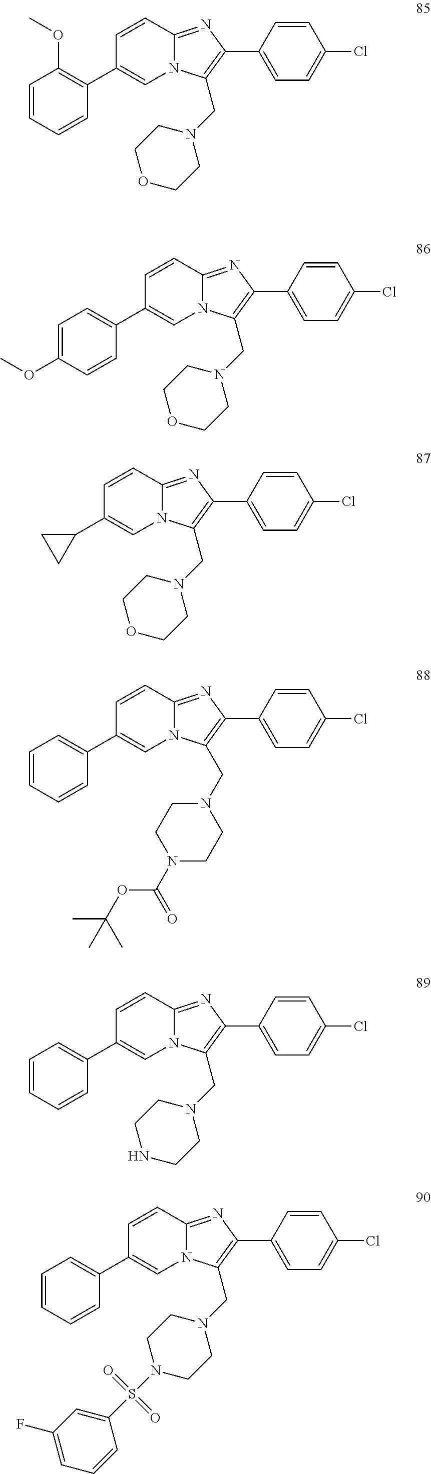

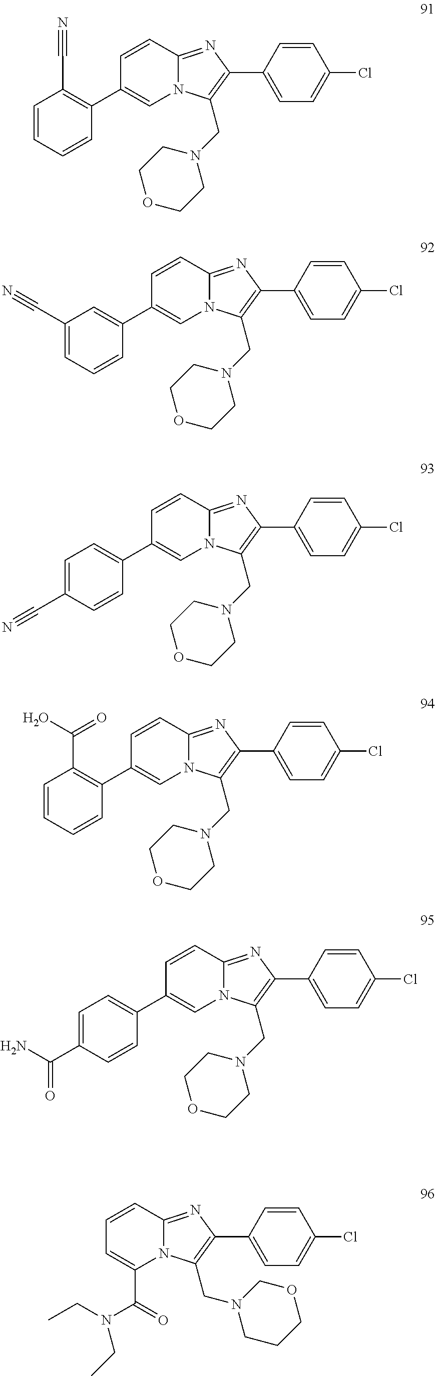

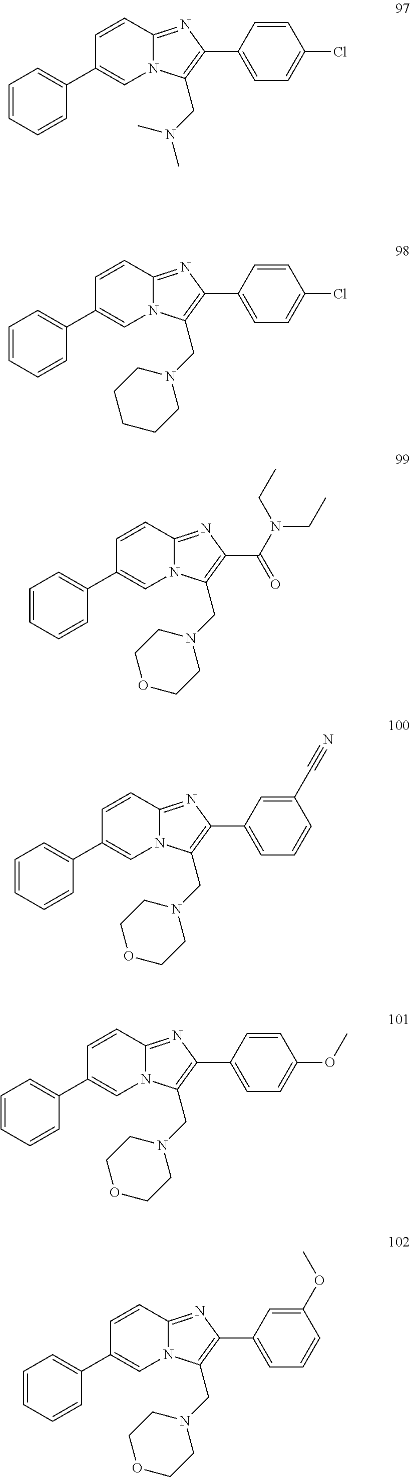 [SMILES: CN(C)Cc1c(-c2ccc(Cl)cc2)nc2ccc(-c3ccccc3)cn12 ; Clc1ccc(-c2nc3ccc(-c4ccccc4)cn3c2CN2CCCCC2)cc1 ; CCN(CC)C(=O)c1nc2ccc(-c3ccccc3)cn2c1CN1CCOCC1 ; N#Cc1cccc(-c2nc3ccc(-c4ccccc4)cn3c2CN2CCOCC2)c1 ; COc1ccc(-c2nc3ccc(-c4ccccc4)cn3c2CN2CCOCC2)cc1 ; COc1cccc(-c2nc3ccc(-c4ccccc4)cn3c2CN2CCOCC2)c1]

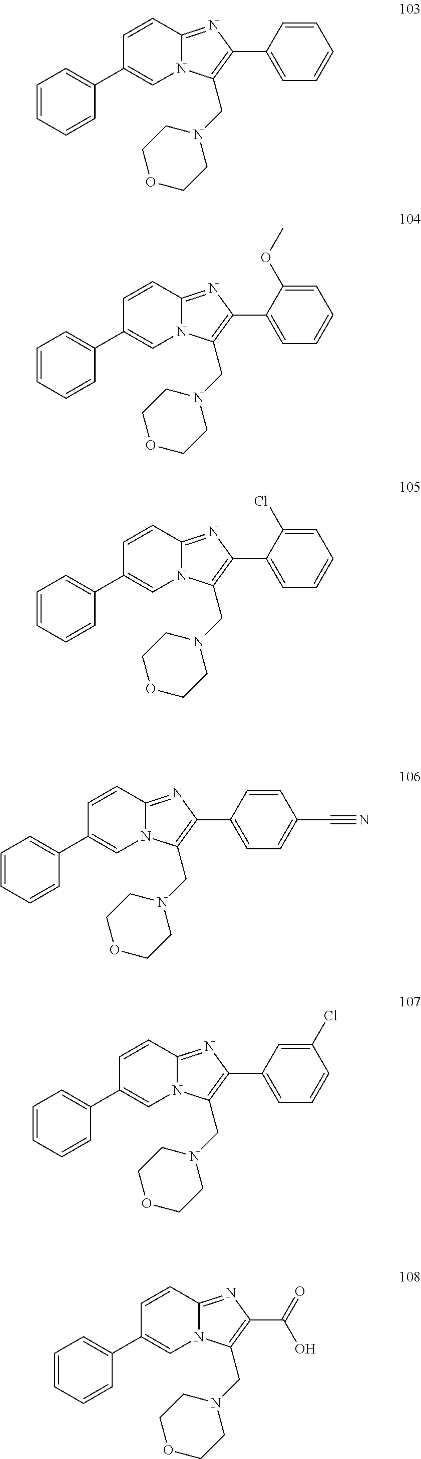 [SMILES: c1ccc(-c2ccc3nc(-c4ccccc4)c(CN4CCOCC4)n3c2)cc1 ; COc1ccccc1-c1nc2ccc(-c3ccccc3)cn2c1CN1CCOCC1 ; Clc1ccccc1-c1nc2ccc(-c3ccccc3)cn2c1CN1CCOCC1 ; N#Cc1ccc(-c2nc3ccc(-c4ccccc4)cn3c2CN2CCOCC2)cc1 ; Clc1cccc(-c2nc3ccc(-c4ccccc4)cn3c2CN2CCOCC2)c1 ; O=C(O)c1nc2ccc(-c3ccccc3)cn2c1CN1CCOCC1]

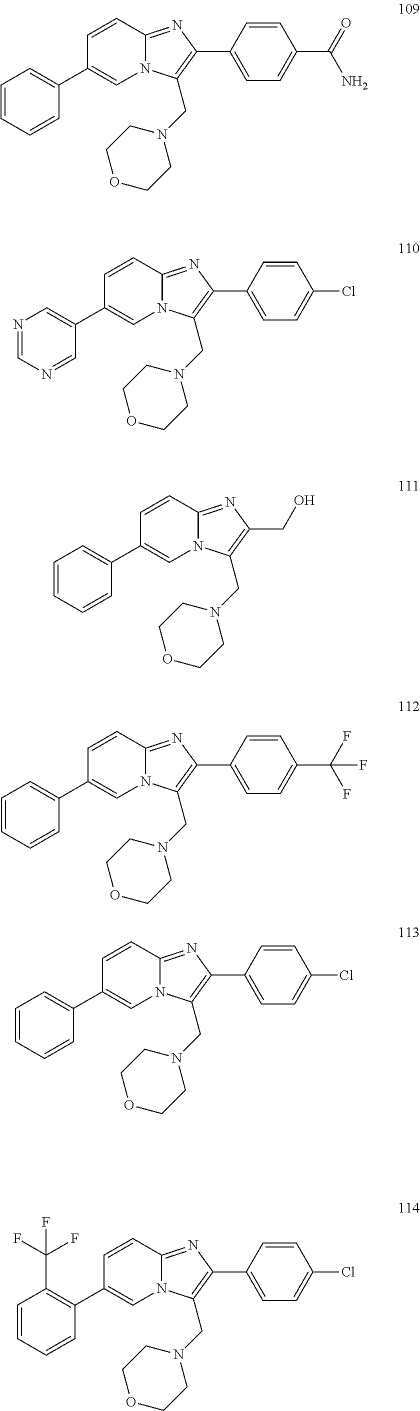

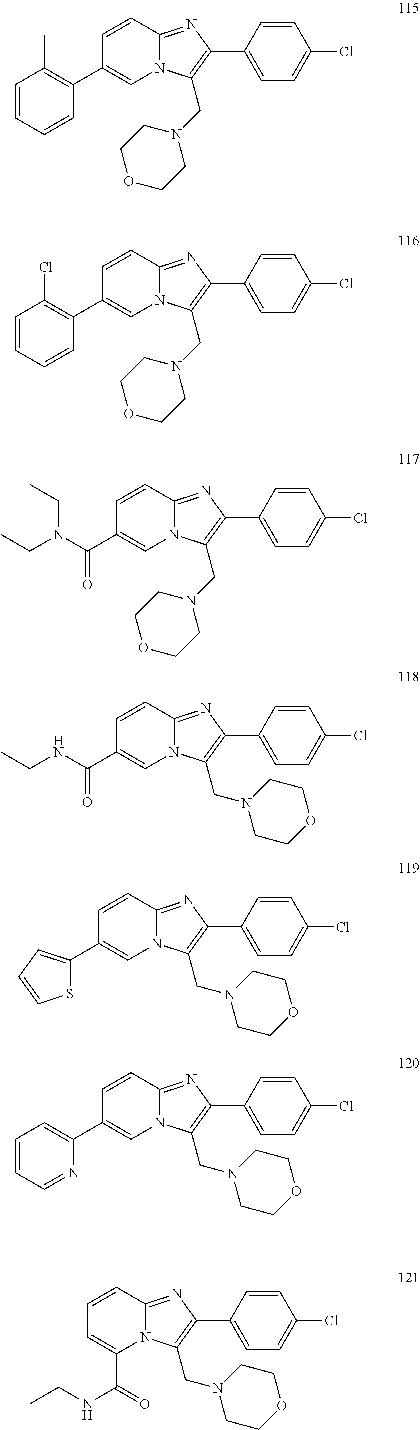

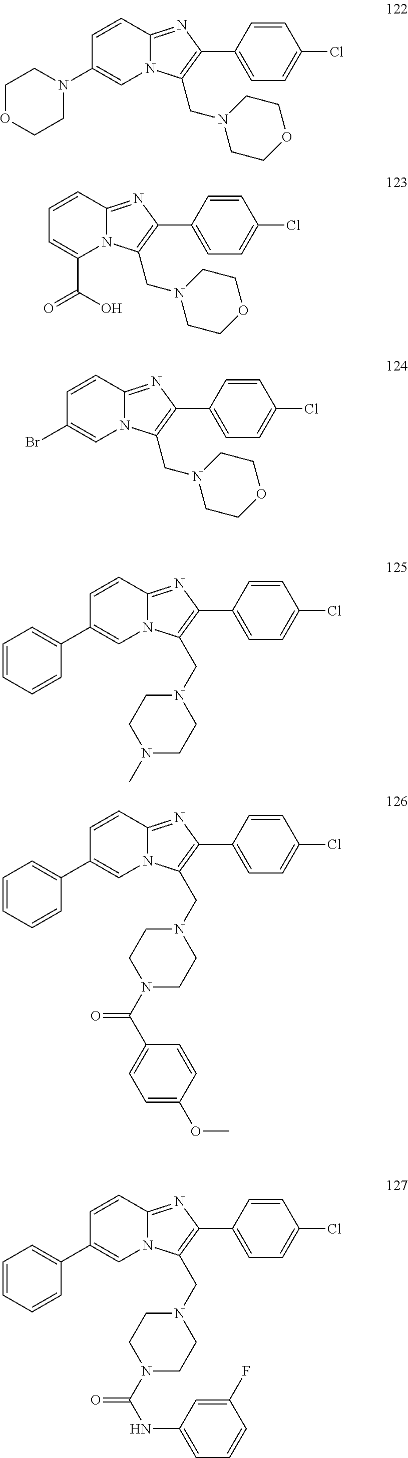

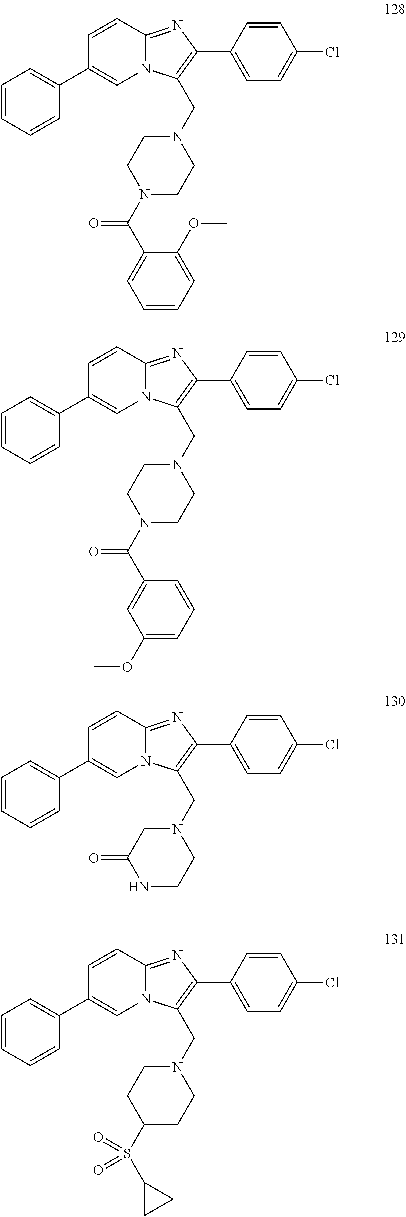

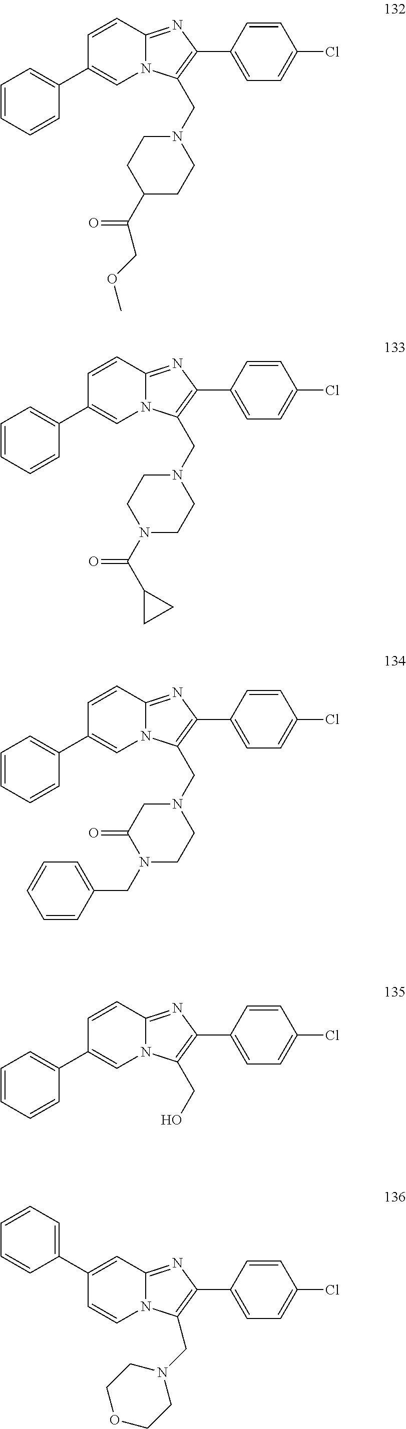

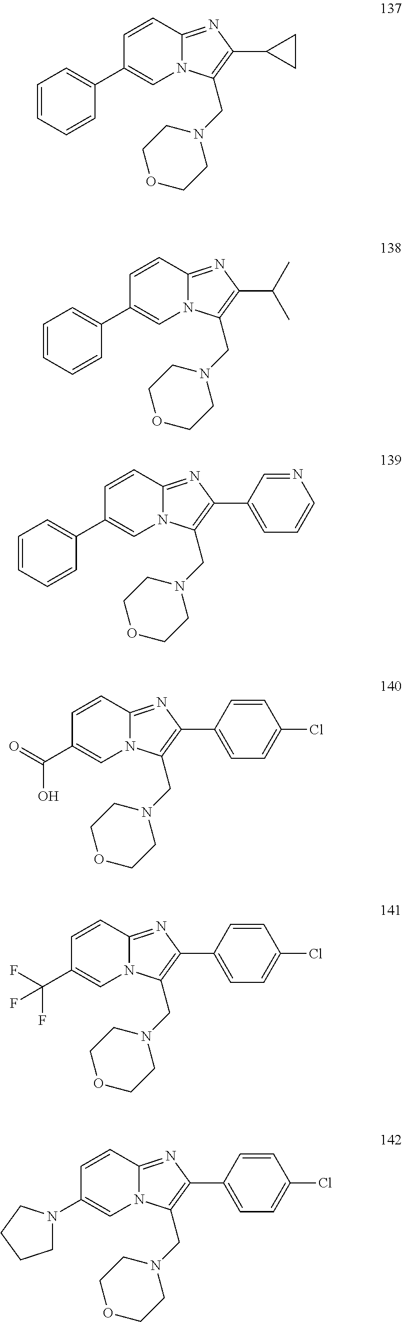 [SMILES: c1ccc(-c2ccc3nc(C4CC4)c(CN4CCOCC4)n3c2)cc1 ; CC(C)c1nc2ccc(-c3ccccc3)cn2c1CN1CCOCC1 ; c1ccc(-c2ccc3nc(-c4cccnc4)c(CN4CCOCC4)n3c2)cc1 ; O=C(O)c1ccc2nc(-c3ccc(Cl)cc3)c(CN3CCOCC3)n2c1 ; FC(F)(F)c1ccc2nc(-c3ccc(Cl)cc3)c(CN3CCOCC3)n2c1 ; Clc1ccc(-c2nc3ccc(N4CCCC4)cn3c2CN2CCOCC2)cc1]

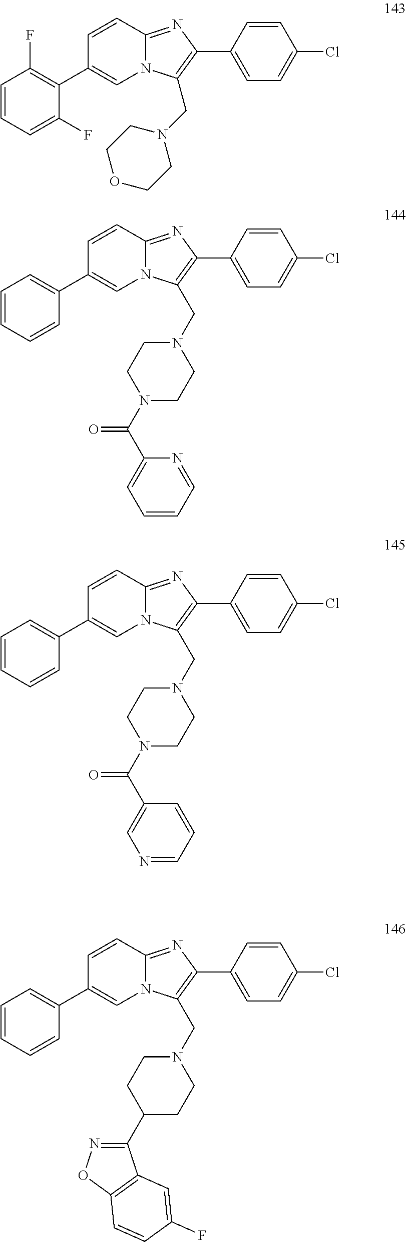

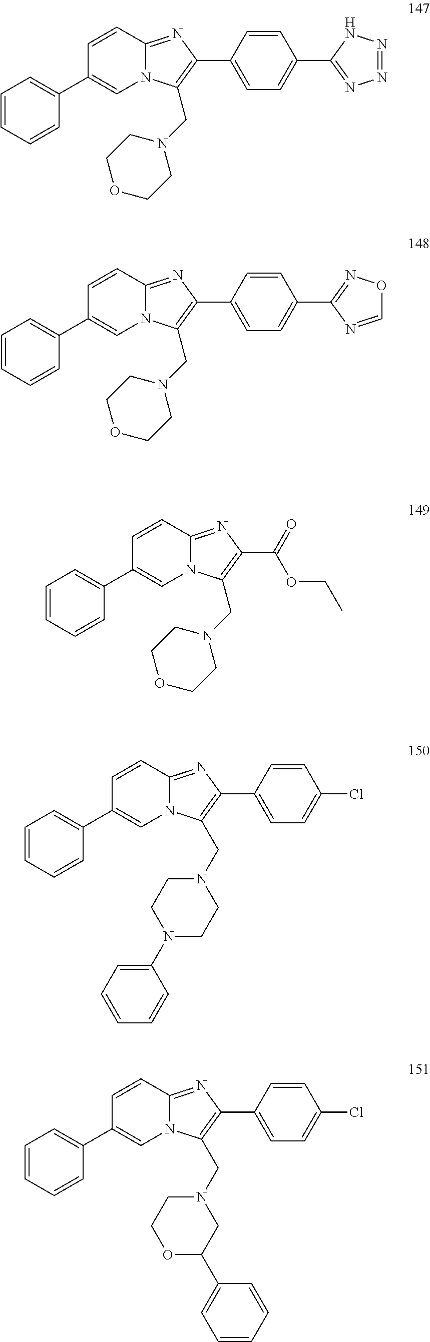 [SMILES: c1ccc(-c2ccc3nc(-c4ccc(-c5nnn[nH]5)cc4)c(CN4CCOCC4)n3c2)cc1 ; c1ccc(-c2ccc3nc(-c4ccc(-c5ncon5)cc4)c(CN4CCOCC4)n3c2)cc1 ; CCOC(=O)c1nc2ccc(-c3ccccc3)cn2c1CN1CCOCC1 ; Clc1ccc(-c2nc3ccc(-c4ccccc4)cn3c2CN2CCN(c3ccccc3)CC2)cc1 ; Clc1ccc(-c2nc3ccc(-c4ccccc4)cn3c2CN2CCOC(c3ccccc3)C2)cc1]

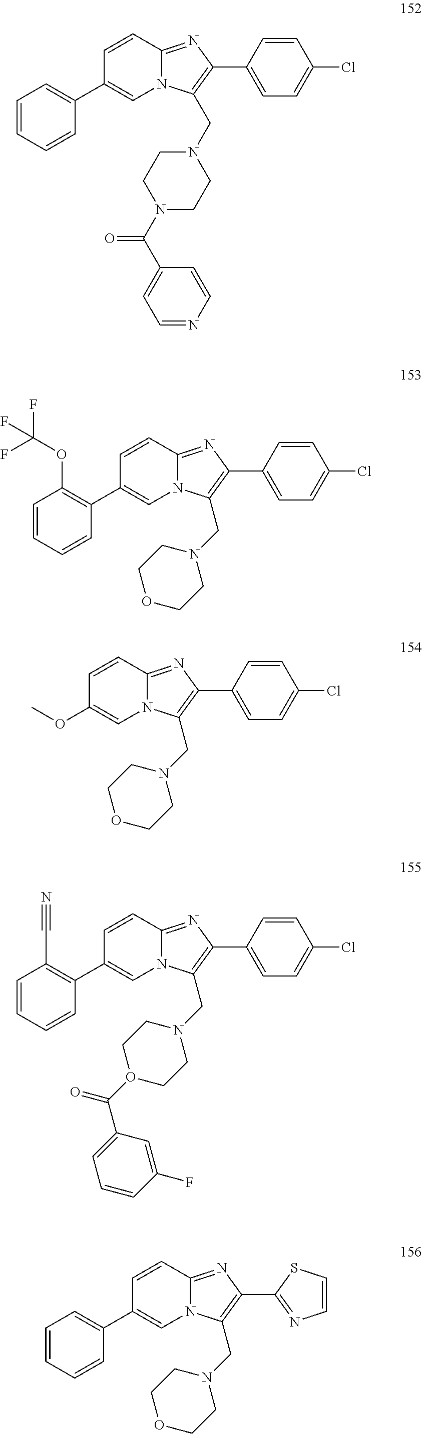

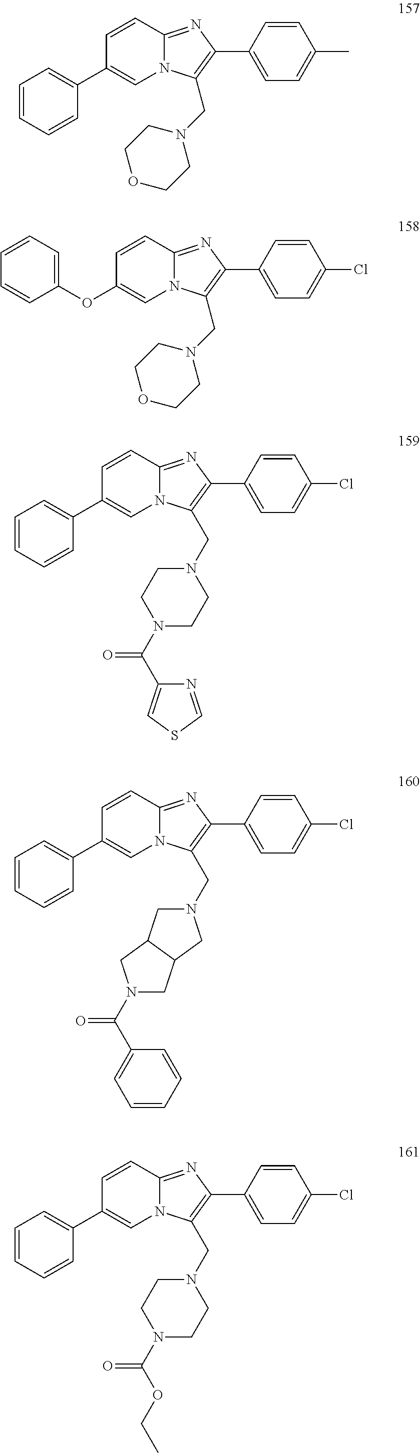 [SMILES: Cc1ccc(-c2nc3ccc(-c4ccccc4)cn3c2CN2CCOCC2)cc1 ; Clc1ccc(-c2nc3ccc(Oc4ccccc4)cn3c2CN2CCOCC2)cc1 ; O=C(c1cscn1)N1CCN(Cc2c(-c3ccc(Cl)cc3)nc3ccc(-c4ccccc4)cn23)CC1 ; O=C(c1ccccc1)N1CC2CN(Cc3c(-c4ccc(Cl)cc4)nc4ccc(-c5ccccc5)cn34)CC2C1 ; CCOC(=O)N1CCN(Cc2c(-c3ccc(Cl)cc3)nc3ccc(-c4ccccc4)cn23)CC1]

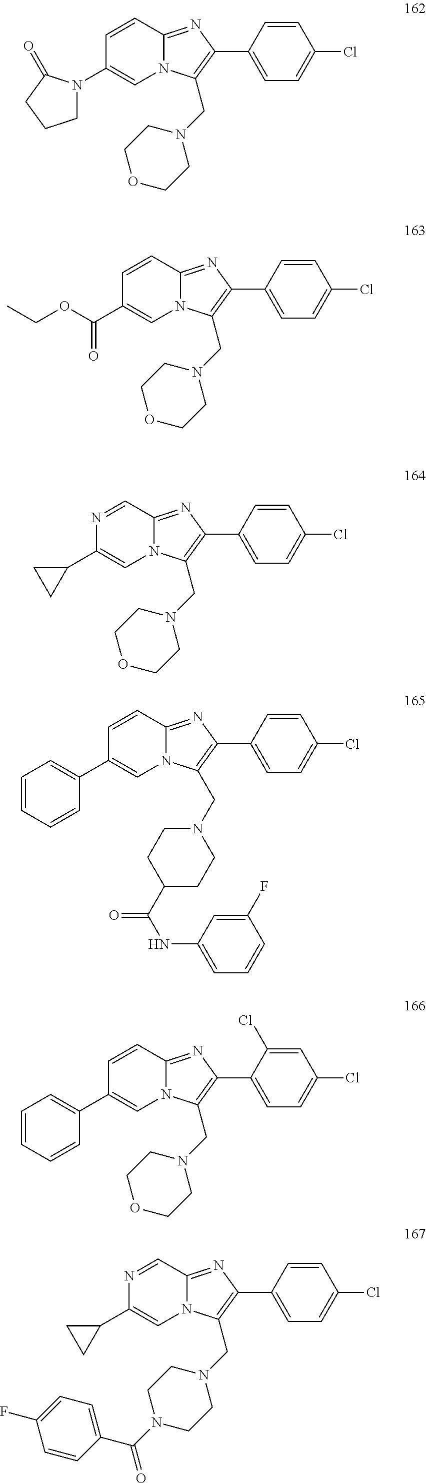

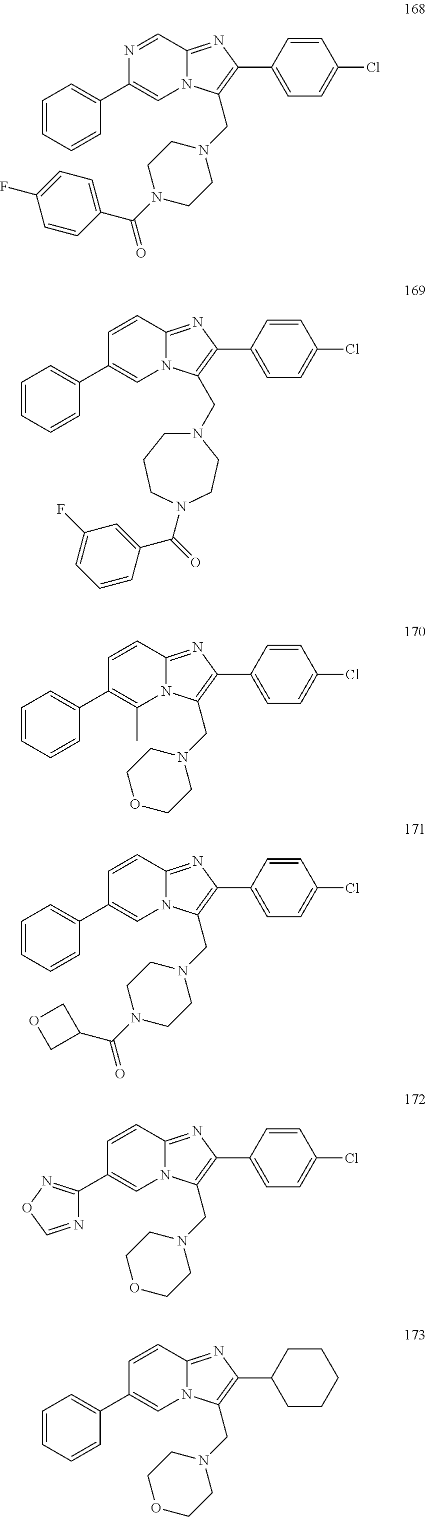 [SMILES: O=C(c1ccc(F)cc1)N1CCN(Cc2c(-c3ccc(Cl)cc3)nc3cnc(-c4ccccc4)cn23)CC1 ; O=C(c1cccc(F)c1)N1CCCN(Cc2c(-c3ccc(Cl)cc3)nc3ccc(-c4ccccc4)cn23)CC1 ; Cc1c(-c2ccccc2)ccc2nc(-c3ccc(Cl)cc3)c(CN3CCOCC3)n12 ; O=C(C1COC1)N1CCN(Cc2c(-c3ccc(Cl)cc3)nc3ccc(-c4ccccc4)cn23)CC1 ; Clc1ccc(-c2nc3ccc(-c4ncon4)cn3c2CN2CCOCC2)cc1 ; c1ccc(-c2ccc3nc(C4CCCCC4)c(CN4CCOCC4)n3c2)cc1]

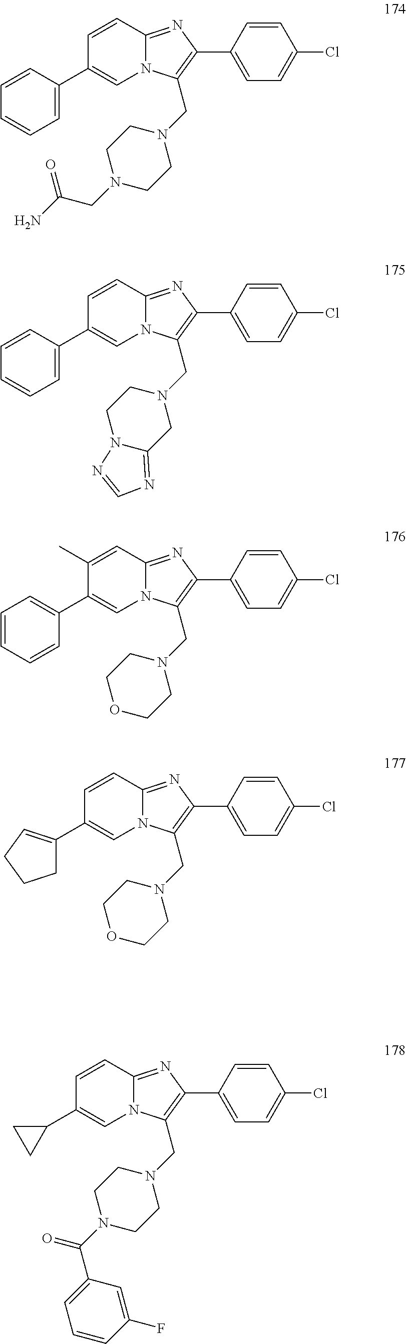 [SMILES: NC(=O)CN1CCN(Cc2c(-c3ccc(Cl)cc3)nc3ccc(-c4ccccc4)cn23)CC1 ; Clc1ccc(-c2nc3ccc(-c4ccccc4)cn3c2CN2CCn3ncnc3C2)cc1 ; Cc1cc2nc(-c3ccc(Cl)cc3)c(CN3CCOCC3)n2cc1-c1ccccc1 ; Clc1ccc(-c2nc3ccc(C4=CCCC4)cn3c2CN2CCOCC2)cc1 ; O=C(c1cccc(F)c1)N1CCN(Cc2c(-c3ccc(Cl)cc3)nc3ccc(C4CC4)cn23)CC1]

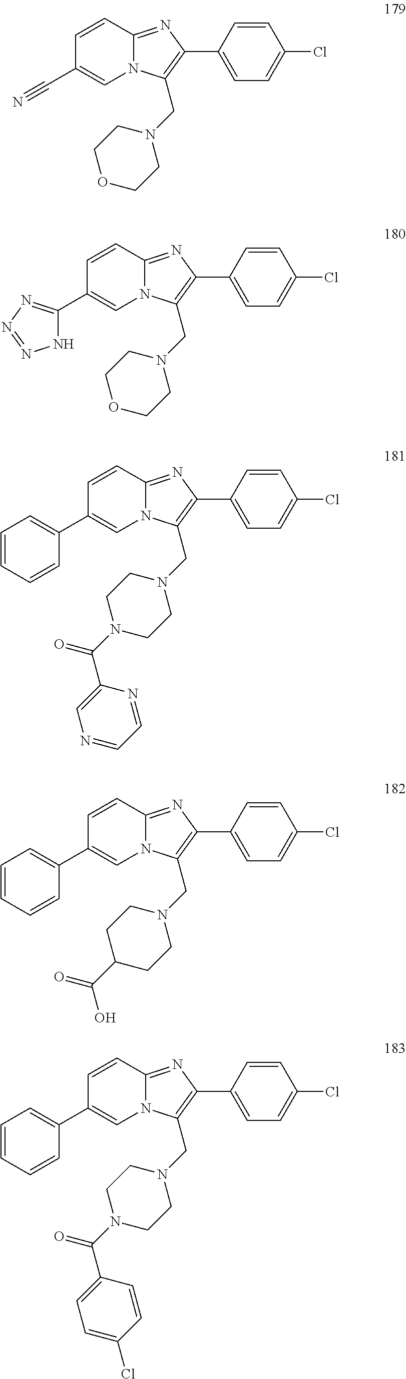

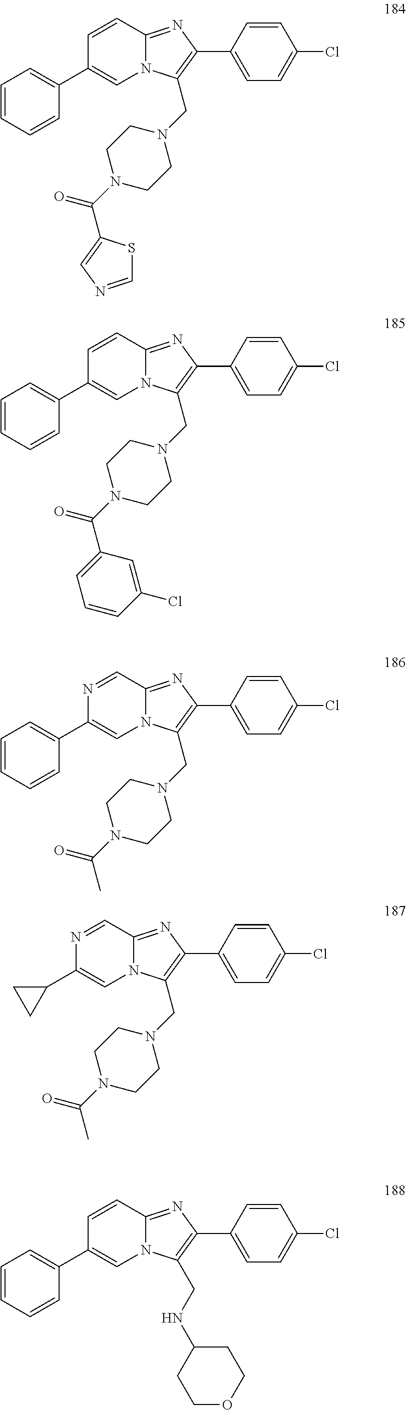

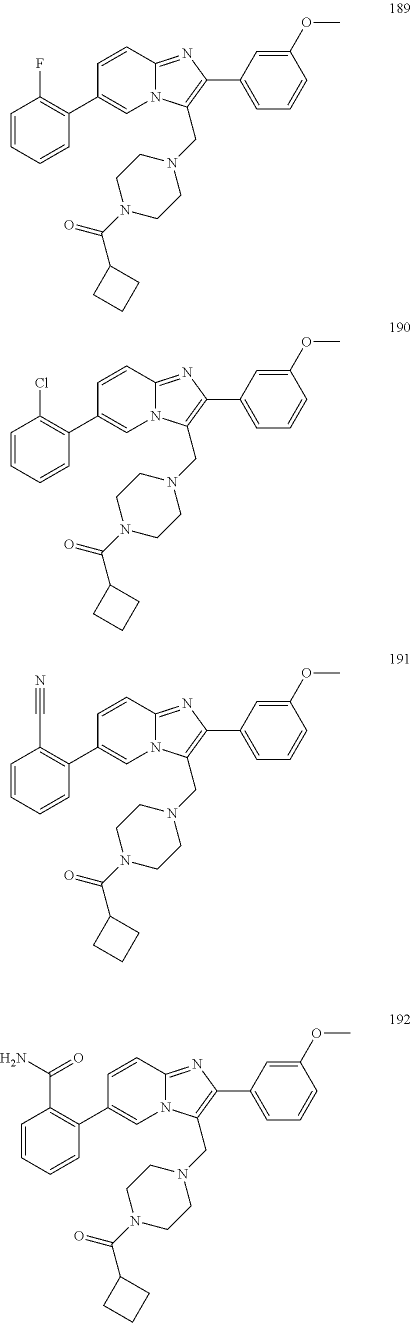 [SMILES: COc1cccc(-c2nc3ccc(-c4ccccc4F)cn3c2CN2CCN(C(=O)C3CCC3)CC2)c1 ; COc1cccc(-c2nc3ccc(-c4ccccc4Cl)cn3c2CN2CCN(C(=O)C3CCC3)CC2)c1 ; COc1cccc(-c2nc3ccc(-c4ccccc4C#N)cn3c2CN2CCN(C(=O)C3CCC3)CC2)c1 ; COc1cccc(-c2nc3ccc(-c4ccccc4C(N)=O)cn3c2CN2CCN(C(=O)C3CCC3)CC2)c1]

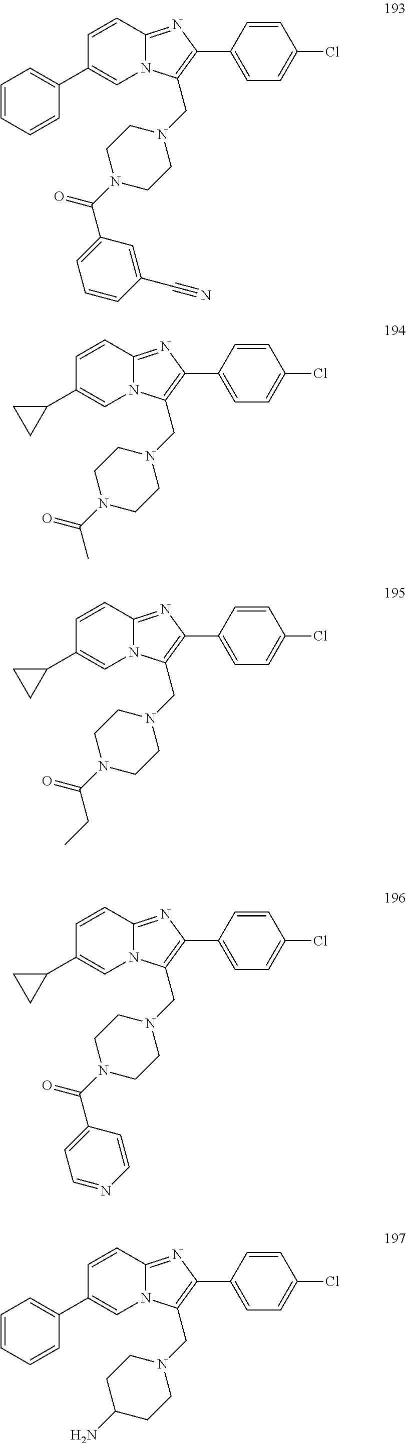

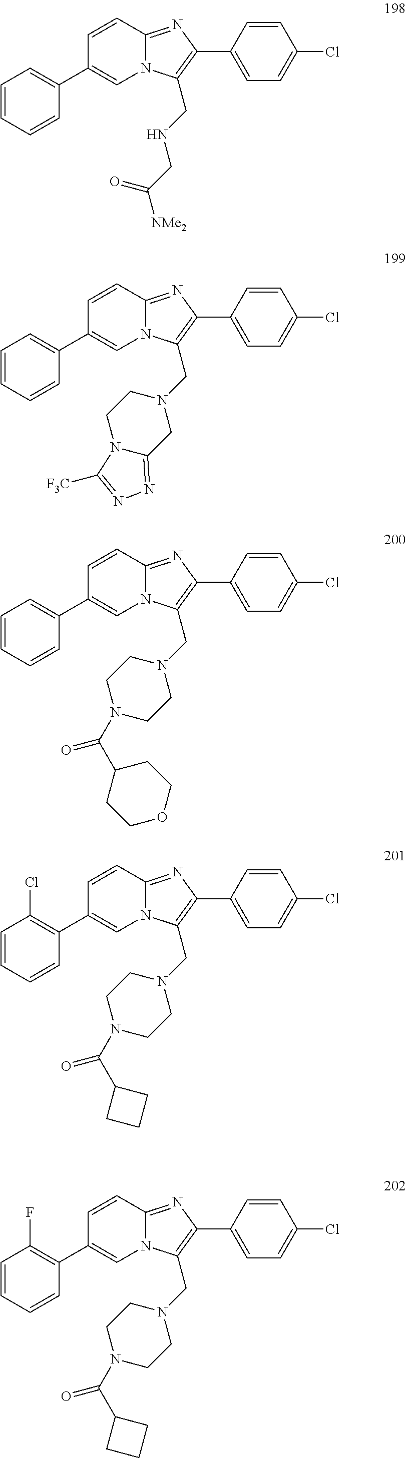

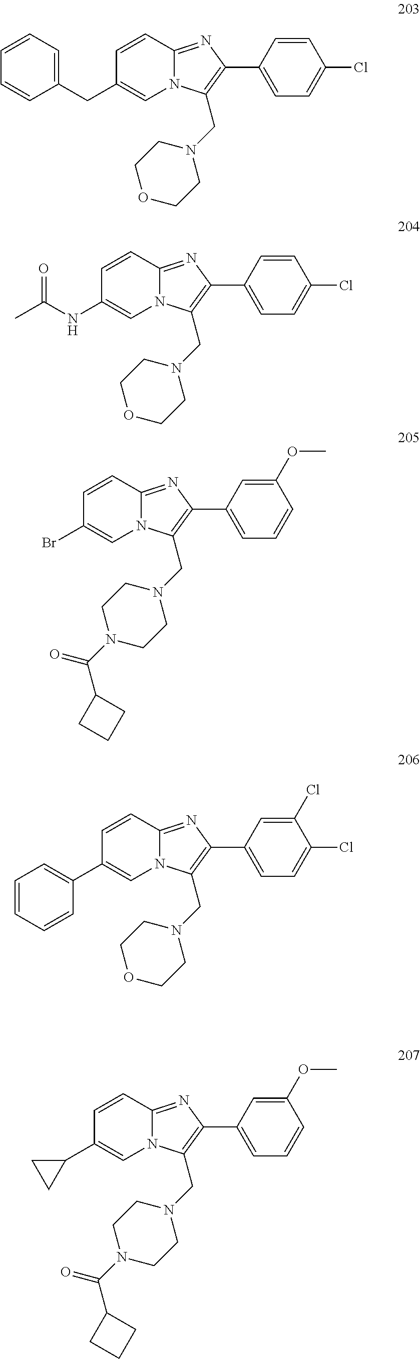 [SMILES: Clc1ccc(-c2nc3ccc(Cc4ccccc4)cn3c2CN2CCOCC2)cc1 ; CC(=O)Nc1ccc2nc(-c3ccc(Cl)cc3)c(CN3CCOCC3)n2c1 ; COc1cccc(-c2nc3ccc(Br)cn3c2CN2CCN(C(=O)C3CCC3)CC2)c1 ; Clc1ccc(-c2nc3ccc(-c4ccccc4)cn3c2CN2CCOCC2)cc1Cl ; COc1cccc(-c2nc3ccc(C4CC4)cn3c2CN2CCN(C(=O)C3CCC3)CC2)c1]

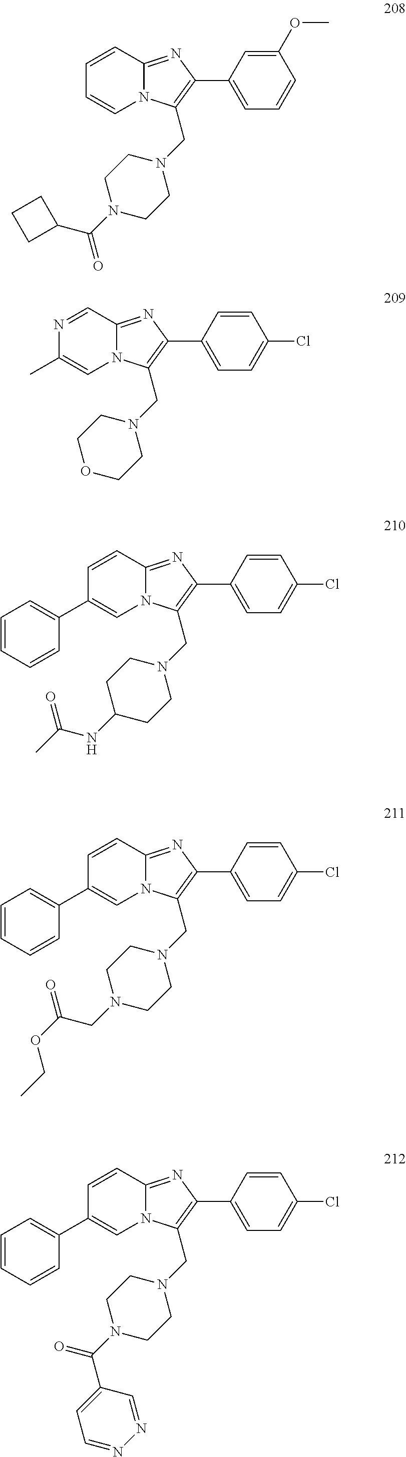

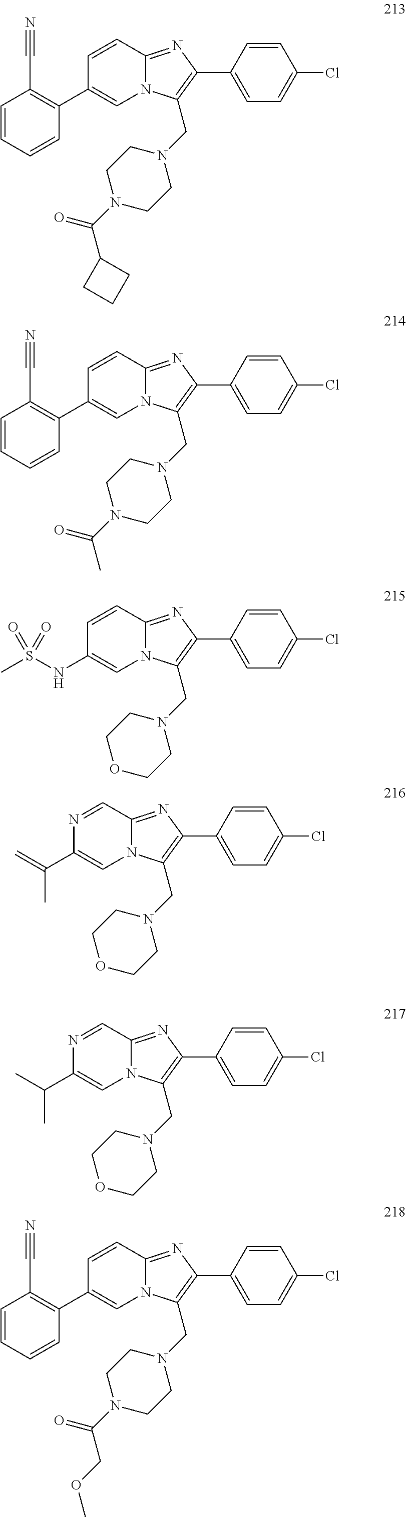

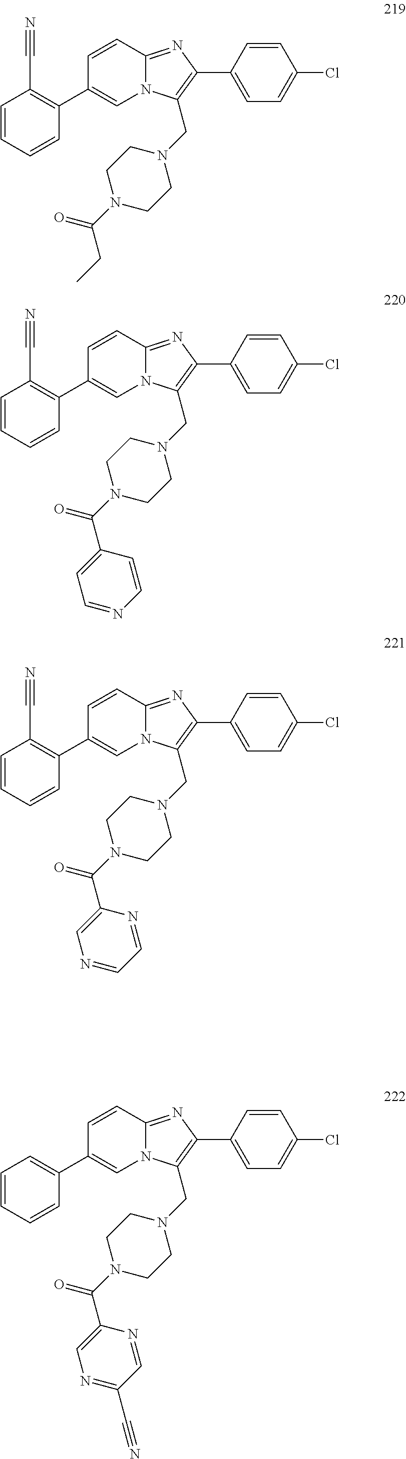 [SMILES: CCC(=O)N1CCN(Cc2c(-c3ccc(Cl)cc3)nc3ccc(-c4ccccc4C#N)cn23)CC1 ; N#Cc1ccccc1-c1ccc2nc(-c3ccc(Cl)cc3)c(CN3CCN(C(=O)c4ccncc4)CC3)n2c1 ; N#Cc1ccccc1-c1ccc2nc(-c3ccc(Cl)cc3)c(CN3CCN(C(=O)c4cnccn4)CC3)n2c1 ; N#Cc1cnc(C(=O)N2CCN(Cc3c(-c4ccc(Cl)cc4)nc4ccc(-c5ccccc5)cn34)CC2)cn1]

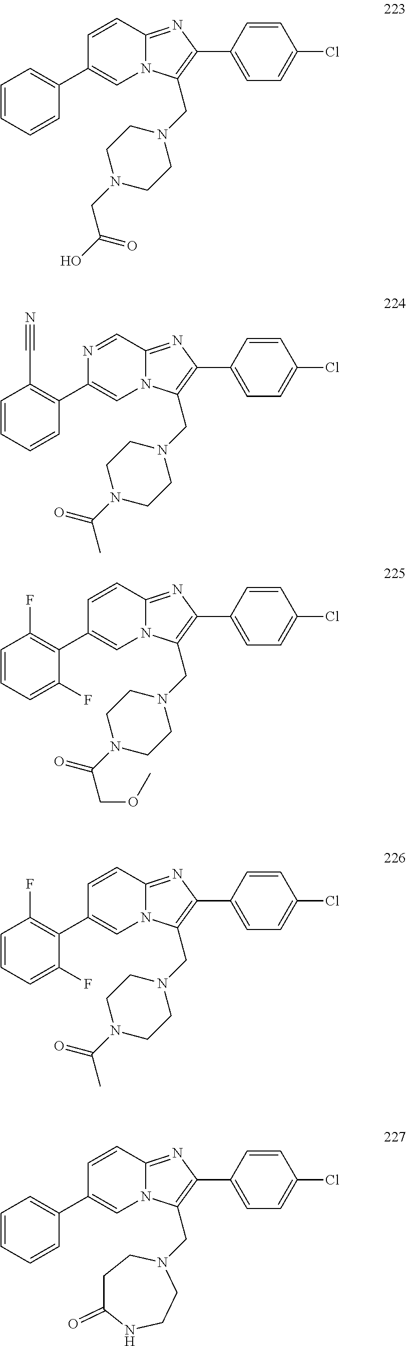

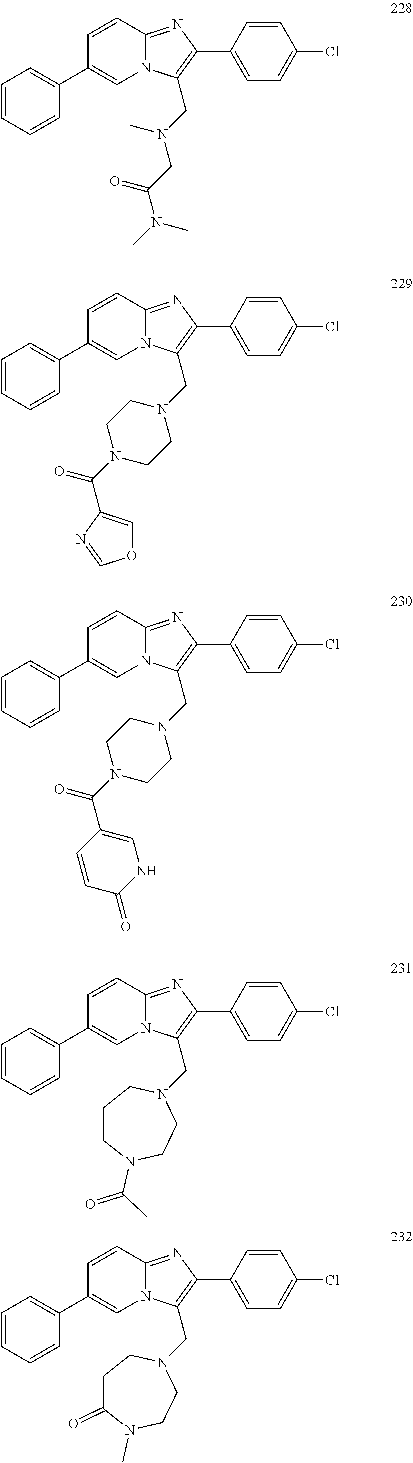

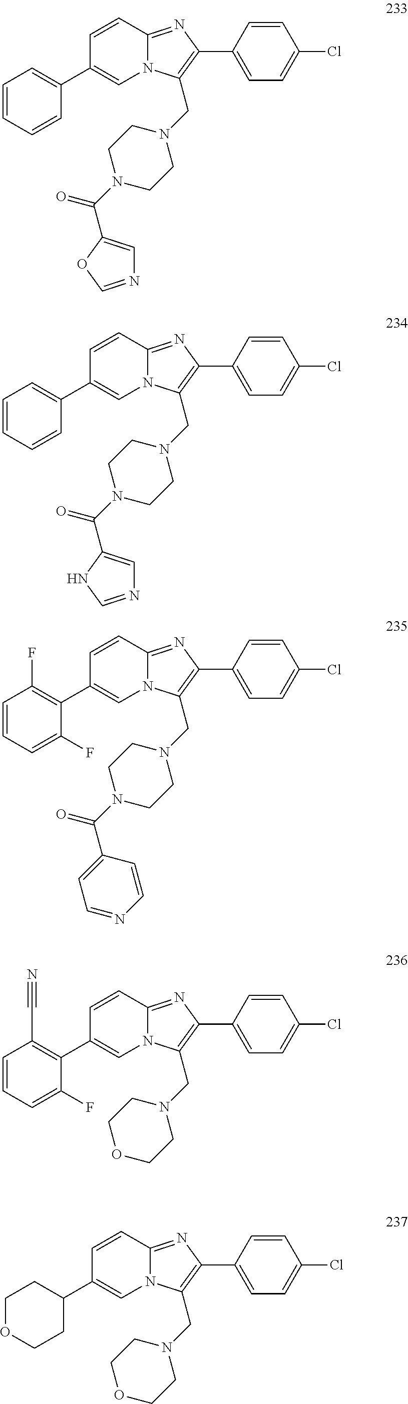

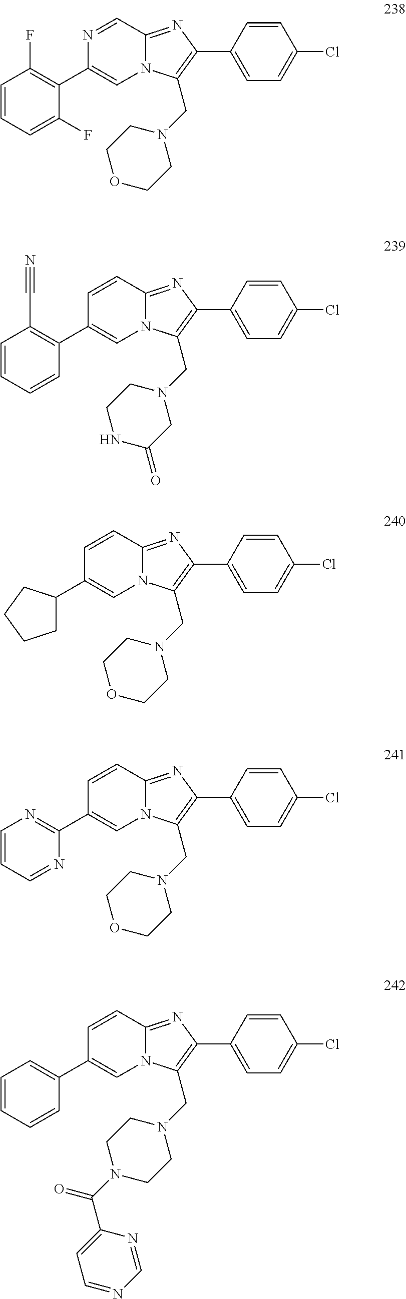 [SMILES: Fc1cccc(F)c1-c1cn2c(CN3CCOCC3)c(-c3ccc(Cl)cc3)nc2cn1 ; N#Cc1ccccc1-c1ccc2nc(-c3ccc(Cl)cc3)c(CN3CCNC(=O)C3)n2c1 ; Clc1ccc(-c2nc3ccc(C4CCCC4)cn3c2CN2CCOCC2)cc1 ; Clc1ccc(-c2nc3ccc(-c4ncccn4)cn3c2CN2CCOCC2)cc1 ; O=C(c1ccncn1)N1CCN(Cc2c(-c3ccc(Cl)cc3)nc3ccc(-c4ccccc4)cn23)CC1]

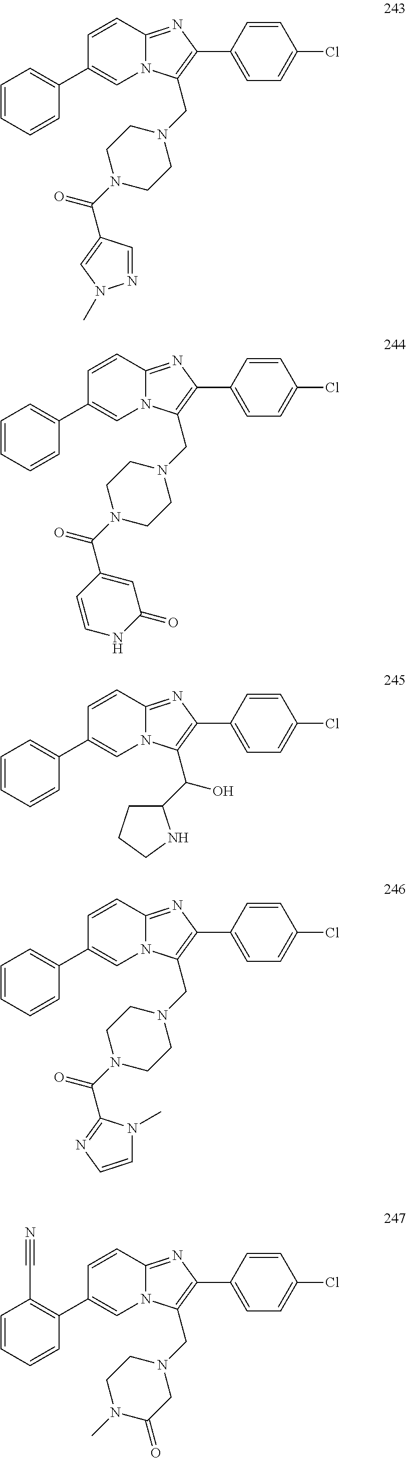

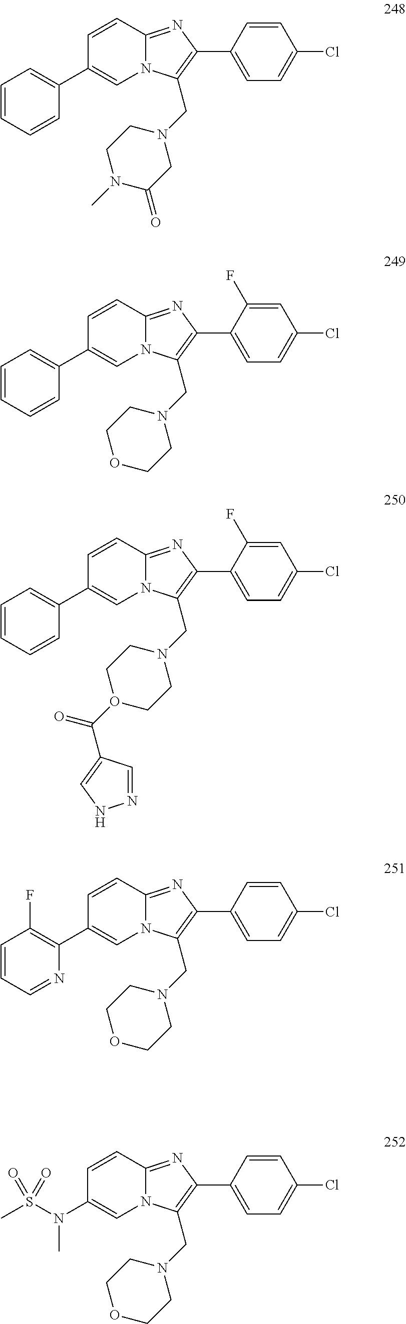

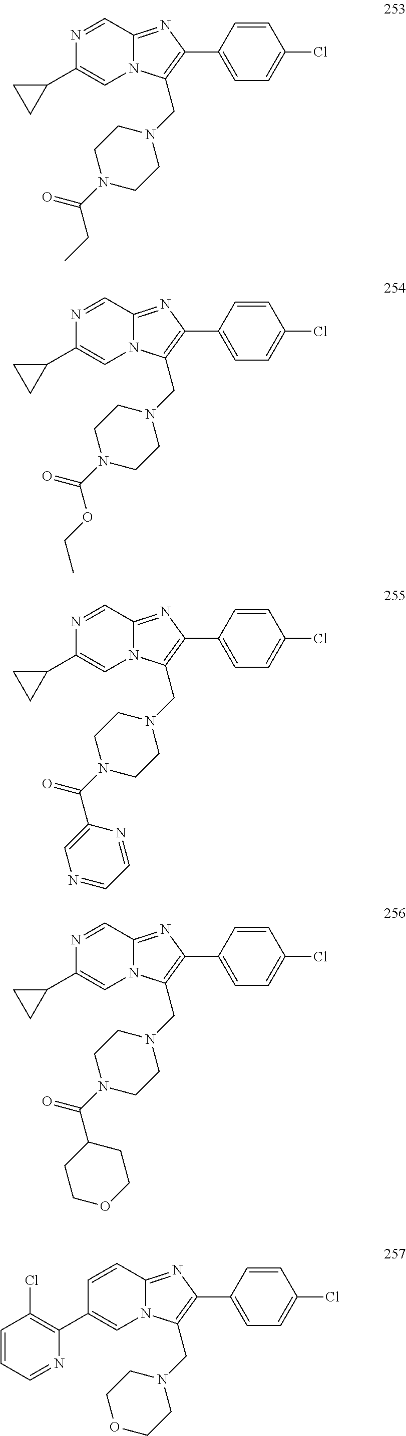

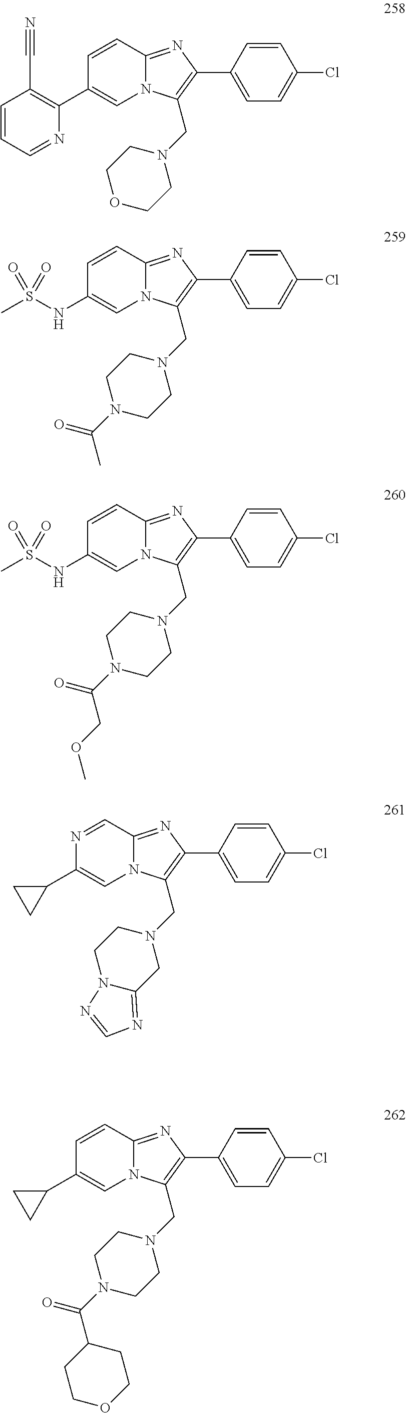

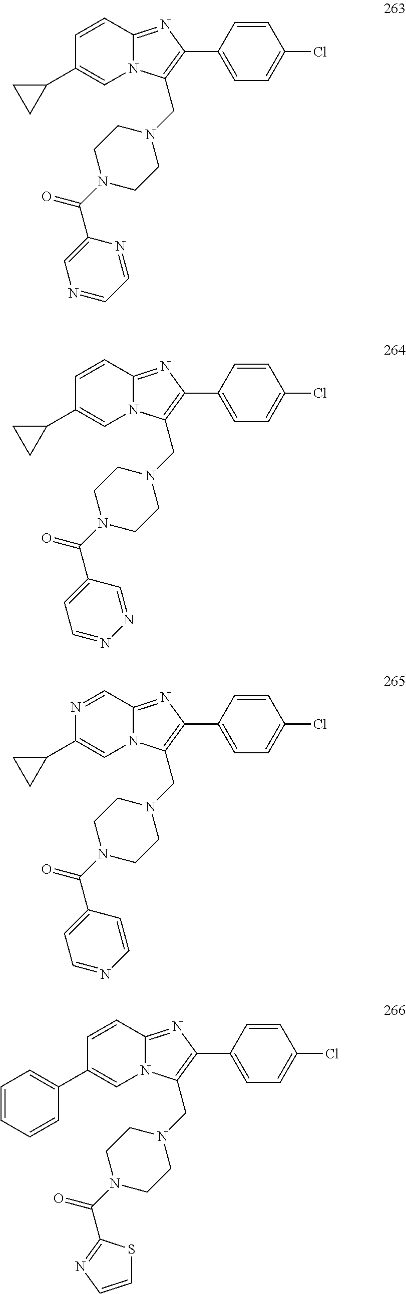 [SMILES: O=C(c1cnccn1)N1CCN(Cc2c(-c3ccc(Cl)cc3)nc3ccc(C4CC4)cn23)CC1 ; O=C(c1ccnnc1)N1CCN(Cc2c(-c3ccc(Cl)cc3)nc3ccc(C4CC4)cn23)CC1 ; O=C(c1ccncc1)N1CCN(Cc2c(-c3ccc(Cl)cc3)nc3cnc(C4CC4)cn23)CC1 ; O=C(c1nccs1)N1CCN(Cc2c(-c3ccc(Cl)cc3)nc3ccc(-c4ccccc4)cn23)CC1]

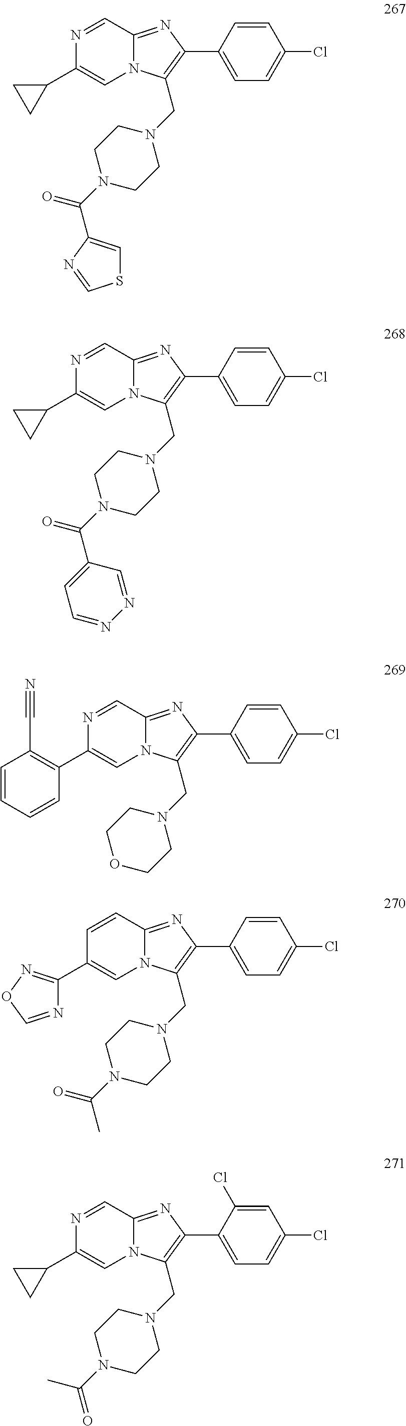 [SMILES: O=C(c1cscn1)N1CCN(Cc2c(-c3ccc(Cl)cc3)nc3cnc(C4CC4)cn23)CC1 ; O=C(c1ccnnc1)N1CCN(Cc2c(-c3ccc(Cl)cc3)nc3cnc(C4CC4)cn23)CC1 ; N#Cc1ccccc1-c1cn2c(CN3CCOCC3)c(-c3ccc(Cl)cc3)nc2cn1 ; CC(=O)N1CCN(Cc2c(-c3ccc(Cl)cc3)nc3ccc(-c4ncon4)cn23)CC1 ; CC(=O)N1CCN(Cc2c(-c3ccc(Cl)cc3Cl)nc3cnc(C4CC4)cn23)CC1]

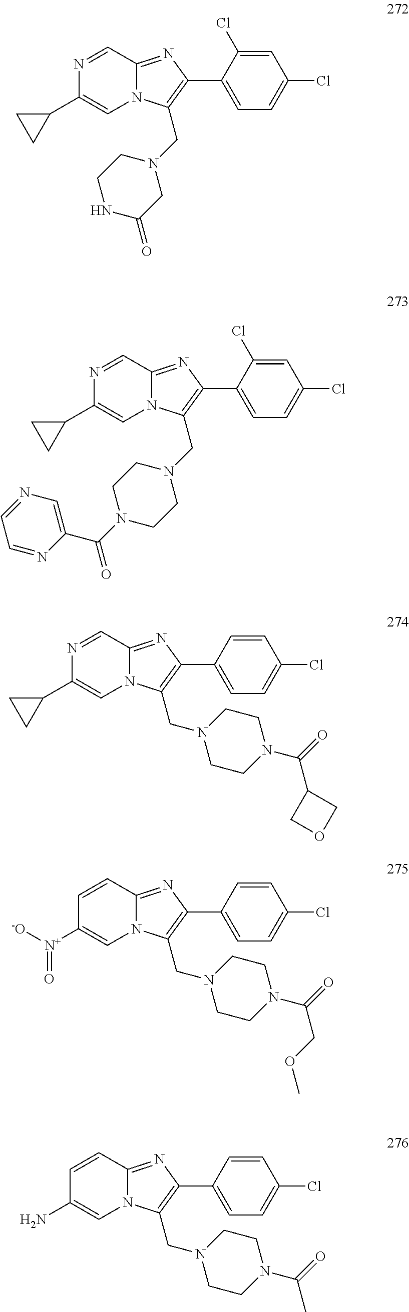

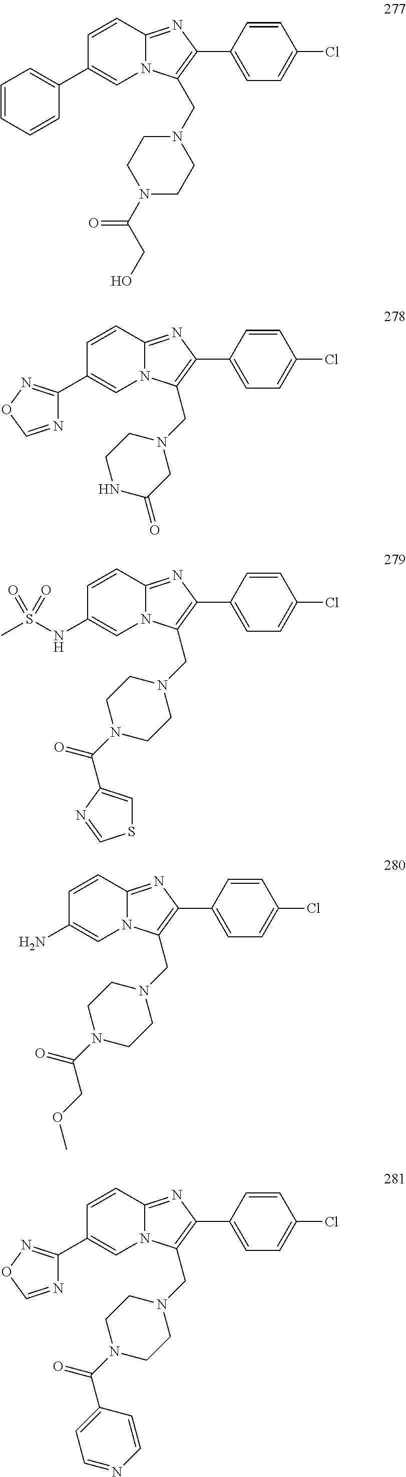

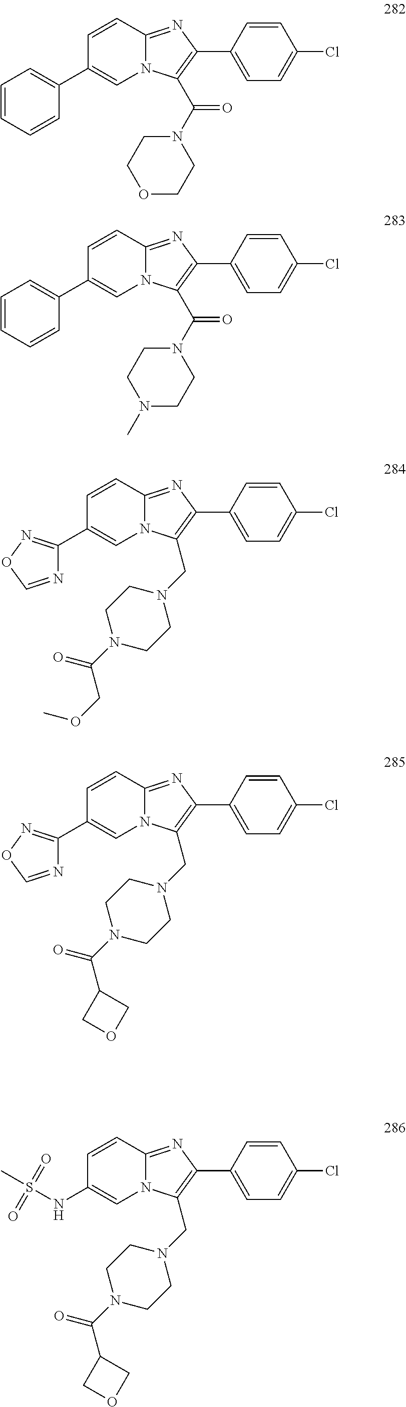

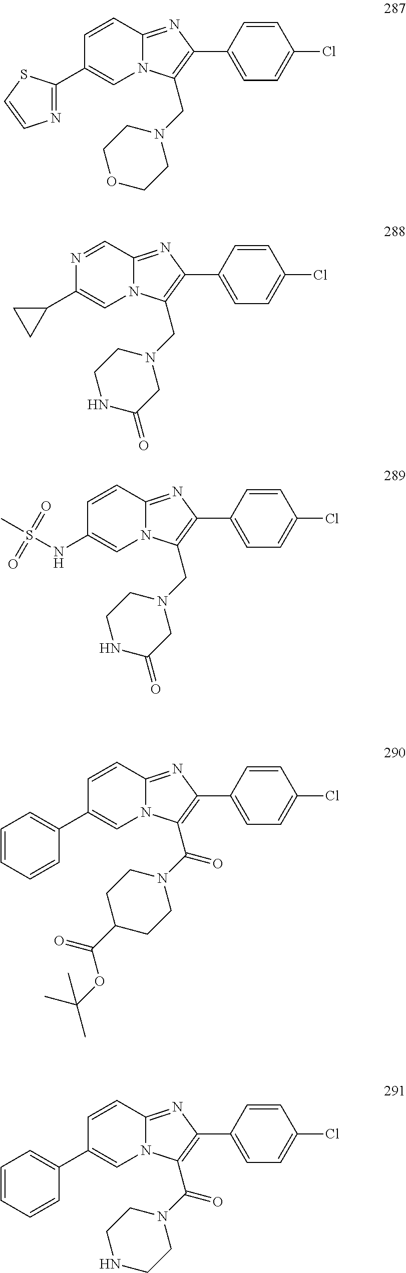 [SMILES: Clc1ccc(-c2nc3ccc(-c4nccs4)cn3c2CN2CCOCC2)cc1 ; O=C1CN(Cc2c(-c3ccc(Cl)cc3)nc3cnc(C4CC4)cn23)CCN1 ; CS(=O)(=O)Nc1ccc2nc(-c3ccc(Cl)cc3)c(CN3CCNC(=O)C3)n2c1 ; CC(C)(C)OC(=O)C1CCN(C(=O)c2c(-c3ccc(Cl)cc3)nc3ccc(-c4ccccc4)cn23)CC1 ; O=C(c1c(-c2ccc(Cl)cc2)nc2ccc(-c3ccccc3)cn12)N1CCNCC1]

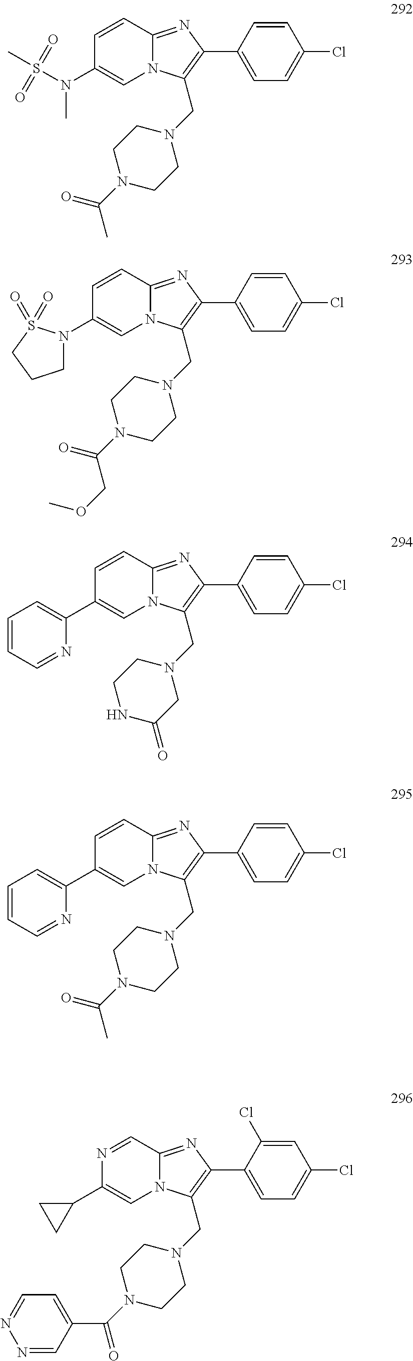

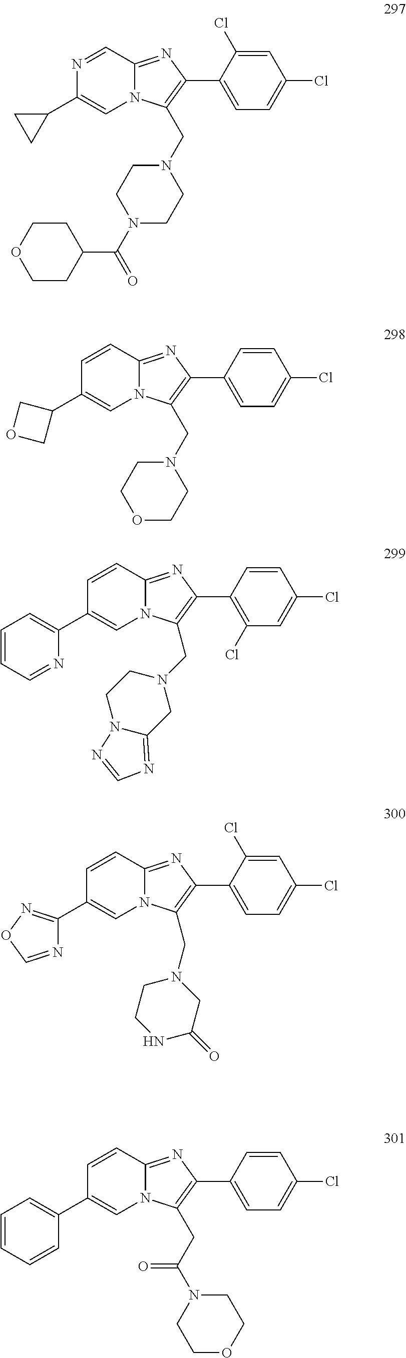 [SMILES: O=C(C1CCOCC1)N1CCN(Cc2c(-c3ccc(Cl)cc3Cl)nc3cnc(C4CC4)cn23)CC1 ; Clc1ccc(-c2nc3ccc(C4COC4)cn3c2CN2CCOCC2)cc1 ; Clc1ccc(-c2nc3ccc(-c4ccccn4)cn3c2CN2CCn3ncnc3C2)c(Cl)c1 ; O=C1CN(Cc2c(-c3ccc(Cl)cc3Cl)nc3ccc(-c4ncon4)cn23)CCN1 ; O=C(Cc1c(-c2ccc(Cl)cc2)nc2ccc(-c3ccccc3)cn12)N1CCOCC1]

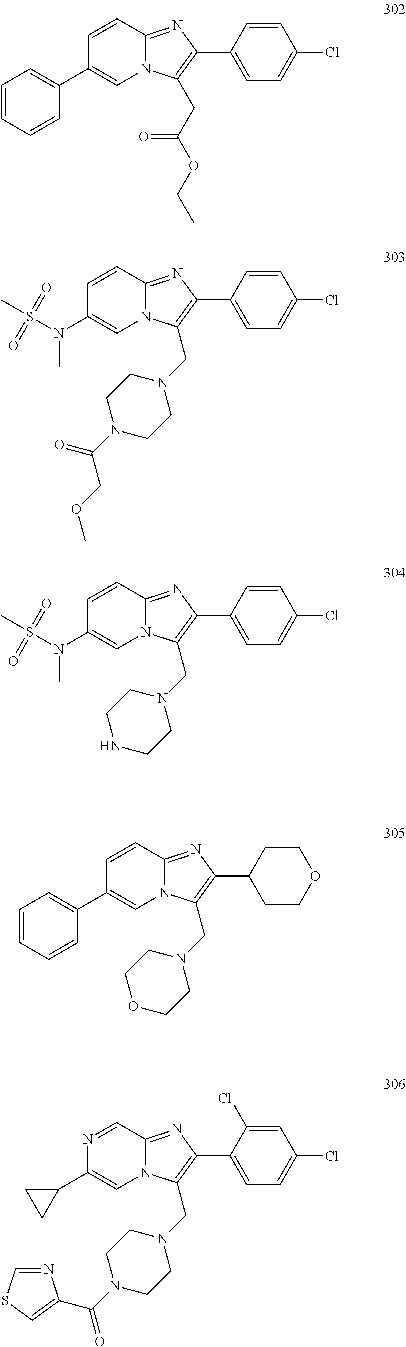 [SMILES: CCOC(=O)Cc1c(-c2ccc(Cl)cc2)nc2ccc(-c3ccccc3)cn12 ; COCC(=O)N1CCN(Cc2c(-c3ccc(Cl)cc3)nc3ccc(N(C)S(C)(=O)=O)cn23)CC1 ; CN(c1ccc2nc(-c3ccc(Cl)cc3)c(CN3CCNCC3)n2c1)S(C)(=O)=O ; c1ccc(-c2ccc3nc(C4CCOCC4)c(CN4CCOCC4)n3c2)cc1 ; O=C(c1cscn1)N1CCN(Cc2c(-c3ccc(Cl)cc3Cl)nc3cnc(C4CC4)cn23)CC1]

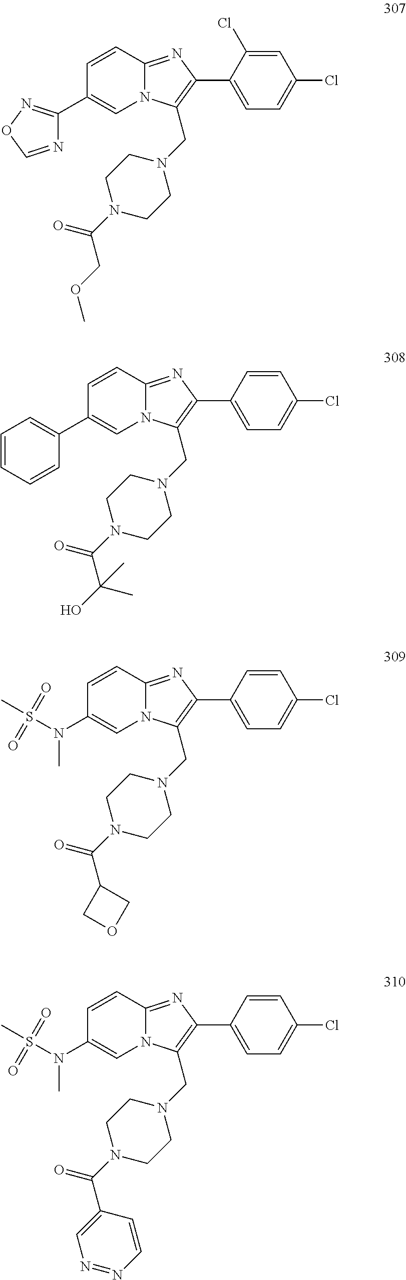 [SMILES: COCC(=O)N1CCN(Cc2c(-c3ccc(Cl)cc3Cl)nc3ccc(-c4ncon4)cn23)CC1 ; CC(C)(O)C(=O)N1CCN(Cc2c(-c3ccc(Cl)cc3)nc3ccc(-c4ccccc4)cn23)CC1 ; CN(c1ccc2nc(-c3ccc(Cl)cc3)c(CN3CCN(C(=O)C4COC4)CC3)n2c1)S(C)(=O)=O ; CN(c1ccc2nc(-c3ccc(Cl)cc3)c(CN3CCN(C(=O)c4ccnnc4)CC3)n2c1)S(C)(=O)=O]

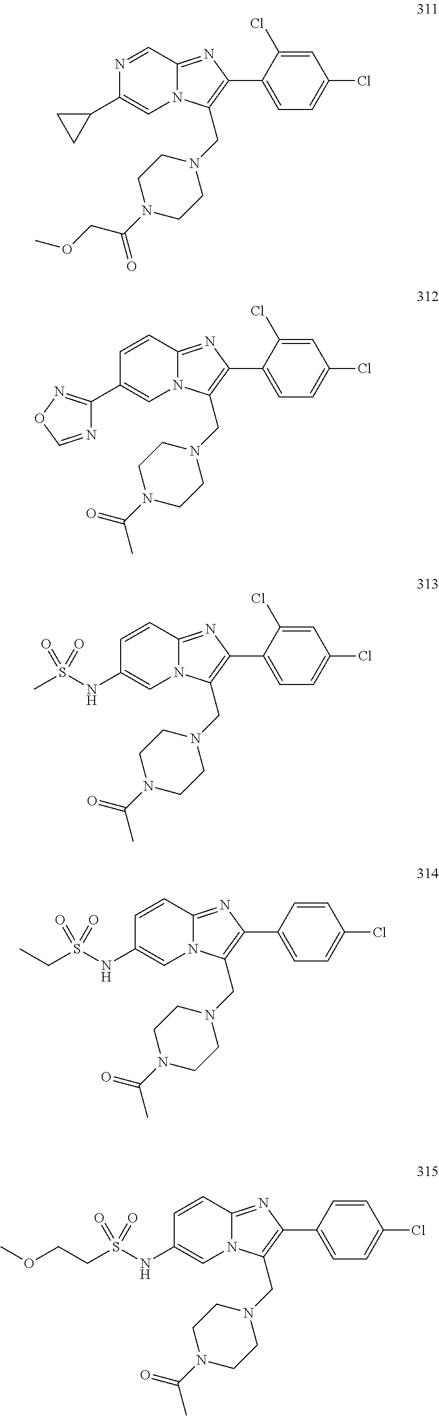 [SMILES: COCC(=O)N1CCN(Cc2c(-c3ccc(Cl)cc3Cl)nc3cnc(C4CC4)cn23)CC1 ; CC(=O)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(NS(C)(=O)=O)cn23)CC1 ; CCS(=O)(=O)Nc1ccc2nc(-c3ccc(Cl)cc3)c(CN3CCN(C(C)=O)CC3)n2c1 ; COCCS(=O)(=O)Nc1ccc2nc(-c3ccc(Cl)cc3)c(CN3CCN(C(C)=O)CC3)n2c1]

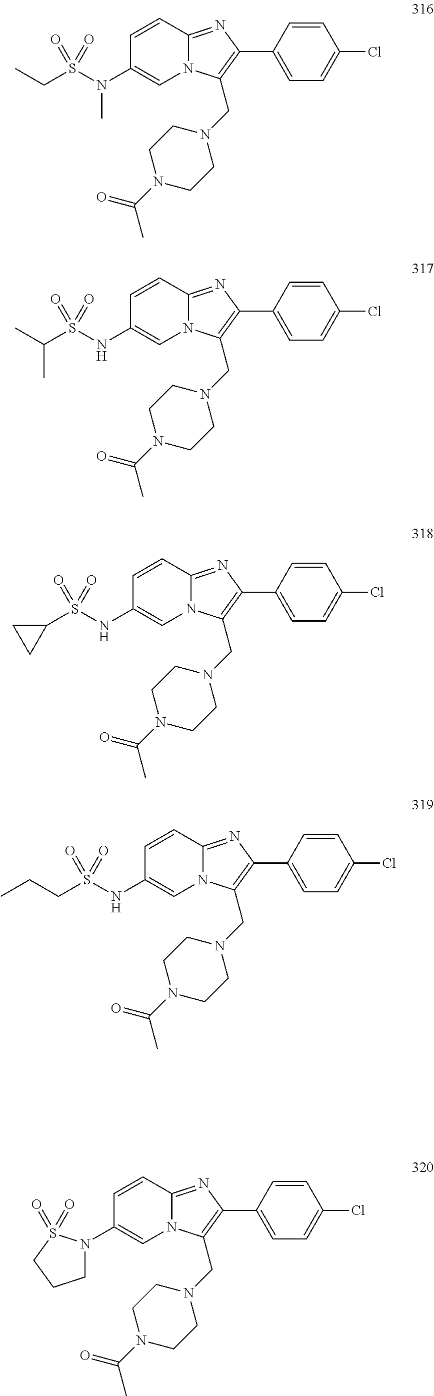

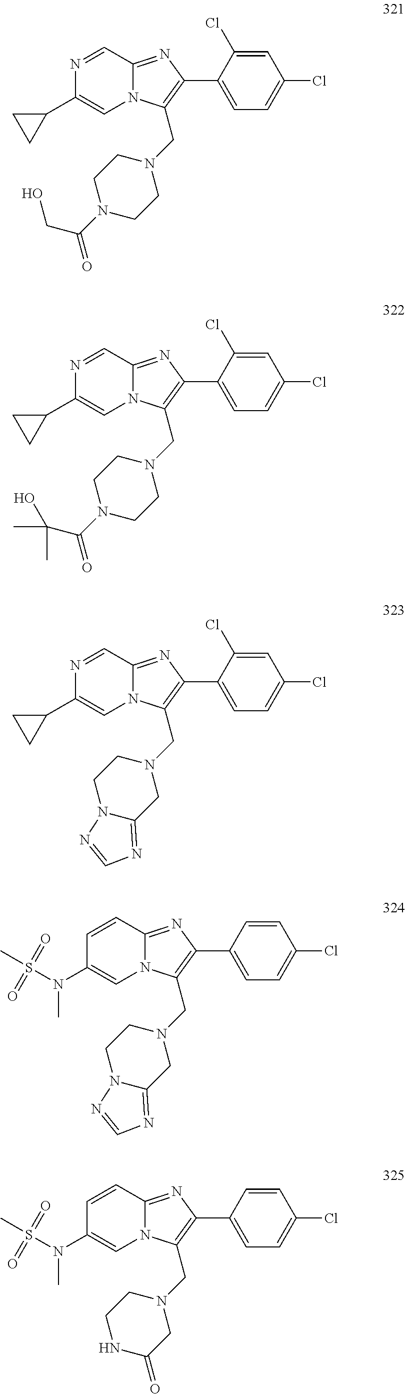 [SMILES: O=C(CO)N1CCN(Cc2c(-c3ccc(Cl)cc3Cl)nc3cnc(C4CC4)cn23)CC1 ; CC(C)(O)C(=O)N1CCN(Cc2c(-c3ccc(Cl)cc3Cl)nc3cnc(C4CC4)cn23)CC1 ; Clc1ccc(-c2nc3cnc(C4CC4)cn3c2CN2CCn3ncnc3C2)c(Cl)c1 ; CN(c1ccc2nc(-c3ccc(Cl)cc3)c(CN3CCn4ncnc4C3)n2c1)S(C)(=O)=O ; CN(c1ccc2nc(-c3ccc(Cl)cc3)c(CN3CCNC(=O)C3)n2c1)S(C)(=O)=O]

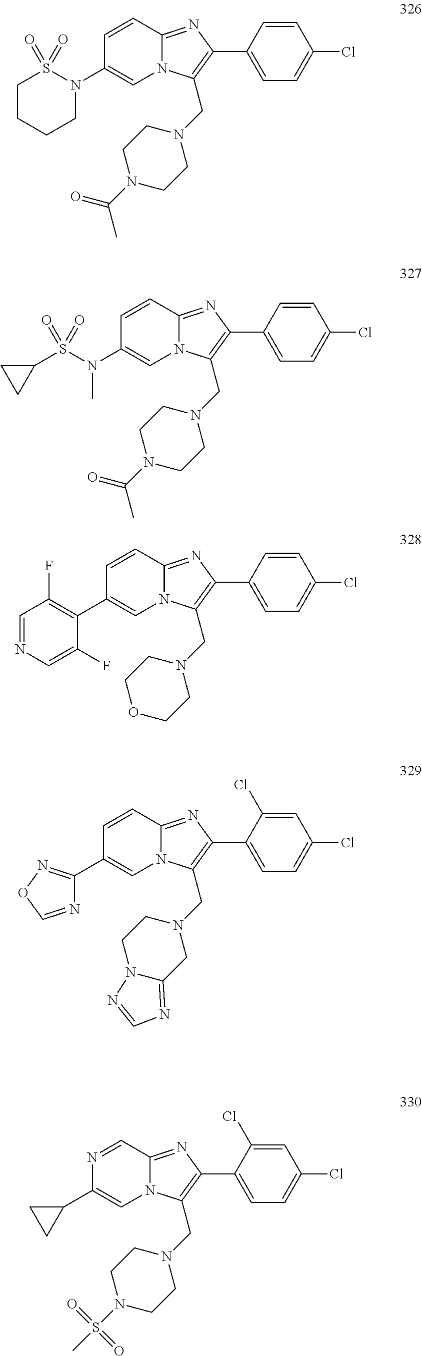

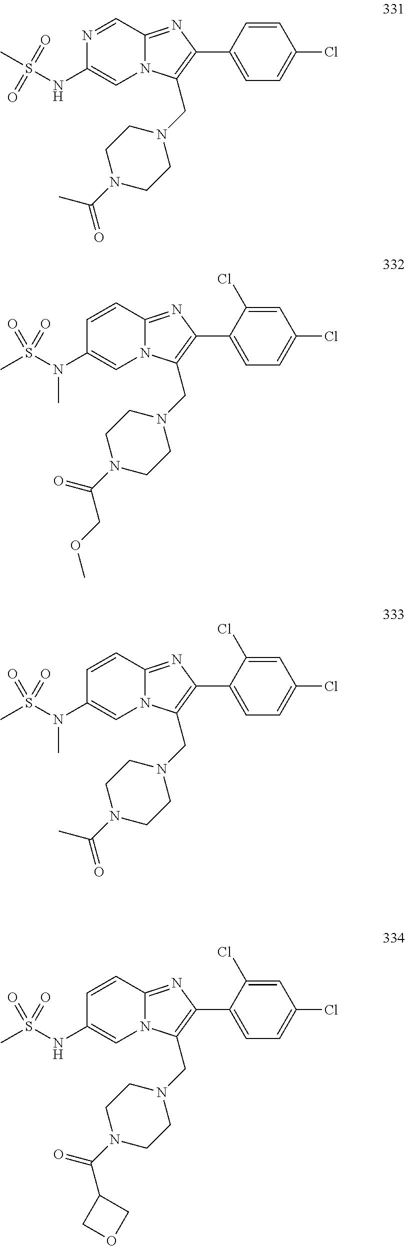 [SMILES: CC(=O)N1CCN(Cc2c(-c3ccc(Cl)cc3)nc3cnc(NS(C)(=O)=O)cn23)CC1 ; COCC(=O)N1CCN(Cc2c(-c3ccc(Cl)cc3Cl)nc3ccc(N(C)S(C)(=O)=O)cn23)CC1 ; CC(=O)N1CCN(Cc2c(-c3ccc(Cl)cc3Cl)nc3ccc(N(C)S(C)(=O)=O)cn23)CC1 ; CS(=O)(=O)Nc1ccc2nc(-c3ccc(Cl)cc3Cl)c(CN3CCN(C(=O)C4COC4)CC3)n2c1]

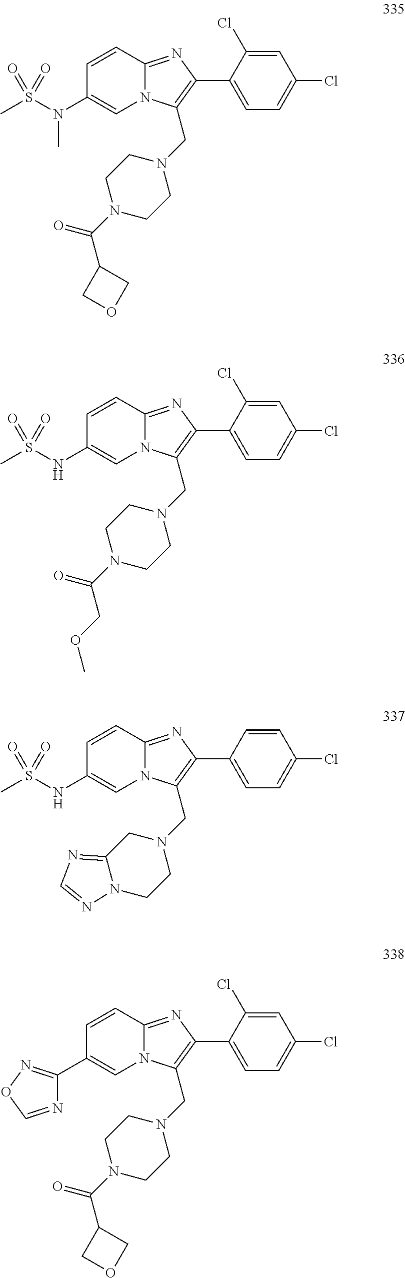 [SMILES: CN(c1ccc2nc(-c3ccc(Cl)cc3Cl)c(CN3CCN(C(=O)C4COC4)CC3)n2c1)S(C)(=O)=O ; COCC(=O)N1CCN(Cc2c(-c3ccc(Cl)cc3Cl)nc3ccc(NS(C)(=O)=O)cn23)CC1 ; CS(=O)(=O)Nc1ccc2nc(-c3ccc(Cl)cc3)c(CN3CCn4ncnc4C3)n2c1 ; O=C(C1COC1)N1CCN(Cc2c(-c3ccc(Cl)cc3Cl)nc3ccc(-c4ncon4)cn23)CC1]

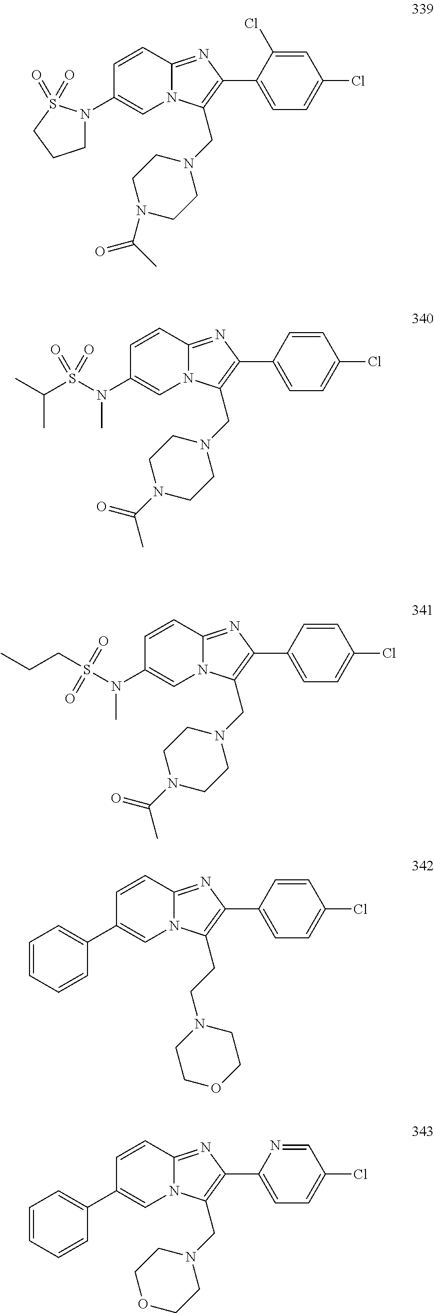

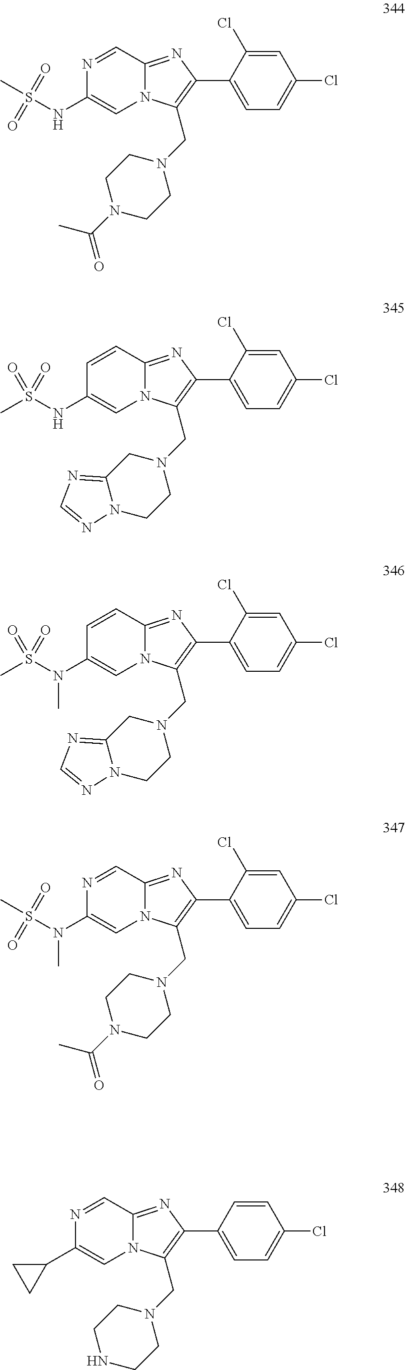 [SMILES: CC(=O)N1CCN(Cc2c(-c3ccc(Cl)cc3Cl)nc3cnc(NS(C)(=O)=O)cn23)CC1 ; CS(=O)(=O)Nc1ccc2nc(-c3ccc(Cl)cc3Cl)c(CN3CCn4ncnc4C3)n2c1 ; CN(c1ccc2nc(-c3ccc(Cl)cc3Cl)c(CN3CCn4ncnc4C3)n2c1)S(C)(=O)=O ; CC(=O)N1CCN(Cc2c(-c3ccc(Cl)cc3Cl)nc3cnc(N(C)S(C)(=O)=O)cn23)CC1 ; Clc1ccc(-c2nc3cnc(C4CC4)cn3c2CN2CCNCC2)cc1]

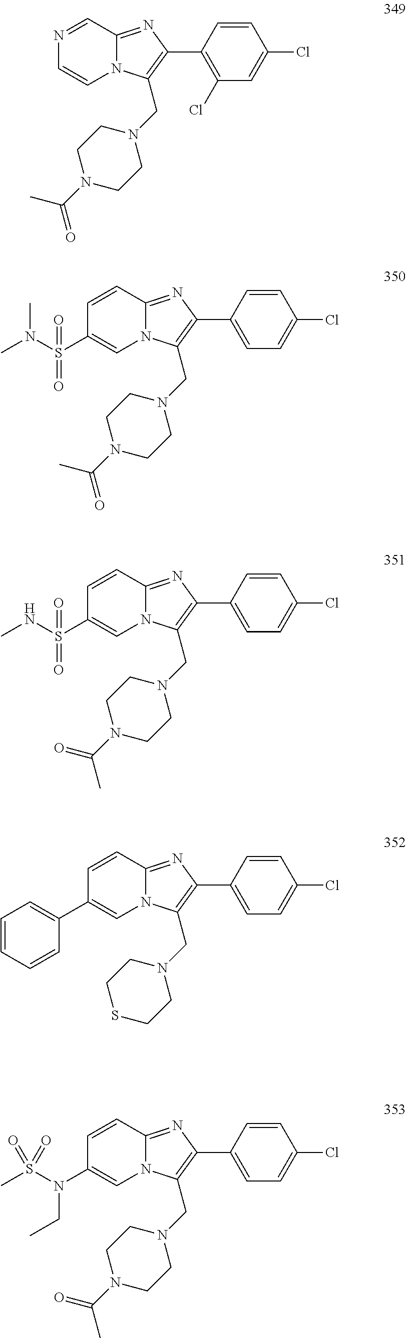 [SMILES: CC(=O)N1CCN(Cc2c(-c3ccc(Cl)cc3Cl)nc3cnccn23)CC1 ; CC(=O)N1CCN(Cc2c(-c3ccc(Cl)cc3)nc3ccc(S(=O)(=O)N(C)C)cn23)CC1 ; CNS(=O)(=O)c1ccc2nc(-c3ccc(Cl)cc3)c(CN3CCN(C(C)=O)CC3)n2c1 ; Clc1ccc(-c2nc3ccc(-c4ccccc4)cn3c2CN2CCSCC2)cc1 ; CCN(c1ccc2nc(-c3ccc(Cl)cc3)c(CN3CCN(C(C)=O)CC3)n2c1)S(C)(=O)=O]

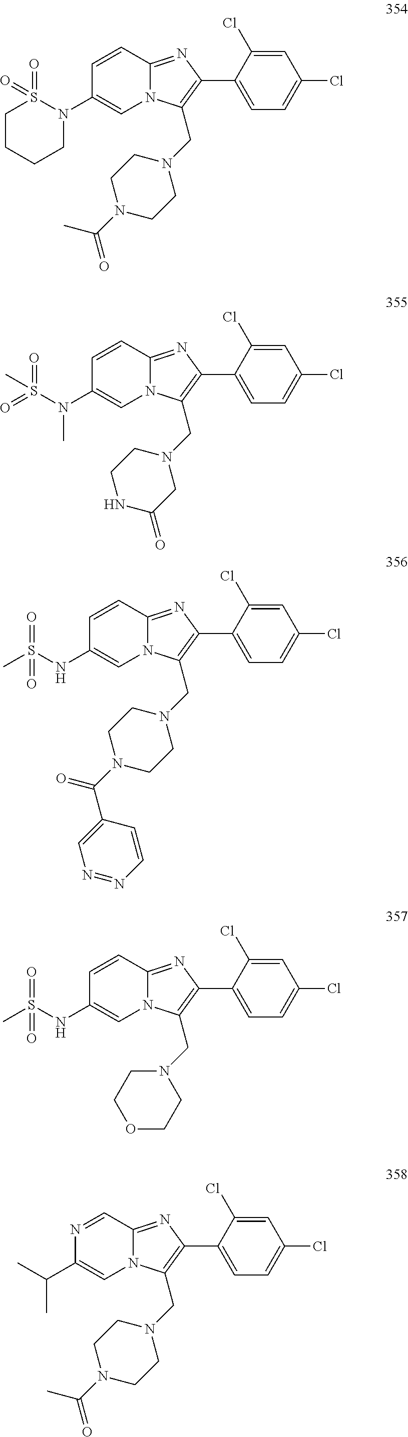 [SMILES: CC(=O)N1CCN(Cc2c(-c3ccc(Cl)cc3Cl)nc3ccc(N4CCCCS4(=O)=O)cn23)CC1 ; CN(c1ccc2nc(-c3ccc(Cl)cc3Cl)c(CN3CCNC(=O)C3)n2c1)S(C)(=O)=O ; CS(=O)(=O)Nc1ccc2nc(-c3ccc(Cl)cc3Cl)c(CN3CCN(C(=O)c4ccnnc4)CC3)n2c1 ; CS(=O)(=O)Nc1ccc2nc(-c3ccc(Cl)cc3Cl)c(CN3CCOCC3)n2c1 ; CC(=O)N1CCN(Cc2c(-c3ccc(Cl)cc3Cl)nc3cnc(C(C)C)cn23)CC1]

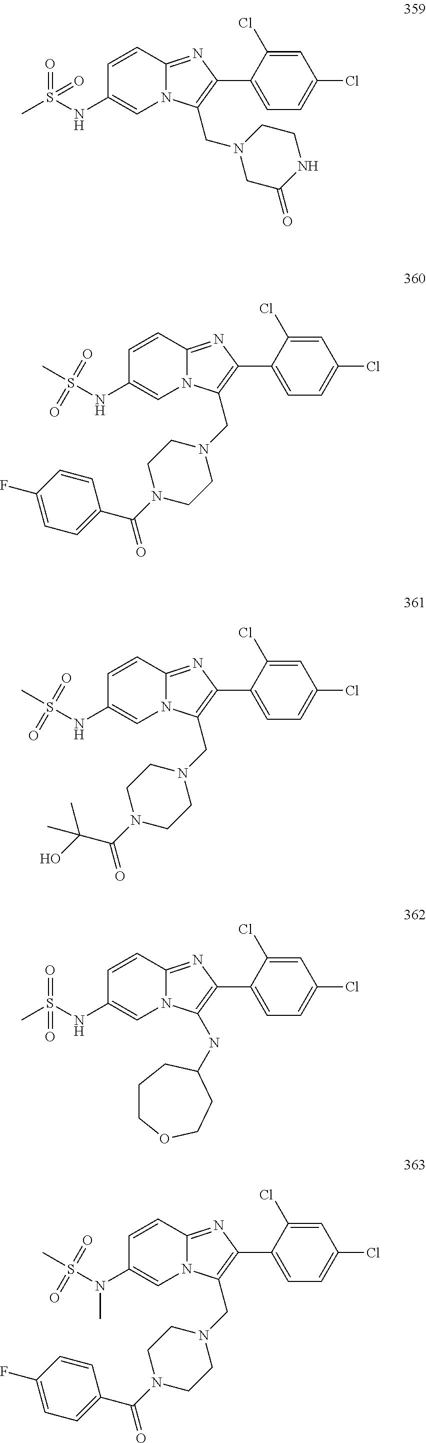 [SMILES: CS(=O)(=O)Nc1ccc2nc(-c3ccc(Cl)cc3Cl)c(CN3CCNC(=O)C3)n2c1 ; CS(=O)(=O)Nc1ccc2nc(-c3ccc(Cl)cc3Cl)c(CN3CCN(C(=O)c4ccc(F)cc4)CC3)n2c1 ; CC(C)(O)C(=O)N1CCN(Cc2c(-c3ccc(Cl)cc3Cl)nc3ccc(NS(C)(=O)=O)cn23)CC1 ; CS(=O)(=O)Nc1ccc2nc(-c3ccc(Cl)cc3Cl)c(NC3CCCOCC3)n2c1 ; CN(c1ccc2nc(-c3ccc(Cl)cc3Cl)c(CN3CCN(C(=O)c4ccc(F)cc4)CC3)n2c1)S(C)(=O)=O]

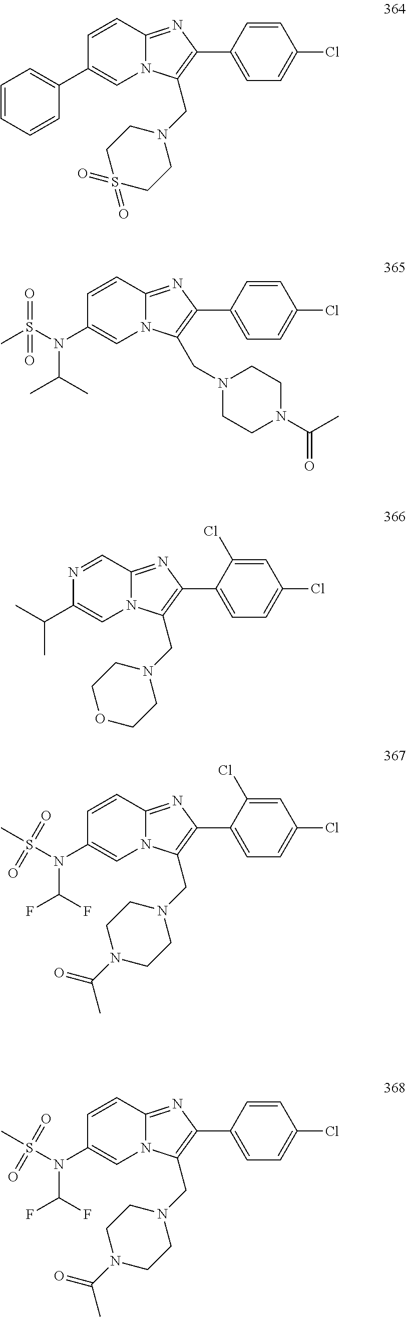

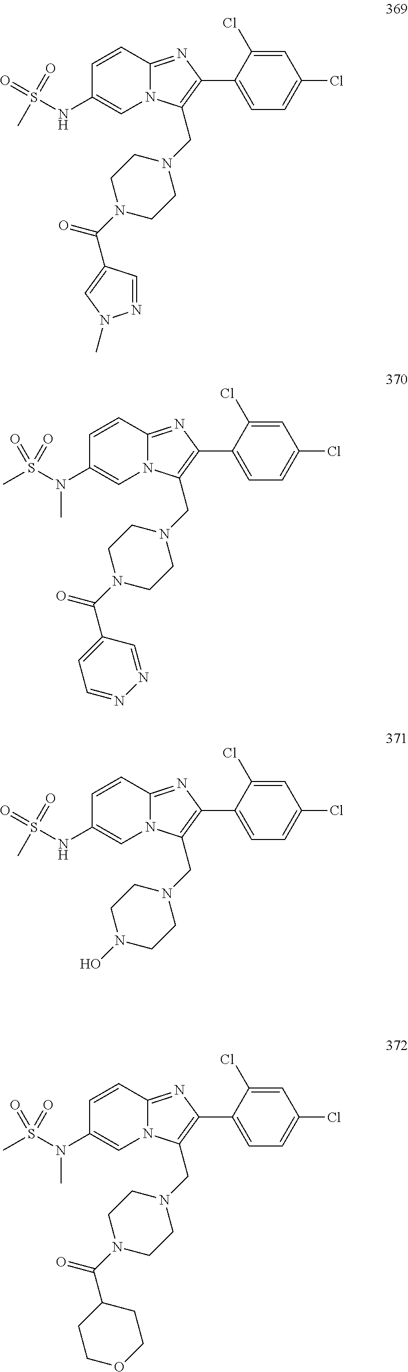 [SMILES: Cn1cc(C(=O)N2CCN(Cc3c(-c4ccc(Cl)cc4Cl)nc4ccc(NS(C)(=O)=O)cn34)CC2)cn1 ; CN(c1ccc2nc(-c3ccc(Cl)cc3Cl)c(CN3CCN(C(=O)c4ccnnc4)CC3)n2c1)S(C)(=O)=O ; CS(=O)(=O)Nc1ccc2nc(-c3ccc(Cl)cc3Cl)c(CN3CCN(O)CC3)n2c1 ; CN(c1ccc2nc(-c3ccc(Cl)cc3Cl)c(CN3CCN(C(=O)C4CCOCC4)CC3)n2c1)S(C)(=O)=O]

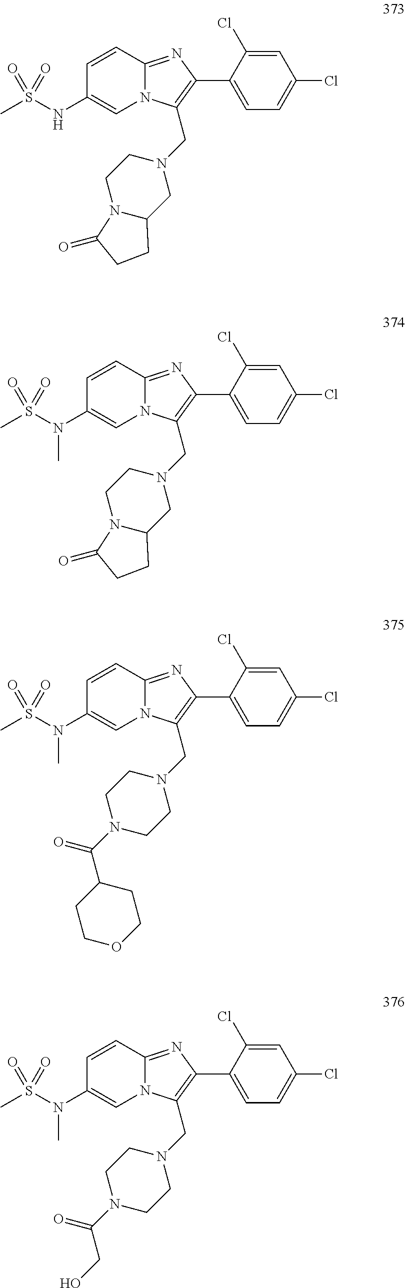 [SMILES: CS(=O)(=O)Nc1ccc2nc(-c3ccc(Cl)cc3Cl)c(CN3CCN4C(=O)CCC4C3)n2c1 ; CN(c1ccc2nc(-c3ccc(Cl)cc3Cl)c(CN3CCN4C(=O)CCC4C3)n2c1)S(C)(=O)=O ; CN(c1ccc2nc(-c3ccc(Cl)cc3Cl)c(CN3CCN(C(=O)C4CCOCC4)CC3)n2c1)S(C)(=O)=O ; CN(c1ccc2nc(-c3ccc(Cl)cc3Cl)c(CN3CCN(C(=O)CO)CC3)n2c1)S(C)(=O)=O]

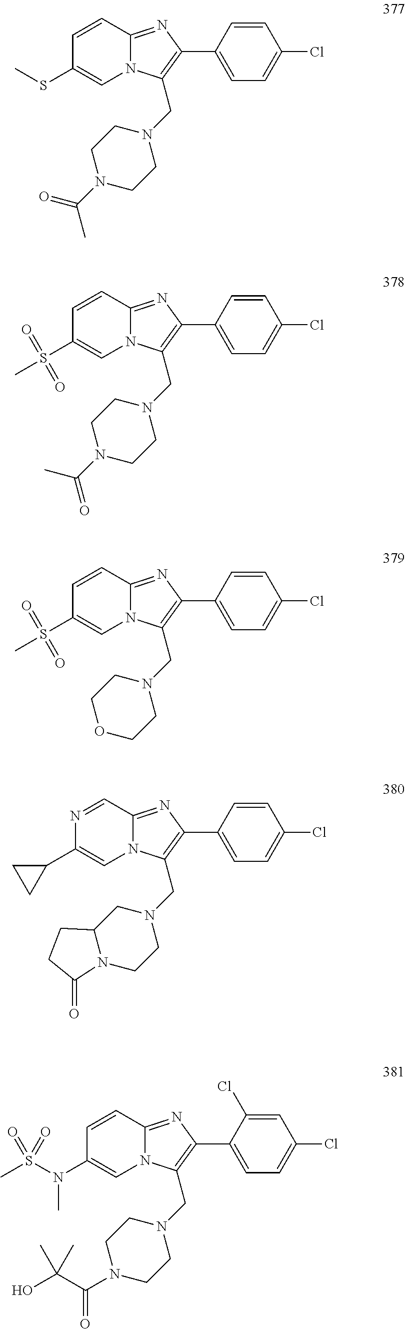 [SMILES: CSc1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(S(C)(=O)=O)cn23)CC1 ; CS(=O)(=O)c1ccc2nc(-c3ccc(Cl)cc3)c(CN3CCOCC3)n2c1 ; O=C1CCC2CN(Cc3c(-c4ccc(Cl)cc4)nc4cnc(C5CC5)cn34)CCN12 ; CN(c1ccc2nc(-c3ccc(Cl)cc3Cl)c(CN3CCN(C(=O)C(C)(C)O)CC3)n2c1)S(C)(=O)=O]

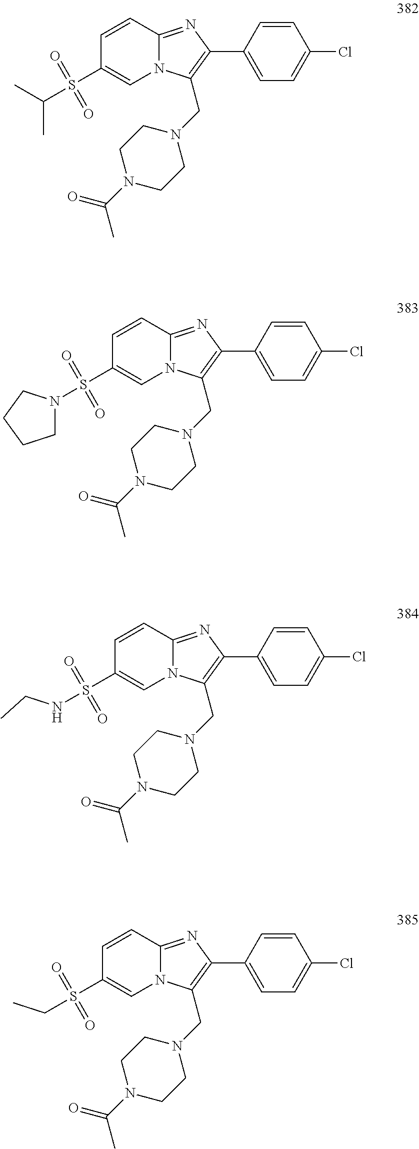 [SMILES: CC(=O)N1CCN(Cc2c(-c3ccc(Cl)cc3)nc3ccc(S(=O)(=O)C(C)C)cn23)CC1 ; CC(=O)N1CCN(Cc2c(-c3ccc(Cl)cc3)nc3ccc(S(=O)(=O)N4CCCC4)cn23)CC1 ; CCNS(=O)(=O)c1ccc2nc(-c3ccc(Cl)cc3)c(CN3CCN(C(C)=O)CC3)n2c1 ; CCS(=O)(=O)c1ccc2nc(-c3ccc(Cl)cc3)c(CN3CCN(C(C)=O)CC3)n2c1]

Typically, the above formulae (and all formulae herein) are shown in non-stereoisomeric form. For the avoidance of doubt, throughout the present disclosure a single formula is intended to represent all possible stereoisomers of a particular structure, including all possible isolated enantiomers corresponding to the formula, all possible mixtures of enantiomers corresponding to the formula, all possible mixtures of diastereomers corresponding to the formula, all possible mixtures of epimers corresponding to the formula and all possible racemic mixtures corresponding to the formula.

Further provided by the invention is a compound for use in treating, or preventing, a cancer, an inflammatory condition, an autoimmune condition, a neurological condition, a proliferative disorder, and/or a metabolic condition, which compound is any compound as defined above. The cancer or condition is not especially limited, provided it is one that may be treated, ameliorated, prevented and/or cured by inhibiting SLC2A class I transporter function, preferably GLUT1, GLUT2, GLUT3, GLUT4 and/or GLUT14 receptor function.

The biological function of SLC2A class I transporters in relation to cancer has been explained in detail above, with reference to the literature. The inventors have determined from this that SLC2A class I transporter inhibitors may have utility against all cancers. Thus, the nature of the cancer is not especially limited. In typical embodiments, the cancer is a cancer selected from a solid or liquid tumour or a cancer wherein basal glucose transport is up-regulated.

These include but are not limited to cancer of the eye, brain (such as gliomas, glioblastomas, medullablastomas, craniopharyngioma, ependymoma, and astrocytoma), spinal cord, kidney, mouth, lip, throat, oral cavity, nasal cavity, small intestine, colon, parathyroid gland, gall bladder, head and neck, breast, bone, bile duct, cervix, heart, hypopharyngeal gland, lung, bronchus, liver, skin, ureter, urethra, testicles, vagina, anus, laryngeal gland, ovary, thyroid, oesophagus, nasopharyngeal gland, pituitary gland, salivary gland, prostate, pancreas, adrenal glands; an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia, juvenile myelomonocytic leukaemia, Hodgkin lymphoma, non-Hodgkin lymphoma, mantle lymphoma, follicular lymphoma, primary effusion lymphoma, AIDS-related lymphoma, Hodgkin lymphoma, diffuse B cell lymphoma, Burkitt lymphoma, and cutaneous T-cell lymphoma).

The biological function of SLC2A class I transporters in relation to inflammatory conditions and/or the autoimmune conditions has been explained in detail above, with reference to the literature. The inventors have determined from this that SLC2A class I transporter inhibitors may have utility against all such conditions. Thus, the nature of the inflammatory condition and/or the autoimmune condition is not especially limited. In further typical embodiments the inflammatory condition and/or the autoimmune condition are conditions relating to immune B cell and/or T cell dysregulation including aberrant activation. In further typical embodiments the immune B cell and/or T cell dysregulation condition is an inflammatory or autoimmune condition selected from gout, idiopathic pulmonary fibrosis, liver fibrosis, liver cirrhosis, polycystic kidney disease, allergic asthma, acute or chronic idiopathic inflammatory arthritis, osteoarthritis, rheumatoid arthritis, psoriasis, chronic dermatosis, myositis, a demyelinating disease, chronic obstructive pulmonary disease, interstitial lung disease, glomerulonephritis, interstitial nephritis, chronic infectious disease (such as chronic active hepatitis), Crohn's disease, ulcerative colitis, plaque formation in atherosclerosis, a degenerative disease of the joints or nervous system, multiple sclerosis, type I and type II diabetes, celiac disease, acute kidney injury, sepsis, acute liver failure, chronic liver failure, chronic kidney failure, pancreatitis, Grave's disease, psoriasis, septicemia, cystic fibrosis, meningitis and acute respiratory distress syndrome, ankylosing spondylitis, Chagas disease, dermatomyositis, endometriosis, Goodpasture's syndrome, Guillain-Barré syndrome, Hashiomoto's disease, hidradenitis suppurativa, Kawasaki disease, idiopathic thrombocytopenic purpura, lupus (such as systemic lupus erythematosus, discoid lupus, drug-induced lupus, neonatal lupus, and subacute cutaneous lupus), neuromyotonia, pemphigus vulgaris, pernicious anemia, psoriasis, polymyositis, relapsing polychondritis, scleroderma, Sjögren's syndrome, stiff person syndrome, temporal arteritis (also known as giant cell arteritis), vasculitis, vitiligo, Wegener's granulomatosis, organ transplant rejection, uveoretinitis, glomerulonephritis, allergic reaction, fibromyalgia, multiple endocrine failure, Schmidt's syndrome, autoimmune uveitis, Addison's disease, adrenalitis, thyroiditis, autoimmune thyroid disease, gastric atrophy, chronic hepatitis, lupoid hepatitis, atherosclerosis, hypoparathyroidism, Dressler's syndrome, autoimmune thrombocytopenia, idiopathic thrombocytopenic purpura, hemolytic anemia, atopic dermatitis, dermatitis herpetiformis, alopecia arcata, pemphigoid, progressive systemic sclerosis, CREST syndrome (calcinosis, Raynaud's phenomenon, esophageal dysmotility, and sclerodactyl), polyarteritis nedosa, atopic rhinitis, sarcoidosis, rheumatic fever, erythema multiforme, Cushing's syndrome, toxic epidermal necrolysis, alveolitis, allergic alveolitis, fibrosing alveolitis, interstitial lung disease, Takayasu's arteritis, polymyalgia rheumatica, schistosomiasis, ascariasis, aspergillosis, Sampter's syndrome, lymphomatoid granulomatosis, Caplan's syndrome, dengue, encephalomyelitis, endocarditis, endomyocardial fibrosis, endophthalmitis, erythema elevatum et diutinum, erythroblastosis fetalis, eosinophilic faciitis, Shulman's syndrome, Felty's syndrome, filariasis, cyclitis, chronic cyclitis, heterochronic cyclitis, Fuch's cyclitis, Henoch-Schonlein purpura, Eaton-Lambert syndrome, cryoglobulinemia, Waldenstrom's macroglobulemia, and Evan's syndrome.

The biological function of SLC2A class I transporters in relation to proliferative disorders has been explained in detail above, with reference to the literature. The inventors have determined from this that SLC2A class I transporter inhibitors may have utility against all such disorders. Thus, the nature of the proliferative disorder is not especially limited. In yet further typical embodiments the proliferative disorder is a proliferative disorder or condition selected from diseases of benign, pre-malignant, and malignant cellular proliferation, including but not limited to, neoplasms and tumours (such as histocytoma, glioma, astrocyoma, and osteoma), cancers, leukemias, psoriasis, bone diseases, fibroproliferative disorders (such as those of connective tissues), idiopathic pulmonary fibrosis, polycystic kidney disease, renal cyst formation, intimal hyperplasia, chronic liver disease, liver fibrosis, liver cirrhosis, scleroderma, restenosis and atherosclerosis.

The biological function of SLC2A class I transporters in relation to neurological conditions has been explained in detail above, with reference to the literature. The inventors have determined from this that SLC2A class I transporter inhibitors may have utility against all such conditions. Thus, the nature of the neurological condition is not especially limited. In still further typical embodiments the neurological condition is an condition selected from epilepsy, schizophrenia, Alzheimer's disease, Parkinson's disease, Huntington's disease, amyotrophic lateral sclerosis (Lou Gehrig's Disease), frontotemporal dementia, Lewy body dementia, vascular dementia, progressive supranuclear palsy, corticobasal degeneration and multiple system atrophy.

The biological function of SLC2A class I transporters in relation to metabolic conditions has been explained in detail above, with reference to the literature. The inventors have determined from this that SLC2A class I transporter inhibitors may have utility against all such conditions. Thus, the nature of the metabolic condition is not especially limited. In still further typical embodiments the metabolic condition is a metabolic condition selected from metabolic syndrome, obesity, diabetes (such as diabetes type I, diabetes type II, MODY, and gestational diabetes), pre-diabetes, lipodystrophy, impaired glucose tolerance, elevated plasma insulin concentrations, insulin resistance, dyslipidemia, hyperglycemia, hyperlipidemia, hypertriglyceridemia, hypertension, cardiovascular disease or respiratory conditions, hyperphagia, hypophagia, triglyceride storage disease, Bardet-Biedl syndrome, Lawrence-Moon syndrome, Prader-Labhart-Willi syndrome, Kearns-Sayre syndrome, medium chain acyl-CoA dehydrogenase deficiency and cachexia.

The invention also provides a pharmaceutical composition comprising any one or more of the compounds as defined above. The pharmaceutical composition is not especially limited, but typically the pharmaceutical composition comprises a pharmaceutically acceptable additive and/or excipient. Any additive or excipient known in the art for use in pharmaceutical compositions may be employed, provided that it does not interfere detrimentally with the function of the active ingredient. The pharmaceutical composition is typically for treating, preventing, ameliorating, controlling and/or curing a cancer or a condition or a disorder as defined above. In some embodiments, and preferably when the pharmaceutical composition is for treating a cancer, it may further comprise a second active ingredient (such as a second (further) agent for treating cancer). Such embodiments are preferred in cancers, conditions, disorders and/or patients who may benefit from combination therapies.

In such combination pharmaceutical compositions, the further agent for treating cancer may be selected from anti-microtubule agents, platinum coordination complexes, alkylating agents, antibiotic agents, topoisomerase II inhibitors, antimetabolites, topoisomerase I inhibitors, hormones and hormone analogues, signal transduction pathway inhibitors, non-receptor tyrosine kinase angiogenesis inhibitors, immunotherapeutic agents, proapoptotic agents and cell cycle signalling inhibitors.

The invention further provides a method of treating a cancer and/or a condition and/or a disorder, which method comprises administering to a patient any compound or composition as defined in above. Typically the cancer, condition, or disorder is a cancer, condition, or disorder as defined above. The patient is not especially limited, and may be an animal (preferably a mammal) or a human, but preferably the patient is human. When the treatment or pharmaceutical composition is a combination treatment, the method of treatment may comprise administering to a patient a compound or a composition of the invention and a further agent for treating a cancer condition or disorder as defined above.

When the treatment or pharmaceutical composition is a combination treatment, the compound or composition and the further agent may be packaged for administration simultaneously, sequentially or separately. In such embodiments, the method of treatment may comprise administering to a patient the compound or composition of the invention and the further agent simultaneously, sequentially or separately.

The invention further provides a method of synthesis of a compound as defined above, which method comprises reacting a substituted or unsubstituted pyridine compound with a substituted or unsubstituted ketone compound in a ring forming step. The method is not especially limited, provided that it is capable of producing at least one of the above compounds.

In a typical embodiment, the method comprises a ring-forming step as follows:

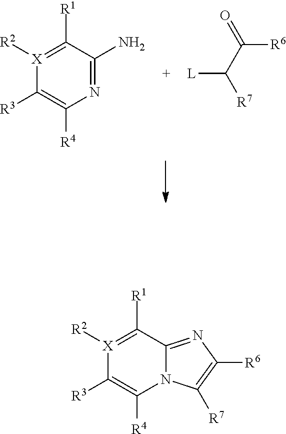

wherein L is a leaving group; X is C or N; and wherein R¹, R², R³, R⁴, R⁶ and R⁷ are independently selected from H or an organic group; and wherein when X is NR² is absent; optionally wherein when any of R¹, R², R³, R⁴, R⁶ and R⁷ is H, the method comprises a further step of substituting that H with an organic group; preferably wherein the R¹, R², R³, R⁴, R⁶ and R⁷ groups are substituents as defined above.

The L group is not especially limited, and may be any leaving group known in organic chemistry, provided that it does not detrimentally interfere with the method. The L group may be selected by the skilled person with reference to known synthesis techniques. Typically the L is selected from the following groups.

A halogen (such as F, Cl, Br, and I).

An —OH and an alkoxy group (such as —OMe, —OEt, —OPr, and —OPh).

One of the following groups: —N₂⁺, OR′₂⁺, —OSO₂C₄F₉, —OSO₂CF₃, —OSO₂F, a tosylate (—OTs), a mesylate (—OMs), —OH₂⁺, an acyl halide (such as —CO—F, —CO—Cl, —CO—Br, —CO—I), —OH(C₁-C₆ alkyl)⁺, —ONO₂, —OPO(OH)₂, an inorganic ester, —S(C₁-C₆ alkyl)₂⁺, —N(C₁-C₆ alkyl)₃⁺, —OCO(C₁-C₆ alkyl), and —NH₃⁺.

In the present method, typically the ring-forming step is carried out by refluxing under acid or base catalysis. The skilled person may select the type and strength of acid or base, and the reaction conditions, with reference to known synthesis techniques.

In more typical embodiments, the method comprises the following steps:

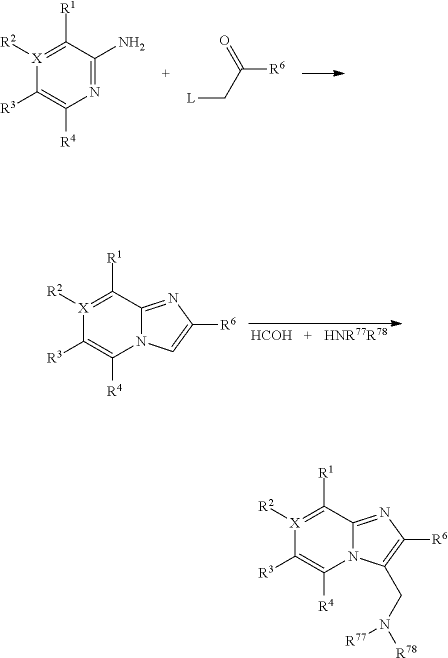

wherein R⁷⁷ and R⁷⁸ may alone or together form any of the compounds as defined above.

In these typical embodiments, the HNR⁷⁷R⁷⁸ reactant is selected from a substituted or unsubstituted pyrrolidine compound, a substituted or unsubstituted piperidine compound, a substituted or unsubstituted piperazine compound, and a substituted or unsubstituted morpholine compound. The substituents, if present, may be any of the substituents already defined above.

In addition to compounds for use in medicine, the present invention, and in particular the synthetic method, provides compounds that were not previously known, such compounds comprising a formula selected from one of the following:

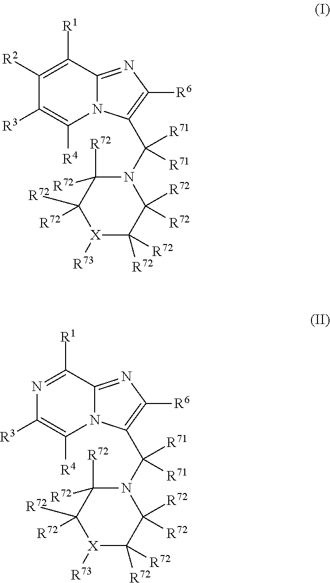

wherein in (I) the substituents may be selected from one of the following groups (i)-(vi):
(i) R³ is selected from a substituted or unsubstituted saturated carbocyclic ring having from 3 to 7 ring carbons; and a substituted or unsubstituted aromatic ring having from 3 to 7 ring carbons provided that it is not a 4-methylphenyl group; R⁶ is a substituted or unsubstituted saturated or unsaturated heterocyclic group having from 4 to 7 ring atoms; X is N; R⁷³ is a —(CO)—R⁷⁵ group; and wherein R¹, R², R⁴, R⁷¹, R⁷² and R⁷⁵ are independently selected from H and a substituted or unsubstituted organic group; preferably wherein R¹, R², R⁴, R⁷¹, R⁷² and R⁷⁵ are as defined above;
(ii) R³ is a substituted or unsubstituted saturated or unsaturated heterocyclic group having from 4 to 7 ring atoms; R⁶ is a substituted or unsubstituted saturated or unsaturated heterocyclic group having from 4 to 7 ring atoms; X is independently selected from N, O and S wherein R⁷³ is absent when its X is O or S; and wherein R¹, R², R⁴, R⁷¹, R⁷² and R⁷³ are independently selected from H and a substituted or unsubstituted organic group; preferably wherein R¹, R², R⁴, R⁷¹, R⁷² and R⁷³ are as defined above;
(iii) R³ is a —Z(R³³)₂ group in which Z can be C or N wherein a further H or substituted or unsubstituted organic group or R³³ group may be present when Z is C; R³³ is selected from a substituted or unsubstituted linear or branched alkyl group optionally forming a non-aromatic carbocyclic or heterocyclic ring with another R³³, a substituted sulphonyl group, and a substituted carbonyl group; provided that the —Z(R³³)₂ group is not —NH₂ or an amide bonded to the ring via a C atom; R⁶ is a substituted or unsubstituted saturated or unsaturated heterocyclic group preferably having from 4 to 7 ring atoms; X is N; and wherein R⁷³ is a —(CO)—R⁷⁵ group; and wherein R¹, R², R⁴, R⁷¹, R⁷² and R⁷⁵ are independently selected from H and a substituted or unsubstituted organic group; preferably wherein R¹, R², R⁴, R⁷¹, R⁷² and R⁷⁵ are as defined above;
(iv) R⁶ is a substituted or unsubstituted saturated homocyclic or heterocyclic group having from 3-7 ring atoms in which each ring atom is selected from C, N, O or S, provided that R⁶ is not adamantyl; X is independently selected from N, O and S wherein R⁷³ is absent when its X is O or S; and wherein R¹, R², R³, R⁴, R⁷¹, R⁷² and R⁷³ are independently selected from H and a substituted or unsubstituted organic group; preferably wherein R¹, R², R³, R⁴, R⁷¹, R⁷² and R⁷³ are as defined above;
(v) groups such that the compound has a structure as follows:

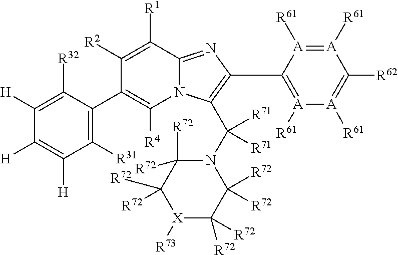

wherein R¹, R², R⁴, R⁷¹, R⁷² and R⁷³ are independently selected from H and a substituted or unsubstituted organic group; adjacent R groups may together form a substituted or unsubstituted saturated or unsaturated aliphatic or aromatic homocyclic or heterocyclic ring; A may independently be C or N wherein R⁶¹ is absent when A is N; X is independently selected from N, O and S wherein R⁷³ is absent when its X is O or S; R³¹ is selected from H and a substituted or unsubstituted organic group; R³² is selected from a substituted or unsubstituted organic group excluding H; R⁶¹ is selected from H and a substituted or unsubstituted organic group; and R⁶² is selected from a substituted or unsubstituted organic group excluding H and Me; and
(vi) groups such that the compound has a structure as follows:

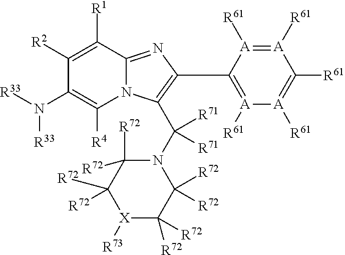

wherein R¹, R², R⁴, R⁷¹, R⁷² and R⁷³ are independently selected from H and a substituted or unsubstituted organic group; adjacent R groups may together form a substituted or unsubstituted saturated or unsaturated aliphatic or aromatic homocyclic or heterocyclic ring; each A may independently be C or N wherein R⁶¹ is absent when A is N; X is independently selected from N, O and S wherein R⁷³ is absent when its X is O or S; Z can be C or N wherein a further H or substituted or unsubstituted organic group or R³³ group may be present when Z is C; R³³ is selected from a substituted or unsubstituted linear or branched alkyl group optionally forming a non-aromatic carbocyclic or heterocyclic ring with another R³³, a substituted sulphonyl group, and a substituted carbonyl group; and R⁶¹ is selected from H and a substituted or unsubstituted organic group;

and wherein in (II) the substituents are as follows:

R¹, R³ R⁴, R⁷¹, R⁷² and R⁷³ are independently selected from H and a substituted or unsubstituted organic group, provided that R³ is not Me; adjacent R groups may together form a substituted or unsubstituted saturated or unsaturated aliphatic or aromatic homocyclic or heterocyclic ring; X is independently selected from N, O and S wherein R⁷³ is absent when its X is O or S; and R⁶ is selected from H and a substituted or unsubstituted organic group except CO₂H and CO₂Et; preferably wherein R¹, R³, R⁴, R⁷¹, R⁷² and R⁷³ are as defined above.

In typical embodiments, these compounds are compounds wherein R¹, R², R³, R⁴, R⁶, R⁷¹, R⁷², R⁷³ and R⁷⁵ are as defined above.

In more typical embodiments, the compound is a compound of any of the following formulae:

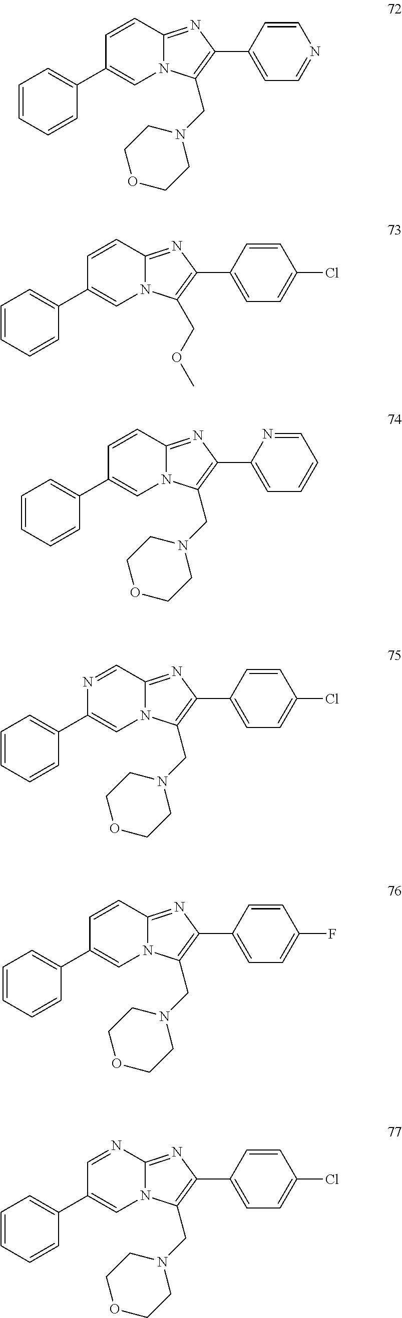 [SMILES: c1ccc(-c2ccc3nc(-c4ccncc4)c(CN4CCOCC4)n3c2)cc1 ; COCc1c(-c2ccc(Cl)cc2)nc2ccc(-c3ccccc3)cn12 ; c1ccc(-c2ccc3nc(-c4ccccn4)c(CN4CCOCC4)n3c2)cc1 ; Clc1ccc(-c2nc3cnc(-c4ccccc4)cn3c2CN2CCOCC2)cc1 ; Fc1ccc(-c2nc3ccc(-c4ccccc4)cn3c2CN2CCOCC2)cc1 ; Clc1ccc(-c2nc3ncc(-c4ccccc4)cn3c2CN2CCOCC2)cc1]

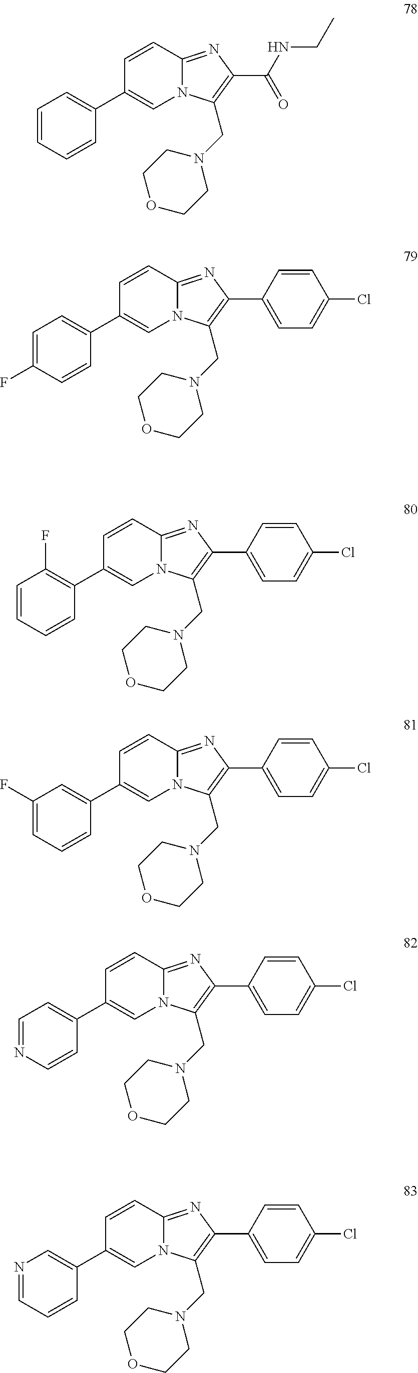

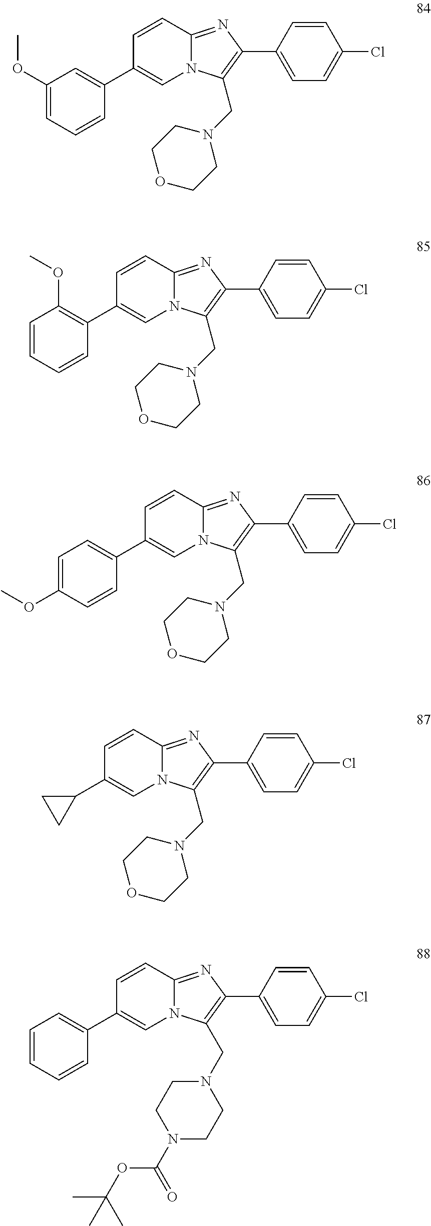 [SMILES: COc1cccc(-c2ccc3nc(-c4ccc(Cl)cc4)c(CN4CCOCC4)n3c2)c1 ; COc1ccccc1-c1ccc2nc(-c3ccc(Cl)cc3)c(CN3CCOCC3)n2c1 ; COc1ccc(-c2ccc3nc(-c4ccc(Cl)cc4)c(CN4CCOCC4)n3c2)cc1 ; Clc1ccc(-c2nc3ccc(C4CC4)cn3c2CN2CCOCC2)cc1 ; CC(C)(C)OC(=O)N1CCN(Cc2c(-c3ccc(Cl)cc3)nc3ccc(-c4ccccc4)cn23)CC1]

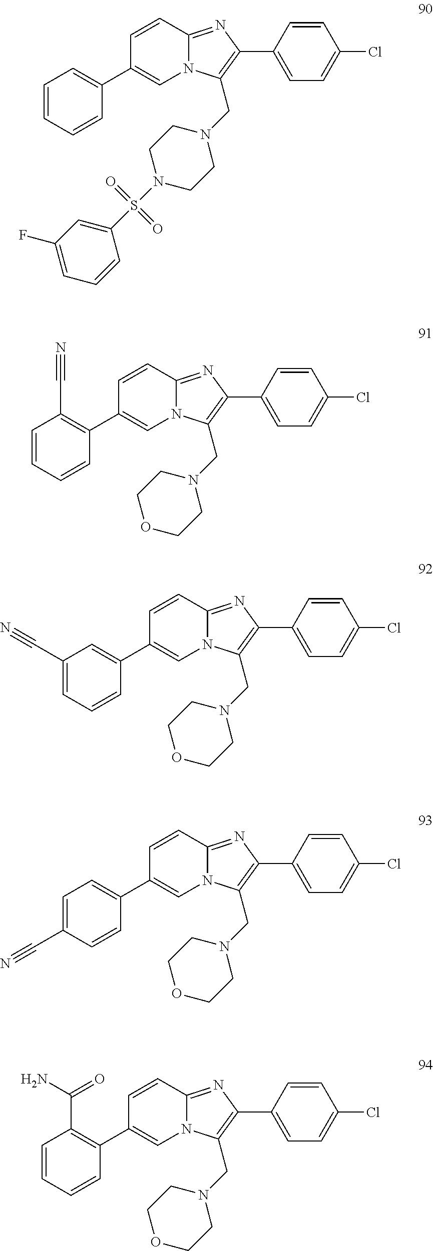 [SMILES: O=S(=O)(c1cccc(F)c1)N1CCN(Cc2c(-c3ccc(Cl)cc3)nc3ccc(-c4ccccc4)cn23)CC1 ; N#Cc1ccccc1-c1ccc2nc(-c3ccc(Cl)cc3)c(CN3CCOCC3)n2c1 ; N#Cc1cccc(-c2ccc3nc(-c4ccc(Cl)cc4)c(CN4CCOCC4)n3c2)c1 ; N#Cc1ccc(-c2ccc3nc(-c4ccc(Cl)cc4)c(CN4CCOCC4)n3c2)cc1 ; NC(=O)c1ccccc1-c1ccc2nc(-c3ccc(Cl)cc3)c(CN3CCOCC3)n2c1]

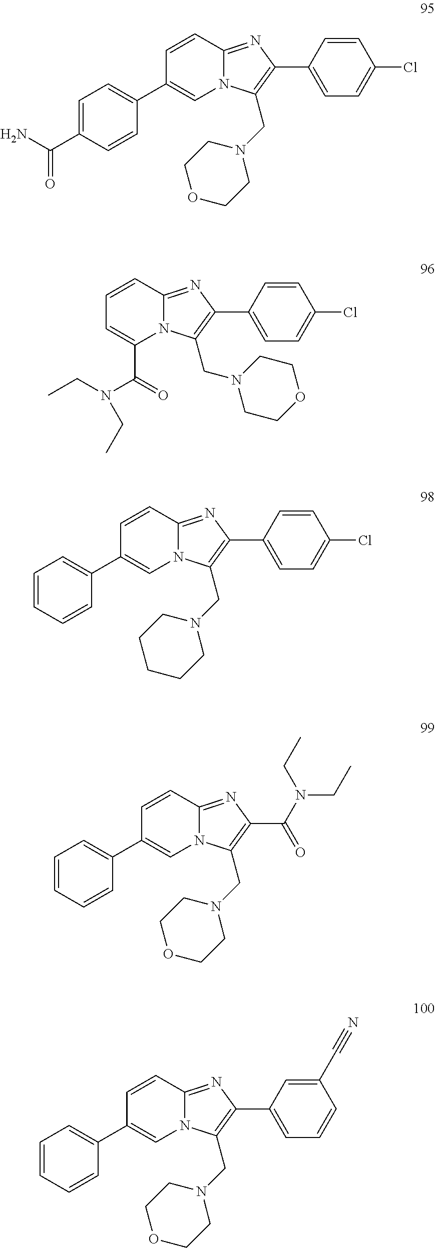 [SMILES: NC(=O)c1ccc(-c2ccc3nc(-c4ccc(Cl)cc4)c(CN4CCOCC4)n3c2)cc1 ; CCN(CC)C(=O)c1cccc2nc(-c3ccc(Cl)cc3)c(CN3CCOCC3)n12 ; Clc1ccc(-c2nc3ccc(-c4ccccc4)cn3c2CN2CCCCC2)cc1 ; CCN(CC)C(=O)c1nc2ccc(-c3ccccc3)cn2c1CN1CCOCC1 ; N#Cc1cccc(-c2nc3ccc(-c4ccccc4)cn3c2CN2CCOCC2)c1]

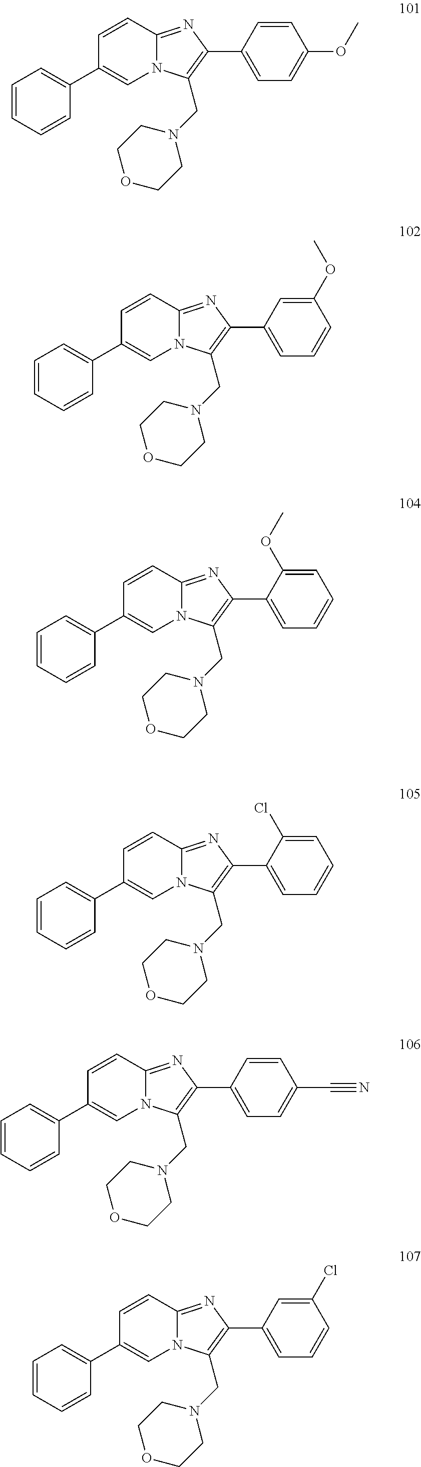 [SMILES: COc1ccc(-c2nc3ccc(-c4ccccc4)cn3c2CN2CCOCC2)cc1 ; COc1cccc(-c2nc3ccc(-c4ccccc4)cn3c2CN2CCOCC2)c1 ; COc1ccccc1-c1nc2ccc(-c3ccccc3)cn2c1CN1CCOCC1 ; Clc1ccccc1-c1nc2ccc(-c3ccccc3)cn2c1CN1CCOCC1 ; N#Cc1ccc(-c2nc3ccc(-c4ccccc4)cn3c2CN2CCOCC2)cc1 ; Clc1cccc(-c2nc3ccc(-c4ccccc4)cn3c2CN2CCOCC2)c1]

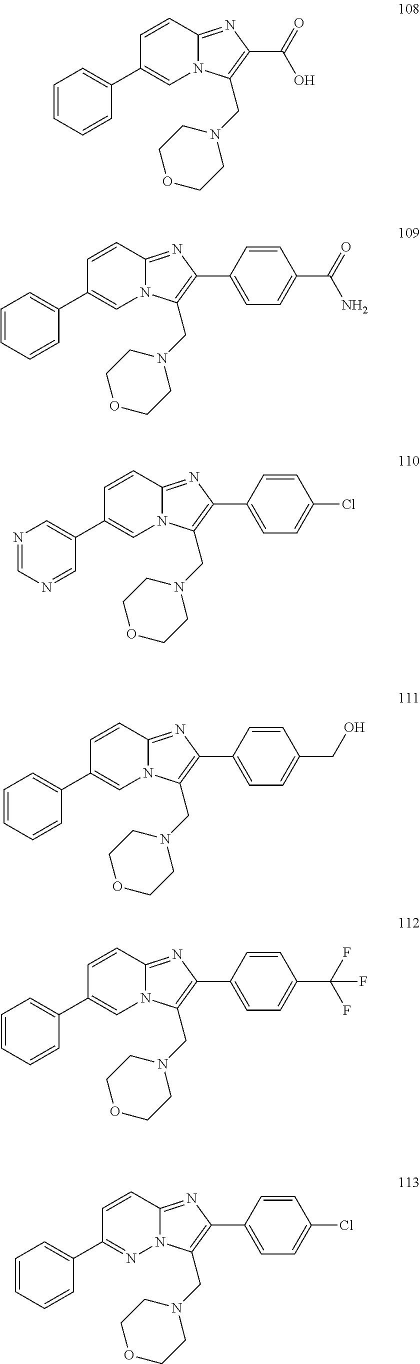 [SMILES: O=C(O)c1nc2ccc(-c3ccccc3)cn2c1CN1CCOCC1 ; NC(=O)c1ccc(-c2nc3ccc(-c4ccccc4)cn3c2CN2CCOCC2)cc1 ; Clc1ccc(-c2nc3ccc(-c4cncnc4)cn3c2CN2CCOCC2)cc1 ; OCc1ccc(-c2nc3ccc(-c4ccccc4)cn3c2CN2CCOCC2)cc1 ; FC(F)(F)c1ccc(-c2nc3ccc(-c4ccccc4)cn3c2CN2CCOCC2)cc1 ; Clc1ccc(-c2nc3ccc(-c4ccccc4)nn3c2CN2CCOCC2)cc1]

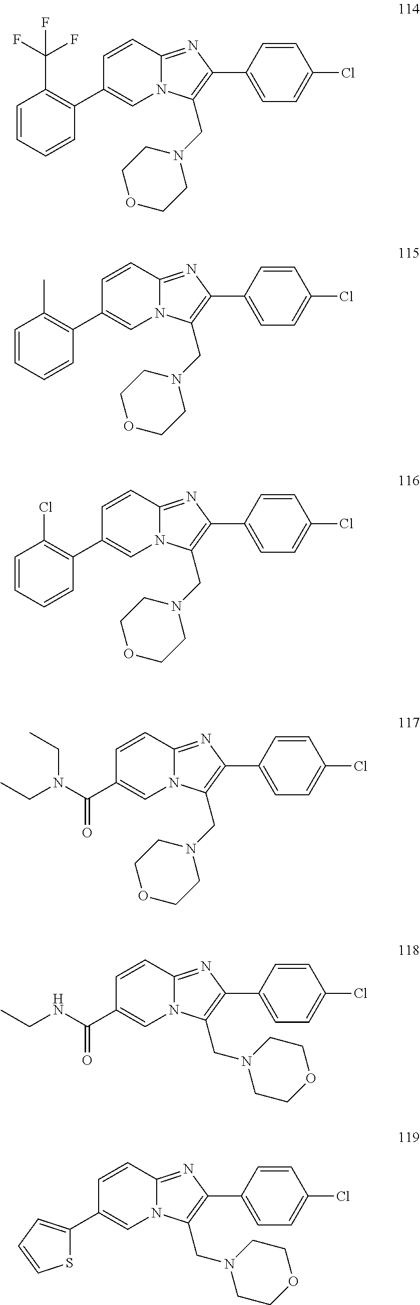 [SMILES: FC(F)(F)c1ccccc1-c1ccc2nc(-c3ccc(Cl)cc3)c(CN3CCOCC3)n2c1 ; Cc1ccccc1-c1ccc2nc(-c3ccc(Cl)cc3)c(CN3CCOCC3)n2c1 ; Clc1ccc(-c2nc3ccc(-c4ccccc4Cl)cn3c2CN2CCOCC2)cc1 ; CCN(CC)C(=O)c1ccc2nc(-c3ccc(Cl)cc3)c(CN3CCOCC3)n2c1 ; CCNC(=O)c1ccc2nc(-c3ccc(Cl)cc3)c(CN3CCOCC3)n2c1 ; Clc1ccc(-c2nc3ccc(-c4cccs4)cn3c2CN2CCOCC2)cc1]

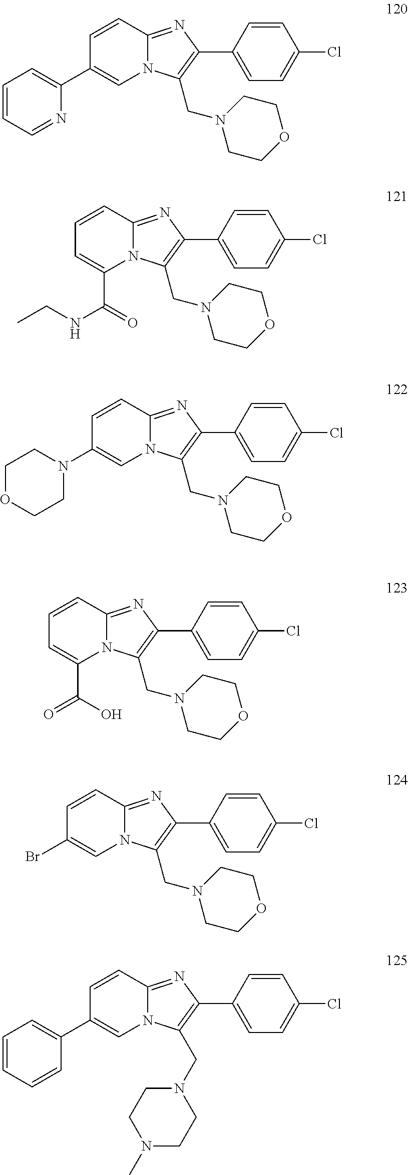

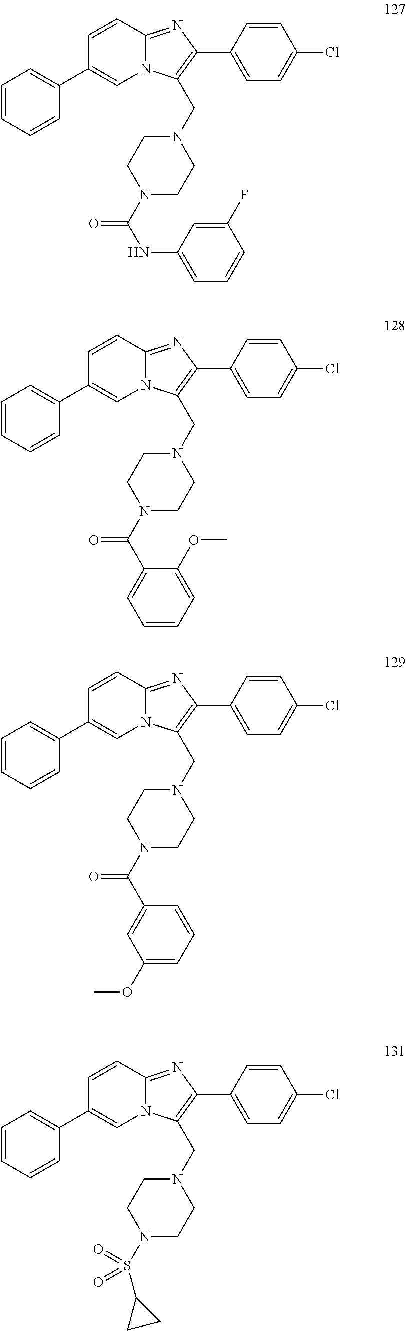 [SMILES: O=C(Nc1cccc(F)c1)N1CCN(Cc2c(-c3ccc(Cl)cc3)nc3ccc(-c4ccccc4)cn23)CC1 ; COc1ccccc1C(=O)N1CCN(Cc2c(-c3ccc(Cl)cc3)nc3ccc(-c4ccccc4)cn23)CC1 ; COc1cccc(C(=O)N2CCN(Cc3c(-c4ccc(Cl)cc4)nc4ccc(-c5ccccc5)cn34)CC2)c1 ; O=S(=O)(C1CC1)N1CCN(Cc2c(-c3ccc(Cl)cc3)nc3ccc(-c4ccccc4)cn23)CC1]

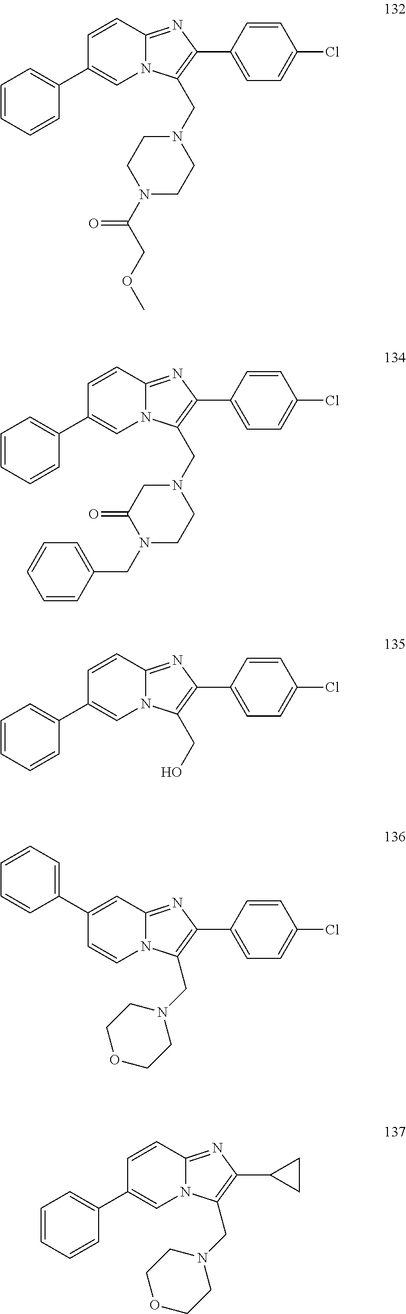 [SMILES: COCC(=O)N1CCN(Cc2c(-c3ccc(Cl)cc3)nc3ccc(-c4ccccc4)cn23)CC1 ; O=C1CN(Cc2c(-c3ccc(Cl)cc3)nc3ccc(-c4ccccc4)cn23)CCN1Cc1ccccc1 ; OCc1c(-c2ccc(Cl)cc2)nc2ccc(-c3ccccc3)cn12 ; Clc1ccc(-c2nc3cc(-c4ccccc4)ccn3c2CN2CCOCC2)cc1 ; c1ccc(-c2ccc3nc(C4CC4)c(CN4CCOCC4)n3c2)cc1]

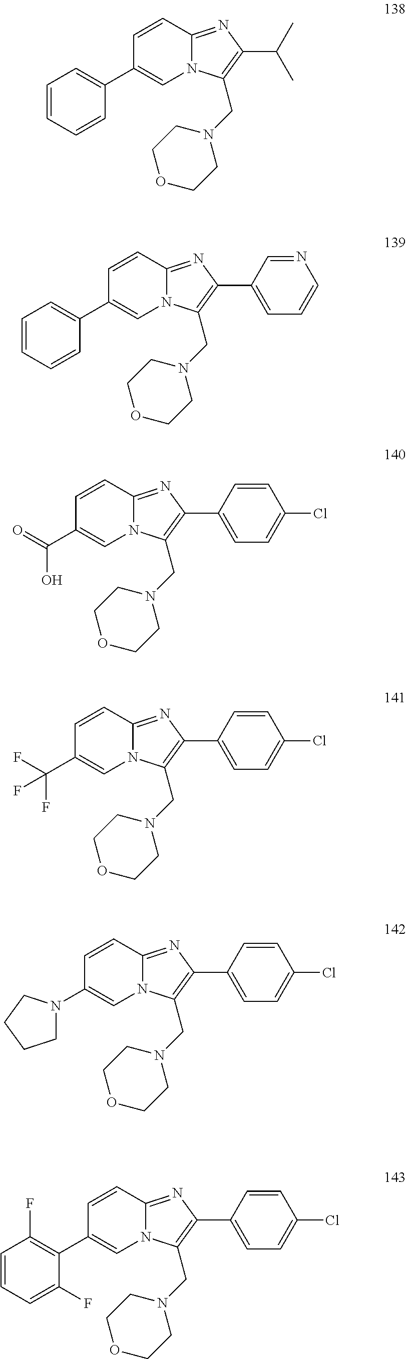 [SMILES: CC(C)c1nc2ccc(-c3ccccc3)cn2c1CN1CCOCC1 ; c1ccc(-c2ccc3nc(-c4cccnc4)c(CN4CCOCC4)n3c2)cc1 ; O=C(O)c1ccc2nc(-c3ccc(Cl)cc3)c(CN3CCOCC3)n2c1 ; FC(F)(F)c1ccc2nc(-c3ccc(Cl)cc3)c(CN3CCOCC3)n2c1 ; Clc1ccc(-c2nc3ccc(N4CCCC4)cn3c2CN2CCOCC2)cc1 ; Fc1cccc(F)c1-c1ccc2nc(-c3ccc(Cl)cc3)c(CN3CCOCC3)n2c1]

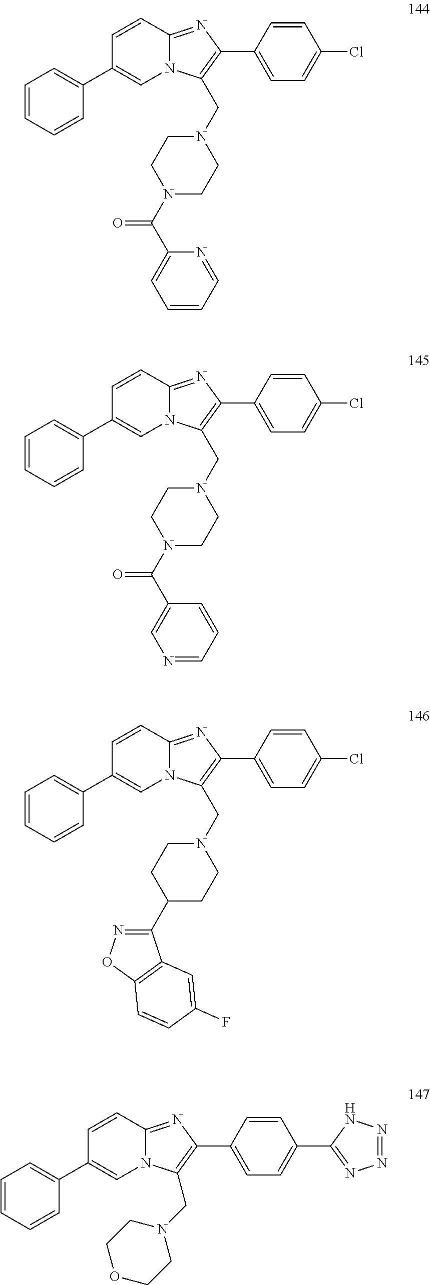 [SMILES: O=C(c1ccccn1)N1CCN(Cc2c(-c3ccc(Cl)cc3)nc3ccc(-c4ccccc4)cn23)CC1 ; O=C(c1cccnc1)N1CCN(Cc2c(-c3ccc(Cl)cc3)nc3ccc(-c4ccccc4)cn23)CC1 ; Fc1ccc2onc(C3CCN(Cc4c(-c5ccc(Cl)cc5)nc5ccc(-c6ccccc6)cn45)CC3)c2c1 ; c1ccc(-c2ccc3nc(-c4ccc(-c5nnn[nH]5)cc4)c(CN4CCOCC4)n3c2)cc1]

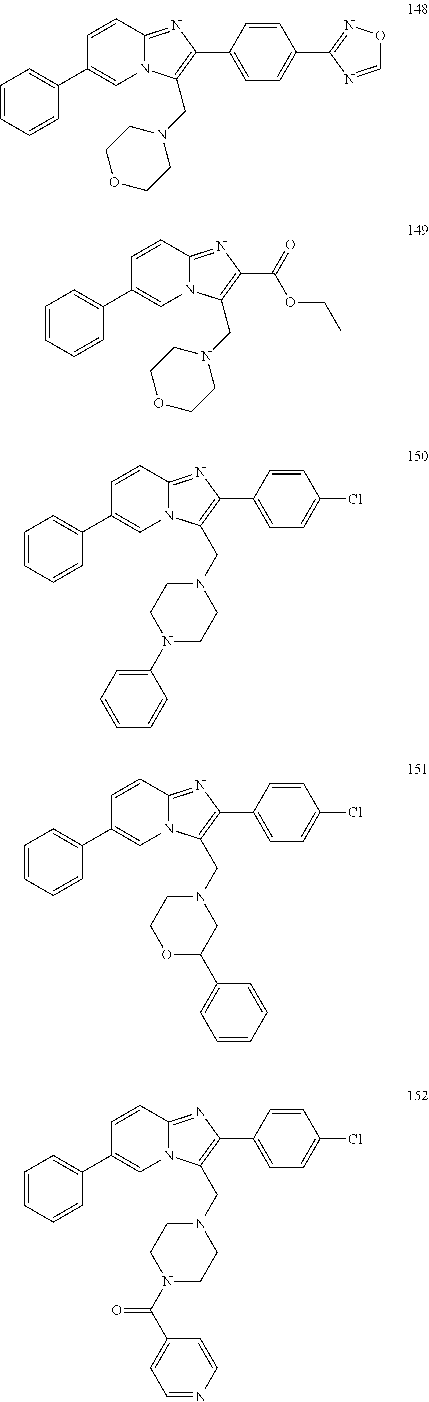 [SMILES: c1ccc(-c2ccc3nc(-c4ccc(-c5ncon5)cc4)c(CN4CCOCC4)n3c2)cc1 ; CCOC(=O)c1nc2ccc(-c3ccccc3)cn2c1CN1CCOCC1 ; Clc1ccc(-c2nc3ccc(-c4ccccc4)cn3c2CN2CCN(c3ccccc3)CC2)cc1 ; Clc1ccc(-c2nc3ccc(-c4ccccc4)cn3c2CN2CCOC(c3ccccc3)C2)cc1 ; O=C(c1ccncc1)N1CCN(Cc2c(-c3ccc(Cl)cc3)nc3ccc(-c4ccccc4)cn23)CC1]

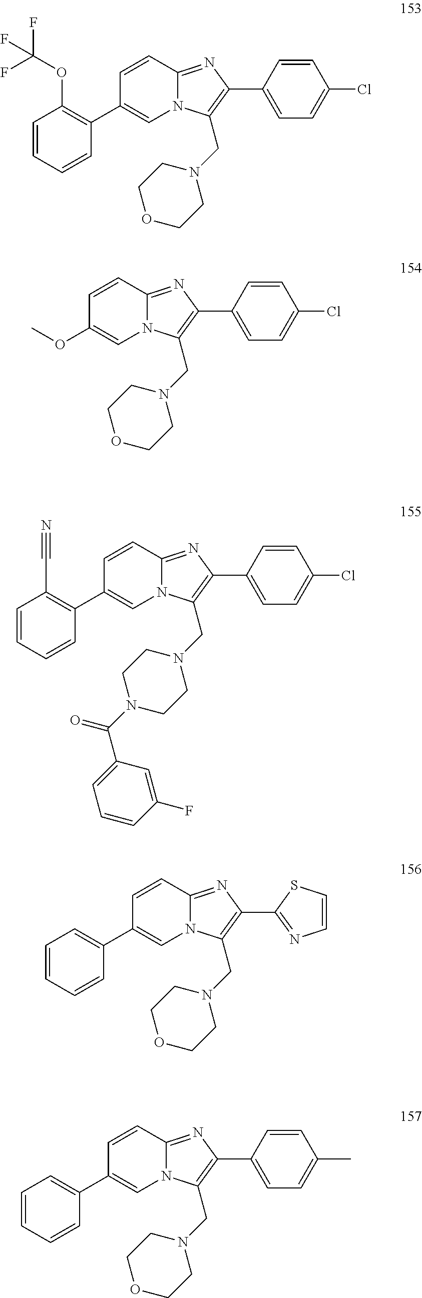 [SMILES: FC(F)(F)Oc1ccccc1-c1ccc2nc(-c3ccc(Cl)cc3)c(CN3CCOCC3)n2c1 ; COc1ccc2nc(-c3ccc(Cl)cc3)c(CN3CCOCC3)n2c1 ; N#Cc1ccccc1-c1ccc2nc(-c3ccc(Cl)cc3)c(CN3CCN(C(=O)c4cccc(F)c4)CC3)n2c1 ; c1ccc(-c2ccc3nc(-c4nccs4)c(CN4CCOCC4)n3c2)cc1 ; Cc1ccc(-c2nc3ccc(-c4ccccc4)cn3c2CN2CCOCC2)cc1]

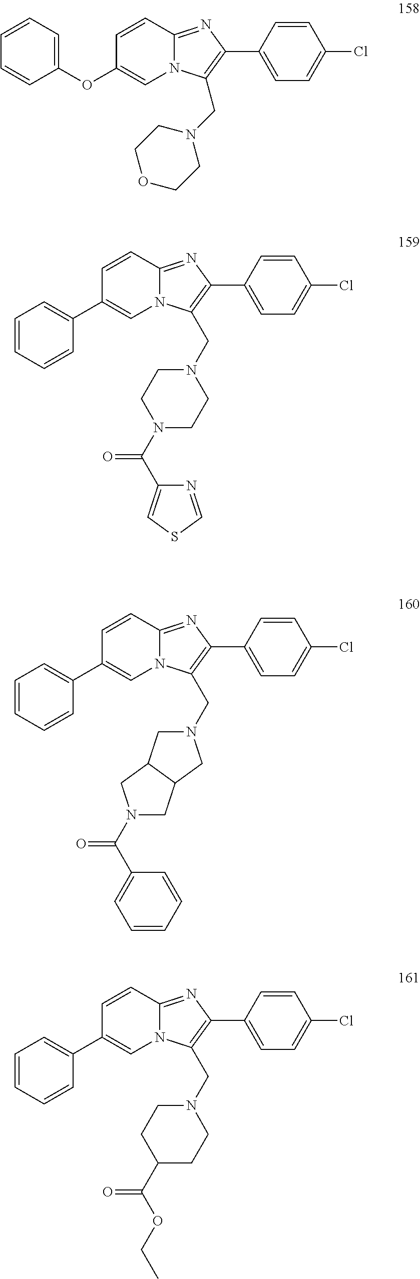 [SMILES: Clc1ccc(-c2nc3ccc(Oc4ccccc4)cn3c2CN2CCOCC2)cc1 ; O=C(c1cscn1)N1CCN(Cc2c(-c3ccc(Cl)cc3)nc3ccc(-c4ccccc4)cn23)CC1 ; O=C(c1ccccc1)N1CC2CN(Cc3c(-c4ccc(Cl)cc4)nc4ccc(-c5ccccc5)cn34)CC2C1 ; CCOC(=O)C1CCN(Cc2c(-c3ccc(Cl)cc3)nc3ccc(-c4ccccc4)cn23)CC1]

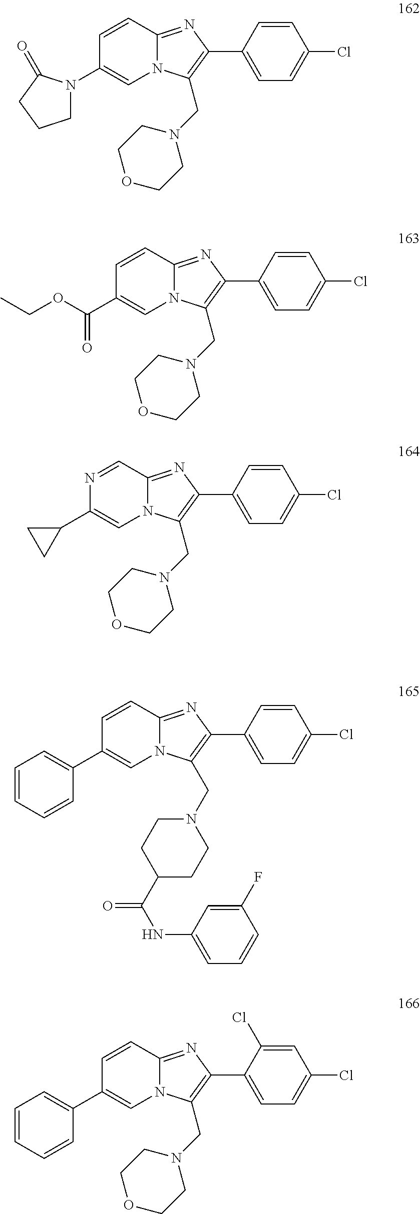 [SMILES: O=C1CCCN1c1ccc2nc(-c3ccc(Cl)cc3)c(CN3CCOCC3)n2c1 ; CCOC(=O)c1ccc2nc(-c3ccc(Cl)cc3)c(CN3CCOCC3)n2c1 ; Clc1ccc(-c2nc3cnc(C4CC4)cn3c2CN2CCOCC2)cc1 ; O=C(Nc1cccc(F)c1)C1CCN(Cc2c(-c3ccc(Cl)cc3)nc3ccc(-c4ccccc4)cn23)CC1 ; Clc1ccc(-c2nc3ccc(-c4ccccc4)cn3c2CN2CCOCC2)c(Cl)c1]

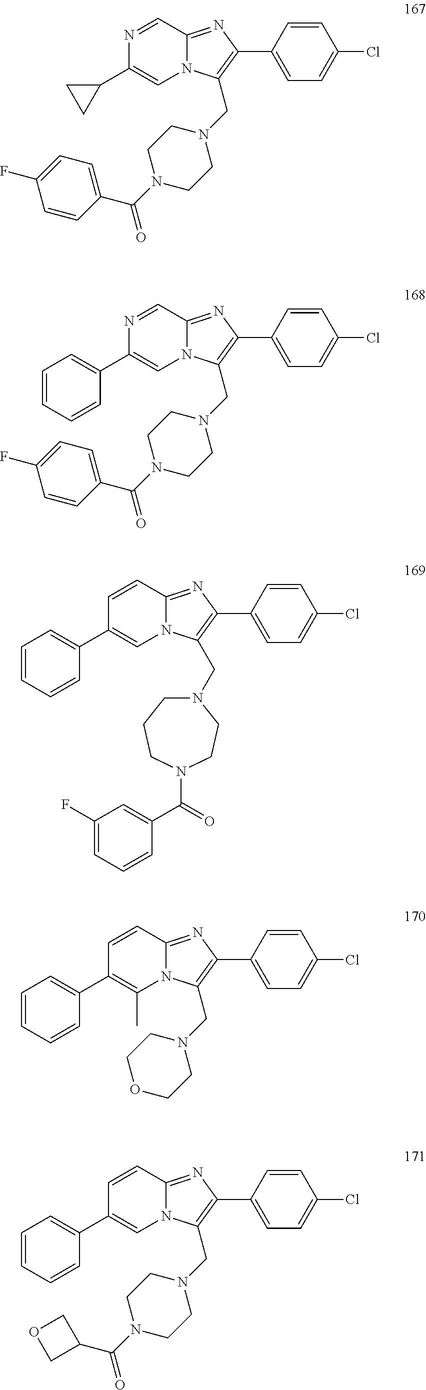 [SMILES: O=C(c1ccc(F)cc1)N1CCN(Cc2c(-c3ccc(Cl)cc3)nc3cnc(C4CC4)cn23)CC1 ; O=C(c1ccc(F)cc1)N1CCN(Cc2c(-c3ccc(Cl)cc3)nc3cnc(-c4ccccc4)cn23)CC1 ; O=C(c1cccc(F)c1)N1CCCN(Cc2c(-c3ccc(Cl)cc3)nc3ccc(-c4ccccc4)cn23)CC1 ; Cc1c(-c2ccccc2)ccc2nc(-c3ccc(Cl)cc3)c(CN3CCOCC3)n12 ; O=C(C1COC1)N1CCN(Cc2c(-c3ccc(Cl)cc3)nc3ccc(-c4ccccc4)cn23)CC1]

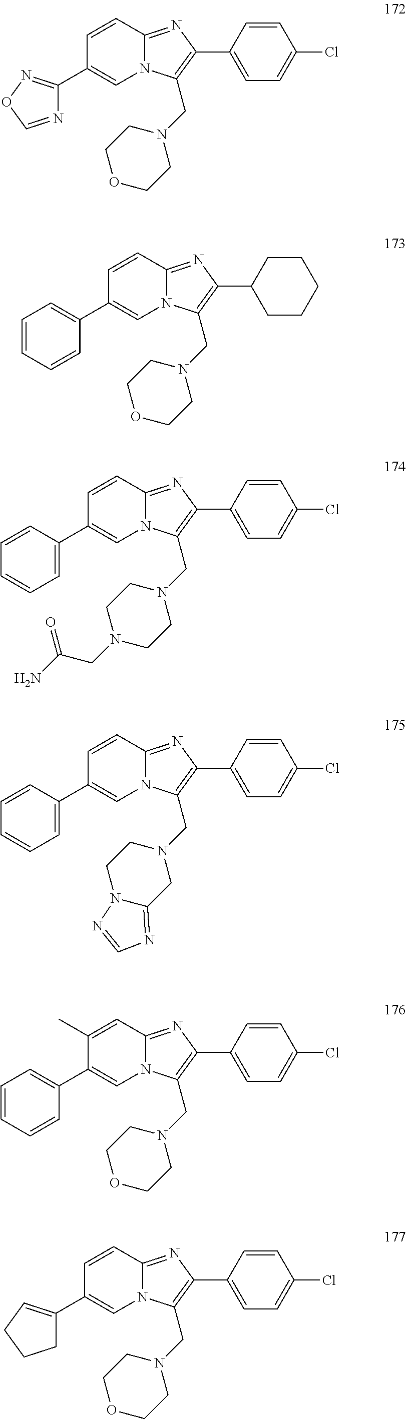 [SMILES: Clc1ccc(-c2nc3ccc(-c4ncon4)cn3c2CN2CCOCC2)cc1 ; c1ccc(-c2ccc3nc(C4CCCCC4)c(CN4CCOCC4)n3c2)cc1 ; NC(=O)CN1CCN(Cc2c(-c3ccc(Cl)cc3)nc3ccc(-c4ccccc4)cn23)CC1 ; Clc1ccc(-c2nc3ccc(-c4ccccc4)cn3c2CN2CCn3ncnc3C2)cc1 ; Cc1cc2nc(-c3ccc(Cl)cc3)c(CN3CCOCC3)n2cc1-c1ccccc1 ; Clc1ccc(-c2nc3ccc(C4=CCCC4)cn3c2CN2CCOCC2)cc1]

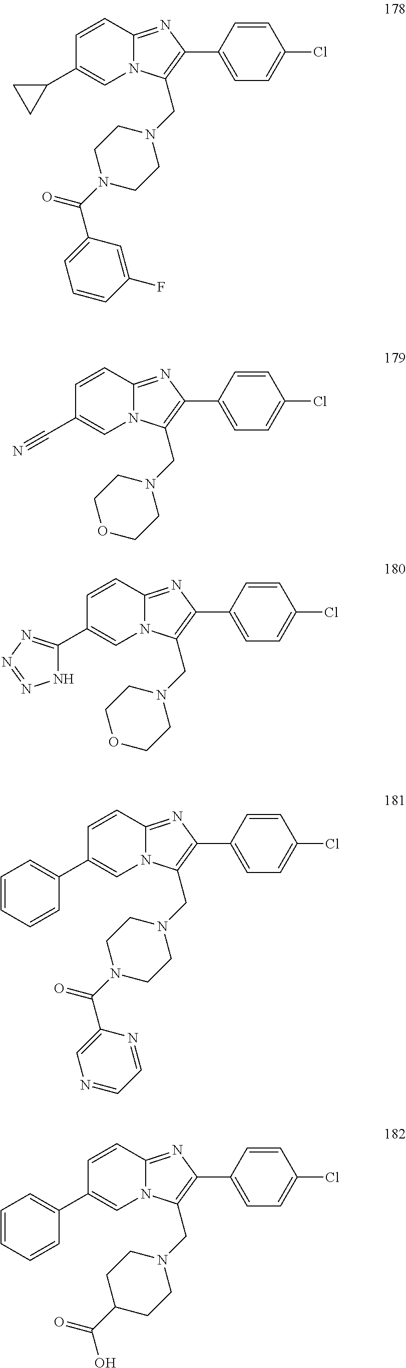

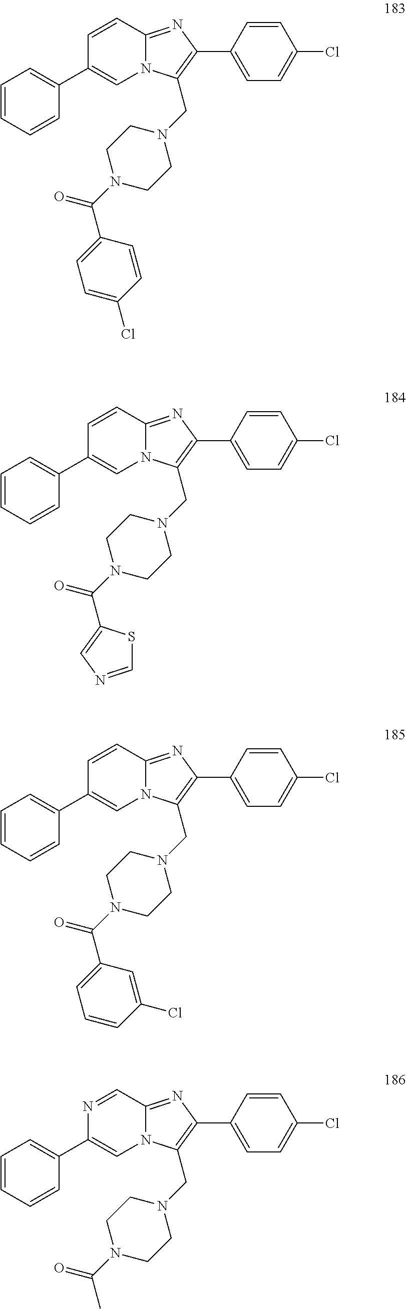 [SMILES: O=C(c1ccc(Cl)cc1)N1CCN(Cc2c(-c3ccc(Cl)cc3)nc3ccc(-c4ccccc4)cn23)CC1 ; O=C(c1cncs1)N1CCN(Cc2c(-c3ccc(Cl)cc3)nc3ccc(-c4ccccc4)cn23)CC1 ; O=C(c1cccc(Cl)c1)N1CCN(Cc2c(-c3ccc(Cl)cc3)nc3ccc(-c4ccccc4)cn23)CC1 ; CC(=O)N1CCN(Cc2c(-c3ccc(Cl)cc3)nc3cnc(-c4ccccc4)cn23)CC1]

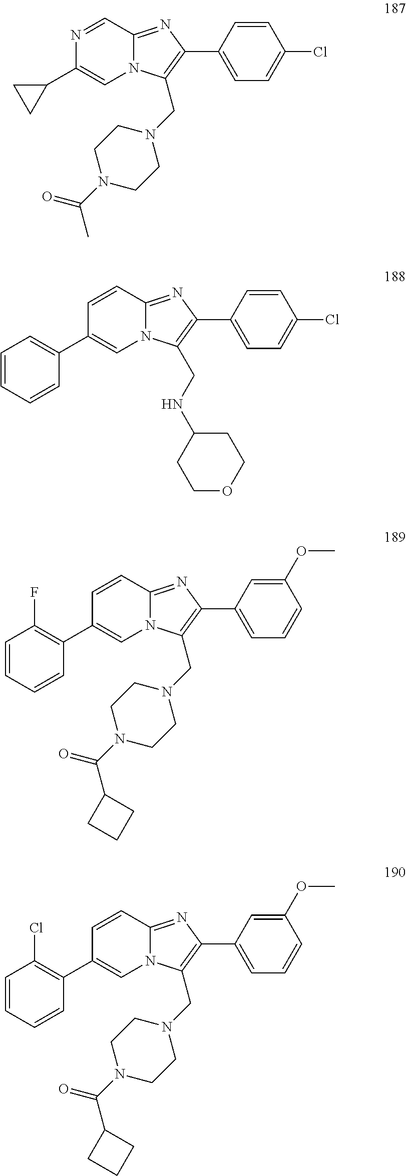 [SMILES: CC(=O)N1CCN(Cc2c(-c3ccc(Cl)cc3)nc3cnc(C4CC4)cn23)CC1 ; Clc1ccc(-c2nc3ccc(-c4ccccc4)cn3c2CNC2CCOCC2)cc1 ; COc1cccc(-c2nc3ccc(-c4ccccc4F)cn3c2CN2CCN(C(=O)C3CCC3)CC2)c1 ; COc1cccc(-c2nc3ccc(-c4ccccc4Cl)cn3c2CN2CCN(C(=O)C3CCC3)CC2)c1]

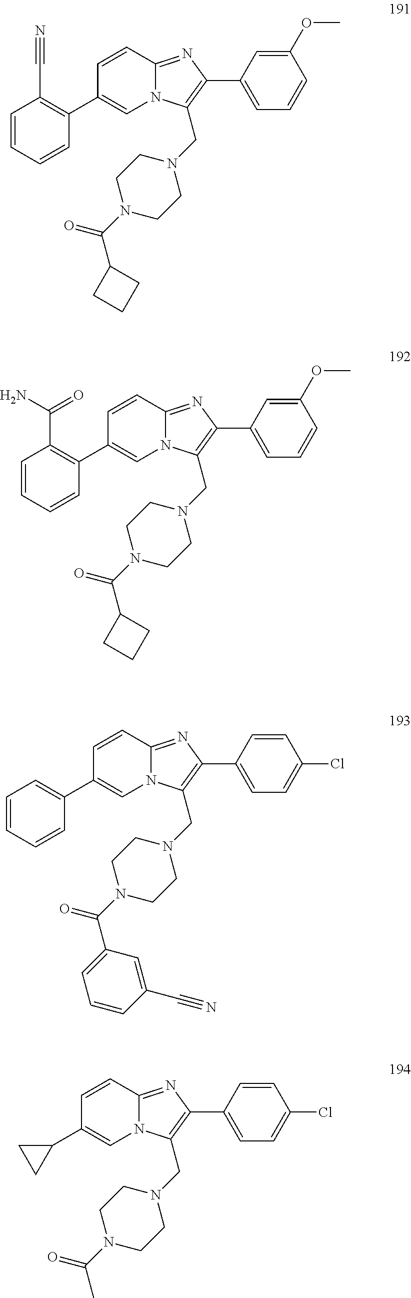 [SMILES: COc1cccc(-c2nc3ccc(-c4ccccc4C#N)cn3c2CN2CCN(C(=O)C3CCC3)CC2)c1 ; COc1cccc(-c2nc3ccc(-c4ccccc4C(N)=O)cn3c2CN2CCN(C(=O)C3CCC3)CC2)c1 ; N#Cc1cccc(C(=O)N2CCN(Cc3c(-c4ccc(Cl)cc4)nc4ccc(-c5ccccc5)cn34)CC2)c1 ; CC(=O)N1CCN(Cc2c(-c3ccc(Cl)cc3)nc3ccc(C4CC4)cn23)CC1]

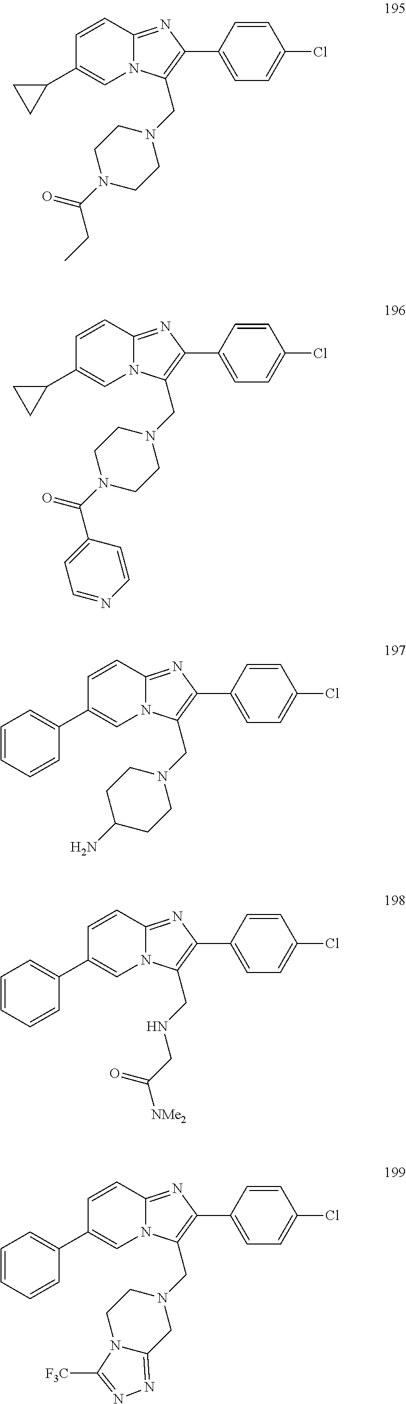 [SMILES: CCC(=O)N1CCN(Cc2c(-c3ccc(Cl)cc3)nc3ccc(C4CC4)cn23)CC1 ; O=C(c1ccncc1)N1CCN(Cc2c(-c3ccc(Cl)cc3)nc3ccc(C4CC4)cn23)CC1 ; NC1CCN(Cc2c(-c3ccc(Cl)cc3)nc3ccc(-c4ccccc4)cn23)CC1 ; CN(C)C(=O)CNCc1c(-c2ccc(Cl)cc2)nc2ccc(-c3ccccc3)cn12 ; FC(F)(F)c1nnc2n1CCN(Cc1c(-c3ccc(Cl)cc3)nc3ccc(-c4ccccc4)cn13)C2]

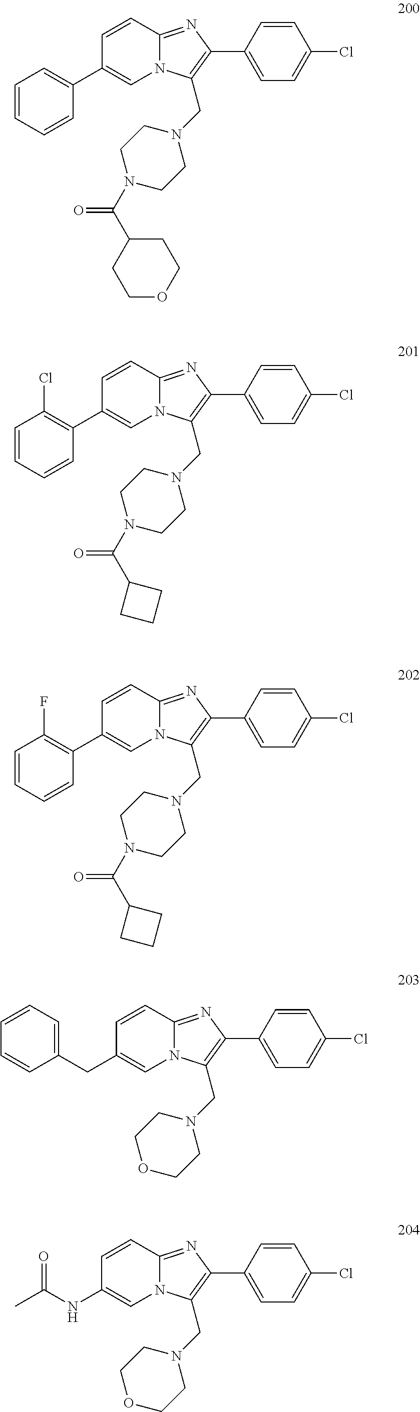 [SMILES: O=C(C1CCOCC1)N1CCN(Cc2c(-c3ccc(Cl)cc3)nc3ccc(-c4ccccc4)cn23)CC1 ; O=C(C1CCC1)N1CCN(Cc2c(-c3ccc(Cl)cc3)nc3ccc(-c4ccccc4Cl)cn23)CC1 ; O=C(C1CCC1)N1CCN(Cc2c(-c3ccc(Cl)cc3)nc3ccc(-c4ccccc4F)cn23)CC1 ; Clc1ccc(-c2nc3ccc(Cc4ccccc4)cn3c2CN2CCOCC2)cc1 ; CC(=O)Nc1ccc2nc(-c3ccc(Cl)cc3)c(CN3CCOCC3)n2c1]

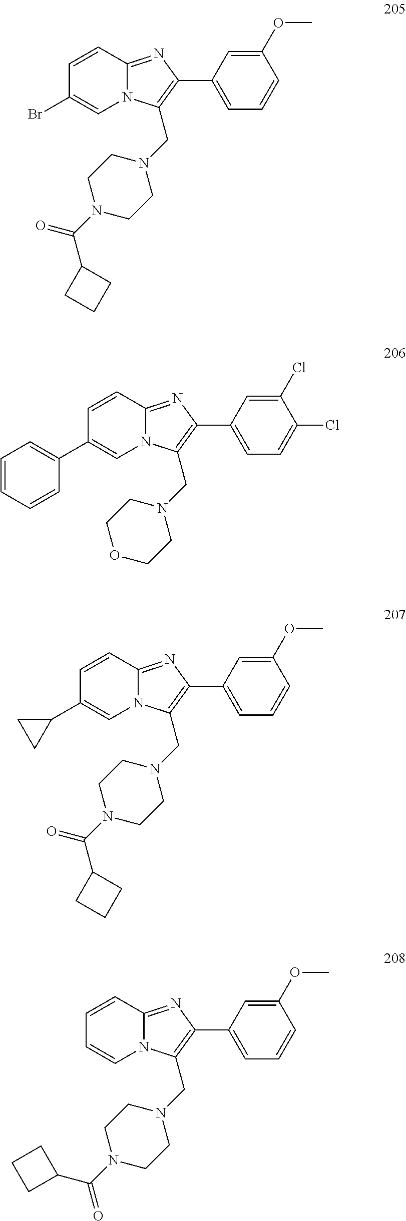 [SMILES: COc1cccc(-c2nc3ccc(Br)cn3c2CN2CCN(C(=O)C3CCC3)CC2)c1 ; Clc1ccc(-c2nc3ccc(-c4ccccc4)cn3c2CN2CCOCC2)cc1Cl ; COc1cccc(-c2nc3ccc(C4CC4)cn3c2CN2CCN(C(=O)C3CCC3)CC2)c1 ; COc1cccc(-c2nc3ccccn3c2CN2CCN(C(=O)C3CCC3)CC2)c1]

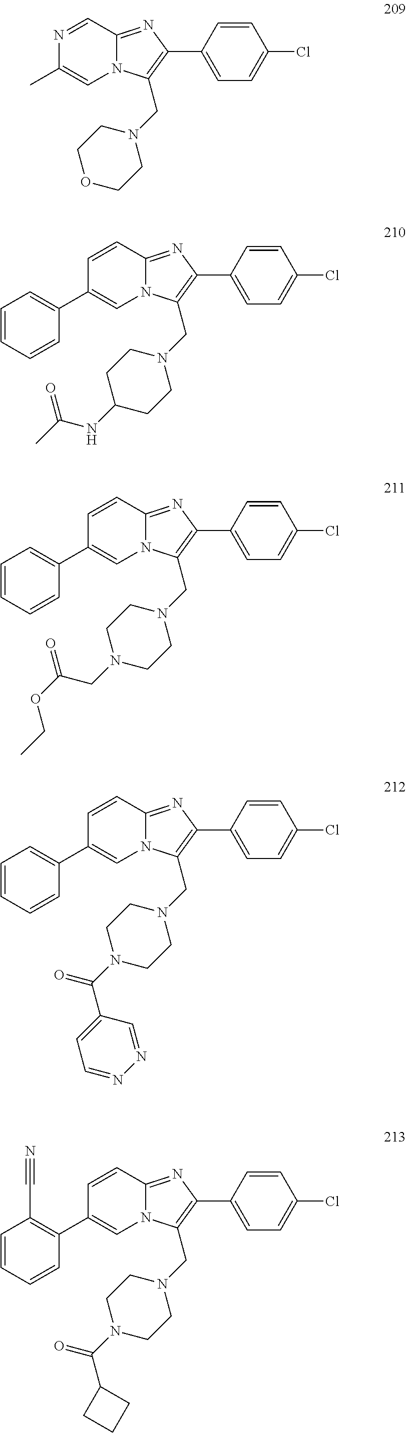 [SMILES: Cc1cn2c(CN3CCOCC3)c(-c3ccc(Cl)cc3)nc2cn1 ; CC(=O)NC1CCN(Cc2c(-c3ccc(Cl)cc3)nc3ccc(-c4ccccc4)cn23)CC1 ; CCOC(=O)CN1CCN(Cc2c(-c3ccc(Cl)cc3)nc3ccc(-c4ccccc4)cn23)CC1 ; O=C(c1ccnnc1)N1CCN(Cc2c(-c3ccc(Cl)cc3)nc3ccc(-c4ccccc4)cn23)CC1 ; N#Cc1ccccc1-c1ccc2nc(-c3ccc(Cl)cc3)c(CN3CCN(C(=O)C4CCC4)CC3)n2c1]

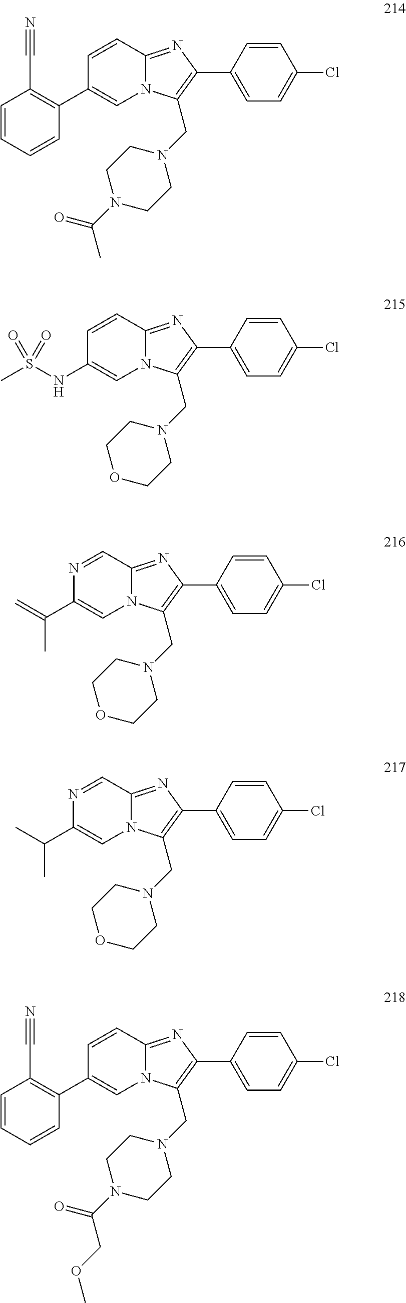 [SMILES: CC(=O)N1CCN(Cc2c(-c3ccc(Cl)cc3)nc3ccc(-c4ccccc4C#N)cn23)CC1 ; CS(=O)(=O)Nc1ccc2nc(-c3ccc(Cl)cc3)c(CN3CCOCC3)n2c1 ; C=C(C)c1cn2c(CN3CCOCC3)c(-c3ccc(Cl)cc3)nc2cn1 ; CC(C)c1cn2c(CN3CCOCC3)c(-c3ccc(Cl)cc3)nc2cn1 ; COCC(=O)N1CCN(Cc2c(-c3ccc(Cl)cc3)nc3ccc(-c4ccccc4C#N)cn23)CC1]

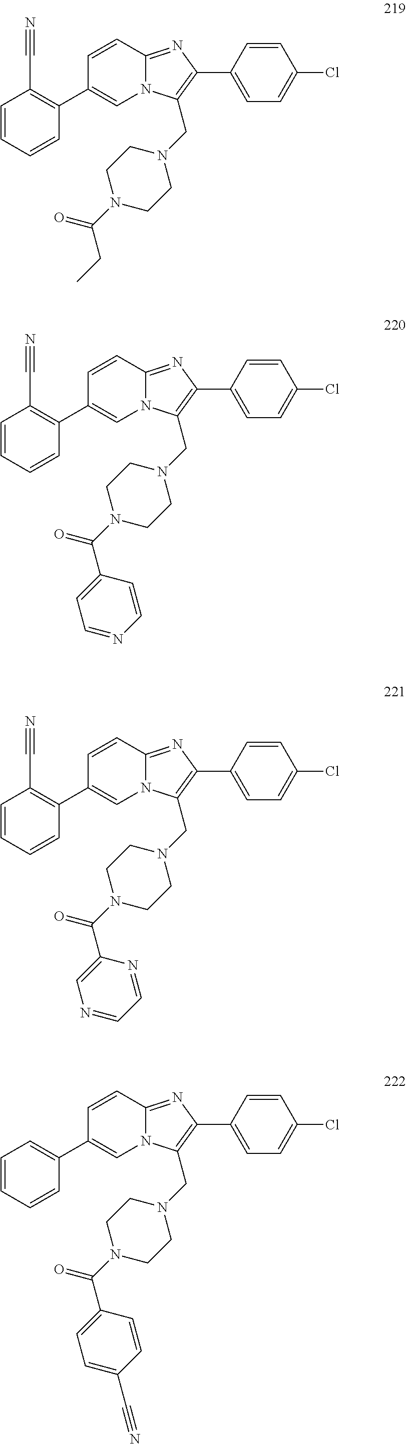 [SMILES: CCC(=O)N1CCN(Cc2c(-c3ccc(Cl)cc3)nc3ccc(-c4ccccc4C#N)cn23)CC1 ; N#Cc1ccccc1-c1ccc2nc(-c3ccc(Cl)cc3)c(CN3CCN(C(=O)c4ccncc4)CC3)n2c1 ; N#Cc1ccccc1-c1ccc2nc(-c3ccc(Cl)cc3)c(CN3CCN(C(=O)c4cnccn4)CC3)n2c1 ; N#Cc1ccc(C(=O)N2CCN(Cc3c(-c4ccc(Cl)cc4)nc4ccc(-c5ccccc5)cn34)CC2)cc1]

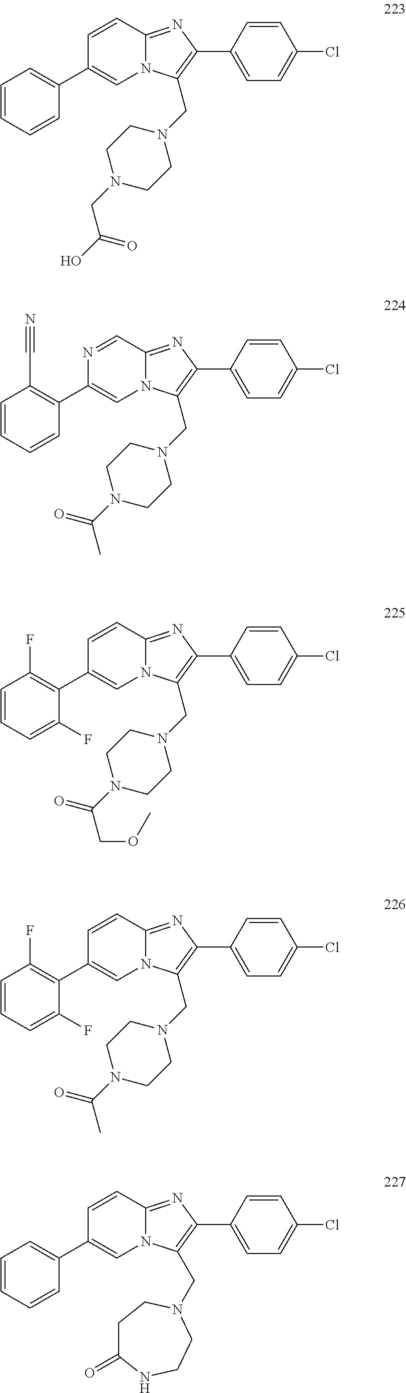 [SMILES: O=C(O)CN1CCN(Cc2c(-c3ccc(Cl)cc3)nc3ccc(-c4ccccc4)cn23)CC1 ; CC(=O)N1CCN(Cc2c(-c3ccc(Cl)cc3)nc3cnc(-c4ccccc4C#N)cn23)CC1 ; COCC(=O)N1CCN(Cc2c(-c3ccc(Cl)cc3)nc3ccc(-c4c(F)cccc4F)cn23)CC1 ; CC(=O)N1CCN(Cc2c(-c3ccc(Cl)cc3)nc3ccc(-c4c(F)cccc4F)cn23)CC1 ; O=C1CCN(Cc2c(-c3ccc(Cl)cc3)nc3ccc(-c4ccccc4)cn23)CCN1]

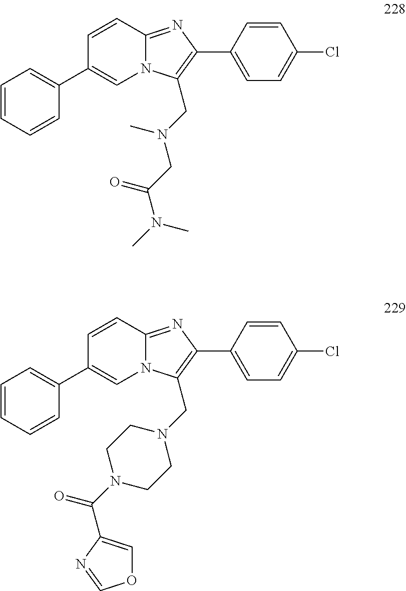 [SMILES: CN(CC(=O)N(C)C)Cc1c(-c2ccc(Cl)cc2)nc2ccc(-c3ccccc3)cn12 ; O=C(c1cocn1)N1CCN(Cc2c(-c3ccc(Cl)cc3)nc3ccc(-c4ccccc4)cn23)CC1]

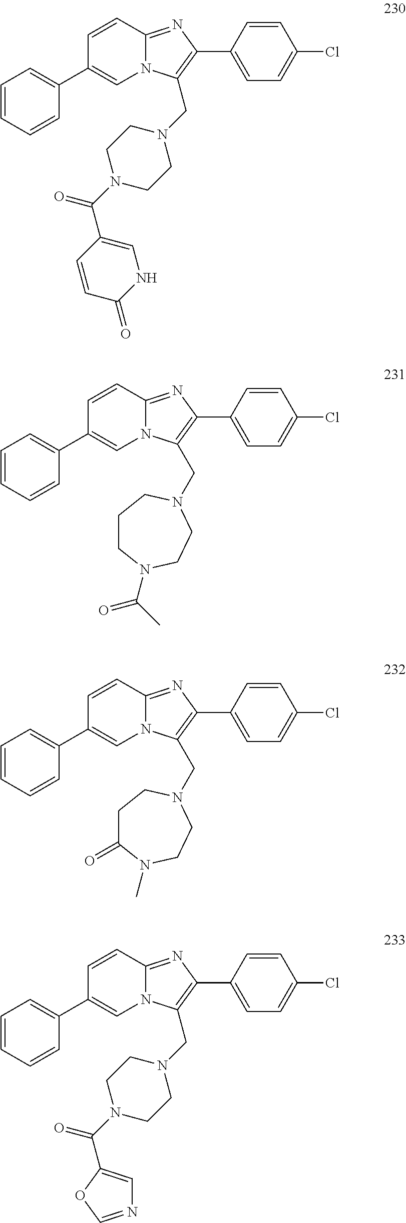 [SMILES: O=C(c1ccc(=O)[nH]c1)N1CCN(Cc2c(-c3ccc(Cl)cc3)nc3ccc(-c4ccccc4)cn23)CC1 ; CC(=O)N1CCCN(Cc2c(-c3ccc(Cl)cc3)nc3ccc(-c4ccccc4)cn23)CC1 ; CN1CCN(Cc2c(-c3ccc(Cl)cc3)nc3ccc(-c4ccccc4)cn23)CCC1=O ; O=C(c1cnco1)N1CCN(Cc2c(-c3ccc(Cl)cc3)nc3ccc(-c4ccccc4)cn23)CC1]

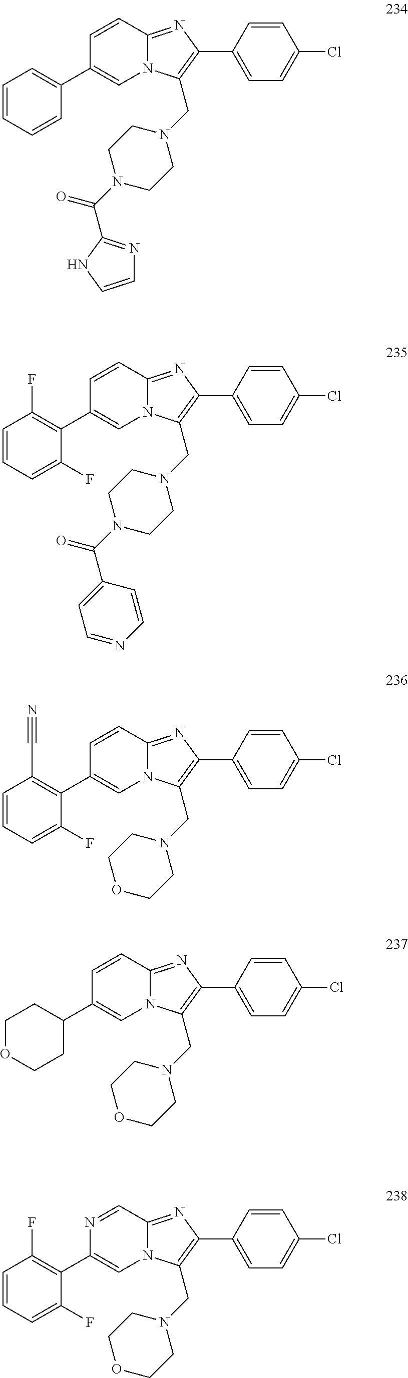 [SMILES: O=C(c1ncc[nH]1)N1CCN(Cc2c(-c3ccc(Cl)cc3)nc3ccc(-c4ccccc4)cn23)CC1 ; O=C(c1ccncc1)N1CCN(Cc2c(-c3ccc(Cl)cc3)nc3ccc(-c4c(F)cccc4F)cn23)CC1 ; N#Cc1cccc(F)c1-c1ccc2nc(-c3ccc(Cl)cc3)c(CN3CCOCC3)n2c1 ; Clc1ccc(-c2nc3ccc(C4CCOCC4)cn3c2CN2CCOCC2)cc1 ; Fc1cccc(F)c1-c1cn2c(CN3CCOCC3)c(-c3ccc(Cl)cc3)nc2cn1]

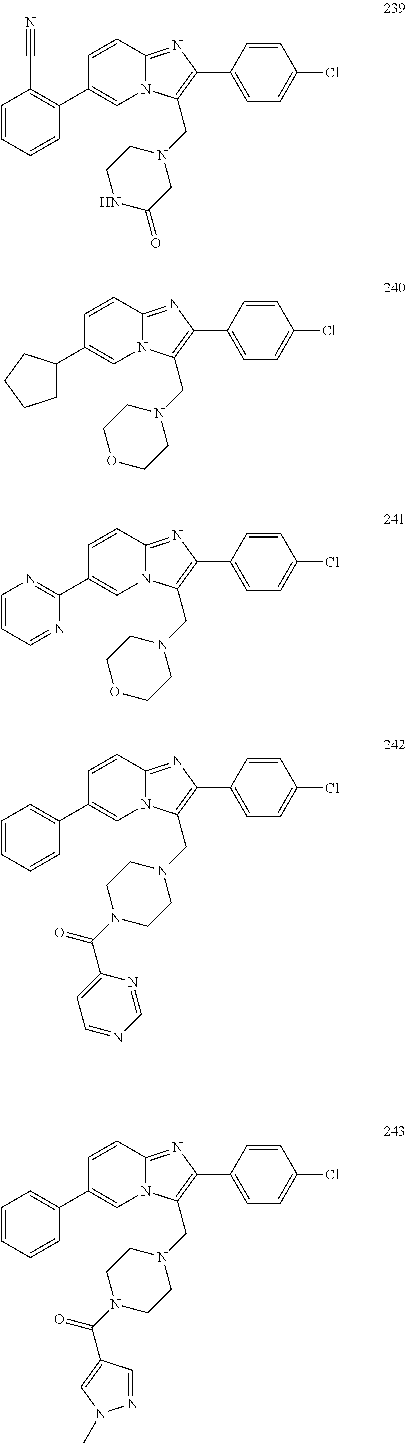 [SMILES: N#Cc1ccccc1-c1ccc2nc(-c3ccc(Cl)cc3)c(CN3CCNC(=O)C3)n2c1 ; Clc1ccc(-c2nc3ccc(C4CCCC4)cn3c2CN2CCOCC2)cc1 ; Clc1ccc(-c2nc3ccc(-c4ncccn4)cn3c2CN2CCOCC2)cc1 ; O=C(c1ccncn1)N1CCN(Cc2c(-c3ccc(Cl)cc3)nc3ccc(-c4ccccc4)cn23)CC1 ; Cn1cc(C(=O)N2CCN(Cc3c(-c4ccc(Cl)cc4)nc4ccc(-c5ccccc5)cn34)CC2)cn1]

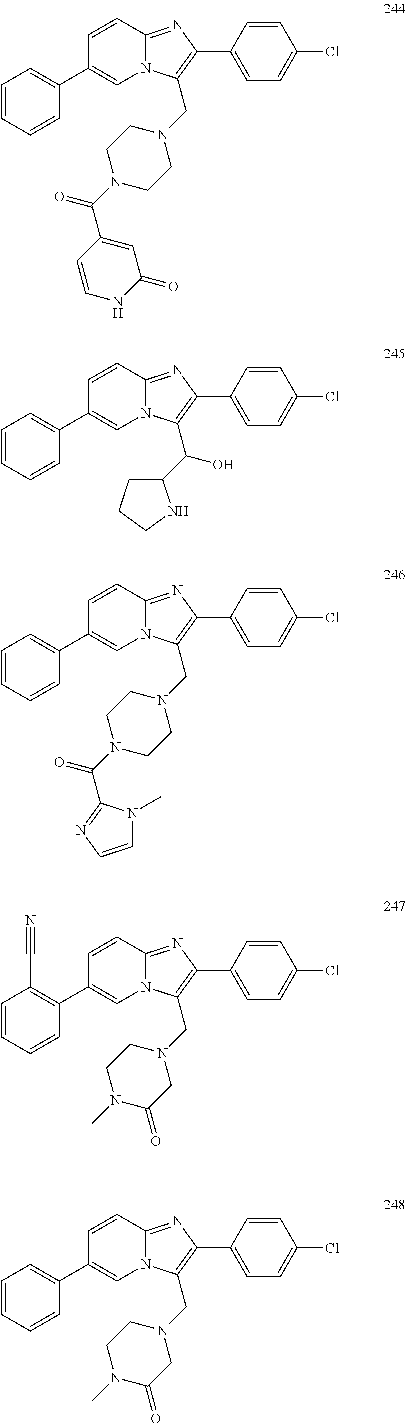 [SMILES: O=C(c1cc[nH]c(=O)c1)N1CCN(Cc2c(-c3ccc(Cl)cc3)nc3ccc(-c4ccccc4)cn23)CC1 ; OC(c1c(-c2ccc(Cl)cc2)nc2ccc(-c3ccccc3)cn12)C1CCCN1 ; Cn1ccnc1C(=O)N1CCN(Cc2c(-c3ccc(Cl)cc3)nc3ccc(-c4ccccc4)cn23)CC1 ; CN1CCN(Cc2c(-c3ccc(Cl)cc3)nc3ccc(-c4ccccc4C#N)cn23)CC1=O ; CN1CCN(Cc2c(-c3ccc(Cl)cc3)nc3ccc(-c4ccccc4)cn23)CC1=O]

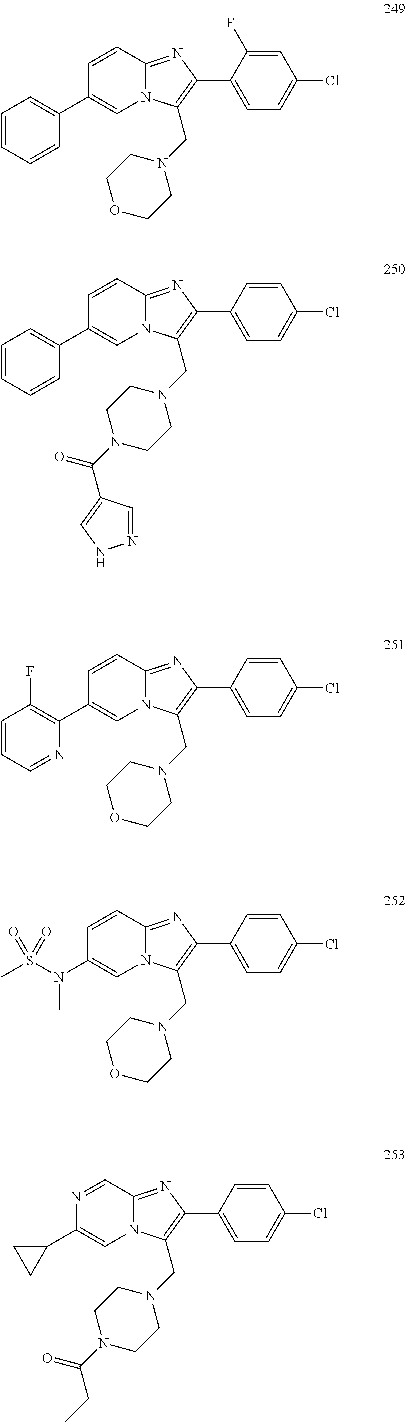 [SMILES: Fc1cc(Cl)ccc1-c1nc2ccc(-c3ccccc3)cn2c1CN1CCOCC1 ; O=C(c1cn[nH]c1)N1CCN(Cc2c(-c3ccc(Cl)cc3)nc3ccc(-c4ccccc4)cn23)CC1 ; Fc1cccnc1-c1ccc2nc(-c3ccc(Cl)cc3)c(CN3CCOCC3)n2c1 ; CN(c1ccc2nc(-c3ccc(Cl)cc3)c(CN3CCOCC3)n2c1)S(C)(=O)=O ; CCC(=O)N1CCN(Cc2c(-c3ccc(Cl)cc3)nc3cnc(C4CC4)cn23)CC1]

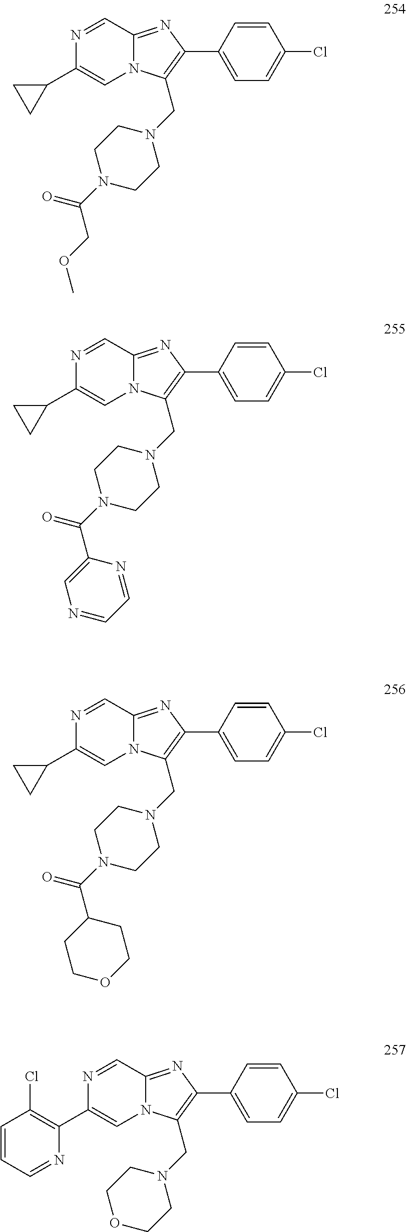 [SMILES: COCC(=O)N1CCN(Cc2c(-c3ccc(Cl)cc3)nc3cnc(C4CC4)cn23)CC1 ; O=C(c1cnccn1)N1CCN(Cc2c(-c3ccc(Cl)cc3)nc3cnc(C4CC4)cn23)CC1 ; O=C(C1CCOCC1)N1CCN(Cc2c(-c3ccc(Cl)cc3)nc3cnc(C4CC4)cn23)CC1 ; Clc1ccc(-c2nc3cnc(-c4ncccc4Cl)cn3c2CN2CCOCC2)cc1]

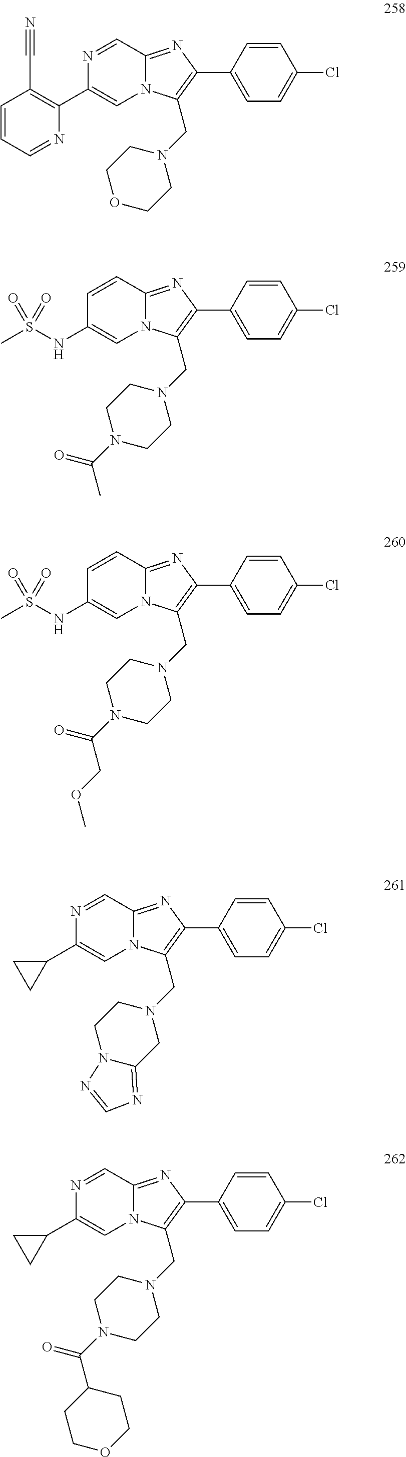 [SMILES: N#Cc1cccnc1-c1cn2c(CN3CCOCC3)c(-c3ccc(Cl)cc3)nc2cn1 ; CC(=O)N1CCN(Cc2c(-c3ccc(Cl)cc3)nc3ccc(NS(C)(=O)=O)cn23)CC1 ; COCC(=O)N1CCN(Cc2c(-c3ccc(Cl)cc3)nc3ccc(NS(C)(=O)=O)cn23)CC1 ; Clc1ccc(-c2nc3cnc(C4CC4)cn3c2CN2CCn3ncnc3C2)cc1 ; O=C(C1CCOCC1)N1CCN(Cc2c(-c3ccc(Cl)cc3)nc3cnc(C4CC4)cn23)CC1]

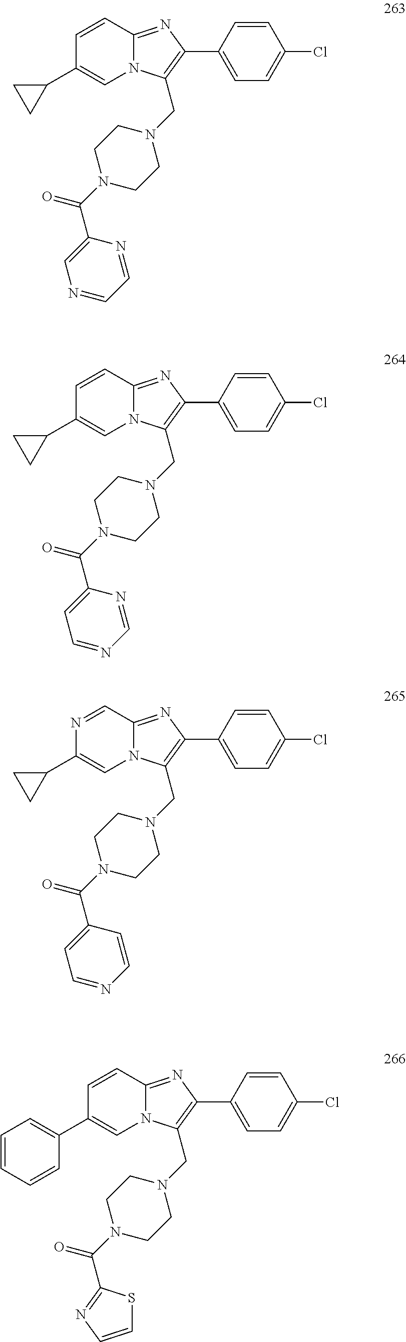 [SMILES: O=C(c1cnccn1)N1CCN(Cc2c(-c3ccc(Cl)cc3)nc3ccc(C4CC4)cn23)CC1 ; O=C(c1ccncn1)N1CCN(Cc2c(-c3ccc(Cl)cc3)nc3ccc(C4CC4)cn23)CC1 ; O=C(c1ccncc1)N1CCN(Cc2c(-c3ccc(Cl)cc3)nc3cnc(C4CC4)cn23)CC1 ; O=C(c1nccs1)N1CCN(Cc2c(-c3ccc(Cl)cc3)nc3ccc(-c4ccccc4)cn23)CC1]

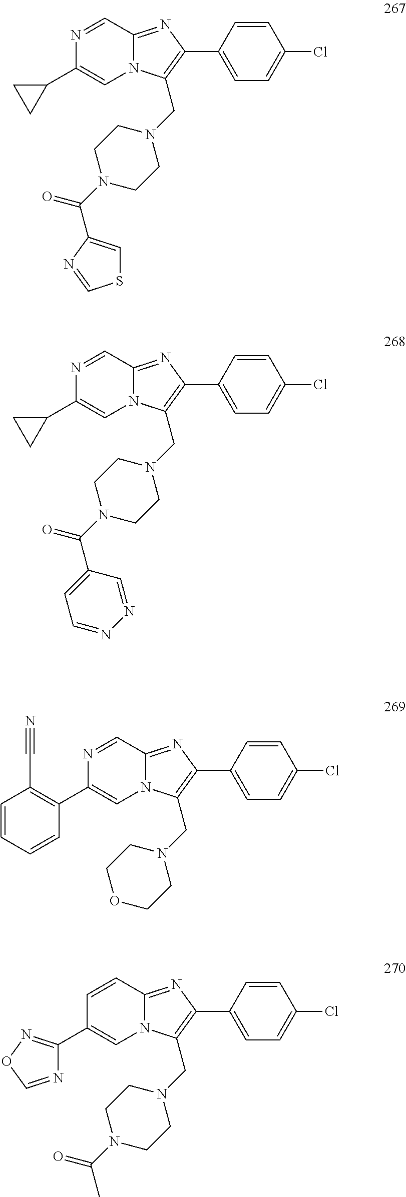 [SMILES: O=C(c1cscn1)N1CCN(Cc2c(-c3ccc(Cl)cc3)nc3cnc(C4CC4)cn23)CC1 ; O=C(c1ccnnc1)N1CCN(Cc2c(-c3ccc(Cl)cc3)nc3cnc(C4CC4)cn23)CC1 ; N#Cc1ccccc1-c1cn2c(CN3CCOCC3)c(-c3ccc(Cl)cc3)nc2cn1 ; CC(=O)N1CCN(Cc2c(-c3ccc(Cl)cc3)nc3ccc(-c4ncon4)cn23)CC1]

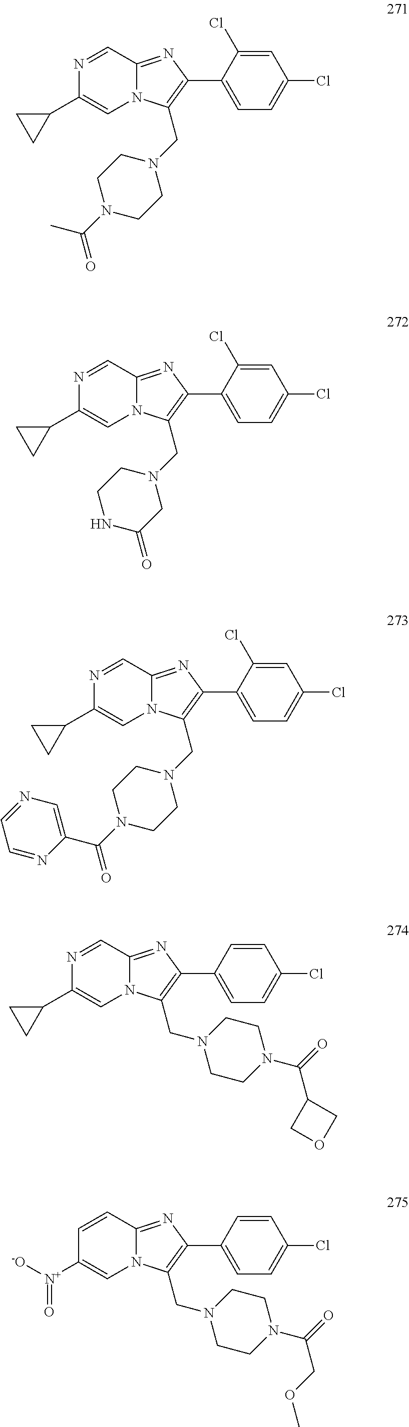

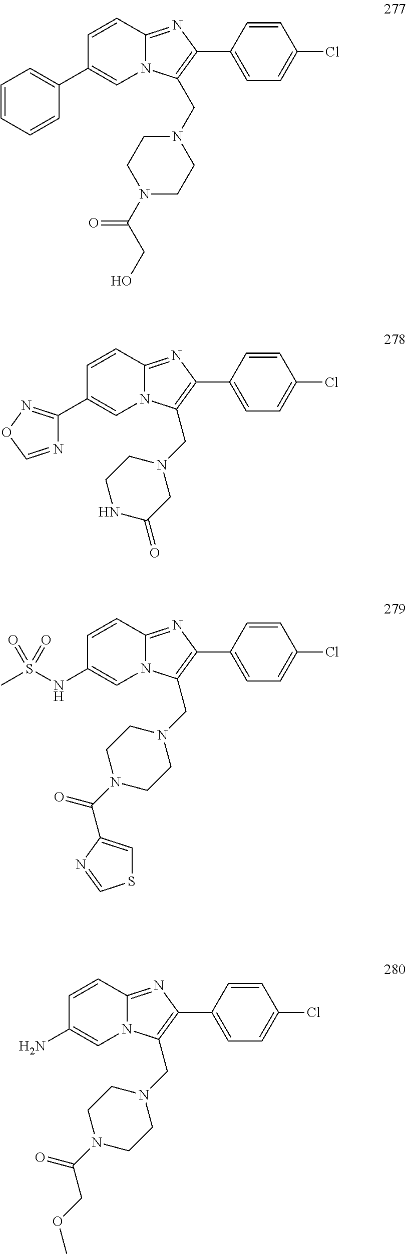 [SMILES: O=C(CO)N1CCN(Cc2c(-c3ccc(Cl)cc3)nc3ccc(-c4ccccc4)cn23)CC1 ; O=C1CN(Cc2c(-c3ccc(Cl)cc3)nc3ccc(-c4ncon4)cn23)CCN1 ; CS(=O)(=O)Nc1ccc2nc(-c3ccc(Cl)cc3)c(CN3CCN(C(=O)c4cscn4)CC3)n2c1 ; COCC(=O)N1CCN(Cc2c(-c3ccc(Cl)cc3)nc3ccc(N)cn23)CC1]

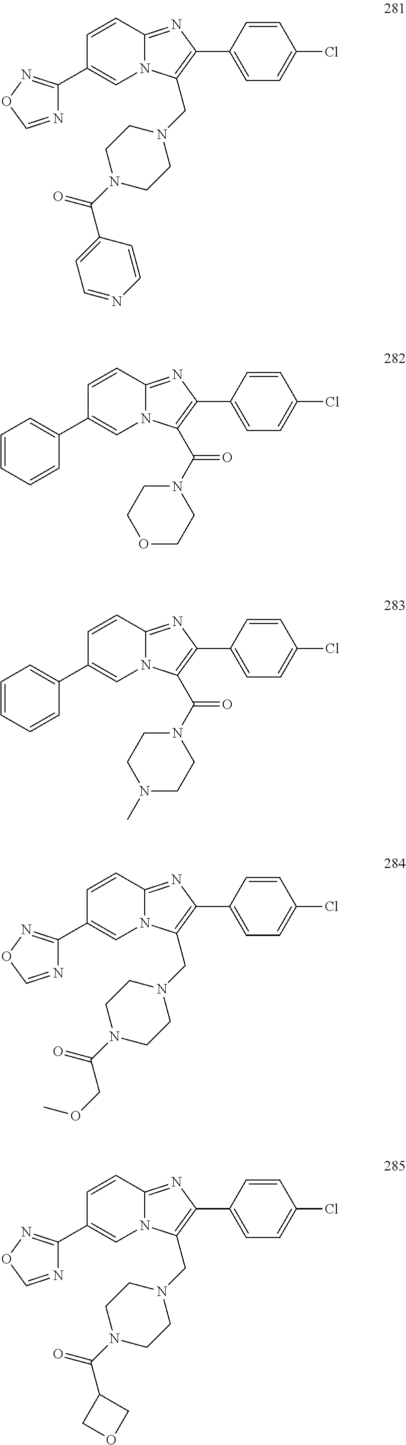 [SMILES: O=C(c1ccncc1)N1CCN(Cc2c(-c3ccc(Cl)cc3)nc3ccc(-c4ncon4)cn23)CC1 ; O=C(c1c(-c2ccc(Cl)cc2)nc2ccc(-c3ccccc3)cn12)N1CCOCC1 ; CN1CCN(C(=O)c2c(-c3ccc(Cl)cc3)nc3ccc(-c4ccccc4)cn23)CC1 ; COCC(=O)N1CCN(Cc2c(-c3ccc(Cl)cc3)nc3ccc(-c4ncon4)cn23)CC1 ; O=C(C1COC1)N1CCN(Cc2c(-c3ccc(Cl)cc3)nc3ccc(-c4ncon4)cn23)CC1]

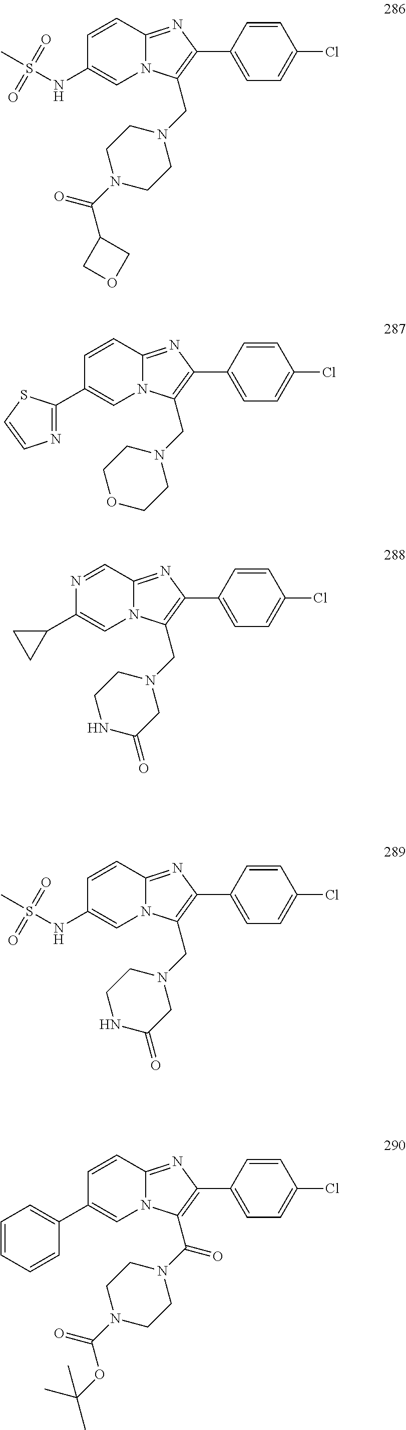 [SMILES: CS(=O)(=O)Nc1ccc2nc(-c3ccc(Cl)cc3)c(CN3CCN(C(=O)C4COC4)CC3)n2c1 ; Clc1ccc(-c2nc3ccc(-c4nccs4)cn3c2CN2CCOCC2)cc1 ; O=C1CN(Cc2c(-c3ccc(Cl)cc3)nc3cnc(C4CC4)cn23)CCN1 ; CS(=O)(=O)Nc1ccc2nc(-c3ccc(Cl)cc3)c(CN3CCNC(=O)C3)n2c1 ; CC(C)(C)OC(=O)N1CCN(C(=O)c2c(-c3ccc(Cl)cc3)nc3ccc(-c4ccccc4)cn23)CC1]

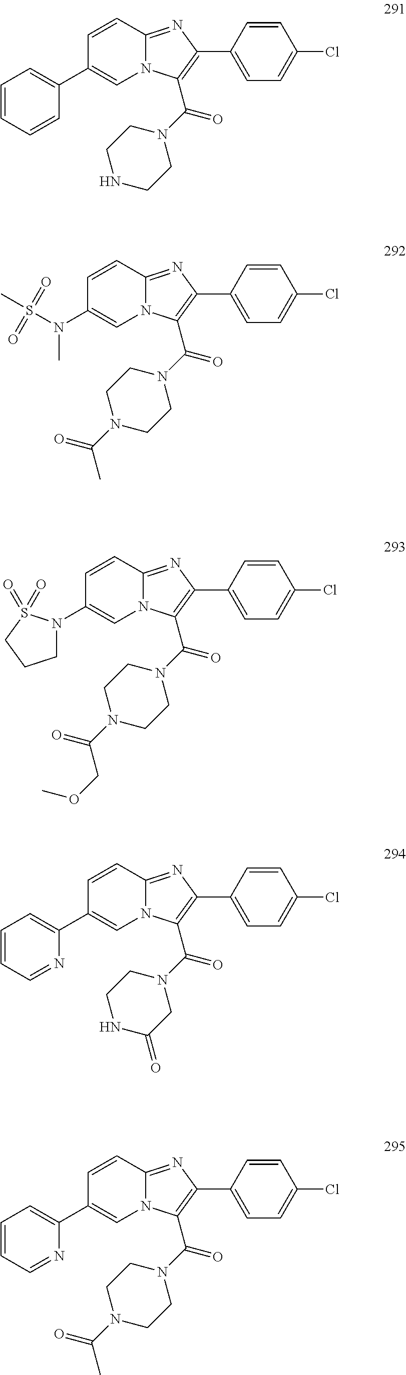 [SMILES: O=C(c1c(-c2ccc(Cl)cc2)nc2ccc(-c3ccccc3)cn12)N1CCNCC1 ; CC(=O)N1CCN(C(=O)c2c(-c3ccc(Cl)cc3)nc3ccc(N(C)S(C)(=O)=O)cn23)CC1 ; COCC(=O)N1CCN(C(=O)c2c(-c3ccc(Cl)cc3)nc3ccc(N4CCCS4(=O)=O)cn23)CC1 ; O=C1CN(C(=O)c2c(-c3ccc(Cl)cc3)nc3ccc(-c4ccccn4)cn23)CCN1 ; CC(=O)N1CCN(C(=O)c2c(-c3ccc(Cl)cc3)nc3ccc(-c4ccccn4)cn23)CC1]

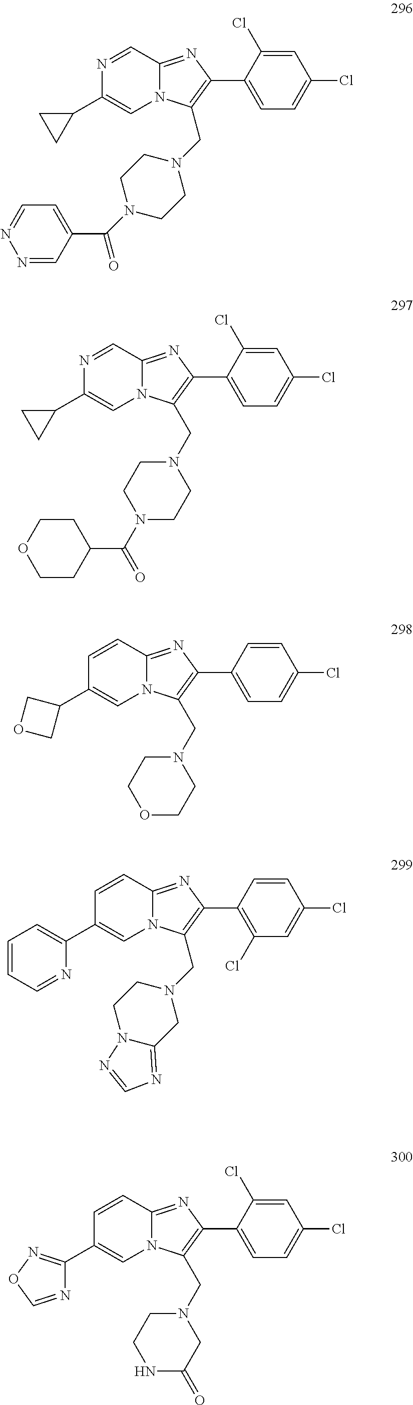 [SMILES: O=C(c1ccnnc1)N1CCN(Cc2c(-c3ccc(Cl)cc3Cl)nc3cnc(C4CC4)cn23)CC1 ; O=C(C1CCOCC1)N1CCN(Cc2c(-c3ccc(Cl)cc3Cl)nc3cnc(C4CC4)cn23)CC1 ; Clc1ccc(-c2nc3ccc(C4COC4)cn3c2CN2CCOCC2)cc1 ; Clc1ccc(-c2nc3ccc(-c4ccccn4)cn3c2CN2CCn3ncnc3C2)c(Cl)c1 ; O=C1CN(Cc2c(-c3ccc(Cl)cc3Cl)nc3ccc(-c4ncon4)cn23)CCN1]

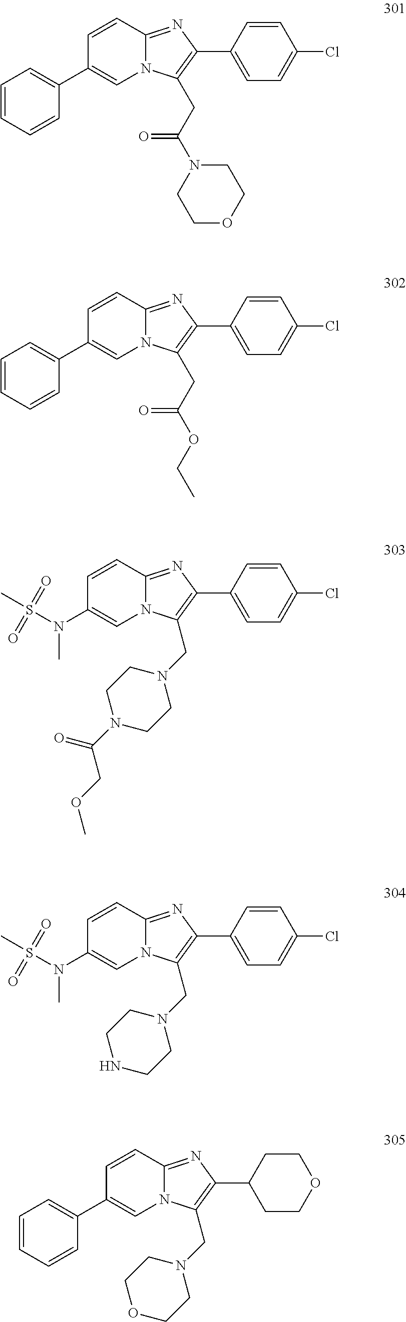 [SMILES: O=C(Cc1c(-c2ccc(Cl)cc2)nc2ccc(-c3ccccc3)cn12)N1CCOCC1 ; CCOC(=O)Cc1c(-c2ccc(Cl)cc2)nc2ccc(-c3ccccc3)cn12 ; COCC(=O)N1CCN(Cc2c(-c3ccc(Cl)cc3)nc3ccc(N(C)S(C)(=O)=O)cn23)CC1 ; CN(c1ccc2nc(-c3ccc(Cl)cc3)c(CN3CCNCC3)n2c1)S(C)(=O)=O ; c1ccc(-c2ccc3nc(C4CCOCC4)c(CN4CCOCC4)n3c2)cc1]

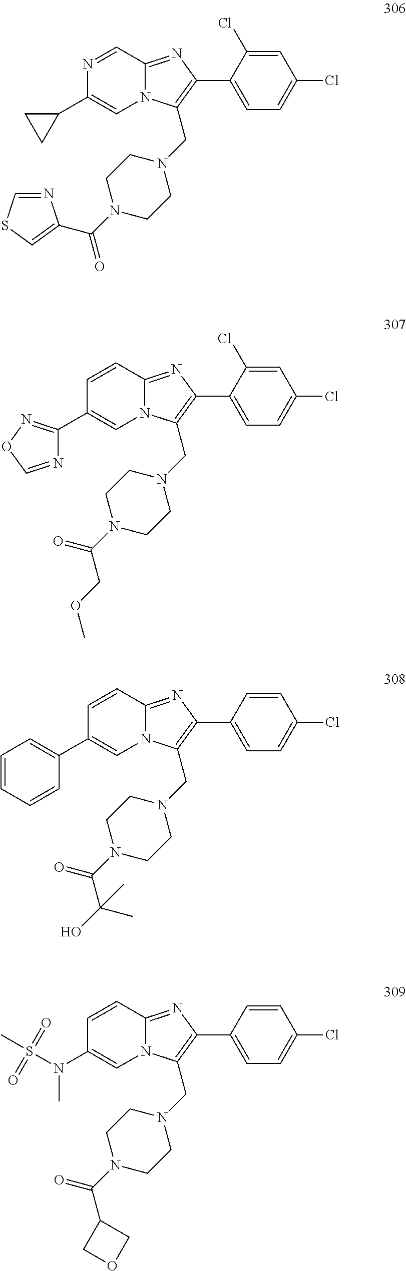 [SMILES: O=C(c1cscn1)N1CCN(Cc2c(-c3ccc(Cl)cc3Cl)nc3cnc(C4CC4)cn23)CC1 ; COCC(=O)N1CCN(Cc2c(-c3ccc(Cl)cc3Cl)nc3ccc(-c4ncon4)cn23)CC1 ; CC(C)(O)C(=O)N1CCN(Cc2c(-c3ccc(Cl)cc3)nc3ccc(-c4ccccc4)cn23)CC1 ; CN(c1ccc2nc(-c3ccc(Cl)cc3)c(CN3CCN(C(=O)C4COC4)CC3)n2c1)S(C)(=O)=O]

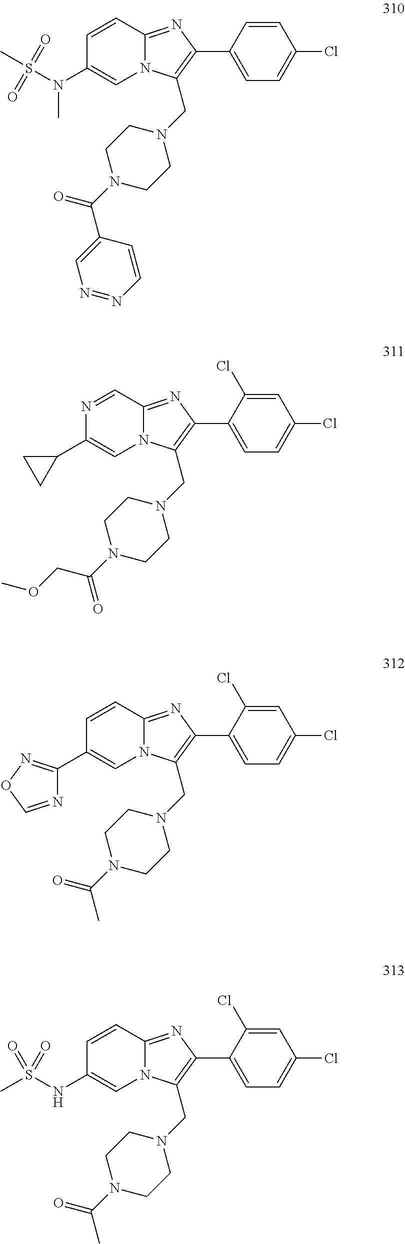 [SMILES: CN(c1ccc2nc(-c3ccc(Cl)cc3)c(CN3CCN(C(=O)c4ccnnc4)CC3)n2c1)S(C)(=O)=O ; COCC(=O)N1CCN(Cc2c(-c3ccc(Cl)cc3Cl)nc3cnc(C4CC4)cn23)CC1 ; CC(=O)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(NS(C)(=O)=O)cn23)CC1]

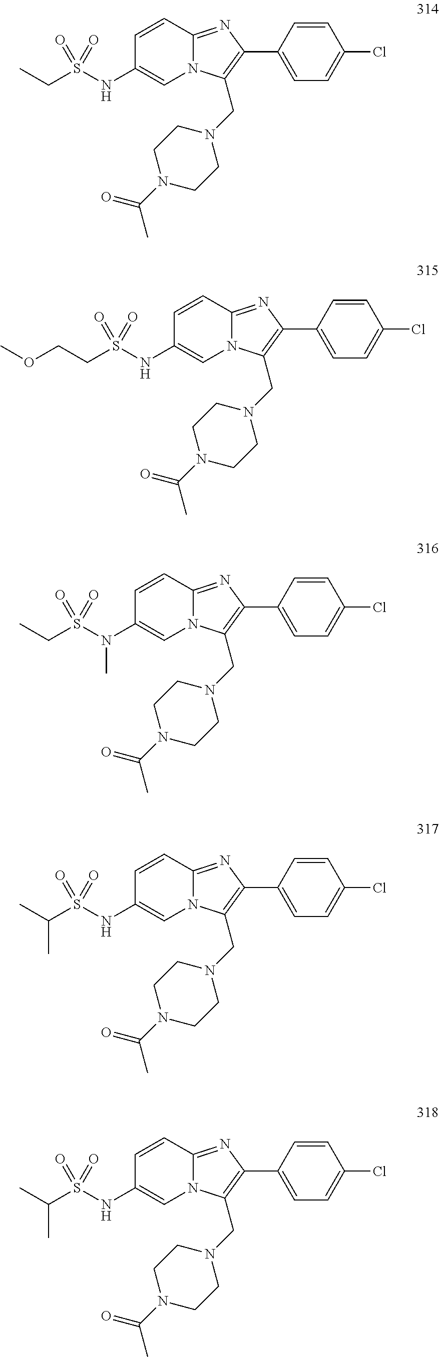 [SMILES: CCS(=O)(=O)Nc1ccc2nc(-c3ccc(Cl)cc3)c(CN3CCN(C(C)=O)CC3)n2c1 ; COCCS(=O)(=O)Nc1ccc2nc(-c3ccc(Cl)cc3)c(CN3CCN(C(C)=O)CC3)n2c1 ; CCS(=O)(=O)N(C)c1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(NS(=O)(=O)C(C)C)cn23)CC1 ; CC(=O)N1CCN(Cc2c(-c3ccc(Cl)cc3)nc3ccc(NS(=O)(=O)C(C)C)cn23)CC1]

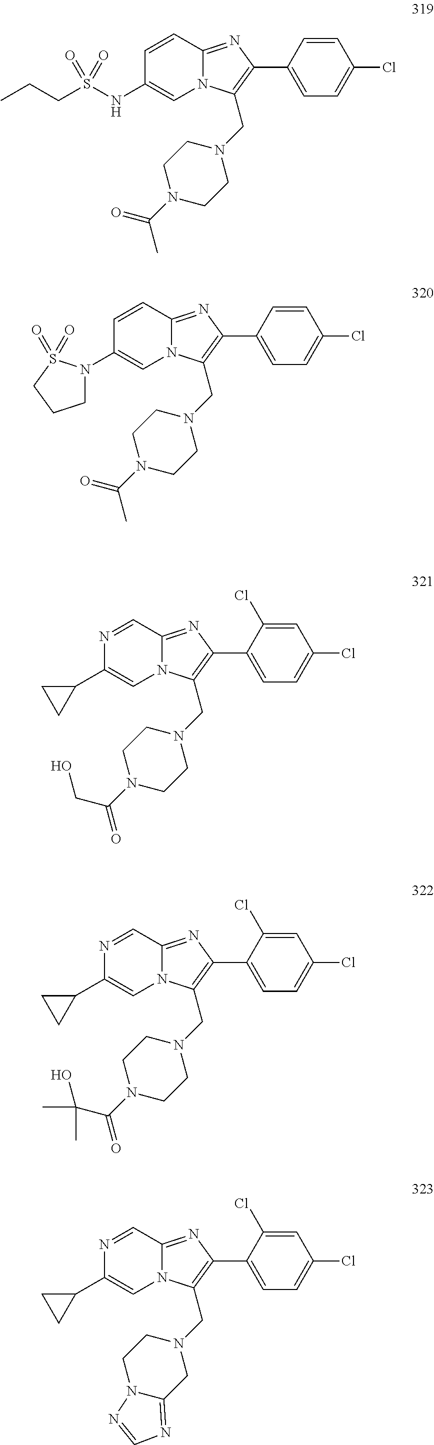 [SMILES: CCCS(=O)(=O)Nc1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(N4CCCS4(=O)=O)cn23)CC1 ; O=C(CO)N1CCN(Cc2c(-c3ccc(Cl)cc3Cl)nc3cnc(C4CC4)cn23)CC1 ; CC(C)(O)C(=O)N1CCN(Cc2c(-c3ccc(Cl)cc3Cl)nc3cnc(C4CC4)cn23)CC1 ; Clc1ccc(-c2nc3cnc(C4CC4)cn3c2CN2CCn3ncnc3C2)c(Cl)c1]

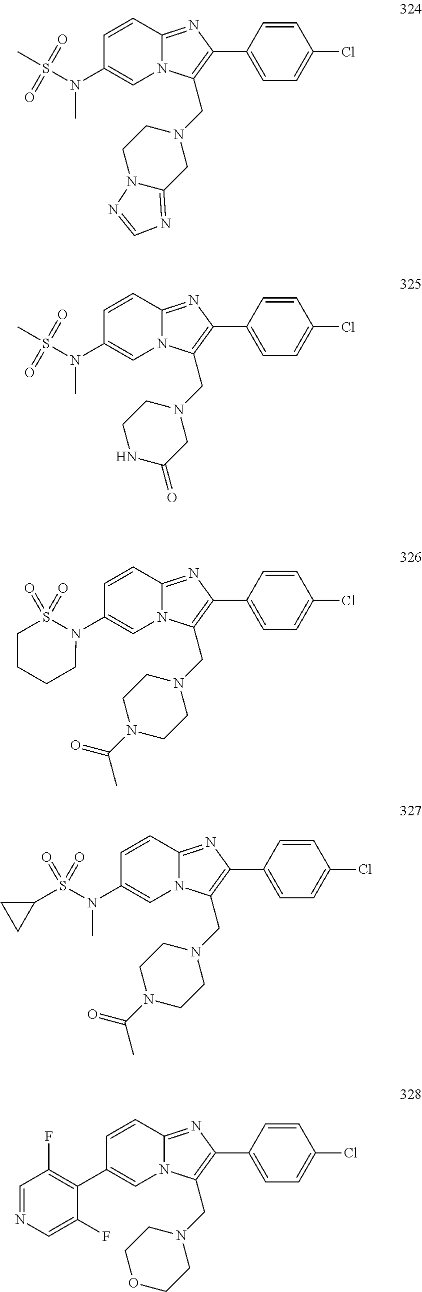 [SMILES: CN(c1ccc2nc(-c3ccc(Cl)cc3)c(CN3CCn4ncnc4C3)n2c1)S(C)(=O)=O ; CN(c1ccc2nc(-c3ccc(Cl)cc3)c(CN3CCNC(=O)C3)n2c1)S(C)(=O)=O ; CC(=O)N1CCN(Cc2c(-c3ccc(Cl)cc3)nc3ccc(N4CCCCS4(=O)=O)cn23)CC1 ; CC(=O)N1CCN(Cc2c(-c3ccc(Cl)cc3)nc3ccc(N(C)S(=O)(=O)C4CC4)cn23)CC1 ; Fc1cncc(F)c1-c1ccc2nc(-c3ccc(Cl)cc3)c(CN3CCOCC3)n2c1]

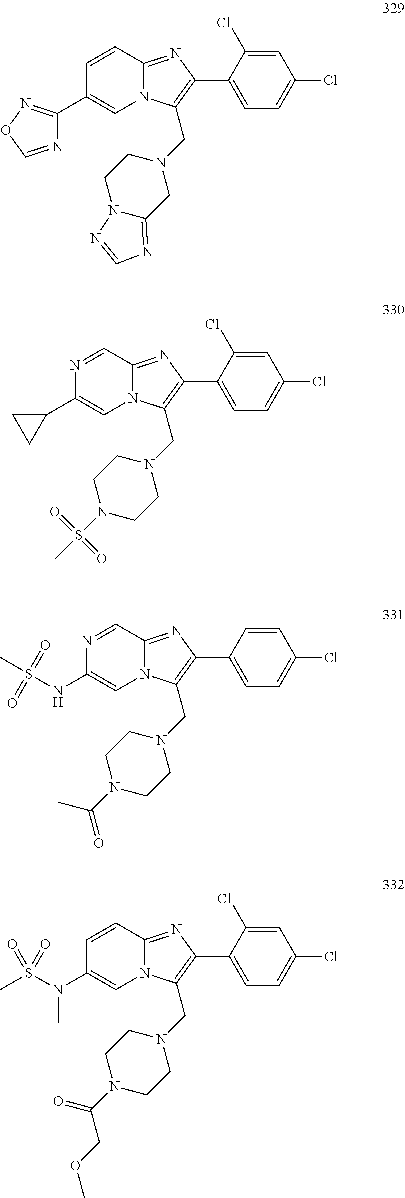 [SMILES: Clc1ccc(-c2nc3ccc(-c4ncon4)cn3c2CN2CCn3ncnc3C2)c(Cl)c1 ; CS(=O)(=O)N1CCN(Cc2c(-c3ccc(Cl)cc3Cl)nc3cnc(C4CC4)cn23)CC1 ; CC(=O)N1CCN(Cc2c(-c3ccc(Cl)cc3)nc3cnc(NS(C)(=O)=O)cn23)CC1 ; COCC(=O)N1CCN(Cc2c(-c3ccc(Cl)cc3Cl)nc3ccc(N(C)S(C)(=O)=O)cn23)CC1]

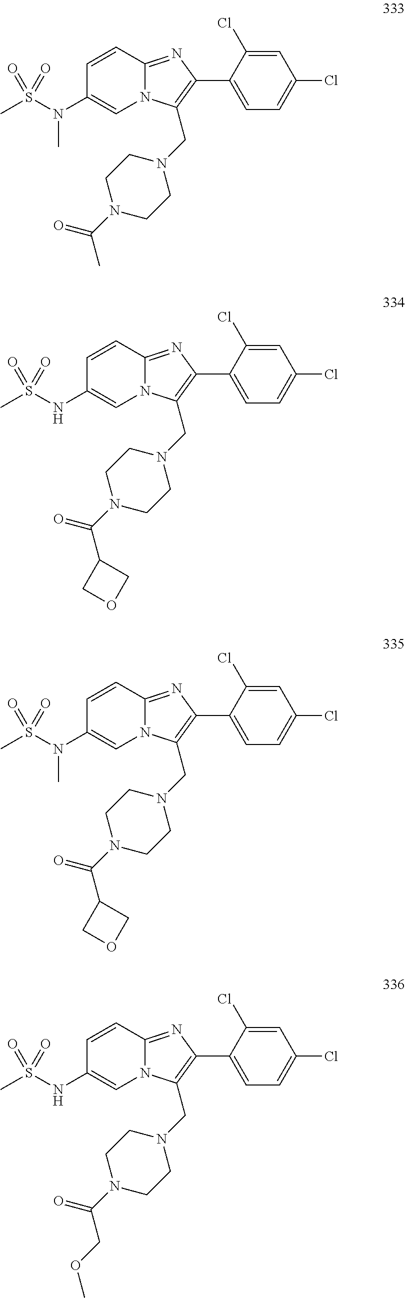 [SMILES: CC(=O)N1CCN(Cc2c(-c3ccc(Cl)cc3Cl)nc3ccc(N(C)S(C)(=O)=O)cn23)CC1 ; CS(=O)(=O)Nc1ccc2nc(-c3ccc(Cl)cc3Cl)c(CN3CCN(C(=O)C4COC4)CC3)n2c1 ; CN(c1ccc2nc(-c3ccc(Cl)cc3Cl)c(CN3CCN(C(=O)C4COC4)CC3)n2c1)S(C)(=O)=O ; COCC(=O)N1CCN(Cc2c(-c3ccc(Cl)cc3Cl)nc3ccc(NS(C)(=O)=O)cn23)CC1]

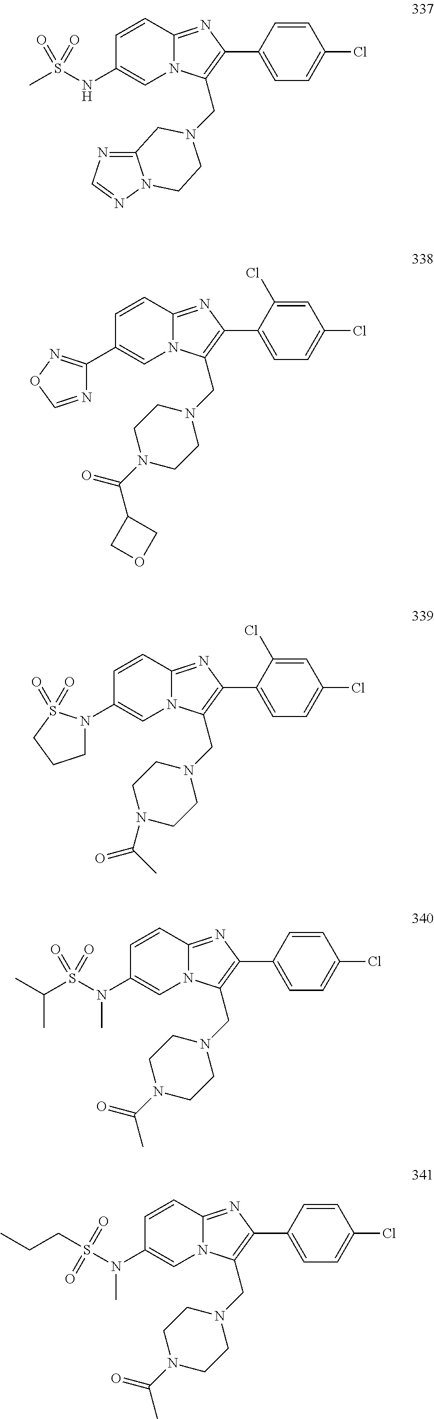 [SMILES: CS(=O)(=O)Nc1ccc2nc(-c3ccc(Cl)cc3)c(CN3CCn4ncnc4C3)n2c1 ; O=C(C1COC1)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(N4CCCS4(=O)=O)cn23)CC1 ; CC(=O)N1CCN(Cc2c(-c3ccc(Cl)cc3)nc3ccc(N(C)S(=O)(=O)C(C)C)cn23)CC1 ; CCCS(=O)(=O)N(C)c1ccc2nc(-c3ccc(Cl)cc3)c(CN3CCN(C(C)=O)CC3)n2c1]

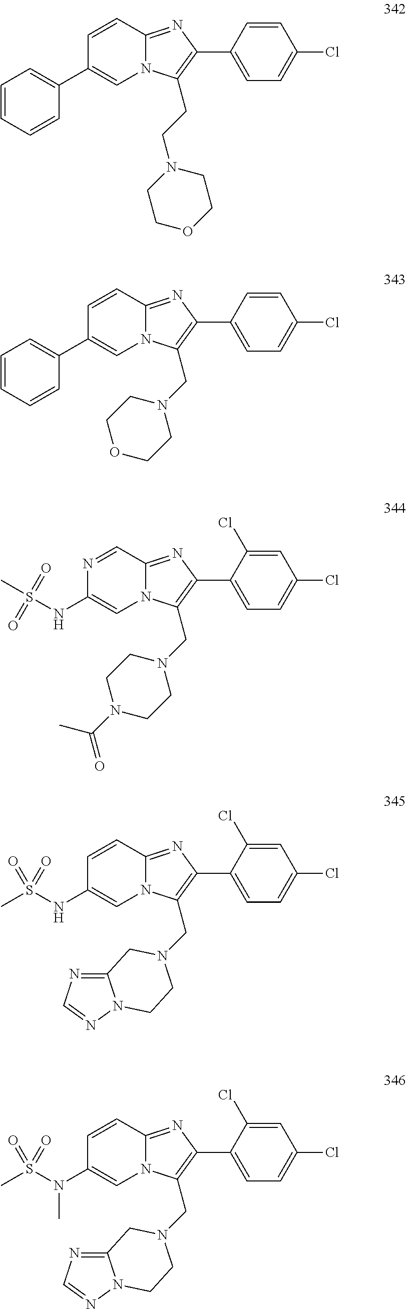 [SMILES: Clc1ccc(-c2nc3ccc(-c4ccccc4)cn3c2CCN2CCOCC2)cc1 ; Clc1ccc(-c2nc3ccc(-c4ccccc4)cn3c2CN2CCOCC2)cc1 ; CC(=O)N1CCN(Cc2c(-c3ccc(Cl)cc3Cl)nc3cnc(NS(C)(=O)=O)cn23)CC1 ; CS(=O)(=O)Nc1ccc2nc(-c3ccc(Cl)cc3Cl)c(CN3CCn4ncnc4C3)n2c1 ; CN(c1ccc2nc(-c3ccc(Cl)cc3Cl)c(CN3CCn4ncnc4C3)n2c1)S(C)(=O)=O]

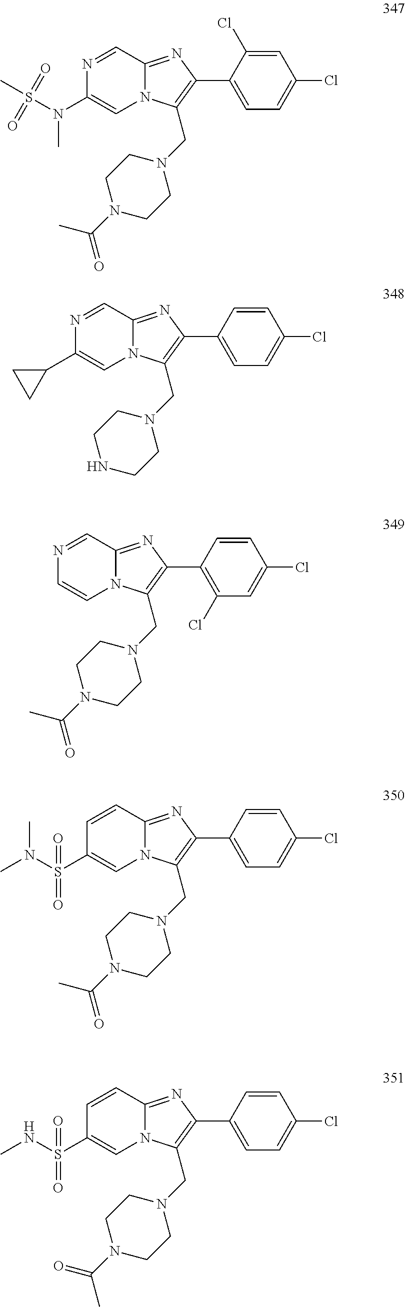

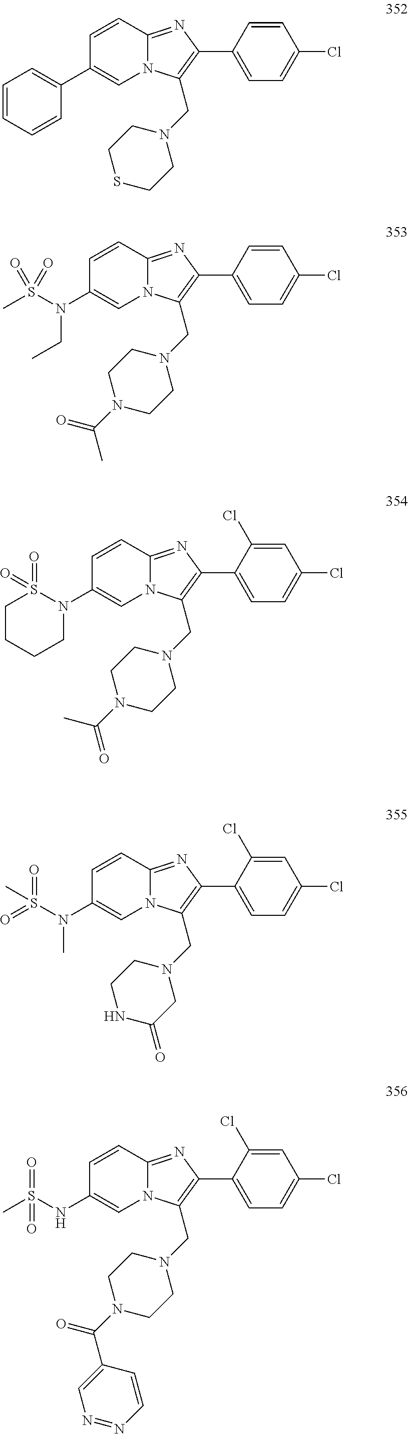 [SMILES: Clc1ccc(-c2nc3ccc(-c4ccccc4)cn3c2CN2CCSCC2)cc1 ; CCN(c1ccc2nc(-c3ccc(Cl)cc3)c(CN3CCN(C(C)=O)CC3)n2c1)S(C)(=O)=O ; CC(=O)N1CCN(Cc2c(-c3ccc(Cl)cc3Cl)nc3ccc(N4CCCCS4(=O)=O)cn23)CC1 ; CN(c1ccc2nc(-c3ccc(Cl)cc3Cl)c(CN3CCNC(=O)C3)n2c1)S(C)(=O)=O ; CS(=O)(=O)Nc1ccc2nc(-c3ccc(Cl)cc3Cl)c(CN3CCN(C(=O)c4ccnnc4)CC3)n2c1]

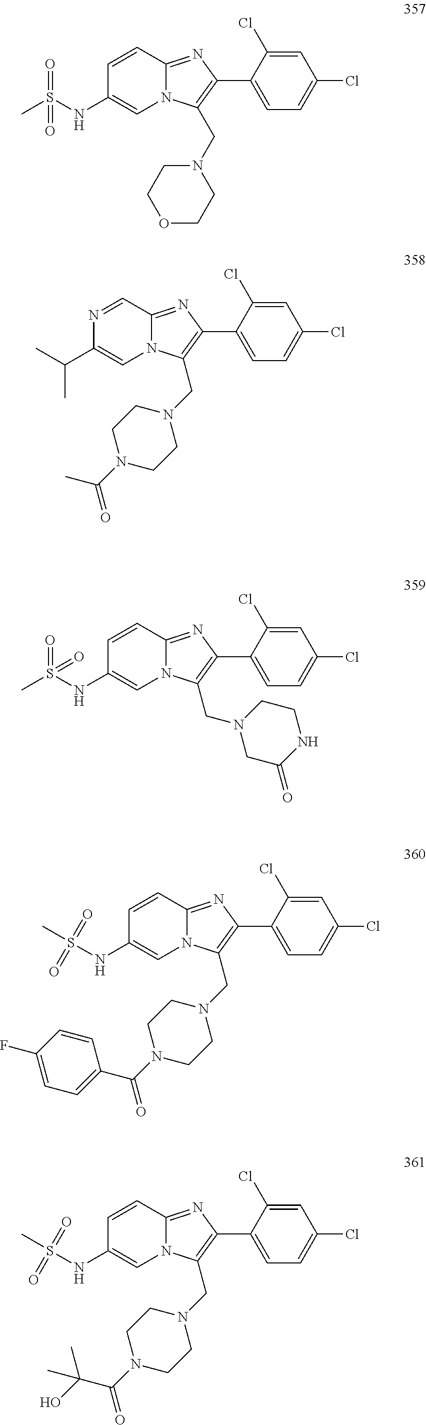 [SMILES: CS(=O)(=O)Nc1ccc2nc(-c3ccc(Cl)cc3Cl)c(CN3CCOCC3)n2c1 ; CC(=O)N1CCN(Cc2c(-c3ccc(Cl)cc3Cl)nc3cnc(C(C)C)cn23)CC1 ; CS(=O)(=O)Nc1ccc2nc(-c3ccc(Cl)cc3Cl)c(CN3CCNC(=O)C3)n2c1 ; CS(=O)(=O)Nc1ccc2nc(-c3ccc(Cl)cc3Cl)c(CN3CCN(C(=O)c4ccc(F)cc4)CC3)n2c1 ; CC(C)(O)C(=O)N1CCN(Cc2c(-c3ccc(Cl)cc3Cl)nc3ccc(NS(C)(=O)=O)cn23)CC1]

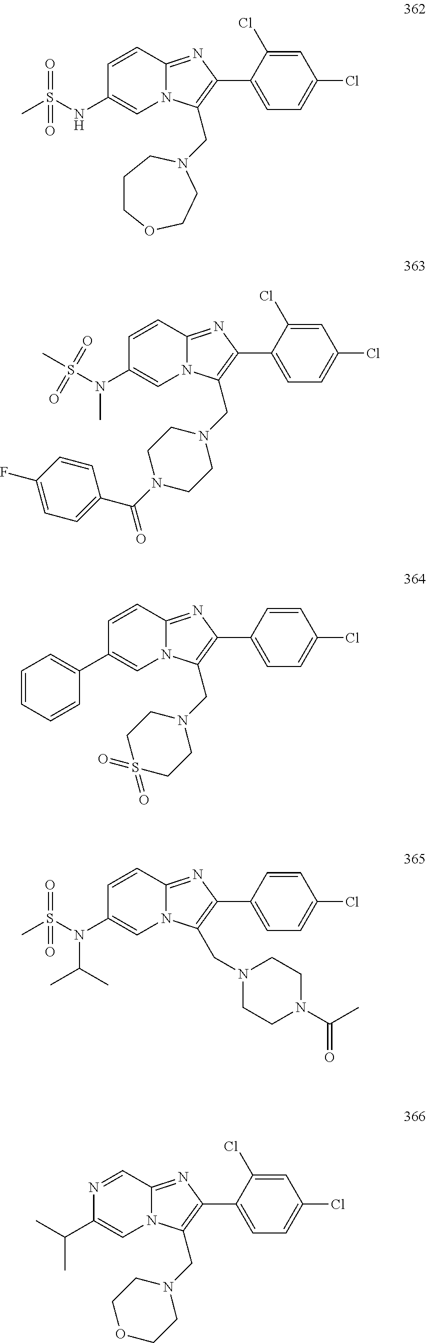

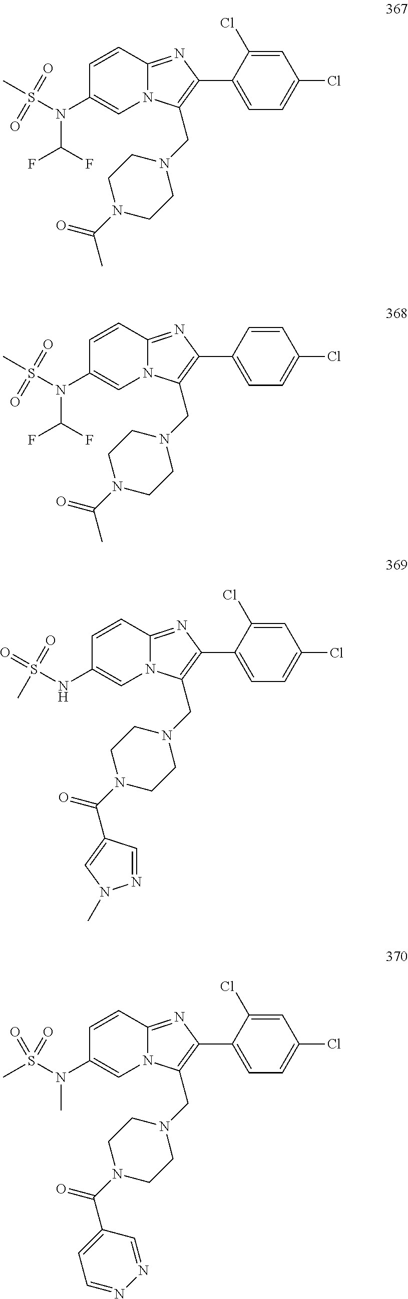 [SMILES: CC(=O)N1CCN(Cc2c(-c3ccc(Cl)cc3Cl)nc3ccc(N(C(F)F)S(C)(=O)=O)cn23)CC1 ; CC(=O)N1CCN(Cc2c(-c3ccc(Cl)cc3)nc3ccc(N(C(F)F)S(C)(=O)=O)cn23)CC1 ; Cn1cc(C(=O)N2CCN(Cc3c(-c4ccc(Cl)cc4Cl)nc4ccc(NS(C)(=O)=O)cn34)CC2)cn1 ; CN(c1ccc2nc(-c3ccc(Cl)cc3Cl)c(CN3CCN(C(=O)c4ccnnc4)CC3)n2c1)S(C)(=O)=O]

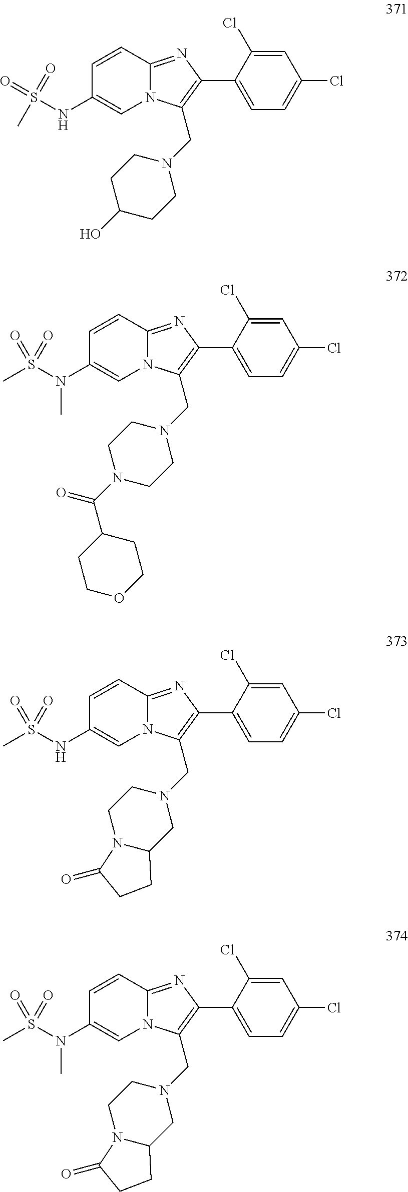 [SMILES: CS(=O)(=O)Nc1ccc2nc(-c3ccc(Cl)cc3Cl)c(CN3CCC(O)CC3)n2c1 ; CN(c1ccc2nc(-c3ccc(Cl)cc3Cl)c(CN3CCN(C(=O)C4CCOCC4)CC3)n2c1)S(C)(=O)=O ; CS(=O)(=O)Nc1ccc2nc(-c3ccc(Cl)cc3Cl)c(CN3CCN4C(=O)CCC4C3)n2c1 ; CN(c1ccc2nc(-c3ccc(Cl)cc3Cl)c(CN3CCN4C(=O)CCC4C3)n2c1)S(C)(=O)=O]

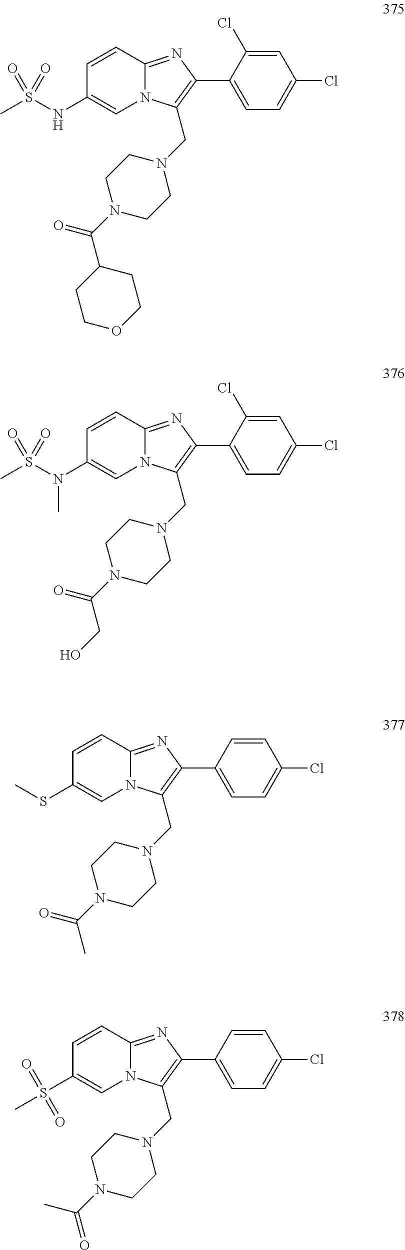 [SMILES: CS(=O)(=O)Nc1ccc2nc(-c3ccc(Cl)cc3Cl)c(CN3CCN(C(=O)C4CCOCC4)CC3)n2c1 ; CN(c1ccc2nc(-c3ccc(Cl)cc3Cl)c(CN3CCN(C(=O)CO)CC3)n2c1)S(C)(=O)=O ; CSc1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(S(C)(=O)=O)cn23)CC1]

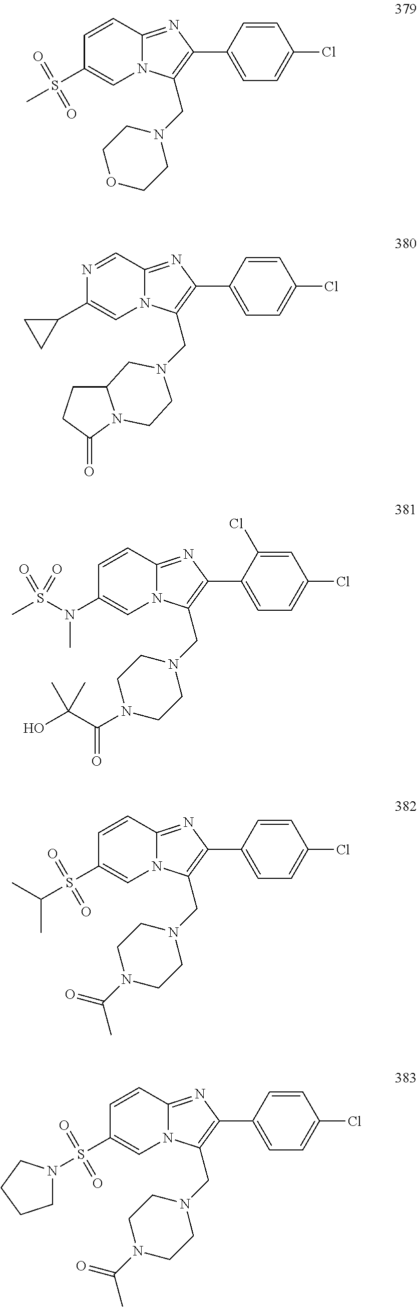 [SMILES: CS(=O)(=O)c1ccc2nc(-c3ccc(Cl)cc3)c(CN3CCOCC3)n2c1 ; O=C1CCC2CN(Cc3c(-c4ccc(Cl)cc4)nc4cnc(C5CC5)cn34)CCN12 ; CN(c1ccc2nc(-c3ccc(Cl)cc3Cl)c(CN3CCN(C(=O)C(C)(C)O)CC3)n2c1)S(C)(=O)=O ; CC(=O)N1CCN(Cc2c(-c3ccc(Cl)cc3)nc3ccc(S(=O)(=O)C(C)C)cn23)CC1 ; CC(=O)N1CCN(Cc2c(-c3ccc(Cl)cc3)nc3ccc(S(=O)(=O)N4CCCC4)cn23)CC1]

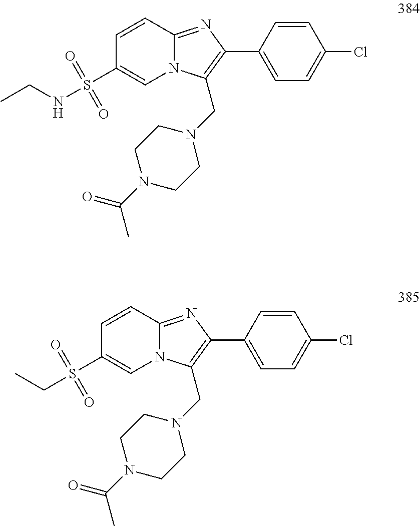 [SMILES: CCNS(=O)(=O)c1ccc2nc(-c3ccc(Cl)cc3)c(CN3CCN(C(C)=O)CC3)n2c1 ; CCS(=O)(=O)c1ccc2nc(-c3ccc(Cl)cc3)c(CN3CCN(C(C)=O)CC3)n2c1]

It is particularly preferred that the method of synthesis of the invention is a method for producing such compounds.

The invention also provides a method for screening for a SLC2A class I transporter inhibitor compound having a structure as defined above, and especially as defined in respect of the compounds new to science, as described above. The method typically comprises:
(a) contacting a receptor having a SLC2A class I transporter function with a test compound having a structure as defined in respect of the compounds, as described above;
(b) measuring the transport of a species across the receptor, which species is one whose transport is facilitated by a SLC2A class I transporter; and
(c) determining whether the test compound is a SLC2A class I transporter inhibitor from the measurements taken in step (b).

In further typical embodiments, the method comprises:
(a) contacting a cell comprising a SLC2A class I transporter with the test compound;
(b) measuring the transport of the species across a membrane of the cell; and
(c) determining whether the test compound is a SLC2A class I transporter inhibitor from the measurements taken in step (b).

The species is not especially limited, provided that it may be transported via a SLC2A class I transporter, and in typical embodiments the species is selected from a substituted or unsubstituted carbohydrate compound such as a substituted or unsubstituted sugar compound, and a mixture of two or more of the above. In more typical embodiments, the species comprises a substituted or unsubstituted glucose. In preferred embodiments, the glucose or other species is labelled, such as with a radiolabel.

When a cell-based assay is employed, the cell is not especially limited provided that it comprises a SLC2A class I transporter. However, in more typical embodiments the cell is a cell that has been transfected such that it comprises a SLC2A class I transporter at its surface.

In typical embodiments, the method may measure the uptake or release of the species from a cell. Known methods and materials for uptake assays may be employed, and the skilled person may select appropriate methods materials and conditions according to the general technical knowledge of such assays.

The invention will now be described in more detail, by way of example only, with reference to the following specific embodiments.

### EXAMPLES

### Example 1

### Methods of Synthesis

### Synthesis 1: 4-((2-(4-chlorophenyl)-6-phenylimidazo[1,2-a]pyridin-3-yl)methyl)morpholine (4)

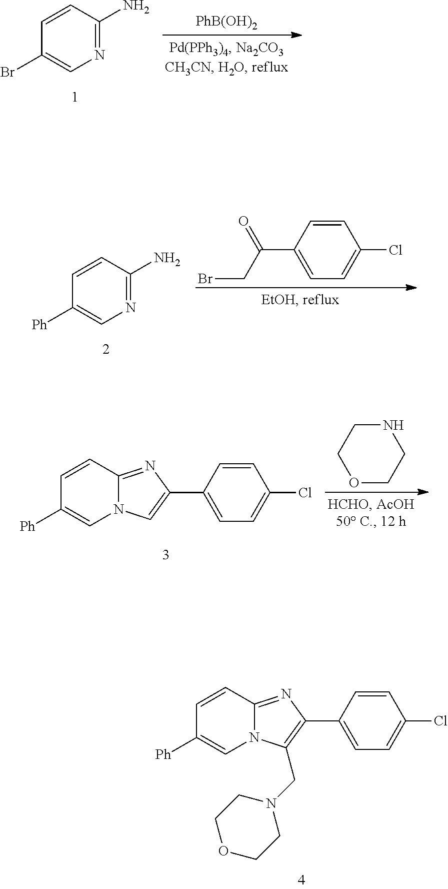

### Preparation of S-phenylpyridin-2-amine (2)

To a stirred solution of 5-bromopyridin-2-amine (1) (5.0 g, 28.9 mmol) in acetonitrile (75 mL) at room temperature was added phenylboronic acid (4.93 g, 40.5 mmol), Na₂CO₃ (4.59 g, 43.4 mmol), H₂O (25 mL) and Pd(PPh₃)₄ (3.30 g, 2.89 mmol). The resulting mixture was heated at 90° C. for 16 h. After cooling to room temperature the reaction mixture was filtered through a pad of celite, and the filtrate was partitioned and extracted with EtOAc (5×100 mL). The combined organic layers were dried over anhydrous Na₂SO₄ and concentrated under reduced pressure. The residue obtained was purified by column chromatography (silica gel, 0 to 20% EtOAc/hexanes) to afford 5-phenylpyridin-2-amine (2) (3.0 g, 61%) as an off-white solid: MS (Multi-mode, ESI/APCI) m/z 172 [M+H]⁺.

### Preparation of 2-(4-chlorophenyl)-6-phenylimidazo[1,2-a]pyridine (3)

To a stirred solution of 5-phenylpyridin-2-amine (2) (300 mg, 1.76 mmol) in EtOH (10 mL) at room temperature was added 4-chlorophenacyl bromide (410 mg, 1.76 mmol). The mixture was stirred at reflux for 16 h. After cooling to room temperature the reaction mixture was concentrated under reduced pressure. The residue was dissolved in CH₂Cl₂ (30 mL), washed with 1N NaOH (5 mL), and the organic layer was dried over Na₂SO₄. The solvent was removed under reduced pressure and the residue purified by column chromatography (silica gel, 0 to 30% EtOAc/hexanes) to afford 2-(4-chlorophenyl)-6-phenylimidazo[1,2-a]pyridine 3 (0.3 g, 56%) as a yellow solid: MS (Multi-mode, ESI/APCI) m/z 305 [M+H]⁺.

### Preparation of 4-((2-(4-chlorophenyl)-6-phenylimidazo[1,2-a]pyridin-3-yl)methyl)morpholine (4)

To a solution of 2-(4-chlorophenyl)-6-phenylimidazo[1,2-a]pyridine (3) (300 mg, 0.98 mmol) in methanol (5 mL) was added morpholine (103 mg, 1.18 mmol), glacial acetic acid (3 ml) and formaldehyde (2 mL). The reaction mixture was heated at reflux for 24 h before it was allowed to cool to room temperature and concentrated under reduced pressure. The residue was dissolved in EtOAc and residual acetic acid quenched with excess saturated NaHCO₃ solution. The organic layer was separated, concentrated under reduced pressure and the crude product purified by flash chromatography (silica gel, 15:85 EtOAc/hexanes) to afford 4-((2-(4-chlorophenyl)-6-phenylimidazo[1,2-a]pyridin-3-yl)methyl)morpholine (4) (60 mg, 15%) as an off-white solid: MS (APCI) m/z 404 [M+H]⁺.

### Synthesis 2: 4-((6-phenyl-2-(4-(trifluoromethyl)phenyl)imidazo[1,2-a]pyridin-3-yl)methyl)morpholine (6)

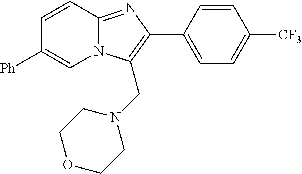 [SMILES: FC(F)(F)c1ccc(-c2nc3ccc(-c4ccccc4)cn3c2CN2CCOCC2)cc1]

### Preparation of 6-phenyl-2-(4-(trifluoromethyl)phenyl)imidazo[1,2-a]pyridine (5)

To a stirred solution of 5-phenylpyridin-2-amine (2) (200 mg, 1.17 mmol) in EtOH (10 mL) at room temperature was added 2-bromo-1-(4-(trifluoromethyl)phenyl)ethanone (312 mg, 1.17 mmol) and Na₂CO₃ (373 mg, 3.52 mmol). The resulting reaction mixture was stirred at reflux for 16 h. After cooling to room temperature the reaction mixture was concentrated and the resulting residue partitioned between EtOAc (20 mL) and water (10 mL). The organic layer was separated and the aqueous phase further extracted with EtOAc (10 mL). The combined organic extracts were dried over Na₂SO₄, filtered and concentrated under reduced pressure. The crude product was purified by column chromatography (silica gel, 0 to 40% EtOAc/hexanes) to afford 6-phenyl-2-(4-(trifluoromethyl)phenyl)imidazo[1,2-a]pyridine (5) (0.3 g, 75%) as a white solid: MS (ESI) m/z 339 [M+H]⁺.

### Preparation of 4-((6-phenyl-2-(4-(trifluoromethyl)phenyl)imidazo[1,2-a]pyridin-3-yl)methyl)morpholine (6)

Aqueous formaldehyde (0.37 mL of 37% wt solution, 4.58 mmol) followed by morpholine (0.8 ml, 9.17 mmol) was added with stirring to glacial acetic acid (2 mL) at 0° C. The mixture was stirred at 0° C. for a further 1 h before addition of 6-phenyl-2-(4-(trifluoromethyl)phenyl)imidazo[1,2-a]pyridine (5) (300 mg, 0.91 mmol) dissolved in CH₂Cl₂ (2 mL) and MeOH (4 mL). The reaction mixture was then heated to 50° C. for 12 h. After cooling the reaction mixture was concentrated under reduced pressure and the residue obtained dissolved in EtOAc before excess acetic acid was quenched with saturated NaHCO₃ solution. The organic layer was separated, concentrated under reduced pressure and the crude product purified by column chromatography (silica gel, 0 to 50% EtOAc/hexanes) to afford 4-((6-phenyl-2-(4-(trifluoromethyl)phenyl)imidazo[1,2-a]pyridin-3-yl)methyl)morpholine (6) (167 mg, 41%) as an off-white solid: MS (APCI) m/z 438 [M+H]⁺.

### Synthesis 3: 4-((2-(4-chlorophenyl)-6-(2-fluorophenyl)imidazo[1,2-a]pyridin-3-yl)methyl)morpholine (9)

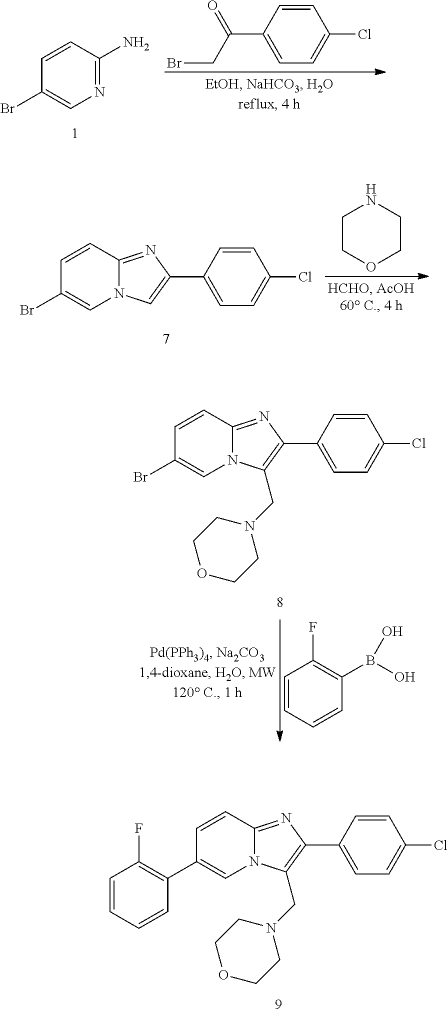

### Preparation of 6-bromo-2-(4-chlorophenyl)imidazo[1,2-a]pyridine (7)

To a stirred solution of 5-bromopyridin-2-amine (1) (30.0 g, 173.4 mmol) in EtOH (240 mL) and water (60 mL) at room temperature was added 4-chlorophenacyl bromide (45.5 g, 190.7 mmol) and NaHCO₃ (14.5 g, 173.4 mmol). The resulting reaction mixture was stirred at reflux for 4 h. After cooling to room temperature the resulting solid was filtered and washed with MTBE to give 6-bromo-2-(4-chlorophenyl)imidazo[1,2-a]pyridine (7) (40 g, 75%) as an off-white solid. MS (Multi-mode, ESI/APCI) m/z 307 [M+H]⁺.

### Preparation of 4-((6-bromo-2-(4-chlorophenyl)imidazo[1,2-a]pyridin-3-yl)methyl)morpholine (8)

To a stirred solution of 6-bromo-2-(4-chlorophenyl)imidazo[1,2-a]pyridine (7) (40.0 g, 130.3 mmol) in acetic acid (150 mL) at room temperature was added aqueous formaldehyde (0.37 mL of 38% wt solution, 6 mL, 195.4 mmol) and morpholine (12.4 mL, 143.3 mmol).

The resulting reaction mixture was stirred at 60° C. for 4 h. After cooling to room temperature the reaction mixture was quenched with 6N NaOH solution and extracted with EtOAc (3×100 mL). The combined organic layers were dried over anhydrous Na₂SO₄ and concentrated under reduced pressure. The residue was washed with MTBE to afford 4-((6-bromo-2-(4-chlorophenyl)imidazo[1,2-a]pyridin-3-yl)methyl)morpholine (8) (20.0 g, 37%) as an off-white solid: MS (Multi-mode, ESI/APCI) m/z 406 [M+H]⁺.

### Preparation of 4-((2-(4-chlorophenyl)-6-(2-fluorophenyl)imidazo[1,2-a]pyridin-3-yl)methyl)morpholine (9)

To a stirred solution of 4-((6-bromo-2-(4-chlorophenyl)imidazo[1,2-a]pyridin-3-yl)methyl)morpholine (8) (200 mg, 0.49 mmol), 2-fluorophenylboronic acid (38 mg, 0.54 mmol) and Na₂CO₃ (103 mg, 0.98 mmol) water and dioxane (2:10 v/v, 15 mL). After bubbling nitrogen through the reaction mixture for 15 min, Pd(PPh₃)₄ (56 mg, 1.1 mmol) was added and the reaction mixture was heated at 120° C. for 60 min in a microwave reactor. After cooling to room temperature, the reaction mixture was diluted with water and the resulting solid was filtered and purified by combiflash column chromatography (silica, 0 to 40% EtOAc/hexanes) to afford 4-((2-(4-chlorophenyl)-6-(2-fluorophenyl)imidazo[1,2-a]pyridin-3-yl)methyl)morpholine (9) (40 mg, 20%) as a white solid: MS (Multi-mode, ESI/APCI) m/z 422 [M+H]⁺.

### Synthesis 4: (4-((2-(4-chlorophenyl)-6-phenylimidazo[1,2-a]pyridin-3-yl)methyl)piperazin-1-yl)cyclopropyl)methanone (13)

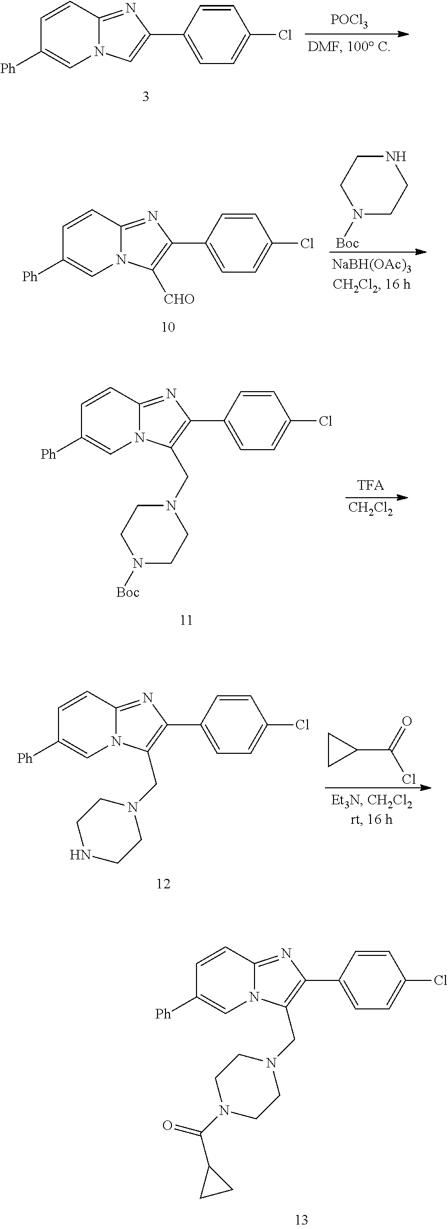

### Preparation of 2-(4-chlorophenyl)-6-phenylimidazo[1,2-a]pyridine-3-carbaldehyde (10)

A stirred solution of 2-(4-chlorophenyl)-6-phenylimidazo[1,2-a]pyridine (3) (1.0 g, 3.20 mmol) in DMF (10 mL) at room temperature was added with POCl₃ (0.76 mL, 8.00 mmol). The resulting reaction mixture was stirred at 90° C. for 16 h. The reaction mixture was cooled to 0° C. and quenched with 0.5 M Na₂CO₃. The resulting solid was filtered, washed with water and dried over Na₂SO₄ to afford 2-(4-chlorophenyl)-6-phenylimidazo[1,2-a]pyridine-3-carbaldehyde (10) (0.7 g, 64%) as an off-white solid: MS (Multi-mode, ESI/APCI) m/z 333 [M+H]⁺.

### Preparation of tert-butyl 4-((2-(4-chlorophenyl)-6-phenylimidazo[1,2-a]pyridin-3-yl)methyl)piperazine-1-carboxylate (11)

To a stirred solution of 2-(4-chlorophenyl)-6-phenylimidazo[1,2-a]pyridine-3-carbaldehyde (10) (0.5 g, 1.50 mmol) in CH₂Cl₂ (10 mL) at room temperature was added with N-Boc-piperazine (0.3 g, 1.65 mmol). The mixture was stirred at room temperature for 1 h before Na(OAc)₃BH was added and stirring continued for 16 h. The reaction mixture was quenched with saturated NaHCO₃ solution and extracted with CH₂Cl₂ (3×00 mL). The combined organic layers were dried over anhydrous Na₂SO₄ and concentrated under reduced pressure. The residue obtained was purified by column chromatography (silica gel, 0 to 40% EtOAc/hexanes) to afford tert-butyl 4-((2-(4-chlorophenyl)-6-phenylimidazo[1,2-a]pyridin-3-yl)methyl)piperazine-1-carboxylate (11) (0.2 g, 26%) as an off-white solid. MS (Multi-mode, ESI/APCI) m/z 503 [M+H]⁺.

### Preparation of 2-(4-chlorophenyl)-6-phenyl-3-(piperazin-1-ylmethyl)imidazo[1,2-a]pyridine (12)

A stirred solution of tert-butyl 4-((2-(4-chlorophenyl)-6-phenylimidazo[1,2-a]pyridin-3-yl)methyl)piperazine-1-carboxylate (11) (2.2 g, 4.37 mmol) in CH₂Cl₂ (50 mL) at room temperature was added trifluoroacetic acid (5 mL). The resulting reaction mixture was stirred at room temperature for 16 h then quenched with saturated NaHCO₃ solution and extracted with CH₂Cl₂(3×200 mL). The combined organic layers were dried over anhydrous Na₂SO₄ and concentrated under reduced pressure. The residue obtained was purified by column chromatography (silica gel, 0 to 10% MeOH/CH₂Cl₂) to afford 2-(4-chlorophenyl)-6-phenyl-3-(piperazin-1-ylmethyl)imidazo[1,2-a]pyridine (12) (1.4 g, 82%) as an off-white solid. MS (Multi-mode, ESI/APCI) m/z 403 [M+H]⁺.

### Preparation of (4-((2-(4-chlorophenyl)-6-phenylimidazo[1,2-a]pyridin-3-yl)methyl)piperazin-1-yl)(cyclopropyl)methanone (13)

To a stirred solution of 2-(4-chlorophenyl)-6-phenyl-3-(piperazin-1-ylmethyl)imidazo[1,2-a]pyridine (12) (0.2 g, 0.49 mmol) in CH₂Cl₂ (10 mL) at room temperature was added Et₃N (0.1 g, 0.99 mmol) and cyclopropanoyl chloride (62 mg, 0.59 mmol). The resulting reaction mixture was stirred at room temperature for 16 h then concentrated under reduced pressure. The residue obtained was purified by column chromatography (silica gel, 0 to 30% EtOAc/hexanes) to afford (4-((2-(4-chlorophenyl)-6-phenylimidazo[1,2-a]pyridin-3-yl)methyl)piperazin-1-yl)(cyclopropyl)methanone (13) (40 mg, 18%) as an off-white solid. MS (Multi-mode, ESI/APCI) m/z 471 [M+H]⁺.

### Synthesis 5: (4-((2-(4-chlorophenyl)-6-phenylimidazo[1,2-a]pyridin-3-yl)methyl)piperazin-1-yl)(pyridin-4-yl)methanone (14)

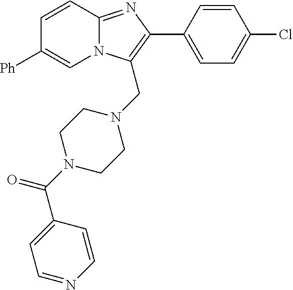 [SMILES: O=C(c1ccncc1)N1CCN(Cc2c(-c3ccc(Cl)cc3)nc3ccc(-c4ccccc4)cn23)CC1]

To a stirred solution of pyridine-4-carboxylic acid (0.092 g, 0.74 mmol) in DMF (10 mL) at room temperature was added HATU (0.37 g, 0.99 mmol) and DIPEA (0.19 g, 1.49 mmol). The resulting reaction mixture was stirred at room temperature for 1 h before addition of 2-(4-chlorophenyl)-6-phenyl-3-(piperazin-1-ylmethyl)imidazo[1,2-a]pyridine (12) and stirring continued for 4 h. The reaction mixture was quenched with water (10 mL) and the resulting solid was filtered and purified by column chromatography (silica gel, 0 to 60% EtOAc/hexanes) to afford (4-((2-(4-chlorophenyl)-6-phenylimidazo[1,2-a]pyridin-3-yl)methyl)piperazin-1-yl)(pyridin-4-yl)methanone (14) (45 mg, 18%) as an off-white solid. MS (Multi-mode, ESI/APCI) m/z 508 [M+H]⁺.

### Example 2

### Glucose Uptake Assays and pIC₅₀ Measurements for Test Compounds

HEK293 cells were engineered to stably express either human GLUT1, GLUT2, GLUt3 or GLUT4 by transfection with the pReceiver-Lv105 plasmid, containing either hGLUT1, hGLUT2, hGLUT3, or hGLUT4 gene constructs with lipofectamine 2000, hGLUT1- hGLUT2- hGLUT3- or hGLUT4-overexpressing polyclonal populations were isolated under purinomycin selective pressure (1 μg/ml) and maintained in Dulbecco's modified Eagle's medium supplemented with 1 μg/ml purinomycin+10% foetal bovine serum (FBS).

60,000 cells per well were seeded overnight into poly-D-lysine coasted 96-well plates. Following media removal, cells were washed twice in warm glucose-free uptake buffer (25 mM HEPES/KOH pH 7.4, 125 mM NaCl, 4.8 mM KCl, 1.2 mM MgSO₄, 1.2 mM CaCl₂, 1.2 mM KH₂PO₄). 90 μl of test compounds were added in glucose-free uptake buffer and cells were incubated at 37° C. for 30 min. 10 μl of [3H]-deoxy-D-glucose (0.25 μCi, 125 μM final assay concentration) was then added per well in glucose-free uptake buffer and uptake was allowed to proceed at 37° C. for 30 min.

Cells were then washed twice with ice-cold PBS and 100 μl of Microscint-20 was added per well. Glucose uptake was quantified with a scintillation counter. The half maximal inhibitory concentration (IC₅₀) (a measure of the effectiveness of the test compound in inhibiting glucose uptake) was determined. The pIC50 values for a variety of test compounds are shown in Table 2.

TABLE 2
pIC50 values for GLUT1, GLUT2, GLUT3 and GLUT4
inhibition determined for test compounds

| | GLUT1 | GLUT2 | GLUT3 | GLUT4 |
|---|---|---|---|---|
| Compound | pIC₅₀ | pIC₅₀ | pIC₅₀ | pIC₅₀ |
| 1 | ++ | NT | NT | NT |
| 3 | ++ | NT | NT | NT |
| 4 | + | NT | NT | NT |
| 5 | + | NT | NT | NT |
| 6 | +− | NT | NT | NT |
| 7 | + | NT | NT | NT |
| 8 | +++ | +++ | +++ | +++ |
| 9 | ++ | NT | NT | NT |
| 11 | + | NT | NT | NT |
| 12 | + | NT | NT | NT |
| 13 | +++ | NT | NT | NT |
| 15 | ++ | NT | NT | NT |
| 16 | ++ | NT | NT | NT |
| 17 | +− | NT | NT | NT |
| 18 | +++ | NT | NT | NT |
| 19 | +++ | NT | NT | NT |
| 20 | +++ | NT | NT | NT |
| 21 | ++ | NT | NT | NT |
| 22 | +++ | NT | NT | NT |
| 24 | +++ | NT | NT | NT |
| 25 | ++ | NT | NT | NT |
| 26 | ++ | NT | NT | NT |
| 27 | ++ | NT | NT | NT |
| 28 | ++ | NT | NT | NT |
| 29 | + | NT | NT | NT |
| 30 | +− | NT | NT | NT |
| 32 | + | NT | NT | NT |
| 33 | ++ | NT | NT | NT |
| 34 | ++ | NT | NT | NT |
| 35 | +++ | NT | NT | NT |
| 36 | +++ | NT | NT | NT |
| 38 | ++ | NT | NT | NT |
| 39 | + | NT | NT | NT |
| 40 | ++ | NT | NT | NT |
| 41 | ++ | NT | NT | NT |
| 42 | + | NT | NT | NT |
| 43 | +++ | NT | NT | NT |
| 44 | +++ | NT | NT | NT |
| 45 | +++ | NT | NT | NT |
| 46 | ++ | NT | NT | NT |
| 47 | +++ | NT | NT | NT |
| 49 | +++ | NT | NT | NT |
| 50 | +++ | NT | NT | NT |
| 51 | ++ | NT | NT | NT |
| 52 | +++ | NT | NT | NT |
| 53 | +++ | NT | NT | NT |
| 54 | ++ | NT | NT | NT |
| 55 | ++ | NT | NT | NT |
| 56 | ++ | NT | NT | NT |
| 57 | ++ | NT | NT | NT |
| 58 | + | NT | NT | NT |
| 59 | ++ | NT | NT | NT |
| 60 | + | NT | NT | NT |
| 61 | +++ | NT | NT | NT |
| 62 | +++ | NT | NT | NT |
| 63 | ++ | NT | NT | NT |
| 64 | + | NT | NT | NT |
| 65 | ++ | NT | NT | NT |
| 66 | ++ | NT | NT | NT |
| 67 | +++ | NT | NT | NT |
| 68 | + | NT | NT | NT |
| 69 | +++ | NT | NT | NT |
| 70 | +++ | NT | NT | NT |
| 71 | +++ | NT | NT | NT |
| 72 | +− | NT | NT | NT |
| 73 | + | NT | NT | NT |
| 75 | +++ | NT | NT | NT |
| 76 | + | NT | NT | NT |
| 77 | +− | NT | NT | NT |
| 78 | +− | NT | NT | NT |
| 79 | +− | +− | + | ++ |
| 80 | +++ | NT | +++ | +++ |
| 81 | + | NT | NT | NT |
| 82 | ++ | NT | NT | NT |
| 83 | ++ | NT | NT | NT |
| 84 | ++ | NT | NT | NT |
| 85 | ++ | NT | NT | NT |
| 86 | + | NT | NT | NT |
| 87 | ++ | NT | NT | NT |
| 88 | +− | NT | NT | NT |
| 89 | + | NT | NT | NT |
| 90 | +− | NT | NT | NT |
| 91 | +++ | +++ | NT | NT |
| 92 | +− | NT | NT | NT |
| 94 | + | NT | NT | NT |
| 95 | + | NT | NT | NT |
| 97 | + | NT | NT | NT |
| 98 | + | NT | NT | NT |
| 100 | + | NT | NT | NT |
| 101 | ++ | NT | NT | NT |
| 102 | ++ | NT | NT | NT |
| 103 | + | NT | NT | NT |
| 104 | + | NT | NT | NT |
| 105 | ++ | NT | NT | NT |
| 106 | +− | NT | NT | NT |
| 107 | ++ | NT | NT | NT |
| 109 | +− | NT | NT | NT |
| 110 | + | NT | NT | NT |
| 111 | +− | NT | NT | NT |
| 112 | ++ | NT | NT | NT |
| 113 | +− | NT | NT | NT |
| 114 | ++ | NT | NT | NT |
| 115 | +++ | NT | NT | NT |
| 116 | +++ | NT | +++ | +++ |
| 117 | + | NT | NT | NT |
| 118 | + | NT | NT | NT |
| 119 | ++ | NT | NT | NT |
| 120 | ++ | NT | NT | NT |
| 121 | +− | NT | NT | NT |
| 122 | ++ | NT | NT | NT |
| 124 | + | NT | NT | NT |
| 125 | + | NT | NT | NT |
| 126 | +++ | +++ | NT | NT |
| 127 | +− | +− | NT | NT |
| 128 | +++ | NT | NT | NT |
| 129 | +++ | NT | NT | NT |
| 130 | ++ | NT | NT | NT |
| 131 | + | NT | NT | NT |
| 132 | +++ | NT | NT | NT |
| 133 | +++ | NT | NT | NT |
| 134 | ++ | NT | NT | NT |
| 139 | + | NT | NT | NT |
| 141 | ++ | NT | NT | NT |
| 143 | +++ | +++ | +++ | +++ |
| 144 | +++ | NT | NT | NT |
| 145 | +++ | NT | NT | NT |
| 152 | +++ | NT | NT | NT |
| 153 | +− | NT | NT | NT |
| 154 | + | NT | NT | NT |
| 155 | +++ | NT | NT | NT |
| 157 | ++ | NT | NT | NT |
| 158 | + | NT | NT | NT |
| 159 | +++ | NT | NT | NT |
| 162 | + | NT | NT | NT |
| 163 | + | NT | NT | NT |
| 164 | +++ | +++ | +++ | +++ |
| 166 | +++ | NT | +++ | +++ |
| 167 | +++ | NT | NT | NT |
| 168 | +++ | NT | NT | NT |
| 169 | ++ | NT | NT | NT |
| 170 | +++ | NT | +++ | +++ |
| 171 | +++ | +++ | NT | NT |
| 172 | ++ | NT | NT | NT |
| 173 | ++ | NT | NT | NT |
| 174 | ++ | NT | NT | NT |
| 175 | ++ | NT | NT | NT |
| 176 | ++ | NT | NT | NT |
| 177 | + | NT | NT | NT |
| 178 | +++ | NT | NT | NT |
| 179 | +− | NT | NT | NT |
| 180 | +− | NT | NT | NT |
| 181 | +++ | NT | NT | NT |
| 182 | +− | NT | NT | NT |
| 183 | +++ | NT | NT | NT |
| 184 | +++ | NT | NT | NT |
| 185 | ++ | NT | NT | NT |
| 186 | +++ | NT | NT | NT |
| 187 | +++ | +++ | +++ | +++ |
| 188 | ++ | NT | NT | NT |
| 189 | +++ | NT | NT | NT |
| 190 | +++ | NT | NT | NT |
| 191 | +++ | NT | NT | NT |
| 192 | ++ | NT | NT | NT |
| 193 | +++ | NT | NT | NT |
| 194 | ++ | NT | NT | NT |
| 195 | ++ | NT | NT | NT |
| 196 | +++ | NT | NT | NT |
| 197 | + | NT | NT | NT |
| 198 | + | NT | NT | NT |
| 199 | ++ | NT | NT | NT |
| 200 | +++ | NT | NT | NT |
| 201 | +++ | NT | NT | NT |
| 202 | +++ | NT | NT | NT |
| 203 | ++ | NT | NT | NT |
| 204 | +− | NT | NT | NT |
| 205 | ++ | NT | NT | NT |
| 206 | ++ | NT | NT | NT |
| 207 | +++ | NT | NT | NT |
| 208 | +− | NT | NT | NT |
| 209 | +− | NT | NT | NT |
| 210 | ++ | NT | NT | NT |
| 211 | +− | NT | NT | NT |
| 212 | +++ | NT | NT | NT |
| 213 | +++ | NT | NT | NT |
| 214 | +++ | +++ | +++ | +++ |
| 215 | ++ | NT | NT | NT |
| 216 | +++ | NT | NT | NT |
| 217 | +++ | +++ | NT | NT |
| 218 | +++ | NT | NT | NT |
| 219 | +++ | NT | NT | NT |
| 220 | +++ | NT | NT | NT |
| 221 | +++ | NT | NT | NT |
| 222 | +++ | NT | NT | NT |
| 223 | +− | NT | NT | NT |
| 224 | +++ | +++ | NT | NT |
| 225 | +++ | NT | NT | NT |
| 226 | +++ | NT | NT | NT |
| 227 | +++ | +++ | NT | NT |
| 228 | +− | NT | NT | NT |
| 229 | +++ | NT | NT | NT |
| 230 | +++ | NT | NT | NT |
| 231 | +++ | NT | NT | NT |
| 232 | +++ | +++ | NT | NT |
| 233 | +++ | NT | NT | NT |
| 234 | +++ | NT | NT | NT |
| 235 | +++ | NT | NT | NT |
| 236 | +++ | +++ | NT | NT |
| 237 | ++ | NT | NT | NT |
| 238 | +++ | +++ | +++ | +++ |
| 239 | +++ | NT | NT | NT |
| 240 | ++ | NT | NT | NT |
| 241 | ++ | NT | NT | NT |
| 242 | +++ | NT | NT | NT |
| 243 | +++ | NT | NT | NT |
| 244 | +++ | NT | NT | NT |
| 245 | +− | NT | NT | NT |
| 246 | ++ | NT | NT | NT |
| 247 | +++ | +++ | NT | NT |
| 248 | ++ | NT | NT | NT |
| 249 | ++ | NT | NT | NT |
| 250 | +++ | NT | NT | NT |
| 251 | ++ | NT | NT | NT |
| 252 | +++ | ++ | +++ | +++ |
| 253 | +++ | +++ | NT | NT |
| 254 | +++ | NT | NT | NT |
| 255 | +++ | NT | NT | NT |
| 256 | +++ | NT | NT | NT |
| 257 | +++ | NT | NT | NT |
| 258 | +++ | ++ | NT | NT |
| 259 | +++ | ++ | NT | NT |
| 260 | +++ | NT | NT | NT |
| 261 | +++ | +++ | NT | NT |
| 262 | +++ | ++ | NT | NT |
| 263 | +++ | NT | NT | NT |
| 264 | +++ | NT | NT | NT |
| 265 | +++ | +++ | +++ | +++ |
| 266 | +++ | NT | NT | NT |
| 267 | +++ | NT | NT | NT |
| 268 | +++ | NT | NT | NT |
| 269 | +++ | ++ | NT | NT |
| 270 | +++ | NT | NT | NT |
| 271 | +++ | +++ | +++ | +++ |
| 272 | +++ | +++ | NT | NT |
| 273 | +++ | +++ | +++ | +++ |
| 274 | +++ | NT | NT | NT |
| 275 | ++ | NT | NT | NT |
| 276 | +− | NT | NT | NT |
| 277 | +++ | NT | NT | NT |
| 278 | ++ | ++ | NT | NT |
| 279 | +++ | NT | NT | NT |
| 280 | +− | NT | NT | NT |
| 281 | +++ | NT | NT | NT |
| 282 | +− | NT | NT | NT |
| 283 | +− | NT | NT | NT |
| 284 | +++ | NT | NT | NT |
| 285 | +++ | NT | NT | NT |
| 286 | +++ | NT | NT | NT |
| 287 | +++ | NT | NT | NT |
| 288 | +++ | NT | NT | NT |
| 289 | +++ | NT | NT | NT |
| 290 | +− | NT | NT | NT |
| 291 | +− | NT | NT | NT |
| 292 | +++ | ++ | NT | NT |
| 293 | +++ | +++ | NT | NT |
| 294 | +++ | +++ | NT | NT |
| 295 | +++ | +++ | NT | NT |
| 296 | +++ | +++ | +++ | +++ |
| 297 | +++ | +++ | +++ | +++ |
| 298 | ++ | +− | NT | NT |
| 299 | +++ | +++ | NT | NT |
| 300 | +++ | +++ | NT | NT |
| 301 | +− | ++ | NT | NT |
| 302 | +− | ++ | NT | NT |
| 303 | +++ | ++ | +++ | +++ |
| 304 | ++ | +− | NT | NT |
| 305 | +− | +− | NT | NT |
| 306 | +++ | +++ | +++ | +++ |
| 307 | +++ | ++ | NT | NT |
| 308 | +++ | +++ | NT | NT |
| 309 | +++ | ++ | +++ | +++ |
| 310 | +++ | ++ | +++ | +++ |
| 311 | +++ | +++ | +++ | +++ |
| 312 | +++ | +++ | NT | NT |
| 313 | +++ | ++ | +++ | +++ |
| 314 | +++ | ++ | +++ | +++ |
| 315 | ++ | +− | NT | NT |
| 316 | +++ | ++ | +++ | +++ |
| 317 | ++ | ++ | NT | NT |
| 318 | +++ | ++ | NT | NT |
| 319 | +++ | ++ | NT | NT |
| 320 | +++ | +++ | NT | NT |
| 321 | +++ | +++ | +++ | +++ |
| 322 | +++ | +++ | +++ | +++ |
| 323 | +++ | +++ | NT | NT |
| 324 | +++ | ++ | NT | NT |
| 325 | +++ | ++ | NT | NT |
| 326 | +++ | +++ | +++ | +++ |
| 327 | +++ | ++ | NT | NT |
| 328 | ++ | ++ | NT | NT |
| 329 | +++ | +++ | NT | NT |
| 330 | +++ | ++ | NT | NT |
| 331 | +− | +− | NT | NT |
| 332 | +++ | ++ | +++ | +++ |
| 333 | +++ | +++ | +++ | +++ |
| 334 | +++ | ++ | +++ | +++ |
| 335 | +++ | +++ | +++ | +++ |
| 336 | +++ | ++ | +++ | +++ |
| 337 | +++ | ++ | NT | NT |
| 338 | +++ | +++ | NT | NT |
| 339 | +++ | +++ | +++ | +++ |
| 340 | +++ | ++ | +++ | +++ |
| 341 | +++ | +− | +++ | +++ |
| 342 | +− | ++ | NT | NT |
| 343 | +− | +− | NT | NT |
| 344 | +− | +− | NT | NT |
| 345 | +++ | ++ | NT | NT |
| 346 | +++ | ++ | +++ | +++ |
| 347 | +++ | ++ | NT | NT |
| 348 | +− | +− | NT | NT |
| 349 | ++ | +− | NT | NT |
| 350 | +++ | ++ | +++ | +++ |
| 351 | +++ | ++ | +++ | +++ |
| 352 | +− | +− | NT | NT |
| 353 | +++ | ++ | +++ | +++ |
| 354 | +++ | +++ | NT | NT |
| 355 | +++ | +++ | +++ | +++ |
| 356 | +++ | +++ | +++ | +++ |
| 357 | +++ | ++ | NT | NT |
| 358 | +++ | +++ | NT | NT |
| 359 | +++ | ++ | NT | NT |
| 360 | +++ | ++ | +++ | +++ |
| 361 | +++ | ++ | +++ | +++ |
| 362 | +++ | ++ | NT | NT |
| 363 | +++ | +++ | +++ | +++ |
| 364 | ++ | +− | NT | NT |
| 365 | +++ | ++ | +++ | +++ |
| 366 | +++ | +++ | +++ | +++ |
| 367 | +++ | +++ | +++ | +++ |
| 368 | +++ | +++ | +++ | +++ |
| 369 | +++ | ++ | +++ | +++ |
| 370 | +++ | +++ | +++ | +++ |
| 371 | +++ | ++ | +++ | +++ |
| 372 | +++ | +++ | +++ | +++ |
| 373 | +++ | +++ | +++ | +++ |
| 374 | +++ | +++ | +++ | +++ |
| 375 | +++ | ++ | +++ | +++ |
| 376 | +++ | +++ | +++ | +++ |
| 377 | ++ | ++ | +++ | +++ |
| 378 | +++ | +− | +++ | +++ |
| 379 | ++ | +− | NT | NT |
| 380 | +++ | +++ | +++ | +++ |
| 381 | +++ | ++ | +++ | +++ |
| 382 | +++ | ++ | +++ | +++ |
| 383 | +++ | ++ | NT | NT |
| 384 | +++ | ++ | NT | NT |
| 385 | +++ | ++ | +++ | +++ |

Key:
+++ = pIC₅₀ ≧ 6.00
++ = pIC₅₀ 5.00-5.99
+ = pIC₅₀ 4.00-4.99
+− = pIC₅₀ < 4.00
NT = not tested

The Table shows that a large number of the test compounds show strong SLC2A class I transporter (all of GLUT1, GLUT2, GLUT3 and GLUT4) inhibitory function. It is notable that there is strong correlation between GLUT1, GLUT2, GLUT3 and GLUT4 activity, demonstrating the close relationship between the different SLC2A transporters within class I.

### Example 3

### Inhibition of Glucose Uptake in Cells Using Compound 155 (See FIG. 2)

### [3H]-Deoxy-D-Glucose Uptake Assay

Glucose uptake assays were performed in accordance with the protocol of Example 2, using increasing concentrations of test compound (compound 155). The results are shown in FIG. 2 and Table 3.

### Lactate Production Assay

A549 cells were grown in DMEM/F12K+L-Glutamine supplemented with 10% foetal bovine serum. Cells were re-suspended in assay media (DMEM without phenol red, pyruvate and glucose, 10% foetal bovine serum, 2 mM L-Glutamine and 5 or 17 mM glucose) and seeded at 40×10³ cells per well into 96-well plates and incubated overnight. Increasing concentrations of the test compound (compound 155) and vehicle control were added and incubated for 4 hours at 37° C., 5% CO₂. Lactate reagent was added (Trinity Biotech), the plates were incubated in the dark for 7 min at room temperature before capturing the absorbance at 540 nm using a plate reader.

FIG. 2 shows: (A) inhibition of [3H]-deoxy-D-glucose uptake; (B,C) inhibition of lactate secretion

### Example 4

### Cell Proliferation and Apoptosis Assays Using Compound 155 (See FIG. 3)

### Cell Growth/Apoptosis Assay

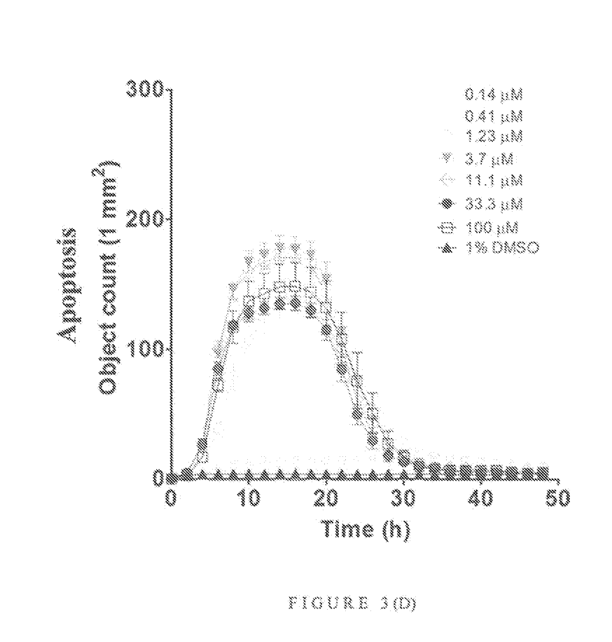

A549 adenocarcinoma NucLight Red cells were grown in Ham's F12K medium supplemented with 10% foetal bovine serum, 2 mM Glutamax, 1% pen/strep and 0.5 μg ml-l puromycin. Cells were seeded at 1×10⁴ cells per well into 384-well microtitre-plates with media containing 5 mM (FIGS. 3A and 3C) or 17 mM glucose (FIGS. 3B and 3D). The cells were left to adhere before the addition of increasing concentration of test compound (compound 155) and vehicle control. The cells stably expressed Essen CellPlayer NucLight Red Fluorescent Protein to allow measurement of cell proliferation (FIGS. 3 A and B). The culture medium contained Essen CellPlayer 96-well Kinetic Caspase 3/7-reagent and Biotium DEVD-NucView 488 to allow measurement of apoptosis induction (FIGS. 3 C and D). Cells were monitored continuously for 48 hours of compound exposure using Essen IncuCyte.

pIC50 values for the proliferation and apoptosis assays were derived from area under the curve (AUC) analysis of data from the first 24 hours of compound exposure. The results are shown in Table 3.

TABLE 3
Compound Activity Determination

| Assay | pIC₅₀ |
|---|---|
| Glucose Uptake | 7.59 |
| Lactate Secretion (5 mM | 7.11 |
| glucose) | |
| Lactate Secretion (17 mM | 6.40 |
| glucose) | |
| Proliferation (5 mM glucose) | >6.90 |
| Proliferation (17 mM glucose) | 6.42 |
| Apoptosis (5 mM glucose) | 6.50 |
| Apoptosis (17 mM glucose) | 6.09 |

These studies demonstrate that the compounds of the invention are effective inhibitors in real cell systems, and are consequently effective at reducing or preventing cell proliferation and/or at increasing or promoting apoptosis in cells. Thus, the inhibitory effect of the compounds translates into significant biological activity. The utility of the compounds as therapeutics, especially for treating cancer and other conditions and disorders where apoptosis and proliferation control are useful, will be apparent.

1-7. (canceled)

8. An SLC2A class I transporter inhibitor compound for use in medicine, which compound comprises a formula selected from one of the following: 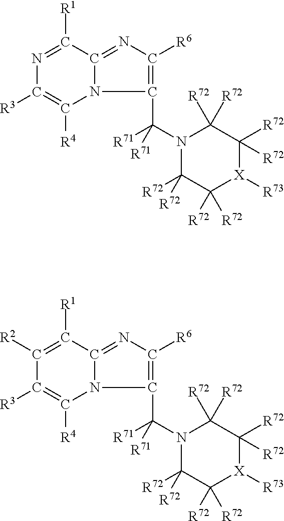
wherein R¹, R², R³, R⁴ and R⁶ may be the same or different and are independently selected from H and a substituted or unsubstituted organic group; X is independently selected from N, O and S; each R⁷¹ and each R⁷² may be the same or different and are independently selected from H and a substituted or unsubstituted organic group; two R⁷¹ groups and/or two R⁷² groups may together form a carbonyl group; R⁷³ is absent when its X atom is O or S and may be the same or different as R⁷¹ and R⁷² and is independently selected from H and a substituted or unsubstituted organic group; and wherein an R⁷² and R⁷³ may together form a substituted or unsubstituted saturated or unsaturated aliphatic or aromatic homocyclic or heterocyclic ring.

9. A compound for use in medicine according to claim 8, which compound comprises a formula selected from one of the following: 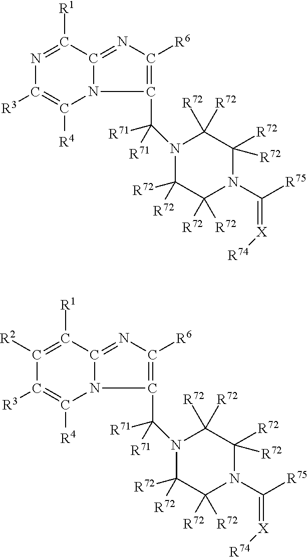
wherein X is independently selected from N, O and S; R⁷⁴ is absent when X is O or S and is selected from H and a substituted or unsubstituted organic group when X is N; and R⁷⁵ is selected from H and a substituted or unsubstituted organic group; preferably wherein X is O and/or R⁷⁵ is a substituted or unsubstituted group selected from a linear or branched alkyl group, and an aliphatic or aromatic saturated or unsaturated homocyclic or heterocyclic ring such as a cycloalkyl group, a saturated or unsaturated heterocyclic group, and an aryl group; and/or preferably wherein R¹ is H, R² is H, R⁴ is H or Me, R³ is not H, R⁶ is not H, each R⁷¹ is H, and each R⁷² is H.

10. A compound for use in medicine according to claim 8, which compound comprises a formula selected from one of the following: 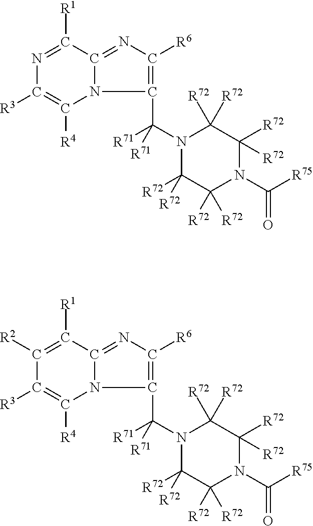
wherein R⁷⁵ is a substituted or unsubstituted organic group selected from: a linear or branched alkyl group, a cycloalkyl group, a saturated or unsaturated heterocyclic group, and an aryl group; preferably wherein R¹ is H, R² is H, R⁴ is H or Me, R³ is not H, R⁶ is not H, each R⁷¹ is H, each R⁷² is H;
preferably wherein R⁷⁵ is a group having the following structure: 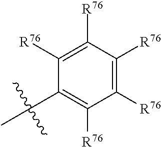
wherein each R⁷⁶ may be the same or different and is independently selected from H and a substituted or unsubstituted organic group; preferably wherein R¹ is H, R² is H, R⁴ is H or Me, R³ is not H, R⁶ is not H, each R⁷¹ is H, each R⁷² is H, and at least one of R⁷⁶ is not H.

11. A compound for use in medicine according to claim 8, wherein R³ is H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl); preferably a C₃-C₆ alkyl group (such as propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R³ is selected from the following:
an unsubstituted, 2-monosubstituted, or 2,6-disubstituted phenyl group, preferably where the substituent is selected from F, Cl and CN (such as Ph-, 2-F-Ph-, 2,6-F₂-Ph-, 2-C₁-Ph-, 2,6-Cl₂-Ph-, 2-CN-Ph-, 2,6-(CN)₂-Ph-, 2,6-F, CN-Ph-, 2,6-F, Cl-Ph-, and 2,6-Cl, CN-Ph-);
an unsubstituted or 3-monosubstituted pyridine-2-yl, or a 2,6-disubstituted pyridine-4-yl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂—Pyr-4-yl;
an unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl);
a substituted or unsubstituted 1,2,4-oxadiazol-3-yl group;
a cyclic aminosulphonyl- group (such as —N(SO₂)(CH₂)₃ and —N(SO₂)(CH₂)₄);
a linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe;
a linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl);
a linear or branched sulphonyl group (such as —SO₂Me, —SO₂Et, —SO₂Pr, —SO₂iPr);
a thioether group (such as —SMe, —SEt, —SPr, and SiPr); and
an isopropyl, cyclopropyl and a propen-2-yl group;

12. A compound for use in medicine according to claim 8, wherein R⁶ is H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl),
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂-NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R⁶ comprises—an aromatic group selected from Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-,
particularly preferably wherein R⁶ is selected from the following:
an unsubstituted, 2-, 3- or 4-monosubstituted, and a 2,4 or 3,4-disubstituted phenyl group, preferably wherein the substituent is F, Cl or —OMe (such as 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph);
a substituted or unsubstituted pyridine-2-yl, pyridine-3-yl or pyridine-4-yl group preferably wherein the substituent is F or CI (such as pyridine-2-yl, pyridine-3-yl, pyridine-4-yl, 3-F-pyridine-2-yl, 3-Cl-pyridine-2-yl, 4-F-pyridine-2-yl, 4-Cl-pyridine-2-yl, 5-F-pyridine-2-yl, 5-Cl-pyridine-2-yl; and
a cyclic ether group (such as tetrahydrofuran-2-yl, tetrahydrofuran-3-yl, terahydropyran-2-yl, tetrahydropyran-3-yl and tetrahydropyran-4-yl).

13. A compound for use in medicine according to claim 8, wherein the group: 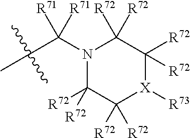
is selected from the following groups: 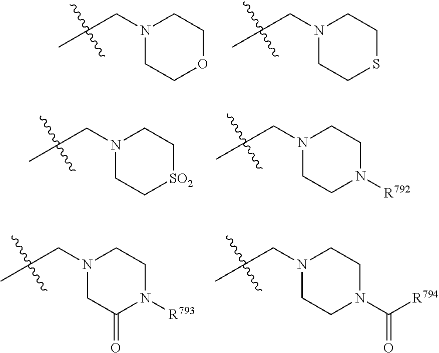
and also from a group having the following structure: 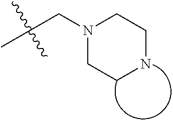 [SMILES: *CN1CCNCC1]
where the ring fused to the piperazine ring is a 5- or 6-membered ring optionally having 1 or more further heteroatoms in the ring selected from N and O and optionally having one or more substituents, preferably wherein this group is selected from the following: 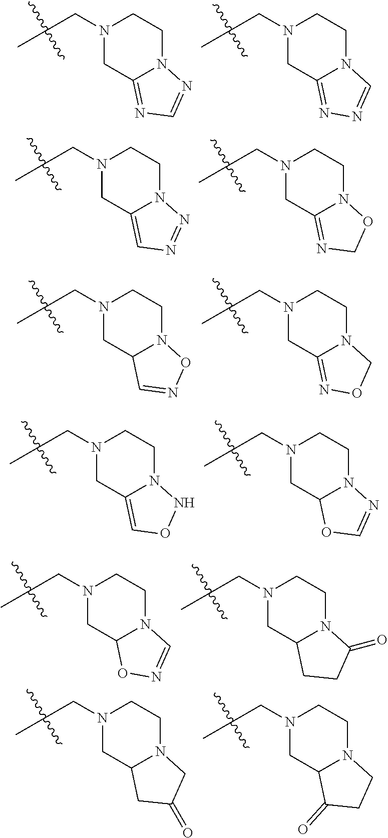
wherein R⁷⁹², R⁷⁹³, and R⁷⁹⁴ are selected from the following:
R⁷⁹² is selected from H, Me, Et, Pr, iPr, cyPr, cyBu, —(CO)-Me, —(CO)-Et, —(CO)—Pr, —(CO)iPr, —SO₂Me, and SO₂Et, preferably H, Me, —SO₂Me and —(CO)-Me;
R⁷⁹³ is selected from H, Me, Et, Pr, iPr, cyPr, cyBu, —(CO)-Me, —(CO)Et, —(CO)Pr, and —(CO)iPr, preferably H, Me and —(CO)-Me;
R⁷⁹⁴ is selected from H, Me, Et, Pr, iPr, cyPr, cyBu; an unsubstituted, 2-, 3- or 4-monosubstituted, and a 2,4 or 3,4-disubstituted phenyl group, preferably wherein the substituent is F, ClCN, or —OMe (such as 2-F-Ph, 2-Cl-Ph, 2-CN-Ph, 2-OMe-Ph, 3-F-Ph, 3-Cl-Ph, 3-CN-Ph, 3-OMe-Ph, 4-F-Ph, 4-Cl-Ph, 4-CN-Ph, 4-OMe-Ph, 2,4-F₂-Ph, 2,4-Cl₂-Ph, 2,4-(CN)₂-Ph, 2,4-(OMe)₂-Ph, 3,4-F₂-Ph, 3,4-Cl₂-Ph, 3,4-(CN)₂-Ph, and 3,4-(OMe)₂-Ph); —NH₂, —NHMe, and —NMe₂; —OH, —OMe, —OEt, —OPr, —OiPr, —OnBu, and —OtBu; —CH₂OH, —CHMeOH, —C(Me)₂OH, —CH₂OMe, —CHMeOMe, —C(Me)₂OMe, —CH₂OEt, —CHMeOEt, and —C(Me)₂OEt; and a heterocyclic ring group having from 4-7 ring atoms with at least one heteroatom selected from N, O, and S, which ring group may be saturated or unsaturated and may be substituted or unsubstituted, preferably wherein the heterocyclic ring group is selected from the following groups:

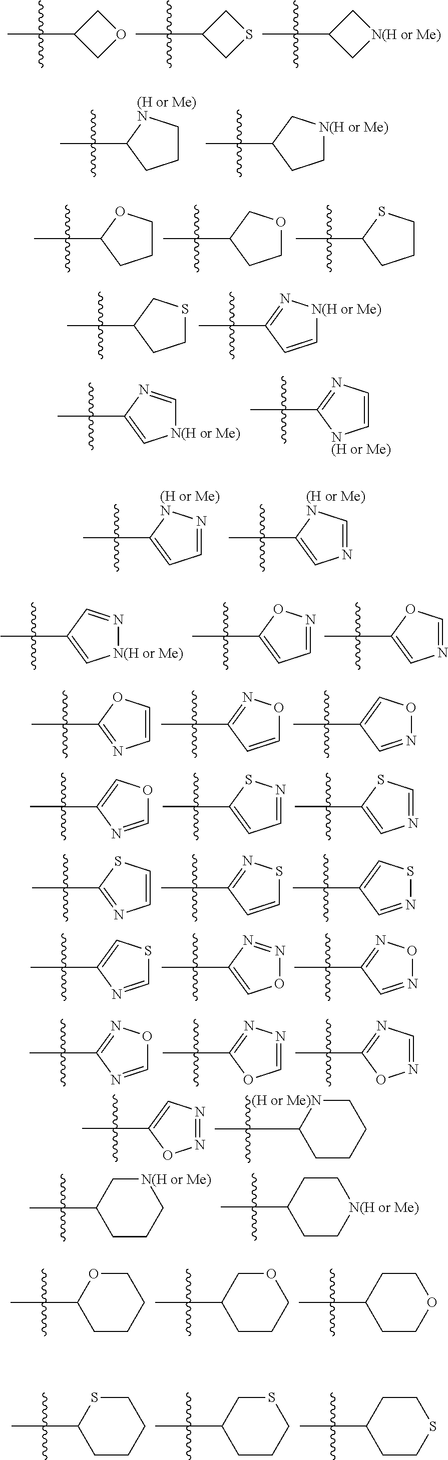

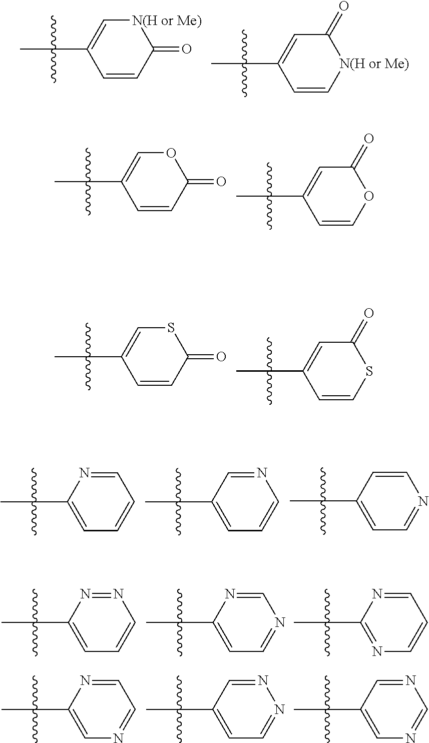

14. A compound for use in medicine according to claim 8, wherein R¹, R², and R⁴ are each independently H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

15. A compound for use in medicine according to any claim 8, wherein each of R⁷¹, R⁷², R⁷³, R⁷⁴, R⁷⁵ and R⁷⁶ is independently H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂-NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R⁷⁵ is a substituted or unsubstituted group selected from a linear or branched alkyl group, a cycloalkyl group, a saturated or unsaturated heterocyclic group including an aromatic heterocyclic group, and an aryl group; chosen from the following:
a linear or branched C₁-C₆ alkyl group (such as Me, Et, Pr, i-Pr, n-Bu, i-Bu, t-Bu, pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, and —Cl₃);
a linear or branched primary secondary or tertiary C₁-C₆ alkylamine group (such as —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ alkoxyalkyl or aryloxyalkyl group (such as —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
wherein two of R⁷¹ and/or two of R⁷² when attached to the same carbon atom may together form a ketone group.

16. A compound for use in medicine according to claim 8,
wherein:
R³ is selected from the following:
an unsubstituted, 2-monosubstituted, or 2,6-disubstituted phenyl group, preferably where the substituent is selected from F, Cl and CN (such as Ph-, 2-F-Ph-, 2,6-F₂-Ph-, 2-C₁-Ph-, 2,6-Cl₂-Ph-, 2-CN-Ph-, 2,6-(CN)₂-Ph-, 2,6-F, CN-Ph-, 2,6-F, Cl-Ph-, and 2,6-Cl, CN-Ph-);
an unsubstituted or 3-monosubstituted pyridine-2-yl, or a 2,6-disubstituted pyridine-4-yl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂-Pyr-4-yl. In this context Pyr means pyridine;
an unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl);
a substituted or unsubstituted 1,2,4-oxadiazol-3-yl group;
a cyclic aminosulphonyl- group (such as —N(SO₂)(CH₂)₃ and —N(SO₂)(CH₂)₄);
a linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe;
a linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl);
a linear or branched sulphonyl group (such as —SO₂Me, —SO₂Et, —SO₂Pr, —SO₂iPr);
a thioether group (such as —SMe, —SEt, —SPr, and SiPr);
a branched or cyclic alkyl or alkenyl group having 3 carbon atoms or more, preferably having from 3 to 6 carbon atoms, and preferably selected from an isopropyl, a cyclopropyl and a propen-2-yl group;
R⁶ is selected from the following:
an unsubstituted, 2-, 3- or 4-monosubstituted, and a 2,4 or 3,4-disubstituted phenyl group, preferably wherein the substituent is F, Cl or —OMe (such as 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph);
a substituted or unsubstituted pyridine-2-yl, pyridine-3-yl or pyridine-4-yl group preferably wherein the substituent is F or CI (such as pyridine-2-yl, pyridine-3-yl, pyridine-4-yl, 3-F-pyridine-2-yl, 3-Cl-pyridine-2-yl, 4-F-pyridine-2-yl, 4-Cl-pyridine-2-yl, 5-F-pyridine-2-yl, 5-Cl-pyridine-2-yl;
a cyclic ether group (such as tetrahydrofuran-2-yl, tetrahydrofuran-3-yl, terahydropyran-2-yl, tetrahydropyran-3-yl and tetrahydropyran-4-yl), and;
the group: 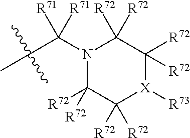
is selected from the following groups: 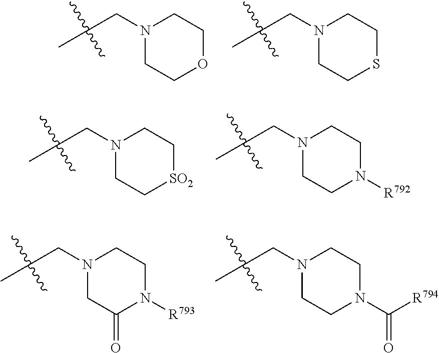
and also from a group having the following structure: 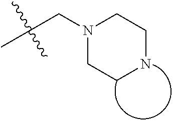 [SMILES: *CN1CCNCC1]
where the ring fused to the piperazine ring is a 5- or 6-membered ring optionally having 1 or more further heteroatoms in the ring selected from N and O and optionally having one or more substituents, preferably wherein this group is selected from the following: 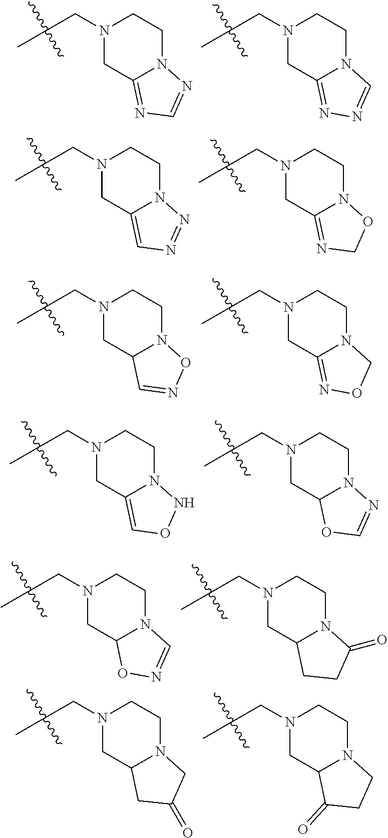
wherein R⁷⁹², R⁷⁹³, and R⁷⁹⁴ and R are as defined below:
R⁷⁹² is selected from H, Me, Et, Pr, iPr, cyPr, cyBu, —(CO)-Me, —(CO)-Et, —(CO)—Pr, —(CO)iPr, —SO₂Me, and SO₂Et, preferably H, Me, —SO₂Me and —(CO)-Me;
R⁷⁹³ is selected from H, Me, Et, Pr, iPr, cyPr, cyBu, —(CO)-Me, —(CO)Et, —(CO)Pr, and —(CO)iPr, preferably H, Me and —(CO)-Me;
R⁷⁹⁴ is selected from H, Me, Et, Pr, iPr, cyPr, cyBu; an unsubstituted, 2-, 3- or 4-monosubstituted, and a 2,4 or 3,4-disubstituted phenyl group, preferably wherein the substituent is F, ClCN, or —OMe (such as 2-F-Ph, 2-Cl-Ph, 2-CN-Ph, 2-OMe-Ph, 3-F-Ph, 3-Cl-Ph, 3-CN-Ph, 3-OMe-Ph, 4-F-Ph, 4-Cl-Ph, 4-CN-Ph, 4-OMe-Ph, 2,4-F₂-Ph, 2,4-Cl₂-Ph, 2,4-(CN)₂-Ph, 2,4-(OMe)₂-Ph, 3,4-F₂-Ph, 3,4-Cl₂-Ph, 3,4-(CN)₂-Ph, and 3,4-(OMe)₂-Ph); —NH₂, —NHMe, and —NMe₂; —OH, —OMe, —OEt, —OPr, —OiPr, —OnBu, and —OtBu; —CH₂OH, —CHMeOH, —C(Me)₂OH, —CH₂OMe, —CHMeOMe, —C(Me)₂OMe, —CH₂OEt, —CHMeOEt, and —C(Me)₂OEt; and a heterocyclic ring group having from 4-7 ring atoms with at least one heteroatom selected from N, O, and S, which ring group may be saturated or unsaturated and may be substituted or unsubstituted, preferably wherein the heterocyclic ring group is selected from the following groups:

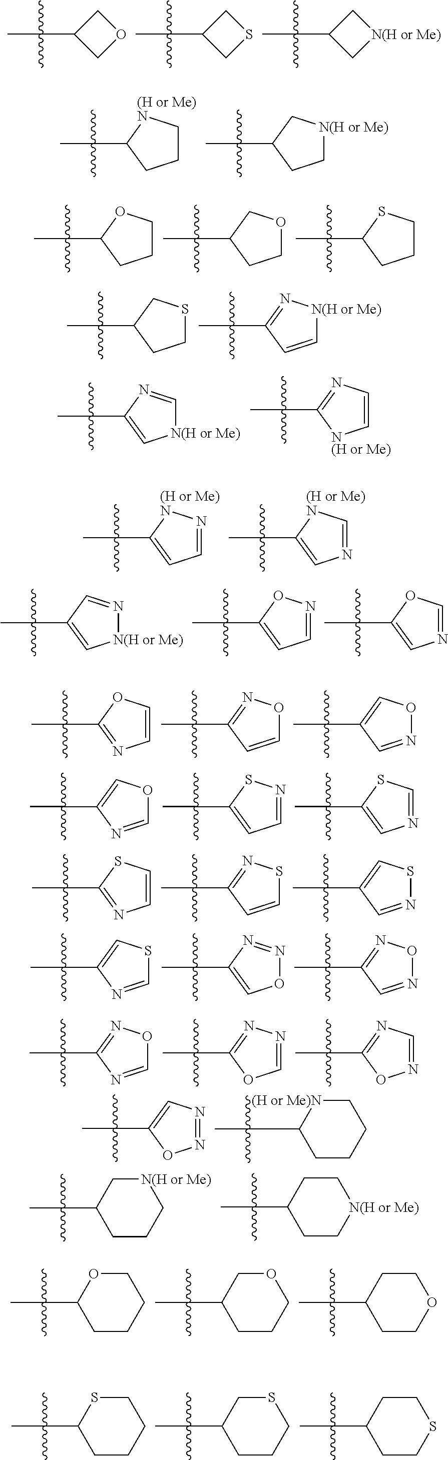

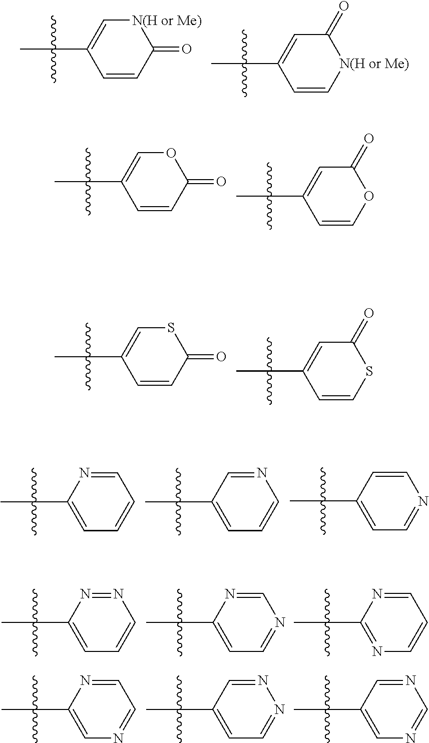

17. A compound for use in medicine according to claim 8, which compound comprises a formula selected from one of the following:

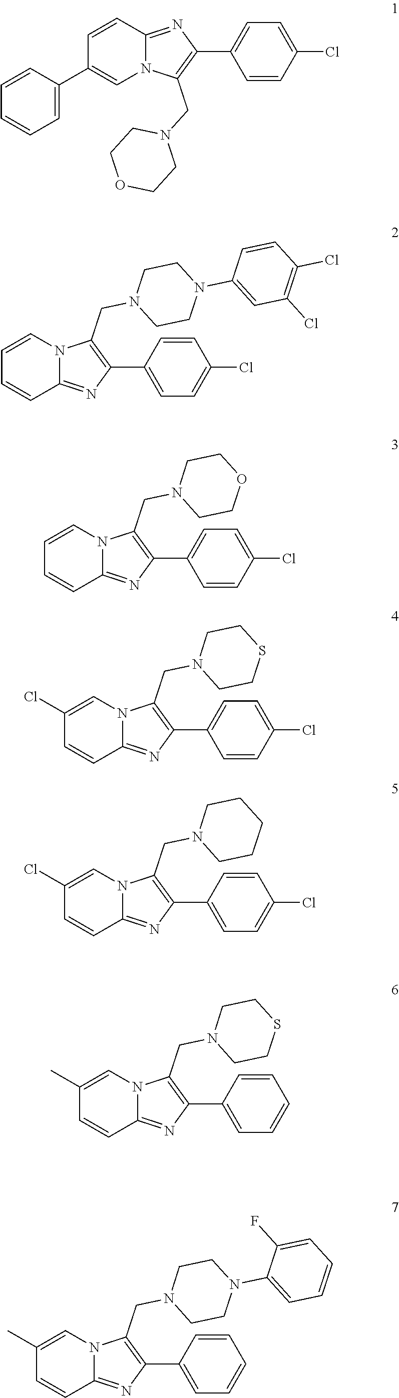

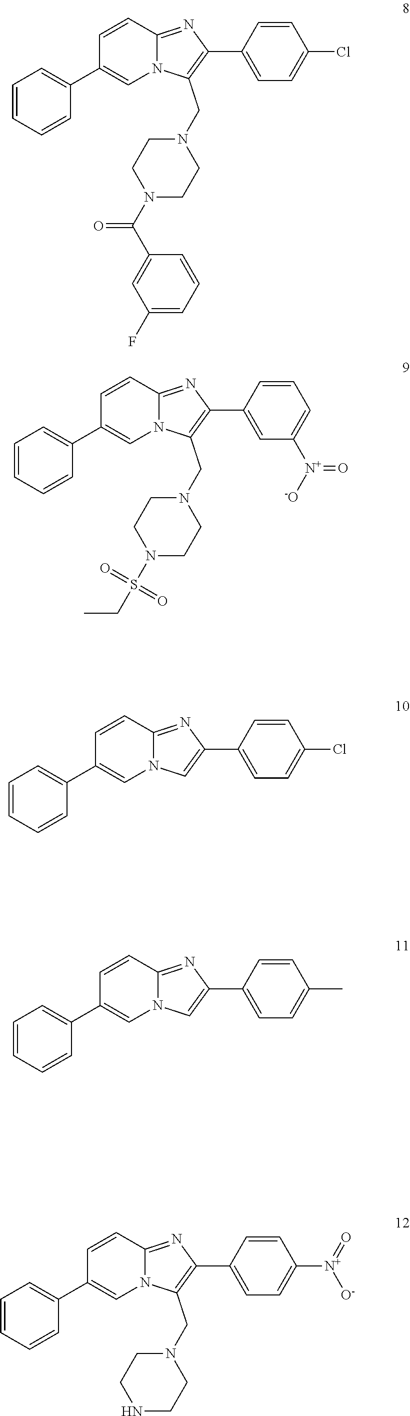

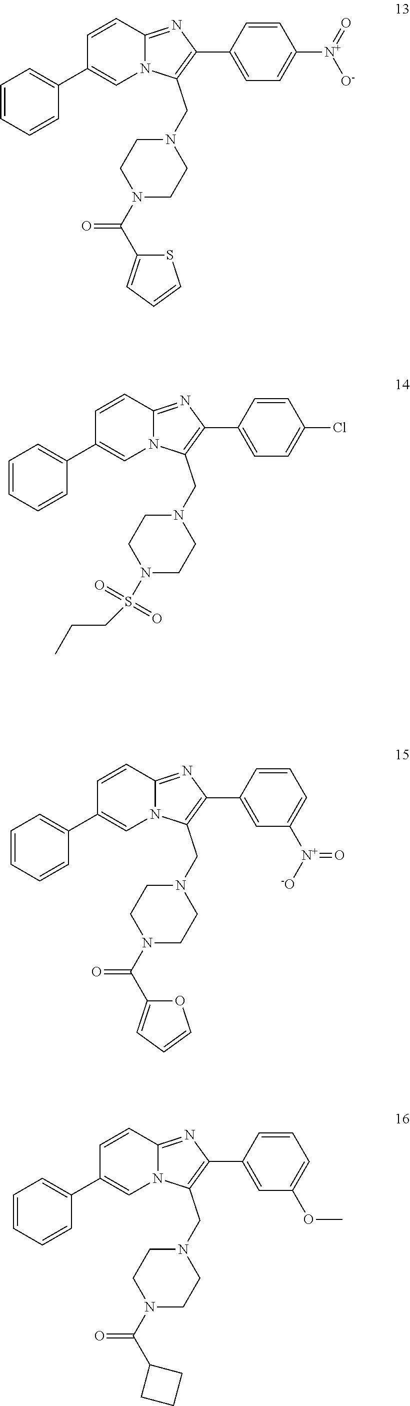

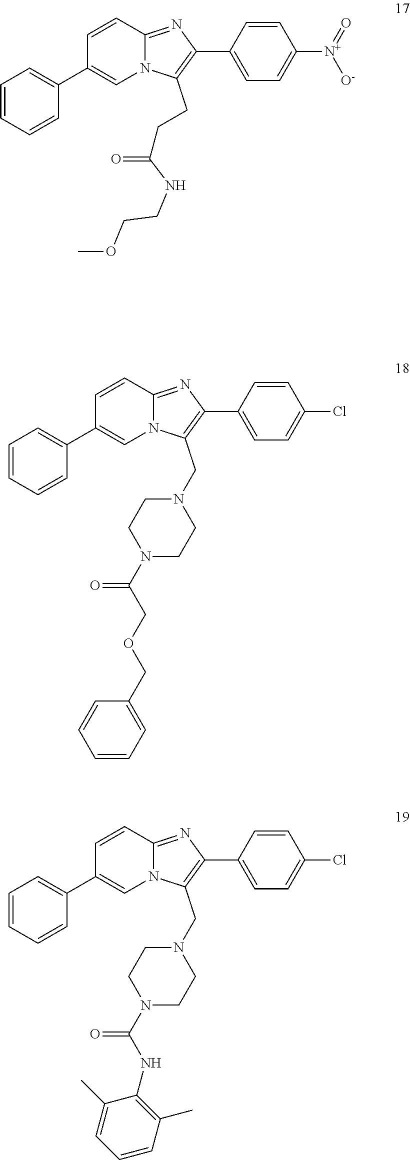 [SMILES: COCCNC(=O)CCc1c(-c2ccc([N+](=O)[O-])cc2)nc2ccc(-c3ccccc3)cn12 ; O=C(COCc1ccccc1)N1CCN(Cc2c(-c3ccc(Cl)cc3)nc3ccc(-c4ccccc4)cn23)CC1 ; Cc1cccc(C)c1NC(=O)N1CCN(Cc2c(-c3ccc(Cl)cc3)nc3ccc(-c4ccccc4)cn23)CC1]

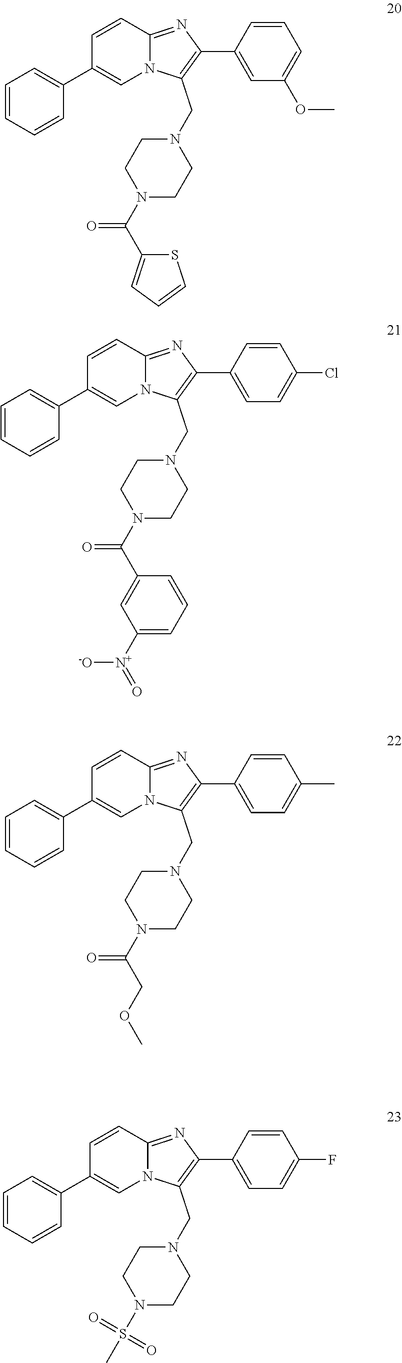

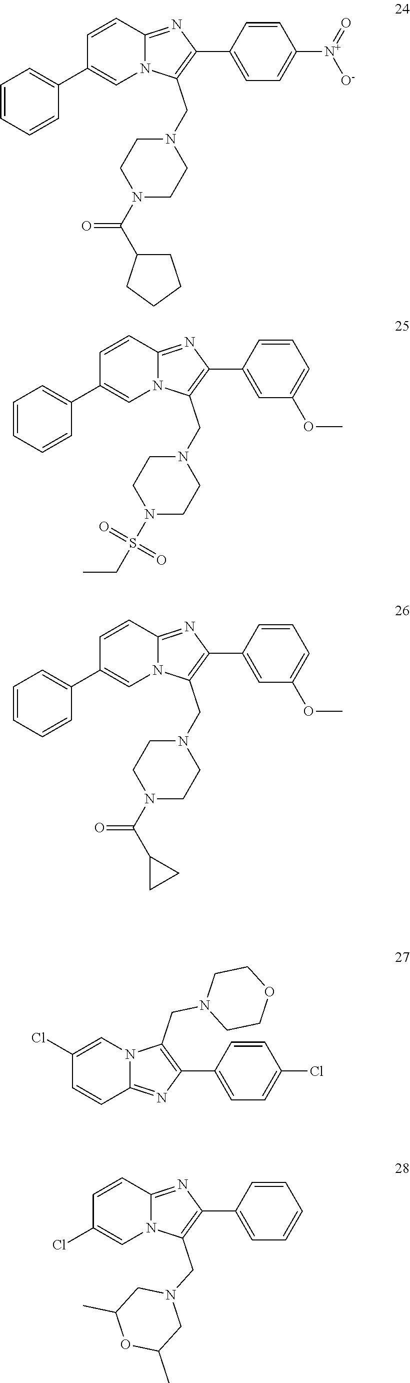

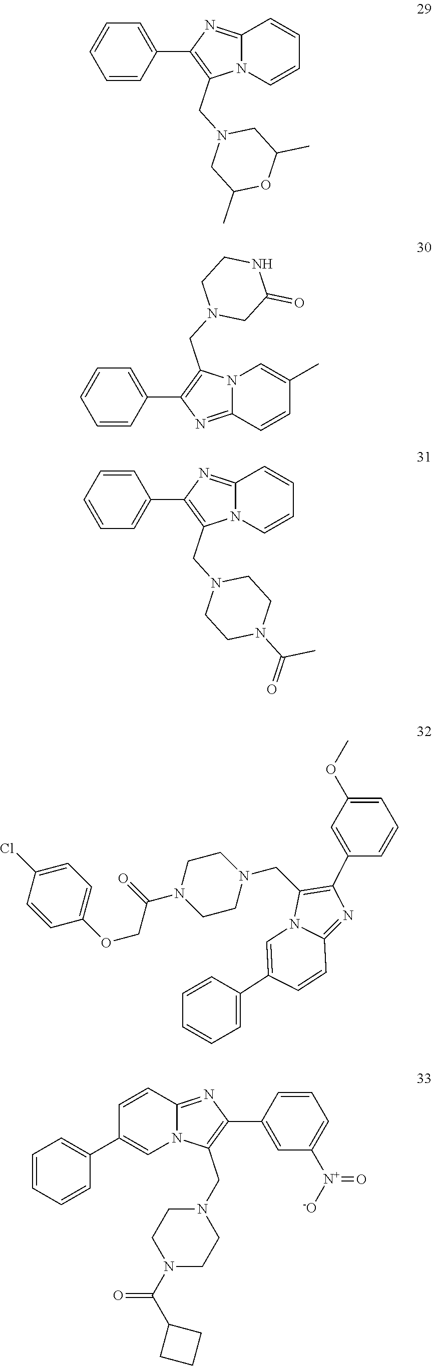

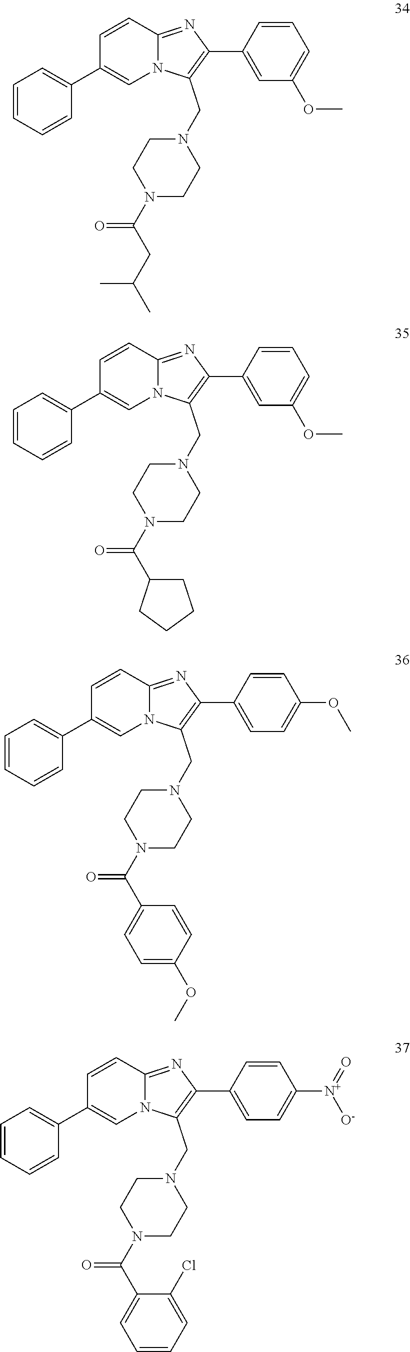

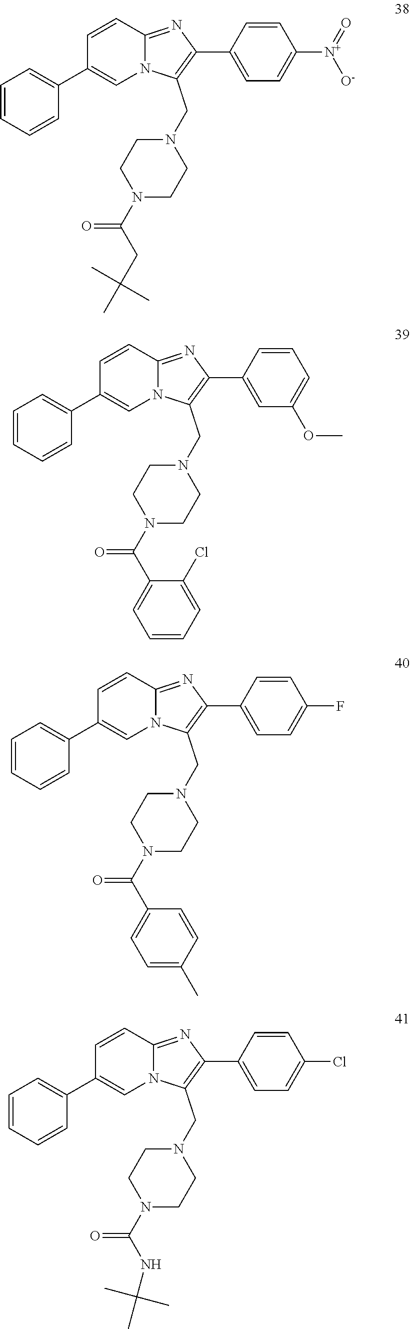

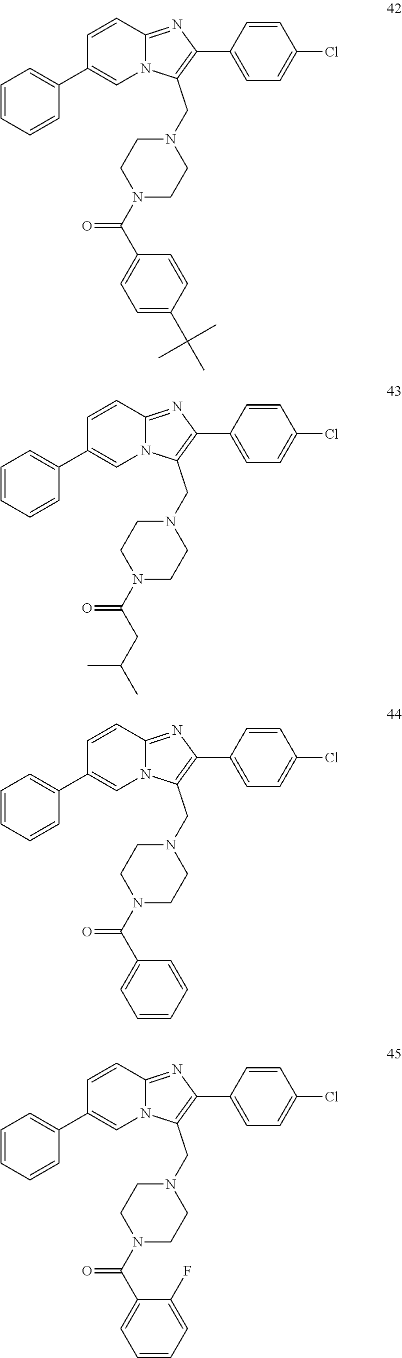

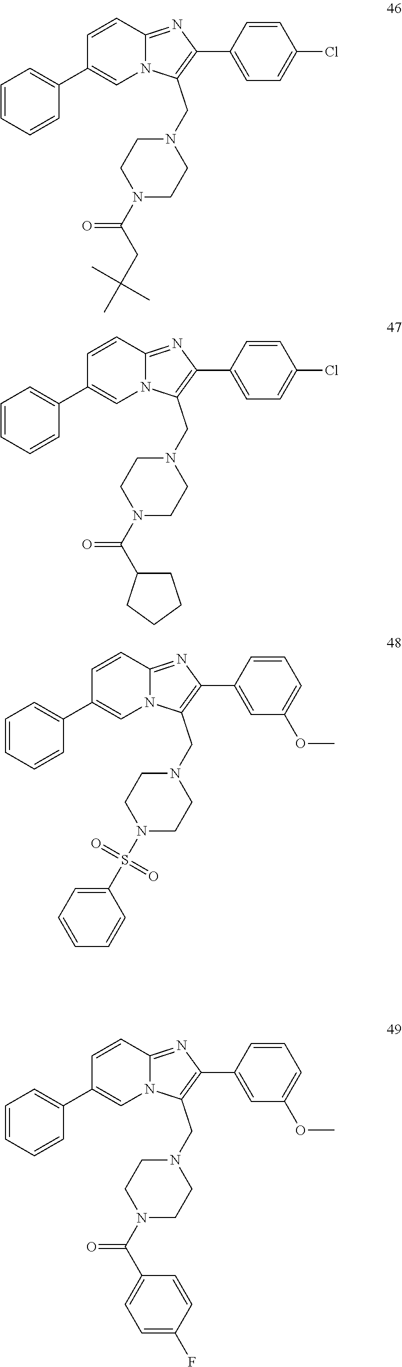

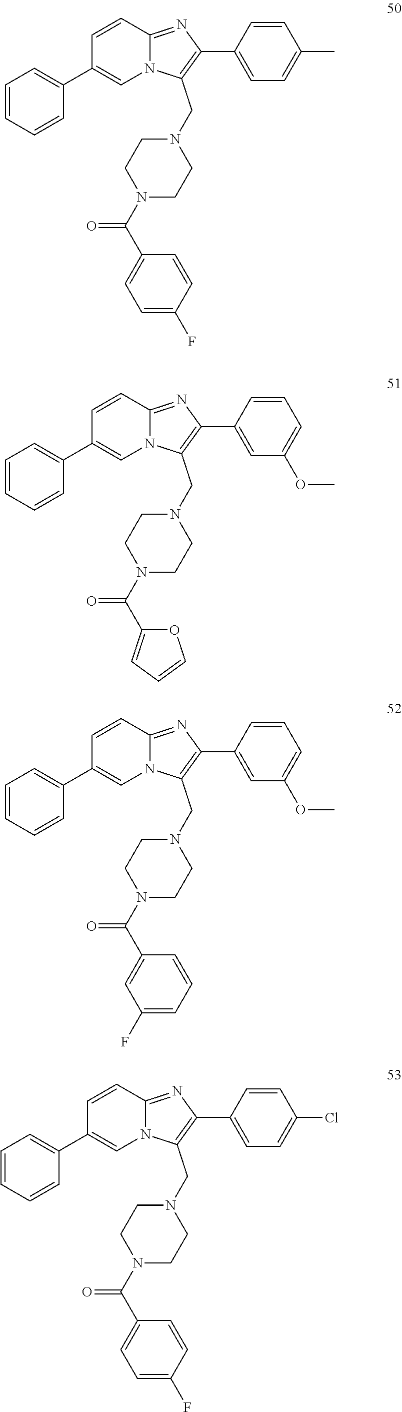

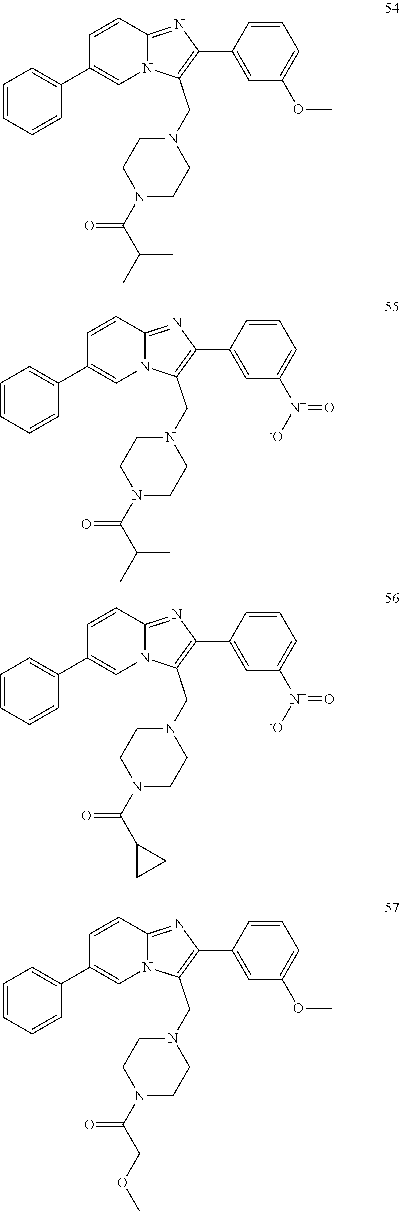

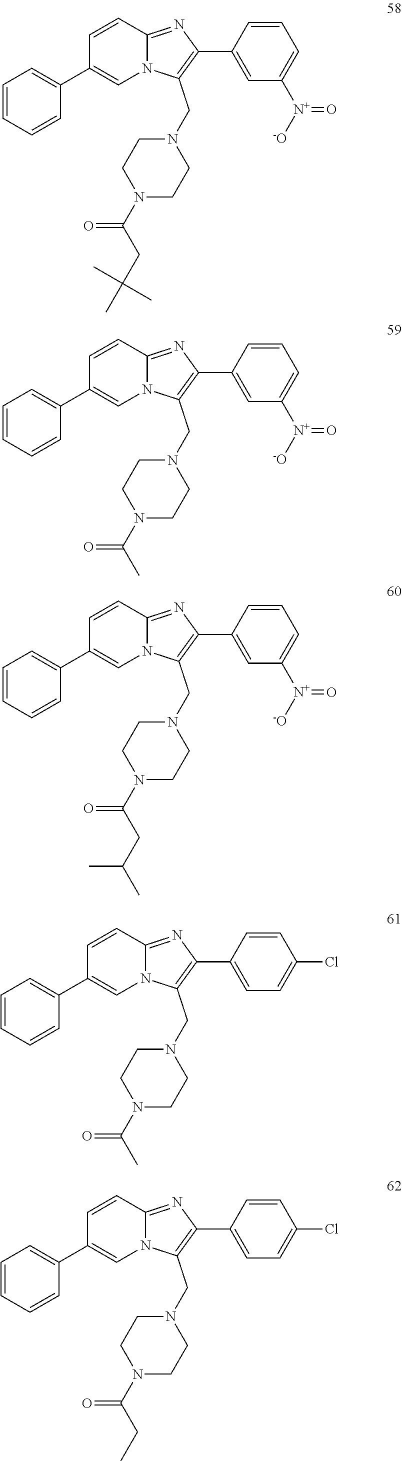

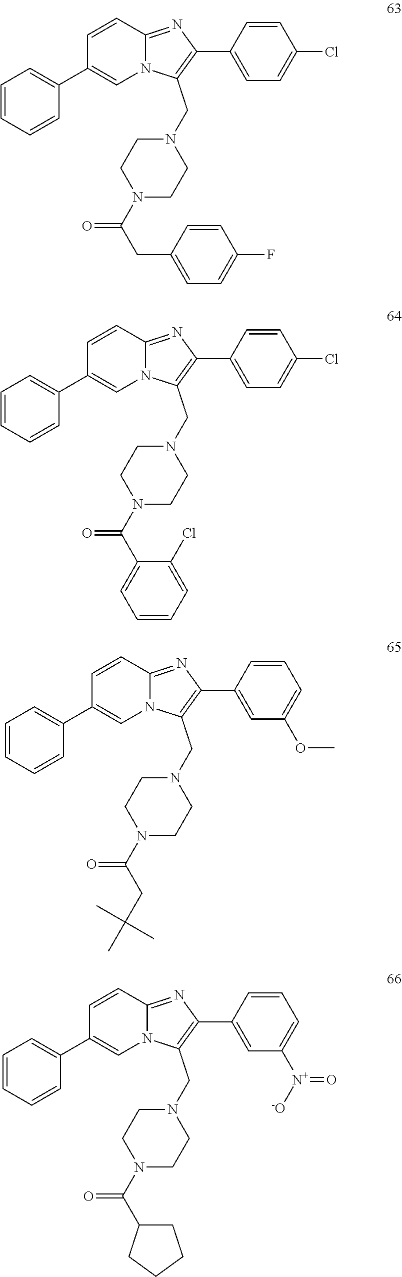

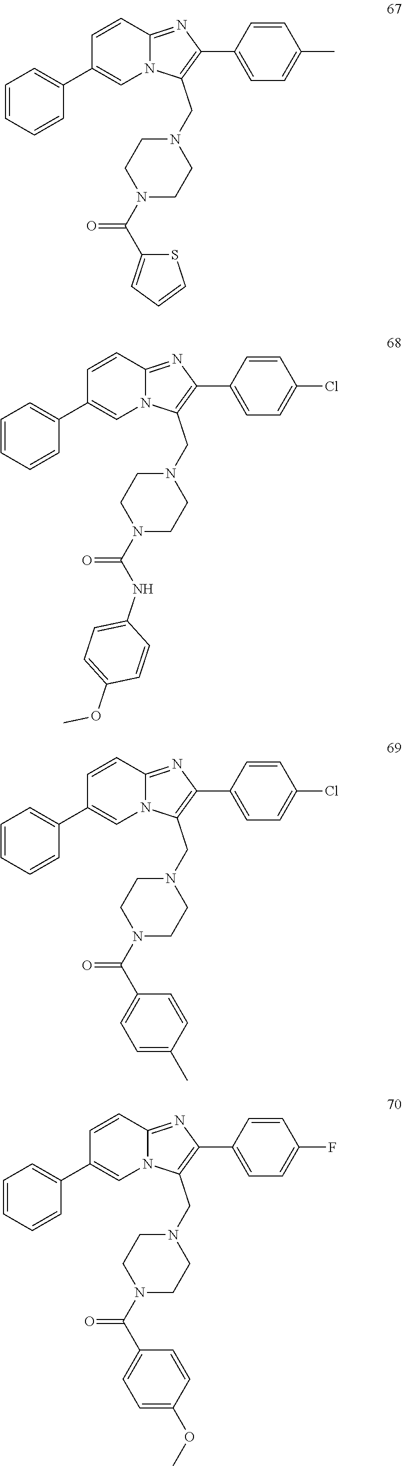

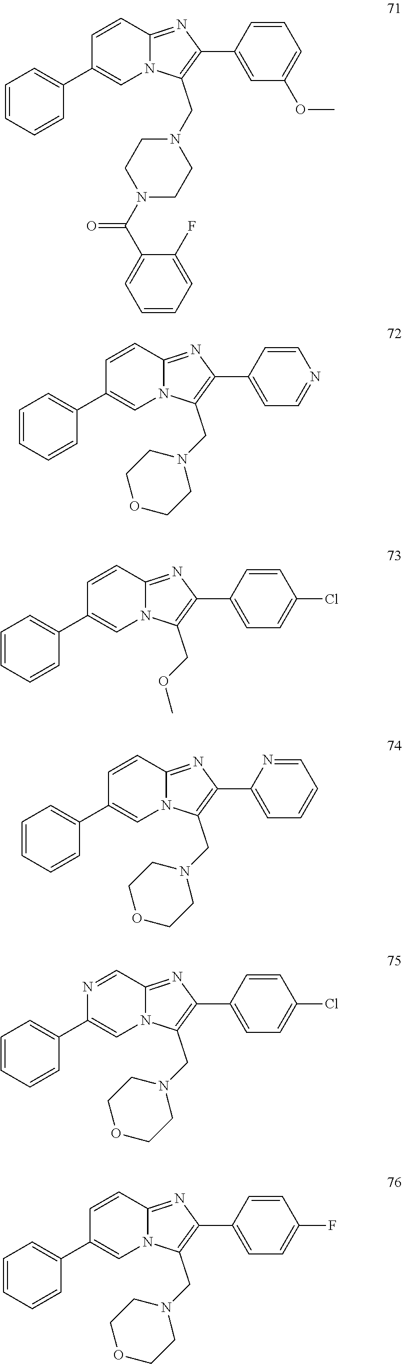

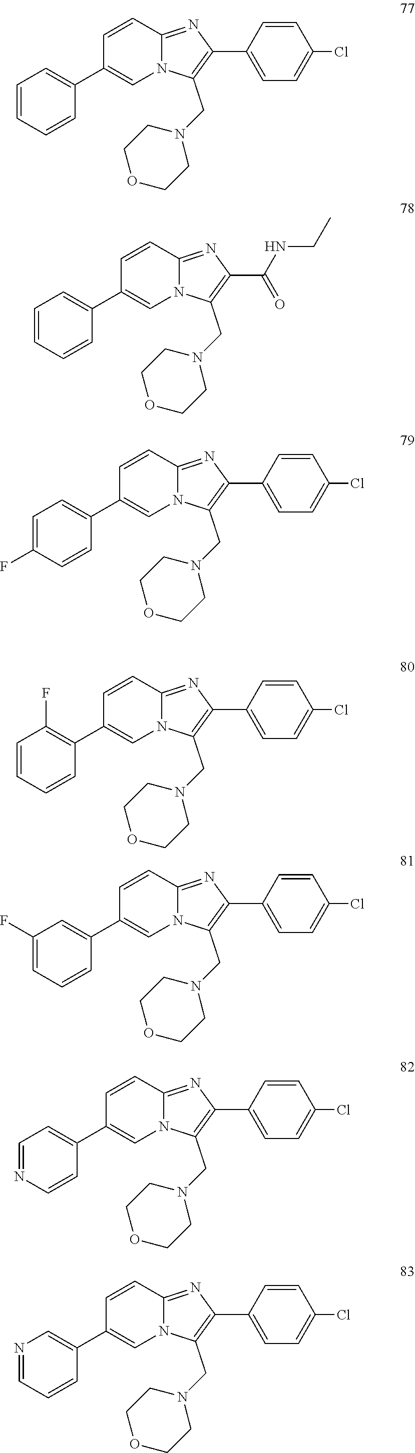

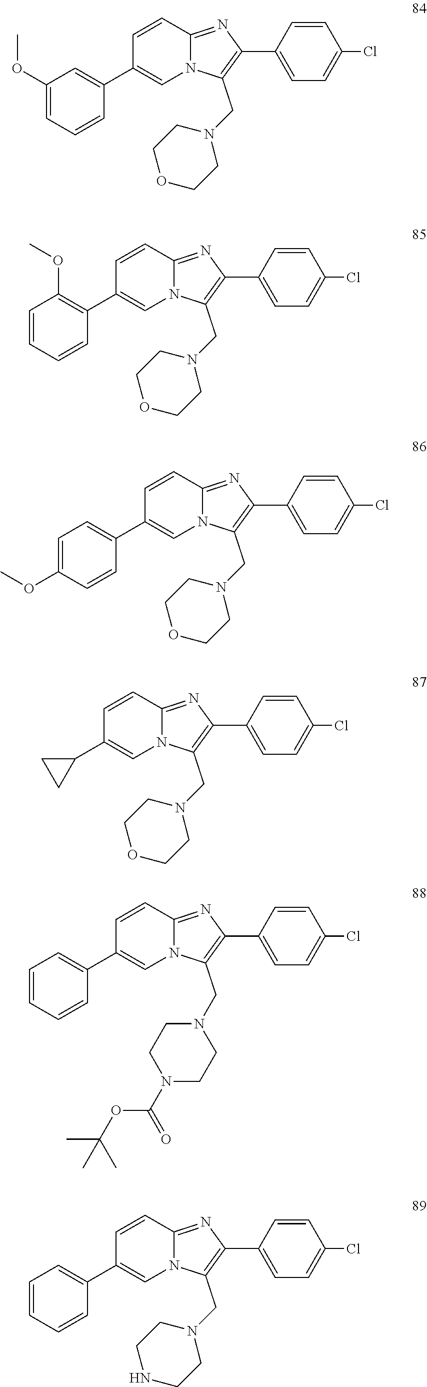

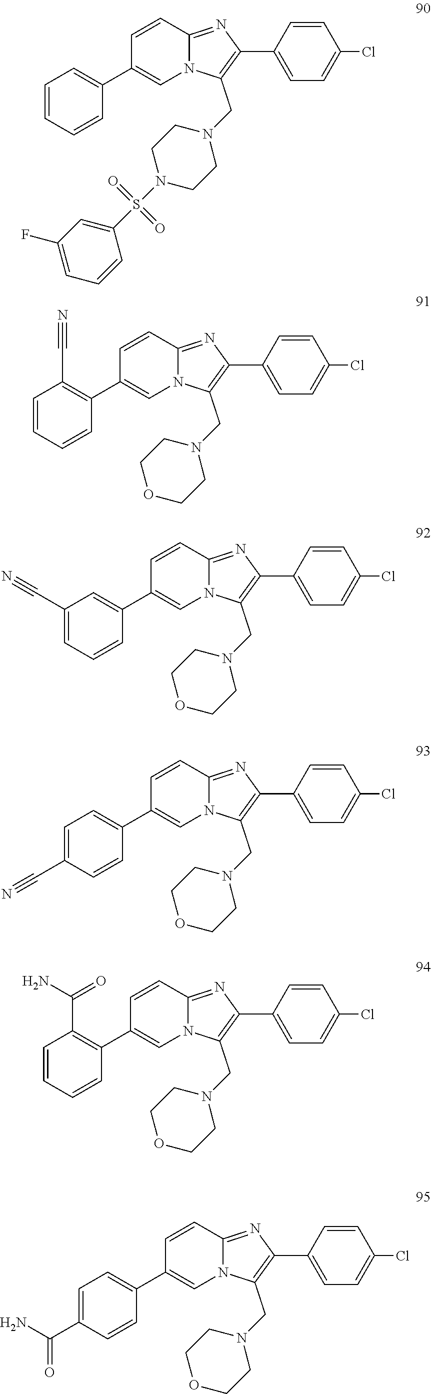

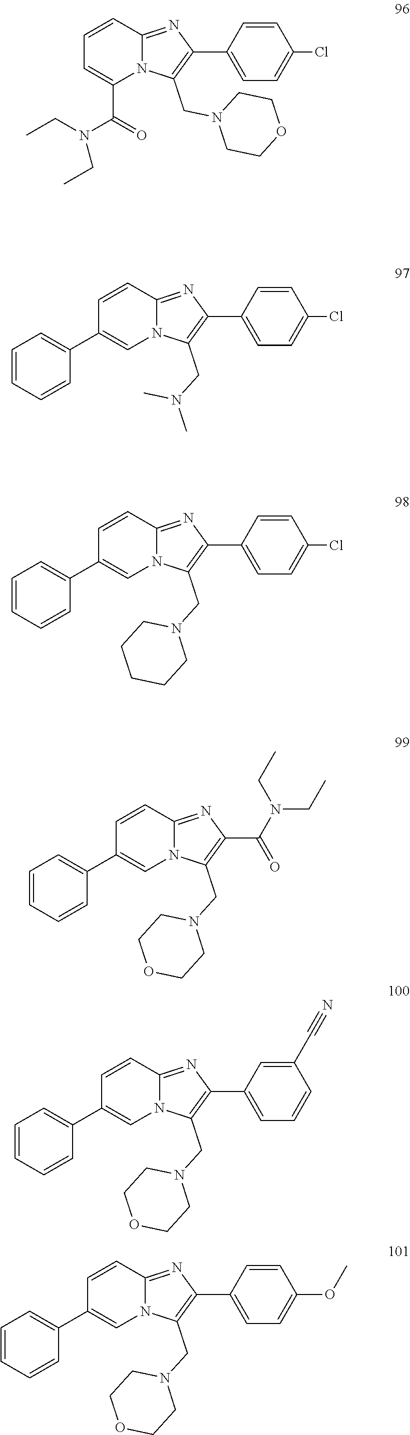

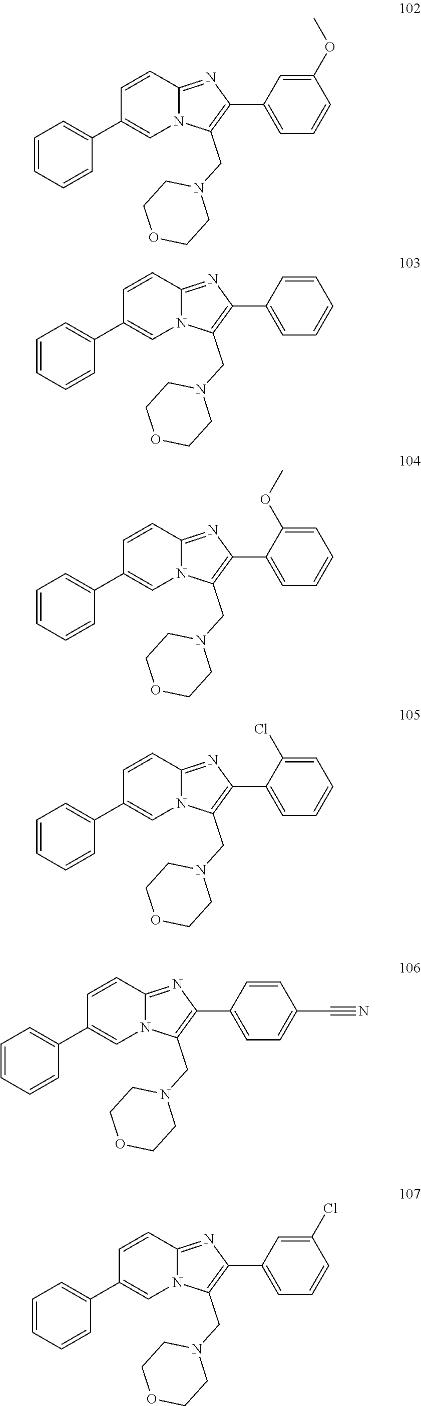

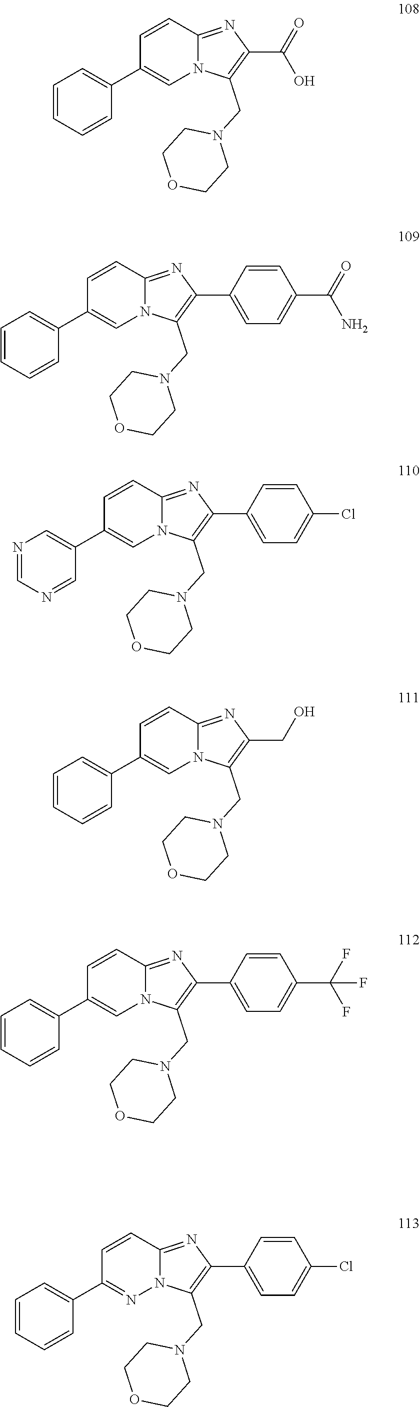 [SMILES: O=C(O)c1nc2ccc(-c3ccccc3)cn2c1CN1CCOCC1 ; NC(=O)c1ccc(-c2nc3ccc(-c4ccccc4)cn3c2CN2CCOCC2)cc1 ; Clc1ccc(-c2nc3ccc(-c4cncnc4)cn3c2CN2CCOCC2)cc1 ; OCc1nc2ccc(-c3ccccc3)cn2c1CN1CCOCC1 ; FC(F)(F)c1ccc(-c2nc3ccc(-c4ccccc4)cn3c2CN2CCOCC2)cc1 ; Clc1ccc(-c2nc3ccc(-c4ccccc4)nn3c2CN2CCOCC2)cc1]

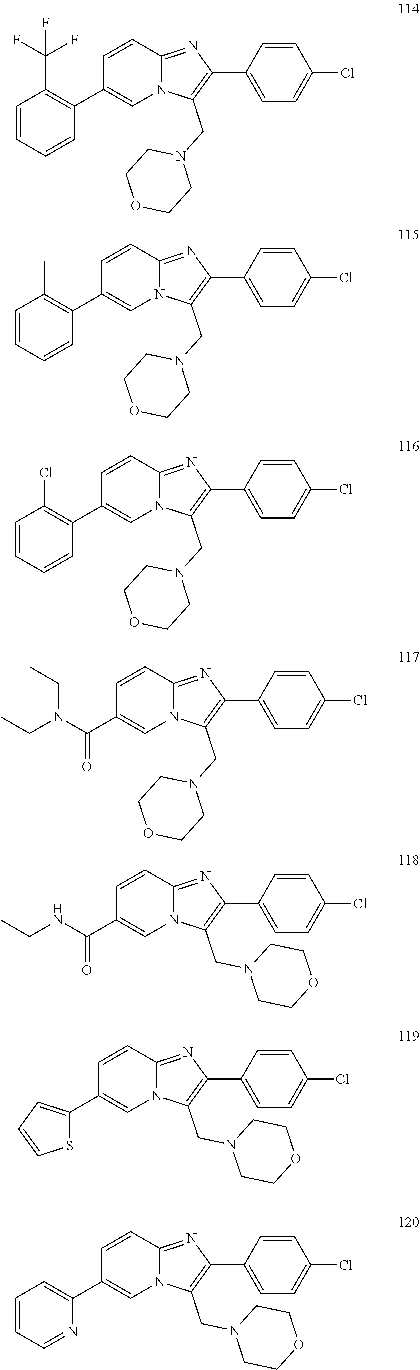

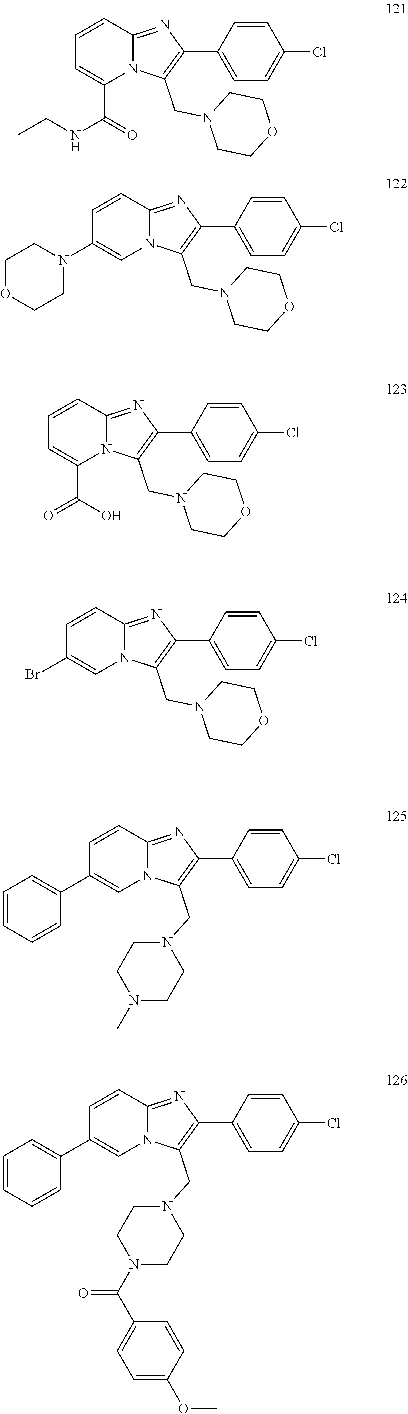

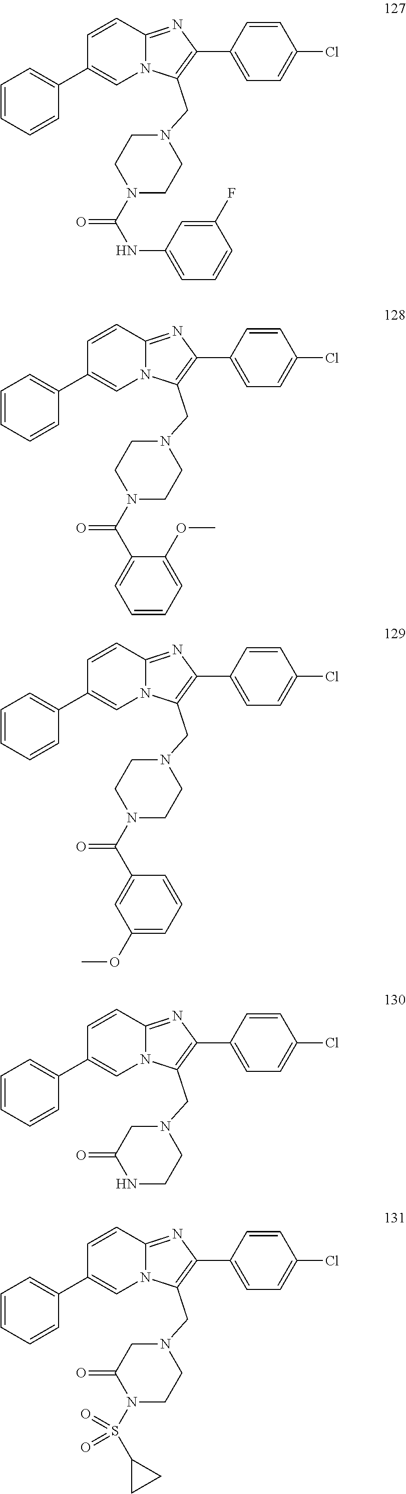

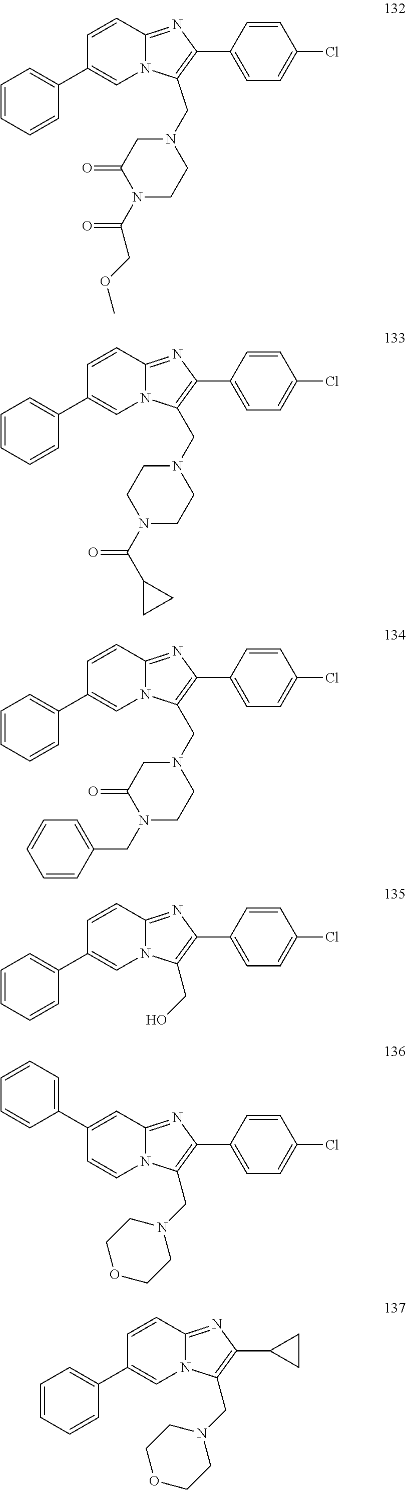

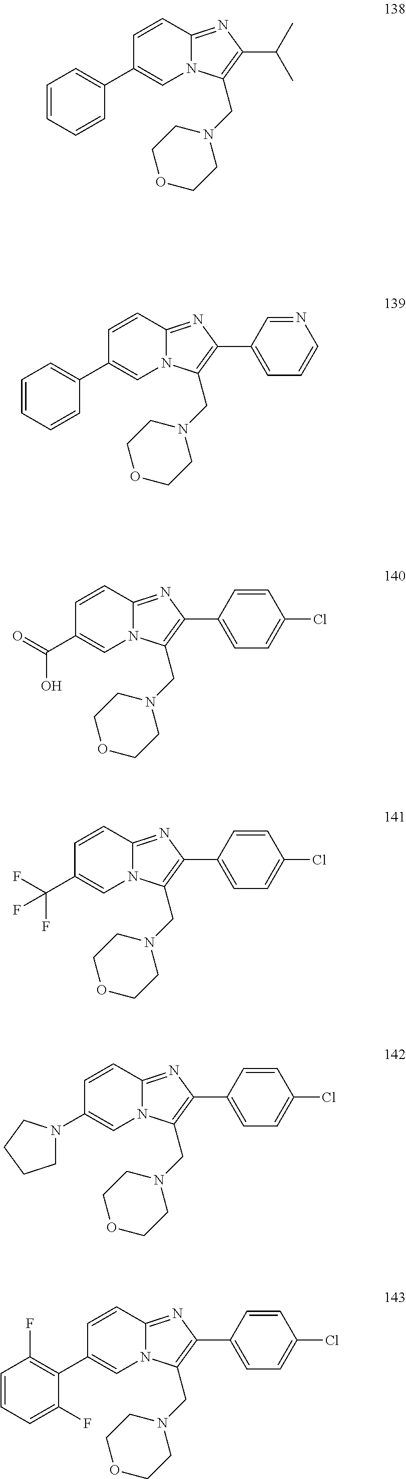 [SMILES: CC(C)c1nc2ccc(-c3ccccc3)cn2c1CN1CCOCC1 ; c1ccc(-c2ccc3nc(-c4cccnc4)c(CN4CCOCC4)n3c2)cc1 ; O=C(O)c1ccc2nc(-c3ccc(Cl)cc3)c(CN3CCOCC3)n2c1 ; FC(F)(F)c1ccc2nc(-c3ccc(Cl)cc3)c(CN3CCOCC3)n2c1 ; Clc1ccc(-c2nc3ccc(N4CCCC4)cn3c2CN2CCOCC2)cc1 ; Fc1cccc(F)c1-c1ccc2nc(-c3ccc(Cl)cc3)c(CN3CCOCC3)n2c1]

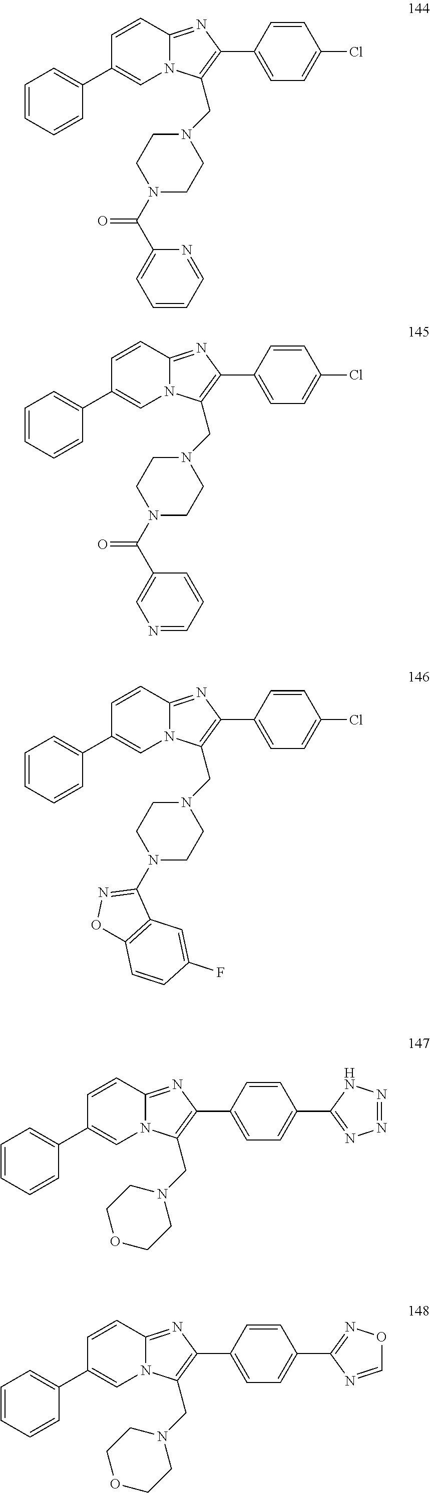

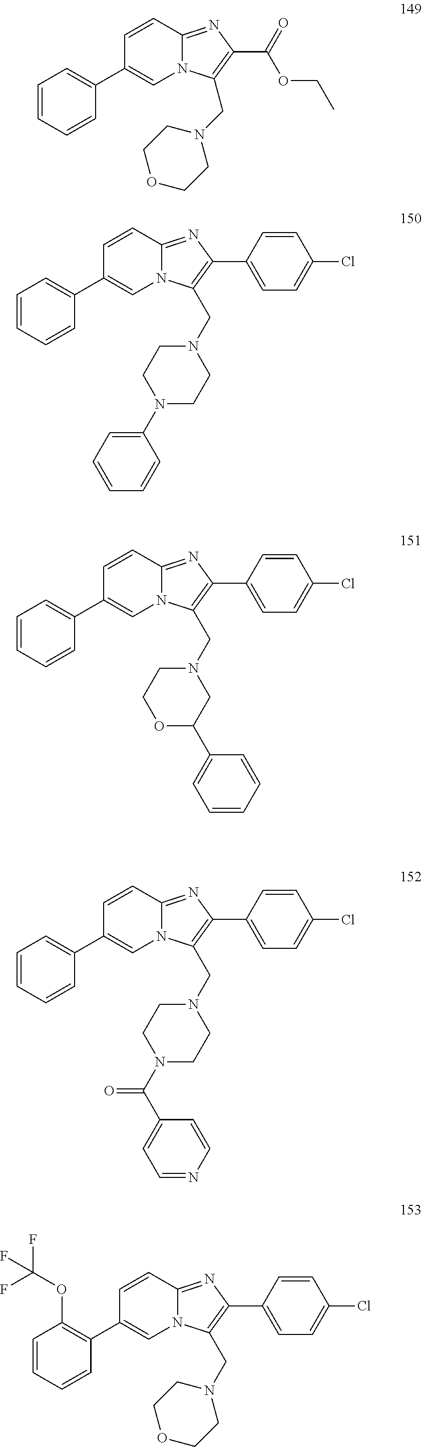

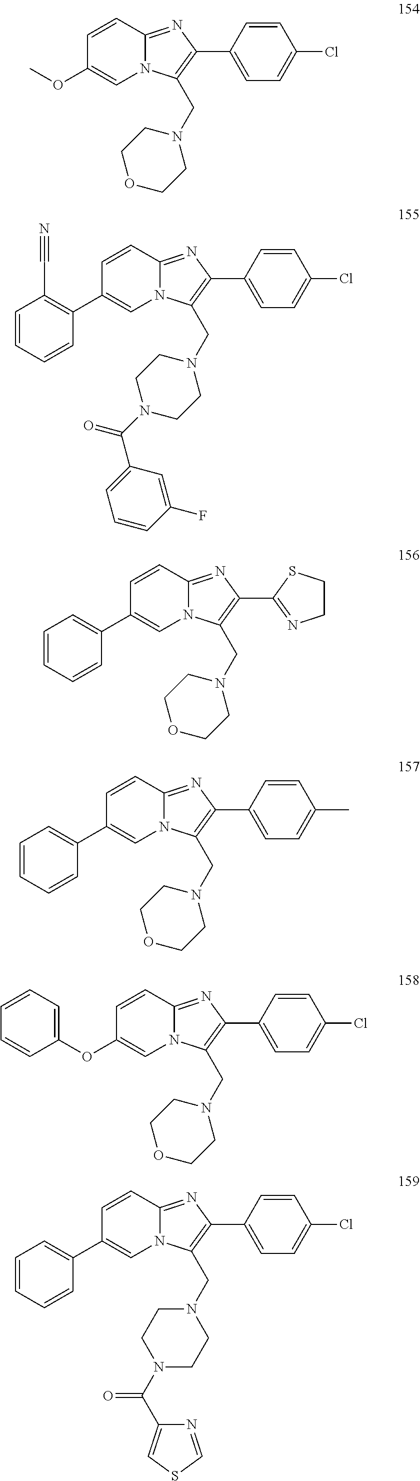

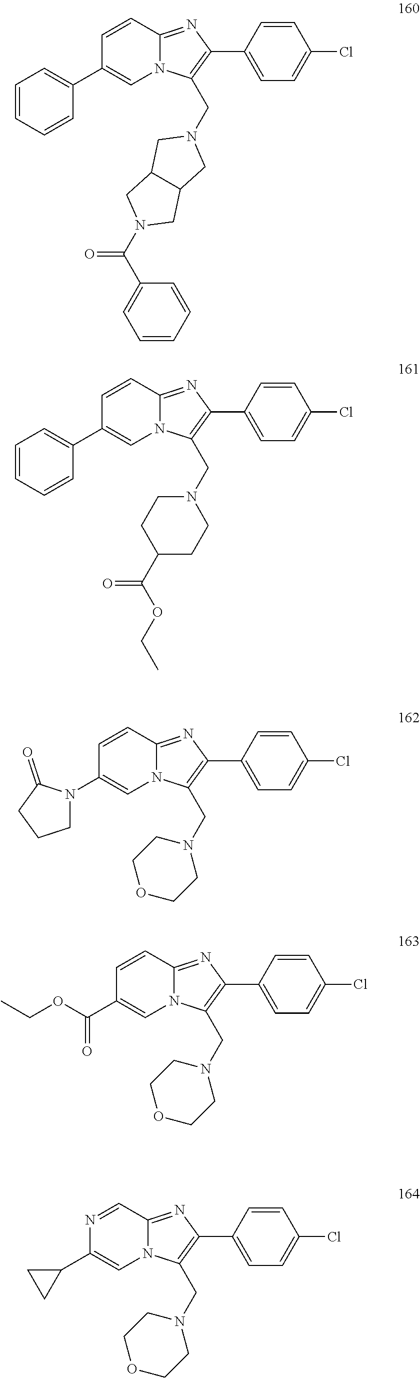

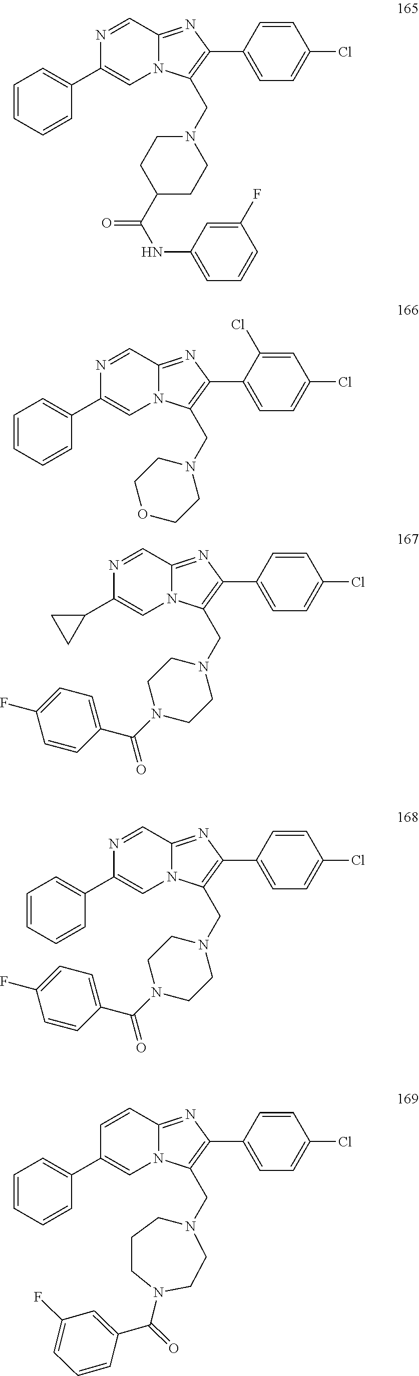

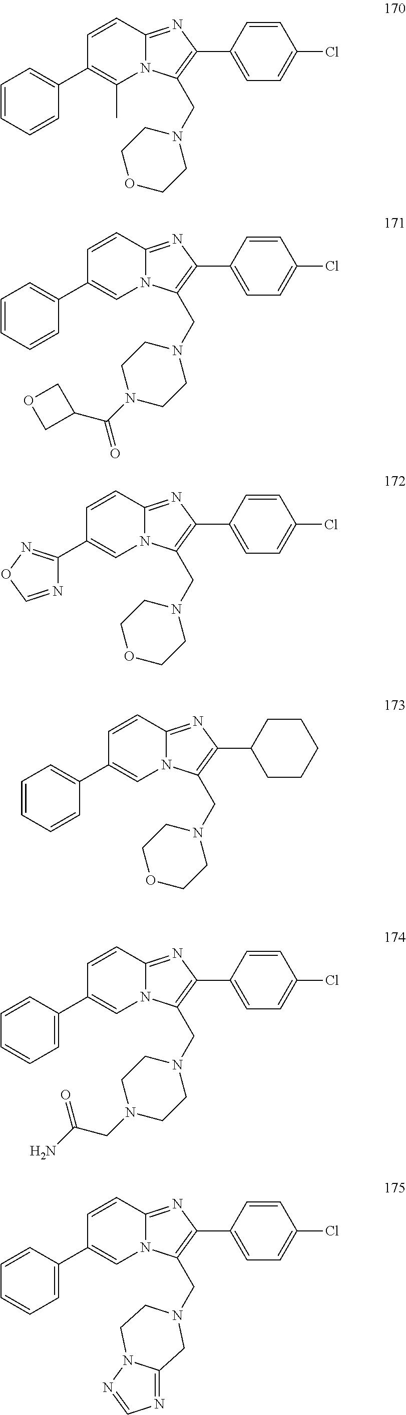

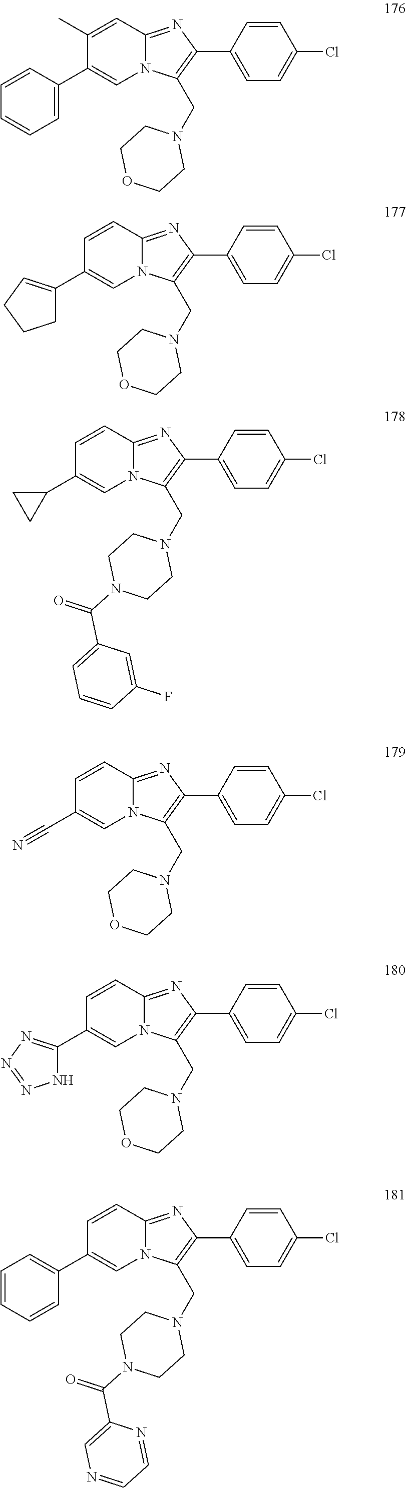

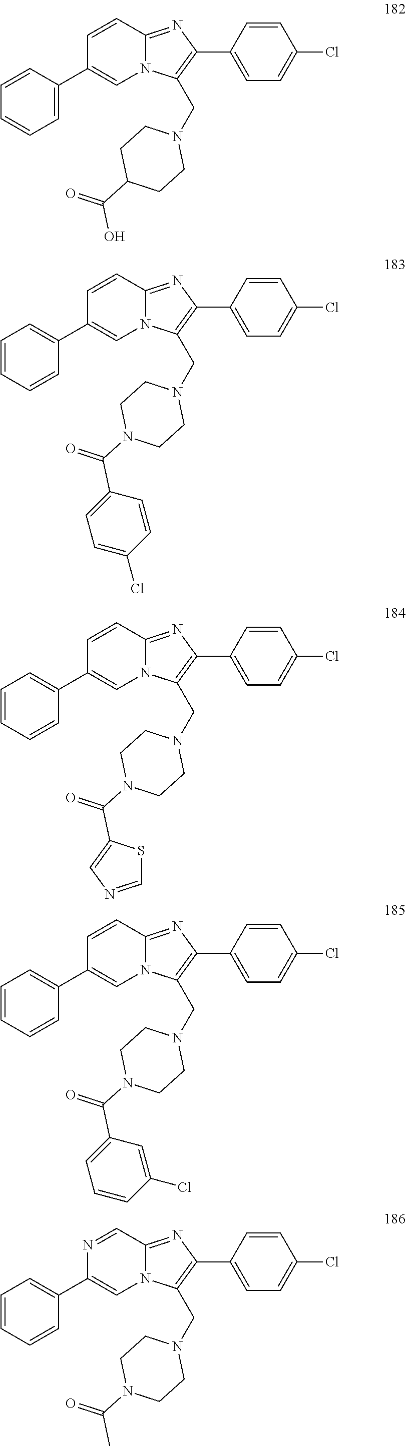

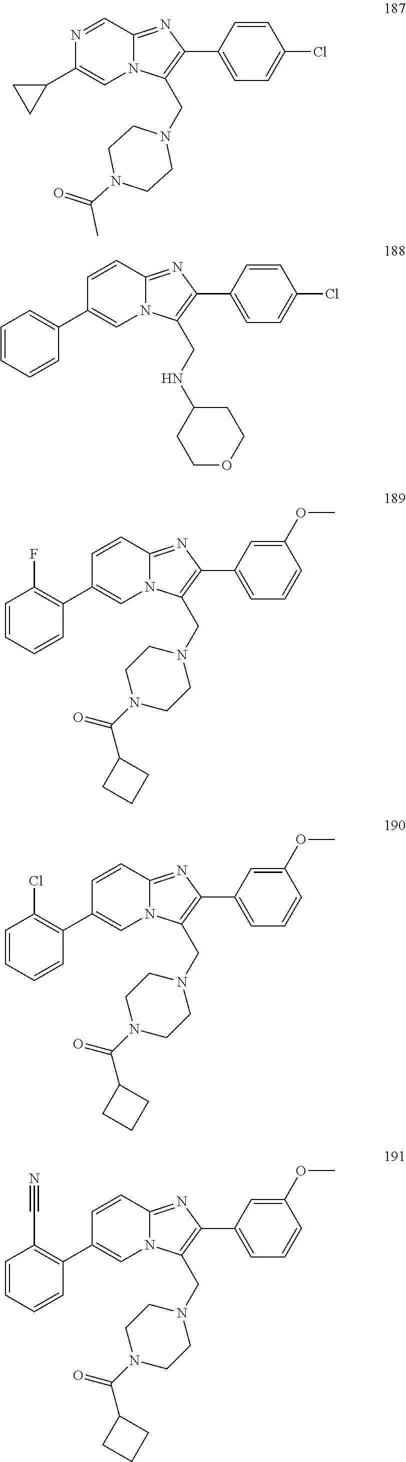

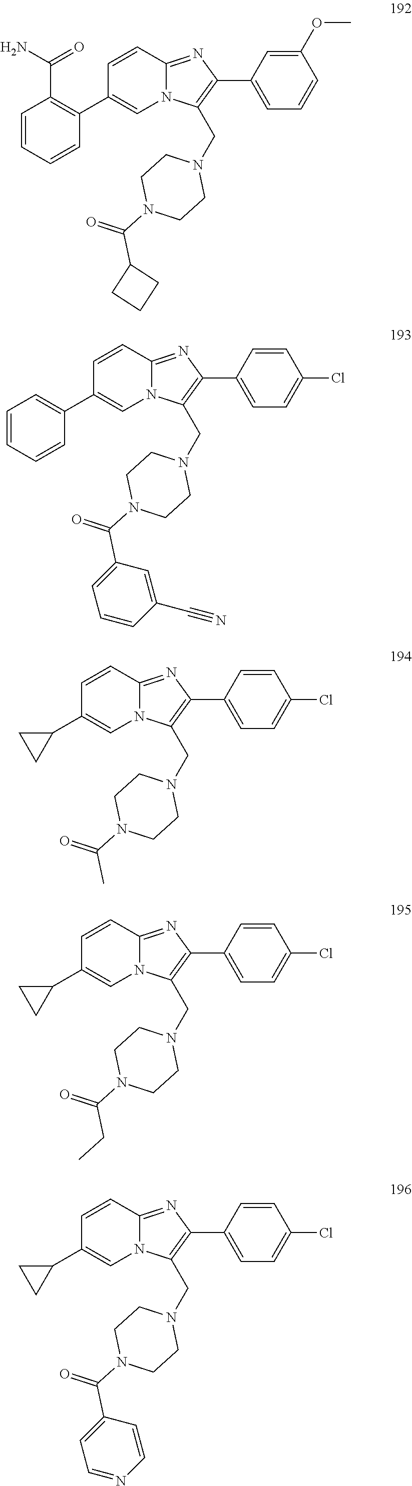

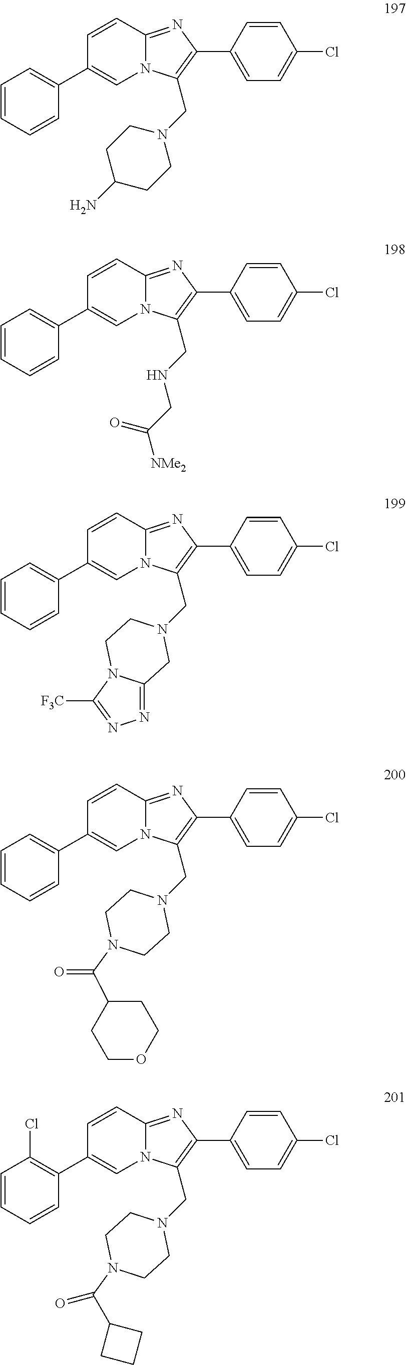 [SMILES: NC1CCN(Cc2c(-c3ccc(Cl)cc3)nc3ccc(-c4ccccc4)cn23)CC1 ; CN(C)C(=O)CNCc1c(-c2ccc(Cl)cc2)nc2ccc(-c3ccccc3)cn12 ; FC(F)(F)c1nnc2n1CCN(Cc1c(-c3ccc(Cl)cc3)nc3ccc(-c4ccccc4)cn13)C2 ; O=C(C1CCOCC1)N1CCN(Cc2c(-c3ccc(Cl)cc3)nc3ccc(-c4ccccc4)cn23)CC1 ; O=C(C1CCC1)N1CCN(Cc2c(-c3ccc(Cl)cc3)nc3ccc(-c4ccccc4Cl)cn23)CC1]

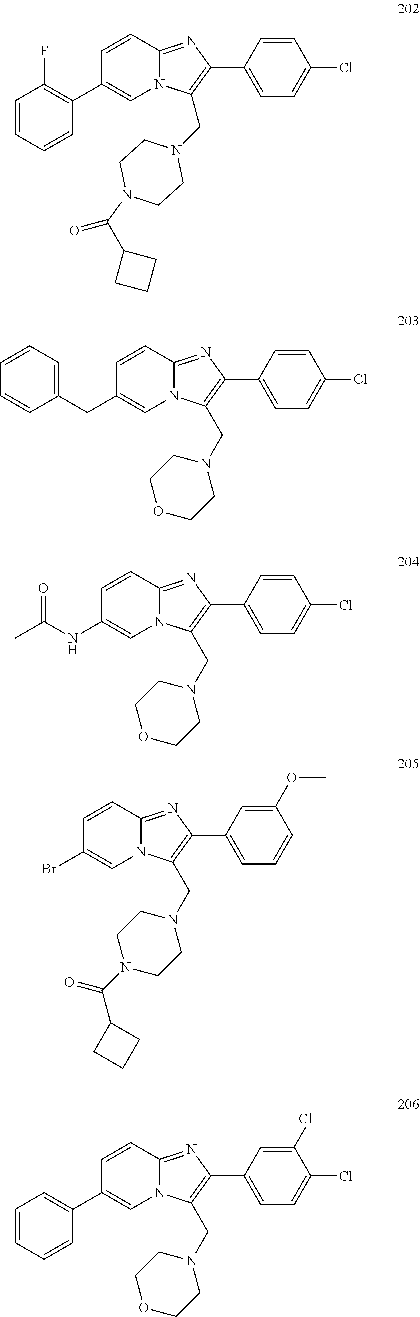 [SMILES: O=C(C1CCC1)N1CCN(Cc2c(-c3ccc(Cl)cc3)nc3ccc(-c4ccccc4F)cn23)CC1 ; Clc1ccc(-c2nc3ccc(Cc4ccccc4)cn3c2CN2CCOCC2)cc1 ; CC(=O)Nc1ccc2nc(-c3ccc(Cl)cc3)c(CN3CCOCC3)n2c1 ; COc1cccc(-c2nc3ccc(Br)cn3c2CN2CCN(C(=O)C3CCC3)CC2)c1 ; Clc1ccc(-c2nc3ccc(-c4ccccc4)cn3c2CN2CCOCC2)cc1Cl]

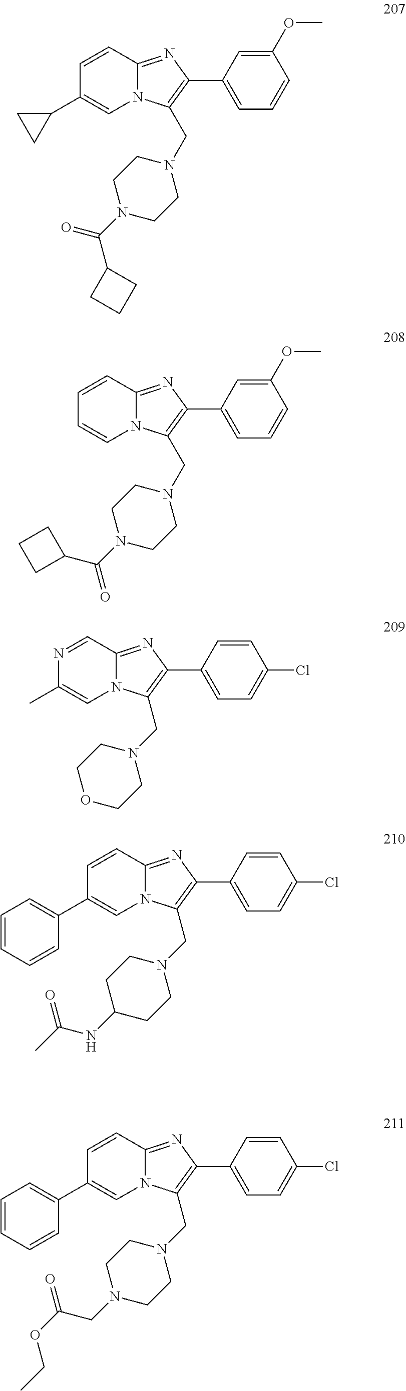

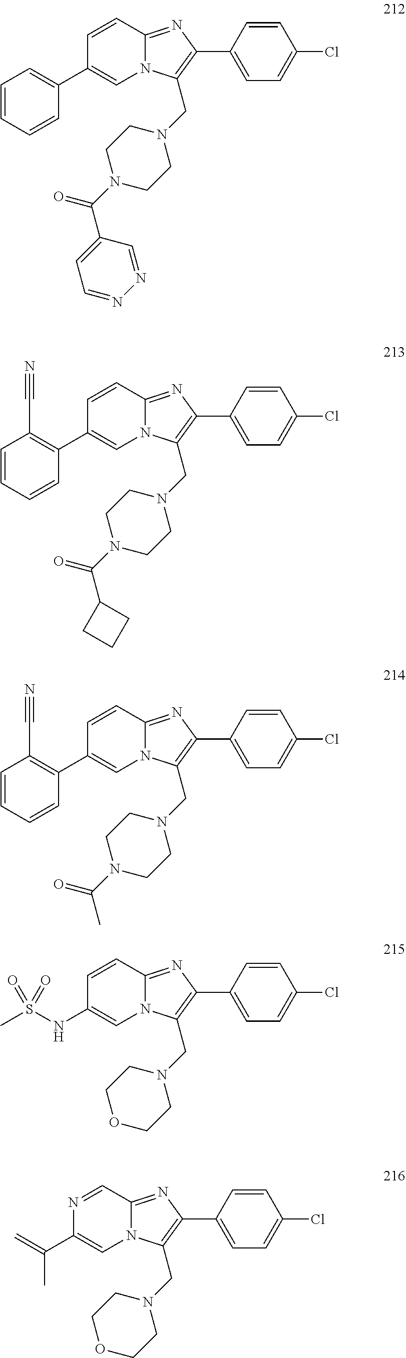

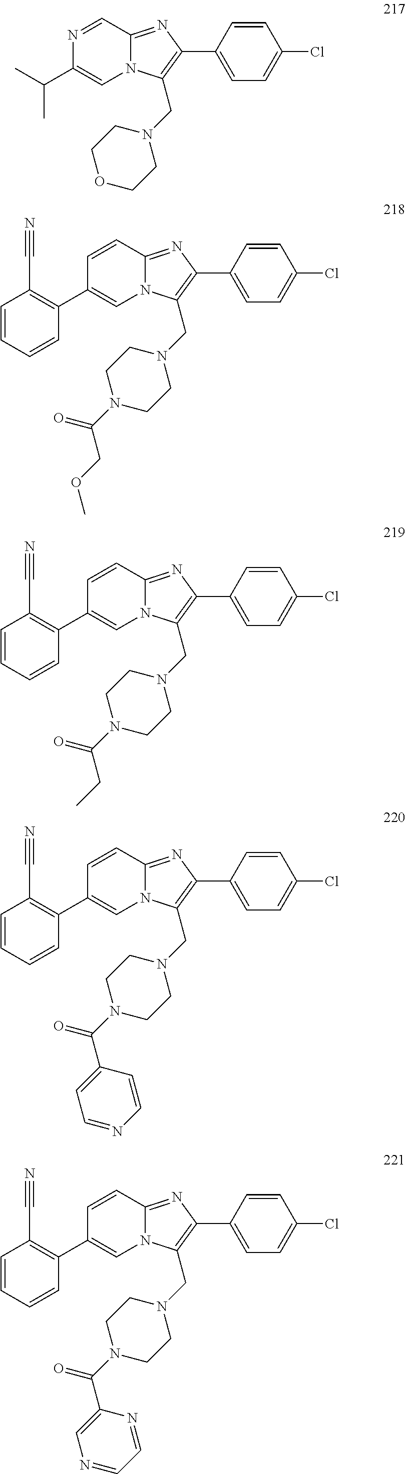 [SMILES: CC(C)c1cn2c(CN3CCOCC3)c(-c3ccc(Cl)cc3)nc2cn1 ; COCC(=O)N1CCN(Cc2c(-c3ccc(Cl)cc3)nc3ccc(-c4ccccc4C#N)cn23)CC1 ; CCC(=O)N1CCN(Cc2c(-c3ccc(Cl)cc3)nc3ccc(-c4ccccc4C#N)cn23)CC1 ; N#Cc1ccccc1-c1ccc2nc(-c3ccc(Cl)cc3)c(CN3CCN(C(=O)c4ccncc4)CC3)n2c1 ; N#Cc1ccccc1-c1ccc2nc(-c3ccc(Cl)cc3)c(CN3CCN(C(=O)c4cnccn4)CC3)n2c1]

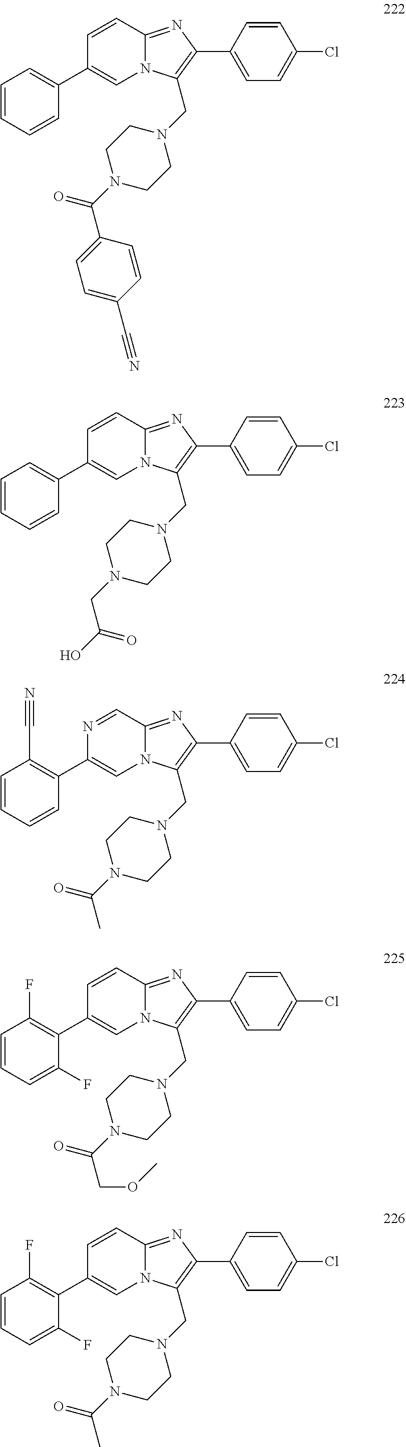

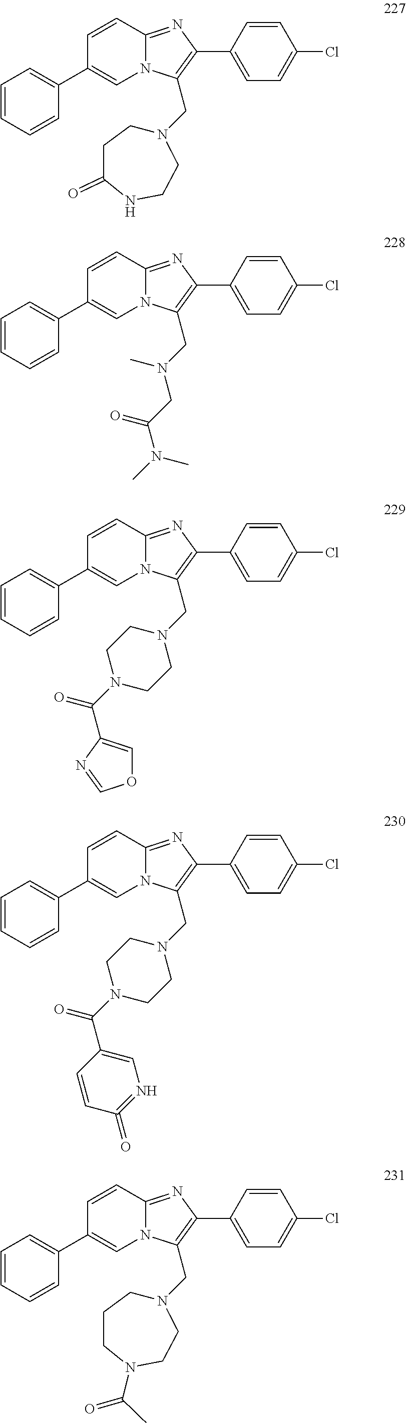

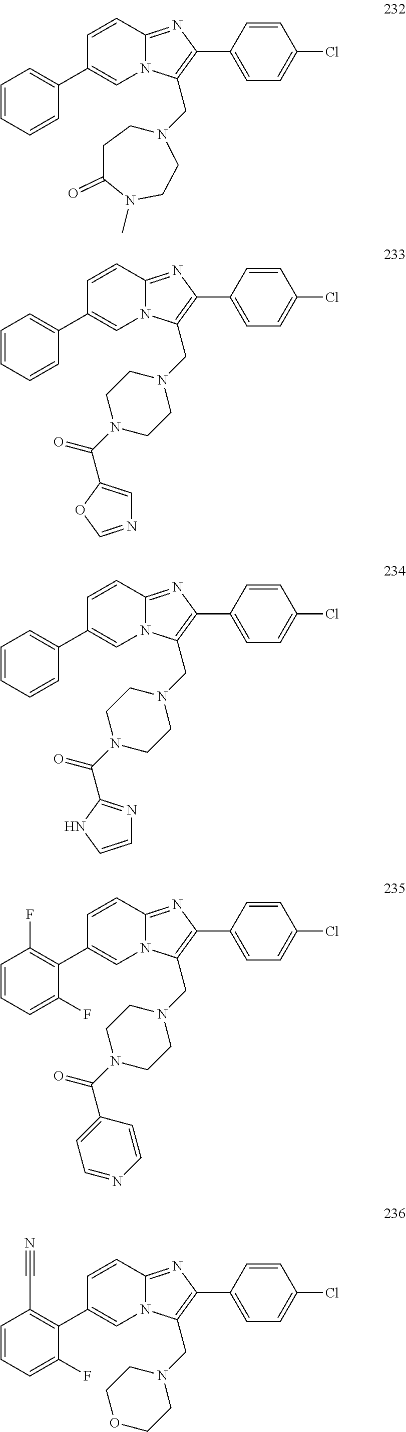

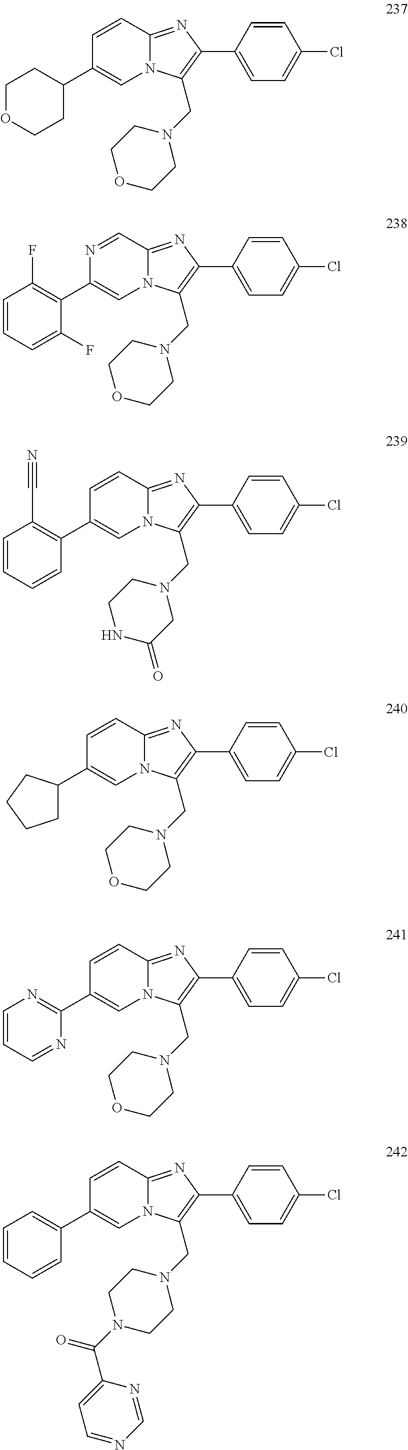 [SMILES: Clc1ccc(-c2nc3ccc(C4CCOCC4)cn3c2CN2CCOCC2)cc1 ; Fc1cccc(F)c1-c1cn2c(CN3CCOCC3)c(-c3ccc(Cl)cc3)nc2cn1 ; N#Cc1ccccc1-c1ccc2nc(-c3ccc(Cl)cc3)c(CN3CCNC(=O)C3)n2c1 ; Clc1ccc(-c2nc3ccc(C4CCCC4)cn3c2CN2CCOCC2)cc1 ; Clc1ccc(-c2nc3ccc(-c4ncccn4)cn3c2CN2CCOCC2)cc1 ; O=C(c1ccncn1)N1CCN(Cc2c(-c3ccc(Cl)cc3)nc3ccc(-c4ccccc4)cn23)CC1]

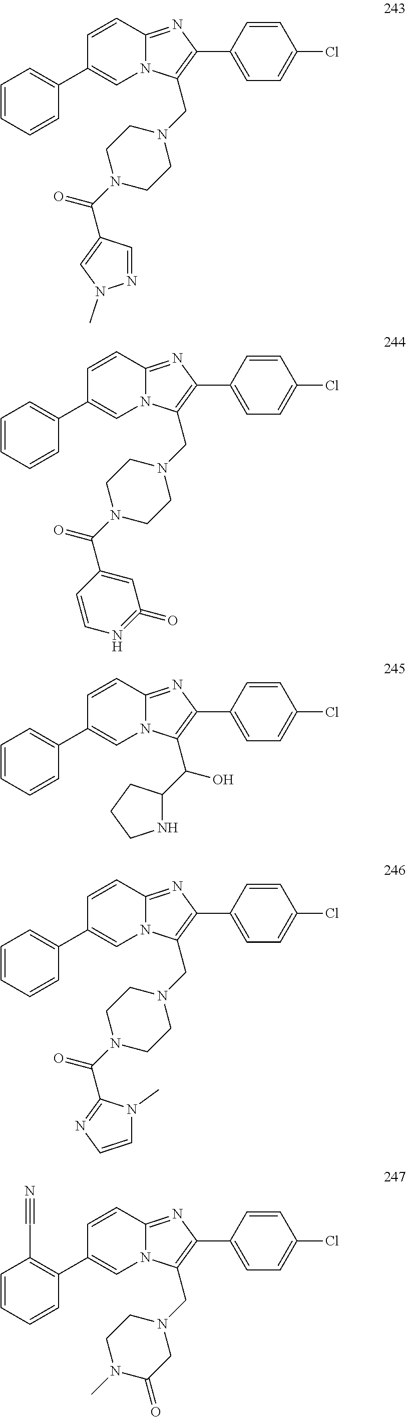

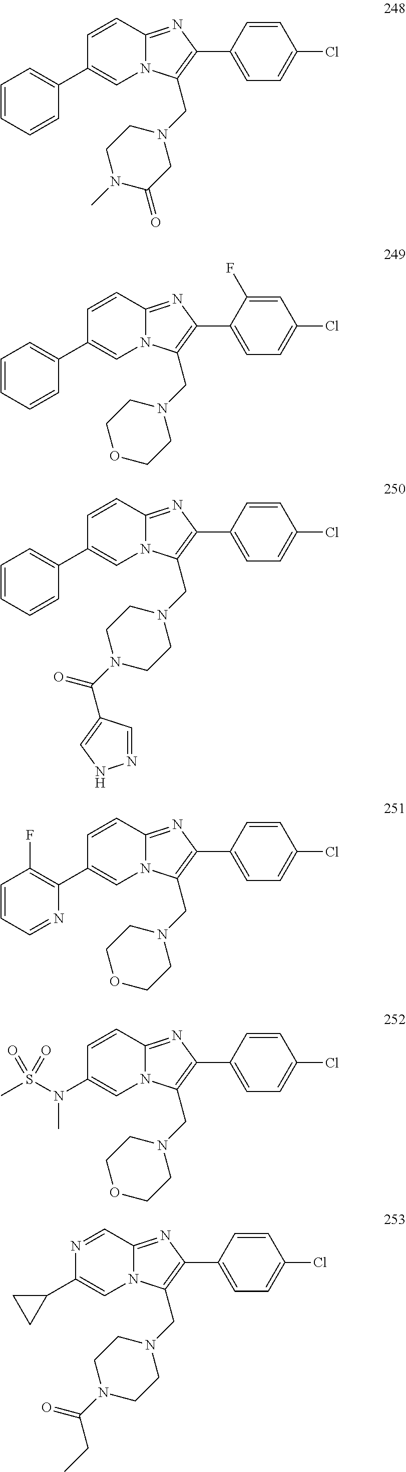

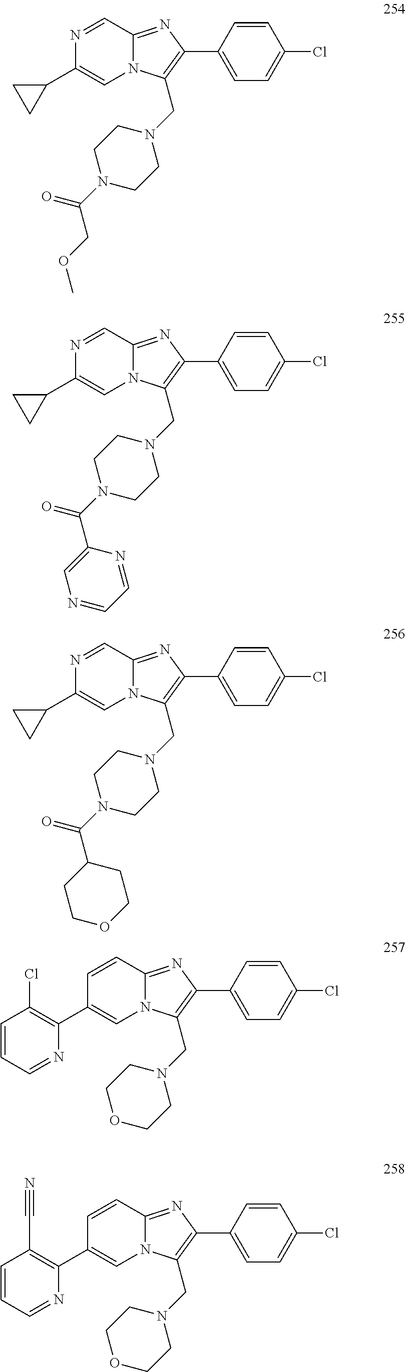

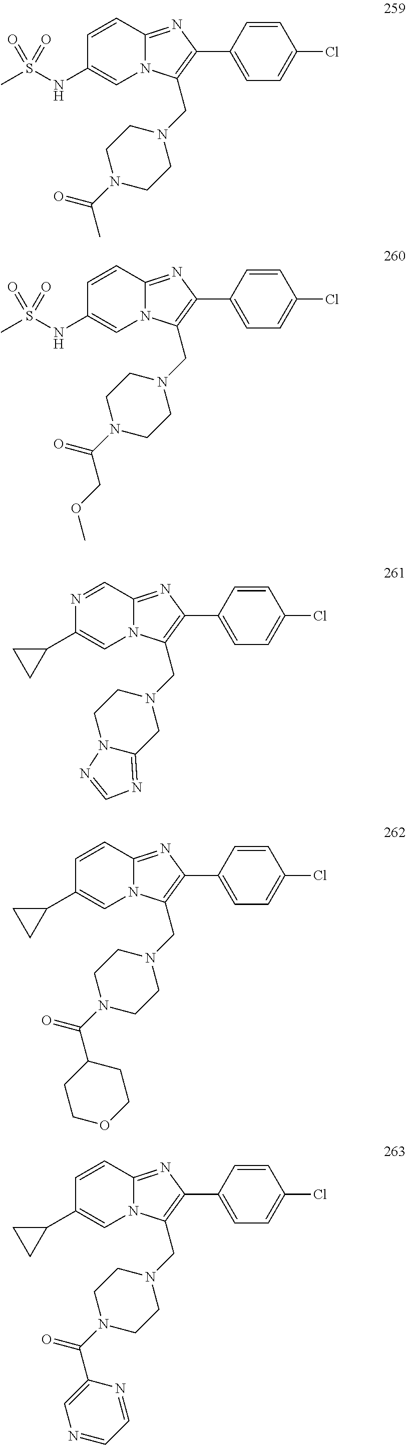

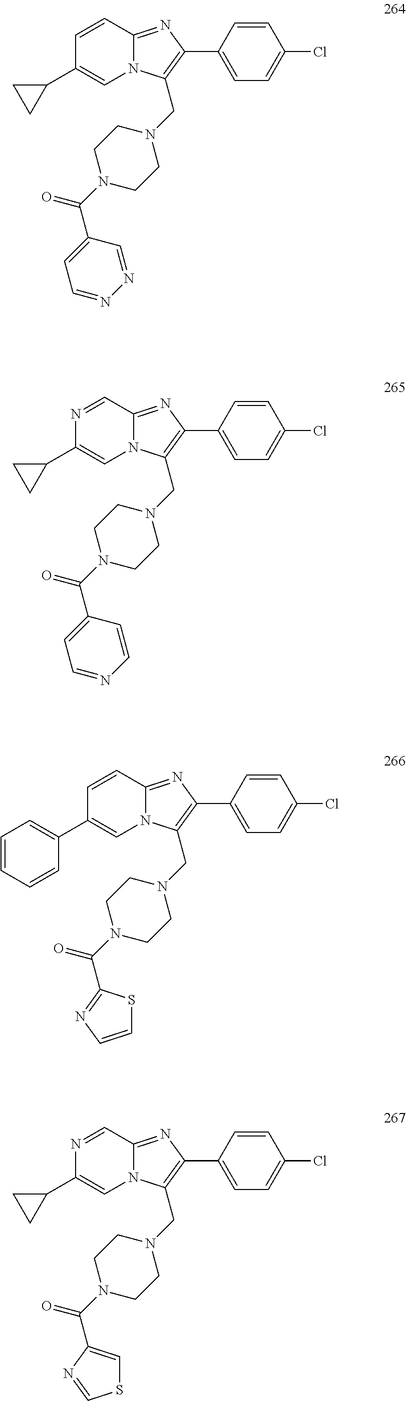 [SMILES: O=C(c1ccnnc1)N1CCN(Cc2c(-c3ccc(Cl)cc3)nc3ccc(C4CC4)cn23)CC1 ; O=C(c1ccncc1)N1CCN(Cc2c(-c3ccc(Cl)cc3)nc3cnc(C4CC4)cn23)CC1 ; O=C(c1nccs1)N1CCN(Cc2c(-c3ccc(Cl)cc3)nc3ccc(-c4ccccc4)cn23)CC1 ; O=C(c1cscn1)N1CCN(Cc2c(-c3ccc(Cl)cc3)nc3cnc(C4CC4)cn23)CC1]

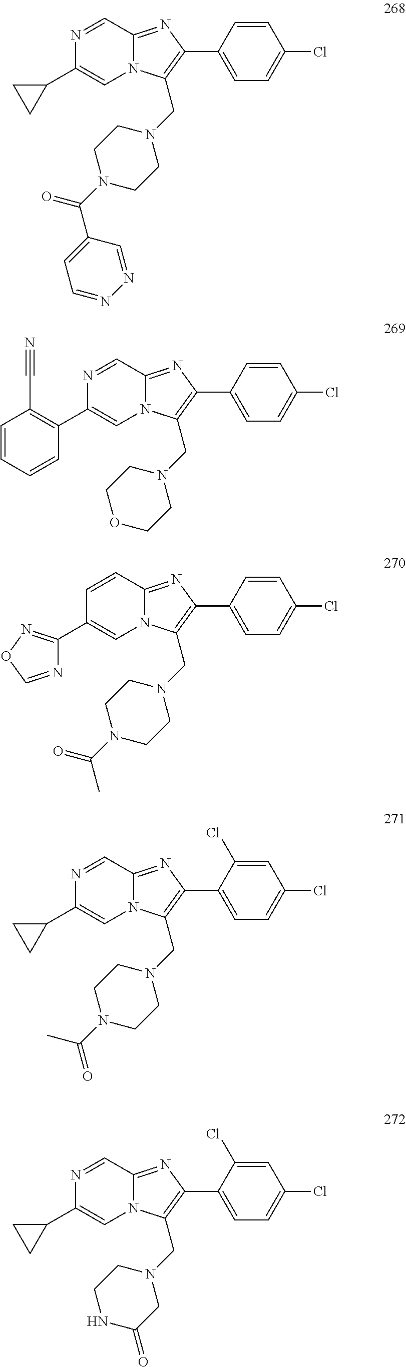 [SMILES: O=C(c1ccnnc1)N1CCN(Cc2c(-c3ccc(Cl)cc3)nc3cnc(C4CC4)cn23)CC1 ; N#Cc1ccccc1-c1cn2c(CN3CCOCC3)c(-c3ccc(Cl)cc3)nc2cn1 ; CC(=O)N1CCN(Cc2c(-c3ccc(Cl)cc3)nc3ccc(-c4ncon4)cn23)CC1 ; CC(=O)N1CCN(Cc2c(-c3ccc(Cl)cc3Cl)nc3cnc(C4CC4)cn23)CC1 ; O=C1CN(Cc2c(-c3ccc(Cl)cc3Cl)nc3cnc(C4CC4)cn23)CCN1]

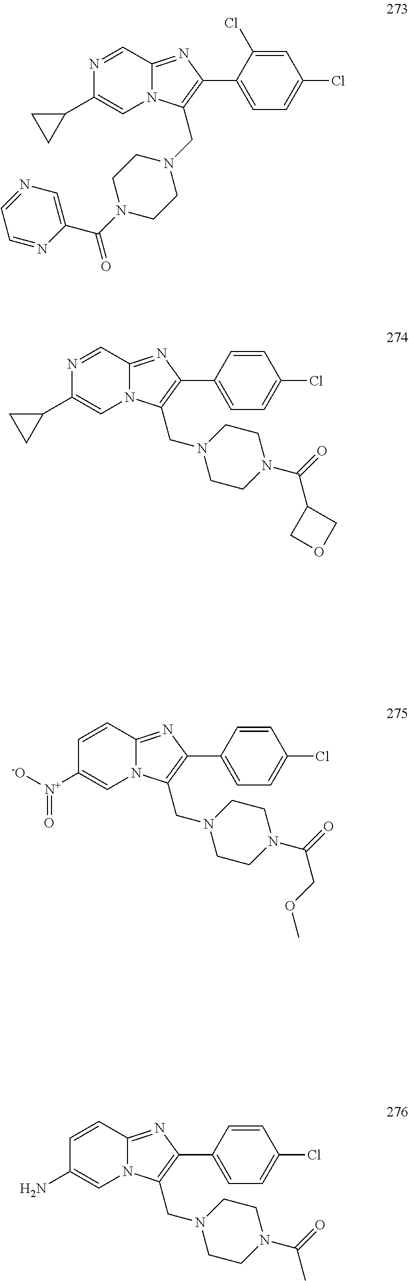

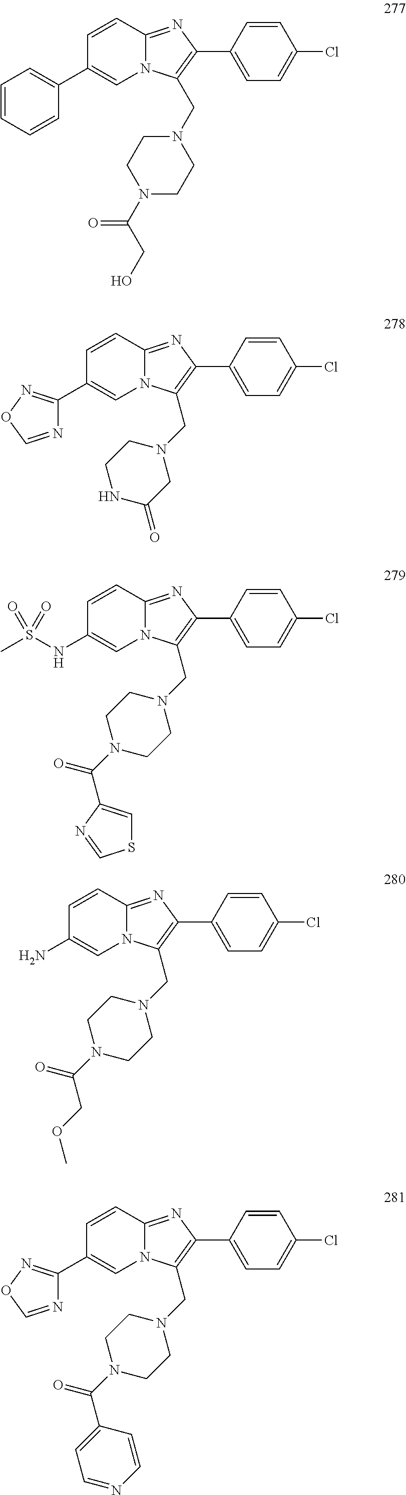

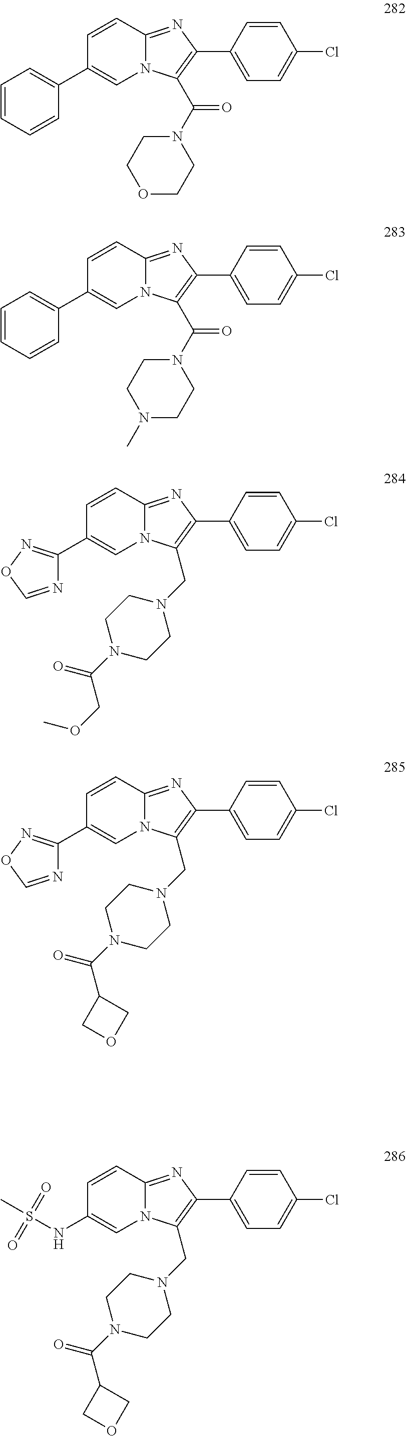

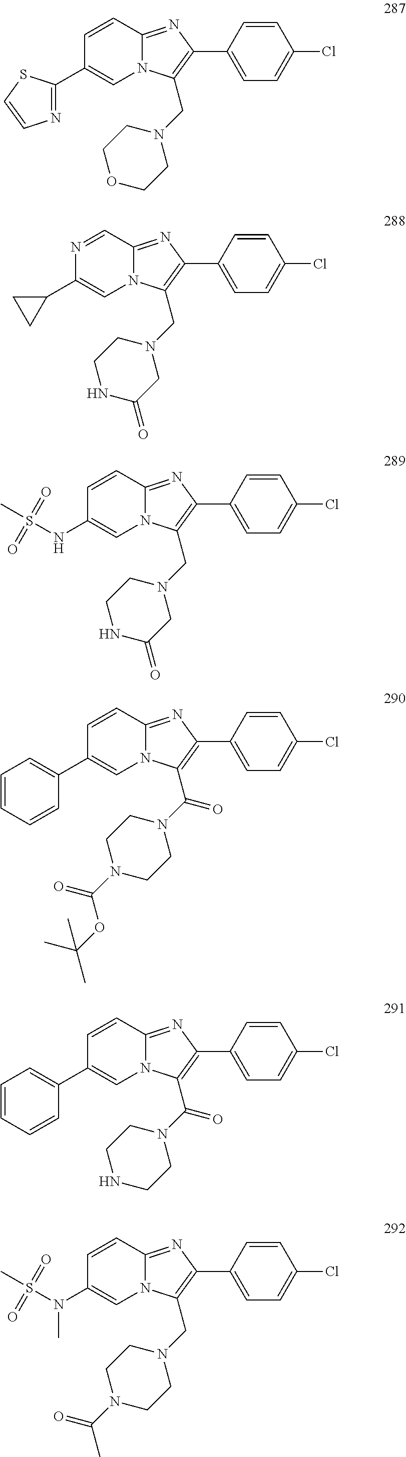

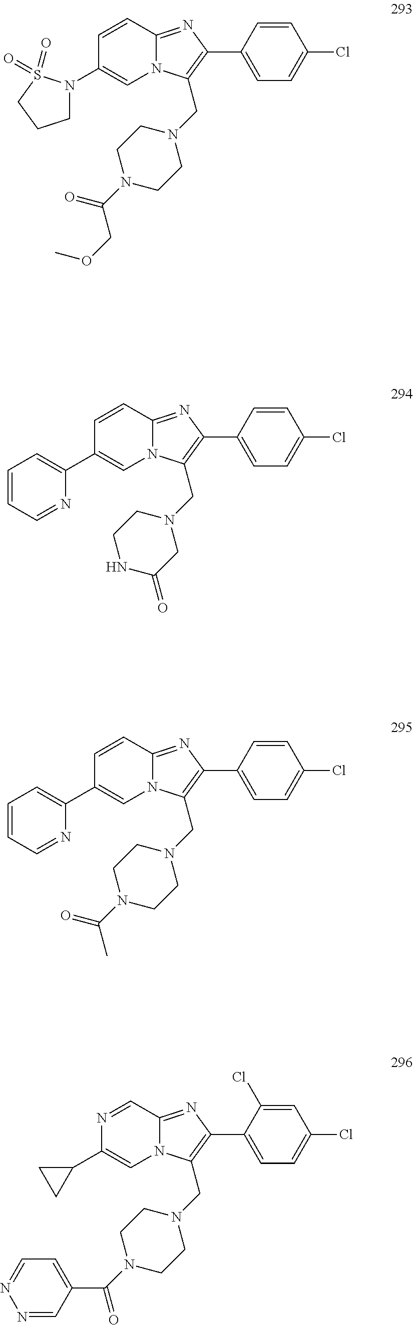 [SMILES: COCC(=O)N1CCN(Cc2c(-c3ccc(Cl)cc3)nc3ccc(N4CCCS4(=O)=O)cn23)CC1 ; O=C1CN(Cc2c(-c3ccc(Cl)cc3)nc3ccc(-c4ccccn4)cn23)CCN1 ; CC(=O)N1CCN(Cc2c(-c3ccc(Cl)cc3)nc3ccc(-c4ccccn4)cn23)CC1 ; O=C(c1ccnnc1)N1CCN(Cc2c(-c3ccc(Cl)cc3Cl)nc3cnc(C4CC4)cn23)CC1]

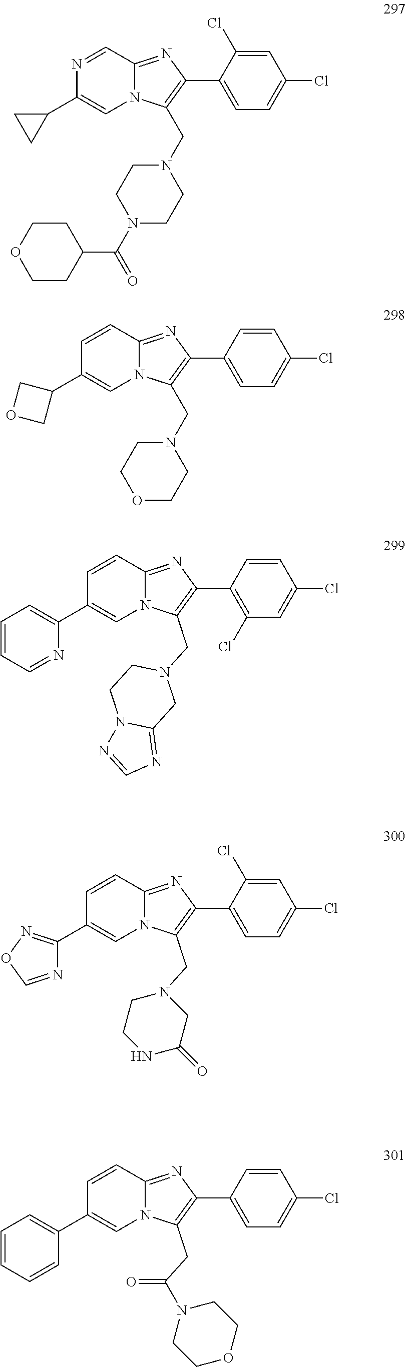 [SMILES: O=C(C1CCOCC1)N1CCN(Cc2c(-c3ccc(Cl)cc3Cl)nc3cnc(C4CC4)cn23)CC1 ; Clc1ccc(-c2nc3ccc(C4COC4)cn3c2CN2CCOCC2)cc1 ; Clc1ccc(-c2nc3ccc(-c4ccccn4)cn3c2CN2CCn3ncnc3C2)c(Cl)c1 ; O=C1CN(Cc2c(-c3ccc(Cl)cc3Cl)nc3ccc(-c4ncon4)cn23)CCN1 ; O=C(Cc1c(-c2ccc(Cl)cc2)nc2ccc(-c3ccccc3)cn12)N1CCOCC1]

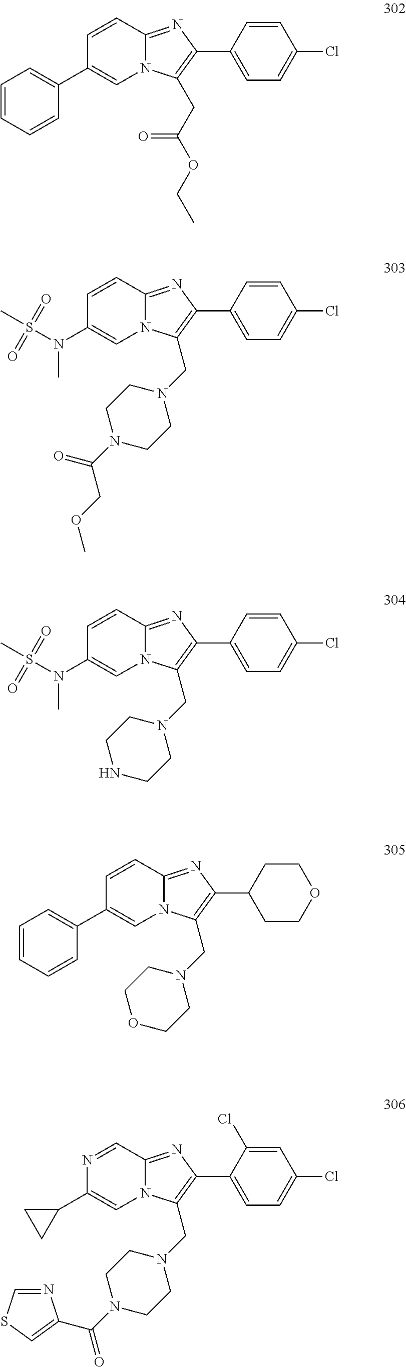 [SMILES: CCOC(=O)Cc1c(-c2ccc(Cl)cc2)nc2ccc(-c3ccccc3)cn12 ; COCC(=O)N1CCN(Cc2c(-c3ccc(Cl)cc3)nc3ccc(N(C)S(C)(=O)=O)cn23)CC1 ; CN(c1ccc2nc(-c3ccc(Cl)cc3)c(CN3CCNCC3)n2c1)S(C)(=O)=O ; c1ccc(-c2ccc3nc(C4CCOCC4)c(CN4CCOCC4)n3c2)cc1 ; O=C(c1cscn1)N1CCN(Cc2c(-c3ccc(Cl)cc3Cl)nc3cnc(C4CC4)cn23)CC1]

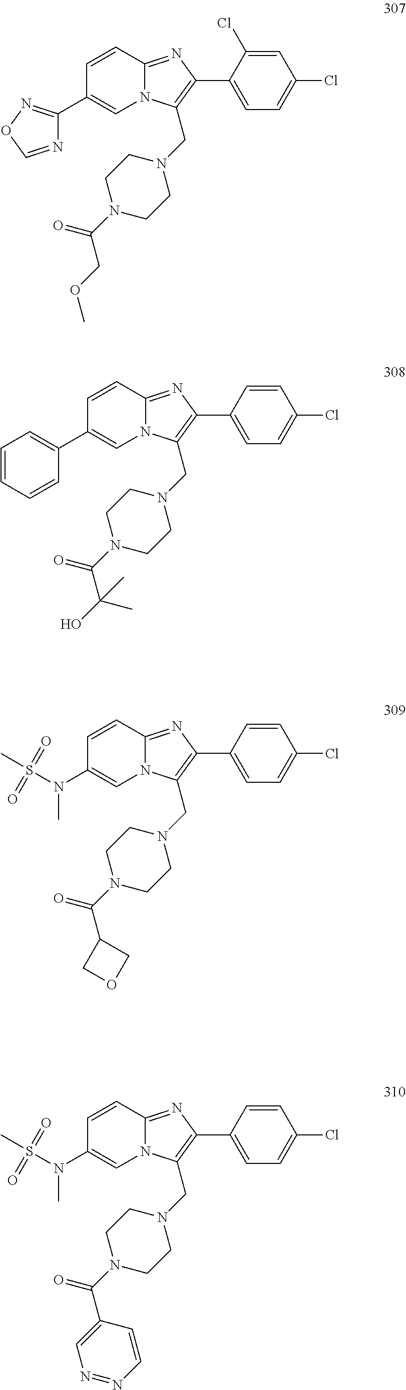 [SMILES: COCC(=O)N1CCN(Cc2c(-c3ccc(Cl)cc3Cl)nc3ccc(-c4ncon4)cn23)CC1 ; CC(C)(O)C(=O)N1CCN(Cc2c(-c3ccc(Cl)cc3)nc3ccc(-c4ccccc4)cn23)CC1 ; CN(c1ccc2nc(-c3ccc(Cl)cc3)c(CN3CCN(C(=O)C4COC4)CC3)n2c1)S(C)(=O)=O ; CN(c1ccc2nc(-c3ccc(Cl)cc3)c(CN3CCN(C(=O)c4ccnnc4)CC3)n2c1)S(C)(=O)=O]

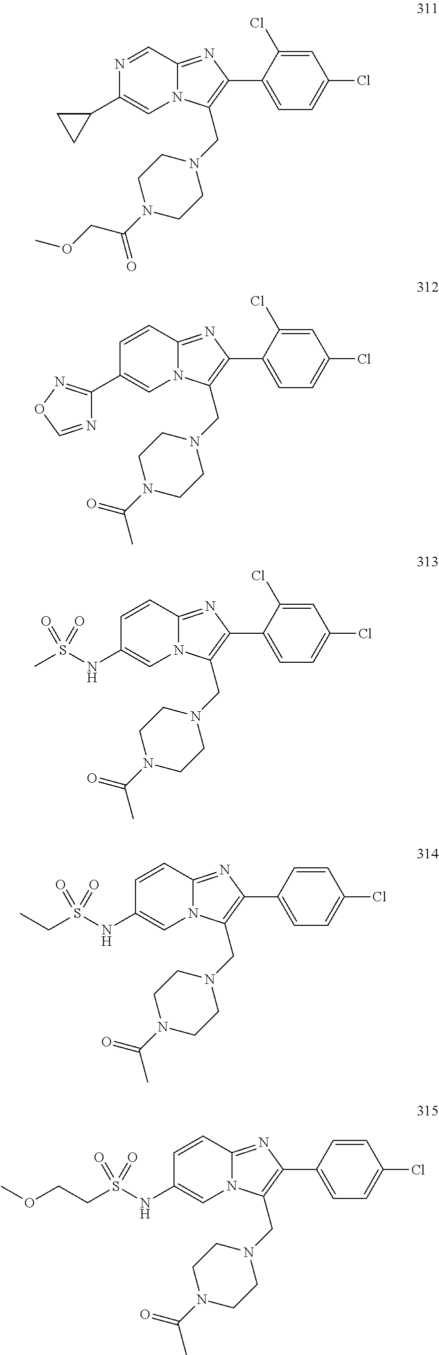 [SMILES: COCC(=O)N1CCN(Cc2c(-c3ccc(Cl)cc3Cl)nc3cnc(C4CC4)cn23)CC1 ; CC(=O)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(NS(C)(=O)=O)cn23)CC1 ; CCS(=O)(=O)Nc1ccc2nc(-c3ccc(Cl)cc3)c(CN3CCN(C(C)=O)CC3)n2c1 ; COCCS(=O)(=O)Nc1ccc2nc(-c3ccc(Cl)cc3)c(CN3CCN(C(C)=O)CC3)n2c1]

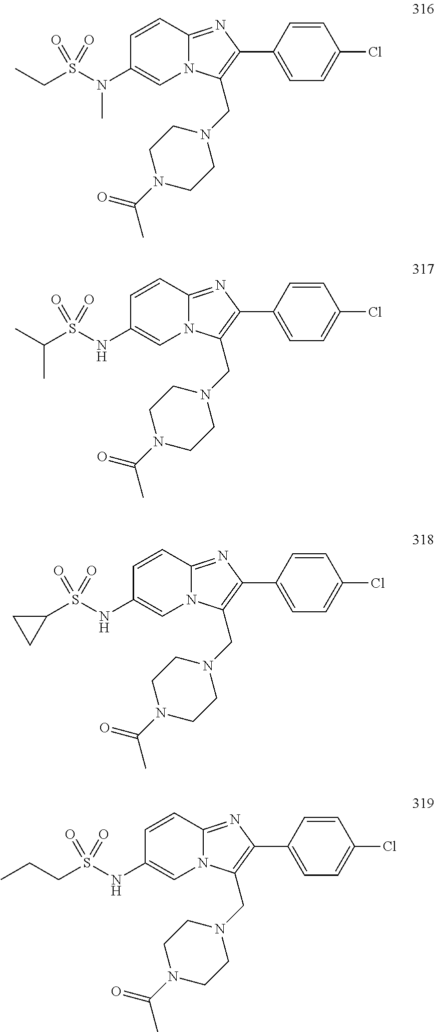

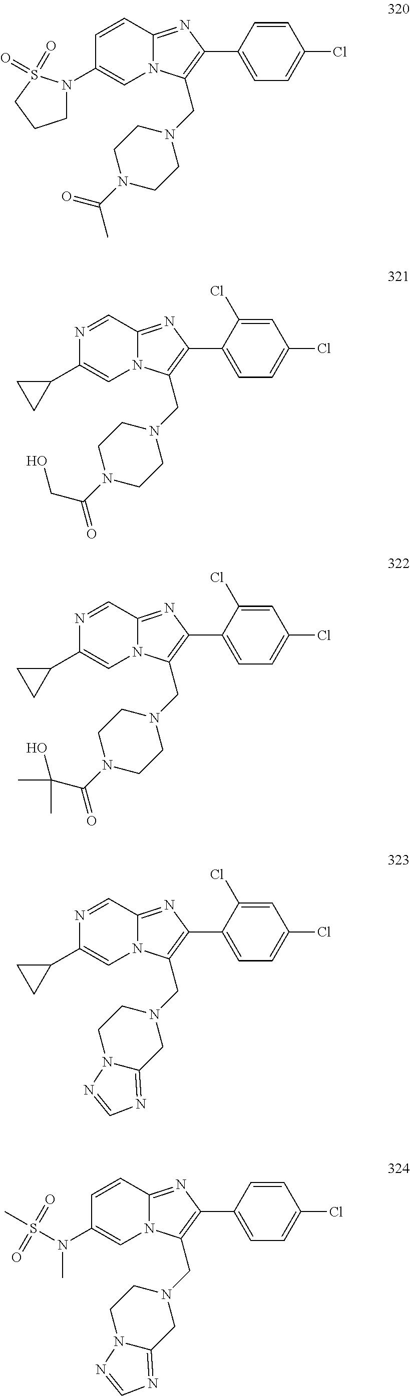 [SMILES: CC(=O)N1CCN(Cc2c(-c3ccc(Cl)cc3)nc3ccc(N4CCCS4(=O)=O)cn23)CC1 ; O=C(CO)N1CCN(Cc2c(-c3ccc(Cl)cc3Cl)nc3cnc(C4CC4)cn23)CC1 ; CC(C)(O)C(=O)N1CCN(Cc2c(-c3ccc(Cl)cc3Cl)nc3cnc(C4CC4)cn23)CC1 ; Clc1ccc(-c2nc3cnc(C4CC4)cn3c2CN2CCn3ncnc3C2)c(Cl)c1 ; CN(c1ccc2nc(-c3ccc(Cl)cc3)c(CN3CCn4ncnc4C3)n2c1)S(C)(=O)=O]

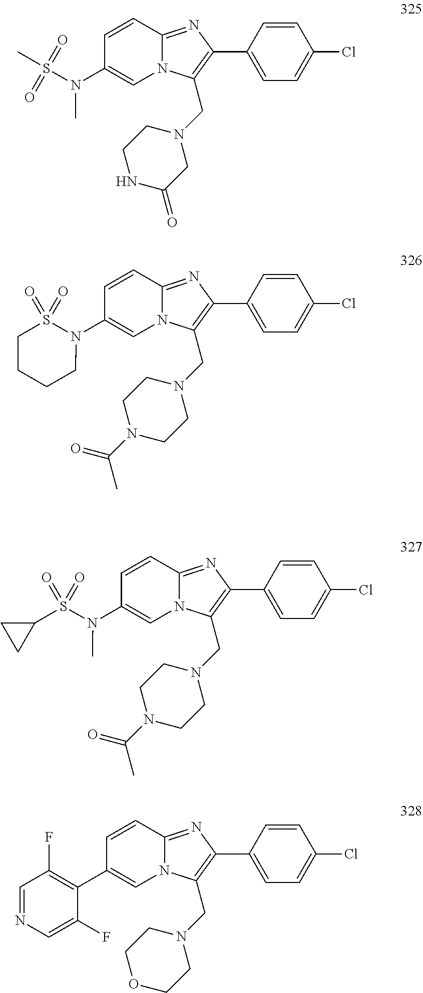

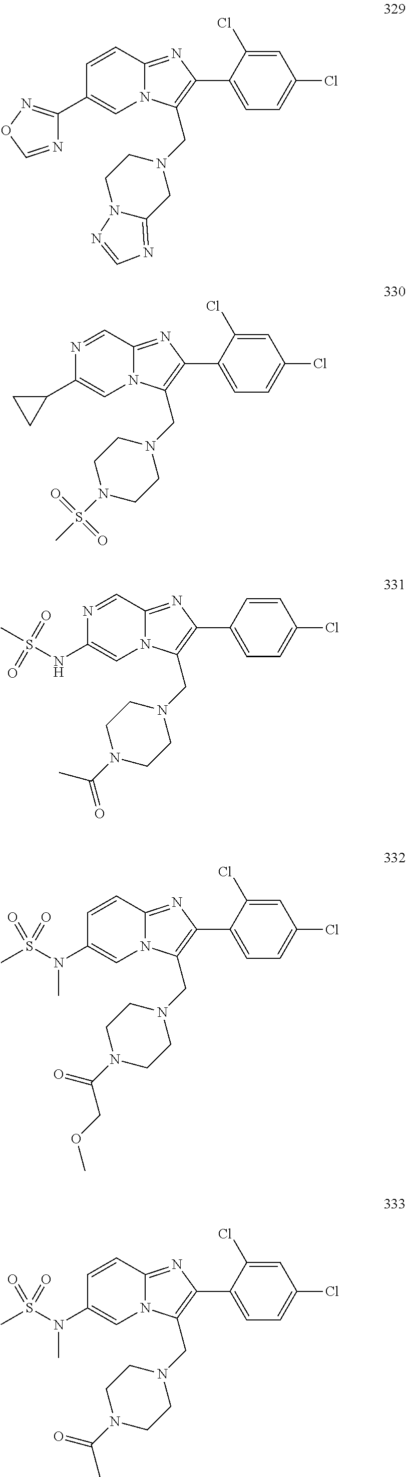 [SMILES: Clc1ccc(-c2nc3ccc(-c4ncon4)cn3c2CN2CCn3ncnc3C2)c(Cl)c1 ; CS(=O)(=O)N1CCN(Cc2c(-c3ccc(Cl)cc3Cl)nc3cnc(C4CC4)cn23)CC1 ; CC(=O)N1CCN(Cc2c(-c3ccc(Cl)cc3)nc3cnc(NS(C)(=O)=O)cn23)CC1 ; COCC(=O)N1CCN(Cc2c(-c3ccc(Cl)cc3Cl)nc3ccc(N(C)S(C)(=O)=O)cn23)CC1 ; CC(=O)N1CCN(Cc2c(-c3ccc(Cl)cc3Cl)nc3ccc(N(C)S(C)(=O)=O)cn23)CC1]

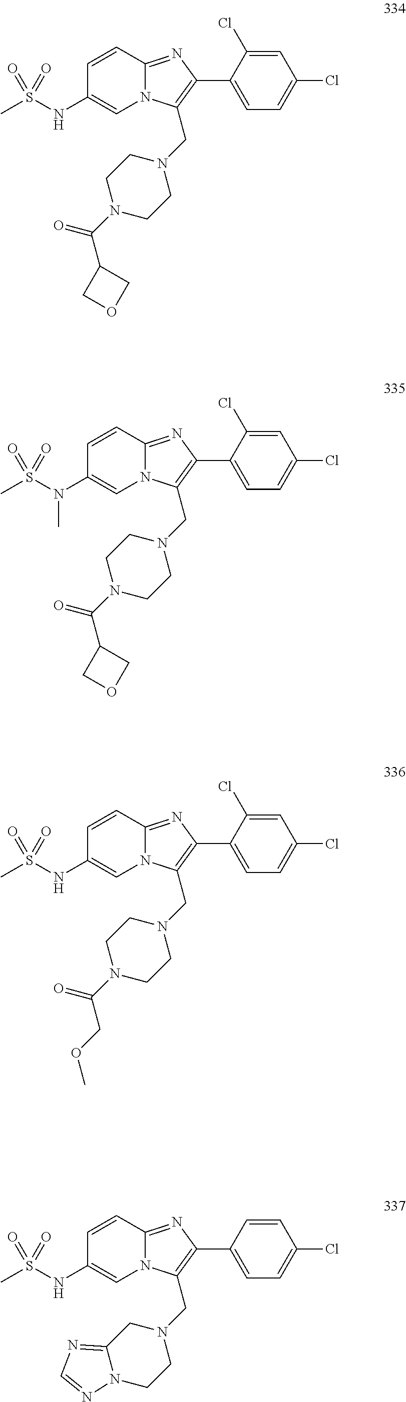 [SMILES: CS(=O)(=O)Nc1ccc2nc(-c3ccc(Cl)cc3Cl)c(CN3CCN(C(=O)C4COC4)CC3)n2c1 ; CN(c1ccc2nc(-c3ccc(Cl)cc3Cl)c(CN3CCN(C(=O)C4COC4)CC3)n2c1)S(C)(=O)=O ; COCC(=O)N1CCN(Cc2c(-c3ccc(Cl)cc3Cl)nc3ccc(NS(C)(=O)=O)cn23)CC1 ; CS(=O)(=O)Nc1ccc2nc(-c3ccc(Cl)cc3)c(CN3CCn4ncnc4C3)n2c1]

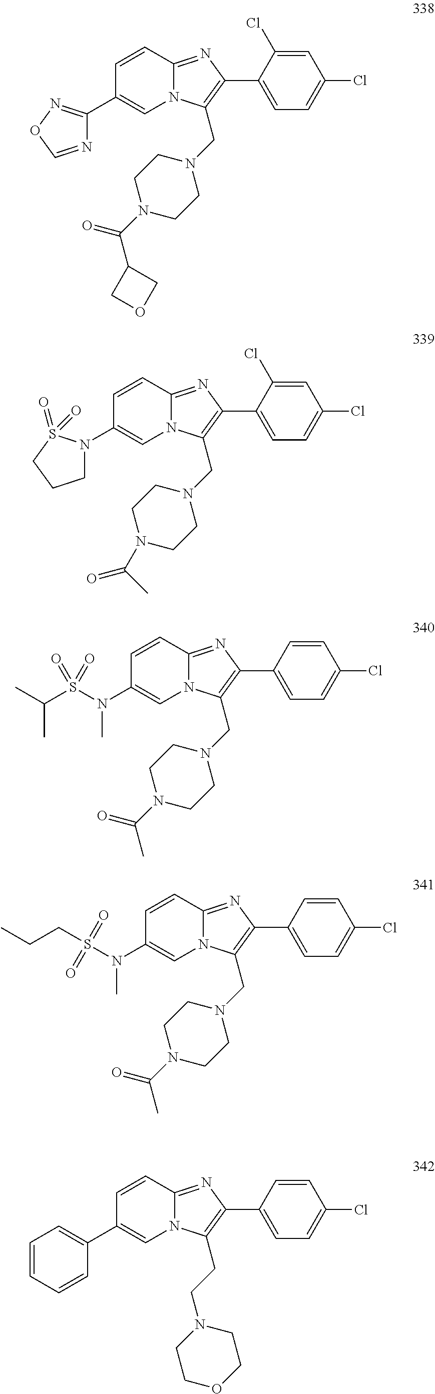

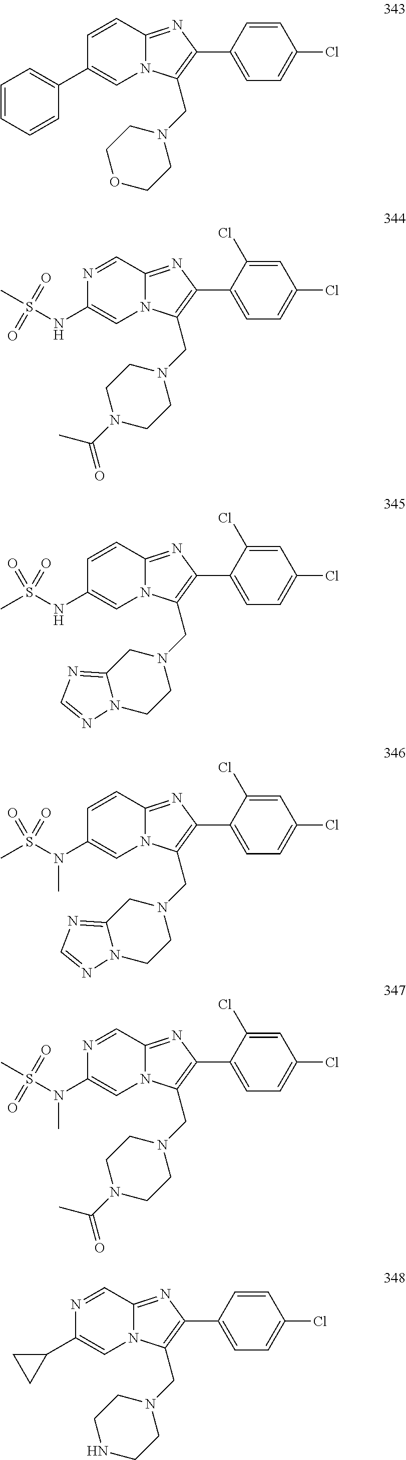

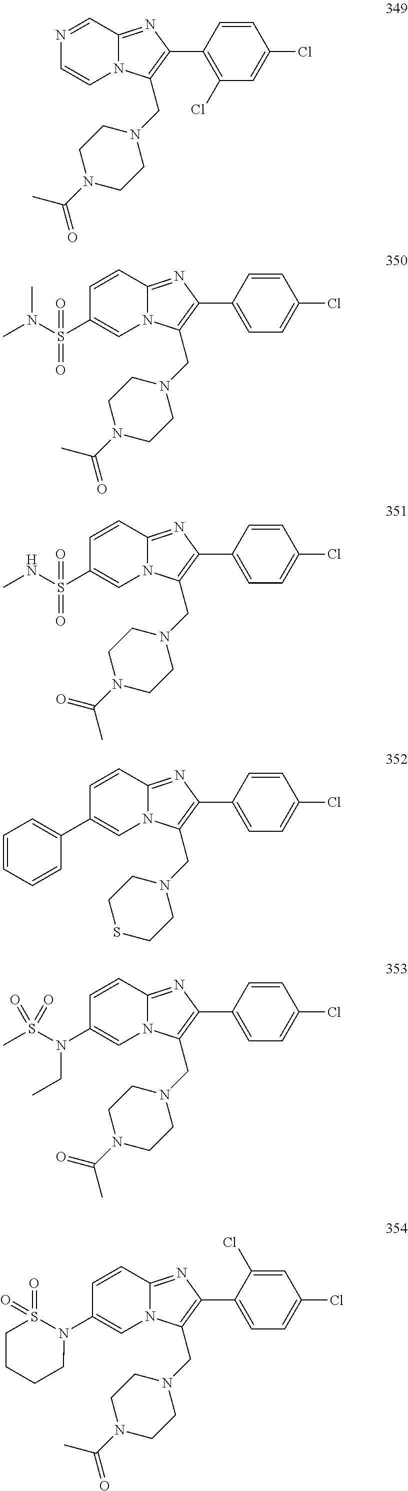

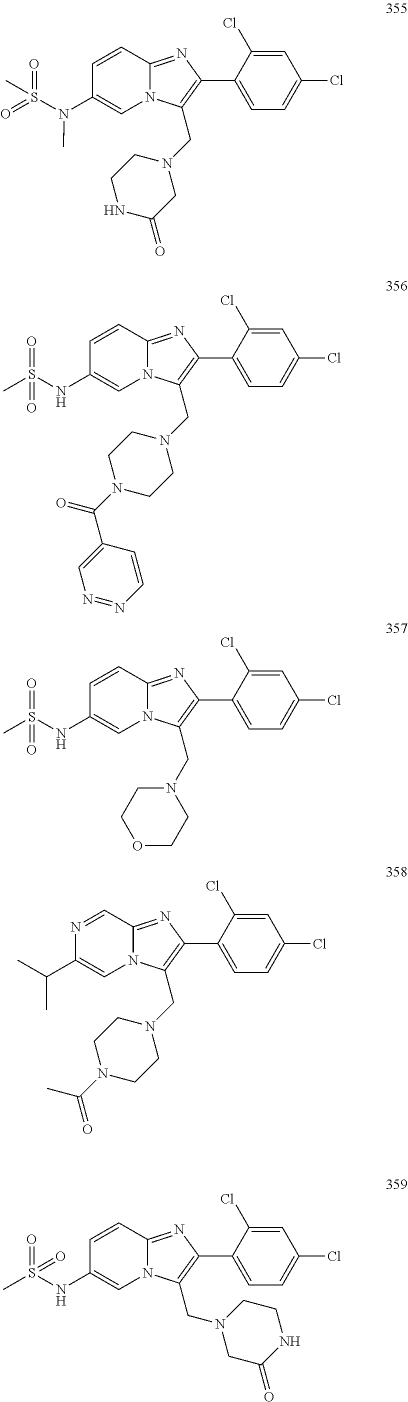 [SMILES: CN(c1ccc2nc(-c3ccc(Cl)cc3Cl)c(CN3CCNC(=O)C3)n2c1)S(C)(=O)=O ; CS(=O)(=O)Nc1ccc2nc(-c3ccc(Cl)cc3Cl)c(CN3CCN(C(=O)c4ccnnc4)CC3)n2c1 ; CS(=O)(=O)Nc1ccc2nc(-c3ccc(Cl)cc3Cl)c(CN3CCOCC3)n2c1 ; CC(=O)N1CCN(Cc2c(-c3ccc(Cl)cc3Cl)nc3cnc(C(C)C)cn23)CC1 ; CS(=O)(=O)Nc1ccc2nc(-c3ccc(Cl)cc3Cl)c(CN3CCNC(=O)C3)n2c1]

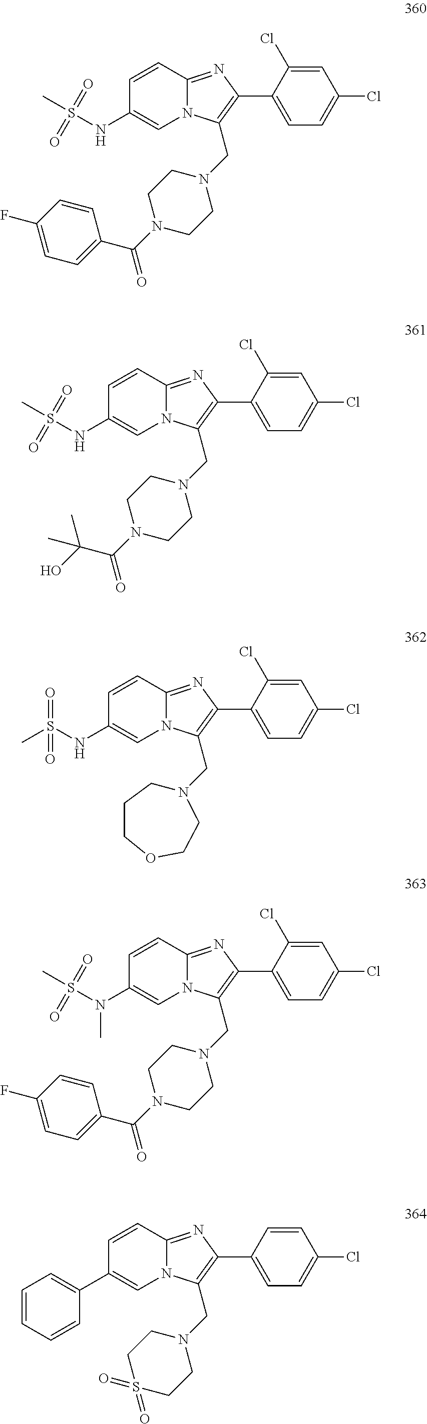 [SMILES: CS(=O)(=O)Nc1ccc2nc(-c3ccc(Cl)cc3Cl)c(CN3CCN(C(=O)c4ccc(F)cc4)CC3)n2c1 ; CC(C)(O)C(=O)N1CCN(Cc2c(-c3ccc(Cl)cc3Cl)nc3ccc(NS(C)(=O)=O)cn23)CC1 ; CS(=O)(=O)Nc1ccc2nc(-c3ccc(Cl)cc3Cl)c(CN3CCCOCC3)n2c1 ; CN(c1ccc2nc(-c3ccc(Cl)cc3Cl)c(CN3CCN(C(=O)c4ccc(F)cc4)CC3)n2c1)S(C)(=O)=O ; O=S1(=O)CCN(Cc2c(-c3ccc(Cl)cc3)nc3ccc(-c4ccccc4)cn23)CC1]

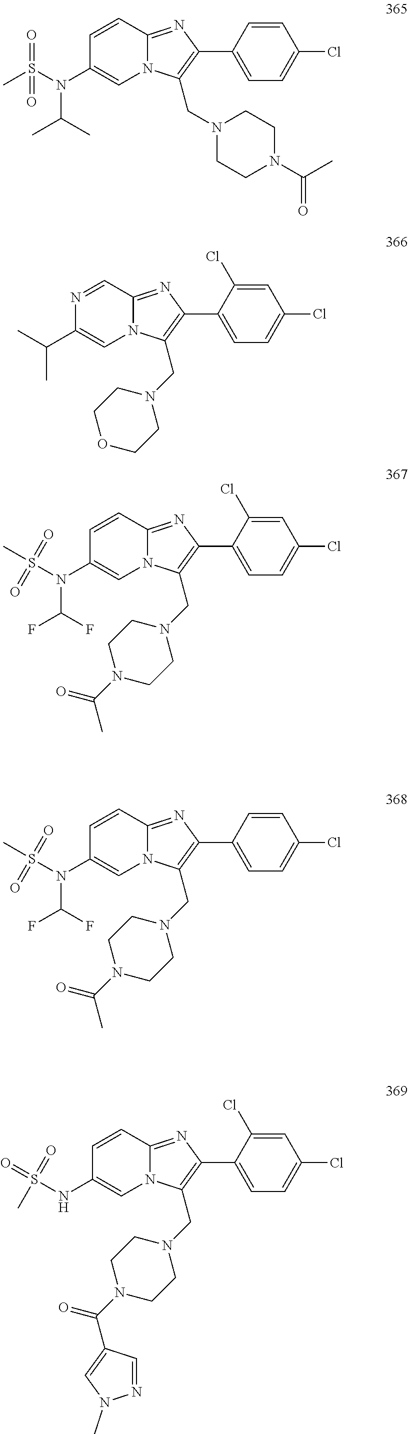

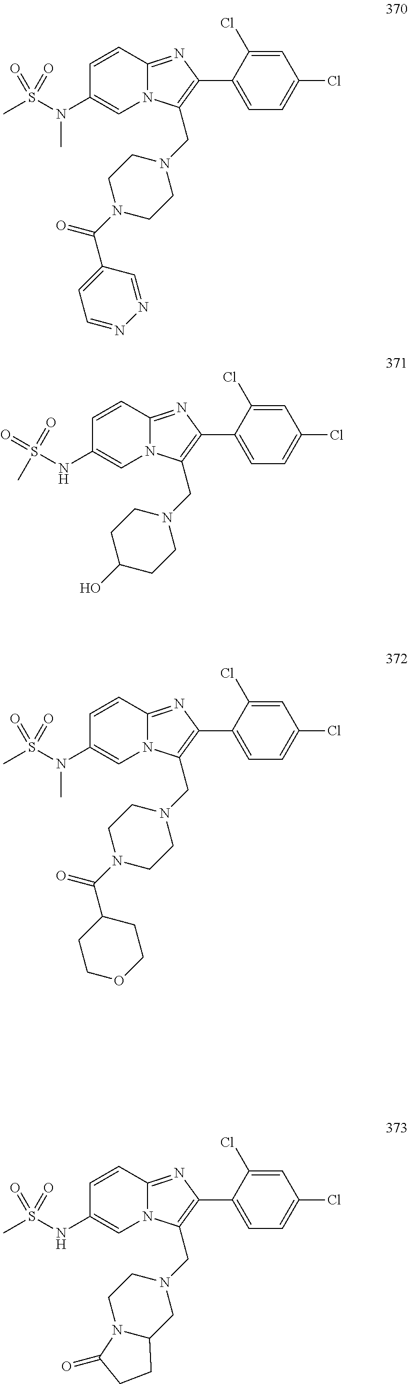 [SMILES: CN(c1ccc2nc(-c3ccc(Cl)cc3Cl)c(CN3CCN(C(=O)c4ccnnc4)CC3)n2c1)S(C)(=O)=O ; CS(=O)(=O)Nc1ccc2nc(-c3ccc(Cl)cc3Cl)c(CN3CCC(O)CC3)n2c1 ; CN(c1ccc2nc(-c3ccc(Cl)cc3Cl)c(CN3CCN(C(=O)C4CCOCC4)CC3)n2c1)S(C)(=O)=O ; CS(=O)(=O)Nc1ccc2nc(-c3ccc(Cl)cc3Cl)c(CN3CCN4C(=O)CCC4C3)n2c1]

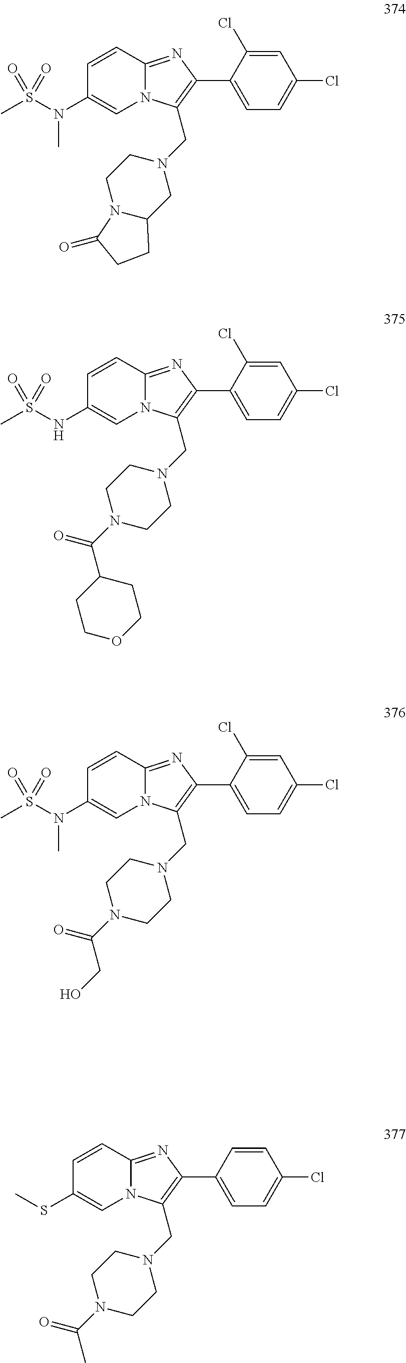 [SMILES: CN(c1ccc2nc(-c3ccc(Cl)cc3Cl)c(CN3CCN4C(=O)CCC4C3)n2c1)S(C)(=O)=O ; CS(=O)(=O)Nc1ccc2nc(-c3ccc(Cl)cc3Cl)c(CN3CCN(C(=O)C4CCOCC4)CC3)n2c1 ; CN(c1ccc2nc(-c3ccc(Cl)cc3Cl)c(CN3CCN(C(=O)CO)CC3)n2c1)S(C)(=O)=O ; CSc1ccc2nc(-c3ccc(Cl)cc3)c(CN3CCN(C(C)=O)CC3)n2c1]

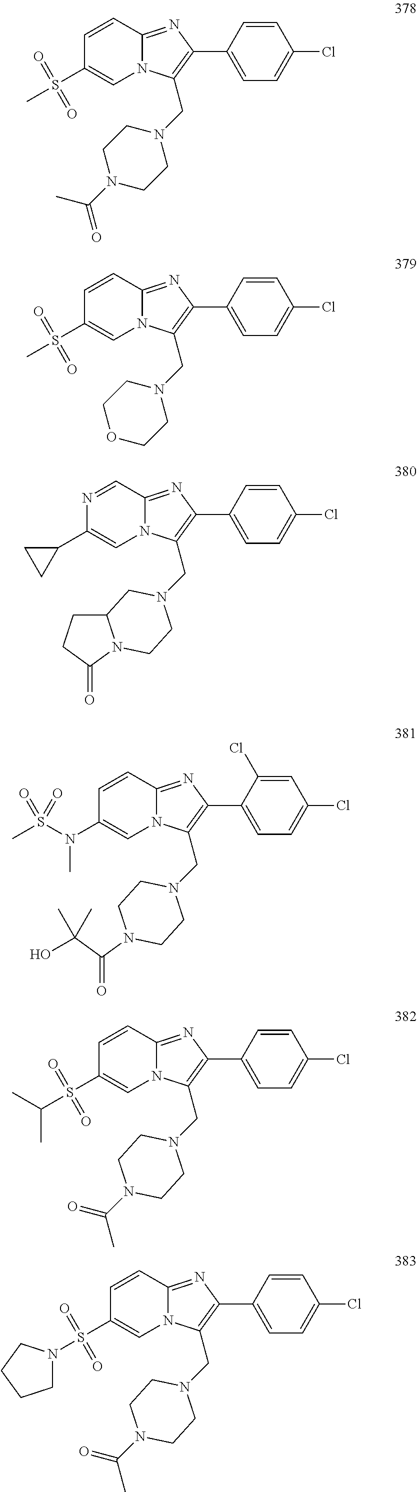

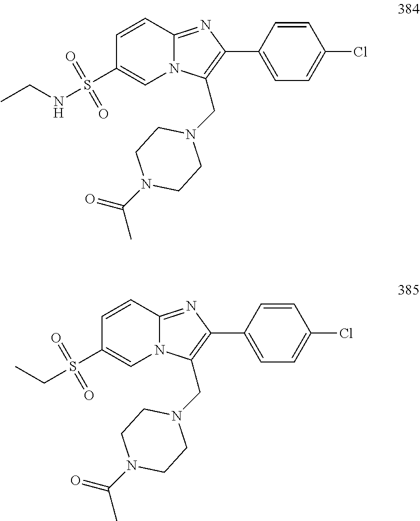 [SMILES: CCNS(=O)(=O)c1ccc2nc(-c3ccc(Cl)cc3)c(CN3CCN(C(C)=O)CC3)n2c1 ; CCS(=O)(=O)c1ccc2nc(-c3ccc(Cl)cc3)c(CN3CCN(C(C)=O)CC3)n2c1]

18. A compound for use according to claim 8, which compound comprises:
an isolated enantiomer, or
a mixture of two or more enantiomers, or
a mixture of two or more diastereomers, and/or epimers, or
a racemic mixture.

19. A method for treating a cancer, an inflammatory condition, an autoimmune condition, a neurological condition, a proliferative disorder, and/or a metabolic condition, in a patient comprising administering the compound of claim 8 to the patient.

20. The method according to claim 19, wherein the cancer is a cancer selected from a solid or liquid tumour and a cancer wherein basal glucose transport is up-regulated, including cancer of the eye, brain (such as gliomas, glioblastomas, medullablastomas, craniopharyngioma, ependymoma, and astrocytoma), spinal cord, kidney, mouth, lip, throat, oral cavity, nasal cavity, small intestine, colon, parathyroid gland, gall bladder, head and neck, breast, bone, bile duct, cervix, heart, hypopharyngeal gland, lung, bronchus, liver, skin, ureter, urethra, testicles, vagina, anus, laryngeal gland, ovary, thyroid, oesophagus, nasopharyngeal gland, pituitary gland, salivary gland, prostate, pancreas, adrenal glands; an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia, juvenile myelomonocytic leukaemia, Hodgkin lymphoma, non-Hodgkin lymphoma, mantle lymphoma, follicular lymphoma, primary effusion lymphoma, AIDS-related lymphoma, Hodgkin lymphoma, diffuse B cell lymphoma, Burkitt lymphoma, and cutaneous T-cell lymphoma).

21. The method according to claim 19, wherein the inflammatory condition and/or the autoimmune condition are conditions relating to immune B cell and/or T cell dysregulation including aberrant activation.

22. The method according to claim 21, wherein the condition relating to immune B cell and/or T cell dysregulation is selected from an inflammatory or autoimmune condition selected from gout, idiopathic pulmonary fibrosis, liver fibrosis, liver cirrhosis, polycystic kidney disease, allergic asthma, acute or chronic idiopathic inflammatory arthritis, osteoarthritis, rheumatoid arthritis, psoriasis, chronic dermatosis, myositis, a demyelinating disease, chronic obstructive pulmonary disease, interstitial lung disease, glomerulonephritis, interstitial nephritis, chronic infectious disease (such as chronic active hepatitis), Crohn's disease, ulcerative colitis, plaque formation in atherosclerosis, a degenerative disease of the joints or nervous system, multiple sclerosis, type I and type II diabetes, celiac disease, acute kidney injury, sepsis, acute liver failure, chronic liver failure, chronic kidney failure, pancreatitis, Grave's disease, psoriasis, septicemia, cystic fibrosis, meningitis and acute respiratory distress syndrome, ankylosing spondylitis, Chagas disease, dermatomyositis, endometriosis, Goodpasture's syndrome, Guillain-Barré syndrome, Hashiomoto's disease, hidradenitis suppurativa, Kawasaki disease, idiopathic thrombocytopenic purpura, lupus (such as systemic lupus erythematosus, discoid lupus, drug-induced lupus, neonatal lupus, and subacute cutaneous lupus), neuromyotonia, pemphigus vulgaris, pernicious anemia, psoriasis, polymyositis, relapsing polychondritis, scleroderma, Sjögren's syndrome, stiff person syndrome, temporal arteritis (also known as giant cell arteritis), vasculitis, vitiligo, Wegener's granulomatosis, organ transplant rejection, uveoretinitis, glomerulonephritis, allergic reaction, fibromyalgia, multiple endocrine failure, Schmidt's syndrome, autoimmune uveitis, Addison's disease, adrenalitis, thyroiditis, autoimmune thyroid disease, gastric atrophy, chronic hepatitis, lupoid hepatitis, atherosclerosis, hypoparathyroidism, Dressler's syndrome, autoimmune thrombocytopenia, idiopathic thrombocytopenic purpura, hemolytic anemia, atopic dermatitis, dermatitis herpetiformis, alopecia arcata, pemphigoid, progressive systemic sclerosis, CREST syndrome (calcinosis, Raynaud's phenomenon, esophageal dysmotility, and sclerodactyl), polyarteritis nedosa, atopic rhinitis, sarcoidosis, rheumatic fever, erythema multiforme, Cushing's syndrome, toxic epidermal necrolysis, alveolitis, allergic alveolitis, fibrosing alveolitis, interstitial lung disease, Takayasu's arteritis, polymyalgia rheumatica, schistosomiasis, ascariasis, aspergillosis, Sampter's syndrome, lymphomatoid granulomatosis, Caplan's syndrome, dengue, encephalomyelitis, endocarditis, endomyocardial fibrosis, endophthalmitis, erythema elevatum et diutinum, erythroblastosis fetalis, eosinophilic faciitis, Shulman's syndrome, Felty's syndrome, filariasis, cyclitis, chronic cyclitis, heterochronic cyclitis, Fuch's cyclitis, Henoch-Schonlein purpura, Eaton-Lambert syndrome, cryoglobulinemia, Waldenstrom's macroglobulemia, and Evan's syndrome.

23. The method according to claim 19, wherein the proliferative disorder is a proliferative disorder selected from diseases of benign, pre-malignant, and malignant cellular proliferation, including neoplasms and tumours (such as histocytoma, glioma, astrocyoma, and osteoma), cancers, leukemias, psoriasis, bone diseases, fibroproliferative disorders (such as those of connective tissues), idiopathic pulmonary fibrosis, polycystic kidney disease, renal cyst formation, intimal hyperplasia, chronic liver disease, liver fibrosis, liver cirrhosis, scleroderma, restenosis and atherosclerosis.

24. The method according to claim 19, wherein the neurological condition is a neurological condition selected from epilepsy, schizophrenia, Alzheimer's disease, Parkinson's disease, Huntington's disease, amyotrophic lateral sclerosis (Lou Gehrig's Disease), frontotemporal dementia, Lewy body dementia, vascular dementia, progressive supranuclear palsy, corticobasal degeneration and multiple system atrophy.

25. The method according to claim 19, wherein the metabolic condition is a metabolic condition selected from metabolic syndrome, obesity, diabetes (such as diabetes type I, diabetes type II, MODY, and gestational diabetes), pre-diabetes, lipodystrophy, impaired glucose tolerance, elevated plasma insulin concentrations, insulin resistance, dyslipidemia, hyperglycemia, hyperlipidemia, hypertriglyceridemia, hypertension, cardiovascular disease or respiratory conditions, hyperphagia, hypophagia, triglyceride storage disease, Bardet-Biedl syndrome, Lawrence-Moon syndrome, Prader-Labhart-Willi syndrome, Kearns-Sayre syndrome, medium chain acyl-CoA dehydrogenase deficiency and cachexia.

26. A pharmaceutical composition comprising a compound as defined in claim 18.

27. A pharmaceutical composition according to claim 26, further comprising a pharmaceutically acceptable additive and/or excipient.

28. A pharmaceutical composition according to claim 26, which composition is used to treat a cancer, an inflammatory condition, an autoimmune condition, a neurological condition, a proliferative disorder, and/or a metabolic condition in a patient.

29. A pharmaceutical composition according to claim 28 for treating a cancer, further comprising a further agent for treating cancer; preferably wherein the further agent for treating cancer is selected from anti-microtubule agents, platinum coordination complexes, alkylating agents, antibiotic agents, topoisomerase II inhibitors, antimetabolites, topoisomerase I inhibitors, hormones and hormone analogues, signal transduction pathway inhibitors, non-receptor tyrosine kinase angiogenesis inhibitors, immunotherapeutic agents, proapoptotic agents, respiratory chain un-couplers such as metformin, and cell cycle signalling inhibitors.

30. A method of treating a cancer and/or a condition and/or a disorder, which method comprises administering to a patient a composition as defined in claim 26.

31. The method according to claim 30, wherein the cancer is a cancer selected from a solid or liquid tumour and a cancer wherein basal glucose transport is up-regulated, including cancer of the eye, brain (such as gliomas, glioblastomas, medullablastomas, craniopharyngioma, ependymoma, and astrocytoma), spinal cord, kidney, mouth, lip, throat, oral cavity, nasal cavity, small intestine, colon, parathyroid gland, gall bladder, head and neck, breast, bone, bile duct, cervix, heart, hypopharyngeal gland, lung, bronchus, liver, skin, ureter, urethra, testicles, vagina, anus, laryngeal gland, ovary, thyroid, oesophagus, nasopharyngeal gland, pituitary gland, salivary gland, prostate, pancreas, adrenal glands; an endometrial cancer, oral cancer, melanoma, neuroblastoma, gastric cancer, an angiomatosis, a hemangioblastoma, a pheochromocytoma, a pancreatic cyst, a renal cell carcinoma, Wilms' tumour, squamous cell carcinoma, sarcoma, osteosarcoma, Kaposi sarcoma, rhabdomyosarcoma, hepatocellular carcinoma, PTEN Hamartoma-Tumor Syndromes (PHTS) (such as Lhermitte-Duclos disease, Cowden syndrome, Proteus syndrome, and Proteus-like syndrome), leukaemias and lymphomas (such as acute lymphoblastic leukaemia, chronic lymphocytic leukaemia, acute myelogenous leukaemia, chronic myelogenous leukaemia, hairy cell leukaemia, T-cell prolymphocytic leukemia (T-PLL), large granular lymphocytic leukemia, adult T-cell leukemia, juvenile myelomonocytic leukaemia, Hodgkin lymphoma, non-Hodgkin lymphoma, mantle lymphoma, follicular lymphoma, primary effusion lymphoma, AIDS-related lymphoma, Hodgkin lymphoma, diffuse B cell lymphoma, Burkitt lymphoma, and cutaneous T-cell lymphoma); and the Of condition or disorder is an inflammatory condition, an autoimmune condition, a neurological condition, a proliferative disorder, and/or a metabolic condition.

32. The method according to claim 31 for treating a cancer, which method comprises administering to a patient a further agent for treating cancer selected from anti-microtubule agents, platinum coordination complexes, alkylating agents, antibiotic agents, topoisomerase II inhibitors, antimetabolites, topoisomerase I inhibitors, hormones and hormone analogues, signal transduction pathway inhibitors, non-receptor tyrosine kinase anqioqenesis inhibitors, immunotherapeutic agents, proapoptotic agents, respiratory chain un-couplers such as metformin, and cell cycle signalling inhibitors; preferably wherein the composition and the further agent are administered simultaneously, sequentially or separately.

33. The method according to any of claim 30, wherein the patient is human.

34. A compound, which compound comprises a formula selected from one of the following: 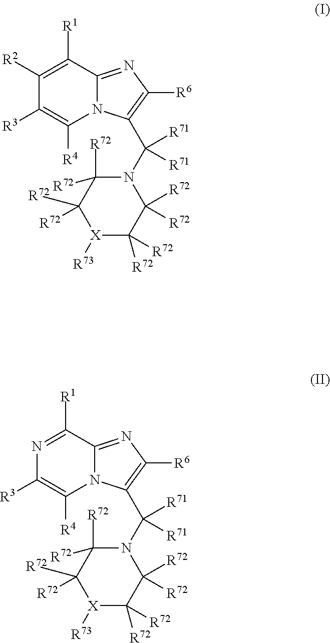
wherein in (I) the substituents may be selected from one of the following groups (i)-(vi):
(i) R³ is selected from a substituted or unsubstituted saturated carbocyclic ring having from 3 to 7 ring carbons; and a substituted or unsubstituted aromatic ring having from 3 to 7 ring carbons provided that it is not a 4-methylphenyl group; R⁶ is a substituted or unsubstituted saturated or unsaturated heterocyclic group having from 4 to 7 ring atoms; X is N; R⁷³ is a —(CO)—R⁷⁵ group; and wherein R¹, R², R⁴, R⁷¹, R⁷² and R⁷⁵ are independently selected from H and a substituted or unsubstituted organic group;
(ii) R³ is a substituted or unsubstituted saturated or unsaturated heterocyclic group having from 4 to 7 ring atoms; R⁶ is a substituted or unsubstituted saturated or unsaturated heterocyclic group having from 4 to 7 ring atoms; X is independently selected from N, O and S wherein R⁷³ is absent when its X is O or S; and wherein R¹, R², R⁴, R⁷¹, R⁷² and R⁷³ are independently selected from H and a substituted or unsubstituted organic group;
(iii) R³ is a —Z(R³³)₂ group in which Z can be C or N wherein a further H or substituted or unsubstituted organic group or R³³ group may be present when Z is C; R³³ is selected from a substituted or unsubstituted linear or branched alkyl group optionally forming a non-aromatic carbocyclic or heterocyclic ring with another R³³, a substituted sulphonyl group, and a substituted carbonyl group; provided that the —Z(R³³)₂ group is not —NH₂ or an amide bonded to the ring via a C atom; R⁶ is a substituted or unsubstituted saturated or unsaturated heterocyclic group preferably having from 4 to 7 ring atoms; X is N; and wherein R⁷³ is a —(CO)—R⁷⁵ group; and wherein R¹, R², R⁴, R⁷¹, R⁷² and R⁷⁵ are independently selected from H and a substituted or unsubstituted organic group;
(iv) R⁶ is a substituted or unsubstituted saturated homocyclic or heterocyclic group having from 3-7 ring atoms in which each ring atom is selected from C, N, O or S, provided that R⁶ is not adamantyl; X is independently selected from N, O and S wherein R⁷³ is absent when its X is O or S; and wherein R¹, R², R³, R⁴, R⁷¹, R⁷² and R⁷³ are independently selected from H and a substituted or unsubstituted organic group;
(v) groups such that the compound has a structure as follows: 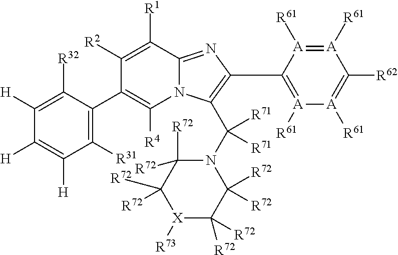
wherein R¹, R², R⁴, R⁷¹, R⁷² and R⁷³ are independently selected from H and a substituted or unsubstituted organic group; adjacent R groups may together form a substituted or unsubstituted saturated or unsaturated aliphatic or aromatic homocyclic or heterocyclic ring; A may independently be C or N wherein R⁶¹ is absent when A is N; X is independently selected from N, O and S wherein R⁷³ is absent when its X is O or S; R³¹ is selected from H and a substituted or unsubstituted organic group; R³² is selected from a substituted or unsubstituted organic group excluding H; R⁶¹ is selected from H and a substituted or unsubstituted organic group; and R⁶² is selected from a substituted or unsubstituted organic group excluding H and Me; and
(vi) groups such that the compound has a structure as follows: 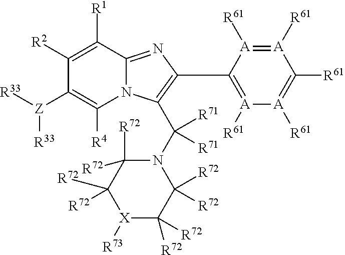
wherein R¹, R², R⁴, R⁷¹, R⁷² and R⁷³ are independently selected from H and a substituted or unsubstituted organic group; adjacent R groups may together form a substituted or unsubstituted saturated or unsaturated aliphatic or aromatic homocyclic or heterocyclic ring; each A may independently be C or N wherein R⁶¹ is absent when A is N; X is independently selected from N, O and S wherein R⁷³ is absent when its X is O or S; Z can be C or N wherein a further H or substituted or unsubstituted organic group or R³³ group may be present when Z is C; R³³ is selected from a substituted or unsubstituted linear or branched alkyl group optionally forming a non-aromatic carbocyclic or heterocyclic ring with another R³³, a substituted sulphonyl group, and a substituted carbonyl group; and R⁶¹ is selected from H and a substituted or unsubstituted organic group;
and wherein in (II) the substituents are as follows:
R¹, R³ R⁴, R⁷¹, R⁷² and R⁷³ are independently selected from H and a substituted or unsubstituted organic group, provided that R³ is not Me; adjacent R groups may together form a substituted or unsubstituted saturated or unsaturated aliphatic or aromatic homocyclic or heterocyclic ring; X is independently selected from N, O and S wherein R⁷³ is absent when its X is O or S; and R⁶ is selected from H and a substituted or unsubstituted organic group except CO₂H and CO₂Et.

35. A compound according to claim 34,
wherein R³ is H or a group selected from the following groups:
a halogen (such as F, Cl, Br and I);
a linear or branched C₁-C₆ alkyl group (such as methyl (Me), ethyl (Et), propvl (Pr), iso-propyl (i-Pr), n-butyl (n-Bu), iso-butyl (i-Bu), tert-butyl (t-Bu), pentyl and hexyl); preferably a C₃-C₆ alkyl group (such as propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃ and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂-NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3, 4, 5, or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu,
(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe),
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe,
and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3, 4, 5, or 6)-Et₂-Ph-, 2,(3, 4, 5, or 6)-Pr₂-Ph-, 2,(3, 4, 5, or 6)-Bu₂-Ph-, 2,(3, 4, 5, or 6)-(CN)₂-Ph-, 2,(3, 4, 5, or 6)-(NO₂)₂-Ph-, 2,(3, 4, 5, or 6)-(NH₂)₂-Ph-, 2,(3, 4, 5, or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂) Ph-, 3-(NH₂)-Ph-, 4-(NH₂-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R³ is selected from the following:
an unsubstituted, 2-monosubstituted, or 2,6-disubstituted phenyl group, preferably where the substituent is selected from F, Cl and CN (such as Ph-, 2-F-Ph-, 2,6-F₂-Ph-, 2-Cl-Ph-, 2,6-Cl₂-Ph-, 2-CN-Ph-, 2,6-(CN) Ph-, and 2,6-Cl, CN-Ph-);
an unsubstituted or 3-monosubstituted pyridine-2-yl, or a 2,6-disubstituted Pyridine-4-vl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂-Pyr-4-yl;
an unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl);
a substituted or unsubstituted 1,2,4-oxadiazol-3-yl group;
a cyclic aminosulphonyl- group (such as —N(SO₂)(CH₂)₃ and —N(SO₂)(CH₂)₄);
a linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe;
a linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl);
a linear or branched sulphonyl group (such as —SO₂Me, —SO₂Et, —SO₂Pr, —SO₂iPr);
a thioether group (such as —SMe, —SEt, —SPr, and SiPr); and
an isopropyl, cyclopropyl and a propen-2-yl group;
wherein R⁶ is H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt),
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH_{z}NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3, 4, 5, or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5 NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R⁶ comprises—an aromatic group selected from Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-,
particularly preferably wherein R⁶ is selected from the following:
an unsubstituted, 2-, 3- or 4-monosubstituted, and a 2,4 or 3,4-disubstituted phenyl group, preferably wherein the substituent is F, Cl or —OMe (such as 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph);
a substituted or unsubstituted pyridine-2-yl, pyridine-3-yl or pyridine-4-yl group preferably wherein the substituent is F or CI (such as pyridine-2-yl, pyridine-3-yl, Pyridine-4-yl, 3-F-pyridine-2-yl, 3-Cl-pyridine-2-yl, 4-F-pyridine-2-yl, 4-Cl-pyridine-2-yl, 5-F-pyridine-2-yl, 5-Cl-pyridine-2-yl; and
a cyclic ether group (such as tetrahydrofuran-2-yl, tetrahydrofuran-3-yl, terahydropyran-2-yl, tetrahydropyran-3-yl and tetrahydropyran-4-yl),
wherein R¹, R², and R⁴ are each independently H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH_{z}Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3, 4, 5, or 6)Cl₂-Ph, —NH-2,(3, 4, 5, or 6 Br₂-Ph, —NH-2,(3, 4, 5, or 6)I₂-Ph, —NH-2,(3, 4, 5, or 6)Me₂-Ph, —NH-2,(3, 4, 5, or 6)Et₂-Ph, —NH-2,(3, 4, 5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-Pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe,
and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3, 4, 5, or 6)-(NO₂)₂-Ph-, 2,(3, 4, 5, or 6)-(NH₂)₂-Ph-, 2,(3, 4, 5, or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-), and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl),
wherein each of R⁷¹, R⁷², R⁷³, and R⁷⁵ is independently H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5 or 6)F₂-Ph, —NH-2,(3, 4, 5, or 6)Cl₂-Ph, —NH-2,(3, 4, 5, or 6 Br₂-Ph, —NH-2,(3, 4, 5, or 6)I₂-Ph, —NH-2,(3, 4, 5, or 6)Me₂-Ph, —NH-2,(3, 4, 5, or 6)Et₂-Ph, —NH-2,(3, 4, 5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-Pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe,
and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3, 4, 5, or 6)-(NO₂)₂-Ph-, 2,(3, 4, 5, or 6)-(NH₂)₂-Ph-, 2,(3, 4, 5, or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-), and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R⁷⁵ is a substituted or unsubstituted group selected from a linear or branched alkyl group, a cycloalkyl group, a saturated or unsaturated heterocyclic group including an aromatic heterocyclic group, and an aryl group; chosen from the following:
a linear or branched C₁-C₆ alkyl group (such as Me, Et, Pr, i-Pr, n-Bu, i-Bu, t-Bu, pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, and —Cl₃);
a linear or branched primary secondary or tertiary C₁-C₆ alkylamine group (such as —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ alkoxyalkyl or aryloxyalkyl group (such as —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
an aromatic group (such as Ph-, 2-F-Ph-, 3-F-Ph-, 4-F-Ph-, 2-C₁-Ph-, 3-Cl-Ph-, 4-C₁-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3, 4, 5, or 6)-Cl₂-Ph-, 2,(3, 4, 5, or 6)-Br₂-Ph-, 2,(3, 4, 5, or 6)-I₂-Ph-, 2,(3, 4, 5, or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂) Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pvrimidin-4-yl, pyrimidin-5-yl, pyrimidin-6-yl, tetrazole-1yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
wherein two of R⁷¹ and/or two of R⁷² when attached to the same carbon atom may together form a ketone group.

36. A compound, which is a compound of any of the following formulae:

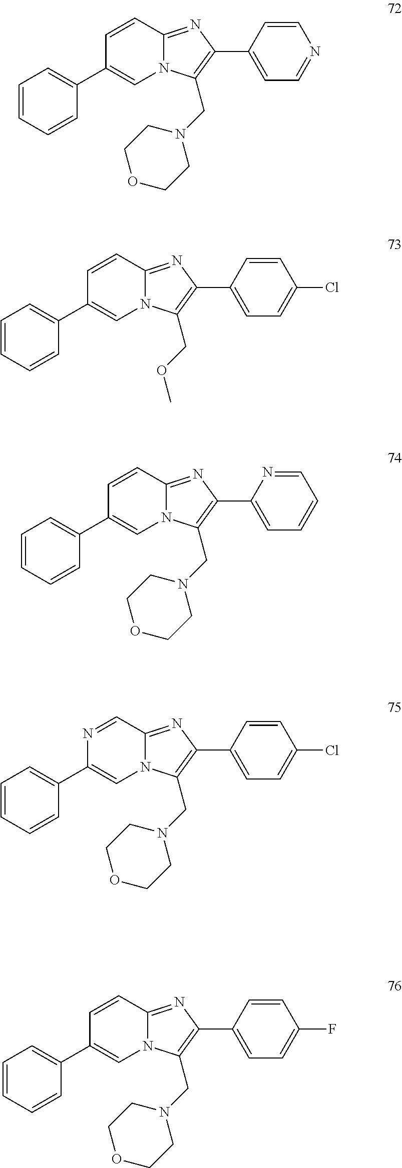 [SMILES: c1ccc(-c2ccc3nc(-c4ccncc4)c(CN4CCOCC4)n3c2)cc1 ; COCc1c(-c2ccc(Cl)cc2)nc2ccc(-c3ccccc3)cn12 ; c1ccc(-c2ccc3nc(-c4ccccn4)c(CN4CCOCC4)n3c2)cc1 ; Clc1ccc(-c2nc3cnc(-c4ccccc4)cn3c2CN2CCOCC2)cc1 ; Fc1ccc(-c2nc3ccc(-c4ccccc4)cn3c2CN2CCOCC2)cc1]

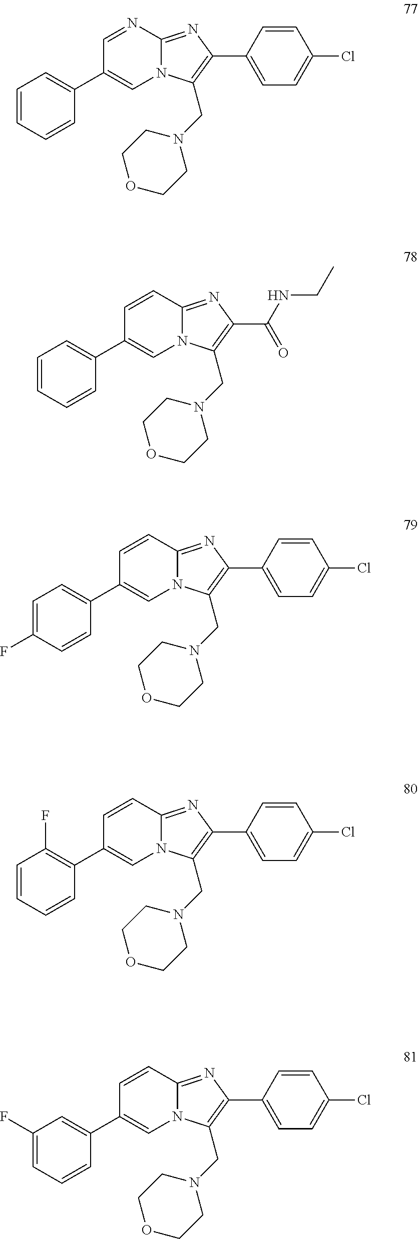 [SMILES: Clc1ccc(-c2nc3ncc(-c4ccccc4)cn3c2CN2CCOCC2)cc1 ; CCNC(=O)c1nc2ccc(-c3ccccc3)cn2c1CN1CCOCC1 ; Fc1ccc(-c2ccc3nc(-c4ccc(Cl)cc4)c(CN4CCOCC4)n3c2)cc1 ; Fc1ccccc1-c1ccc2nc(-c3ccc(Cl)cc3)c(CN3CCOCC3)n2c1 ; Fc1cccc(-c2ccc3nc(-c4ccc(Cl)cc4)c(CN4CCOCC4)n3c2)c1]

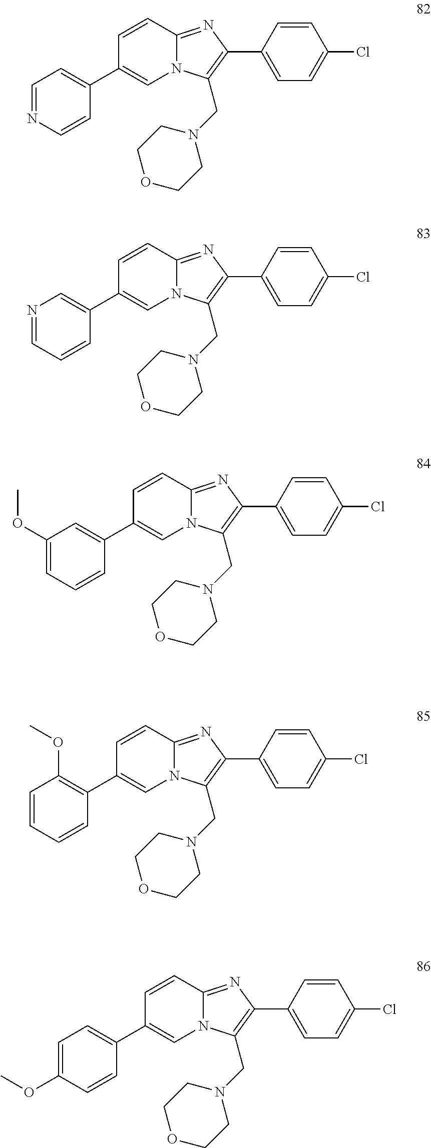 [SMILES: Clc1ccc(-c2nc3ccc(-c4ccncc4)cn3c2CN2CCOCC2)cc1 ; Clc1ccc(-c2nc3ccc(-c4cccnc4)cn3c2CN2CCOCC2)cc1 ; COc1cccc(-c2ccc3nc(-c4ccc(Cl)cc4)c(CN4CCOCC4)n3c2)c1 ; COc1ccccc1-c1ccc2nc(-c3ccc(Cl)cc3)c(CN3CCOCC3)n2c1 ; COc1ccc(-c2ccc3nc(-c4ccc(Cl)cc4)c(CN4CCOCC4)n3c2)cc1]

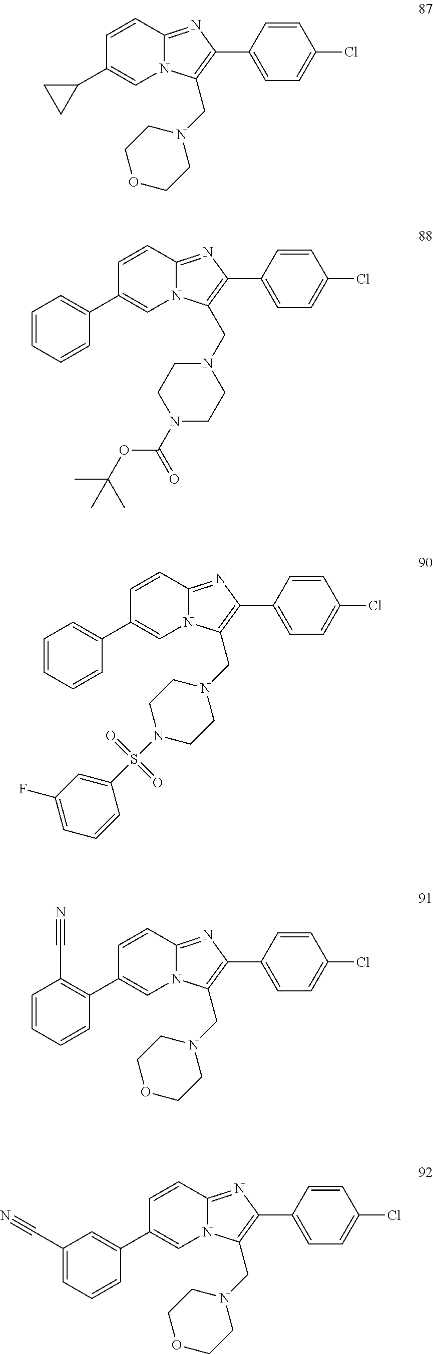 [SMILES: Clc1ccc(-c2nc3ccc(C4CC4)cn3c2CN2CCOCC2)cc1 ; CC(C)(C)OC(=O)N1CCN(Cc2c(-c3ccc(Cl)cc3)nc3ccc(-c4ccccc4)cn23)CC1 ; O=S(=O)(c1cccc(F)c1)N1CCN(Cc2c(-c3ccc(Cl)cc3)nc3ccc(-c4ccccc4)cn23)CC1 ; N#Cc1ccccc1-c1ccc2nc(-c3ccc(Cl)cc3)c(CN3CCOCC3)n2c1 ; N#Cc1cccc(-c2ccc3nc(-c4ccc(Cl)cc4)c(CN4CCOCC4)n3c2)c1]

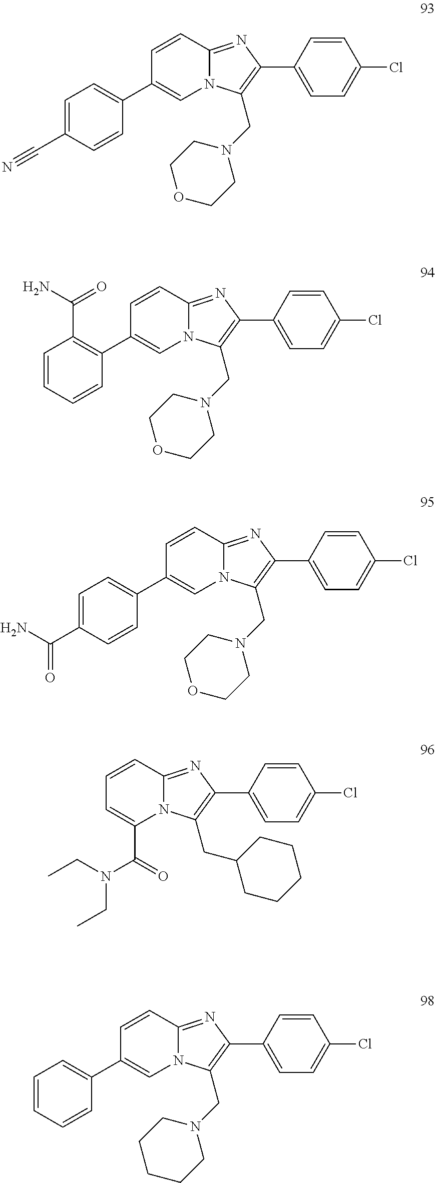 [SMILES: N#Cc1ccc(-c2ccc3nc(-c4ccc(Cl)cc4)c(CN4CCOCC4)n3c2)cc1 ; NC(=O)c1ccccc1-c1ccc2nc(-c3ccc(Cl)cc3)c(CN3CCOCC3)n2c1 ; NC(=O)c1ccc(-c2ccc3nc(-c4ccc(Cl)cc4)c(CN4CCOCC4)n3c2)cc1 ; CCN(CC)C(=O)c1cccc2nc(-c3ccc(Cl)cc3)c(CC3CCCCC3)n12 ; Clc1ccc(-c2nc3ccc(-c4ccccc4)cn3c2CN2CCCCC2)cc1]

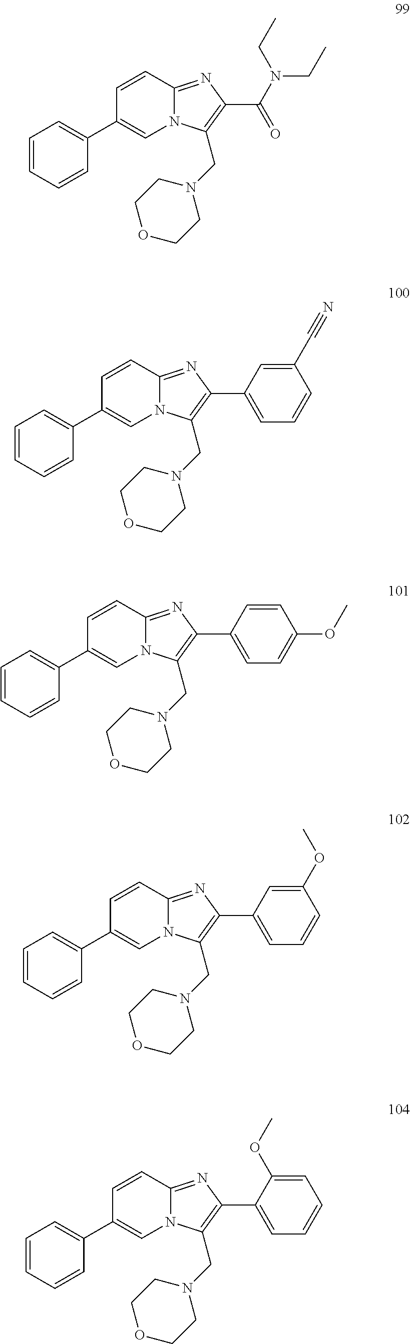 [SMILES: CCN(CC)C(=O)c1nc2ccc(-c3ccccc3)cn2c1CN1CCOCC1 ; N#Cc1cccc(-c2nc3ccc(-c4ccccc4)cn3c2CN2CCOCC2)c1 ; COc1ccc(-c2nc3ccc(-c4ccccc4)cn3c2CN2CCOCC2)cc1 ; COc1cccc(-c2nc3ccc(-c4ccccc4)cn3c2CN2CCOCC2)c1 ; COc1ccccc1-c1nc2ccc(-c3ccccc3)cn2c1CN1CCOCC1]

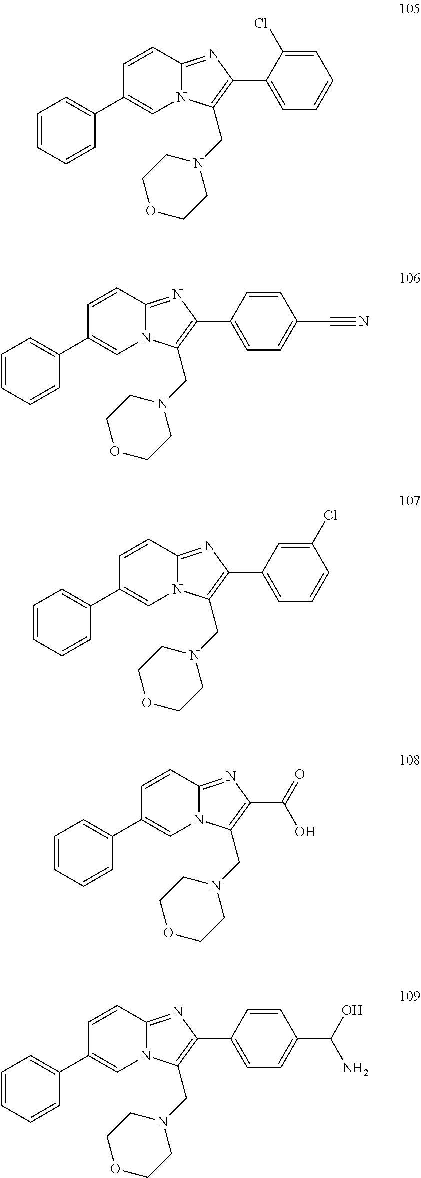 [SMILES: Clc1ccccc1-c1nc2ccc(-c3ccccc3)cn2c1CN1CCOCC1 ; N#Cc1ccc(-c2nc3ccc(-c4ccccc4)cn3c2CN2CCOCC2)cc1 ; Clc1cccc(-c2nc3ccc(-c4ccccc4)cn3c2CN2CCOCC2)c1 ; O=C(O)c1nc2ccc(-c3ccccc3)cn2c1CN1CCOCC1 ; NC(O)c1ccc(-c2nc3ccc(-c4ccccc4)cn3c2CN2CCOCC2)cc1]

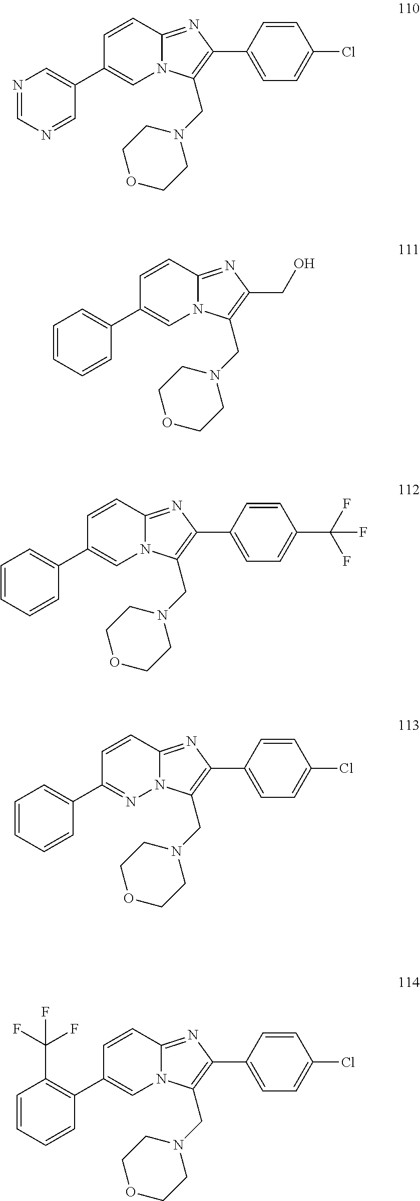 [SMILES: Clc1ccc(-c2nc3ccc(-c4cncnc4)cn3c2CN2CCOCC2)cc1 ; OCc1nc2ccc(-c3ccccc3)cn2c1CN1CCOCC1 ; FC(F)(F)c1ccc(-c2nc3ccc(-c4ccccc4)cn3c2CN2CCOCC2)cc1 ; Clc1ccc(-c2nc3ccc(-c4ccccc4)nn3c2CN2CCOCC2)cc1 ; FC(F)(F)c1ccccc1-c1ccc2nc(-c3ccc(Cl)cc3)c(CN3CCOCC3)n2c1]

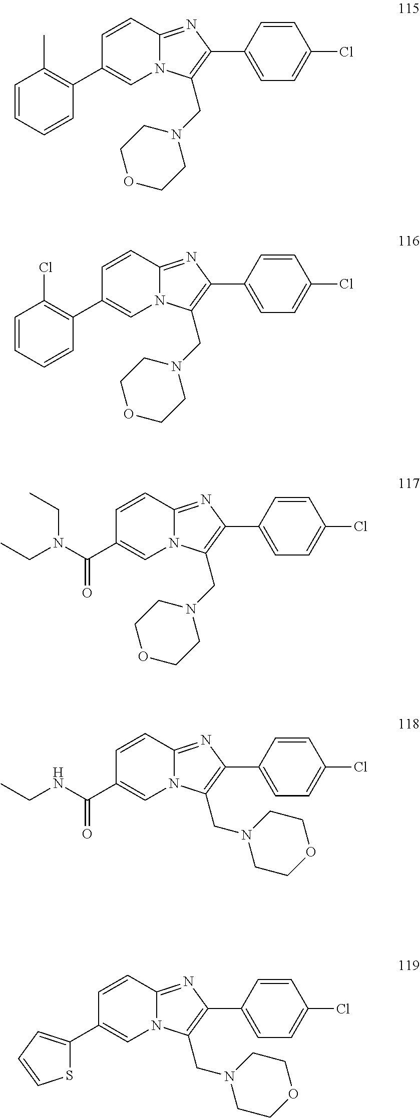 [SMILES: Cc1ccccc1-c1ccc2nc(-c3ccc(Cl)cc3)c(CN3CCOCC3)n2c1 ; Clc1ccc(-c2nc3ccc(-c4ccccc4Cl)cn3c2CN2CCOCC2)cc1 ; CCN(CC)C(=O)c1ccc2nc(-c3ccc(Cl)cc3)c(CN3CCOCC3)n2c1 ; CCNC(=O)c1ccc2nc(-c3ccc(Cl)cc3)c(CN3CCOCC3)n2c1 ; Clc1ccc(-c2nc3ccc(-c4cccs4)cn3c2CN2CCOCC2)cc1]

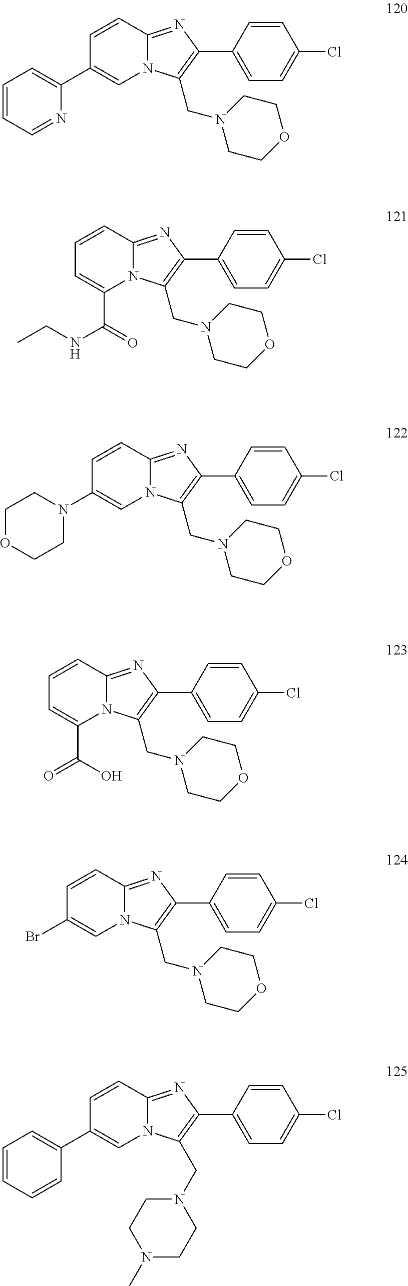 [SMILES: Clc1ccc(-c2nc3ccc(-c4ccccn4)cn3c2CN2CCOCC2)cc1 ; CCNC(=O)c1cccc2nc(-c3ccc(Cl)cc3)c(CN3CCOCC3)n12 ; Clc1ccc(-c2nc3ccc(N4CCOCC4)cn3c2CN2CCOCC2)cc1 ; O=C(O)c1cccc2nc(-c3ccc(Cl)cc3)c(CN3CCOCC3)n12 ; Clc1ccc(-c2nc3ccc(Br)cn3c2CN2CCOCC2)cc1 ; CN1CCN(Cc2c(-c3ccc(Cl)cc3)nc3ccc(-c4ccccc4)cn23)CC1]

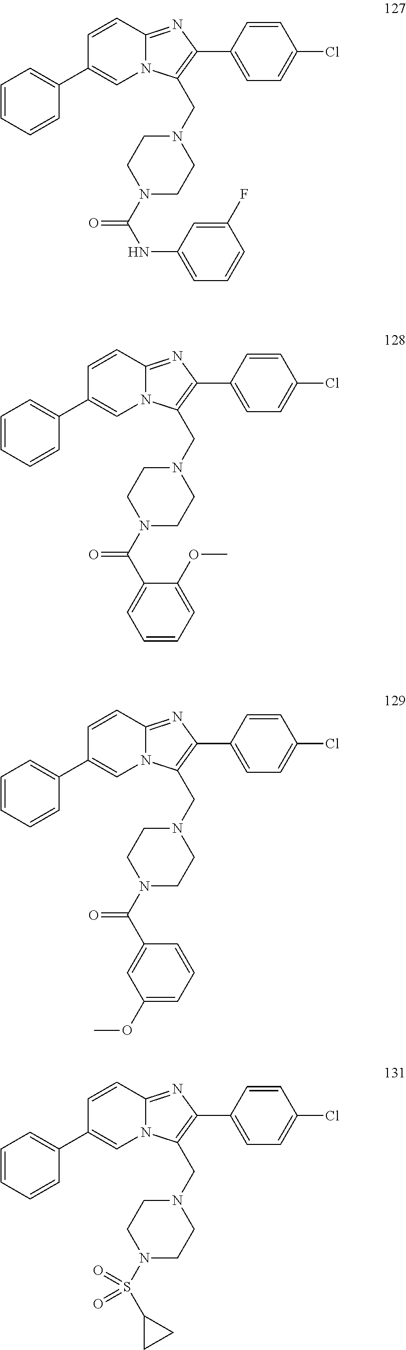 [SMILES: O=C(Nc1cccc(F)c1)N1CCN(Cc2c(-c3ccc(Cl)cc3)nc3ccc(-c4ccccc4)cn23)CC1 ; COc1ccccc1C(=O)N1CCN(Cc2c(-c3ccc(Cl)cc3)nc3ccc(-c4ccccc4)cn23)CC1 ; COc1cccc(C(=O)N2CCN(Cc3c(-c4ccc(Cl)cc4)nc4ccc(-c5ccccc5)cn34)CC2)c1 ; O=S(=O)(C1CC1)N1CCN(Cc2c(-c3ccc(Cl)cc3)nc3ccc(-c4ccccc4)cn23)CC1]

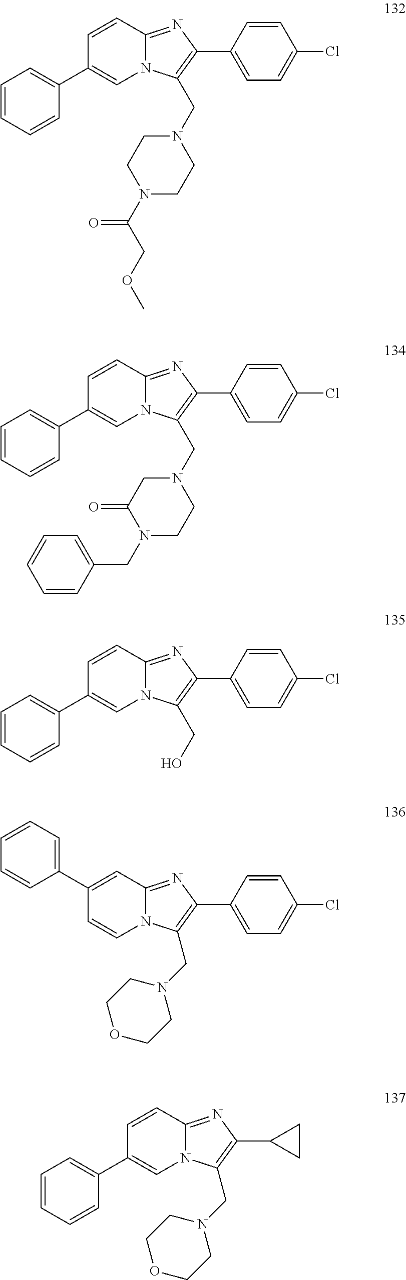 [SMILES: COCC(=O)N1CCN(Cc2c(-c3ccc(Cl)cc3)nc3ccc(-c4ccccc4)cn23)CC1 ; O=C1CN(Cc2c(-c3ccc(Cl)cc3)nc3ccc(-c4ccccc4)cn23)CCN1Cc1ccccc1 ; OCc1c(-c2ccc(Cl)cc2)nc2ccc(-c3ccccc3)cn12 ; Clc1ccc(-c2nc3cc(-c4ccccc4)ccn3c2CN2CCOCC2)cc1 ; c1ccc(-c2ccc3nc(C4CC4)c(CN4CCOCC4)n3c2)cc1]

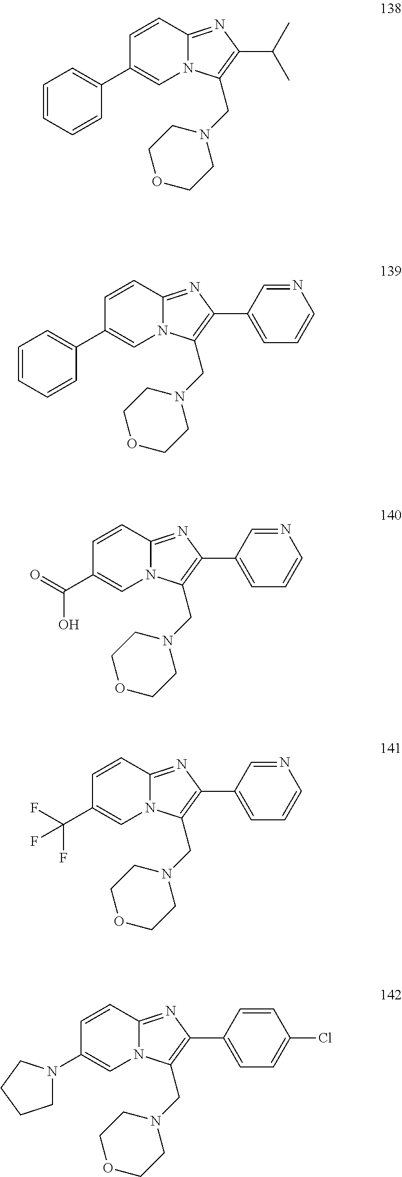 [SMILES: CC(C)c1nc2ccc(-c3ccccc3)cn2c1CN1CCOCC1 ; c1ccc(-c2ccc3nc(-c4cccnc4)c(CN4CCOCC4)n3c2)cc1 ; O=C(O)c1ccc2nc(-c3cccnc3)c(CN3CCOCC3)n2c1 ; FC(F)(F)c1ccc2nc(-c3cccnc3)c(CN3CCOCC3)n2c1 ; Clc1ccc(-c2nc3ccc(N4CCCC4)cn3c2CN2CCOCC2)cc1]

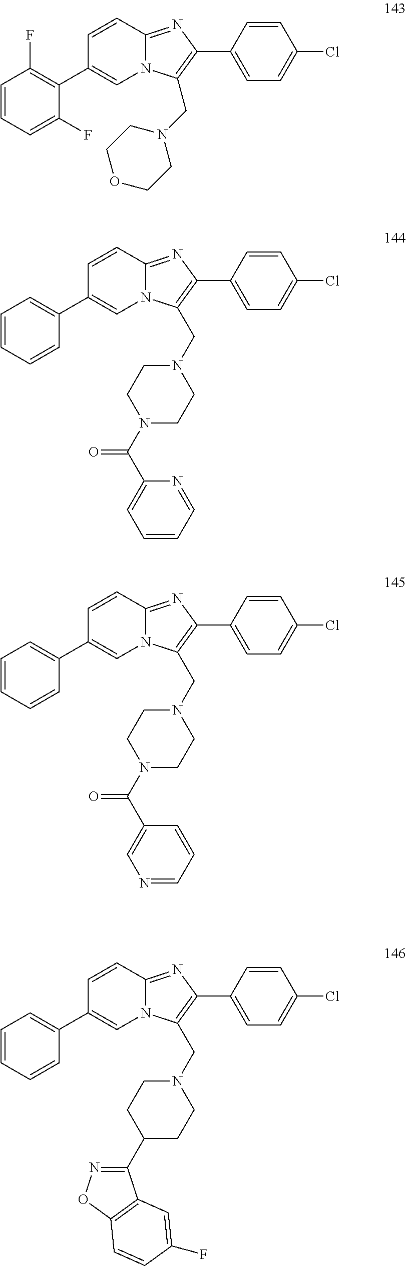 [SMILES: Fc1cccc(F)c1-c1ccc2nc(-c3ccc(Cl)cc3)c(CN3CCOCC3)n2c1 ; O=C(c1ccccn1)N1CCN(Cc2c(-c3ccc(Cl)cc3)nc3ccc(-c4ccccc4)cn23)CC1 ; O=C(c1cccnc1)N1CCN(Cc2c(-c3ccc(Cl)cc3)nc3ccc(-c4ccccc4)cn23)CC1 ; Fc1ccc2onc(C3CCN(Cc4c(-c5ccc(Cl)cc5)nc5ccc(-c6ccccc6)cn45)CC3)c2c1]

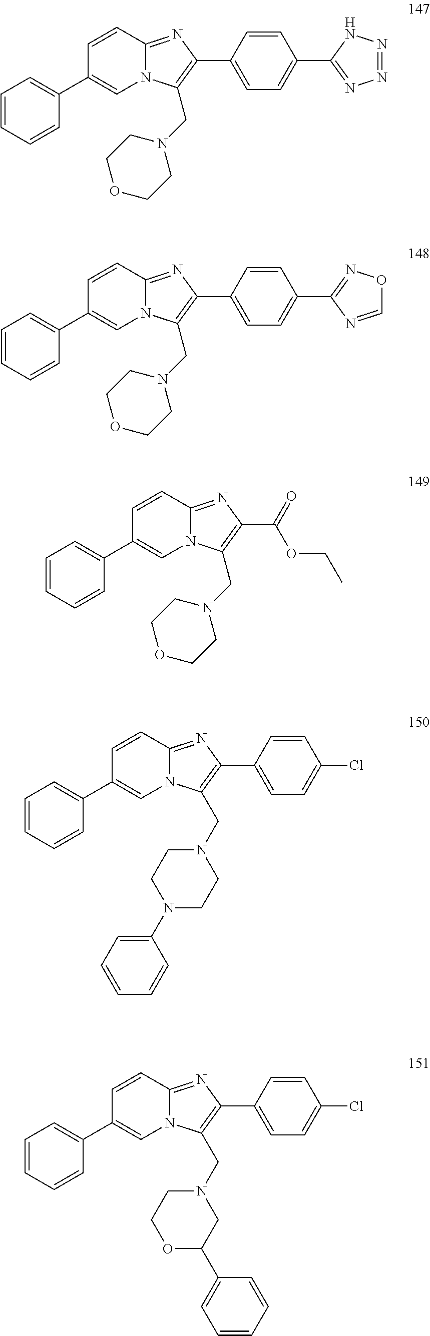 [SMILES: c1ccc(-c2ccc3nc(-c4ccc(-c5nnn[nH]5)cc4)c(CN4CCOCC4)n3c2)cc1 ; c1ccc(-c2ccc3nc(-c4ccc(-c5ncon5)cc4)c(CN4CCOCC4)n3c2)cc1 ; CCOC(=O)c1nc2ccc(-c3ccccc3)cn2c1CN1CCOCC1 ; Clc1ccc(-c2nc3ccc(-c4ccccc4)cn3c2CN2CCN(c3ccccc3)CC2)cc1 ; Clc1ccc(-c2nc3ccc(-c4ccccc4)cn3c2CN2CCOC(c3ccccc3)C2)cc1]

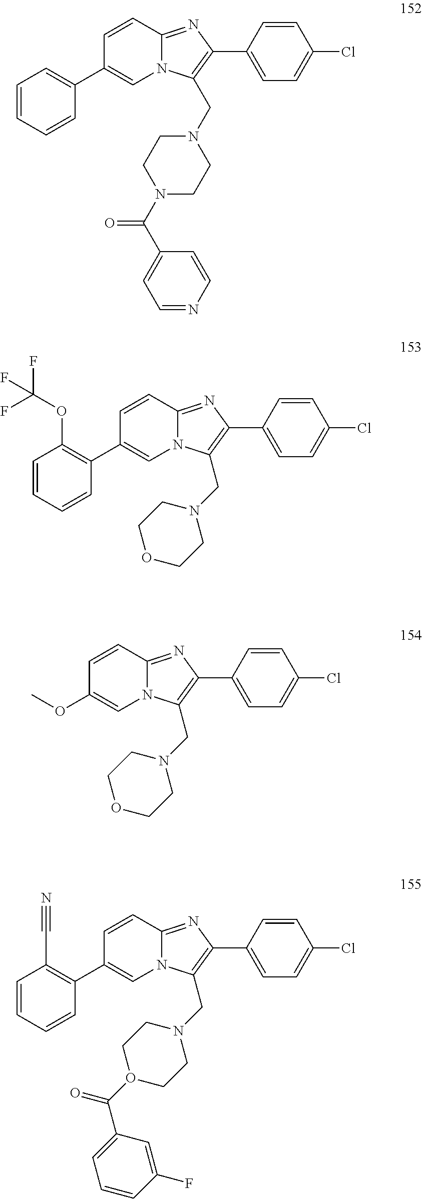

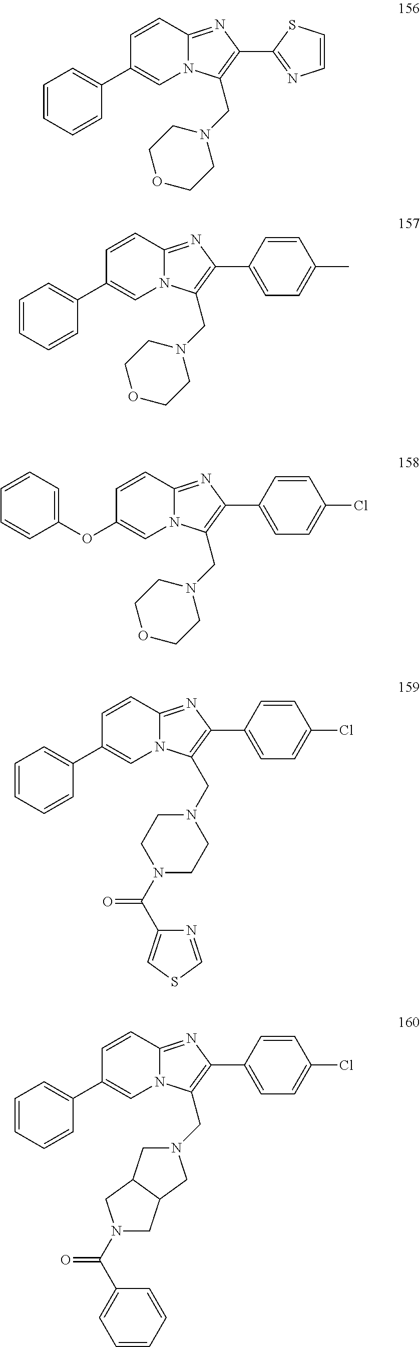 [SMILES: c1ccc(-c2ccc3nc(-c4nccs4)c(CN4CCOCC4)n3c2)cc1 ; Cc1ccc(-c2nc3ccc(-c4ccccc4)cn3c2CN2CCOCC2)cc1 ; Clc1ccc(-c2nc3ccc(Oc4ccccc4)cn3c2CN2CCOCC2)cc1 ; O=C(c1cscn1)N1CCN(Cc2c(-c3ccc(Cl)cc3)nc3ccc(-c4ccccc4)cn23)CC1 ; O=C(c1ccccc1)N1CC2CN(Cc3c(-c4ccc(Cl)cc4)nc4ccc(-c5ccccc5)cn34)CC2C1]

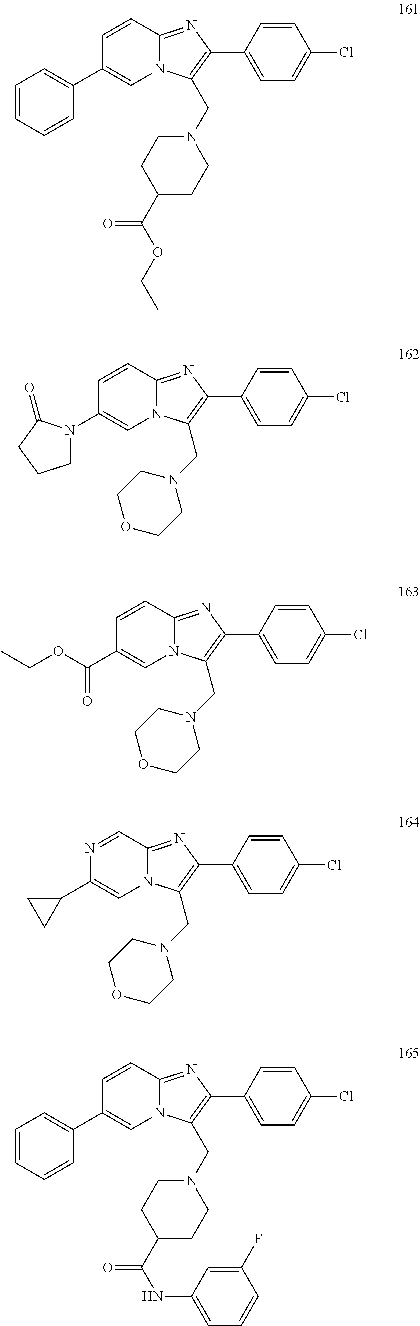 [SMILES: CCOC(=O)C1CCN(Cc2c(-c3ccc(Cl)cc3)nc3ccc(-c4ccccc4)cn23)CC1 ; O=C1CCCN1c1ccc2nc(-c3ccc(Cl)cc3)c(CN3CCOCC3)n2c1 ; CCOC(=O)c1ccc2nc(-c3ccc(Cl)cc3)c(CN3CCOCC3)n2c1 ; Clc1ccc(-c2nc3cnc(C4CC4)cn3c2CN2CCOCC2)cc1 ; O=C(Nc1cccc(F)c1)C1CCN(Cc2c(-c3ccc(Cl)cc3)nc3ccc(-c4ccccc4)cn23)CC1]

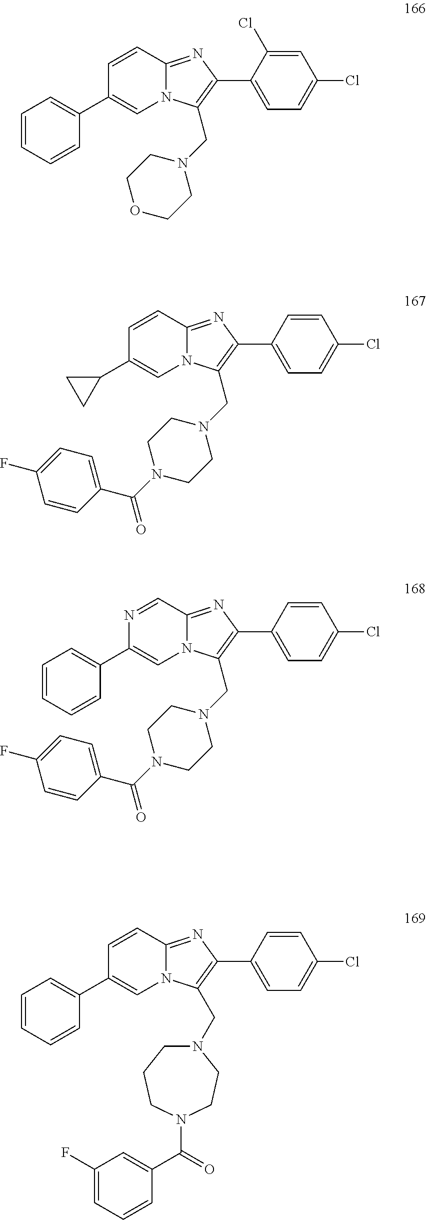 [SMILES: Clc1ccc(-c2nc3ccc(-c4ccccc4)cn3c2CN2CCOCC2)c(Cl)c1 ; O=C(c1ccc(F)cc1)N1CCN(Cc2c(-c3ccc(Cl)cc3)nc3ccc(C4CC4)cn23)CC1 ; O=C(c1ccc(F)cc1)N1CCN(Cc2c(-c3ccc(Cl)cc3)nc3cnc(-c4ccccc4)cn23)CC1 ; O=C(c1cccc(F)c1)N1CCCN(Cc2c(-c3ccc(Cl)cc3)nc3ccc(-c4ccccc4)cn23)CC1]

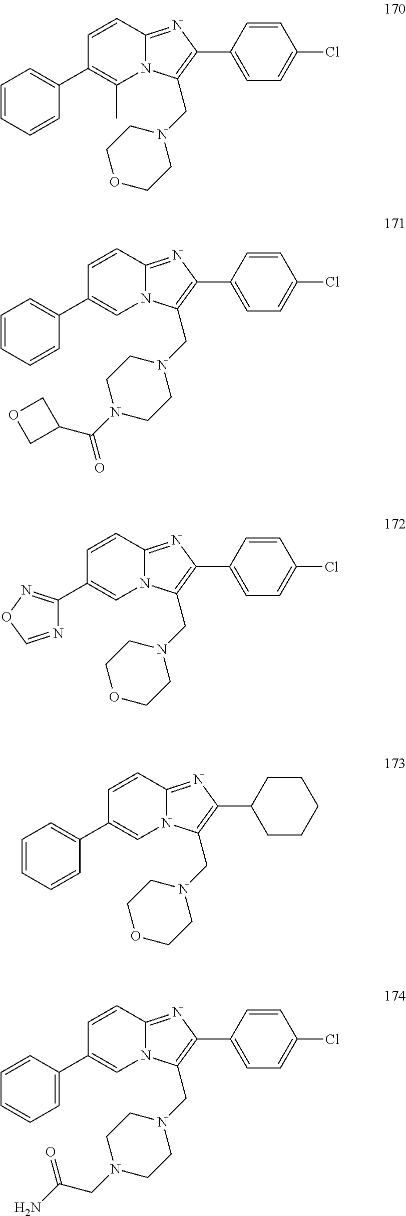 [SMILES: Cc1c(-c2ccccc2)ccc2nc(-c3ccc(Cl)cc3)c(CN3CCOCC3)n12 ; O=C(C1COC1)N1CCN(Cc2c(-c3ccc(Cl)cc3)nc3ccc(-c4ccccc4)cn23)CC1 ; Clc1ccc(-c2nc3ccc(-c4ncon4)cn3c2CN2CCOCC2)cc1 ; c1ccc(-c2ccc3nc(C4CCCCC4)c(CN4CCOCC4)n3c2)cc1 ; NC(=O)CN1CCN(Cc2c(-c3ccc(Cl)cc3)nc3ccc(-c4ccccc4)cn23)CC1]

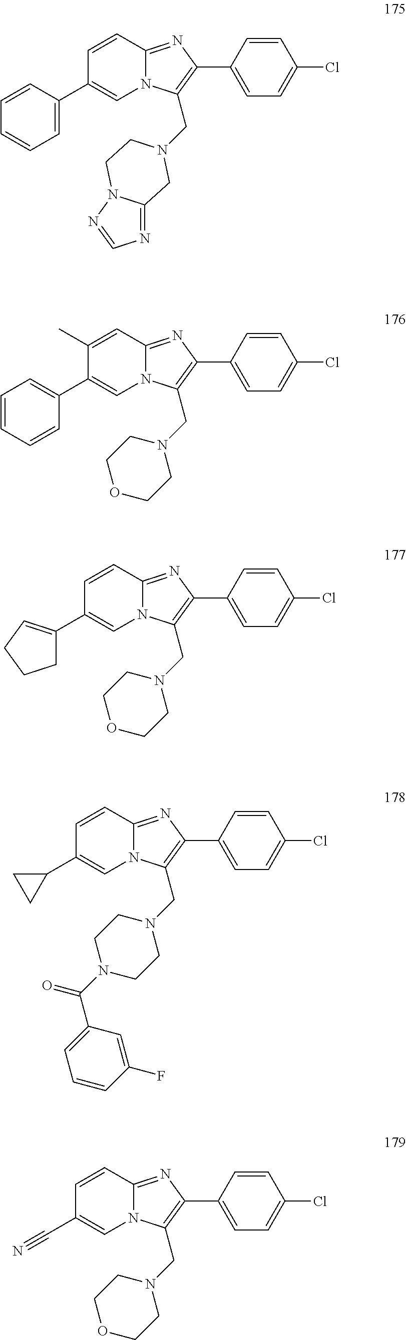 [SMILES: Clc1ccc(-c2nc3ccc(-c4ccccc4)cn3c2CN2CCn3ncnc3C2)cc1 ; Cc1cc2nc(-c3ccc(Cl)cc3)c(CN3CCOCC3)n2cc1-c1ccccc1 ; Clc1ccc(-c2nc3ccc(C4=CCCC4)cn3c2CN2CCOCC2)cc1 ; O=C(c1cccc(F)c1)N1CCN(Cc2c(-c3ccc(Cl)cc3)nc3ccc(C4CC4)cn23)CC1 ; N#Cc1ccc2nc(-c3ccc(Cl)cc3)c(CN3CCOCC3)n2c1]

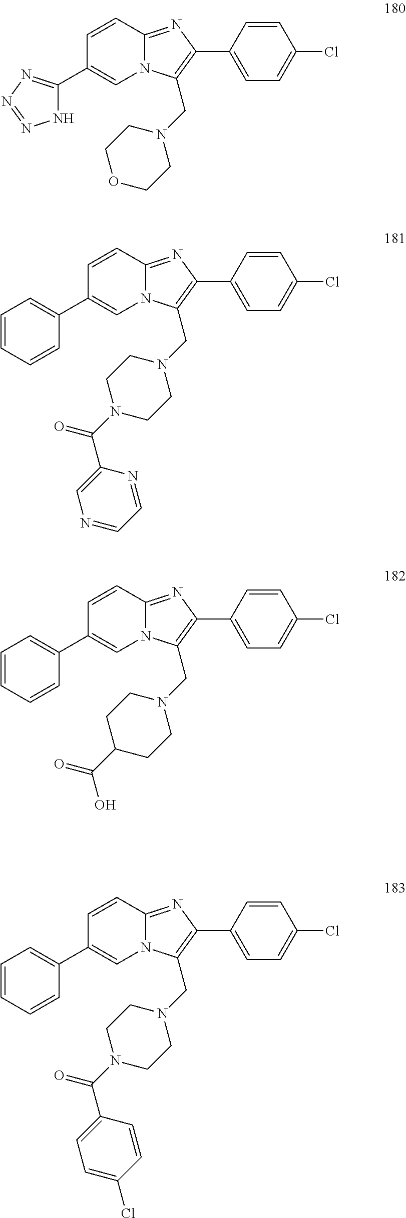 [SMILES: Clc1ccc(-c2nc3ccc(-c4nnn[nH]4)cn3c2CN2CCOCC2)cc1 ; O=C(c1cnccn1)N1CCN(Cc2c(-c3ccc(Cl)cc3)nc3ccc(-c4ccccc4)cn23)CC1 ; O=C(O)C1CCN(Cc2c(-c3ccc(Cl)cc3)nc3ccc(-c4ccccc4)cn23)CC1 ; O=C(c1ccc(Cl)cc1)N1CCN(Cc2c(-c3ccc(Cl)cc3)nc3ccc(-c4ccccc4)cn23)CC1]

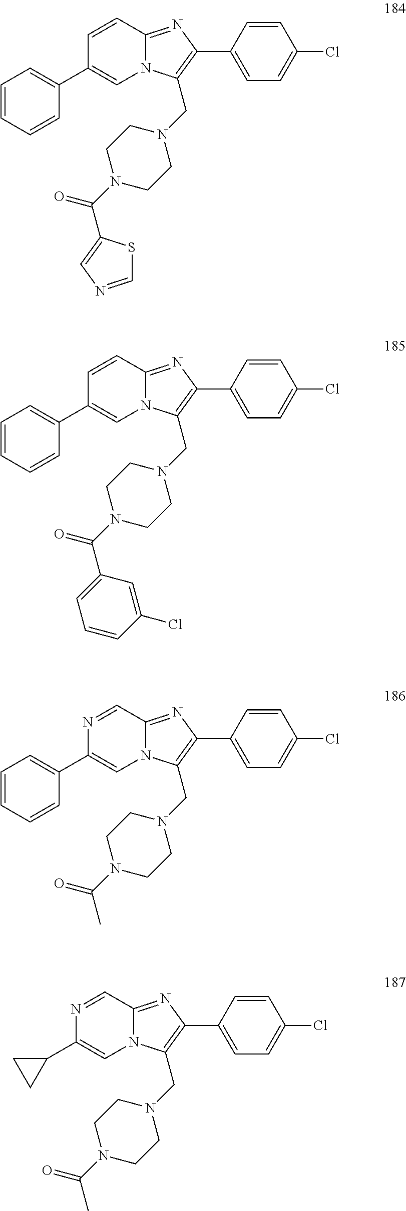 [SMILES: O=C(c1cncs1)N1CCN(Cc2c(-c3ccc(Cl)cc3)nc3ccc(-c4ccccc4)cn23)CC1 ; O=C(c1cccc(Cl)c1)N1CCN(Cc2c(-c3ccc(Cl)cc3)nc3ccc(-c4ccccc4)cn23)CC1 ; CC(=O)N1CCN(Cc2c(-c3ccc(Cl)cc3)nc3cnc(-c4ccccc4)cn23)CC1 ; CC(=O)N1CCN(Cc2c(-c3ccc(Cl)cc3)nc3cnc(C4CC4)cn23)CC1]

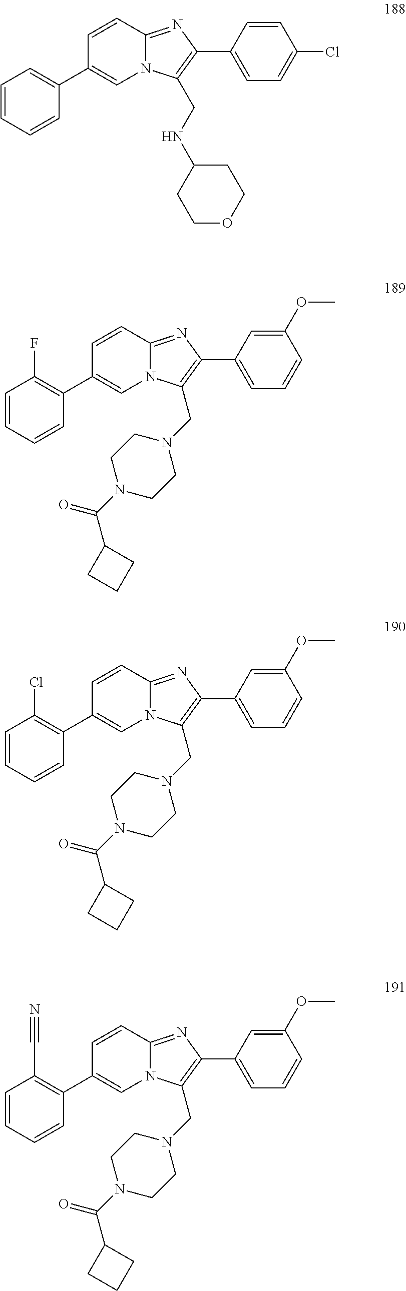 [SMILES: Clc1ccc(-c2nc3ccc(-c4ccccc4)cn3c2CNC2CCOCC2)cc1 ; COc1cccc(-c2nc3ccc(-c4ccccc4F)cn3c2CN2CCN(C(=O)C3CCC3)CC2)c1 ; COc1cccc(-c2nc3ccc(-c4ccccc4Cl)cn3c2CN2CCN(C(=O)C3CCC3)CC2)c1 ; COc1cccc(-c2nc3ccc(-c4ccccc4C#N)cn3c2CN2CCN(C(=O)C3CCC3)CC2)c1]

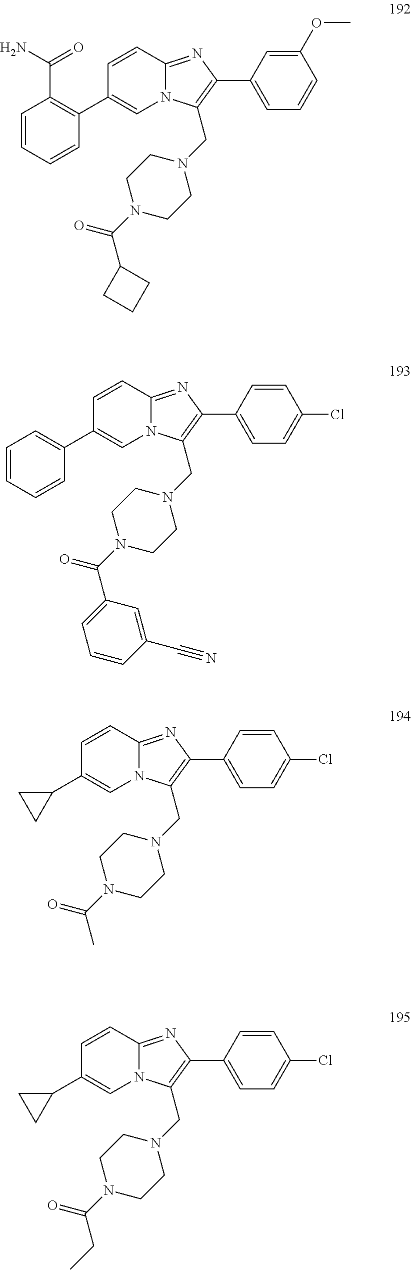 [SMILES: COc1cccc(-c2nc3ccc(-c4ccccc4C(N)=O)cn3c2CN2CCN(C(=O)C3CCC3)CC2)c1 ; N#Cc1cccc(C(=O)N2CCN(Cc3c(-c4ccc(Cl)cc4)nc4ccc(-c5ccccc5)cn34)CC2)c1 ; CC(=O)N1CCN(Cc2c(-c3ccc(Cl)cc3)nc3ccc(C4CC4)cn23)CC1 ; CCC(=O)N1CCN(Cc2c(-c3ccc(Cl)cc3)nc3ccc(C4CC4)cn23)CC1]

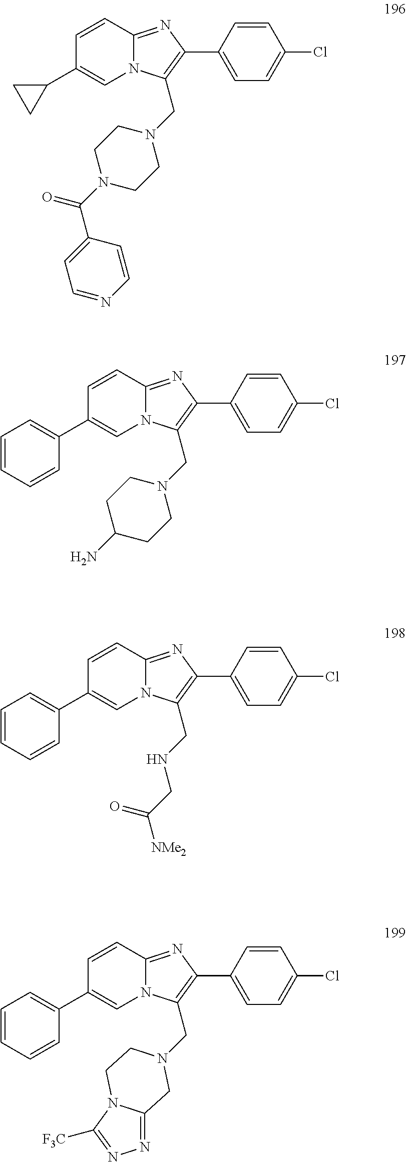 [SMILES: O=C(c1ccncc1)N1CCN(Cc2c(-c3ccc(Cl)cc3)nc3ccc(C4CC4)cn23)CC1 ; NC1CCN(Cc2c(-c3ccc(Cl)cc3)nc3ccc(-c4ccccc4)cn23)CC1 ; CN(C)C(=O)CNCc1c(-c2ccc(Cl)cc2)nc2ccc(-c3ccccc3)cn12 ; FC(F)(F)c1nnc2n1CCN(Cc1c(-c3ccc(Cl)cc3)nc3ccc(-c4ccccc4)cn13)C2]

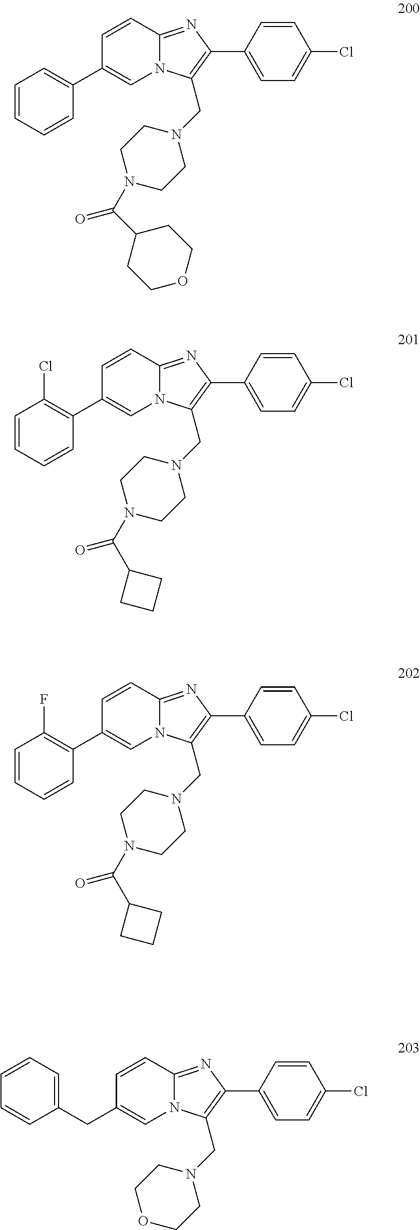 [SMILES: O=C(C1CCOCC1)N1CCN(Cc2c(-c3ccc(Cl)cc3)nc3ccc(-c4ccccc4)cn23)CC1 ; O=C(C1CCC1)N1CCN(Cc2c(-c3ccc(Cl)cc3)nc3ccc(-c4ccccc4Cl)cn23)CC1 ; O=C(C1CCC1)N1CCN(Cc2c(-c3ccc(Cl)cc3)nc3ccc(-c4ccccc4F)cn23)CC1 ; Clc1ccc(-c2nc3ccc(Cc4ccccc4)cn3c2CN2CCOCC2)cc1]

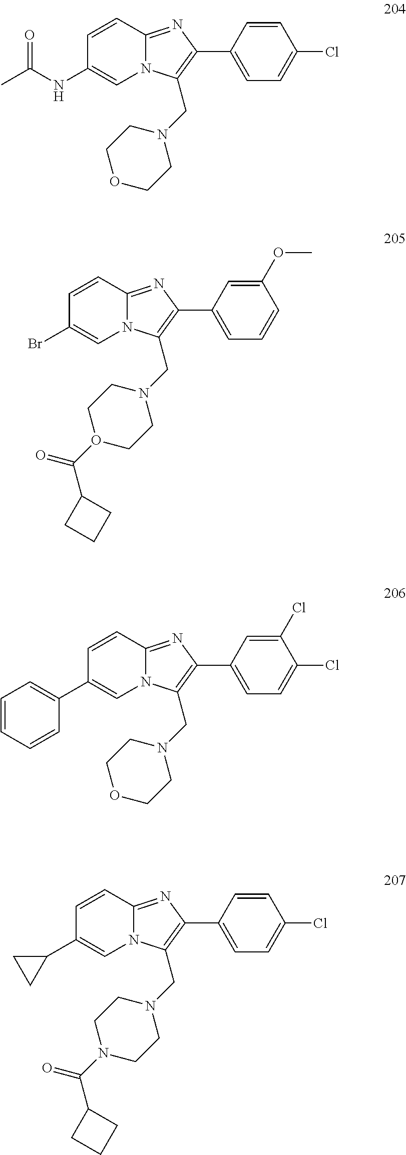

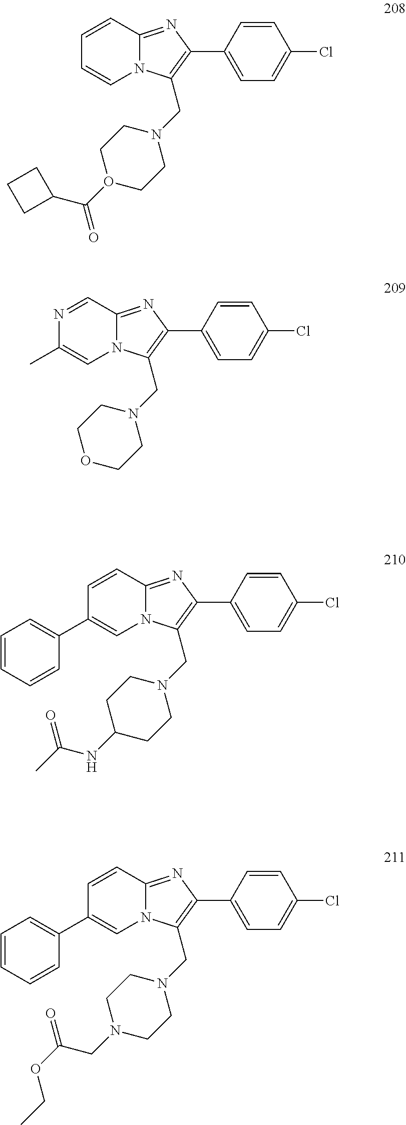

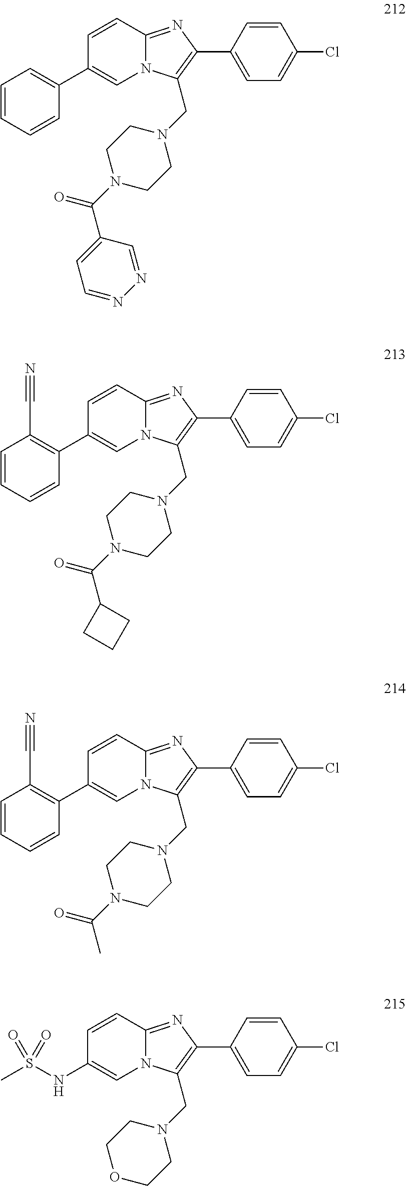 [SMILES: O=C(c1ccnnc1)N1CCN(Cc2c(-c3ccc(Cl)cc3)nc3ccc(-c4ccccc4)cn23)CC1 ; N#Cc1ccccc1-c1ccc2nc(-c3ccc(Cl)cc3)c(CN3CCN(C(=O)C4CCC4)CC3)n2c1 ; CC(=O)N1CCN(Cc2c(-c3ccc(Cl)cc3)nc3ccc(-c4ccccc4C#N)cn23)CC1 ; CS(=O)(=O)Nc1ccc2nc(-c3ccc(Cl)cc3)c(CN3CCOCC3)n2c1]

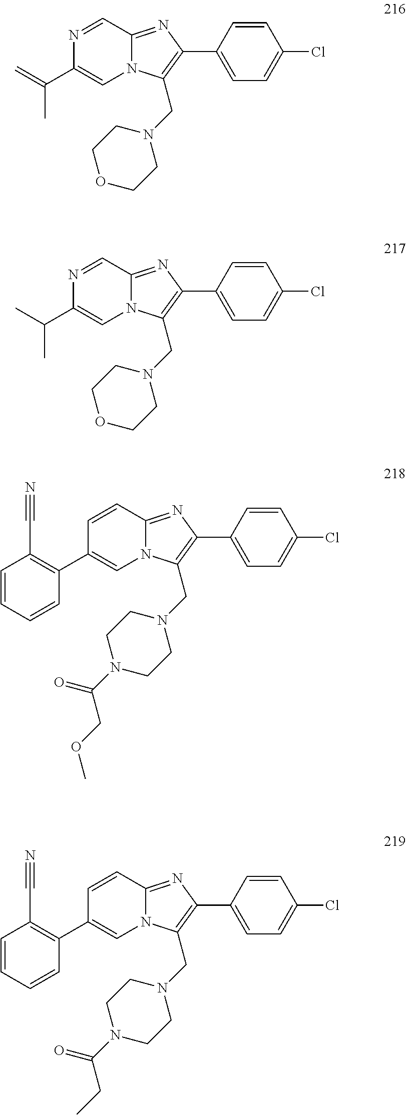 [SMILES: C=C(C)c1cn2c(CN3CCOCC3)c(-c3ccc(Cl)cc3)nc2cn1 ; CC(C)c1cn2c(CN3CCOCC3)c(-c3ccc(Cl)cc3)nc2cn1 ; COCC(=O)N1CCN(Cc2c(-c3ccc(Cl)cc3)nc3ccc(-c4ccccc4C#N)cn23)CC1 ; CCC(=O)N1CCN(Cc2c(-c3ccc(Cl)cc3)nc3ccc(-c4ccccc4C#N)cn23)CC1]

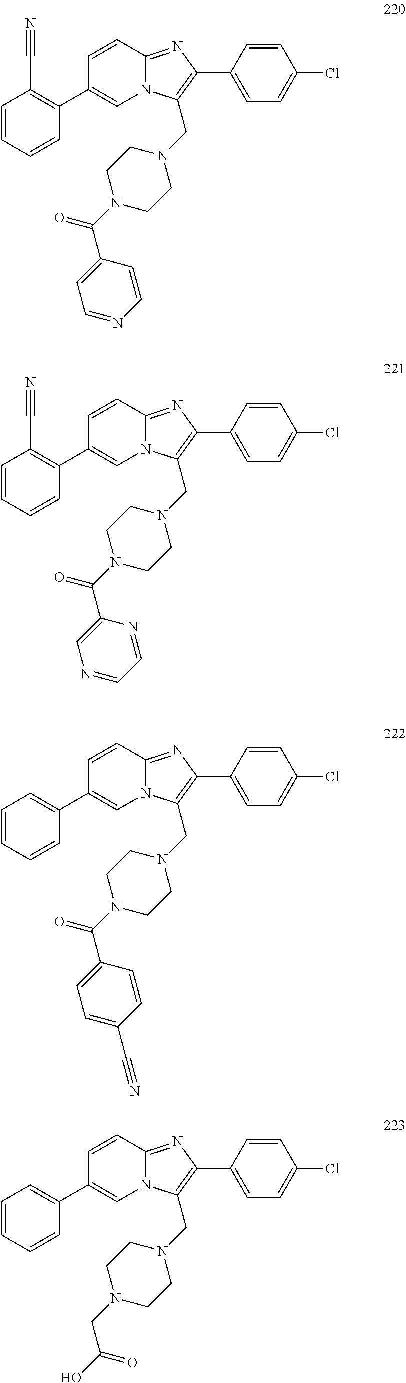 [SMILES: N#Cc1ccccc1-c1ccc2nc(-c3ccc(Cl)cc3)c(CN3CCN(C(=O)c4ccncc4)CC3)n2c1 ; N#Cc1ccccc1-c1ccc2nc(-c3ccc(Cl)cc3)c(CN3CCN(C(=O)c4cnccn4)CC3)n2c1 ; N#Cc1ccc(C(=O)N2CCN(Cc3c(-c4ccc(Cl)cc4)nc4ccc(-c5ccccc5)cn34)CC2)cc1 ; O=C(O)CN1CCN(Cc2c(-c3ccc(Cl)cc3)nc3ccc(-c4ccccc4)cn23)CC1]

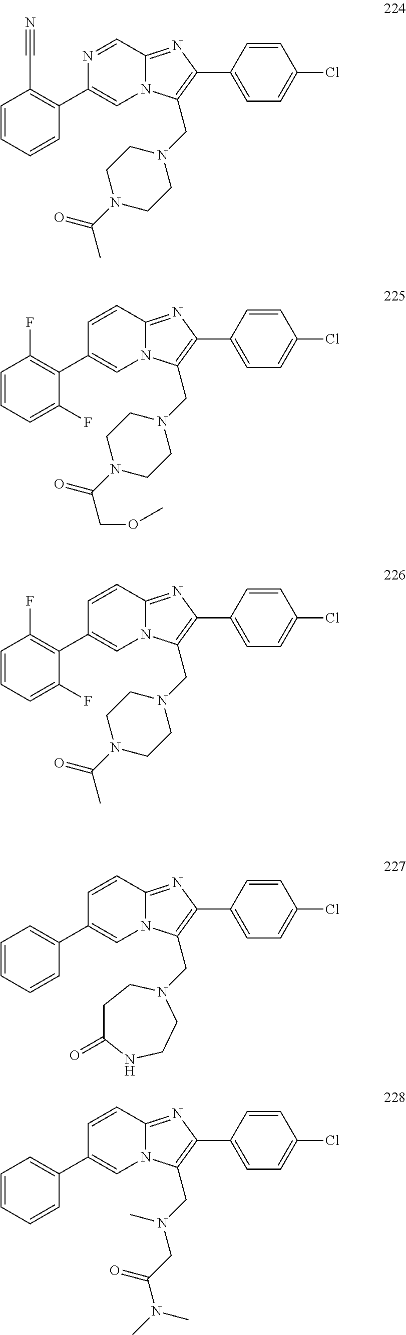 [SMILES: CC(=O)N1CCN(Cc2c(-c3ccc(Cl)cc3)nc3cnc(-c4ccccc4C#N)cn23)CC1 ; COCC(=O)N1CCN(Cc2c(-c3ccc(Cl)cc3)nc3ccc(-c4c(F)cccc4F)cn23)CC1 ; CC(=O)N1CCN(Cc2c(-c3ccc(Cl)cc3)nc3ccc(-c4c(F)cccc4F)cn23)CC1 ; O=C1CCN(Cc2c(-c3ccc(Cl)cc3)nc3ccc(-c4ccccc4)cn23)CCN1 ; CN(CC(=O)N(C)C)Cc1c(-c2ccc(Cl)cc2)nc2ccc(-c3ccccc3)cn12]

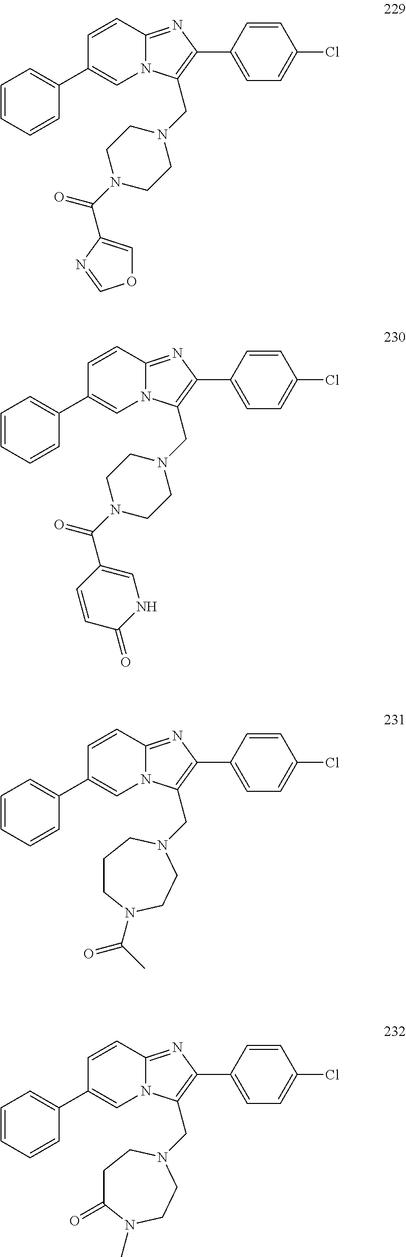 [SMILES: O=C(c1cocn1)N1CCN(Cc2c(-c3ccc(Cl)cc3)nc3ccc(-c4ccccc4)cn23)CC1 ; O=C(c1ccc(=O)[nH]c1)N1CCN(Cc2c(-c3ccc(Cl)cc3)nc3ccc(-c4ccccc4)cn23)CC1 ; CC(=O)N1CCCN(Cc2c(-c3ccc(Cl)cc3)nc3ccc(-c4ccccc4)cn23)CC1 ; CN1CCN(Cc2c(-c3ccc(Cl)cc3)nc3ccc(-c4ccccc4)cn23)CCC1=O]

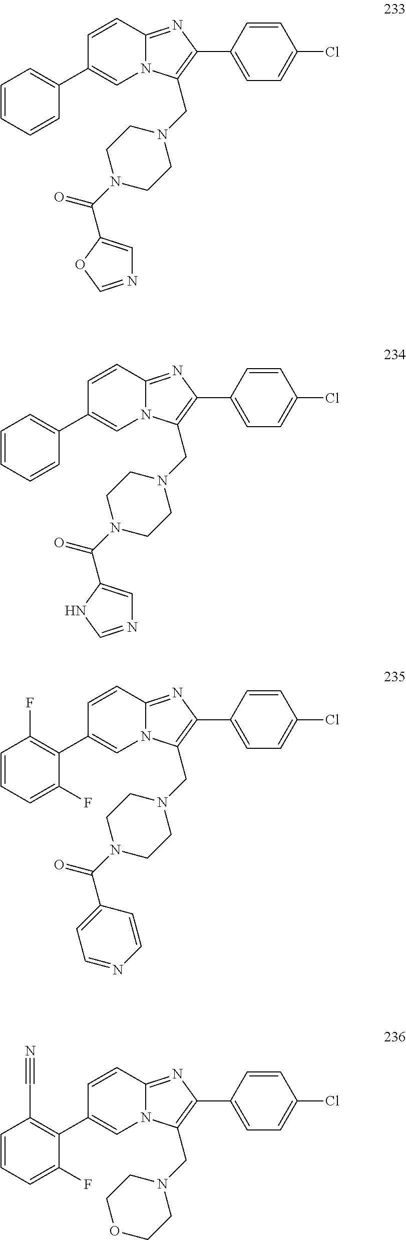 [SMILES: O=C(c1cnco1)N1CCN(Cc2c(-c3ccc(Cl)cc3)nc3ccc(-c4ccccc4)cn23)CC1 ; O=C(c1cnc[nH]1)N1CCN(Cc2c(-c3ccc(Cl)cc3)nc3ccc(-c4ccccc4)cn23)CC1 ; O=C(c1ccncc1)N1CCN(Cc2c(-c3ccc(Cl)cc3)nc3ccc(-c4c(F)cccc4F)cn23)CC1 ; N#Cc1cccc(F)c1-c1ccc2nc(-c3ccc(Cl)cc3)c(CN3CCOCC3)n2c1]

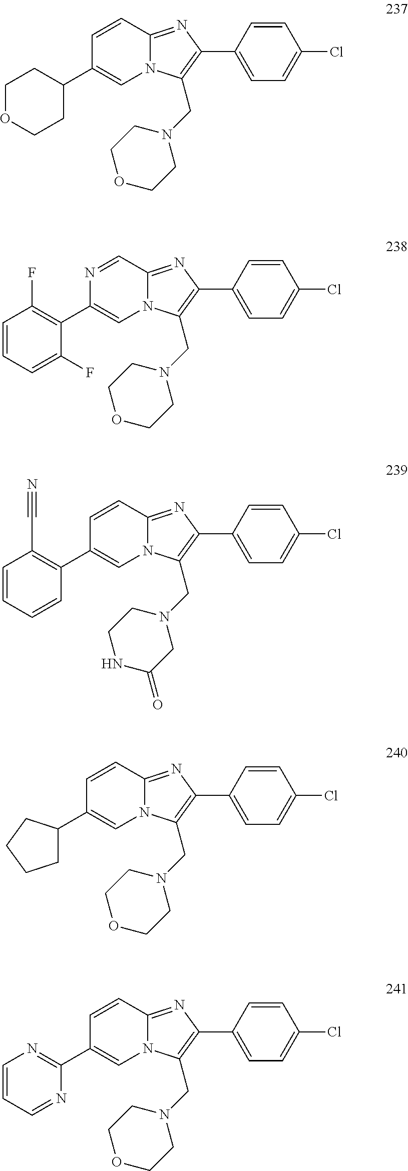 [SMILES: Clc1ccc(-c2nc3ccc(C4CCOCC4)cn3c2CN2CCOCC2)cc1 ; Fc1cccc(F)c1-c1cn2c(CN3CCOCC3)c(-c3ccc(Cl)cc3)nc2cn1 ; N#Cc1ccccc1-c1ccc2nc(-c3ccc(Cl)cc3)c(CN3CCNC(=O)C3)n2c1 ; Clc1ccc(-c2nc3ccc(C4CCCC4)cn3c2CN2CCOCC2)cc1 ; Clc1ccc(-c2nc3ccc(-c4ncccn4)cn3c2CN2CCOCC2)cc1]

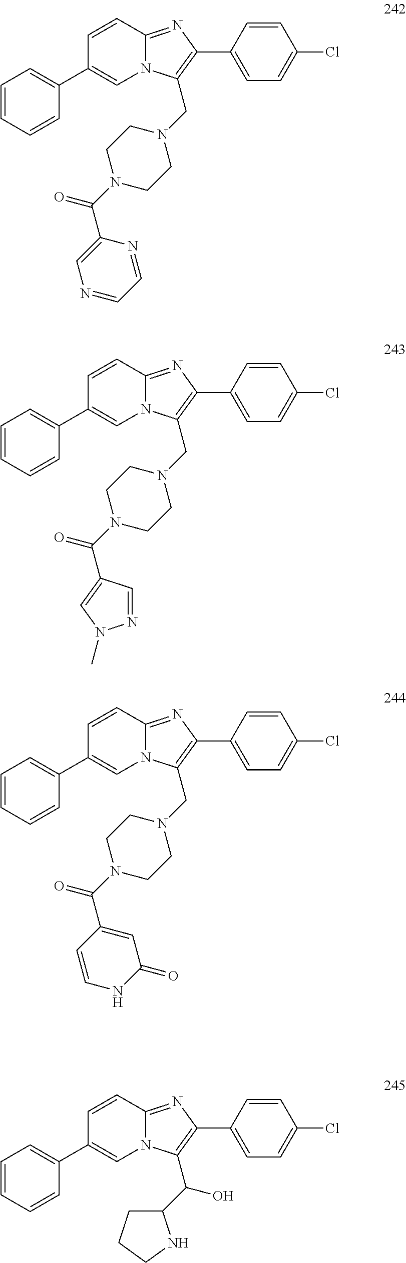 [SMILES: O=C(c1cnccn1)N1CCN(Cc2c(-c3ccc(Cl)cc3)nc3ccc(-c4ccccc4)cn23)CC1 ; Cn1cc(C(=O)N2CCN(Cc3c(-c4ccc(Cl)cc4)nc4ccc(-c5ccccc5)cn34)CC2)cn1 ; O=C(c1cc[nH]c(=O)c1)N1CCN(Cc2c(-c3ccc(Cl)cc3)nc3ccc(-c4ccccc4)cn23)CC1 ; OC(c1c(-c2ccc(Cl)cc2)nc2ccc(-c3ccccc3)cn12)C1CCCN1]

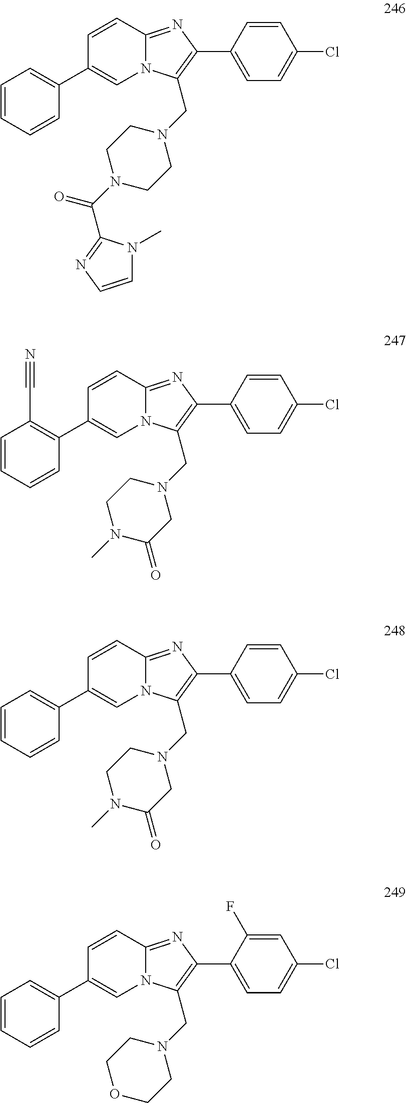 [SMILES: Cn1ccnc1C(=O)N1CCN(Cc2c(-c3ccc(Cl)cc3)nc3ccc(-c4ccccc4)cn23)CC1 ; CN1CCN(Cc2c(-c3ccc(Cl)cc3)nc3ccc(-c4ccccc4C#N)cn23)CC1=O ; CN1CCN(Cc2c(-c3ccc(Cl)cc3)nc3ccc(-c4ccccc4)cn23)CC1=O ; Fc1cc(Cl)ccc1-c1nc2ccc(-c3ccccc3)cn2c1CN1CCOCC1]

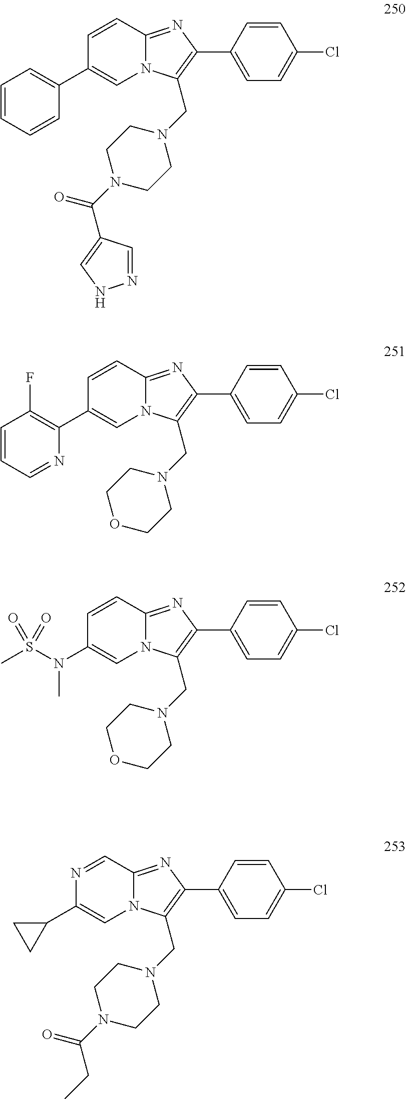 [SMILES: O=C(c1cn[nH]c1)N1CCN(Cc2c(-c3ccc(Cl)cc3)nc3ccc(-c4ccccc4)cn23)CC1 ; Fc1cccnc1-c1ccc2nc(-c3ccc(Cl)cc3)c(CN3CCOCC3)n2c1 ; CN(c1ccc2nc(-c3ccc(Cl)cc3)c(CN3CCOCC3)n2c1)S(C)(=O)=O ; CCC(=O)N1CCN(Cc2c(-c3ccc(Cl)cc3)nc3cnc(C4CC4)cn23)CC1]

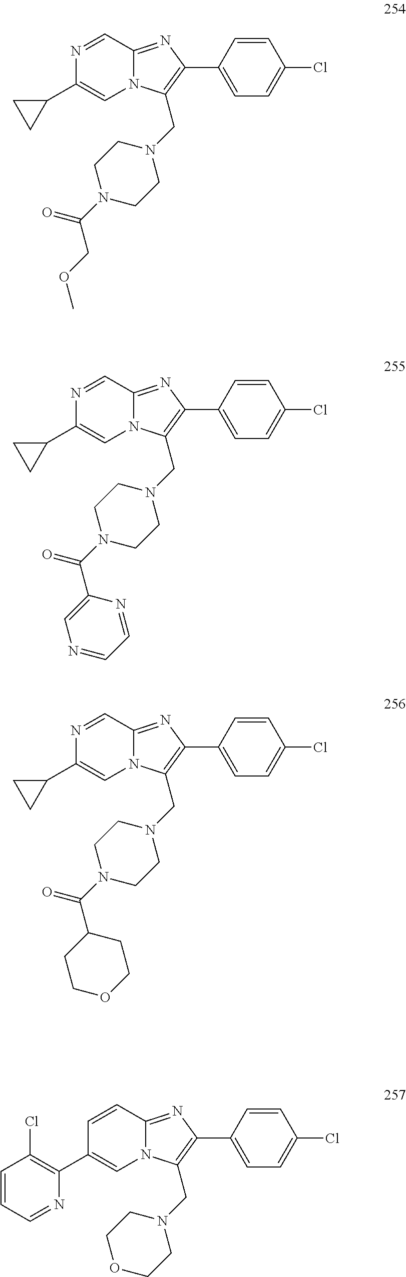 [SMILES: COCC(=O)N1CCN(Cc2c(-c3ccc(Cl)cc3)nc3cnc(C4CC4)cn23)CC1 ; O=C(c1cnccn1)N1CCN(Cc2c(-c3ccc(Cl)cc3)nc3cnc(C4CC4)cn23)CC1 ; O=C(C1CCOCC1)N1CCN(Cc2c(-c3ccc(Cl)cc3)nc3cnc(C4CC4)cn23)CC1 ; Clc1ccc(-c2nc3ccc(-c4ncccc4Cl)cn3c2CN2CCOCC2)cc1]

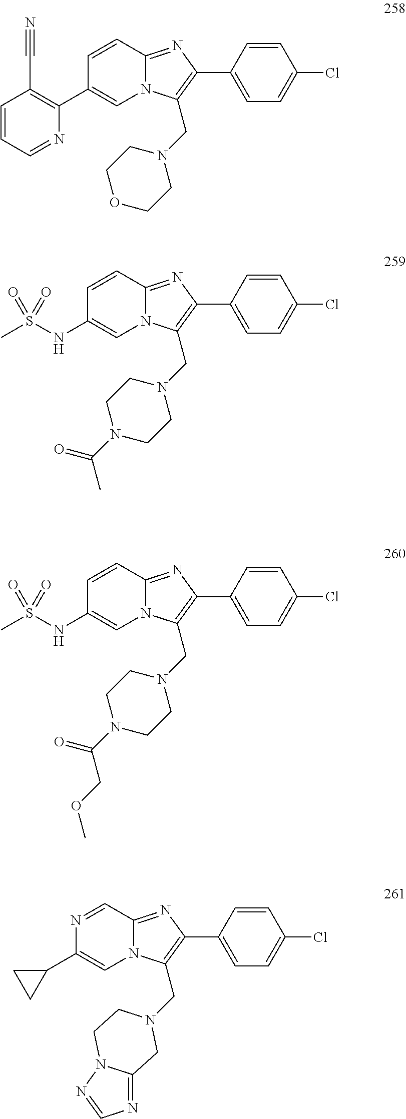 [SMILES: N#Cc1cccnc1-c1ccc2nc(-c3ccc(Cl)cc3)c(CN3CCOCC3)n2c1 ; CC(=O)N1CCN(Cc2c(-c3ccc(Cl)cc3)nc3ccc(NS(C)(=O)=O)cn23)CC1 ; COCC(=O)N1CCN(Cc2c(-c3ccc(Cl)cc3)nc3ccc(NS(C)(=O)=O)cn23)CC1 ; Clc1ccc(-c2nc3cnc(C4CC4)cn3c2CN2CCn3ncnc3C2)cc1]

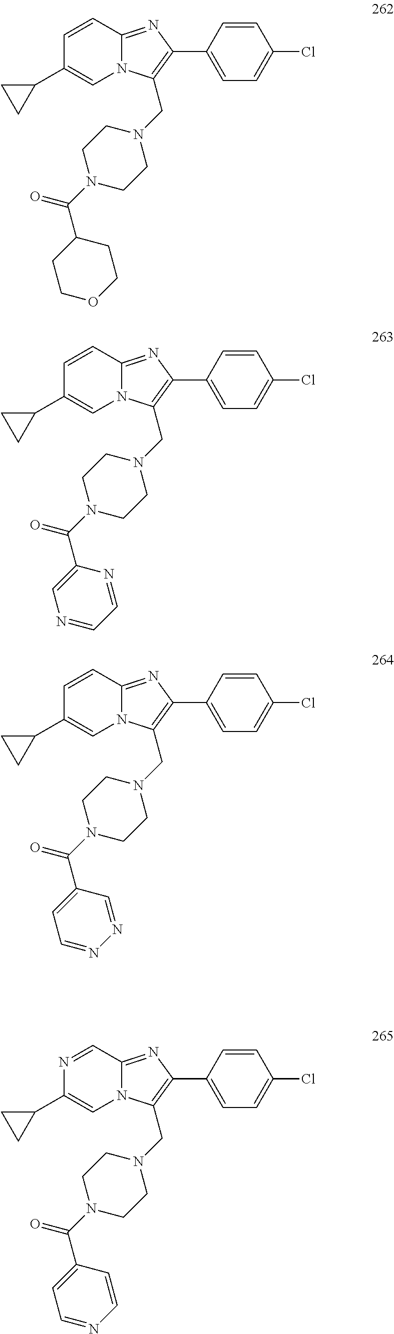

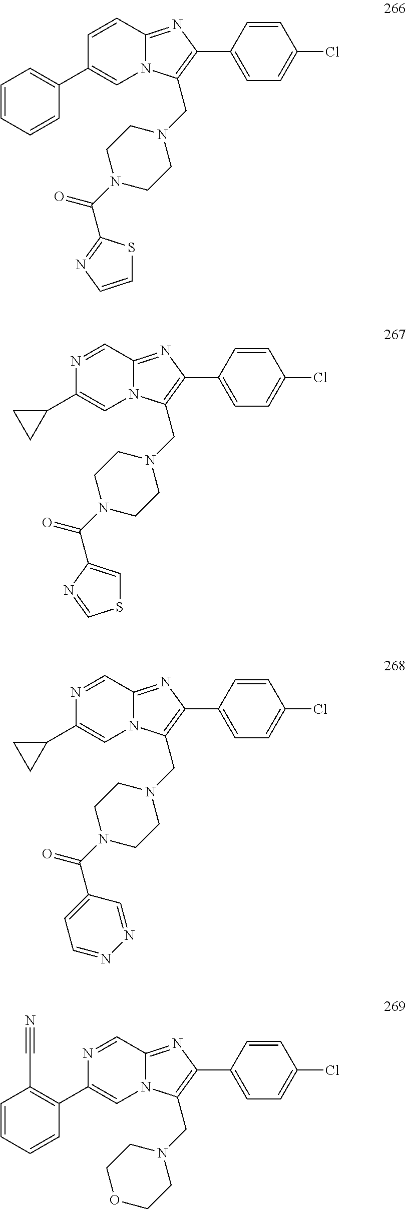 [SMILES: O=C(c1nccs1)N1CCN(Cc2c(-c3ccc(Cl)cc3)nc3ccc(-c4ccccc4)cn23)CC1 ; O=C(c1cscn1)N1CCN(Cc2c(-c3ccc(Cl)cc3)nc3cnc(C4CC4)cn23)CC1 ; O=C(c1ccnnc1)N1CCN(Cc2c(-c3ccc(Cl)cc3)nc3cnc(C4CC4)cn23)CC1 ; N#Cc1ccccc1-c1cn2c(CN3CCOCC3)c(-c3ccc(Cl)cc3)nc2cn1]

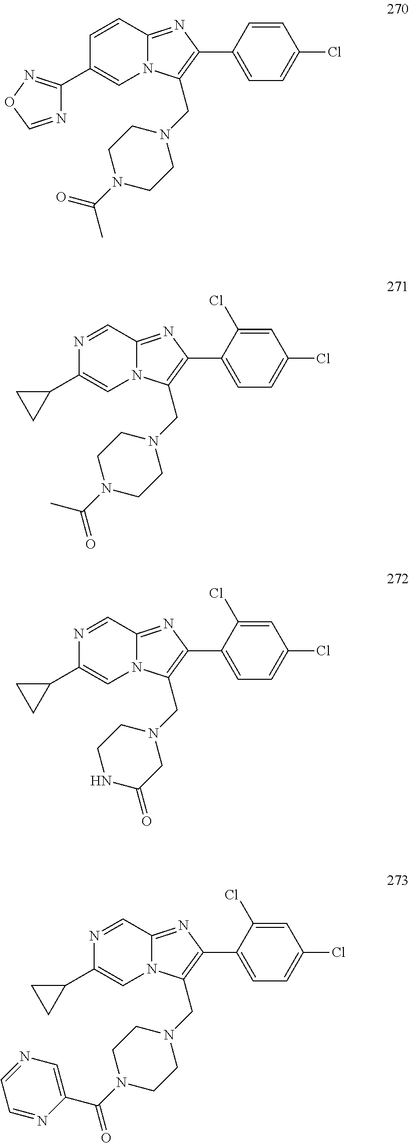 [SMILES: CC(=O)N1CCN(Cc2c(-c3ccc(Cl)cc3)nc3ccc(-c4ncon4)cn23)CC1 ; CC(=O)N1CCN(Cc2c(-c3ccc(Cl)cc3Cl)nc3cnc(C4CC4)cn23)CC1 ; O=C1CN(Cc2c(-c3ccc(Cl)cc3Cl)nc3cnc(C4CC4)cn23)CCN1 ; O=C(c1cnccn1)N1CCN(Cc2c(-c3ccc(Cl)cc3Cl)nc3cnc(C4CC4)cn23)CC1]

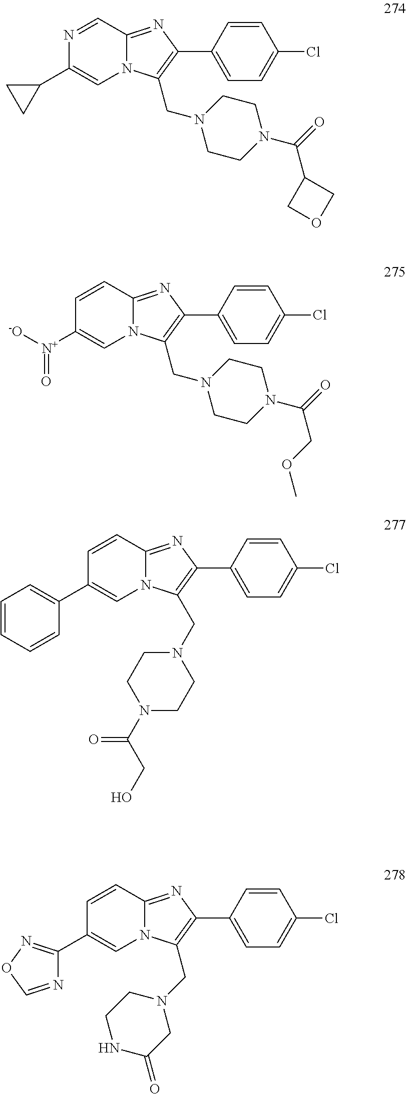

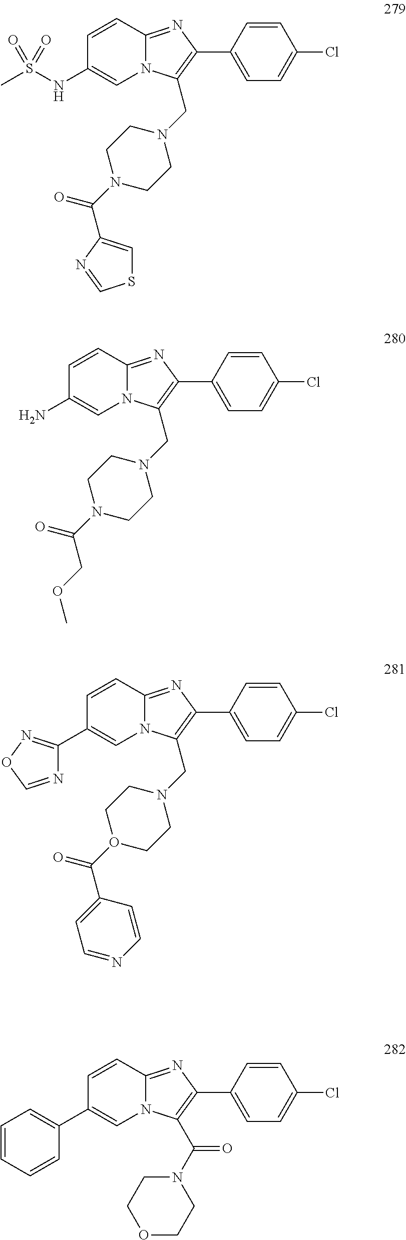

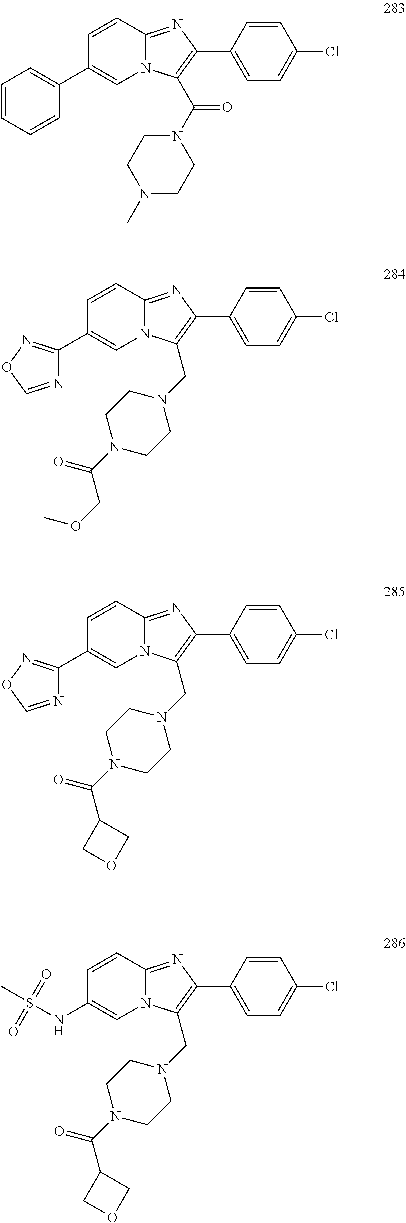 [SMILES: CN1CCN(C(=O)c2c(-c3ccc(Cl)cc3)nc3ccc(-c4ccccc4)cn23)CC1 ; COCC(=O)N1CCN(Cc2c(-c3ccc(Cl)cc3)nc3ccc(-c4ncon4)cn23)CC1 ; O=C(C1COC1)N1CCN(Cc2c(-c3ccc(Cl)cc3)nc3ccc(-c4ncon4)cn23)CC1 ; CS(=O)(=O)Nc1ccc2nc(-c3ccc(Cl)cc3)c(CN3CCN(C(=O)C4COC4)CC3)n2c1]

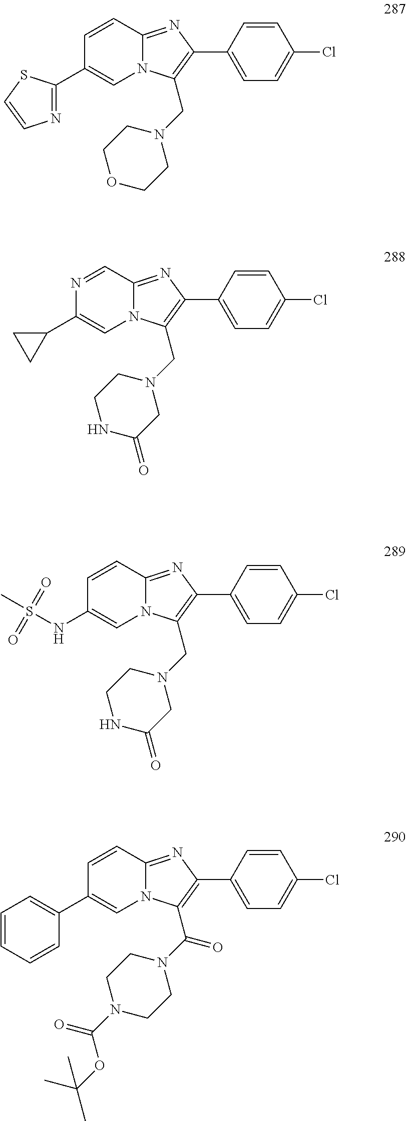 [SMILES: Clc1ccc(-c2nc3ccc(-c4nccs4)cn3c2CN2CCOCC2)cc1 ; O=C1CN(Cc2c(-c3ccc(Cl)cc3)nc3cnc(C4CC4)cn23)CCN1 ; CS(=O)(=O)Nc1ccc2nc(-c3ccc(Cl)cc3)c(CN3CCNC(=O)C3)n2c1 ; CC(C)(C)OC(=O)N1CCN(C(=O)c2c(-c3ccc(Cl)cc3)nc3ccc(-c4ccccc4)cn23)CC1]

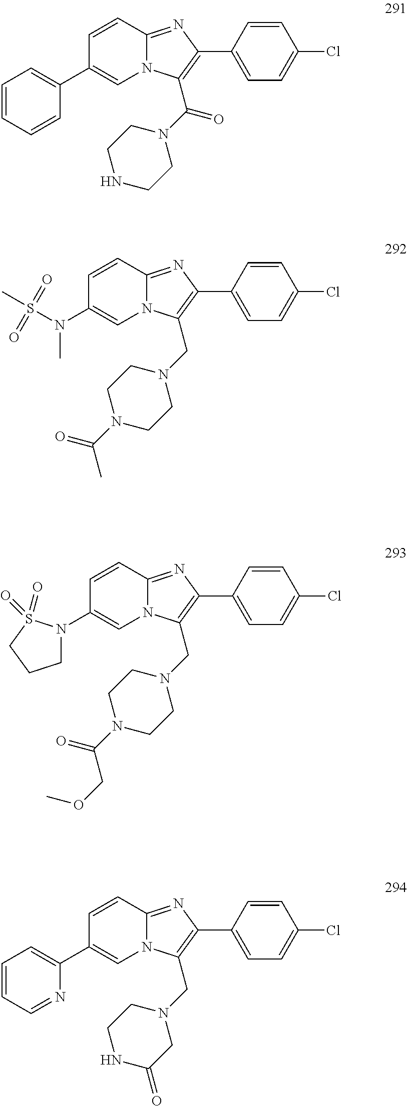 [SMILES: O=C(c1c(-c2ccc(Cl)cc2)nc2ccc(-c3ccccc3)cn12)N1CCNCC1 ; CC(=O)N1CCN(Cc2c(-c3ccc(Cl)cc3)nc3ccc(N(C)S(C)(=O)=O)cn23)CC1 ; COCC(=O)N1CCN(Cc2c(-c3ccc(Cl)cc3)nc3ccc(N4CCCS4(=O)=O)cn23)CC1 ; O=C1CN(Cc2c(-c3ccc(Cl)cc3)nc3ccc(-c4ccccn4)cn23)CCN1]

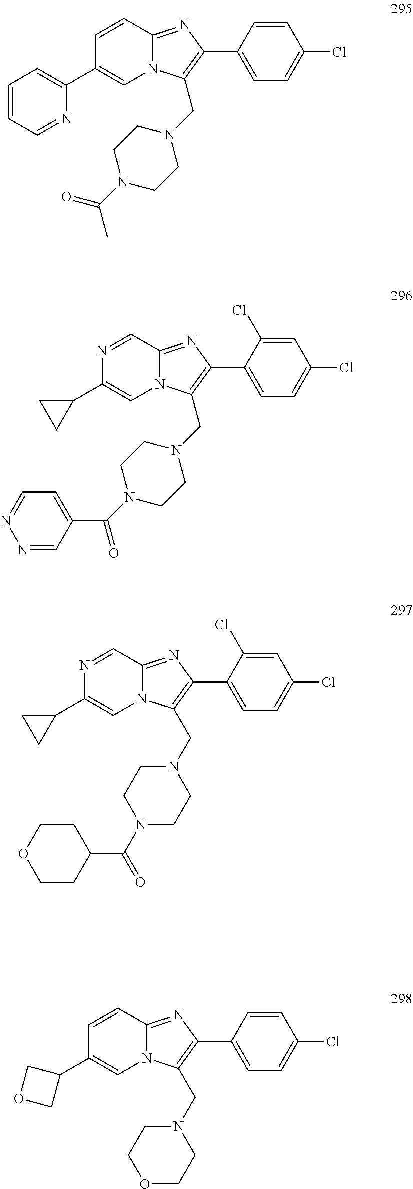 [SMILES: CC(=O)N1CCN(Cc2c(-c3ccc(Cl)cc3)nc3ccc(-c4ccccn4)cn23)CC1 ; O=C(c1ccnnc1)N1CCN(Cc2c(-c3ccc(Cl)cc3Cl)nc3cnc(C4CC4)cn23)CC1 ; O=C(C1CCOCC1)N1CCN(Cc2c(-c3ccc(Cl)cc3Cl)nc3cnc(C4CC4)cn23)CC1 ; Clc1ccc(-c2nc3ccc(C4COC4)cn3c2CN2CCOCC2)cc1]

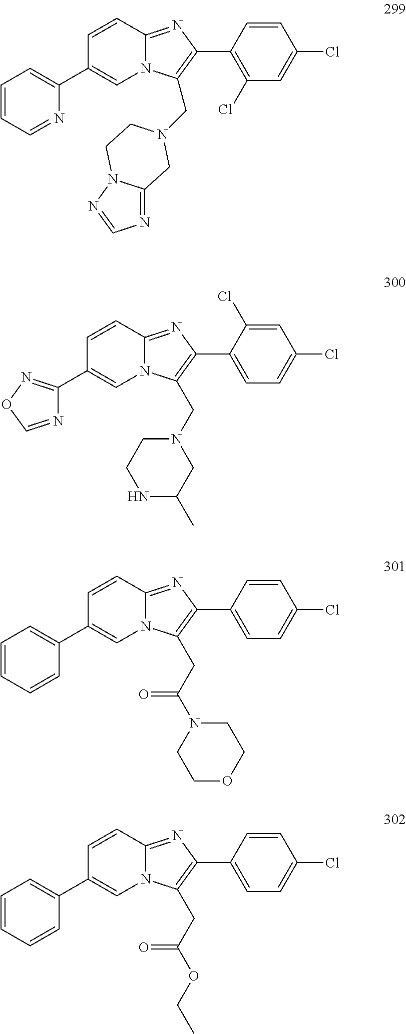 [SMILES: Clc1ccc(-c2nc3ccc(-c4ccccn4)cn3c2CN2CCn3ncnc3C2)c(Cl)c1 ; CC1CN(Cc2c(-c3ccc(Cl)cc3Cl)nc3ccc(-c4ncon4)cn23)CCN1 ; O=C(Cc1c(-c2ccc(Cl)cc2)nc2ccc(-c3ccccc3)cn12)N1CCOCC1 ; CCOC(=O)Cc1c(-c2ccc(Cl)cc2)nc2ccc(-c3ccccc3)cn12]

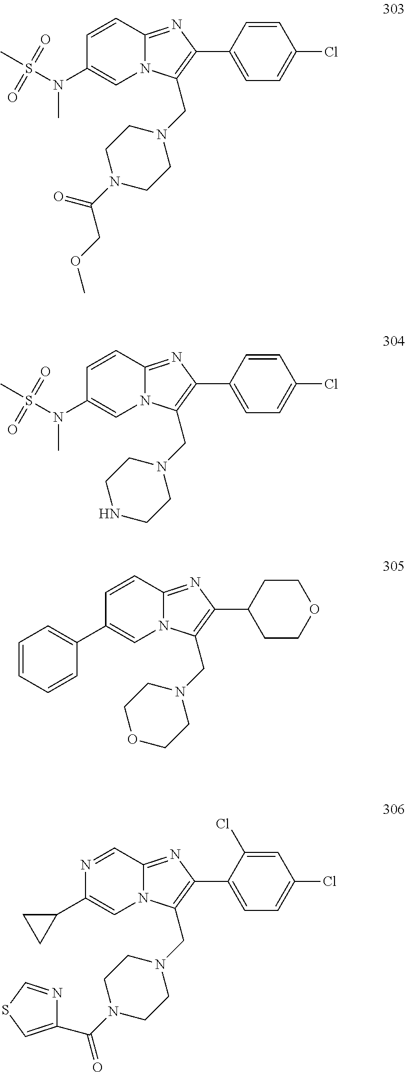 [SMILES: COCC(=O)N1CCN(Cc2c(-c3ccc(Cl)cc3)nc3ccc(N(C)S(C)(=O)=O)cn23)CC1 ; CN(c1ccc2nc(-c3ccc(Cl)cc3)c(CN3CCNCC3)n2c1)S(C)(=O)=O ; c1ccc(-c2ccc3nc(C4CCOCC4)c(CN4CCOCC4)n3c2)cc1 ; O=C(c1cscn1)N1CCN(Cc2c(-c3ccc(Cl)cc3Cl)nc3cnc(C4CC4)cn23)CC1]

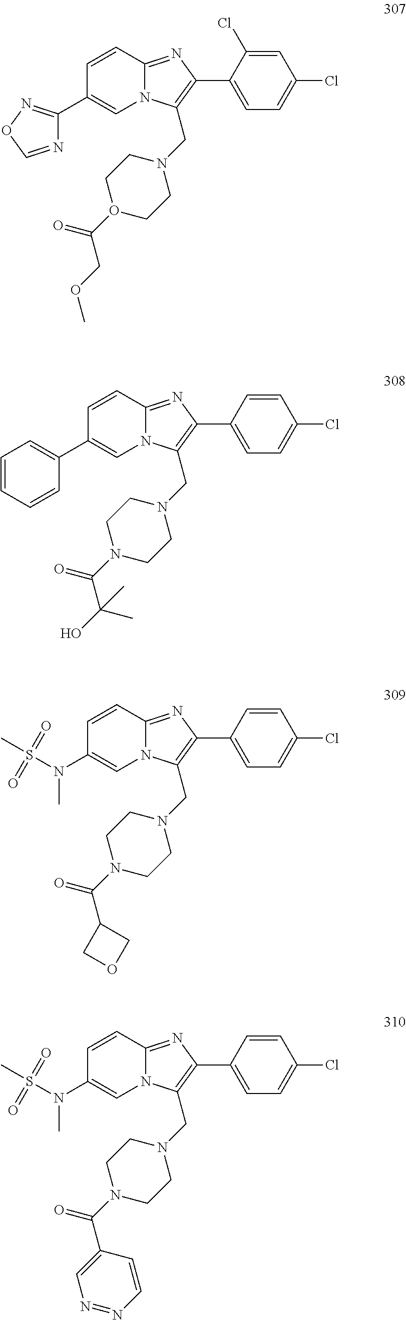

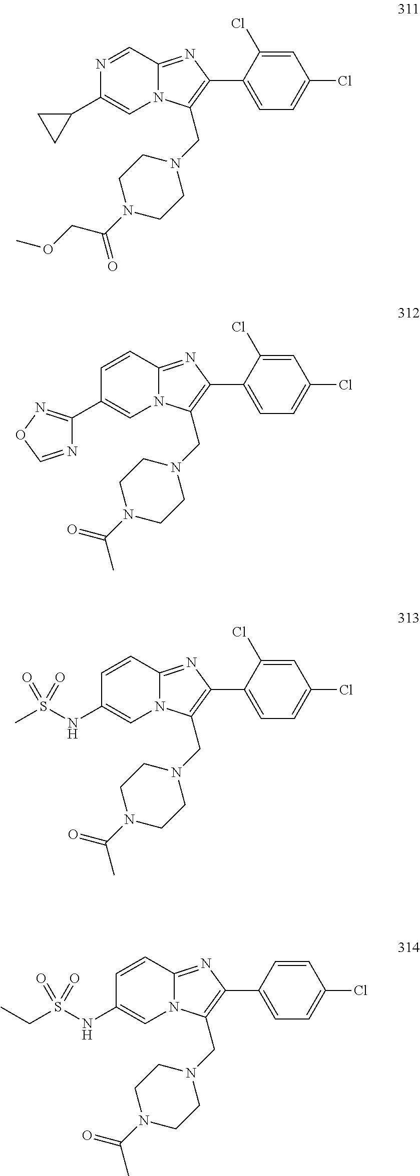 [SMILES: COCC(=O)N1CCN(Cc2c(-c3ccc(Cl)cc3Cl)nc3cnc(C4CC4)cn23)CC1 ; CC(=O)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(NS(C)(=O)=O)cn23)CC1 ; CCS(=O)(=O)Nc1ccc2nc(-c3ccc(Cl)cc3)c(CN3CCN(C(C)=O)CC3)n2c1]

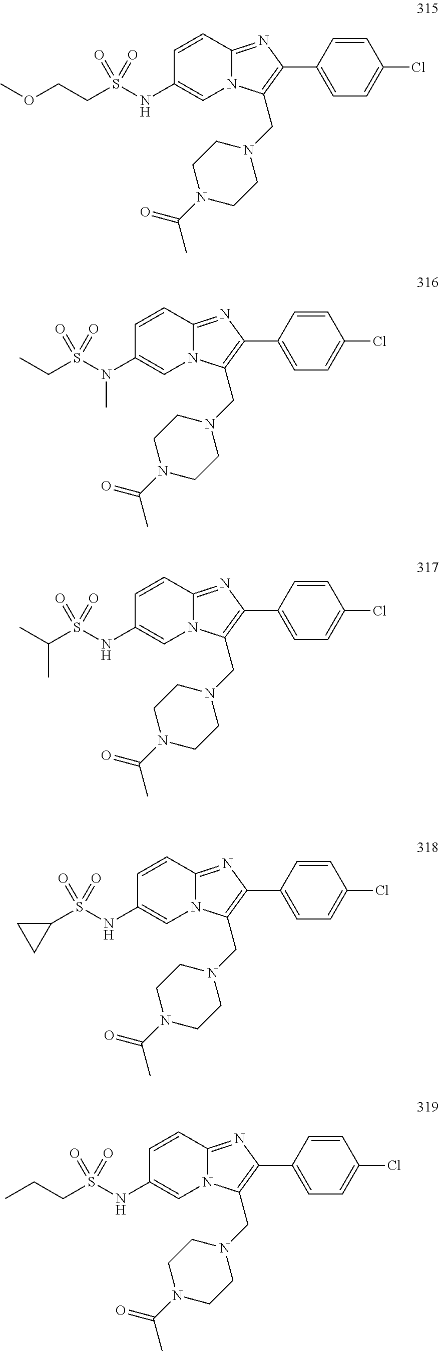 [SMILES: COCCS(=O)(=O)Nc1ccc2nc(-c3ccc(Cl)cc3)c(CN3CCN(C(C)=O)CC3)n2c1 ; CCS(=O)(=O)N(C)c1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(NS(=O)(=O)C(C)C)cn23)CC1 ; CC(=O)N1CCN(Cc2c(-c3ccc(Cl)cc3)nc3ccc(NS(=O)(=O)C4CC4)cn23)CC1 ; CCCS(=O)(=O)Nc1ccc2nc(-c3ccc(Cl)cc3)c(CN3CCN(C(C)=O)CC3)n2c1]

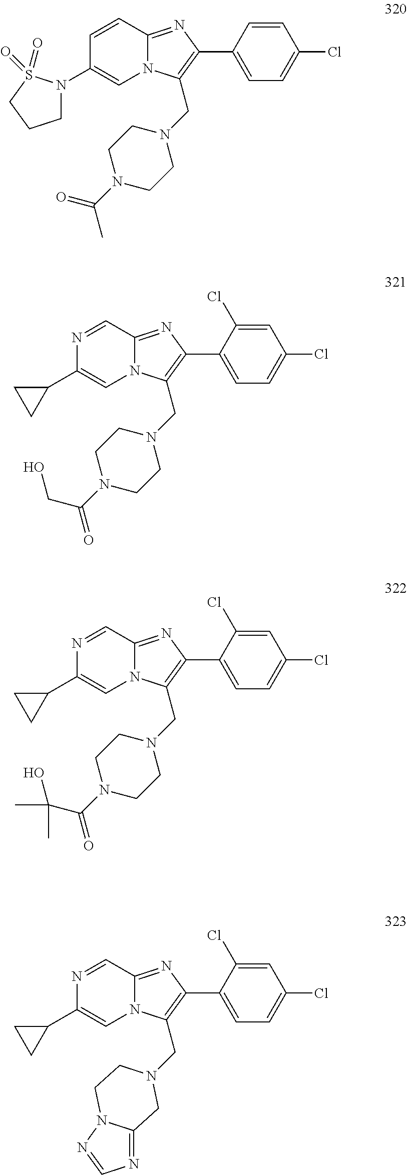 [SMILES: CC(=O)N1CCN(Cc2c(-c3ccc(Cl)cc3)nc3ccc(N4CCCS4(=O)=O)cn23)CC1 ; O=C(CO)N1CCN(Cc2c(-c3ccc(Cl)cc3Cl)nc3cnc(C4CC4)cn23)CC1 ; CC(C)(O)C(=O)N1CCN(Cc2c(-c3ccc(Cl)cc3Cl)nc3cnc(C4CC4)cn23)CC1 ; Clc1ccc(-c2nc3cnc(C4CC4)cn3c2CN2CCn3ncnc3C2)c(Cl)c1]

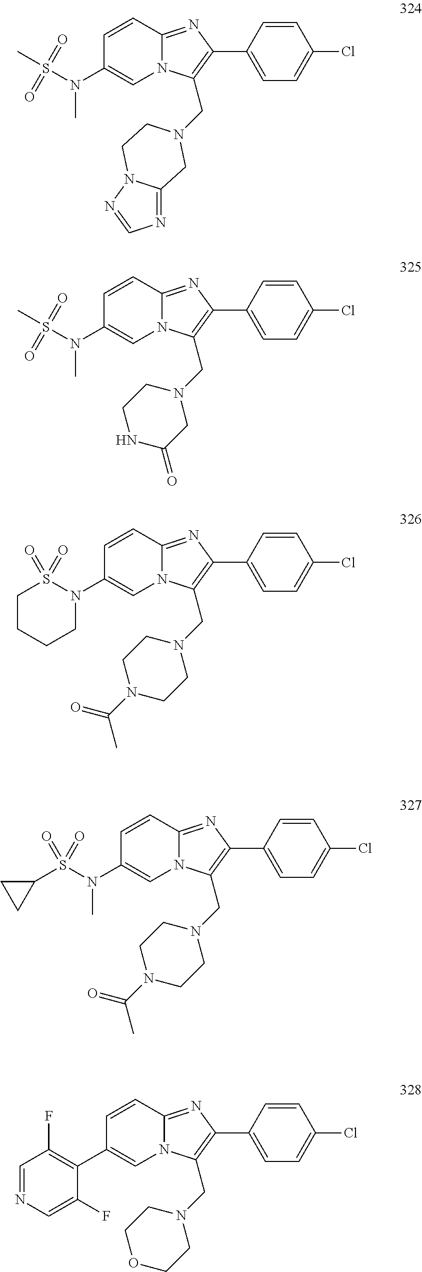

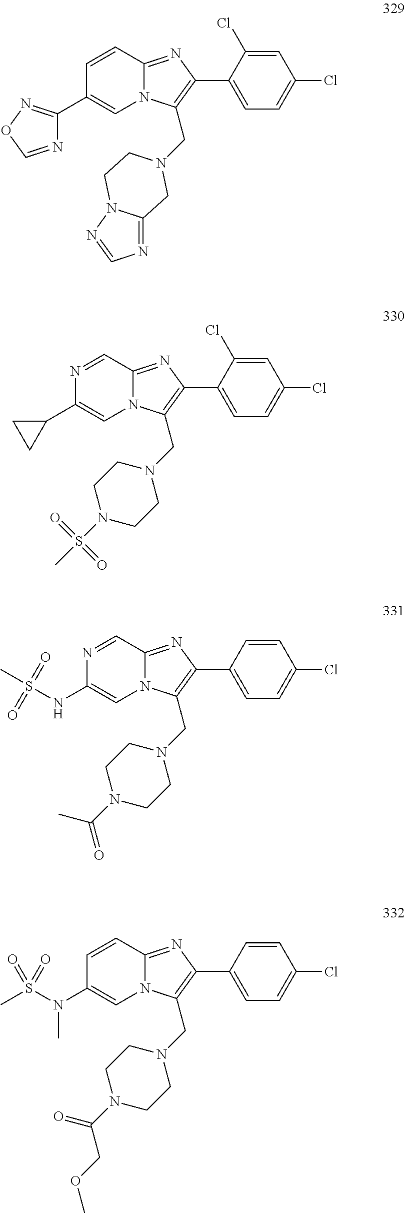 [SMILES: Clc1ccc(-c2nc3ccc(-c4ncon4)cn3c2CN2CCn3ncnc3C2)c(Cl)c1 ; CS(=O)(=O)N1CCN(Cc2c(-c3ccc(Cl)cc3Cl)nc3cnc(C4CC4)cn23)CC1 ; CC(=O)N1CCN(Cc2c(-c3ccc(Cl)cc3)nc3cnc(NS(C)(=O)=O)cn23)CC1 ; COCC(=O)N1CCN(Cc2c(-c3ccc(Cl)cc3)nc3ccc(N(C)S(C)(=O)=O)cn23)CC1]

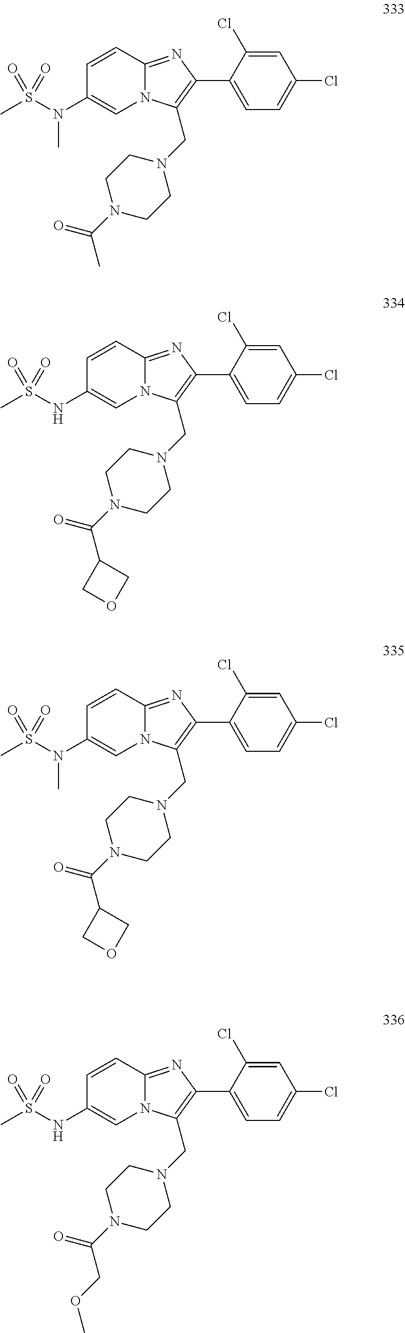 [SMILES: CC(=O)N1CCN(Cc2c(-c3ccc(Cl)cc3Cl)nc3ccc(N(C)S(C)(=O)=O)cn23)CC1 ; CS(=O)(=O)Nc1ccc2nc(-c3ccc(Cl)cc3Cl)c(CN3CCN(C(=O)C4COC4)CC3)n2c1 ; CN(c1ccc2nc(-c3ccc(Cl)cc3Cl)c(CN3CCN(C(=O)C4COC4)CC3)n2c1)S(C)(=O)=O ; COCC(=O)N1CCN(Cc2c(-c3ccc(Cl)cc3Cl)nc3ccc(NS(C)(=O)=O)cn23)CC1]

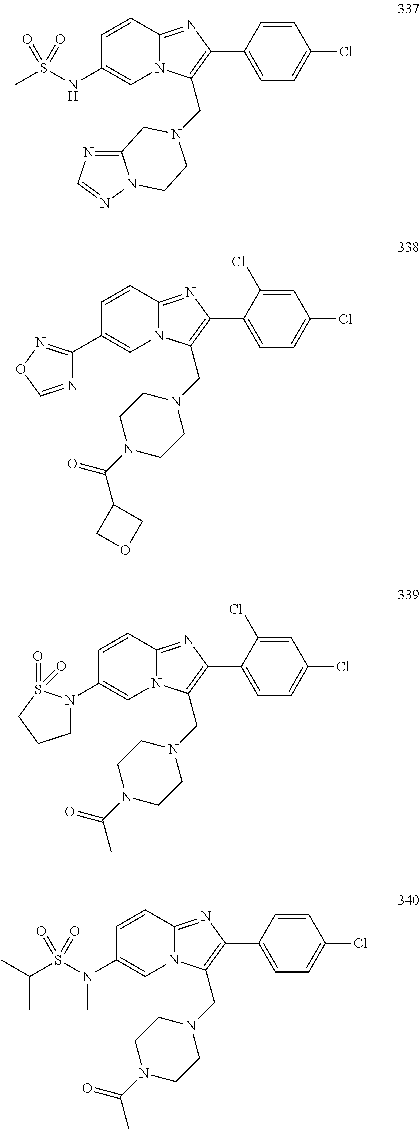 [SMILES: CS(=O)(=O)Nc1ccc2nc(-c3ccc(Cl)cc3)c(CN3CCn4ncnc4C3)n2c1 ; O=C(C1COC1)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(N4CCCS4(=O)=O)cn23)CC1 ; CC(=O)N1CCN(Cc2c(-c3ccc(Cl)cc3)nc3ccc(N(C)S(=O)(=O)C(C)C)cn23)CC1]

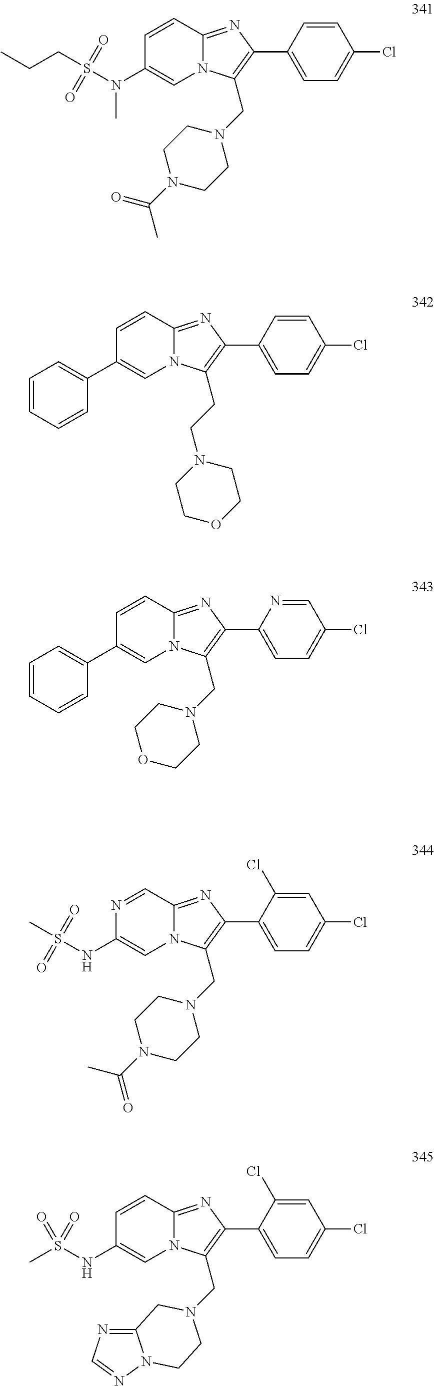 [SMILES: CCCS(=O)(=O)N(C)c1ccc2nc(-c3ccc(Cl)cc3)c(CN3CCN(C(C)=O)CC3)n2c1 ; Clc1ccc(-c2nc3ccc(-c4ccccc4)cn3c2CCN2CCOCC2)cc1 ; Clc1ccc(-c2nc3ccc(-c4ccccc4)cn3c2CN2CCOCC2)nc1 ; CC(=O)N1CCN(Cc2c(-c3ccc(Cl)cc3Cl)nc3cnc(NS(C)(=O)=O)cn23)CC1 ; CS(=O)(=O)Nc1ccc2nc(-c3ccc(Cl)cc3Cl)c(CN3CCn4ncnc4C3)n2c1]

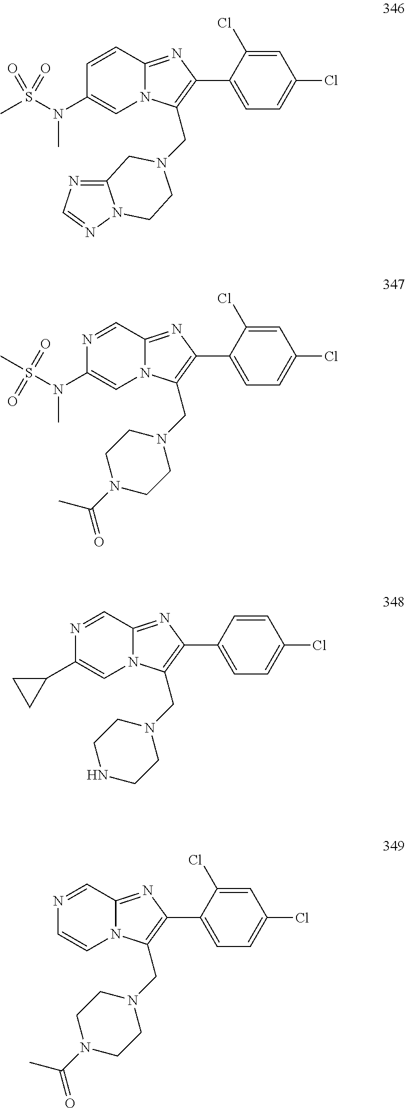 [SMILES: CN(c1ccc2nc(-c3ccc(Cl)cc3Cl)c(CN3CCn4ncnc4C3)n2c1)S(C)(=O)=O ; CC(=O)N1CCN(Cc2c(-c3ccc(Cl)cc3Cl)nc3cnc(N(C)S(C)(=O)=O)cn23)CC1 ; Clc1ccc(-c2nc3cnc(C4CC4)cn3c2CN2CCNCC2)cc1 ; CC(=O)N1CCN(Cc2c(-c3ccc(Cl)cc3Cl)nc3cnccn23)CC1]

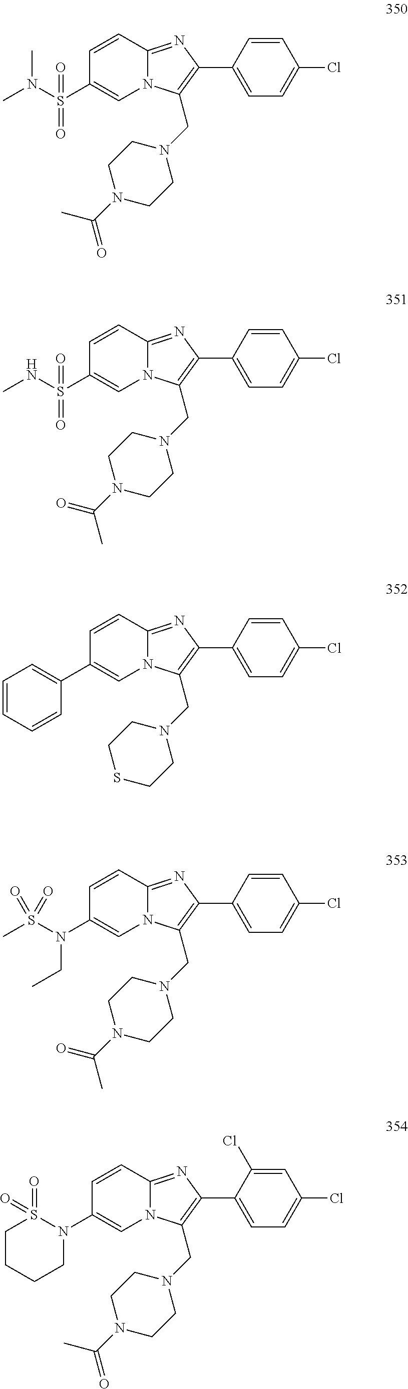 [SMILES: CC(=O)N1CCN(Cc2c(-c3ccc(Cl)cc3)nc3ccc(S(=O)(=O)N(C)C)cn23)CC1 ; CNS(=O)(=O)c1ccc2nc(-c3ccc(Cl)cc3)c(CN3CCN(C(C)=O)CC3)n2c1 ; Clc1ccc(-c2nc3ccc(-c4ccccc4)cn3c2CN2CCSCC2)cc1 ; CCN(c1ccc2nc(-c3ccc(Cl)cc3)c(CN3CCN(C(C)=O)CC3)n2c1)S(C)(=O)=O ; CC(=O)N1CCN(Cc2c(-c3ccc(Cl)cc3Cl)nc3ccc(N4CCCCS4(=O)=O)cn23)CC1]

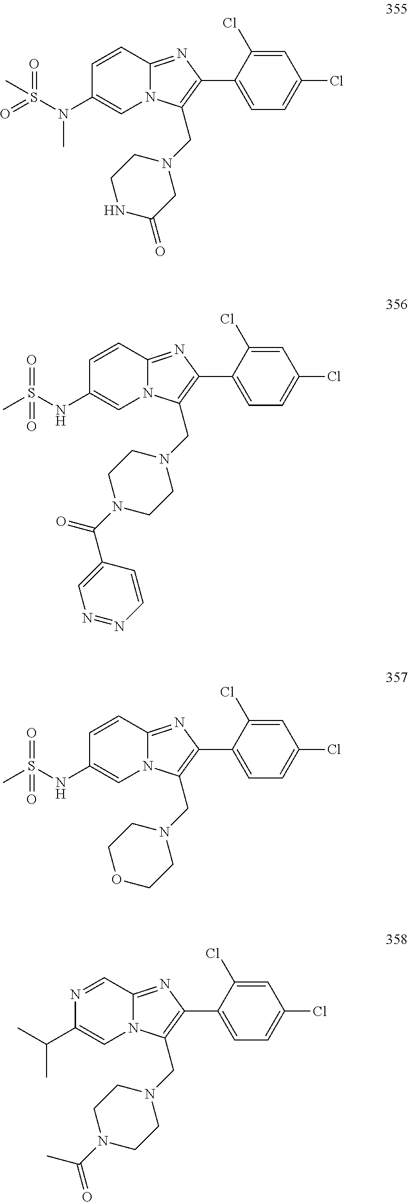 [SMILES: CN(c1ccc2nc(-c3ccc(Cl)cc3Cl)c(CN3CCNC(=O)C3)n2c1)S(C)(=O)=O ; CS(=O)(=O)Nc1ccc2nc(-c3ccc(Cl)cc3Cl)c(CN3CCN(C(=O)c4ccnnc4)CC3)n2c1 ; CS(=O)(=O)Nc1ccc2nc(-c3ccc(Cl)cc3Cl)c(CN3CCOCC3)n2c1 ; CC(=O)N1CCN(Cc2c(-c3ccc(Cl)cc3Cl)nc3cnc(C(C)C)cn23)CC1]

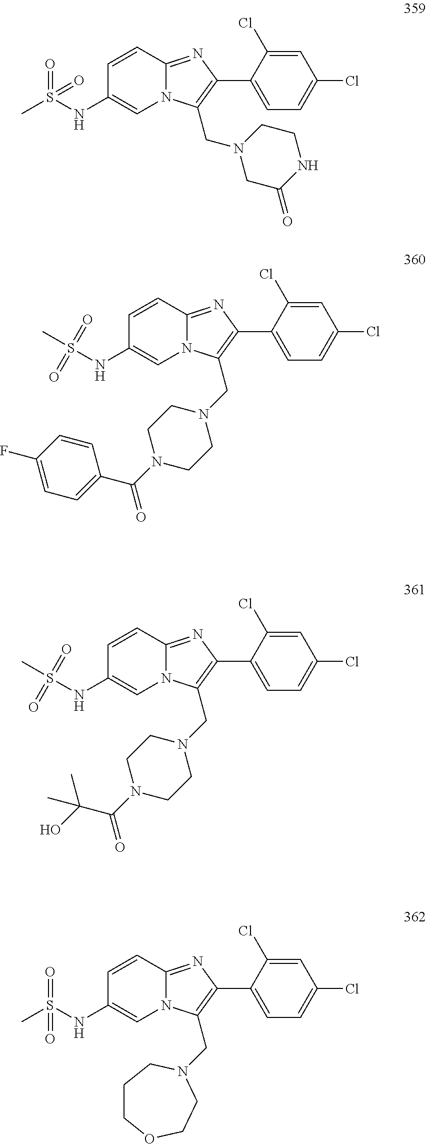 [SMILES: CS(=O)(=O)Nc1ccc2nc(-c3ccc(Cl)cc3Cl)c(CN3CCNC(=O)C3)n2c1 ; CS(=O)(=O)Nc1ccc2nc(-c3ccc(Cl)cc3Cl)c(CN3CCN(C(=O)c4ccc(F)cc4)CC3)n2c1 ; CC(C)(O)C(=O)N1CCN(Cc2c(-c3ccc(Cl)cc3Cl)nc3ccc(NS(C)(=O)=O)cn23)CC1 ; CS(=O)(=O)Nc1ccc2nc(-c3ccc(Cl)cc3Cl)c(CN3CCCOCC3)n2c1]

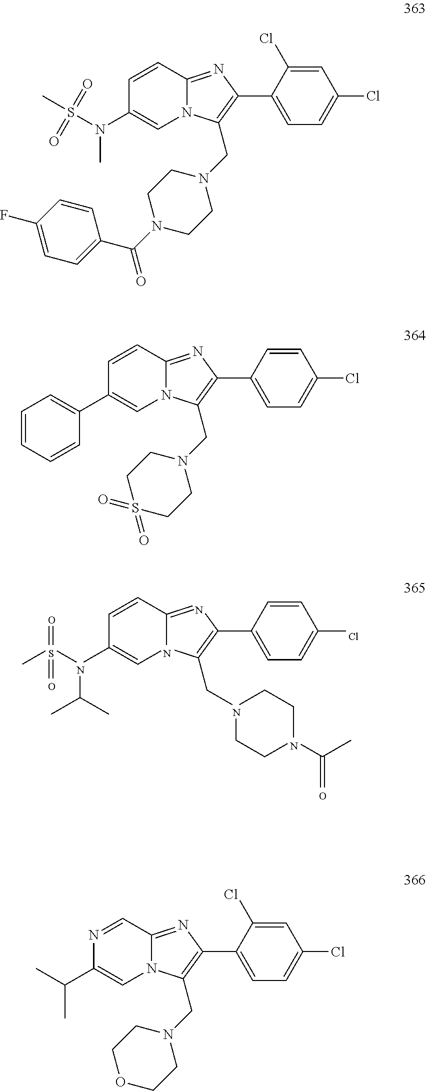

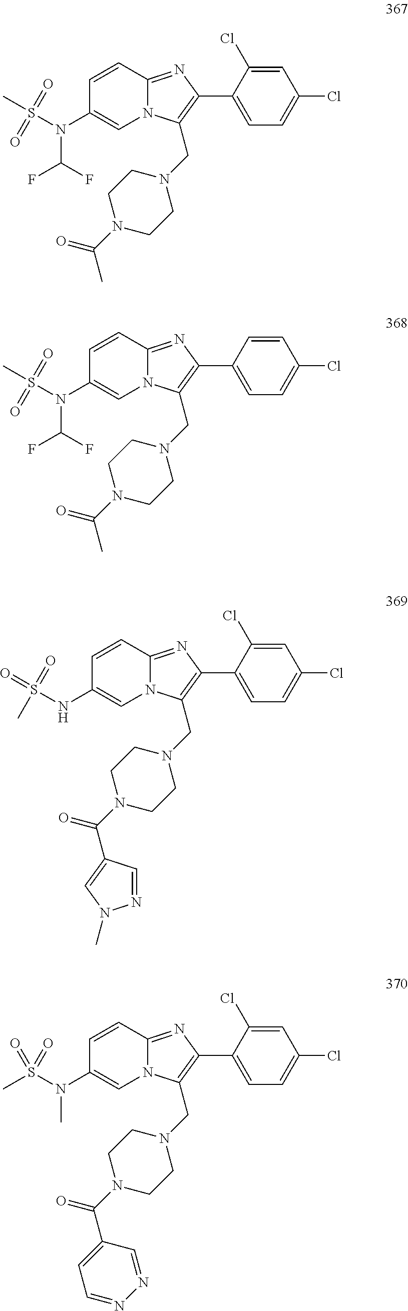 [SMILES: CC(=O)N1CCN(Cc2c(-c3ccc(Cl)cc3Cl)nc3ccc(N(C(F)F)S(C)(=O)=O)cn23)CC1 ; CC(=O)N1CCN(Cc2c(-c3ccc(Cl)cc3)nc3ccc(N(C(F)F)S(C)(=O)=O)cn23)CC1 ; Cn1cc(C(=O)N2CCN(Cc3c(-c4ccc(Cl)cc4Cl)nc4ccc(NS(C)(=O)=O)cn34)CC2)cn1 ; CN(c1ccc2nc(-c3ccc(Cl)cc3Cl)c(CN3CCN(C(=O)c4ccnnc4)CC3)n2c1)S(C)(=O)=O]

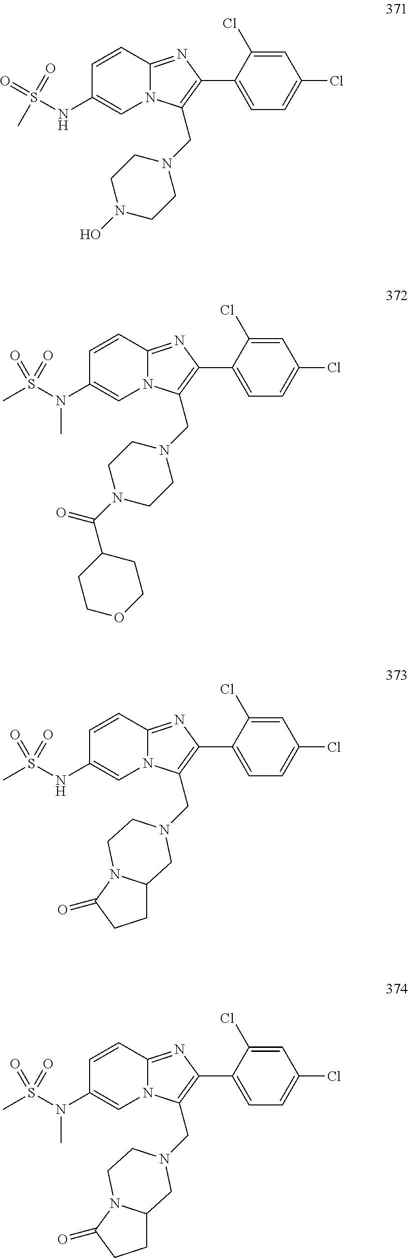 [SMILES: CS(=O)(=O)Nc1ccc2nc(-c3ccc(Cl)cc3Cl)c(CN3CCN(O)CC3)n2c1 ; CN(c1ccc2nc(-c3ccc(Cl)cc3Cl)c(CN3CCN(C(=O)C4CCOCC4)CC3)n2c1)S(C)(=O)=O ; CS(=O)(=O)Nc1ccc2nc(-c3ccc(Cl)cc3Cl)c(CN3CCN4C(=O)CCC4C3)n2c1 ; CN(c1ccc2nc(-c3ccc(Cl)cc3Cl)c(CN3CCN4C(=O)CCC4C3)n2c1)S(C)(=O)=O]

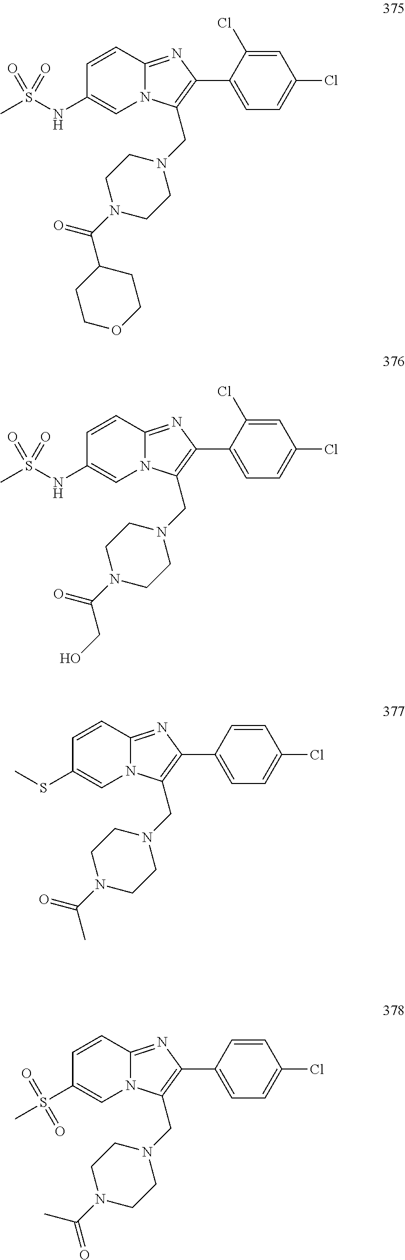 [SMILES: CS(=O)(=O)Nc1ccc2nc(-c3ccc(Cl)cc3Cl)c(CN3CCN(C(=O)C4CCOCC4)CC3)n2c1 ; CS(=O)(=O)Nc1ccc2nc(-c3ccc(Cl)cc3Cl)c(CN3CCN(C(=O)CO)CC3)n2c1 ; CSc1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(S(C)(=O)=O)cn23)CC1]

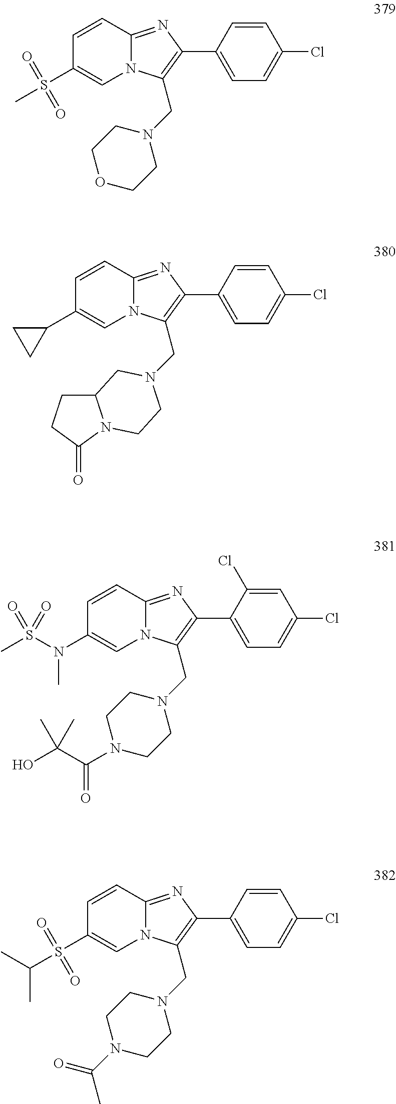 [SMILES: CS(=O)(=O)c1ccc2nc(-c3ccc(Cl)cc3)c(CN3CCOCC3)n2c1 ; O=C1CCC2CN(Cc3c(-c4ccc(Cl)cc4)nc4ccc(C5CC5)cn34)CCN12 ; CN(c1ccc2nc(-c3ccc(Cl)cc3Cl)c(CN3CCN(C(=O)C(C)(C)O)CC3)n2c1)S(C)(=O)=O ; CC(=O)N1CCN(Cc2c(-c3ccc(Cl)cc3)nc3ccc(S(=O)(=O)C(C)C)cn23)CC1]

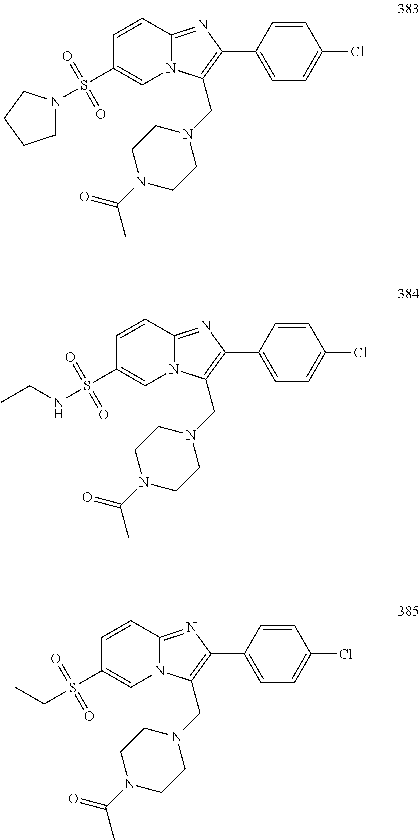 [SMILES: CC(=O)N1CCN(Cc2c(-c3ccc(Cl)cc3)nc3ccc(S(=O)(=O)N4CCCC4)cn23)CC1 ; CCNS(=O)(=O)c1ccc2nc(-c3ccc(Cl)cc3)c(CN3CCN(C(C)=O)CC3)n2c1 ; CCS(=O)(=O)c1ccc2nc(-c3ccc(Cl)cc3)c(CN3CCN(C(C)=O)CC3)n2c1]

37. A compound according to claim 34,
which compound comprises:
an isolated enantiomer, or
a mixture of two or more enantiomers, or
a mixture of two or more diastereomers, and/or epimers, or
a racemic mixture.

38. A method of synthesis of a compound as defined in claim 34, which method comprises reacting a substituted or unsubstituted pyridine compound with a substituted or unsubstituted ketone compound in a ring forming step.

39. A method of synthesis according to claim 38, which method comprises a ring-forming step as follows: 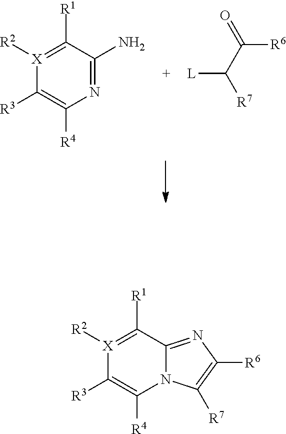
wherein R⁷ comprises the following group: 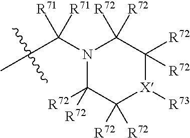
wherein L is a leaving group; X is C or N; X′ is C, N, or S; and wherein R¹, R², R³, R⁴, and R⁶ are independently selected from H or an organic group; and wherein when X is NR² is absent; optionally wherein when any of R¹, R², R³, R⁴, R⁶ and R⁷ is H, the method comprises a further step of substituting that H with an organic group; preferably
wherein R³ is H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl); preferably a C₃-C₆ alkyl group (such as propyl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃ and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5, or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe,
and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-C₁-Ph-, 3-Cl-Ph-, 4-C₁-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pre-Ph-, 3,(4 or 5)-Bu₂-Ph-, 3,(4 or 5)-(CN)₂-Ph-, 3,(4 or 5 NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R³ is selected from the following:
an unsubstituted, 2-monosubstituted, or 2,6-disubstituted phenyl group, preferably where the substituent is selected from F, Cl and CN (such as Ph-, 2-F-Ph-, 2,6-F₂-Ph-, 2-Cl-Ph-, 2,6-Cl₂-Ph-, 2-CN-Ph-, 2,6-(CN)₂-Ph-, 2,6-F, CN-Ph-, 2,6-F, Cl-Ph-, and 2,6-Cl, CN-Ph-);
an unsubstituted or 3-monosubstituted pyridine-2-yl, or a 2,6-disubstituted Pyridine-4-yl group, preferably where the substituent is selected from F, Cl and CN (such as Pyr-2-yl, Pyr-3-yl, Pyr-4-yl, 3-F-Pyr-2-yl, 3-Cl-Pyr-2-yl-, 3-CN-Pyr-2-yl, 2,6-F₂-Pyr-4-yl, 2,6-Cl₂-Pyr-4-yl and 2,6-(CN)₂-Pyr-4-yl;
an unsubstituted or 4-substituted pyrid-1,2-azine-3-yl group, preferably where the substituent is selected from F, Cl and CN (such as pyridazine-3-yl, 4-F-pyridazine-3-yl, 4-Cl-pyridazine-3-yl, and 4-CN-pyridazine-3-yl);
a substituted or unsubstituted 1,2,4-oxadiazol-3-yl group;
a cyclic aminosulphonyl- group (such as —N(SO₂)(CH₂)₃ and —N(SO₂)(CH₂)₄);
a linear or branched aminosulphonyl group (such as —NH—SO₂-Me, —NH—SO₂-Et, —NH—SO₂-iPr, —NH—SO₂-cycloPr, —NH—SO₂—Pr, —NH—SO₂-EtOMe, —NMe-SO₂-Me, —NMe-SO₂-Et, —NMe-SO₂-iPr, —NMe-SO₂-cycloPr, —NMe-SO₂—Pr, —NMe-SO₂-EtOMe, —NEt-SO₂-Me, —NEt-SO₂-Et, —NEt-SO₂-iPr, —NEt-SO₂-cycloPr, —NEt-SO₂—Pr, —NEt-SO₂-EtOMe, —NiPr—SO₂-Me, —NiPr—SO₂-Et, —NiPr—SO₂-iPr, —NiPr—SO₂-cycloPr, —NiPr—SO₂—Pr, —NiPr—SO₂-EtOMe, —N(CHF₂)—SO₂-Me, —N(CHF₂)—SO₂-Et, —N(CHF₂)—SO₂-iPr, —N(CHF₂)—SO₂-cycloPr, —N(CHF₂)—SO₂—Pr, and —N(CHF₂)—SO₂-EtOMe;
a linear or branched sulphonylamino group (such as —SO₂—NH₂, —SO₂—NHMe, —SO₂—NHEt, —SO₂—NMe₂, —SO₂—NMeEt, —SO₂—NHEt₂, and —SO₂-pyrrolidin-N-yl);
a linear or branched sulphonyl group (such as —SO₂Me, —SO₂Et, —SO₂Pr, —SO₂iPr);
a thioether group (such as —SMe, —SEt, —SPr, and SiPr); and
an isopropyl, cyclopropyl and a propen-2-yl group;
wherein R⁶ is H or a group selected from the following groups:
a halogen (such as F, Cl, Br and I);
a linear or branched C₁-C₆ alkyl group (such as methyl (Me), ethyl (Et), propyl (Pr), iso-propyl (i-Pr), n-butyl (n-Bu), iso-butyl (i-Bu), tert-butyl (t-Bu), pentyl and hexyl),
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂—NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)OPr-Ph, —NH-(2,3 or 4)OBu-Ph, —NH-2,(3,4,5, or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH)₂-Ph-, 2,(3,4,5 or 6)-(MeO)₂-Ph-, 2,(3, 4, 5, or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-), and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl);
preferably wherein R⁶ comprises—an aromatic group selected from Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3,4,5 or 6)-(NO₂)₂-Ph-, 2,(3,4,5 or 6)-(NH₂)₂-Ph-, 2,(3,4,5 or 6)-(MeO)₂-Ph-, 2,(3,4,5 or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-3,(4 or 5)-Et₂-Ph-, 3,(4 or 5)-Pre-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂)-Ph-, 3-(NH₂)-Ph-, 4-(NH₂)-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-,
particularly preferably wherein R⁶ is selected from the following:
an unsubstituted, 2-, 3- or 4-monosubstituted, and a 2,4 or 3,4-disubstituted phenyl group, preferably wherein the substituent is F, Cl or —OMe (such as 2-F-Ph, 2-Cl-Ph, 2-OMe-Ph, 3-F-Ph, 3-Cl-Ph, 3-OMe-Ph, 4-F-Ph, 4-Cl-Ph, 4-OMe-Ph, 2,4-F₂-Ph, 2,4-Cl₂-Ph, 2,4-(OMe)₂-Ph, 3,4-F₂-Ph, 3,4-Cl₂-Ph, and 3,4-(OMe)₂-Ph);
a substituted or unsubstituted pyridine-2-yl, pyridine-3-yl or pyridine-4-yl group preferably wherein the substituent is F or CI (such as pyridine-2-yl, pyridine-3-yl, pyridine-4-yl, 3-F-pyridine-2-yl, 3-Cl-pyridine-2-yl, 4-F-pyridine-2-yl, 4-Cl-pyridine-2-yl, 5-F-pyridine-2-yl, 5-Cl-pyridine-2-yl; and
a cyclic ether group (such as tetrahydrofuran-2-yl, tetrahydrofuran-3-yl, terahydropyran-2-yl, tetrahydropyran-3-yl and tetrahydropyran-4-yl),
wherein R¹, R², and R⁴ are each independently H or a group selected from the following groups:
a halogen (such as F, Cl, Br and I);
a linear or branched C₁-C₆ alkyl group (such as methyl (Me), ethyl (Et), propvl (Pr), iso-propyl (i-Pr), n-butyl (n-Bu), iso-butyl (i-Bu), tert-butyl (t-Bu), pentyl and hexyl);
a linear or branched C₁-C₆ alkyl-aryl group (such as —CH₂Ph, —CH₂(2,3 or 4)F-Ph, —CH₂(2,3 or 4)Cl-Ph, —CH₂(2,3 or 4)Br-Ph, —CH₂(2,3 or 4)I-Ph, —CH₂CH₂Ph, —CH₂CH₂CH₂Ph, —CH₂CH₂CH₂CH₂Ph, —CH₂CH₂CH₂CH₂CH₂Ph, and —CH₂CH₂CH₂CH₂CH₂CH₂Ph);
a linear or branched C₁-C₆ halogenated alkyl group (such as —CH₂F, —CH₂Cl, —CH₂Br, —CH₂I, —CF₃, —CCl₃, —CBr₃, —Cl₃, —CH₂CF₃, —CH₂CCl₃, —CH₂CBr₃, and —CH₂Cl₃);
a linear or branched primary secondary or tertiary C₁-C₆ amine group (such as —NH₂, —NMeH, —NMe₂, —NEtH, —NEtMe, —NEt₂, —NPrH, —NPrMe, —NPrEt, —NPr₂, —NBuH, —NBuMe, —NBuEt, —CH₂—NH₂, —CH₂—NMeH, —CH₂—NMe₂, —CH₂—NEtH, —CH₂-NEtMe, —CH₂—NEt₂, —CH₂—NPrH, —CH₂—NPrMe, and —CH₂—NPrEt);
a amino-aryl group (such as —NH-Ph, —NH-(2,3 or 4)F-Ph, —NH-(2,3 or 4)Cl-Ph, —NH-(2,3 or 4)Br-Ph, —NH-(2,3 or 4)I-Ph, —NH-(2,3 or 4)Me-Ph, —NH-(2,3 or 4)Et-Ph, —NH-(2,3 or 4)Pr-Ph, —NH-(2,3 or 4)Bu-Ph, NH-(2,3 or 4)OMe-Ph, —NH-(2,3 or 4)OEt-Ph, —NH-(2,3 or 4)Pr-Ph, —NH-(2,3 or 4)OBu-Ph, —NH-2,(3, 4, 5, or 6)F₂-Ph, —NH-2,(3,4,5 or 6)Cl₂-Ph, —NH-2,(3,4,5 or 6)Br₂-Ph, —NH-2,(3,4,5 or 6)I₂-Ph, —NH-2,(3,4,5 or 6)Me₂-Ph, —NH-2,(3,4,5 or 6)Et₂-Ph, —NH-2,(3,4,5, or 6)Pr₂-Ph, —NH-2,(3,4,5 or 6)Bu₂-Ph;
a cyclic amine or amido group (such as pyrrolidin-1-yl, pyrrolidin-2-yl, pyrrolidin-3-yl, piperidin-1-yl, piperidin-2-yl, piperidin-3-yl, piperidin-4-yl, morpholin-1-yl, morpholin-2-yl, morpholin-3-yl, morpholin-4-yl, 2-keto-pyrrolidinyl, 3-keto-pyrrolidinyl, 2-keto-piperidinyl, 3-keto-piperidinyl, and 4-keto-piperidinyl);
a cyclic C₃-C₈ alkyl group (such as cyclopropyl, cyclobutyl, cyclopentyl, cyclohexyl, cycloheptyl and cyclooctyl);
a linear or branched C₁-C₆ alcohol group (such as —CH₂OH, —CH₂CH₂OH, —CH₂CH₂CH₂OH, —CH₂CH₂CH₂CH₂OH, —CH₂CH₂CH₂CH₂CH₂OH, and —CH₂CH₂CH₂CH₂CH₂CH₂OH);
a linear or branched C₁-C₆ carboxylic acid group (such as —COOH, —CH₂COOH, —CH₂CH₂COOH, —CH₂CH₂CH₂COOH, —CH₂CH₂CH₂CH₂COOH, and —CH₂CH₂CH₂CH₂CH₂COOH);
a linear or branched carbonyl group (such as —(CO)Me, —(CO)Et, —(CO)Pr, —(CO)iPr, —(CO)nBu, —(CO)iBu, —(CO)tBu, —(CO)Ph, —(CO)CH₂Ph, —(CO)CH₂OH, —(CO)CH₂OCH₃, —(CO)CH₂NH₂, —(CO)CH₂NHMe, —(CO)CH₂NMe₂, —(CO)-cyclopropyl, —(CO)-1,3-epoxypropan-2-yl; —(CO)NH₂, —(CO)NHMe, —(CO)NMe₂, —(CO)NHEt, —(CO)NEt₂, —(CO)-pyrollidine-N-yl, —(CO)-morpholine-N-yl, —(CO)-piperazine-N-yl, —(CO)—N-methyl-piperazine-N-yl, —(CO)NHCH₂CH₂OH, —(CO)NHCH₂CH₂OMe, —(CO)NHCH₂CH₂NH₂, —(CO)NHCH₂CH₂NHMe, and —(CO)NHCH₂CH₂NMe₂;
a linear or branched C₁-C₆ carboxylic acid ester group (such as —COOMe, —COOEt, —COOPr, —COO-i-Pr, —COO-n-Bu, —COO-i-Bu, —COO-t-Bu, —CH₂COOMe, —CH₂CH₂COOMe, —CH₂CH₂CH₂COOMe, and —CH₂CH₂CH₂CH₂COOMe);
a linear or branched C₁-C₆ amide group (such as —CO—NH₂, —CO—NMeH, —CO—NMe₂, —CO—NEtH, —CO—NEtMe, —CO—NEt₂, —CO—NPrH, —CO—NPrMe, and —CO—NPrEt);
a linear or branched C₁-C₇ amino carbonyl group (such as —NH—CO-Me, —NH—CO-Et, —NH—CO—Pr, —NH—CO—Bu, —NH—CO-pentyl, —NH—CO-hexyl, —NH—CO-Ph, —NMe-CO-Me, —NMe-CO-Et, —NMe-CO—Pr, —NMe-CO—Bu, —NMe-CO-pentyl, —NMe-CO-hexyl, —NMe-CO-Ph;
a linear or branched C₁-C₇ alkoxy or aryloxy group (such as —OMe, —OEt, —OPr, —O-i-Pr, —O-n-Bu, —O-i-Bu, —O-t-Bu, —O-pentyl, —O-hexyl, —OCH₂F, —OCHF₂, —OCF₃, —OCH₂Cl, —OCHCl₂, —OCCl₃, —O-Ph, —O—CH₂-Ph, —O—CH₂-(2,3 or 4)-F-Ph, —O—CH₂-(2,3 or 4)-Cl-Ph, —CH₂OMe, —CH₂OEt, —CH₂OPr, —CH₂OBu, —CH₂CH₂OMe, —CH₂CH₂CH₂OMe, —CH₂CH₂CH₂CH₂OMe, and —CH₂CH₂CH₂CH₂CH₂OMe);
a linear or branched aminoalkoxy group (such as —OCH₂NH₂, —OCH₂NHMe, —OCH₂NMe₂, —OCH₂NHEt, —OCH₂NEt₂, —OCH₂CH₂NH₂, —OCH₂CH₂NHMe, —OCH₂CH₂NMe₂, —OCH₂CH₂NHEt, and —OCH₂CH₂NEt₂;
a sulphonyl group (such as —SO₂Me, —SO₂Et, —SO₂Pr, —SO₂iPr, —SO₂Ph, —SO₂-(2,3 or 4)-F-Ph, —SO₂-cyclopropyl, —SO₂CH₂CH₂OCH₃;
a sulphonylamino group (such as —SO₂NH₂, —SO₂NHMe, —SO₂NMe₂, —SO₂NHEt, —SO₂NEt₂, —SO₂-pyrrolidine-N-yl, —SO₂-morpholine-N-yl, —SO₂NHCH₂OMe, and —SO₂NHCH₂CH₂OMe);
an aminosulphonyl group (such as —NHSO₂Me, —NHSO₂Et, —NHSO₂Pr, —NHSO₂iPr, —NHSO₂Ph, —NHSO₂-(2,3 or 4)-F-Ph, —NHSO₂-cyclopropyl, —NHSO₂CH₂CH₂OCH₃);
a cyclic aminosulphonyl- group (such as —N(SO₂)(CH₂)₃ and —N(SO₂)(CH₂)₄);
an aromatic group (such as Ph-, 2-F-Ph-, 3-F-Ph-, 4-F-Ph-, 2-Cl-Ph-, 3-Cl-Ph-, 4-Cl-Ph-, 2-Br-Ph-, 3-Br-Ph-, 4-Br-Ph-, 2-I-Ph-, 3-I-Ph, 4-I-Ph-, 2,(3,4,5 or 6)-F₂-Ph-, 2,(3,4,5 or 6)-Cl₂-Ph-, 2,(3,4,5 or 6)-Br₂-Ph-, 2,(3,4,5 or 6)-I₂-Ph-, 2,(3,4,5 or 6)-Me₂-Ph-, 2,(3,4,5 or 6)-Et₂-Ph-, 2,(3,4,5 or 6)-Pr₂-Ph-, 2,(3,4,5 or 6)-Bu₂-Ph-, 2,(3,4,5 or 6)-(CN)₂-Ph-, 2,(3, 4, 5, or 6)-(NO₂)₂-Ph-, 2,(3, 4, 5, or 6)-(NH₂)₂-Ph-, 2,(3, 4, 5, or 6)-(MeO)₂-Ph-, 2,(3, 4, 5, or 6)-(CF₃)₂-Ph-, 3,(4 or 5)-F₂-Ph-, 3,(4 or 5)-Cl₂-Ph-, 3,(4 or 5)-Br₂-Ph-, 3,(4 or 5)-I₂-Ph-, 3,(4 or 5)-Me₂-Ph-, 3,(4 or 5)-Et₂-Ph-, 3,(4 or 5)-Pr₂-Ph-, 3,(4 or 5)-Bu₂-Ph-, 3,(4 or 5)-(CN)₂-Ph-, 3,(4 or 5)-(NO₂)₂-Ph-, 3,(4 or 5)-(NH₂)₂-Ph-, 3,(4 or 5)-(MeO)₂-Ph-, 3,(4 or 5)-(CF₃)₂-Ph-, 2-Me-Ph-, 3-Me-Ph-, 4-Me-Ph-, 2-Et-Ph-, 3-Et-Ph-, 4-Et-Ph-, 2-Pr-Ph-, 3-Pr-Ph-, 4-Pr-Ph-, 2-Bu-Ph-, 3-Bu-Ph-, 4-Bu-Ph-, 2-(CN)-Ph-, 3-(CN)-Ph-, 4-(CN)-Ph-, 2-(NO₂)-Ph-, 3-(NO₂)-Ph-, 4-(NO₂)-Ph-, 2-(NH₂) Ph-, 3-(NH₂)-Ph-, 4-(NH₂-Ph-, 2-MeO-Ph-, 3-MeO-Ph-, 4-MeO-Ph-, 2-(NH₂—CO)-Ph-, 3-(NH₂—CO)-Ph-, 4-(NH₂—CO)-Ph-, 2-CF₃-Ph-, 3-CF₃-Ph-, 4-CF₃-Ph-, 2-CF₃O-Ph-, 3-CF₃O-Ph-, and 4-CF₃O-Ph-); and
a saturated or unsaturated heterocyclic group including an aromatic heterocyclic group (such as pyridin-1-yl, pyridin-2-yl pyridin-3-yl pyridin-4-yl, thiphen-1-yl, thiphen-2-yl, thiphen-3-yl, pyrimidin-1-yl, pyrimidin-2-yl, pyrimidin-3-yl, pyrimidin-4-yl, pyrimidin-5-yl, pyrimidin-6-yl, tetrazole-1 yl, tetrazole-2-yl, tetrazole-3-yl, tetrazole-4-yl, tetrazole-5-yl, (1,3,4-oxadiazol)-1-yl, (1,3,4-oxadiazol)-2-yl, (1,3,4-oxadiazol)-3-yl, (1,3,4-oxadiazol)-4-yl, (1,3,4-oxadiazol)-5-yl, (1,2,4-oxadiazol)-1-yl, (1,2,4-oxadiazol)-2-yl, (1,2,4-oxadiazol)-3-yl, (1,2,4-oxadiazol)-4-yl, (1,2,4-oxadiazol)-5-yl, (1,3-thiazol)-1-yl, (1,3-thiazol)-2-yl, (1,3-thiazol)-3-yl, (1,3-thiazol)-4-yl, (1,3-thiazol)-5-yl, furan-1-yl, furan-2-yl, and furan-3-yl).

40. The method according to claim 38, wherein L is selected from the following:
a halogen (such as F, Cl, Br, and I);
an OH and an alkoxy group (such as —OMe, —OEt, —OPr, and —OPh);
N₂⁺, OR′₂⁺, —OSO₂C₄F₉, —OSO₂CF₃, —OSO₂F, a tosylate (—OTs), a mesylate (—OMs), —OH₂⁺, an acyl halide (such as —CO—F, —CO—Cl, —CO—Br, —CO—I), —OH(C₁-C₆ alkyl)⁺, —ONO₂, —OPO(OH)₂, an inorganic ester, —S(C₁-C₆ alkyl)₂⁺, —N(C₁-C₆ alkyl)₃⁺, —OCO(C₁-C₆ alkyl), and —NH₃⁺.

41. The method according to claim 38, wherein the ring-forming step is carried out by refluxing under acid or base catalysis.

42. The method according to claim 38, which method comprises the following steps: 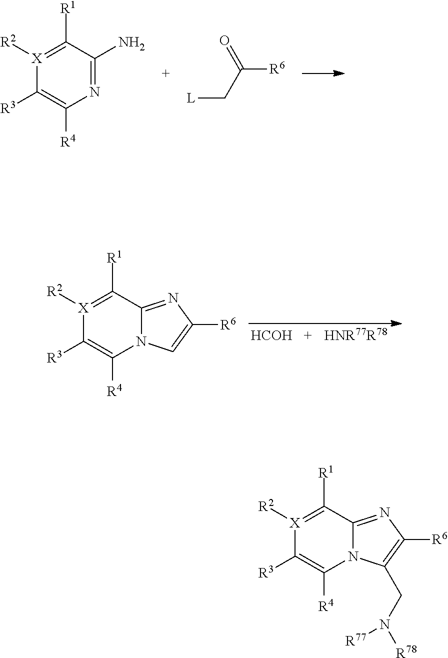
wherein R⁷⁷ and R⁷⁸ may alone or together form any of the compounds listed below:

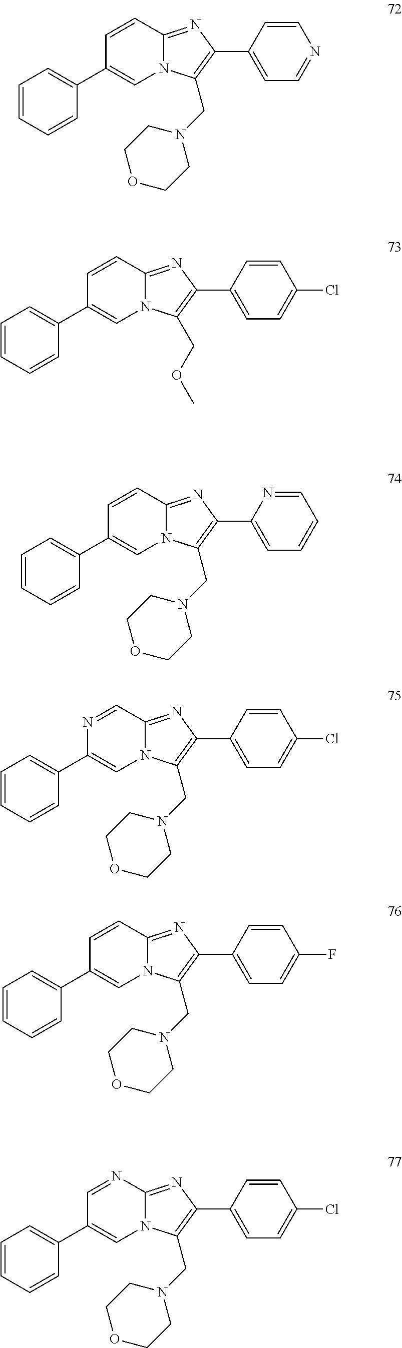 [SMILES: c1ccc(-c2ccc3nc(-c4ccncc4)c(CN4CCOCC4)n3c2)cc1 ; COCc1c(-c2ccc(Cl)cc2)nc2ccc(-c3ccccc3)cn12 ; c1ccc(-c2ccc3nc(-c4ccccn4)c(CN4CCOCC4)n3c2)cc1 ; Clc1ccc(-c2nc3cnc(-c4ccccc4)cn3c2CN2CCOCC2)cc1 ; Fc1ccc(-c2nc3ccc(-c4ccccc4)cn3c2CN2CCOCC2)cc1 ; Clc1ccc(-c2nc3ncc(-c4ccccc4)cn3c2CN2CCOCC2)cc1]

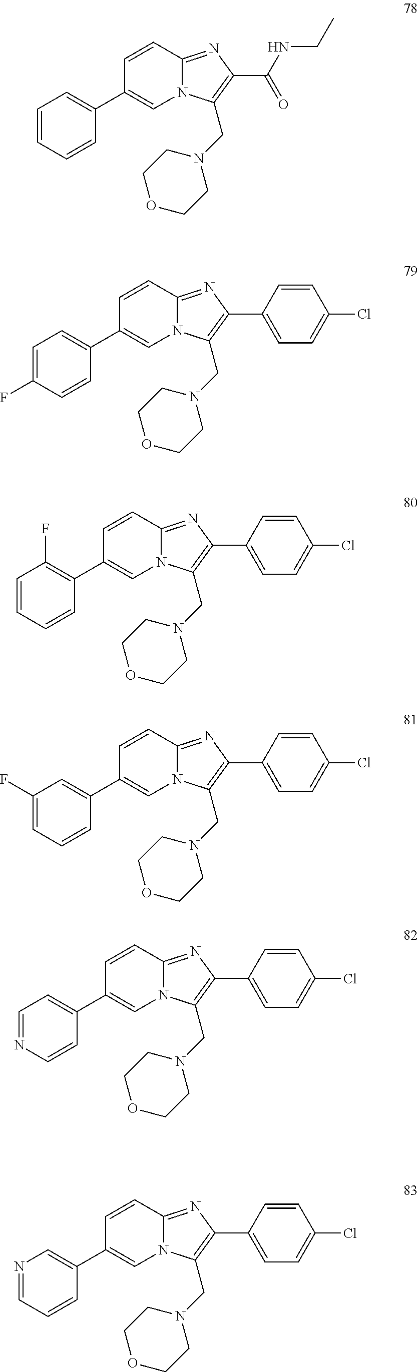 [SMILES: CCNC(=O)c1nc2ccc(-c3ccccc3)cn2c1CN1CCOCC1 ; Fc1ccc(-c2ccc3nc(-c4ccc(Cl)cc4)c(CN4CCOCC4)n3c2)cc1 ; Fc1ccccc1-c1ccc2nc(-c3ccc(Cl)cc3)c(CN3CCOCC3)n2c1 ; Fc1cccc(-c2ccc3nc(-c4ccc(Cl)cc4)c(CN4CCOCC4)n3c2)c1 ; Clc1ccc(-c2nc3ccc(-c4ccncc4)cn3c2CN2CCOCC2)cc1 ; Clc1ccc(-c2nc3ccc(-c4cccnc4)cn3c2CN2CCOCC2)cc1]

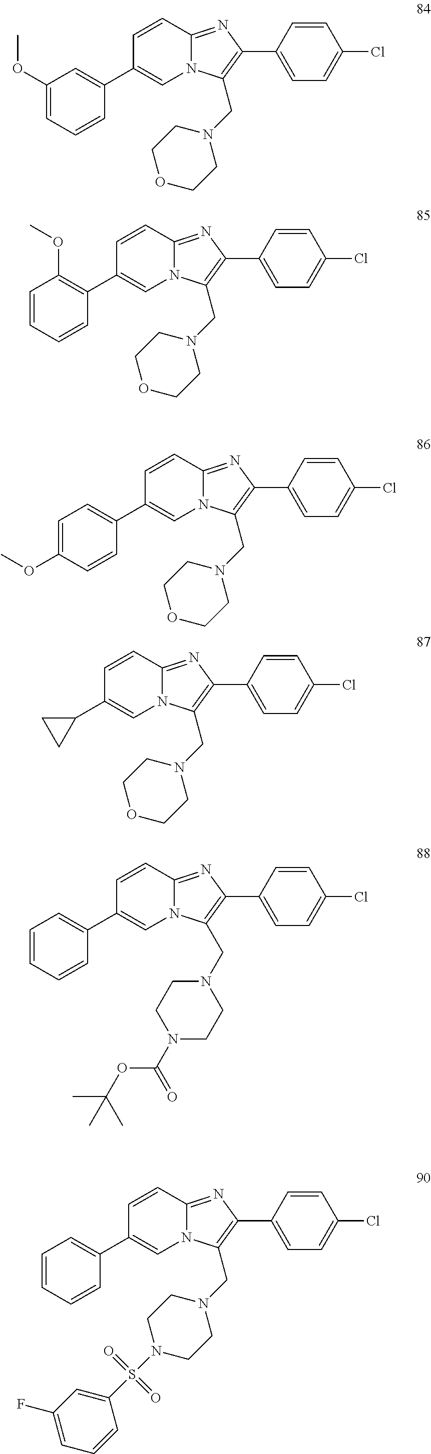

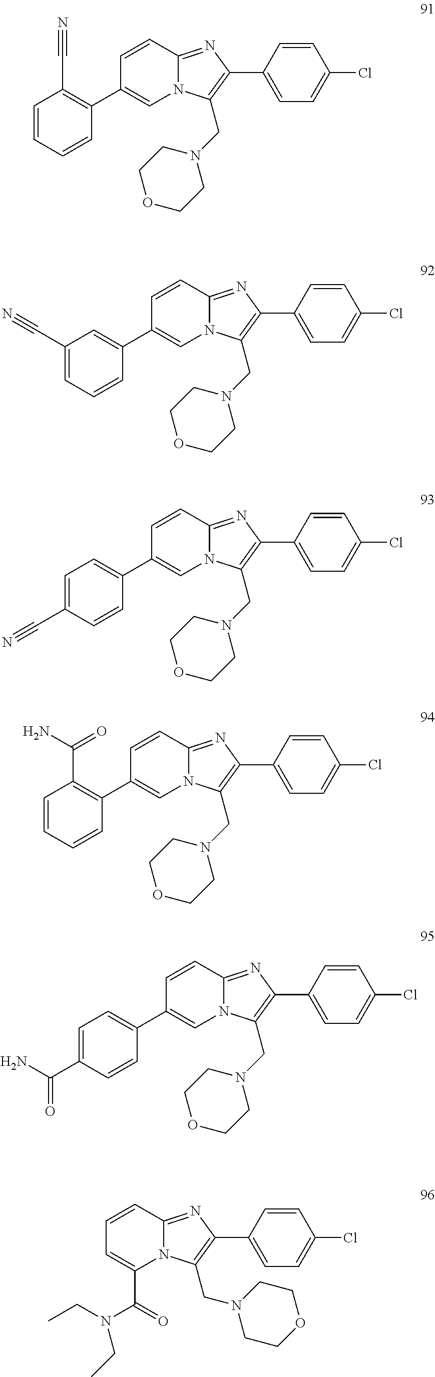 [SMILES: N#Cc1ccccc1-c1ccc2nc(-c3ccc(Cl)cc3)c(CN3CCOCC3)n2c1 ; N#Cc1cccc(-c2ccc3nc(-c4ccc(Cl)cc4)c(CN4CCOCC4)n3c2)c1 ; N#Cc1ccc(-c2ccc3nc(-c4ccc(Cl)cc4)c(CN4CCOCC4)n3c2)cc1 ; NC(=O)c1ccccc1-c1ccc2nc(-c3ccc(Cl)cc3)c(CN3CCOCC3)n2c1 ; NC(=O)c1ccc(-c2ccc3nc(-c4ccc(Cl)cc4)c(CN4CCOCC4)n3c2)cc1 ; CCN(CC)C(=O)c1cccc2nc(-c3ccc(Cl)cc3)c(CN3CCOCC3)n12]

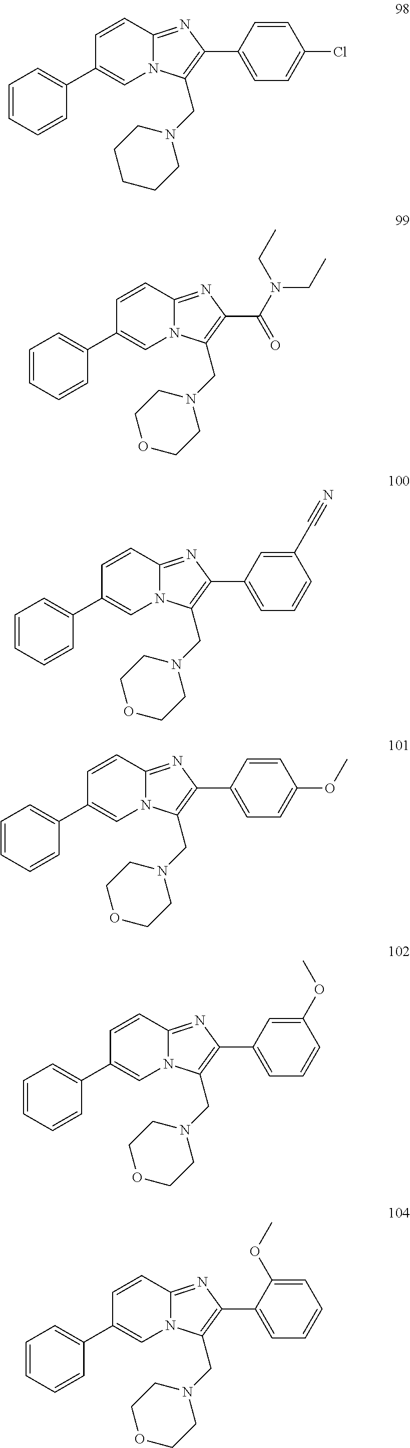

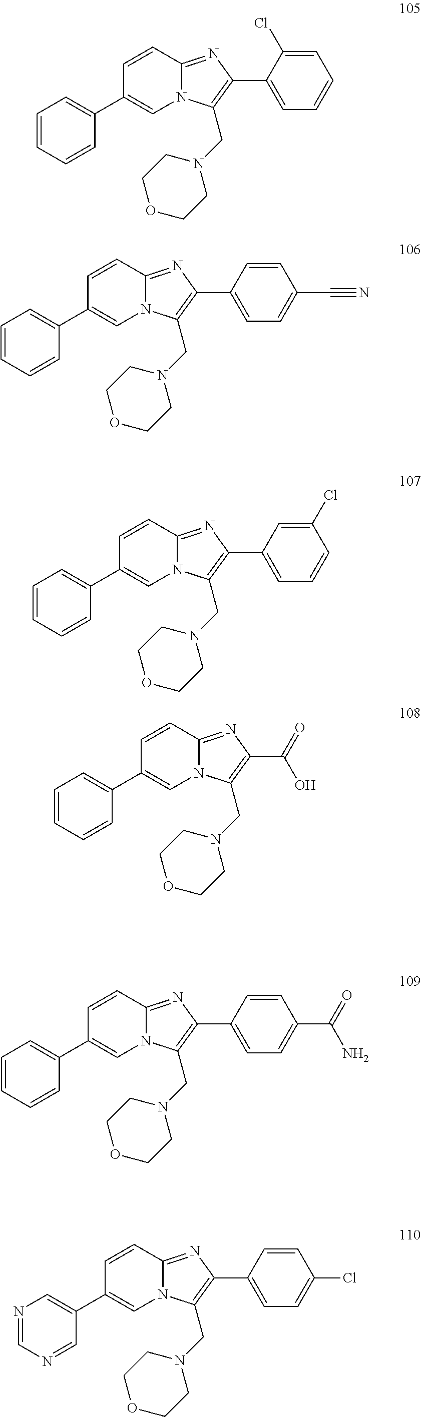

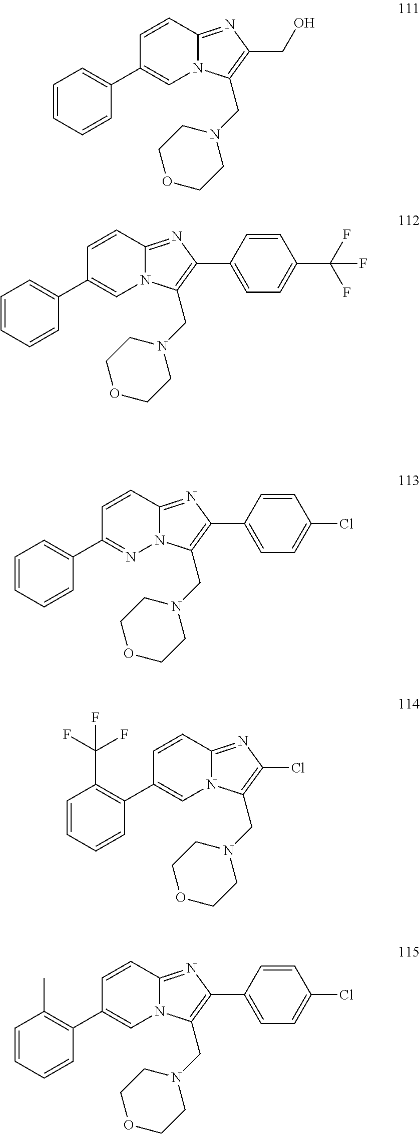 [SMILES: OCc1nc2ccc(-c3ccccc3)cn2c1CN1CCOCC1 ; FC(F)(F)c1ccc(-c2nc3ccc(-c4ccccc4)cn3c2CN2CCOCC2)cc1 ; Clc1ccc(-c2nc3ccc(-c4ccccc4)nn3c2CN2CCOCC2)cc1 ; FC(F)(F)c1ccccc1-c1ccc2nc(Cl)c(CN3CCOCC3)n2c1 ; Cc1ccccc1-c1ccc2nc(-c3ccc(Cl)cc3)c(CN3CCOCC3)n2c1]

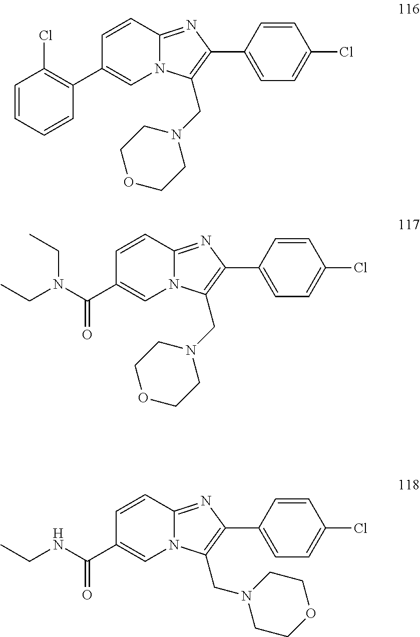 [SMILES: Clc1ccc(-c2nc3ccc(-c4ccccc4Cl)cn3c2CN2CCOCC2)cc1 ; CCN(CC)C(=O)c1ccc2nc(-c3ccc(Cl)cc3)c(CN3CCOCC3)n2c1 ; CCNC(=O)c1ccc2nc(-c3ccc(Cl)cc3)c(CN3CCOCC3)n2c1]

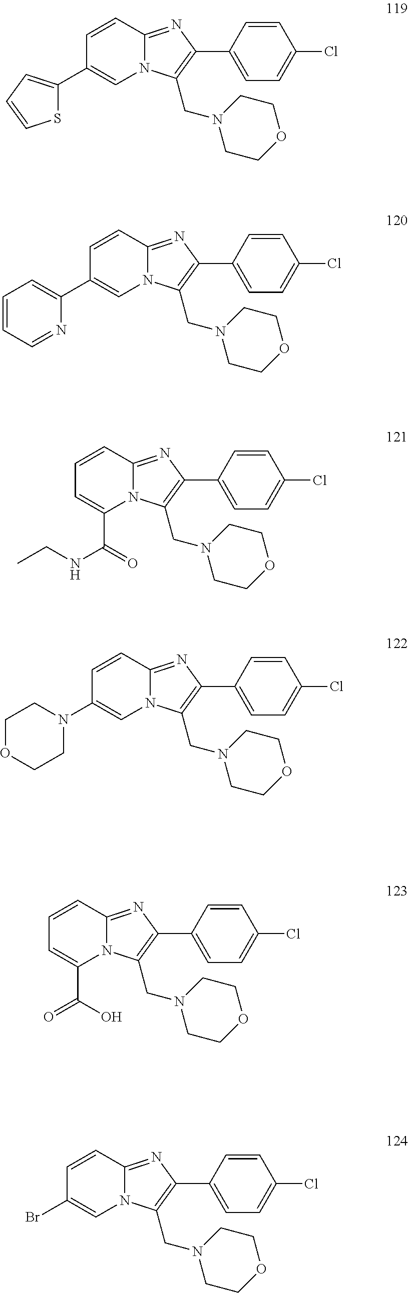 [SMILES: Clc1ccc(-c2nc3ccc(-c4cccs4)cn3c2CN2CCOCC2)cc1 ; Clc1ccc(-c2nc3ccc(-c4ccccn4)cn3c2CN2CCOCC2)cc1 ; CCNC(=O)c1cccc2nc(-c3ccc(Cl)cc3)c(CN3CCOCC3)n12 ; Clc1ccc(-c2nc3ccc(N4CCOCC4)cn3c2CN2CCOCC2)cc1 ; O=C(O)c1cccc2nc(-c3ccc(Cl)cc3)c(CN3CCOCC3)n12 ; Clc1ccc(-c2nc3ccc(Br)cn3c2CN2CCOCC2)cc1]

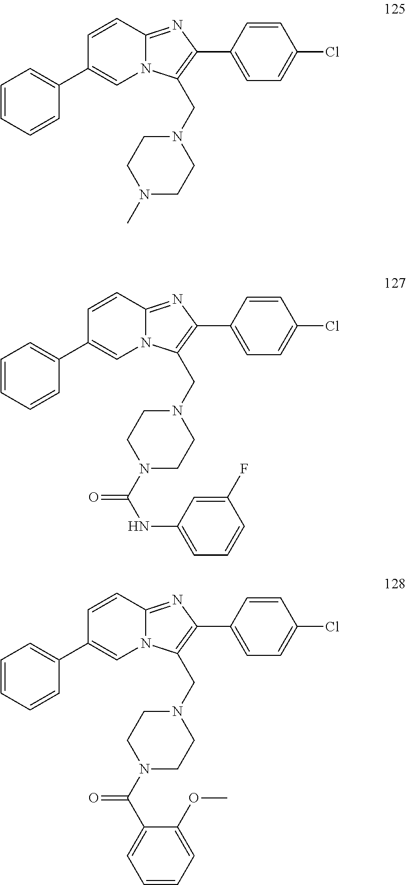 [SMILES: CN1CCN(Cc2c(-c3ccc(Cl)cc3)nc3ccc(-c4ccccc4)cn23)CC1 ; O=C(Nc1cccc(F)c1)N1CCN(Cc2c(-c3ccc(Cl)cc3)nc3ccc(-c4ccccc4)cn23)CC1 ; COc1ccccc1C(=O)N1CCN(Cc2c(-c3ccc(Cl)cc3)nc3ccc(-c4ccccc4)cn23)CC1]

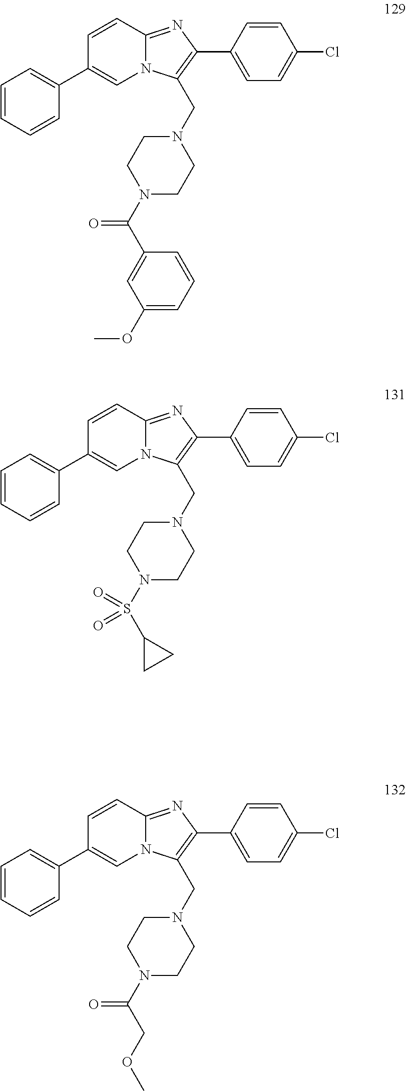 [SMILES: COc1cccc(C(=O)N2CCN(Cc3c(-c4ccc(Cl)cc4)nc4ccc(-c5ccccc5)cn34)CC2)c1 ; O=S(=O)(C1CC1)N1CCN(Cc2c(-c3ccc(Cl)cc3)nc3ccc(-c4ccccc4)cn23)CC1 ; COCC(=O)N1CCN(Cc2c(-c3ccc(Cl)cc3)nc3ccc(-c4ccccc4)cn23)CC1]

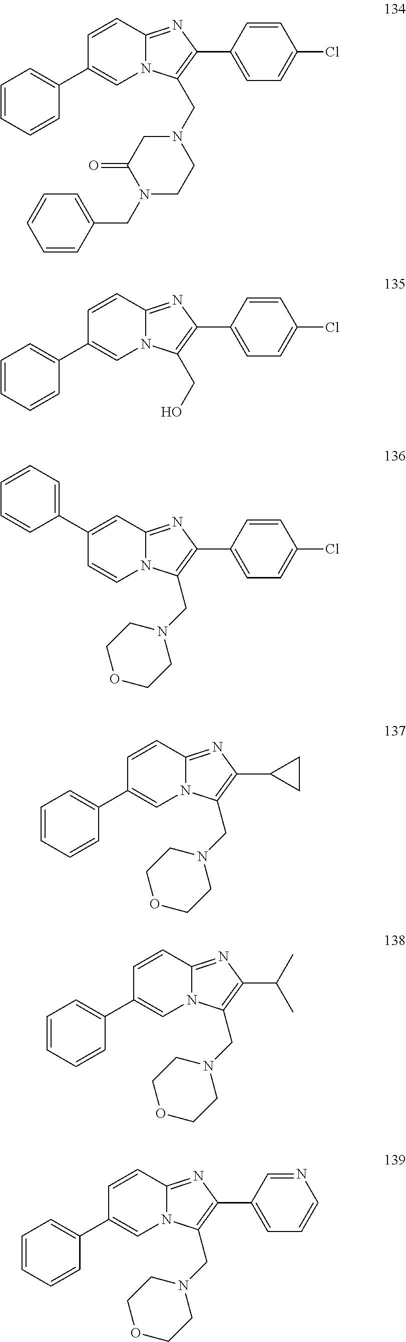

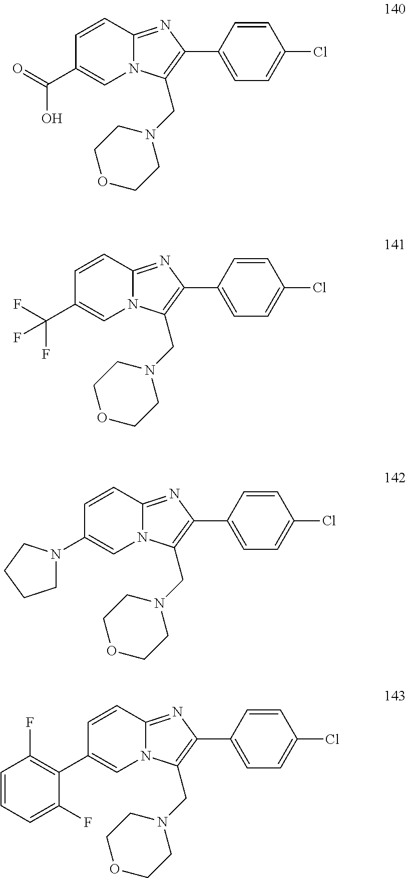 [SMILES: O=C(O)c1ccc2nc(-c3ccc(Cl)cc3)c(CN3CCOCC3)n2c1 ; FC(F)(F)c1ccc2nc(-c3ccc(Cl)cc3)c(CN3CCOCC3)n2c1 ; Clc1ccc(-c2nc3ccc(N4CCCC4)cn3c2CN2CCOCC2)cc1 ; Fc1cccc(F)c1-c1ccc2nc(-c3ccc(Cl)cc3)c(CN3CCOCC3)n2c1]

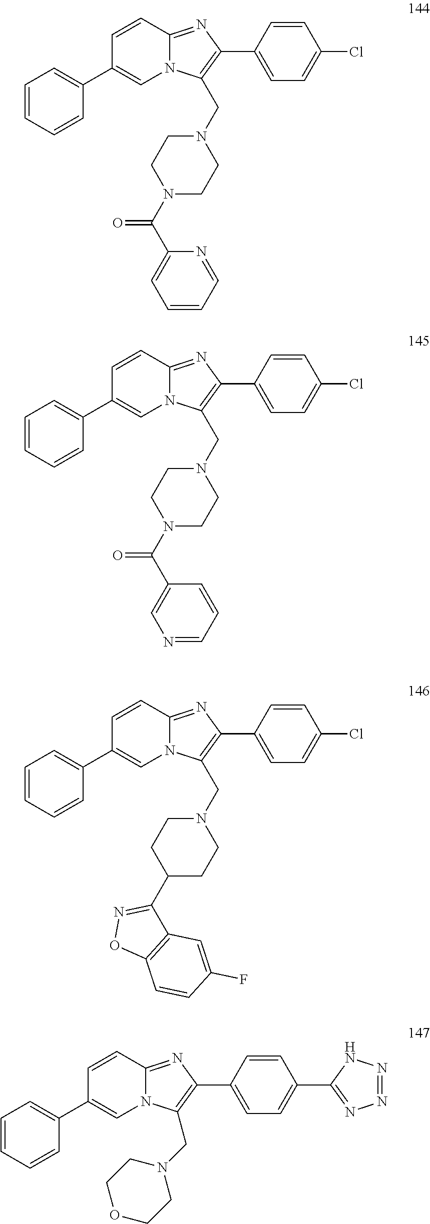 [SMILES: O=C(c1ccccn1)N1CCN(Cc2c(-c3ccc(Cl)cc3)nc3ccc(-c4ccccc4)cn23)CC1 ; O=C(c1cccnc1)N1CCN(Cc2c(-c3ccc(Cl)cc3)nc3ccc(-c4ccccc4)cn23)CC1 ; Fc1ccc2onc(C3CCN(Cc4c(-c5ccc(Cl)cc5)nc5ccc(-c6ccccc6)cn45)CC3)c2c1 ; c1ccc(-c2ccc3nc(-c4ccc(-c5nnn[nH]5)cc4)c(CN4CCOCC4)n3c2)cc1]

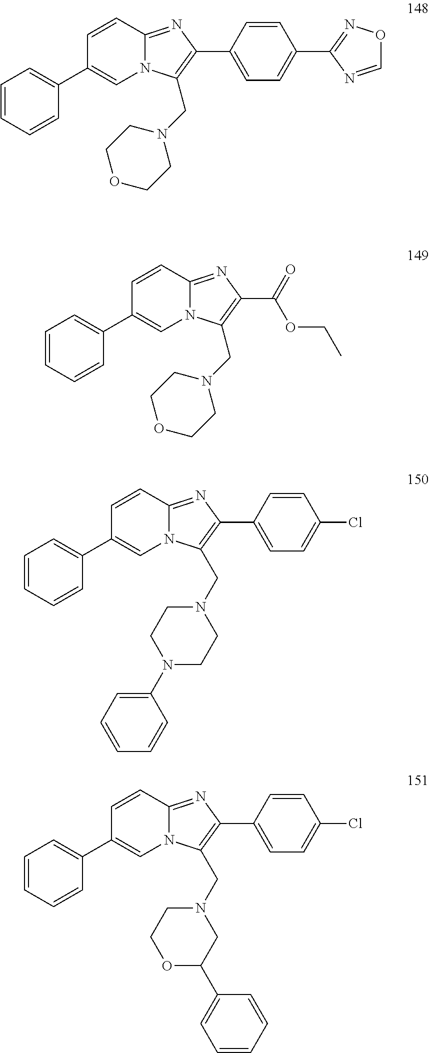

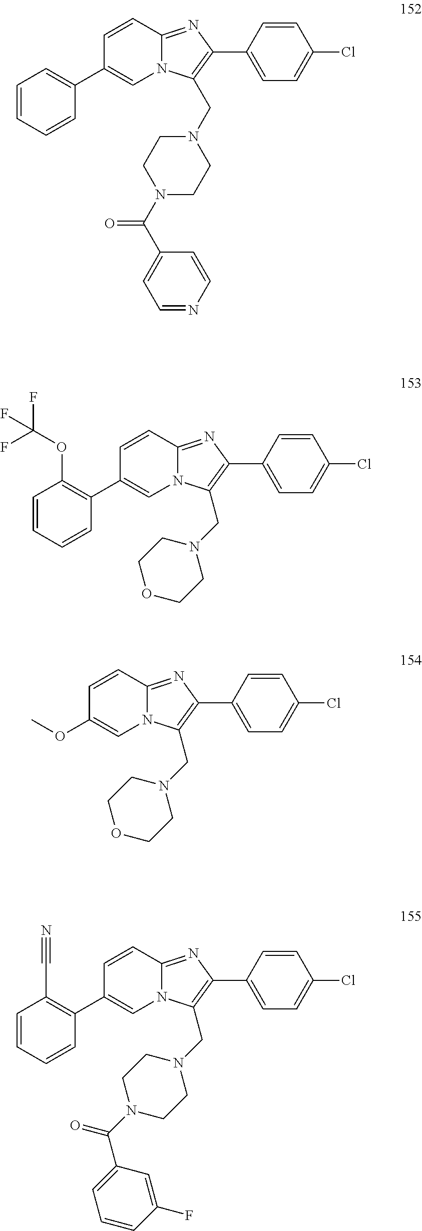 [SMILES: O=C(c1ccncc1)N1CCN(Cc2c(-c3ccc(Cl)cc3)nc3ccc(-c4ccccc4)cn23)CC1 ; FC(F)(F)Oc1ccccc1-c1ccc2nc(-c3ccc(Cl)cc3)c(CN3CCOCC3)n2c1 ; COc1ccc2nc(-c3ccc(Cl)cc3)c(CN3CCOCC3)n2c1 ; N#Cc1ccccc1-c1ccc2nc(-c3ccc(Cl)cc3)c(CN3CCN(C(=O)c4cccc(F)c4)CC3)n2c1]

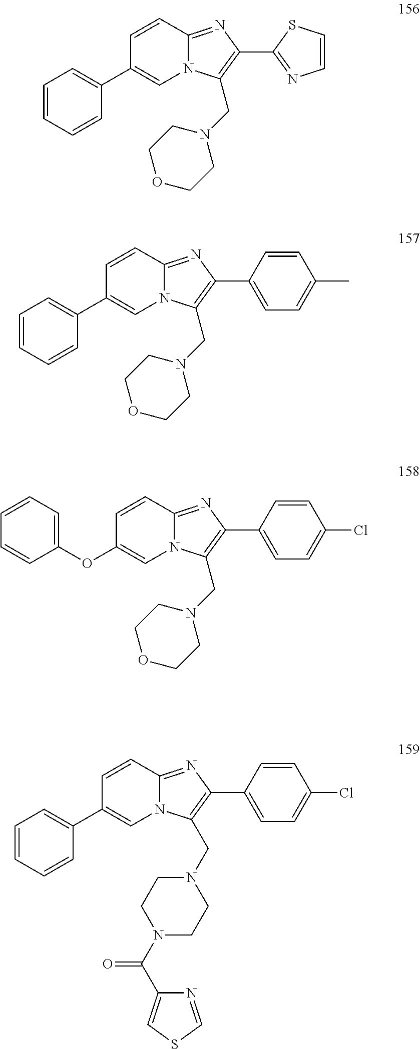 [SMILES: c1ccc(-c2ccc3nc(-c4nccs4)c(CN4CCOCC4)n3c2)cc1 ; Cc1ccc(-c2nc3ccc(-c4ccccc4)cn3c2CN2CCOCC2)cc1 ; Clc1ccc(-c2nc3ccc(Oc4ccccc4)cn3c2CN2CCOCC2)cc1 ; O=C(c1cscn1)N1CCN(Cc2c(-c3ccc(Cl)cc3)nc3ccc(-c4ccccc4)cn23)CC1]

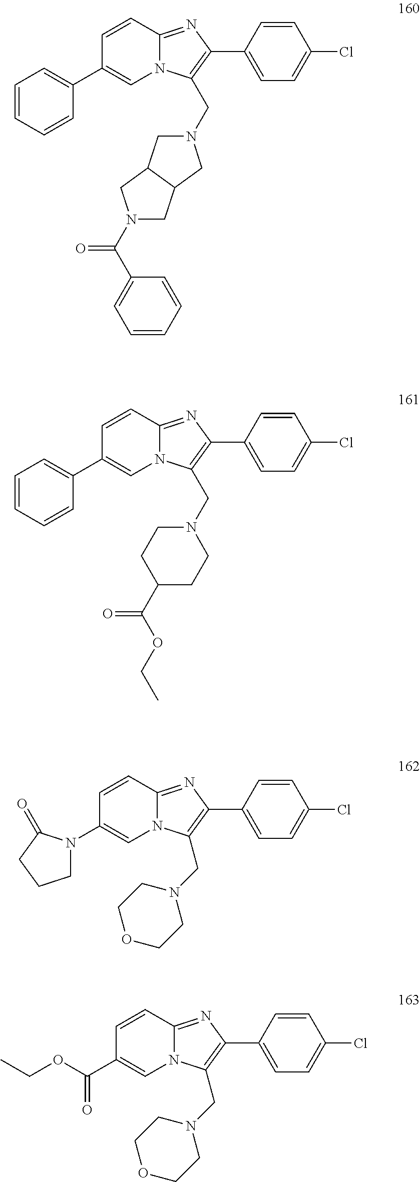 [SMILES: O=C(c1ccccc1)N1CC2CN(Cc3c(-c4ccc(Cl)cc4)nc4ccc(-c5ccccc5)cn34)CC2C1 ; CCOC(=O)C1CCN(Cc2c(-c3ccc(Cl)cc3)nc3ccc(-c4ccccc4)cn23)CC1 ; O=C1CCCN1c1ccc2nc(-c3ccc(Cl)cc3)c(CN3CCOCC3)n2c1 ; CCOC(=O)c1ccc2nc(-c3ccc(Cl)cc3)c(CN3CCOCC3)n2c1]

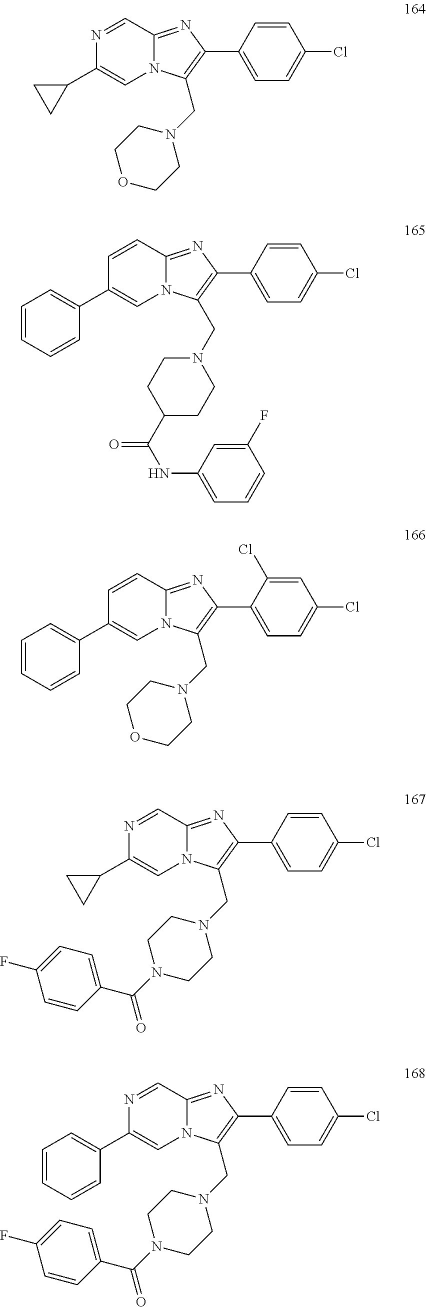 [SMILES: Clc1ccc(-c2nc3cnc(C4CC4)cn3c2CN2CCOCC2)cc1 ; O=C(Nc1cccc(F)c1)C1CCN(Cc2c(-c3ccc(Cl)cc3)nc3ccc(-c4ccccc4)cn23)CC1 ; Clc1ccc(-c2nc3ccc(-c4ccccc4)cn3c2CN2CCOCC2)c(Cl)c1 ; O=C(c1ccc(F)cc1)N1CCN(Cc2c(-c3ccc(Cl)cc3)nc3cnc(C4CC4)cn23)CC1 ; O=C(c1ccc(F)cc1)N1CCN(Cc2c(-c3ccc(Cl)cc3)nc3cnc(-c4ccccc4)cn23)CC1]

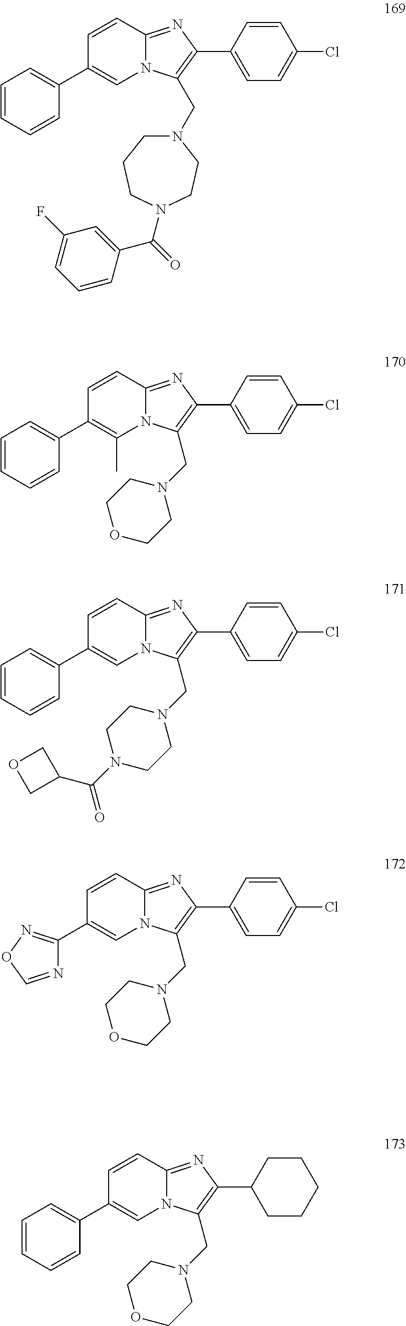 [SMILES: O=C(c1cccc(F)c1)N1CCCN(Cc2c(-c3ccc(Cl)cc3)nc3ccc(-c4ccccc4)cn23)CC1 ; Cc1c(-c2ccccc2)ccc2nc(-c3ccc(Cl)cc3)c(CN3CCOCC3)n12 ; O=C(C1COC1)N1CCN(Cc2c(-c3ccc(Cl)cc3)nc3ccc(-c4ccccc4)cn23)CC1 ; Clc1ccc(-c2nc3ccc(-c4ncon4)cn3c2CN2CCOCC2)cc1 ; c1ccc(-c2ccc3nc(C4CCCCC4)c(CN4CCOCC4)n3c2)cc1]

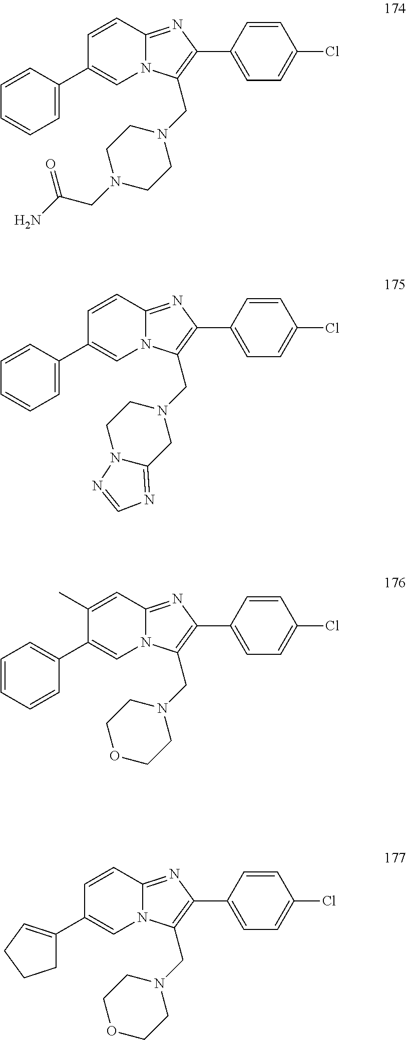 [SMILES: NC(=O)CN1CCN(Cc2c(-c3ccc(Cl)cc3)nc3ccc(-c4ccccc4)cn23)CC1 ; Clc1ccc(-c2nc3ccc(-c4ccccc4)cn3c2CN2CCn3ncnc3C2)cc1 ; Cc1cc2nc(-c3ccc(Cl)cc3)c(CN3CCOCC3)n2cc1-c1ccccc1 ; Clc1ccc(-c2nc3ccc(C4=CCCC4)cn3c2CN2CCOCC2)cc1]

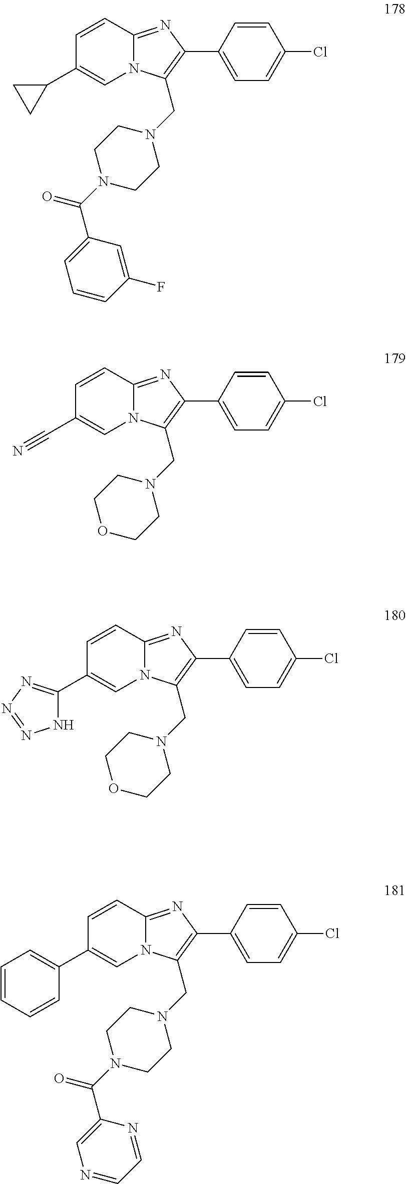 [SMILES: O=C(c1cccc(F)c1)N1CCN(Cc2c(-c3ccc(Cl)cc3)nc3ccc(C4CC4)cn23)CC1 ; N#Cc1ccc2nc(-c3ccc(Cl)cc3)c(CN3CCOCC3)n2c1 ; Clc1ccc(-c2nc3ccc(-c4nnn[nH]4)cn3c2CN2CCOCC2)cc1 ; O=C(c1cnccn1)N1CCN(Cc2c(-c3ccc(Cl)cc3)nc3ccc(-c4ccccc4)cn23)CC1]

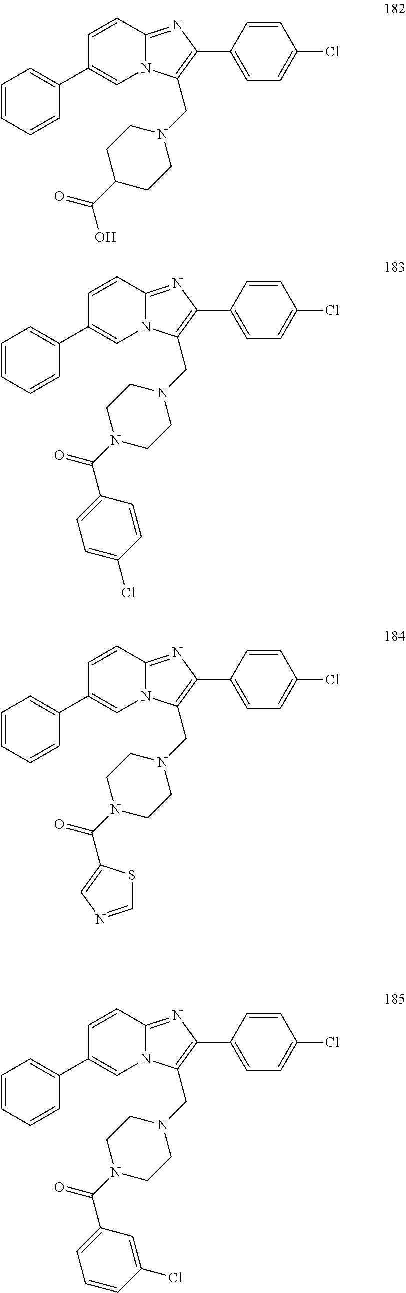

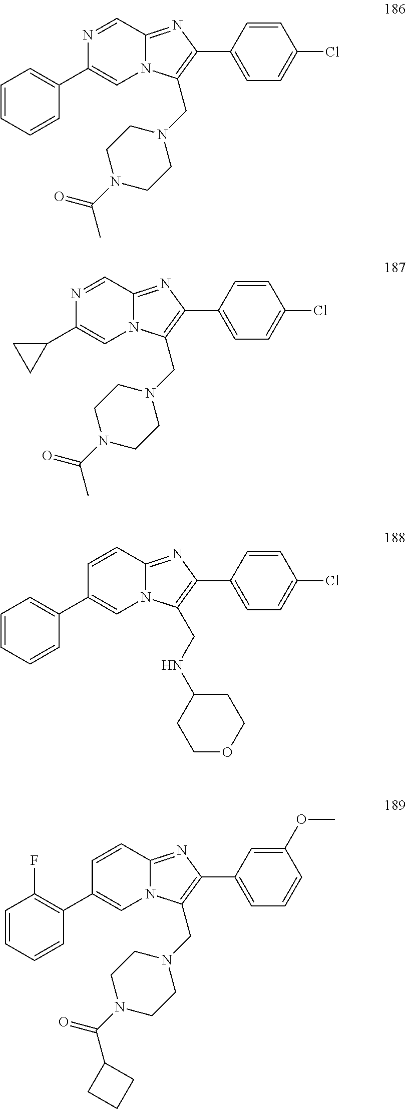

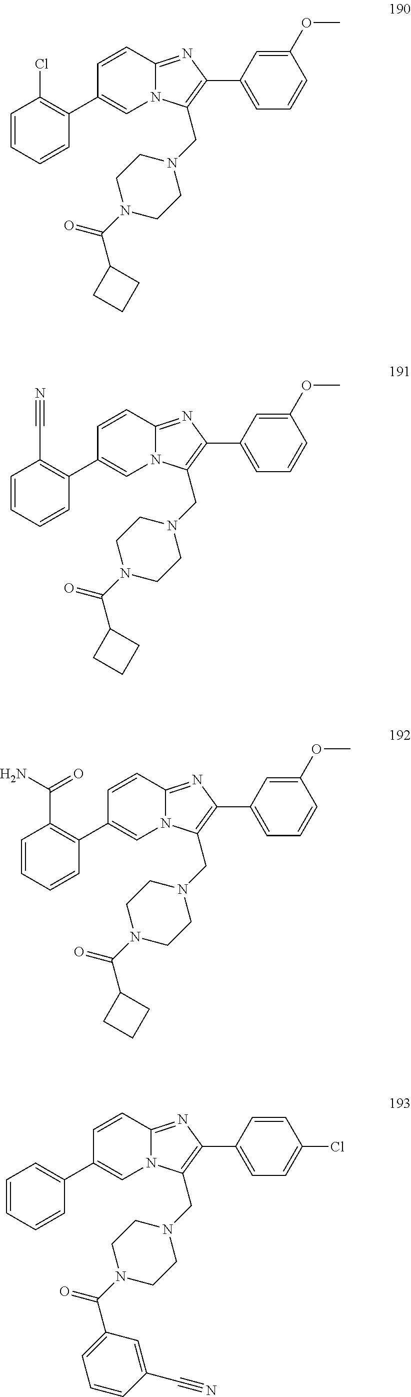 [SMILES: COc1cccc(-c2nc3ccc(-c4ccccc4Cl)cn3c2CN2CCN(C(=O)C3CCC3)CC2)c1 ; COc1cccc(-c2nc3ccc(-c4ccccc4C#N)cn3c2CN2CCN(C(=O)C3CCC3)CC2)c1 ; COc1cccc(-c2nc3ccc(-c4ccccc4C(N)=O)cn3c2CN2CCN(C(=O)C3CCC3)CC2)c1 ; N#Cc1cccc(C(=O)N2CCN(Cc3c(-c4ccc(Cl)cc4)nc4ccc(-c5ccccc5)cn34)CC2)c1]

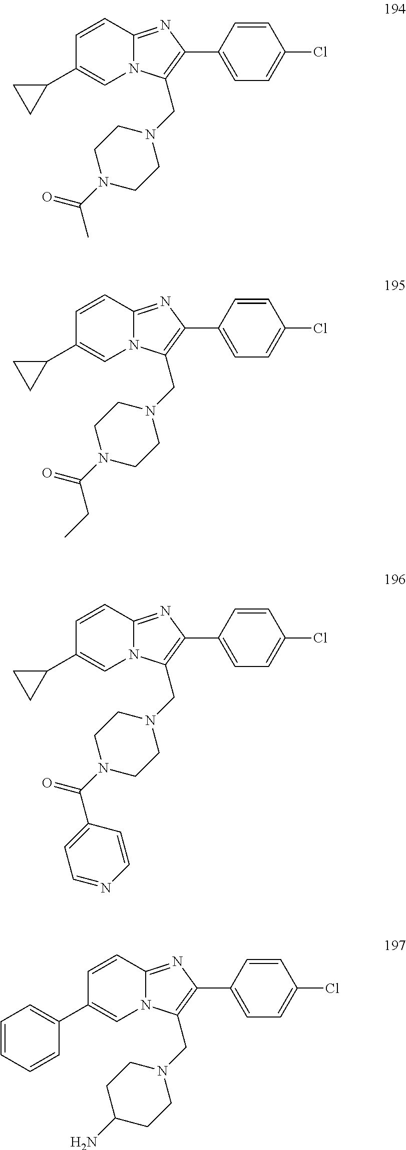 [SMILES: CC(=O)N1CCN(Cc2c(-c3ccc(Cl)cc3)nc3ccc(C4CC4)cn23)CC1 ; CCC(=O)N1CCN(Cc2c(-c3ccc(Cl)cc3)nc3ccc(C4CC4)cn23)CC1 ; O=C(c1ccncc1)N1CCN(Cc2c(-c3ccc(Cl)cc3)nc3ccc(C4CC4)cn23)CC1 ; NC1CCN(Cc2c(-c3ccc(Cl)cc3)nc3ccc(-c4ccccc4)cn23)CC1]

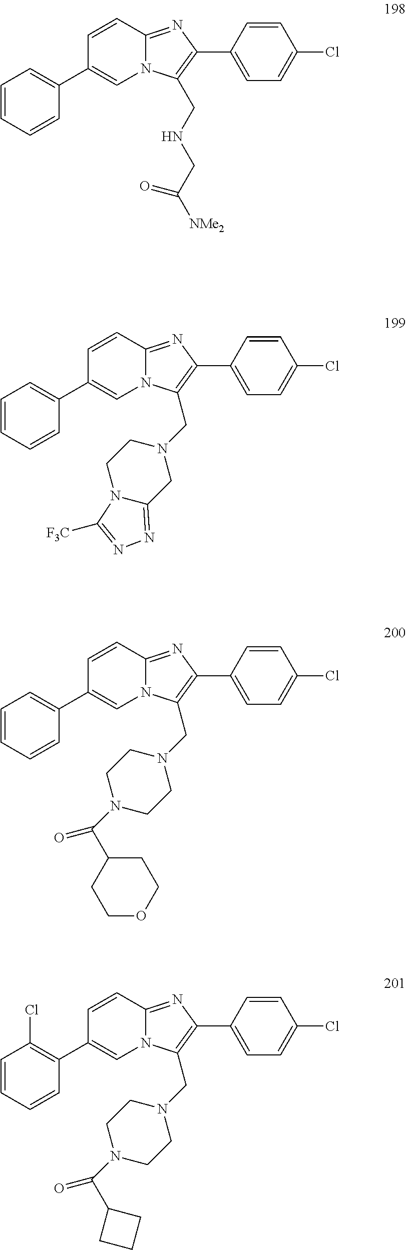 [SMILES: CN(C)C(=O)CNCc1c(-c2ccc(Cl)cc2)nc2ccc(-c3ccccc3)cn12 ; FC(F)(F)c1nnc2n1CCN(Cc1c(-c3ccc(Cl)cc3)nc3ccc(-c4ccccc4)cn13)C2 ; O=C(C1CCOCC1)N1CCN(Cc2c(-c3ccc(Cl)cc3)nc3ccc(-c4ccccc4)cn23)CC1 ; O=C(C1CCC1)N1CCN(Cc2c(-c3ccc(Cl)cc3)nc3ccc(-c4ccccc4Cl)cn23)CC1]

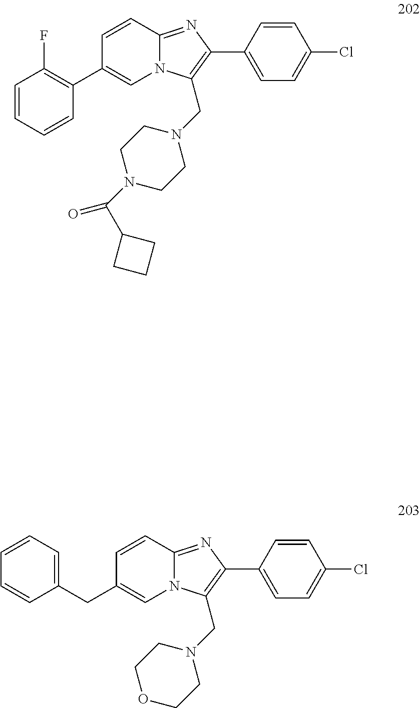 [SMILES: O=C(C1CCC1)N1CCN(Cc2c(-c3ccc(Cl)cc3)nc3ccc(-c4ccccc4F)cn23)CC1 ; Clc1ccc(-c2nc3ccc(Cc4ccccc4)cn3c2CN2CCOCC2)cc1]

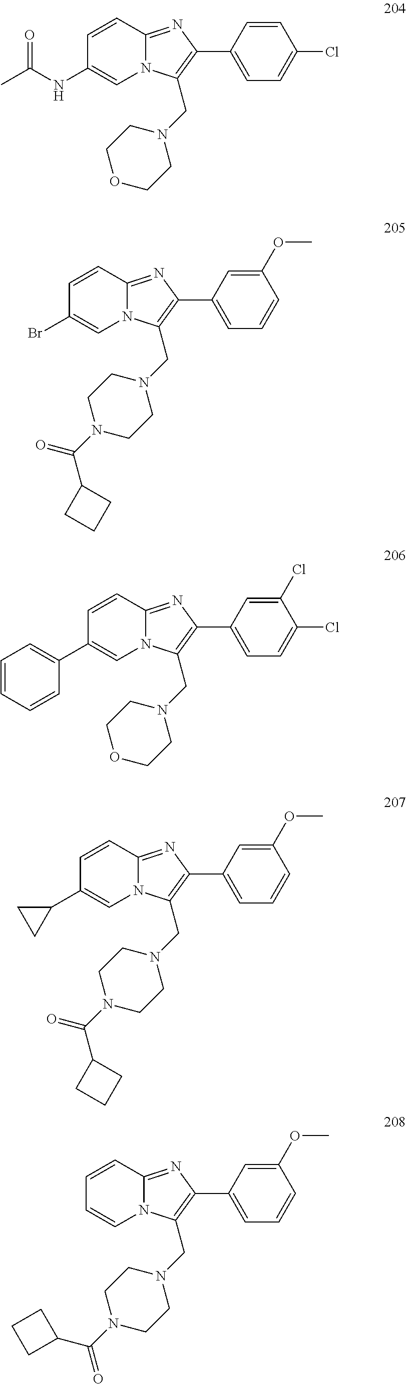 [SMILES: CC(=O)Nc1ccc2nc(-c3ccc(Cl)cc3)c(CN3CCOCC3)n2c1 ; COc1cccc(-c2nc3ccc(Br)cn3c2CN2CCN(C(=O)C3CCC3)CC2)c1 ; Clc1ccc(-c2nc3ccc(-c4ccccc4)cn3c2CN2CCOCC2)cc1Cl ; COc1cccc(-c2nc3ccc(C4CC4)cn3c2CN2CCN(C(=O)C3CCC3)CC2)c1 ; COc1cccc(-c2nc3ccccn3c2CN2CCN(C(=O)C3CCC3)CC2)c1]

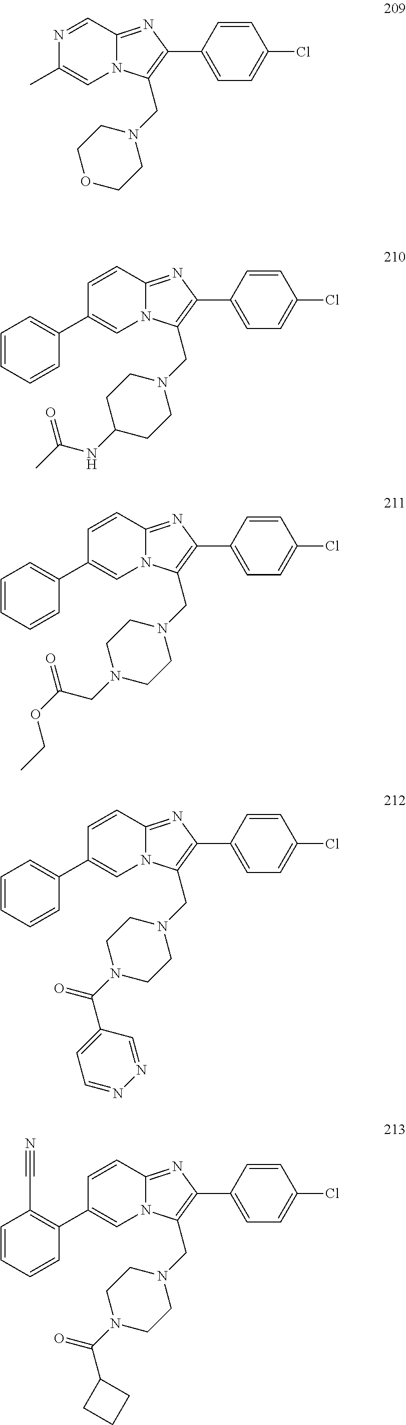 [SMILES: Cc1cn2c(CN3CCOCC3)c(-c3ccc(Cl)cc3)nc2cn1 ; CC(=O)NC1CCN(Cc2c(-c3ccc(Cl)cc3)nc3ccc(-c4ccccc4)cn23)CC1 ; CCOC(=O)CN1CCN(Cc2c(-c3ccc(Cl)cc3)nc3ccc(-c4ccccc4)cn23)CC1 ; O=C(c1ccnnc1)N1CCN(Cc2c(-c3ccc(Cl)cc3)nc3ccc(-c4ccccc4)cn23)CC1 ; N#Cc1ccccc1-c1ccc2nc(-c3ccc(Cl)cc3)c(CN3CCN(C(=O)C4CCC4)CC3)n2c1]

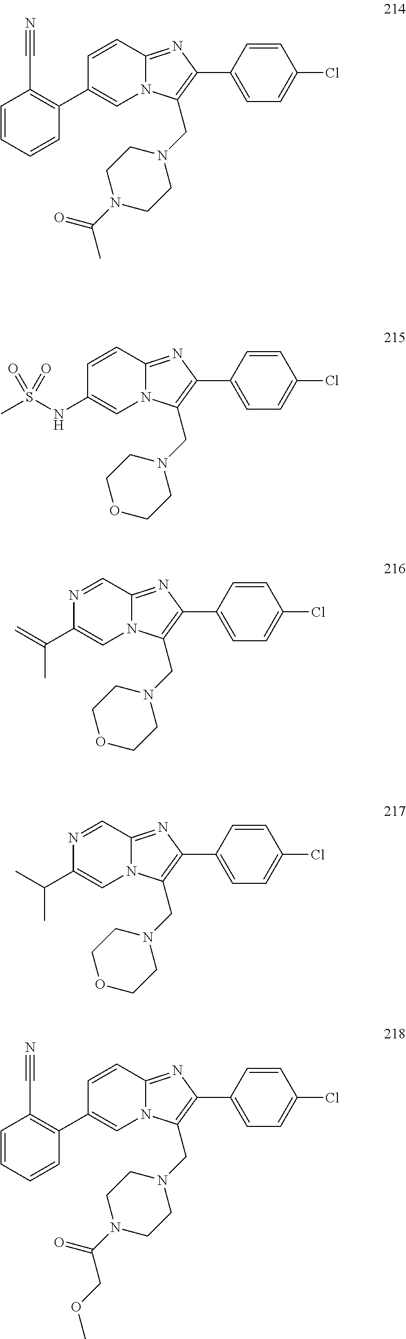 [SMILES: CC(=O)N1CCN(Cc2c(-c3ccc(Cl)cc3)nc3ccc(-c4ccccc4C#N)cn23)CC1 ; CS(=O)(=O)Nc1ccc2nc(-c3ccc(Cl)cc3)c(CN3CCOCC3)n2c1 ; C=C(C)c1cn2c(CN3CCOCC3)c(-c3ccc(Cl)cc3)nc2cn1 ; CC(C)c1cn2c(CN3CCOCC3)c(-c3ccc(Cl)cc3)nc2cn1 ; COCC(=O)N1CCN(Cc2c(-c3ccc(Cl)cc3)nc3ccc(-c4ccccc4C#N)cn23)CC1]

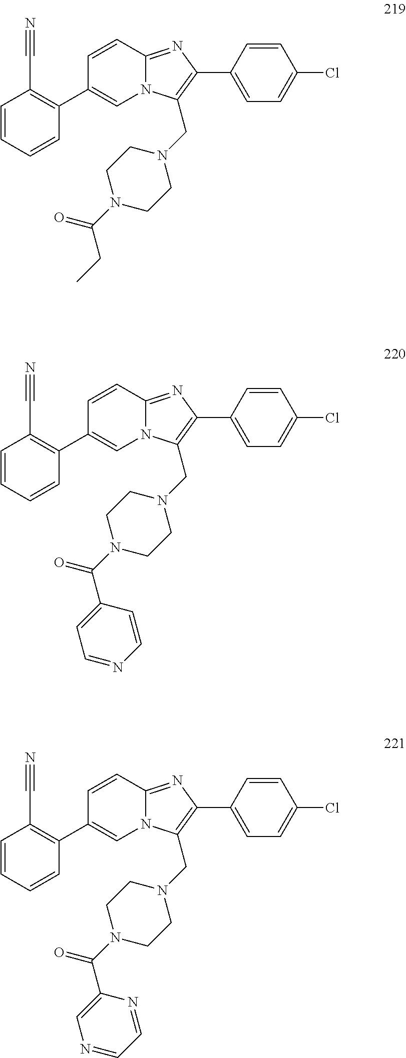 [SMILES: CCC(=O)N1CCN(Cc2c(-c3ccc(Cl)cc3)nc3ccc(-c4ccccc4C#N)cn23)CC1 ; N#Cc1ccccc1-c1ccc2nc(-c3ccc(Cl)cc3)c(CN3CCN(C(=O)c4ccncc4)CC3)n2c1 ; N#Cc1ccccc1-c1ccc2nc(-c3ccc(Cl)cc3)c(CN3CCN(C(=O)c4cnccn4)CC3)n2c1]

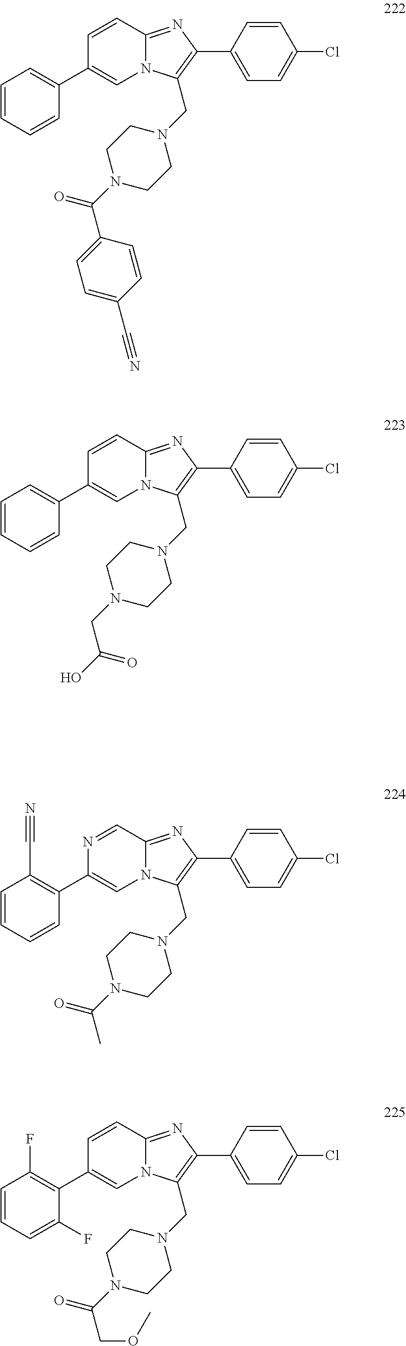 [SMILES: N#Cc1ccc(C(=O)N2CCN(Cc3c(-c4ccc(Cl)cc4)nc4ccc(-c5ccccc5)cn34)CC2)cc1 ; O=C(O)CN1CCN(Cc2c(-c3ccc(Cl)cc3)nc3ccc(-c4ccccc4)cn23)CC1 ; CC(=O)N1CCN(Cc2c(-c3ccc(Cl)cc3)nc3cnc(-c4ccccc4C#N)cn23)CC1 ; COCC(=O)N1CCN(Cc2c(-c3ccc(Cl)cc3)nc3ccc(-c4c(F)cccc4F)cn23)CC1]

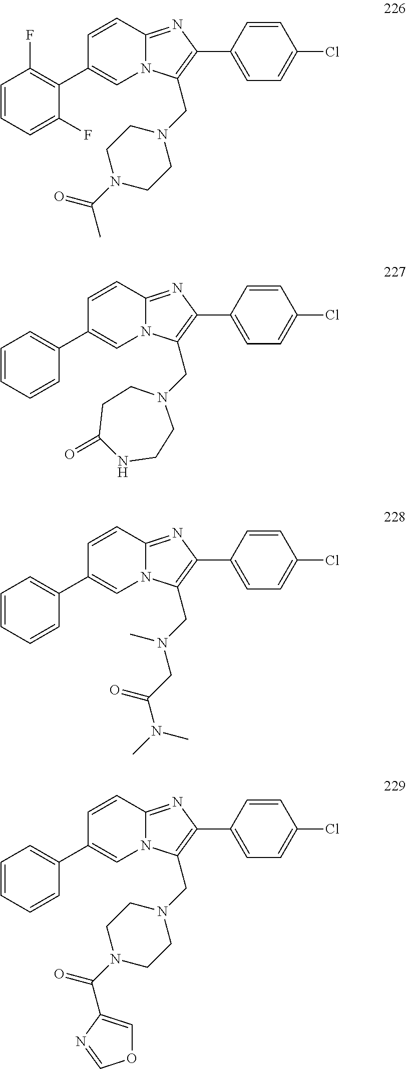

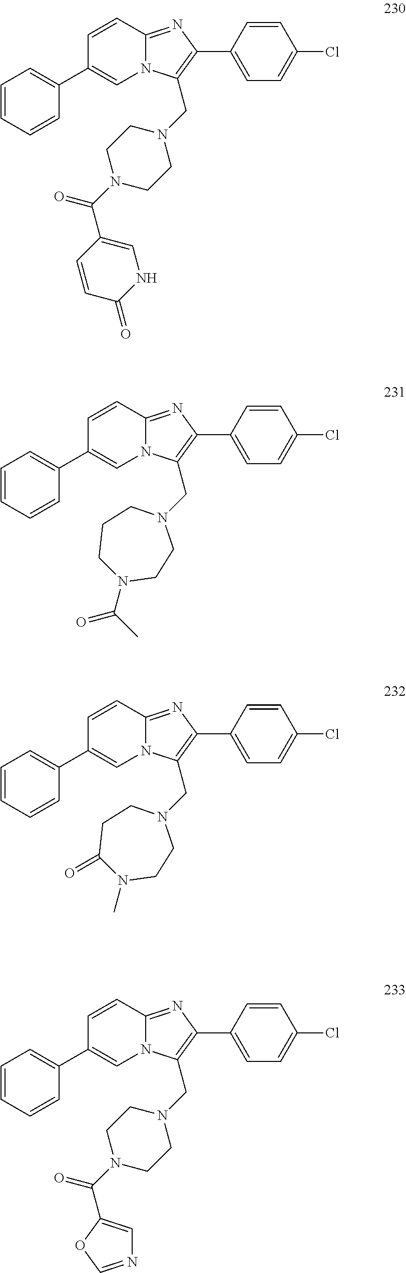 [SMILES: O=C(c1ccc(=O)[nH]c1)N1CCN(Cc2c(-c3ccc(Cl)cc3)nc3ccc(-c4ccccc4)cn23)CC1 ; CC(=O)N1CCCN(Cc2c(-c3ccc(Cl)cc3)nc3ccc(-c4ccccc4)cn23)CC1 ; CN1CCN(Cc2c(-c3ccc(Cl)cc3)nc3ccc(-c4ccccc4)cn23)CCC1=O ; O=C(c1cnco1)N1CCN(Cc2c(-c3ccc(Cl)cc3)nc3ccc(-c4ccccc4)cn23)CC1]

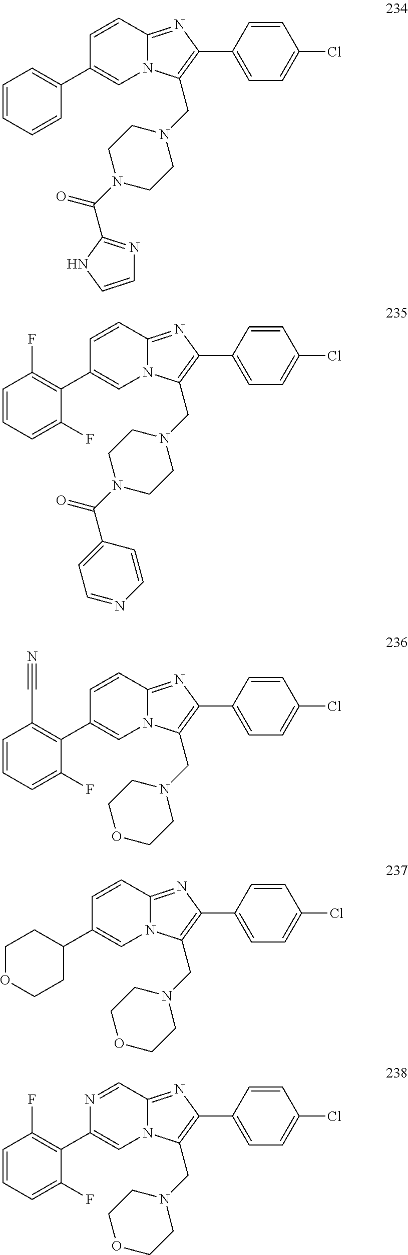

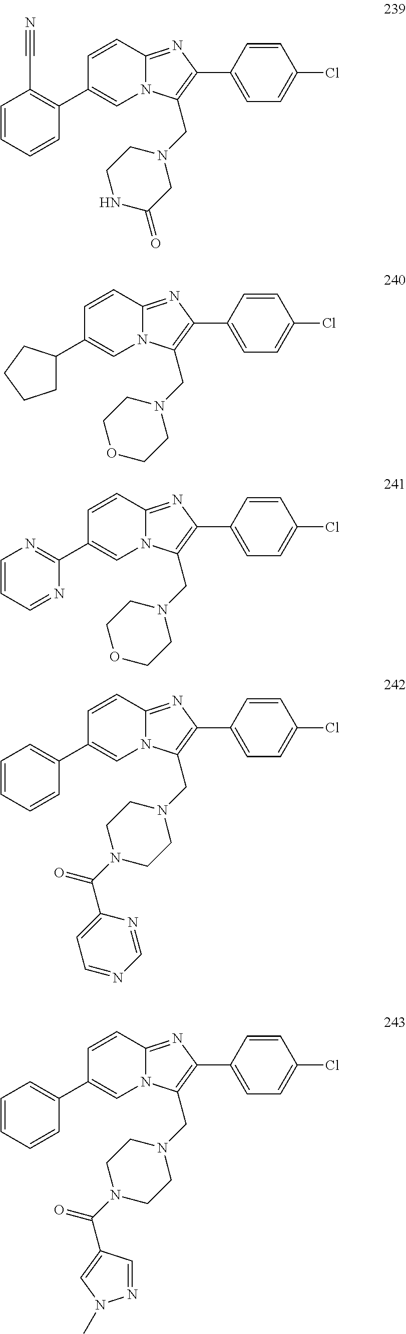

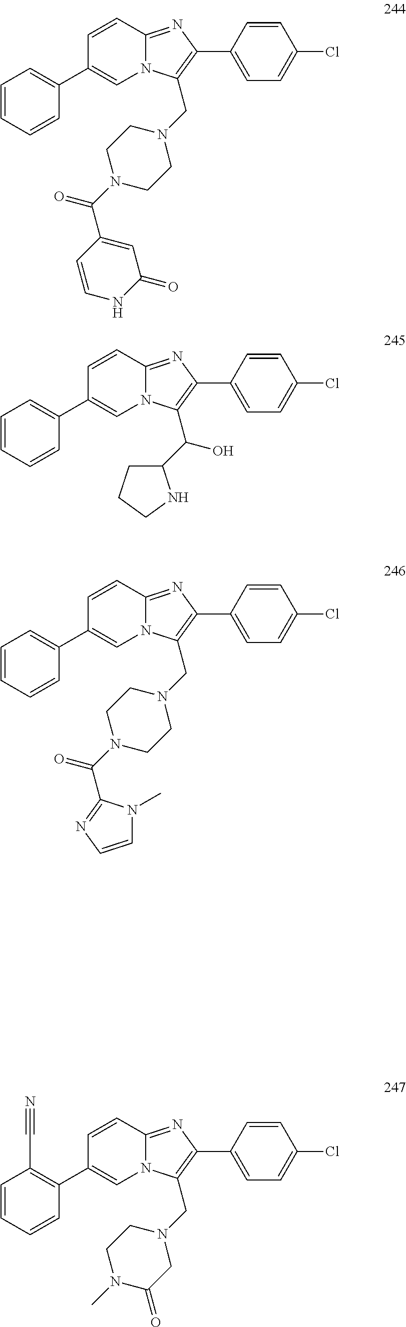 [SMILES: O=C(c1cc[nH]c(=O)c1)N1CCN(Cc2c(-c3ccc(Cl)cc3)nc3ccc(-c4ccccc4)cn23)CC1 ; OC(c1c(-c2ccc(Cl)cc2)nc2ccc(-c3ccccc3)cn12)C1CCCN1 ; Cn1ccnc1C(=O)N1CCN(Cc2c(-c3ccc(Cl)cc3)nc3ccc(-c4ccccc4)cn23)CC1 ; CN1CCN(Cc2c(-c3ccc(Cl)cc3)nc3ccc(-c4ccccc4C#N)cn23)CC1=O]

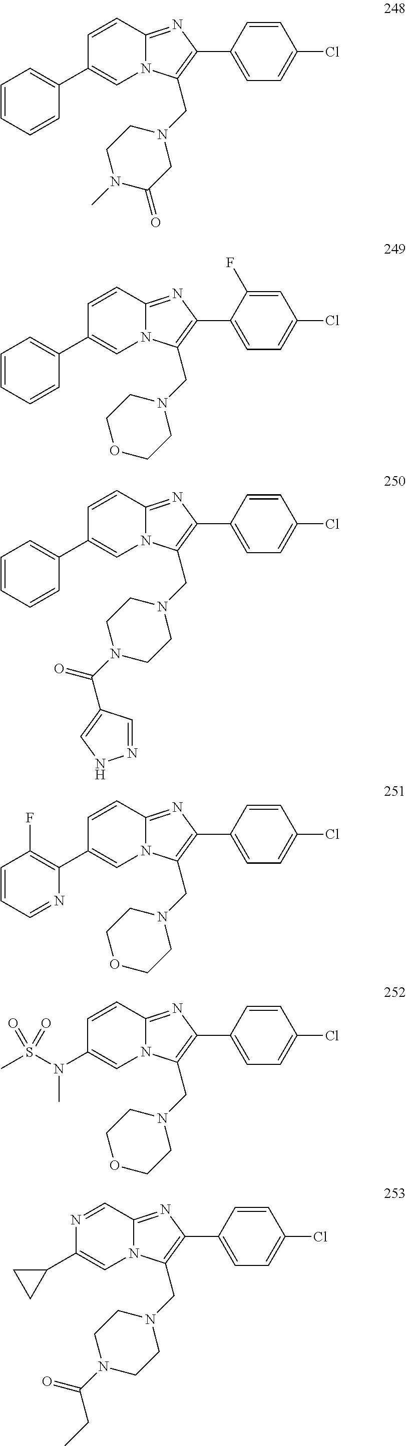

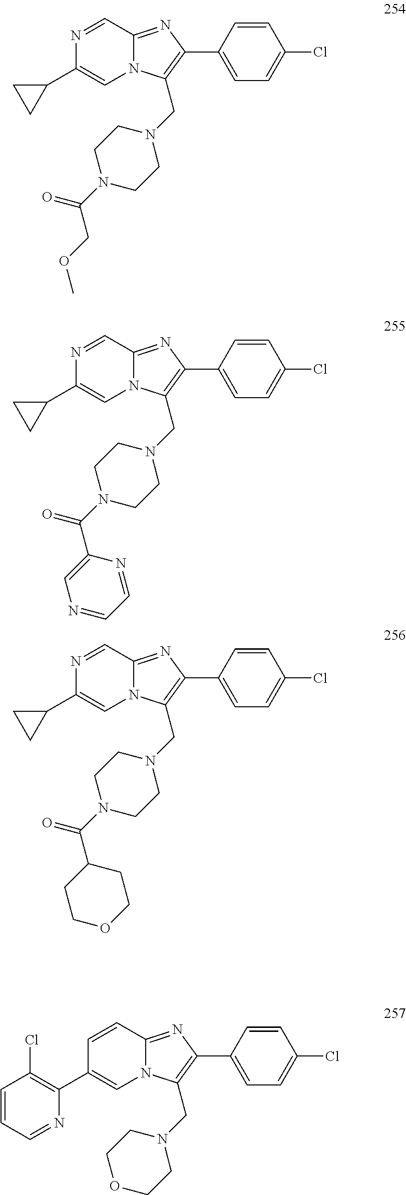

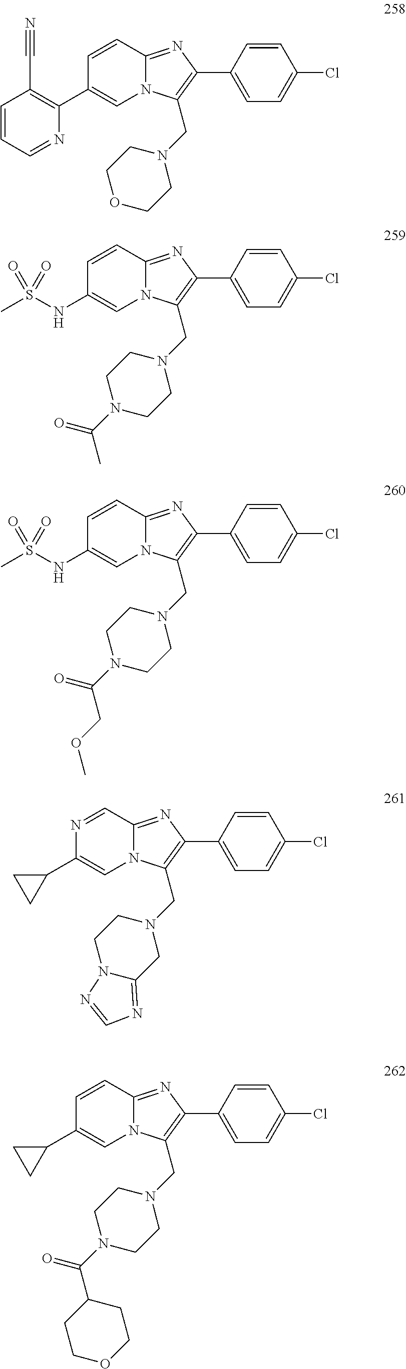

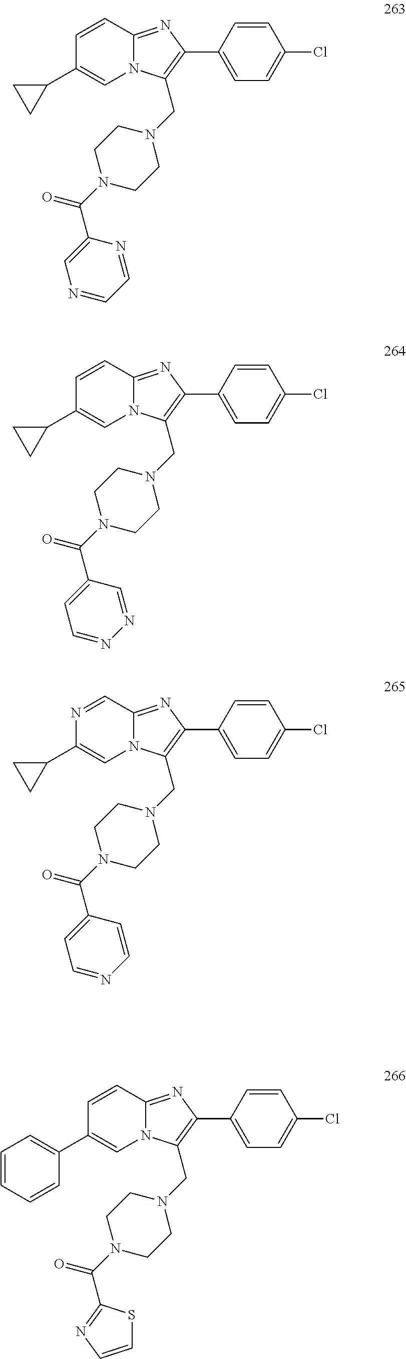 [SMILES: O=C(c1cnccn1)N1CCN(Cc2c(-c3ccc(Cl)cc3)nc3ccc(C4CC4)cn23)CC1 ; O=C(c1ccnnc1)N1CCN(Cc2c(-c3ccc(Cl)cc3)nc3ccc(C4CC4)cn23)CC1 ; O=C(c1ccncc1)N1CCN(Cc2c(-c3ccc(Cl)cc3)nc3cnc(C4CC4)cn23)CC1 ; O=C(c1nccs1)N1CCN(Cc2c(-c3ccc(Cl)cc3)nc3ccc(-c4ccccc4)cn23)CC1]

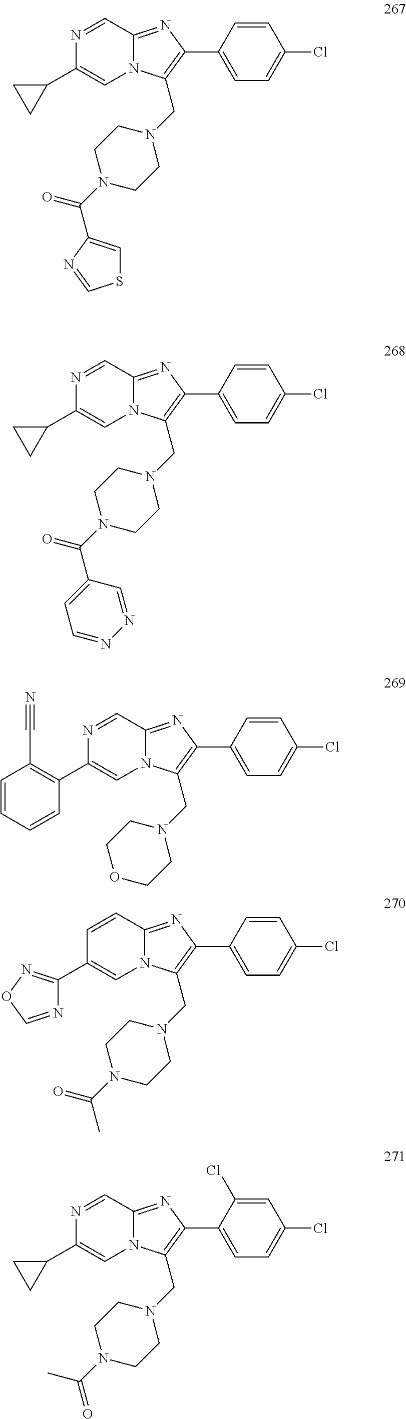

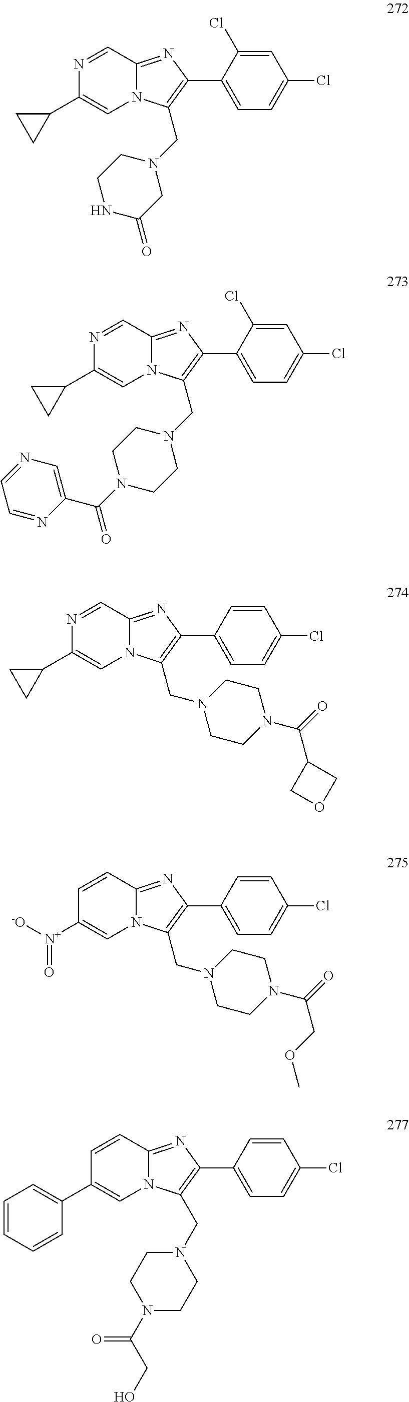

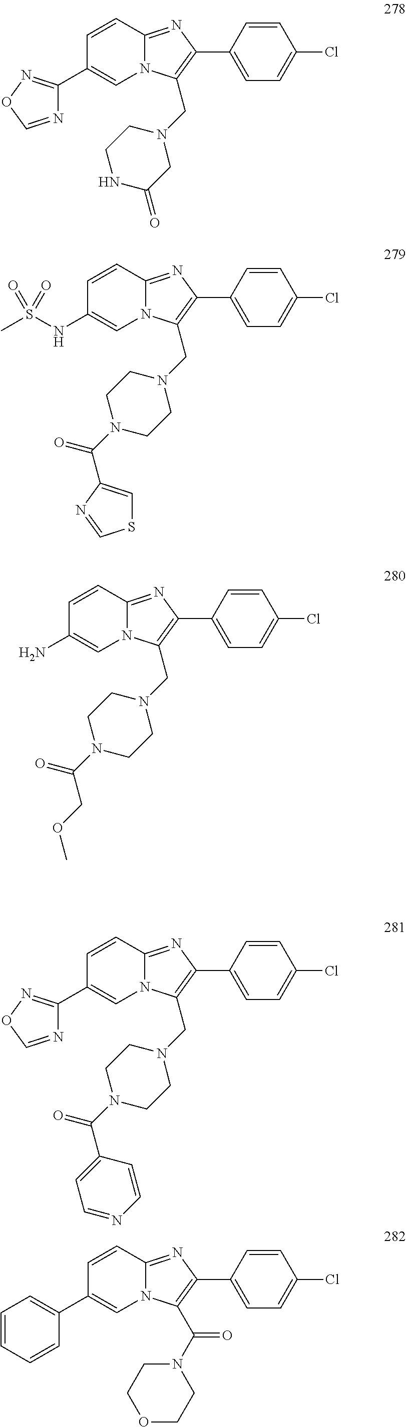

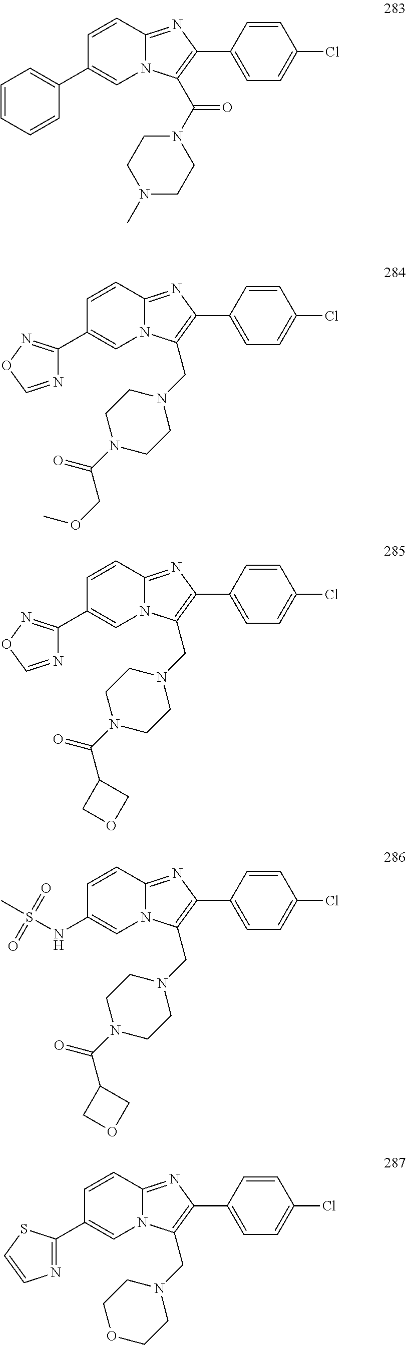

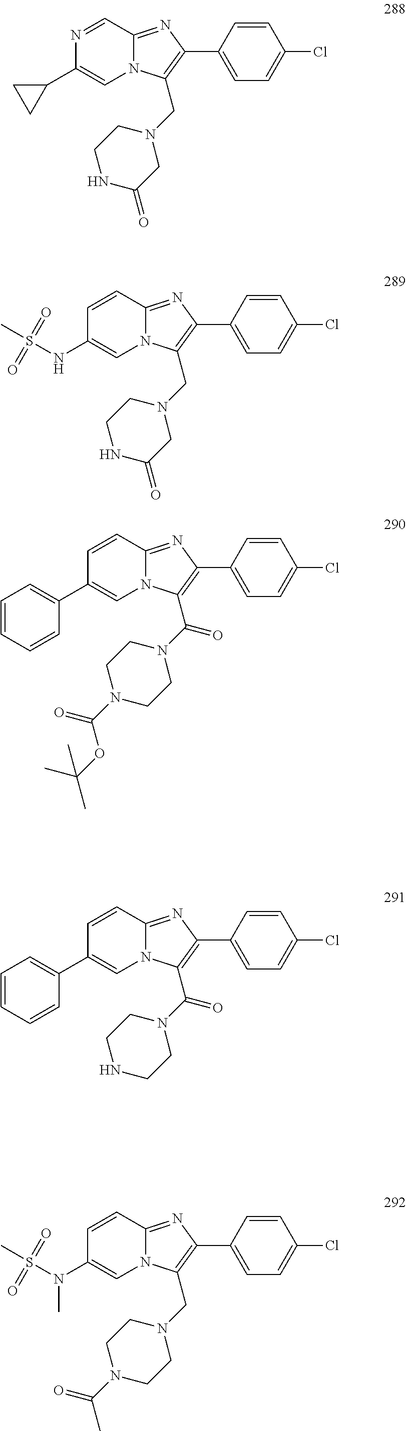 [SMILES: O=C1CN(Cc2c(-c3ccc(Cl)cc3)nc3cnc(C4CC4)cn23)CCN1 ; CS(=O)(=O)Nc1ccc2nc(-c3ccc(Cl)cc3)c(CN3CCNC(=O)C3)n2c1 ; CC(C)(C)OC(=O)N1CCN(C(=O)c2c(-c3ccc(Cl)cc3)nc3ccc(-c4ccccc4)cn23)CC1 ; O=C(c1c(-c2ccc(Cl)cc2)nc2ccc(-c3ccccc3)cn12)N1CCNCC1 ; CC(=O)N1CCN(Cc2c(-c3ccc(Cl)cc3)nc3ccc(N(C)S(C)(=O)=O)cn23)CC1]

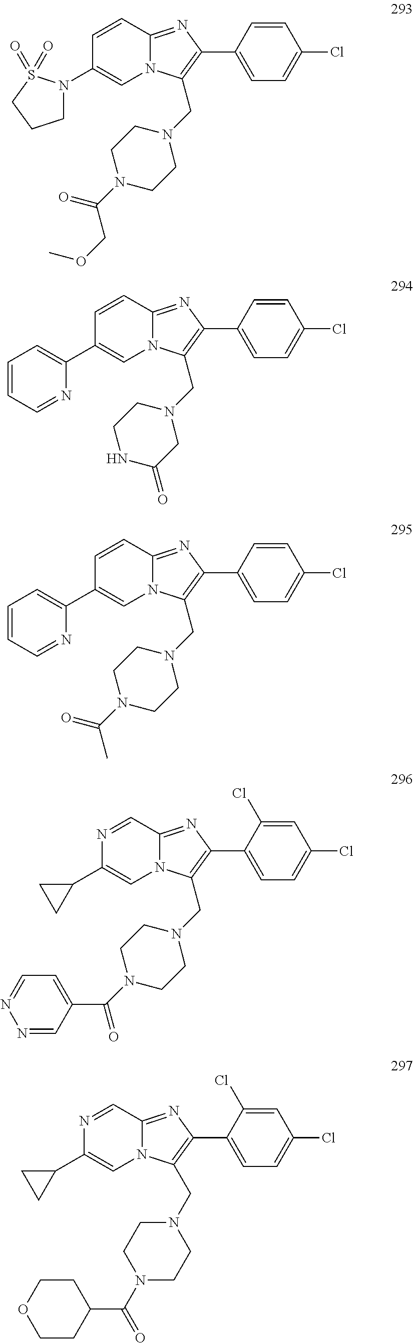 [SMILES: COCC(=O)N1CCN(Cc2c(-c3ccc(Cl)cc3)nc3ccc(N4CCCS4(=O)=O)cn23)CC1 ; O=C1CN(Cc2c(-c3ccc(Cl)cc3)nc3ccc(-c4ccccn4)cn23)CCN1 ; CC(=O)N1CCN(Cc2c(-c3ccc(Cl)cc3)nc3ccc(-c4ccccn4)cn23)CC1 ; O=C(c1ccnnc1)N1CCN(Cc2c(-c3ccc(Cl)cc3Cl)nc3cnc(C4CC4)cn23)CC1 ; O=C(C1CCOCC1)N1CCN(Cc2c(-c3ccc(Cl)cc3Cl)nc3cnc(C4CC4)cn23)CC1]

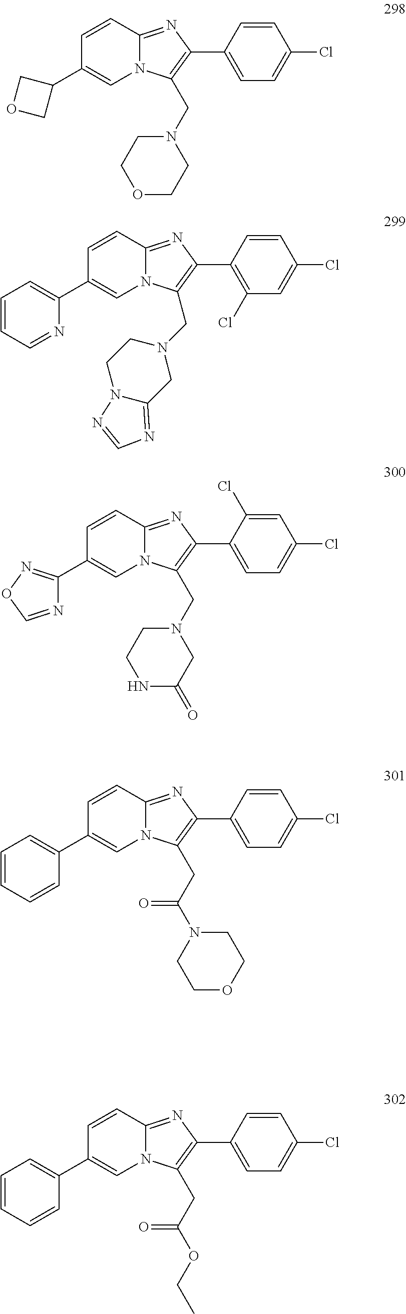

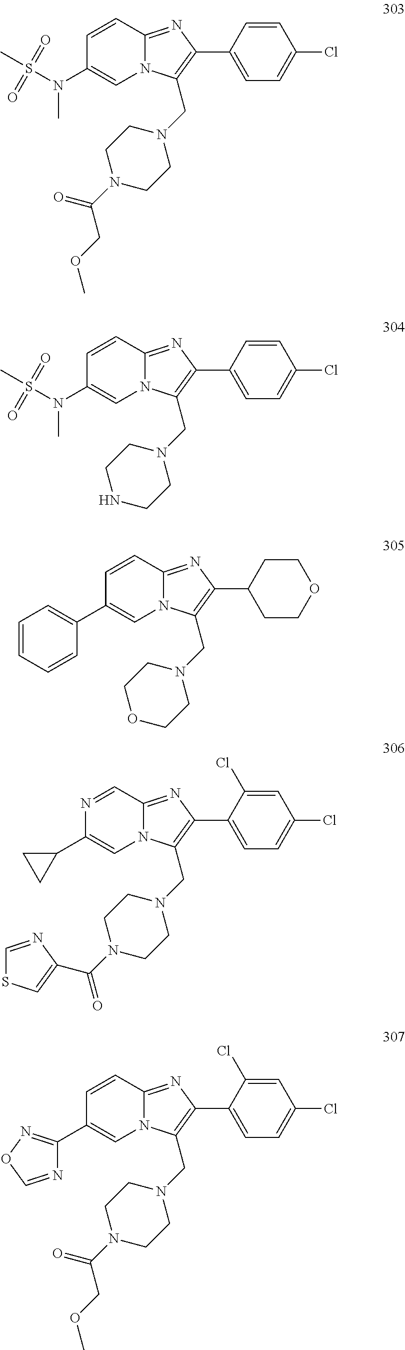 [SMILES: COCC(=O)N1CCN(Cc2c(-c3ccc(Cl)cc3)nc3ccc(N(C)S(C)(=O)=O)cn23)CC1 ; CN(c1ccc2nc(-c3ccc(Cl)cc3)c(CN3CCNCC3)n2c1)S(C)(=O)=O ; c1ccc(-c2ccc3nc(C4CCOCC4)c(CN4CCOCC4)n3c2)cc1 ; O=C(c1cscn1)N1CCN(Cc2c(-c3ccc(Cl)cc3Cl)nc3cnc(C4CC4)cn23)CC1 ; COCC(=O)N1CCN(Cc2c(-c3ccc(Cl)cc3Cl)nc3ccc(-c4ncon4)cn23)CC1]

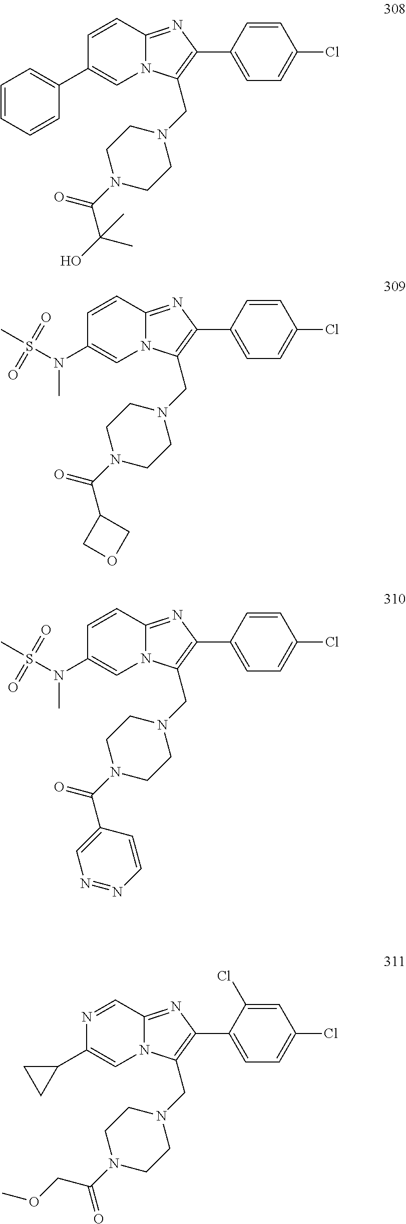 [SMILES: CC(C)(O)C(=O)N1CCN(Cc2c(-c3ccc(Cl)cc3)nc3ccc(-c4ccccc4)cn23)CC1 ; CN(c1ccc2nc(-c3ccc(Cl)cc3)c(CN3CCN(C(=O)C4COC4)CC3)n2c1)S(C)(=O)=O ; CN(c1ccc2nc(-c3ccc(Cl)cc3)c(CN3CCN(C(=O)c4ccnnc4)CC3)n2c1)S(C)(=O)=O ; COCC(=O)N1CCN(Cc2c(-c3ccc(Cl)cc3Cl)nc3cnc(C4CC4)cn23)CC1]

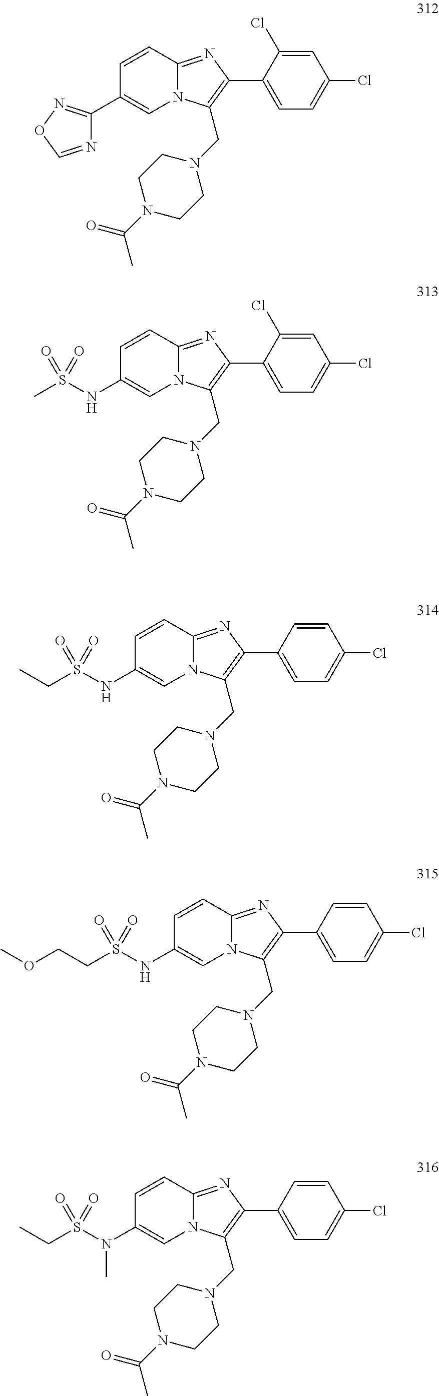 [SMILES: CC(=O)N1CCN(Cc2c(-c3ccc(Cl)cc3Cl)nc3ccc(-c4ncon4)cn23)CC1 ; CC(=O)N1CCN(Cc2c(-c3ccc(Cl)cc3Cl)nc3ccc(NS(C)(=O)=O)cn23)CC1 ; CCS(=O)(=O)Nc1ccc2nc(-c3ccc(Cl)cc3)c(CN3CCN(C(C)=O)CC3)n2c1 ; COCCS(=O)(=O)Nc1ccc2nc(-c3ccc(Cl)cc3)c(CN3CCN(C(C)=O)CC3)n2c1 ; CCS(=O)(=O)N(C)c1ccc2nc(-c3ccc(Cl)cc3)c(CN3CCN(C(C)=O)CC3)n2c1]

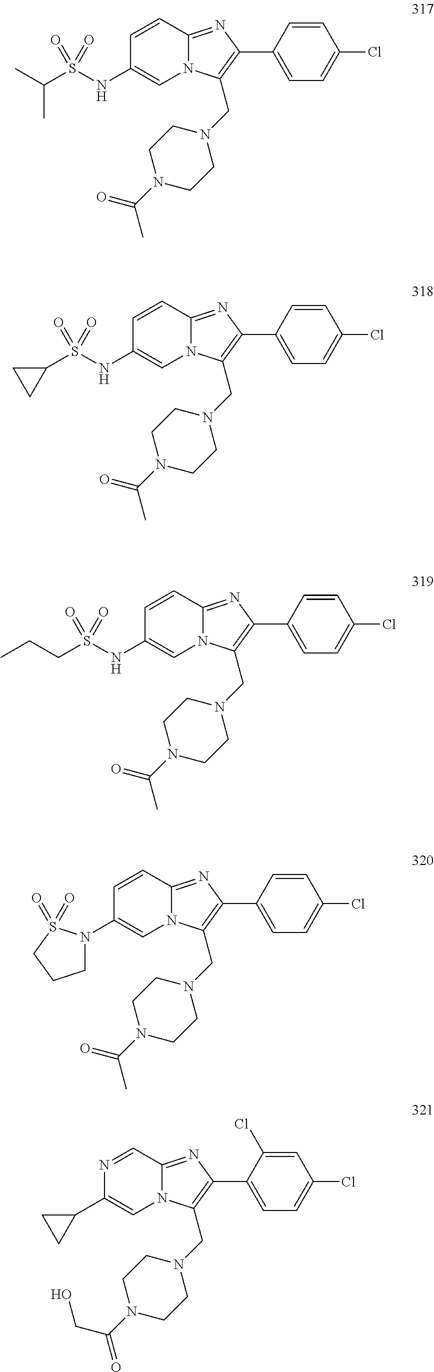 [SMILES: CC(=O)N1CCN(Cc2c(-c3ccc(Cl)cc3)nc3ccc(NS(=O)(=O)C(C)C)cn23)CC1 ; CC(=O)N1CCN(Cc2c(-c3ccc(Cl)cc3)nc3ccc(NS(=O)(=O)C4CC4)cn23)CC1 ; CCCS(=O)(=O)Nc1ccc2nc(-c3ccc(Cl)cc3)c(CN3CCN(C(C)=O)CC3)n2c1 ; CC(=O)N1CCN(Cc2c(-c3ccc(Cl)cc3)nc3ccc(N4CCCS4(=O)=O)cn23)CC1 ; O=C(CO)N1CCN(Cc2c(-c3ccc(Cl)cc3Cl)nc3cnc(C4CC4)cn23)CC1]

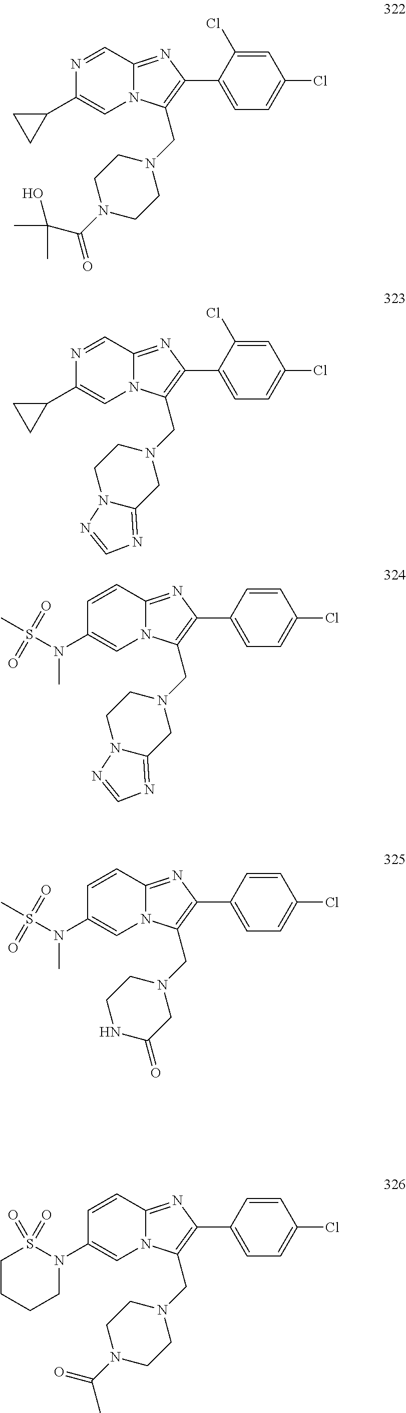

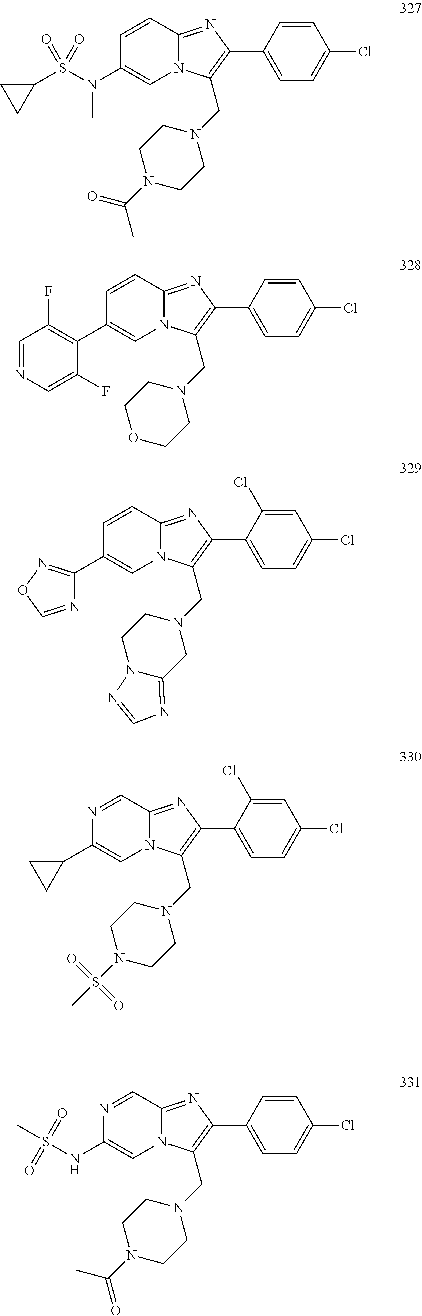

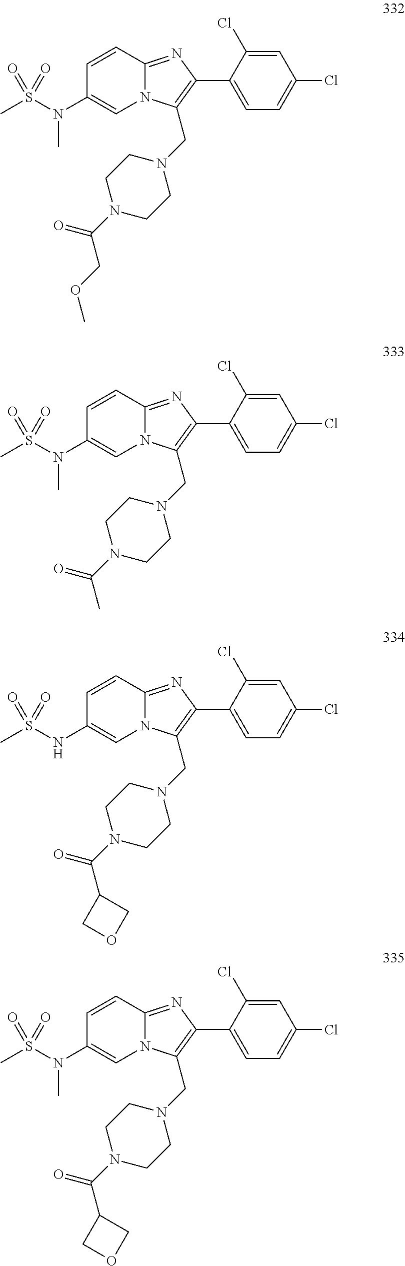 [SMILES: COCC(=O)N1CCN(Cc2c(-c3ccc(Cl)cc3Cl)nc3ccc(N(C)S(C)(=O)=O)cn23)CC1 ; CC(=O)N1CCN(Cc2c(-c3ccc(Cl)cc3Cl)nc3ccc(N(C)S(C)(=O)=O)cn23)CC1 ; CS(=O)(=O)Nc1ccc2nc(-c3ccc(Cl)cc3Cl)c(CN3CCN(C(=O)C4COC4)CC3)n2c1 ; CN(c1ccc2nc(-c3ccc(Cl)cc3Cl)c(CN3CCN(C(=O)C4COC4)CC3)n2c1)S(C)(=O)=O]

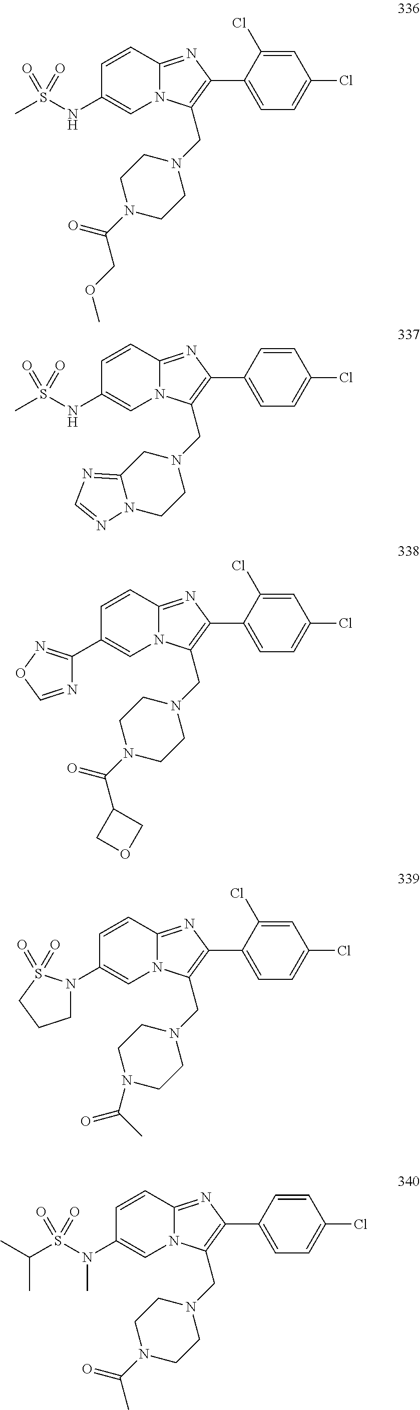

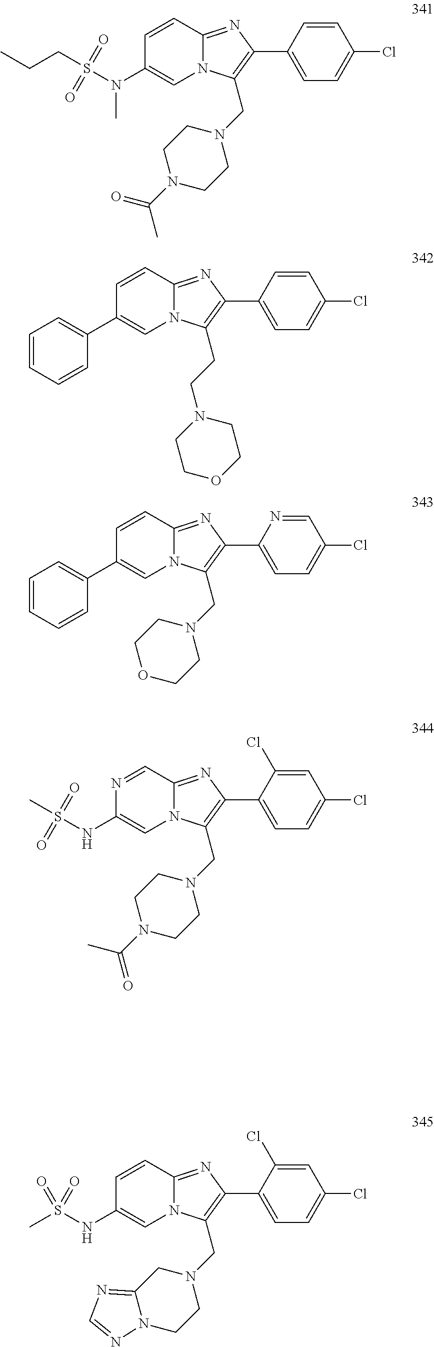

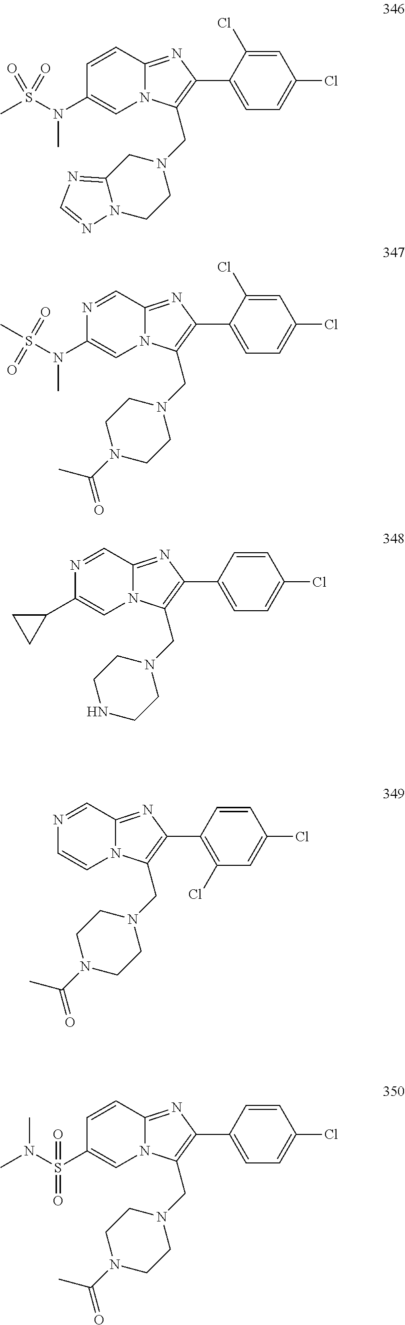

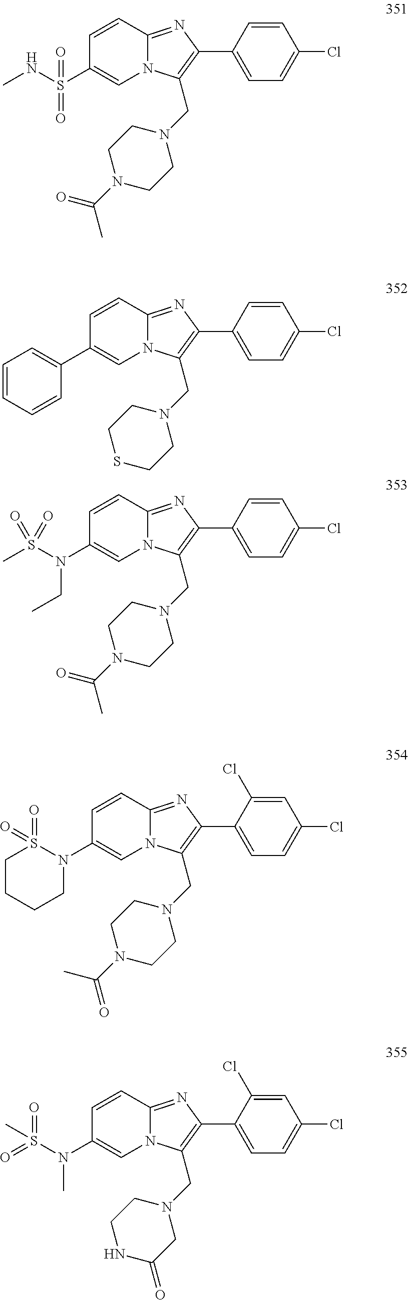

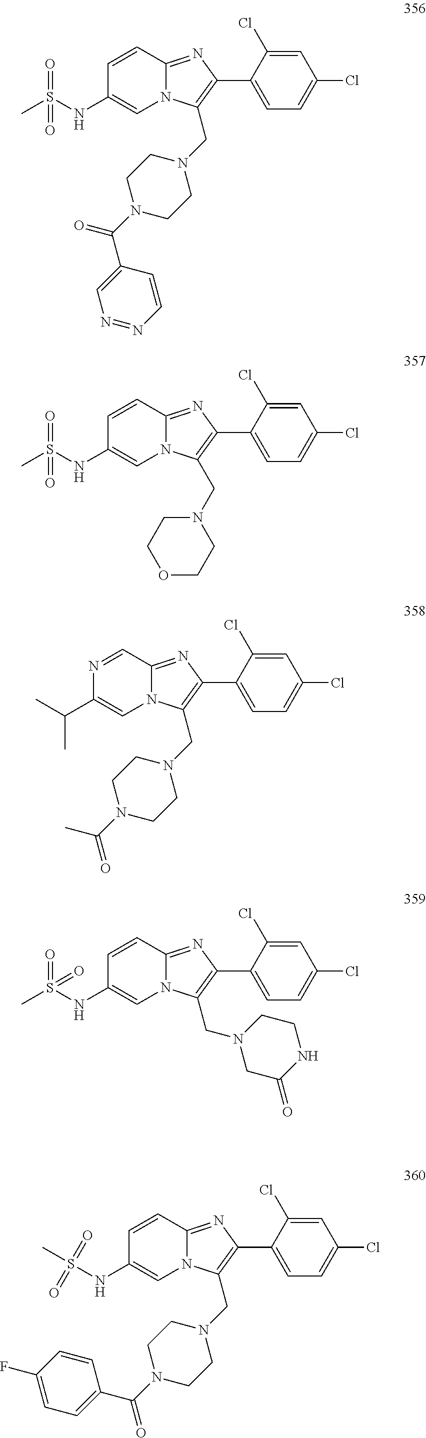 [SMILES: CS(=O)(=O)Nc1ccc2nc(-c3ccc(Cl)cc3Cl)c(CN3CCN(C(=O)c4ccnnc4)CC3)n2c1 ; CS(=O)(=O)Nc1ccc2nc(-c3ccc(Cl)cc3Cl)c(CN3CCOCC3)n2c1 ; CC(=O)N1CCN(Cc2c(-c3ccc(Cl)cc3Cl)nc3cnc(C(C)C)cn23)CC1 ; CS(=O)(=O)Nc1ccc2nc(-c3ccc(Cl)cc3Cl)c(CN3CCNC(=O)C3)n2c1 ; CS(=O)(=O)Nc1ccc2nc(-c3ccc(Cl)cc3Cl)c(CN3CCN(C(=O)c4ccc(F)cc4)CC3)n2c1]

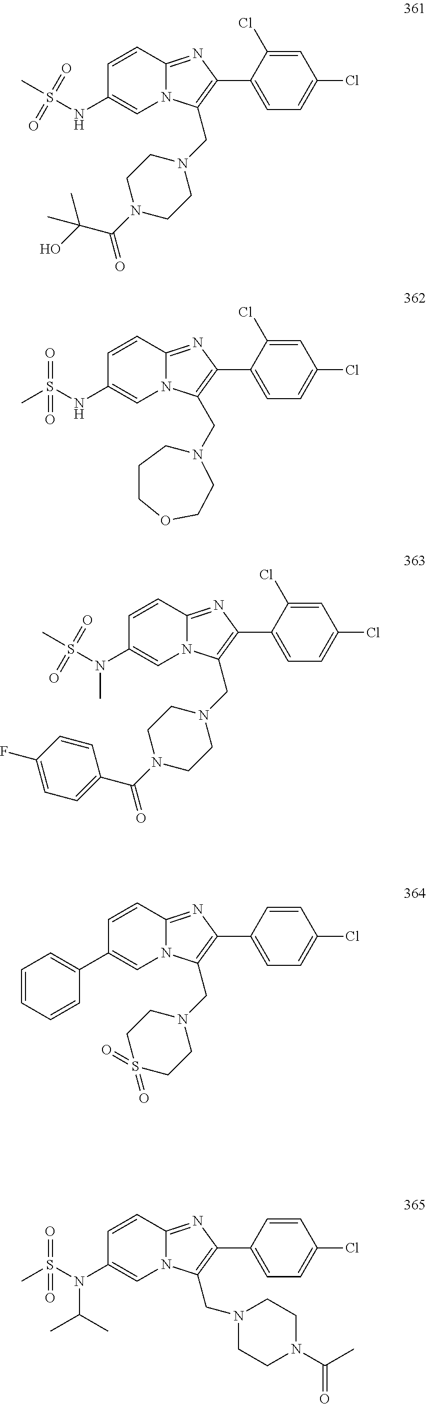 [SMILES: CC(C)(O)C(=O)N1CCN(Cc2c(-c3ccc(Cl)cc3Cl)nc3ccc(NS(C)(=O)=O)cn23)CC1 ; CS(=O)(=O)Nc1ccc2nc(-c3ccc(Cl)cc3Cl)c(CN3CCCOCC3)n2c1 ; CN(c1ccc2nc(-c3ccc(Cl)cc3Cl)c(CN3CCN(C(=O)c4ccc(F)cc4)CC3)n2c1)S(C)(=O)=O ; O=S1(=O)CCN(Cc2c(-c3ccc(Cl)cc3)nc3ccc(-c4ccccc4)cn23)CC1 ; CC(=O)N1CCN(Cc2c(-c3ccc(Cl)cc3)nc3ccc(N(C(C)C)S(C)(=O)=O)cn23)CC1]

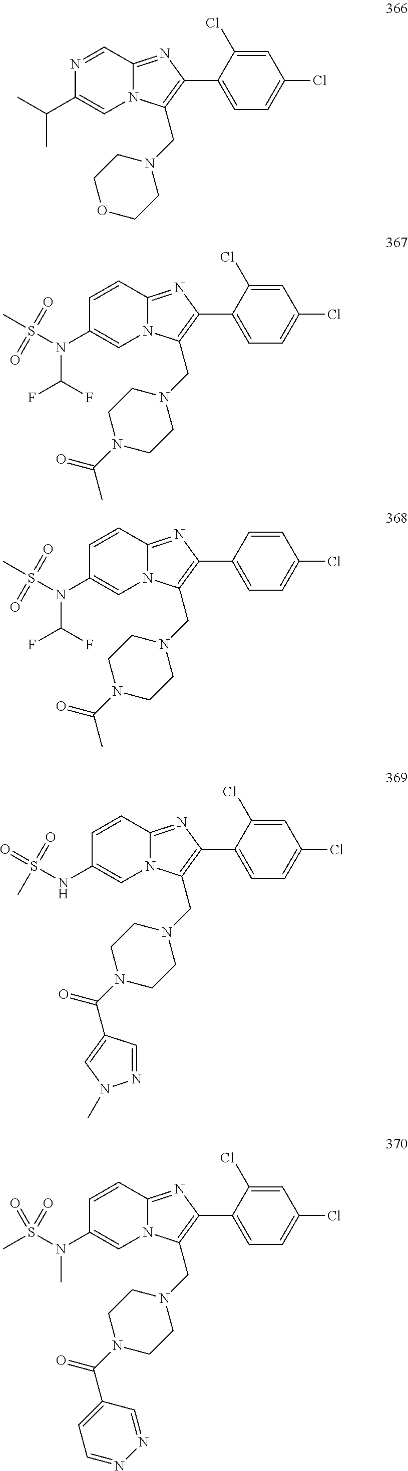

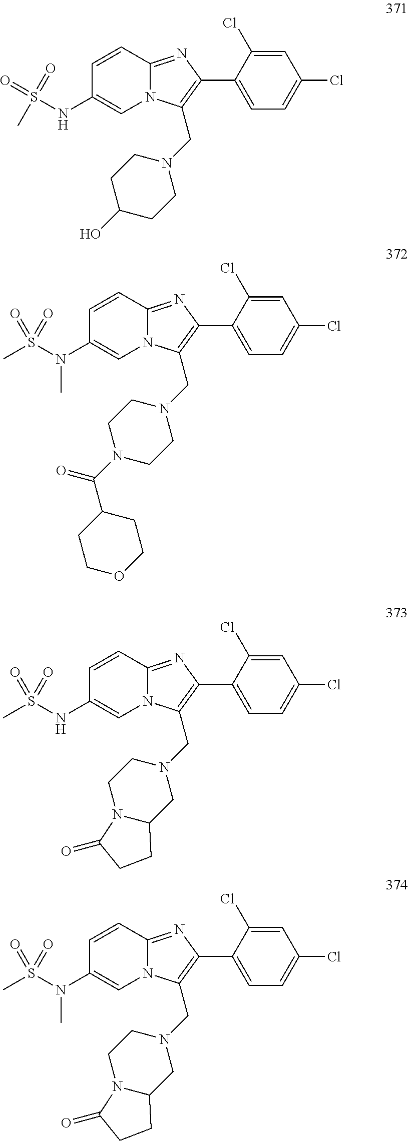 [SMILES: CS(=O)(=O)Nc1ccc2nc(-c3ccc(Cl)cc3Cl)c(CN3CCC(O)CC3)n2c1 ; CN(c1ccc2nc(-c3ccc(Cl)cc3Cl)c(CN3CCN(C(=O)C4CCOCC4)CC3)n2c1)S(C)(=O)=O ; CS(=O)(=O)Nc1ccc2nc(-c3ccc(Cl)cc3Cl)c(CN3CCN4C(=O)CCC4C3)n2c1 ; CN(c1ccc2nc(-c3ccc(Cl)cc3Cl)c(CN3CCN4C(=O)CCC4C3)n2c1)S(C)(=O)=O]

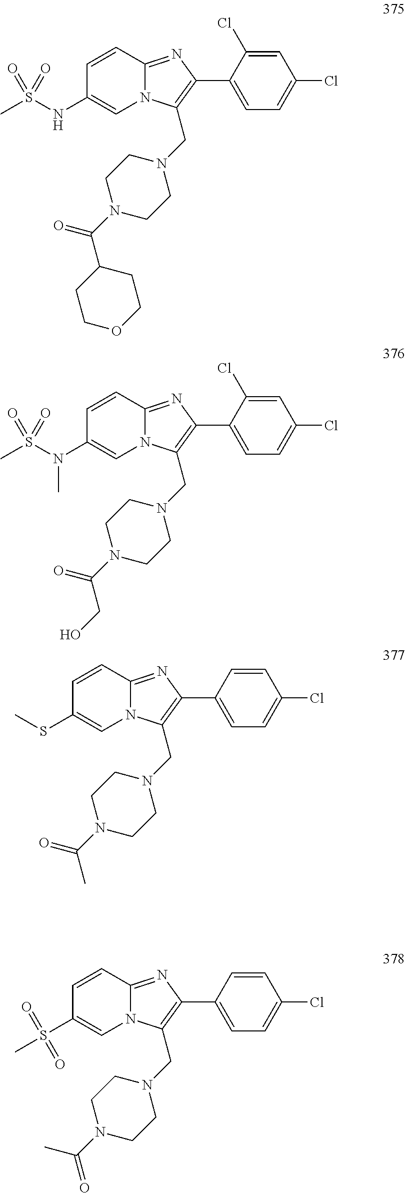

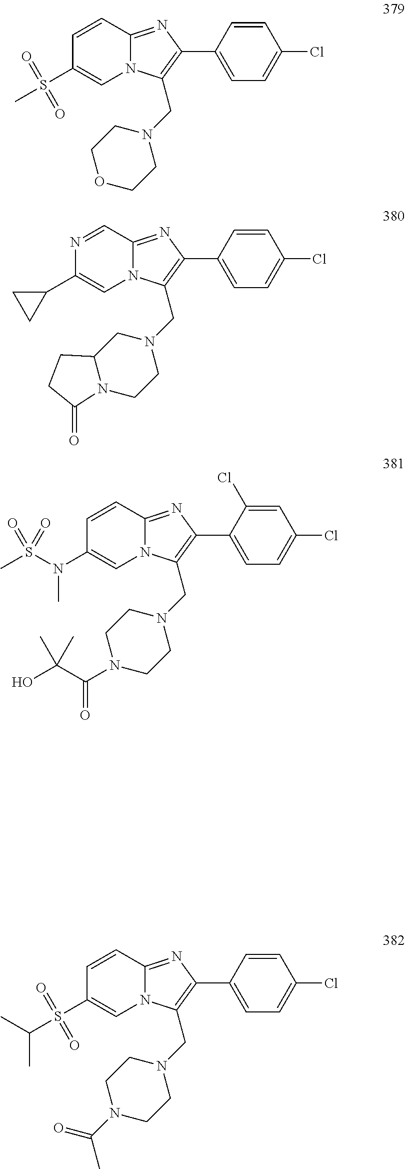

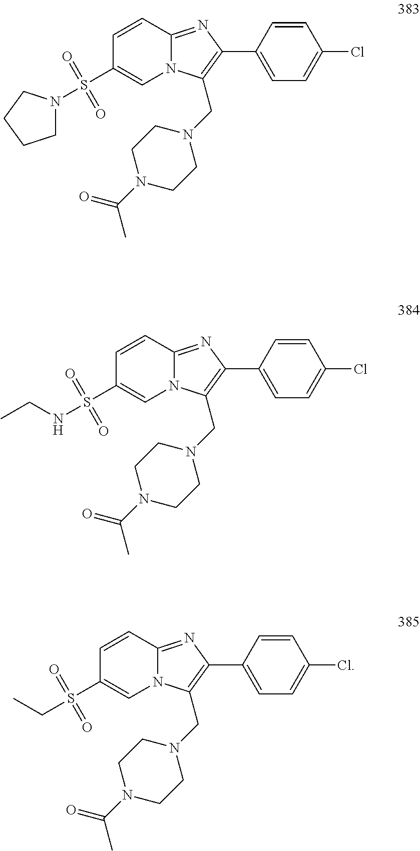 [SMILES: CC(=O)N1CCN(Cc2c(-c3ccc(Cl)cc3)nc3ccc(S(=O)(=O)N4CCCC4)cn23)CC1 ; CCNS(=O)(=O)c1ccc2nc(-c3ccc(Cl)cc3)c(CN3CCN(C(C)=O)CC3)n2c1 ; CCS(=O)(=O)c1ccc2nc(-c3ccc(Cl)cc3)c(CN3CCN(C(C)=O)CC3)n2c1]

43. A method according to claim 42, wherein HNR⁷⁷R⁷⁸ is selected from a substituted or unsubstituted piperazine compound, and a substituted or unsubstituted morpholine compound.

44. A method for screening for a SLC2A class I transporter inhibitor compound having a structure as defined in claim 34, which method comprises
(a) contacting a receptor having a SLC2A class I transporter function with a test compound;
(b) measuring the transport of a species across the receptor, which species is one whose transport is facilitated by a SLC2A class I transporter; and
(c) determining whether the test compound is a SLC2A class I transporter inhibitor from the measurements taken in step (b).

45. The method according to claim 44, which method comprises:
(a) contacting a cell comprising a SLC2A class I transporter with a test compound;
(b) measuring the transport of a species across a membrane of the cell, which species is one whose transport across the membrane is facilitated by a SLC2A class I transporter; and
(c) determining whether the test compound is a SLC2A class I transporter inhibitor from the measurements taken in step (b).

46. The method according to claim 44, wherein the species is selected from a substituted or unsubstituted carbohydrate compound, a substituted or unsubstituted sugar compound, and a mixture of two or more of the above.

47. The method according to claim 46, wherein the species comprises a substituted or unsubstituted glucose, preferably radiolabelled glucose.

48. The method according to any of claim 45, wherein the cell is a cell that has been transfected such that it comprises a SLC2A class I transporter at its surface.

49. The method according to claim 34, wherein the SLC2A class I transporter is selected from GLUT1, GLUT2, GLUT3, GLUT4 and GLUT14.

50. A compound for use in medicine according to claim 8, wherein R⁷³ comprises a substituted or unsubstituted group comprising a carbonyl group; and/or wherein R¹ is H, R² is H, R⁴ is H or Me, R³ is not H, R⁶ is not H, each R⁷¹ is H, and each R⁷² is H.

51. A compound for use in medicine according to claim 8 or claim 50, wherein R⁷³ comprises an unsubstituted or substituted acyl group; and/or wherein X is N; and/or wherein R¹ is H, R² is H, R⁴ is H or Me, R³ is not H, R⁶ is not H, each R⁷¹ is H, and each R⁷² is H.
